(19) United States
(12) Patent Application Publication
(10) Pub. No.: US 2024/0251666 A1
(43) Pub. Date: Jul. 25, 2024

(54) HETEROCYCLIC COMPOUND, ORGANIC LIGHT EMITTING DEVICE AND COMPOSITION FOR ORGANIC MATERIAL LAYER COMPRISING THE SAME

(71) Applicant: LT MATERIALS CO., LTD., Yongin-si (KR)

(72) Inventors: Sol LEE, Yongin-si (KR); Jun Tae Mo, Yongin-si (KR); Dong Jun Kim, Yongin-si (KR); Dae Hyuk Choi, Yongin-si (KR)

(73) Assignee: LT MATERIALS CO., LTD., Yongin-si (KR)

(21) Appl. No.: 18/514,459

(22) Filed: Nov. 20, 2023

(30) Foreign Application Priority Data
Nov. 29, 2022 (KR) 10-2022-0162609

(51) Int. Cl. H10K 85/60; C07D 241/38; C07D 403/04; C07D 403/14; C07D 405/04; C07D 405/12; C07D 405/14; C07D 409/04; C07D 409/12; C07D 491/048; C07D 491/153; C07D 495/04; C07D 495/14

(52) U.S. Cl. CPC H10K 85/636; C07D 241/38; C07D 403/04; C07D 403/14; C07D 405/04; C07D 405/12; C07D 405/14; C07D 409/04; C07D 409/12; C07D 491/048; C07D 491/153; C07D 495/04; C07D 495/14; H10K 85/626; H10K 85/633; H10K 85/654; H10K 85/657; H10K 85/6572; H10K 85/6574; H10K 50/11

(57) ABSTRACT

The present specification relates to a heterocyclic compound represented by the following Chemical Formula 1, and an organic light emitting device and a composition for an organic material layer including the same:

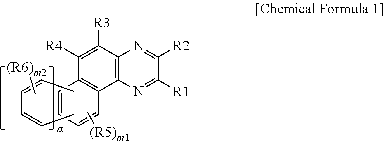

in Chemical Formula 1,
a is an integer of 0 or 1,
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; substituted or unsubstituted C2 to C60 heterocycloalkyl group; a substituted or unsubstituted C6 to C60 aryl group; a substituted or unsubstituted C2 to C60 heteroaryl group; a group represented by the following Chemical Formula 2-1; and a group represented by the following Chemical Formula 2-2,
at least one of R5 and R6 is the group represented by the following Chemical Formula 2-1; or the group represented by the following Chemical Formula 2-2, and
m1 and m2 are each independently one of integers of 1 to 4, and when a is 1, m1 is an integer of 1 or 2,

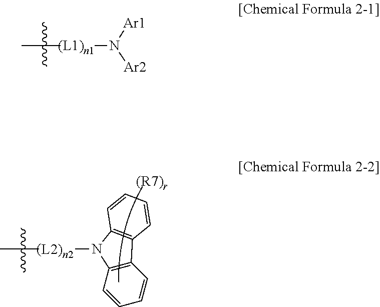

in Chemical Formula 2-1 or Chemical Formula 2-2,
Ar1 and Ar2 are the same as or different from each other, and each independently a substituted or unsubstituted C6 to C60 aryl group; or a substituted or unsubstituted C2 to C60 heteroaryl group,
R7s are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heteroring,
r is one of integers of 1 to 8,
L1 and L2 are the same as or different from each other, and each independently a single bond; a substituted or unsubstituted C6 to C60 arylene group; or a substituted or unsubstituted C2 to C60 heteroarylene group, and
n1 and n2 are the same as or different from each other, and each independently one of integers of 0 to 3, and when n1 and n2 are 2 or greater, L1s or L2s are the same as or different from each other.

### CROSS-REFERENCE TO RELATED APPLICATION (S)

This application claims priority to Korean Patent Application No. 10-2022-0162609, filed on Nov. 29, 2022, the disclosure of which is incorporated herein by reference in its entirety.

### TECHNICAL FIELD

The present disclosure relates to a heterocyclic compound, and an organic light emitting device and a composition for an organic material layer including the same.

### DESCRIPTION OF THE RELATED ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light is emitted as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of serving as a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### SUMMARY

The present disclosure is directed to providing a heterocyclic compound, and an organic light emitting device and a composition for an organic material layer including the same. One embodiment of the present disclosure provides a heterocyclic compound represented by the following Chemical Formula 1:

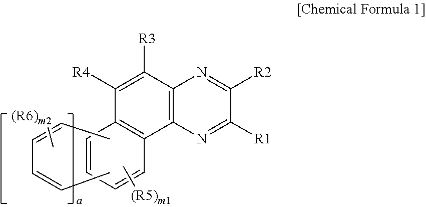

in Chemical Formula 1,
a is an integer of 0 or 1,
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a group represented by the following Chemical Formula 2-1; and a group represented by the following Chemical Formula 2-2,
at least one of R5 and R6 is the group represented by the following Chemical Formula 2-1; or the group represented by the following Chemical Formula 2-2, and
m1 and m2 are each independently one of integers of 1 to 4, and when a is 1, m1 is an integer of 1 or 2,

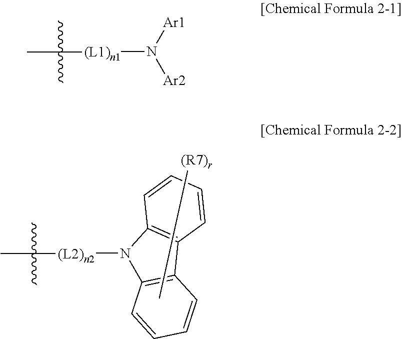

in Chemical Formula 2-1 or Chemical Formula 2-2,
Ar1 and Ar2 are the same as or different from each other, and each independently a substituted or unsubstituted C6 to C60 aryl group; or a substituted or unsubstituted C2 to C60 heteroaryl group,
R7s are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heteroring,
r is one of integers of 1 to 8,
L1 and L2 are the same as or different from each other, and each independently a single bond; a substituted or unsubstituted C6 to C60 arylene group; or a substituted or unsubstituted C2 to C60 heteroarylene group, and
n1 and n2 are the same as or different from each other, and each independently one of integers of 0 to 3, and when n1 and n2 are 2 or greater, L1s or L2s are the same as or different from each other.

In addition, one embodiment of the present disclosure provides an organic light emitting device including: a first electrode; a second electrode provided opposite to the first electrode; and one or more organic material layers provided between the first electrode and the second electrode, wherein one or more layers of the organic material layers include the heterocyclic compound according to the present disclosure.

In addition, one embodiment of the present disclosure provides a composition for an organic material layer of an organic light emitting device, the composition including: the heterocyclic compound represented by Chemical Formula 1 according to the present disclosure; and a heterocyclic compound represented by Chemical Formula A.

A heterocyclic compound according to one embodiment can be used as an organic material layer material of an organic light emitting device. The compound is capable of performing roles of a hole injection layer material, an electron blocking layer material, a hole transport layer material, a light emitting layer material, an electron transport layer material, a hole blocking layer material, an electron injection layer material and the like in an organic light emitting device. Particularly, the compound can be used as a light emitting layer material of an organic light emitting device.

Specifically, the heterocyclic compound can be used as a light emitting material either alone or as a mixture with a P-type host, and may be used as a host material or a dopant material of a light emitting layer.

In addition, when using the compound represented by Chemical Formula 1 in an organic material layer of an organic light emitting device, it is possible to lower a driving voltage of the organic light emitting device, and enhance light emission efficiency and lifetime properties of the organic light emitting device due to an increase in hole mobility.

### BRIEF DESCRIPTION OF THE DRAWINGS

FIGS. 1 to 4 are diagrams each schematically illustrating a lamination structure of an organic light emitting device according to one embodiment of the present disclosure.

### DESCRIPTION OF SPECIFIC EMBODIMENTS

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a trifluoromethyl group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; -P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted.

In the present specification, R, R′ and R″ are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In the present specification, “the number of protons” means the number of substituents that a specific compound may have, and specifically, the number of protons may mean the number of hydrogens. For example, unsubstituted benzene may be expressed to have the number of protons of 5, an unsubstituted naphthyl group may be expressed to have the number of protons of 7, a naphthyl group substituted with a phenyl group may be expressed to have the number of protons of 6, and an unsubstituted biphenyl group may be expressed to have the number of protons of 9.

In the present specification, the halogen may be fluorine, chlorine, bromine or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples of the alkyl group may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl a group, a 1-methylhexyl group, cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto. In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples of the alkenyl group may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples of the alkoxy group may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy an group, group, n-hexyloxy a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples of the cycloalkyl group may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by -P(═O) R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

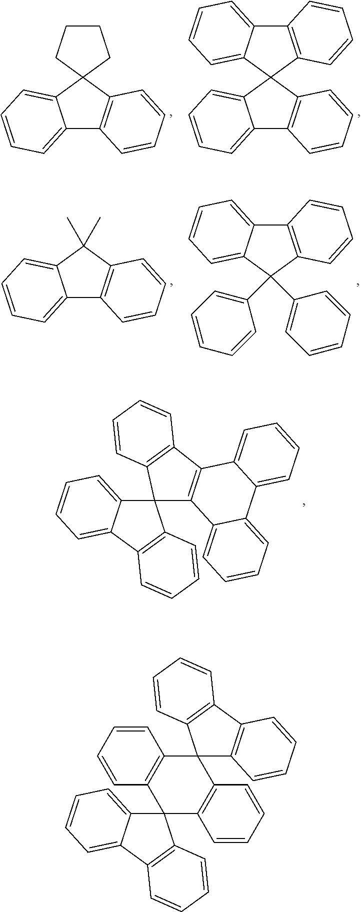

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

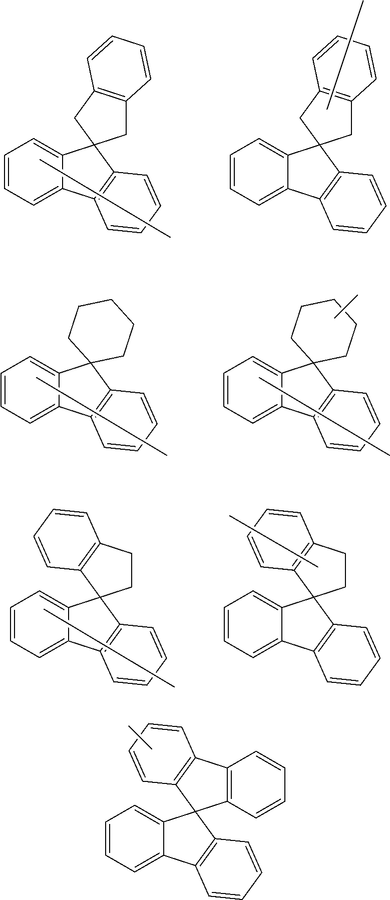

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilole group, a spirobi (dibenzosilole) group, a dihydrophenazinyl group, a phenoxazinyl group, a phenanthridyl group, a thienyl group, an indolo[2,3-a]carbazolyl group, an indolo[2,3-b] carbazolyl group, an indolinyl group, a 10,11-dihydro-dibenzo[b, f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b, e][1, 4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

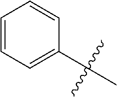 [SMILES: *c1ccccc1]

may mean that the total number of substituents that the phenyl group may have is 5 (T1 in the formula), and the number of deuterium atoms among these is 1 (T2 in the formula). In other words, having a deuterium content of 20% in a phenyl group may be represented by the following structural formulae.

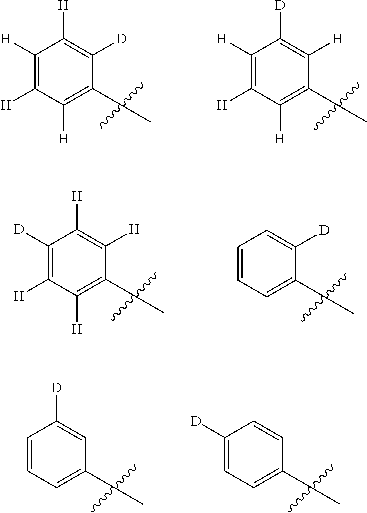 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include benzene, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present disclosure provides a heterocyclic compound represented by the following Chemical Formula 1:

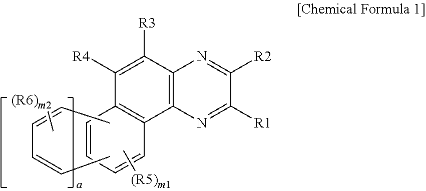

in Chemical Formula 1,
a is an integer of 0 or 1,
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a group represented by the following Chemical Formula 2-1; and a group represented by the following Chemical Formula 2-2,
at least one of R5 and R6 is the group represented by the following Chemical Formula 2-1; or the group represented by the following Chemical Formula 2-2, and
m1 and m2 are each independently one of integers of 1 to 4, and when a is 1, m1 is an integer of 1 or 2,

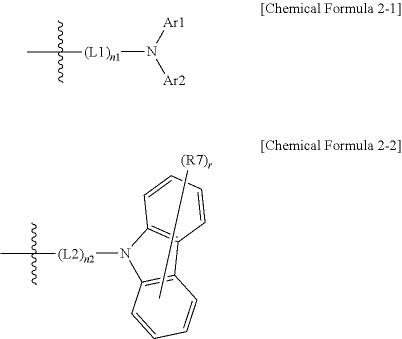

in Chemical Formula 2-1 or Chemical Formula 2-2,
Ar1 and Ar2 are the same as or different from each other, and each independently a substituted or unsubstituted C6 to C60 aryl group; or a substituted or unsubstituted C2 to C60 heteroaryl group,
R7s are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heteroring,
r is one of integers of 1 to 8,
L1 and L2 are the same as or different from each other, and each independently a single bond; a substituted or unsubstituted C6 to C60 arylene group; or a substituted or unsubstituted C2 to C60 heteroarylene group, and
n1 and n2 are the same as or different from each other, and each independently one of integers of 0 to 3, and when n1 and n2 are 2 or greater, L1s or L2s are the same as or different from each other.

In one embodiment of the present disclosure, the heterocyclic compound represented by Chemical Formula 1 may be represented by the following Chemical Formula 1-a or Chemical Formula 1-b:

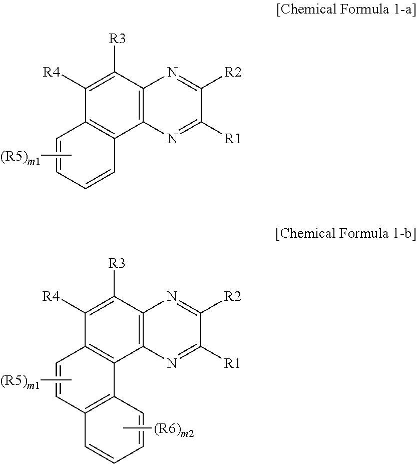

in Chemical Formula 1-a and Chemical Formula 1-b,
R1 to R6, m1 and m2 have the same definitions as in Chemical Formula 1.

In one embodiment of the present disclosure, the heterocyclic compound represented by Chemical Formula 1 may be represented by the following Chemical Formula 1-a-1 or Chemical Formula 1-b-1:

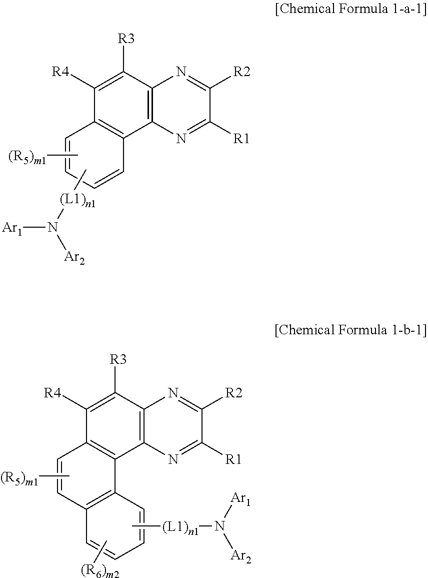

in Chemical Formula 1-a-1 and Chemical Formula 1-b-1,
R1 to R6, L1, Ar1, Ar2, n1, m1 and m2 have the same definitions as in Chemical Formula 1.

In one embodiment of the present disclosure, the heterocyclic compound represented by Chemical Formula 1 may be represented by any one of the following Chemical Formula 1-a-2 and Chemical Formula 1-b-2:

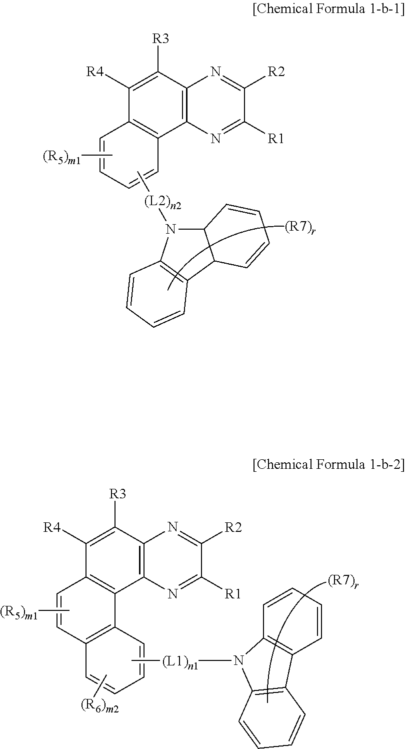

in Chemical Formula 1-a-2 and Chemical Formula 1-b-2,
R1 to R7, L2, n2, m1, m2 and r have the same definitions as in Chemical Formula 1.

In one embodiment of the present disclosure, the heterocyclic compound represented by Chemical Formula 1 may be represented by any one of the following Chemical Formula 1-a-1-1, Chemical Formula 1-a-1-2, Chemical Formula 1-a-2-1, Chemical Formula 1-a-2-2, Chemical Formula 1-b-1-1, Chemical Formula 1-b-1-2, Chemical Formula 1-b-2-1 and Chemical Formula 1-b-2-2:

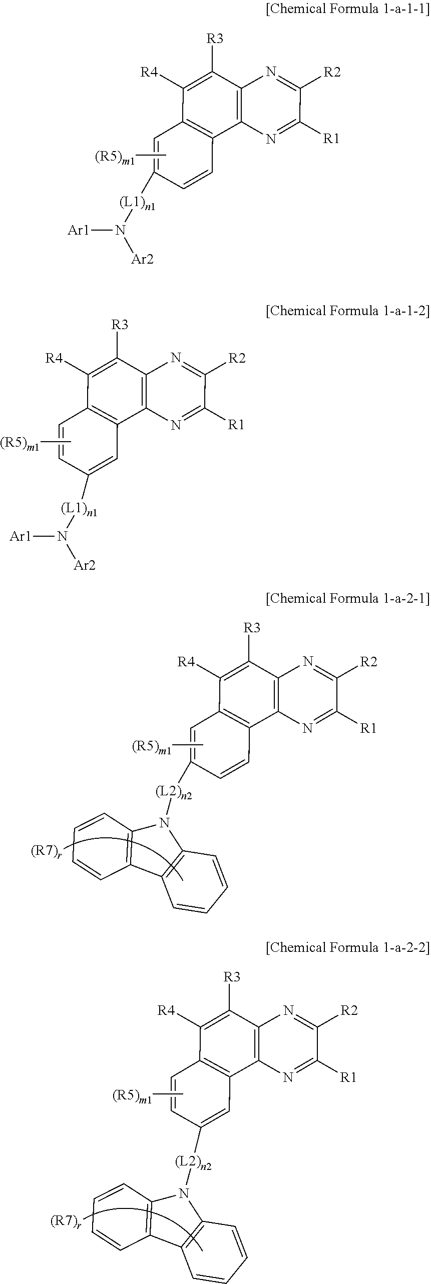

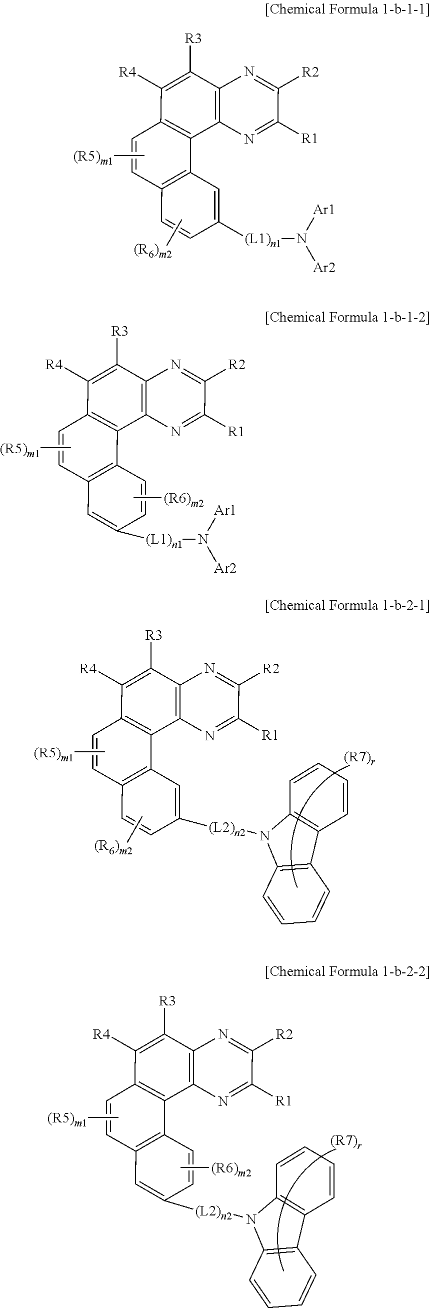

in Chemical Formula 1-a-1-1, Chemical Formula 1-a-1-2, Chemical Formula 1-a-2-1, Chemical Formula 1-a-2-2, Chemical Formula 1-b-1-1, Chemical Formula 1-b-1-2, Chemical Formula 1-b-2-1 and Chemical Formula 1-b-2-2,
R1 to R7, L1, L2, Ar1, Ar2, n1, n2, m1, m2 and r have the same definitions as in Chemical Formula 1.

In one embodiment of the present disclosure, R1 to R6 are the same as or different from each other, and may be each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; the group represented by Chemical Formula 2-1; and the group represented by Chemical Formula 2-2.

In one embodiment of the present disclosure, R1 to R6 are the same as or different from each other, and may be each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; substituted or unsubstituted C2 to C20 heterocycloalkyl group; a substituted or unsubstituted C6 to C20 aryl group; a substituted or unsubstituted C2 to C20 heteroaryl group; the group represented by Chemical Formula 2-1; and the group represented by Chemical Formula 2-2.

In one embodiment of the present disclosure, R1 to R6 are the same as or different from each other, and may be each independently selected from the group consisting of hydrogen; deuterium; halogen; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; a substituted or unsubstituted C2 to C10 heteroaryl group; the group represented by Chemical Formula 2-1; and the group represented by Chemical Formula 2-2.

In one embodiment of the present disclosure, R1 to R6 are the same as or different from each other, and may be each independently selected from the group consisting of hydrogen; deuterium; halogen; a cyano group; a substituted or unsubstituted C6 to C60 aryl group; a substituted or unsubstituted C2 to C60 heteroaryl group; the group represented by Chemical Formula 2-1; and the group represented by Chemical Formula 2-2.

In one embodiment of the present disclosure, R1 to R6 are the same as or different from each other, and may be each independently selected from the group consisting of hydrogen; deuterium; halogen; a cyano group; a substituted or unsubstituted C6 to C30 aryl group; a substituted or unsubstituted C2 to C30 heteroaryl group; the group represented by Chemical Formula 2-1; and the group represented by Chemical Formula 2-2.

In one embodiment of the present disclosure, R1 to R6 are the same as or different from each other, and may be each independently selected from the group consisting of hydrogen; deuterium; halogen; a cyano group; a substituted or unsubstituted C6 to C10 aryl group; a substituted or unsubstituted C2 to C10 heteroaryl group; the group represented by Chemical Formula 2-1; and the group represented by Chemical Formula 2-2.

In one embodiment of the present disclosure, R1 to R6 are the same as or different from each other, and may be each independently selected from the group consisting of hydrogen; deuterium; a substituted or unsubstituted C6 to C10 aryl group; the group represented by Chemical Formula 2-1; and the group represented by Chemical Formula 2-2.

In one embodiment of the present disclosure, R1 and R2 are the same as or different from each other, and may be each independently a substituted or unsubstituted C6 to C60 aryl group.

In one embodiment of the present disclosure, when a is 0, R5 may be the group represented by Chemical Formula 2-1 or the group represented by Chemical Formula 2-2.

In one embodiment of the present disclosure, when a is 1, R5 may be the group represented by Chemical Formula 2-1 or the group represented by Chemical Formula 2-2.

In one embodiment of the present disclosure, when a is 1, R6 may be the group represented by Chemical Formula 2-1 or the group represented by Chemical Formula 2-2.

In one embodiment of the present disclosure, m1 and m2 may be each independently one of integers of 1 to 4.

In one embodiment of the present disclosure, when a is 0, m1 may be one of integers of 1 to 4.

In one embodiment of the present disclosure, when a is 1, m1 may be one of integers of 1 and 2, and m2 may be one of integers of 1 to 4.

In one embodiment of the present disclosure, Ar1 and Ar2 are the same as or different from each other, and may be each independently a substituted or unsubstituted C6 to C40 aryl group or a substituted or unsubstituted C2 to C40 heteroaryl group.

In one embodiment of the present disclosure, Ar1 and Ar2 are the same as or different from each other, and may be each independently a substituted or unsubstituted C6 to C20 aryl group or a substituted or unsubstituted C2 to C20 heteroaryl group.

In one embodiment of the present disclosure, Ar1 and Ar2 are the same as or different from each other and may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted dibenzothiophenyl group; or a substituted or unsubstituted dibenzofuranyl group, but are not limited to these examples.

In the “substituted” phenyl group, the “substituted” biphenyl group, the “substituted” terphenyl group, the “substituted” naphthyl group, the “substituted” fluorenyl group, the “substituted” dibenzothiophenyl group and the “substituted” dibenzofuranyl group, the “substituted” may refer to being substituted with a halogen group; a cyano group; a methyl group; a trifluoromethyl group; or a tert-butyl group. For example, the “substituted” fluorenyl group may include a 9,9-dimethylfluorenyl group. However, the substituted groups are not limited to these examples.

In one embodiment of the present disclosure, R7s are the same as or different from each other, and may be each independently selected from the group consisting of hydrogen; deuterium; halogen; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C6 to C40 aryl group; and a substituted or unsubstituted C2 to C40 heteroaryl group.

In one embodiment of the present disclosure, R7s are the same as or different from each other, and may be each independently selected from the group consisting of hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C6 to C20 aryl group; and a substituted or unsubstituted C2 to C20 heteroaryl group.

In one embodiment of the present disclosure, when r is 2 or greater, two or more groups of R7s adjacent to each other may bond to each other to form a substituted or unsubstituted C6 to C60 aromatic hydrocarbon ring; or a substituted or unsubstituted C2 to C60 heteroring.

In one embodiment of the present disclosure, when r is 2 or greater, two or more groups of R7s adjacent to each other may bond to each other to form a substituted or unsubstituted C6 to C40 aromatic hydrocarbon ring; or a substituted or unsubstituted C2 to C40 heteroring.

In one embodiment of the present disclosure, when r is 2 or greater, two or more groups of R7s adjacent to each other may bond to each other to form a substituted or unsubstituted C6 to C20 aromatic hydrocarbon ring; or a substituted or unsubstituted C2 to C20 heteroring.

In one embodiment of the present disclosure, the group represented by Chemical Formula 2-2 may be any one of groups represented by the following Chemical Formula 2-2-a, Chemical Formula 2-2-b, Chemical Formula 2-2-c and Chemical Formula 2-2-d:

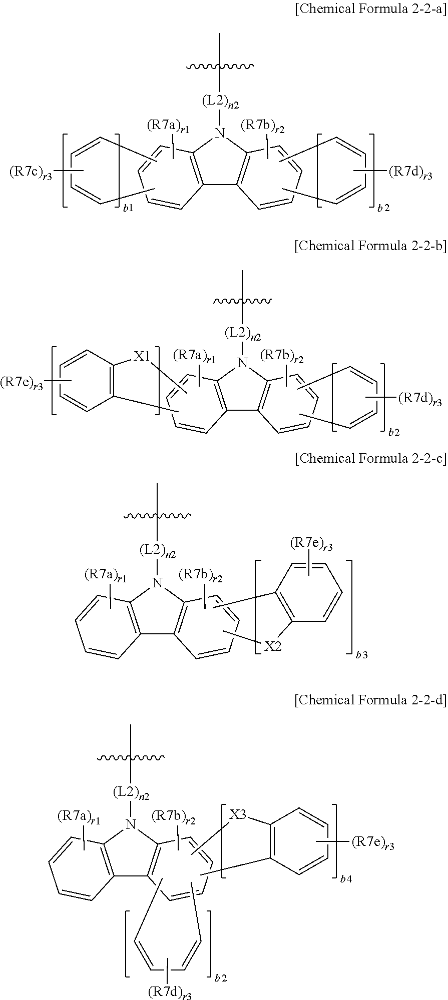

in Chemical Formulae 2-2-a to 2-2-d,
L2 and n2 have the same definitions as in Chemical Formula 1,
R7a to R7e are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group,
X1 to X3 are the same as or different from each other and each independently NR′, O or S, and herein, R′ is a substituted or unsubstituted C6 to C60 aryl group; or a substituted or unsubstituted C2 to C60 heteroaryl group,
b1, b2 and b3 are the same as or different from each other, and each independently an integer of 0 or 1,
b4 is an integer of 0, 1 or 2, however, when b4 is 2, b2 is 0, and
r1, r2 and r3 are the same as or different from each other, and each independently one of integers of 0 to 4.

In one embodiment of the present disclosure, R7a to R7e are the same as or different from each other, and may be each independently selected from the group consisting of hydrogen; deuterium; halogen; a cyano group; a substituted or unsubstituted C6 to C60 aryl group; and a substituted or unsubstituted C2 to C60 heteroaryl group.

In one embodiment of the present disclosure, R7a to R7e are the same as or different from each other, and may be each independently selected from the group consisting of hydrogen; deuterium; a substituted or unsubstituted C6 to C60 aryl group; and a substituted or unsubstituted C2 to C60 heteroaryl group.

In one embodiment of the present disclosure, R7a to R7e are the same as or different from each other, and may be each independently hydrogen; or deuterium.

In one embodiment of the present disclosure, X1 to X3 are the same as or different from each other and may be each independently NR′, O or S, and herein, R′ may be a substituted or unsubstituted C6 to C60 aryl group.

In one embodiment of the present disclosure, R′ may be a phenyl group.

In one embodiment of the present disclosure, in Chemical Formula 2-2-a, b1 and b2 may each be 0.

In one embodiment of the present disclosure, in Chemical Formula 2-2-a, b1 and b2 may each be 1.

In one embodiment of the present disclosure, in Chemical Formula 2-2-a, b1 may be 0, and b2 may be 1.

In one embodiment of the present disclosure, in Chemical Formula 2-2-a, b1 may be 1, and b2 may be 0.

In one embodiment of the present disclosure, in Chemical Formula 2-2-b, b2 and b3 may each be 0.

In one embodiment of the present disclosure, in Chemical Formula 2-2-b, b2 and b3 may each be 1.

In one embodiment of the present disclosure, in Chemical Formula 2-2-b, b2 may be 0, and b3 may be 1.

In one embodiment of the present disclosure, in Chemical Formula 2-2-c, b3 may be 0 or 1.

In one embodiment of the present disclosure, in Chemical Formula 2-2-d, b2 and b4 may each be 0.

In one embodiment of the present disclosure, in Chemical Formula 2-2-d, b2 and b4 may each be 1.

In one embodiment of the present disclosure, in Chemical Formula 2-2-d, b2 may be 0, and b4 may be 2.

In one embodiment of the present disclosure, the group represented by Chemical Formula 2-2 may include the following structural formulae, but is not limited to these examples:

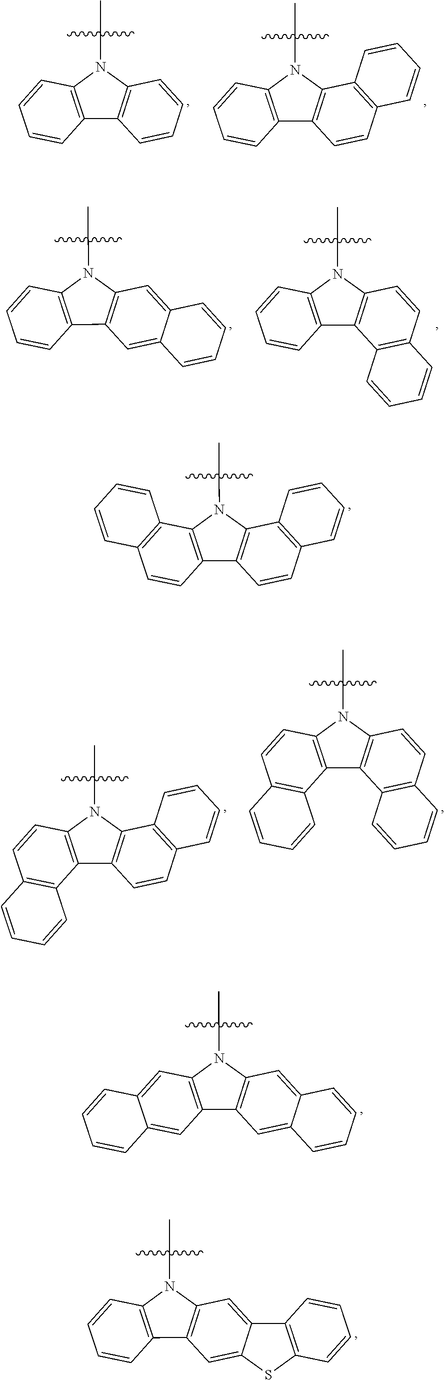

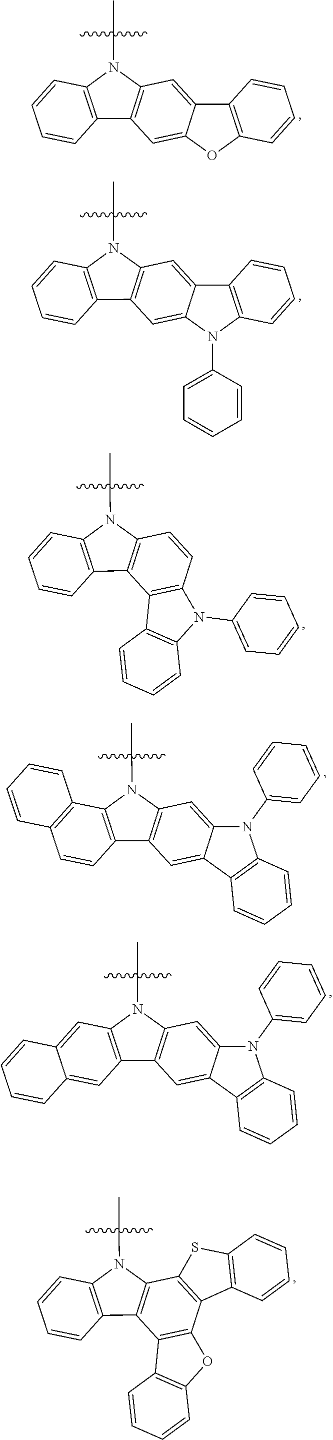

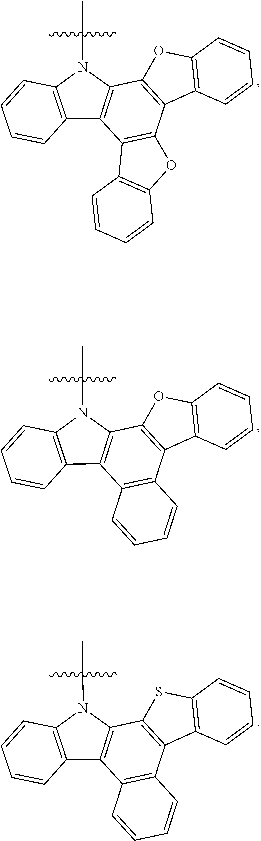 [SMILES: *n1c2ccccc2c2c3c4ccccc4oc3c3c4ccccc4oc3c21 ; *n1c2ccccc2c2c3ccccc3c3c4ccccc4oc3c21 ; *n1c2ccccc2c2c3ccccc3c3c4ccccc4sc3c21]

In one embodiment of the present disclosure, L1 and L2 are the same as or different from each other, and may be each independently a single bond; a substituted or unsubstituted C6 to C40 arylene group; or a substituted or unsubstituted C2 to C40 heteroarylene group.

In one embodiment of the present disclosure, L1 and L2 are the same as or different from each other, and may be each independently a single bond; a substituted or unsubstituted C6 to C20 arylene group; or a substituted or unsubstituted C2 to C20 heteroarylene group.

In one embodiment of the present disclosure, L1 and L2 are the same as or different from each other and may be each independently a single bond; a substituted or unsubstituted phenylene group; a substituted or unsubstituted biphenylene group; a substituted or unsubstituted terphenylene group; a substituted or unsubstituted naphthylene group; a substituted or unsubstituted fluorenylene group; a substituted or unsubstituted dibenzothiophenylene group; or a substituted or unsubstituted dibenzofuranylene group, but are not limited to these examples.

In the “substituted” phenylene group, the “substituted” biphenylene group, the “substituted” terphenylene group, the “substituted” naphthylene group, the “substituted” fluorenylene group, the “substituted” dibenzothiophenylene group and the “substituted” dibenzofuranylene group, the “substituted” may refer to being substituted with a halogen group; a cyano group; a methyl group; a trifluoromethyl group; or a tert-butyl group. For example, the “substituted” fluorenylene group may include a 9,9-dimethylfluorenylene group. However, the substituted groups are not limited to these examples.

In one embodiment of the present disclosure, n1 and n2 are the same as or different from each other, and each independently one of integers of 0 to 3, and when n1 and n2 are 2 or greater, L1s or L2s are the same as or different from each other.

In one embodiment of the present disclosure, the heterocyclic compound represented by Chemical Formula 1 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less, with respect to the total number of hydrogen atoms and deuterium atoms.

In one embodiment of the present disclosure, the heterocyclic compound represented by Chemical Formula 1 may not include deuterium, or may have a deuterium content of 1% to 100% with respect to the total number of hydrogen atoms and deuterium atoms.

In one embodiment of the present disclosure, the heterocyclic compound represented by Chemical Formula 1 may not include deuterium, or may have a deuterium content of 20% to 90% with respect to the total number of hydrogen atoms and deuterium atoms.

In one embodiment of the present disclosure, the heterocyclic compound represented by Chemical Formula 1 may not include deuterium, or may have a deuterium content of 30% to 80% with respect to the total number of hydrogen atoms and deuterium atoms.

In one embodiment of the present disclosure, the heterocyclic compound represented by Chemical Formula 1 may not include deuterium, or may have a deuterium content of 40% to 70% with respect to the total number of hydrogen atoms and deuterium atoms.

For example, the heterocyclic compound represented by Chemical Formula 1 may have a deuterium content of 0% or greater, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms.

In one embodiment of the present disclosure, the heterocyclic compound represented by Chemical Formula 1 may be any one selected from the group consisting of the following compounds:

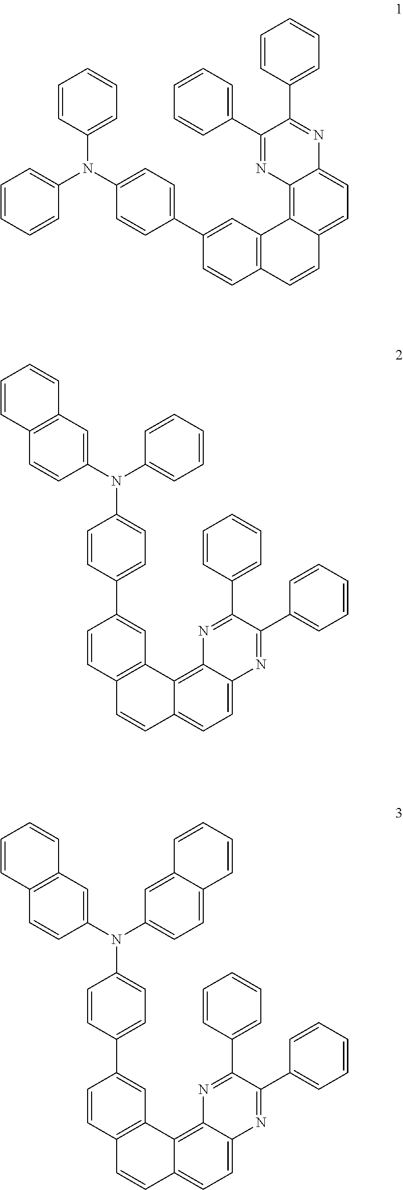 [SMILES: c1ccc(-c2nc3ccc4ccc5ccc(-c6ccc(N(c7ccccc7)c7ccccc7)cc6)cc5c4c3nc2-c2ccccc2)cc1 ; c1ccc(-c2nc3ccc4ccc5ccc(-c6ccc(N(c7ccccc7)c7ccc8ccccc8c7)cc6)cc5c4c3nc2-c2ccccc2)cc1 ; c1ccc(-c2nc3ccc4ccc5ccc(-c6ccc(N(c7ccc8ccccc8c7)c7ccc8ccccc8c7)cc6)cc5c4c3nc2-c2ccccc2)cc1]

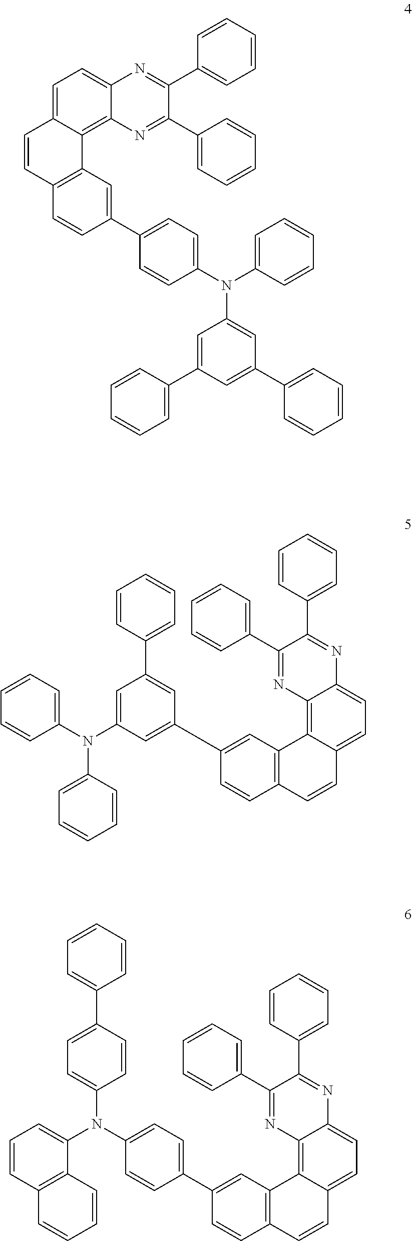 [SMILES: c1ccc(-c2cc(-c3ccccc3)cc(N(c3ccccc3)c3ccc(-c4ccc5ccc6ccc7nc(-c8ccccc8)c(-c8ccccc8)nc7c6c5c4)cc3)c2)cc1 ; c1ccc(-c2cc(-c3ccc4ccc5ccc6nc(-c7ccccc7)c(-c7ccccc7)nc6c5c4c3)cc(N(c3ccccc3)c3ccccc3)c2)cc1 ; c1ccc(-c2ccc(N(c3ccc(-c4ccc5ccc6ccc7nc(-c8ccccc8)c(-c8ccccc8)nc7c6c5c4)cc3)c3cccc4ccccc34)cc2)cc1]

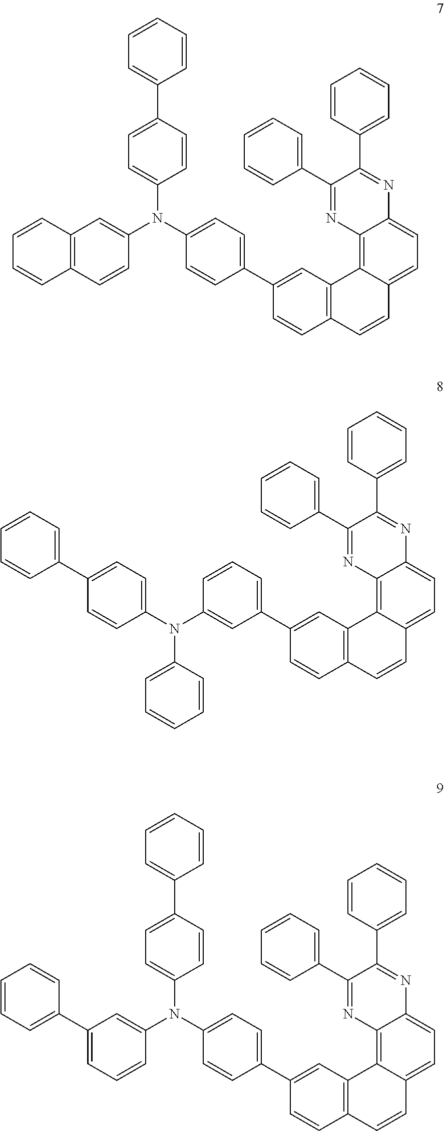 [SMILES: c1ccc(-c2ccc(N(c3ccc(-c4ccc5ccc6ccc7nc(-c8ccccc8)c(-c8ccccc8)nc7c6c5c4)cc3)c3ccc4ccccc4c3)cc2)cc1 ; c1ccc(-c2ccc(N(c3ccccc3)c3cccc(-c4ccc5ccc6ccc7nc(-c8ccccc8)c(-c8ccccc8)nc7c6c5c4)c3)cc2)cc1 ; c1ccc(-c2ccc(N(c3ccc(-c4ccc5ccc6ccc7nc(-c8ccccc8)c(-c8ccccc8)nc7c6c5c4)cc3)c3cccc(-c4ccccc4)c3)cc2)cc1]

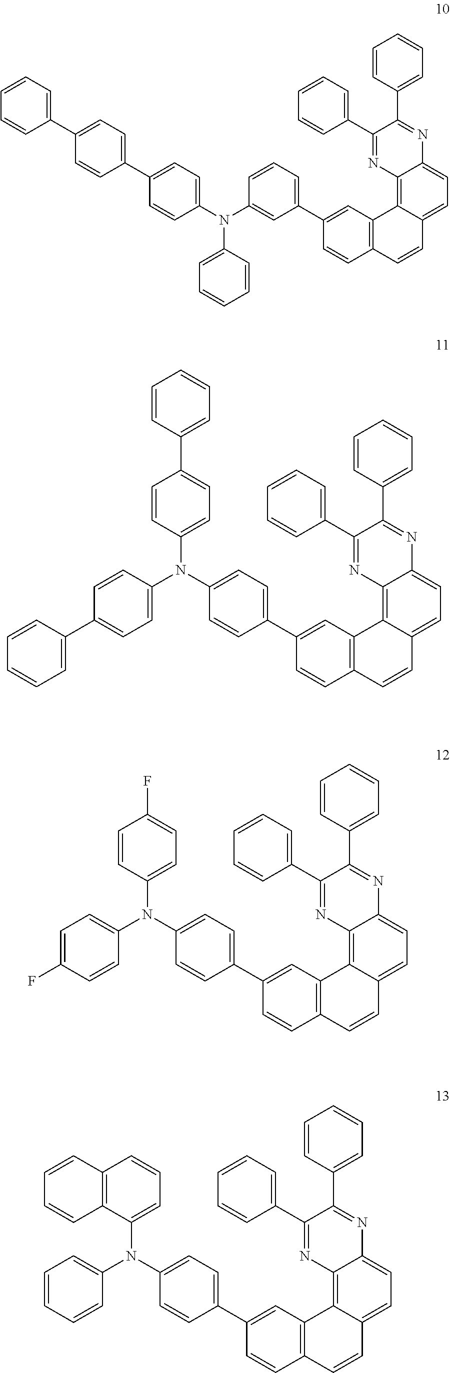 [SMILES: c1ccc(-c2ccc(-c3ccc(N(c4ccccc4)c4cccc(-c5ccc6ccc7ccc8nc(-c9ccccc9)c(-c9ccccc9)nc8c7c6c5)c4)cc3)cc2)cc1 ; c1ccc(-c2ccc(N(c3ccc(-c4ccccc4)cc3)c3ccc(-c4ccc5ccc6ccc7nc(-c8ccccc8)c(-c8ccccc8)nc7c6c5c4)cc3)cc2)cc1 ; Fc1ccc(N(c2ccc(F)cc2)c2ccc(-c3ccc4ccc5ccc6nc(-c7ccccc7)c(-c7ccccc7)nc6c5c4c3)cc2)cc1 ; c1ccc(-c2nc3ccc4ccc5ccc(-c6ccc(N(c7ccccc7)c7cccc8ccccc78)cc6)cc5c4c3nc2-c2ccccc2)cc1]

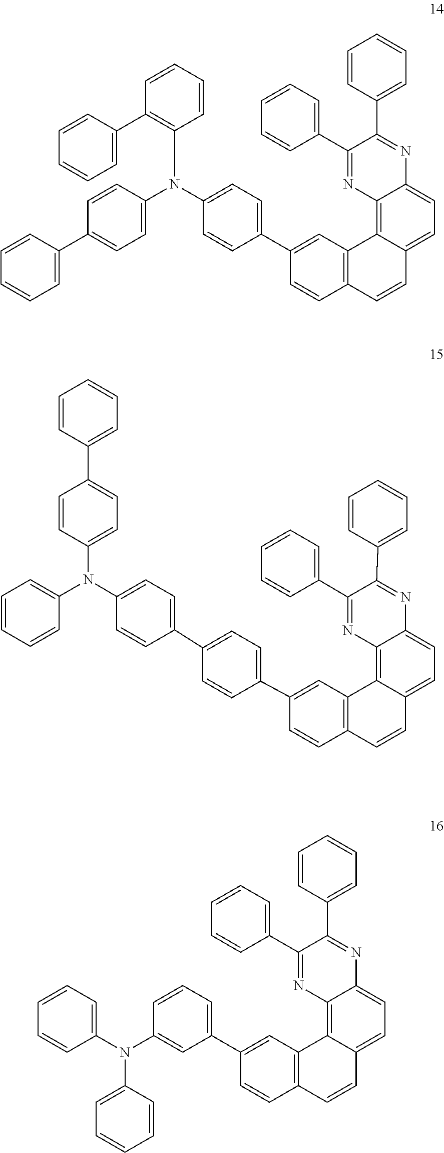 [SMILES: c1ccc(-c2ccc(N(c3ccc(-c4ccc5ccc6ccc7nc(-c8ccccc8)c(-c8ccccc8)nc7c6c5c4)cc3)c3ccccc3-c3ccccc3)cc2)cc1 ; c1ccc(-c2ccc(N(c3ccccc3)c3ccc(-c4ccc(-c5ccc6ccc7ccc8nc(-c9ccccc9)c(-c9ccccc9)nc8c7c6c5)cc4)cc3)cc2)cc1 ; c1ccc(-c2nc3ccc4ccc5ccc(-c6cccc(N(c7ccccc7)c7ccccc7)c6)cc5c4c3nc2-c2ccccc2)cc1]

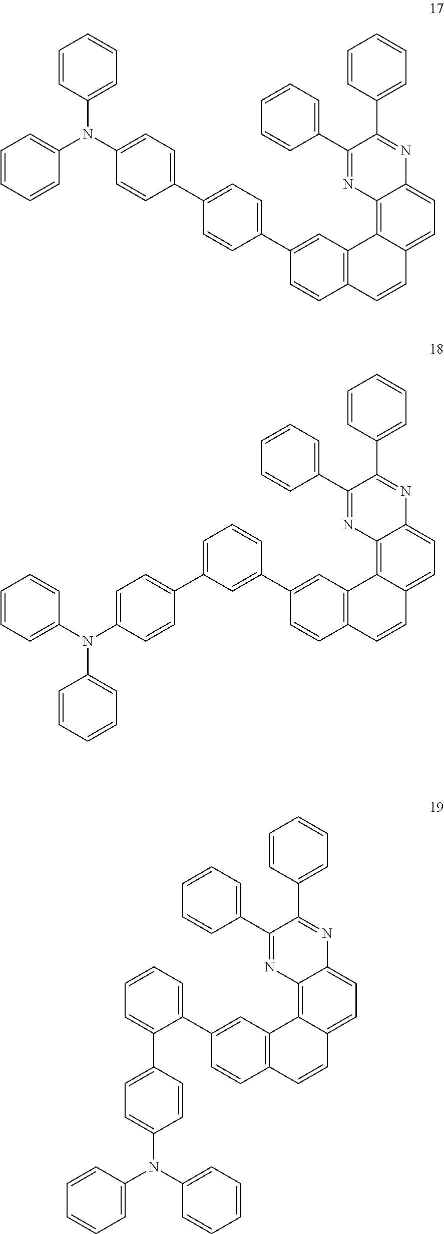 [SMILES: c1ccc(-c2nc3ccc4ccc5ccc(-c6ccc(-c7ccc(N(c8ccccc8)c8ccccc8)cc7)cc6)cc5c4c3nc2-c2ccccc2)cc1 ; c1ccc(-c2nc3ccc4ccc5ccc(-c6cccc(-c7ccc(N(c8ccccc8)c8ccccc8)cc7)c6)cc5c4c3nc2-c2ccccc2)cc1 ; c1ccc(-c2nc3ccc4ccc5ccc(-c6ccccc6-c6ccc(N(c7ccccc7)c7ccccc7)cc6)cc5c4c3nc2-c2ccccc2)cc1]

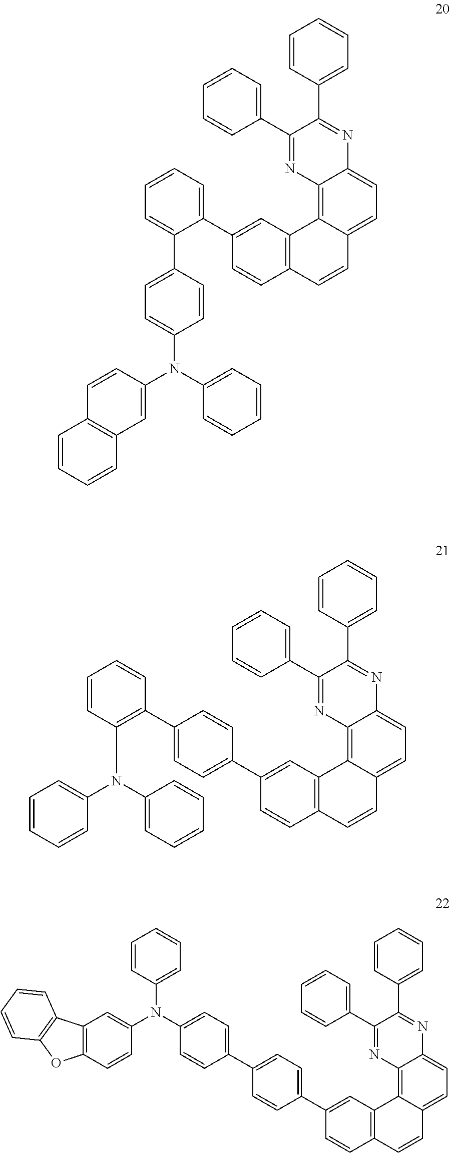 [SMILES: c1ccc(-c2nc3ccc4ccc5ccc(-c6ccccc6-c6ccc(N(c7ccccc7)c7ccc8ccccc8c7)cc6)cc5c4c3nc2-c2ccccc2)cc1 ; c1ccc(-c2nc3ccc4ccc5ccc(-c6ccc(-c7ccccc7N(c7ccccc7)c7ccccc7)cc6)cc5c4c3nc2-c2ccccc2)cc1 ; c1ccc(-c2nc3ccc4ccc5ccc(-c6ccc(-c7ccc(N(c8ccccc8)c8ccc9oc%10ccccc%10c9c8)cc7)cc6)cc5c4c3nc2-c2ccccc2)cc1]

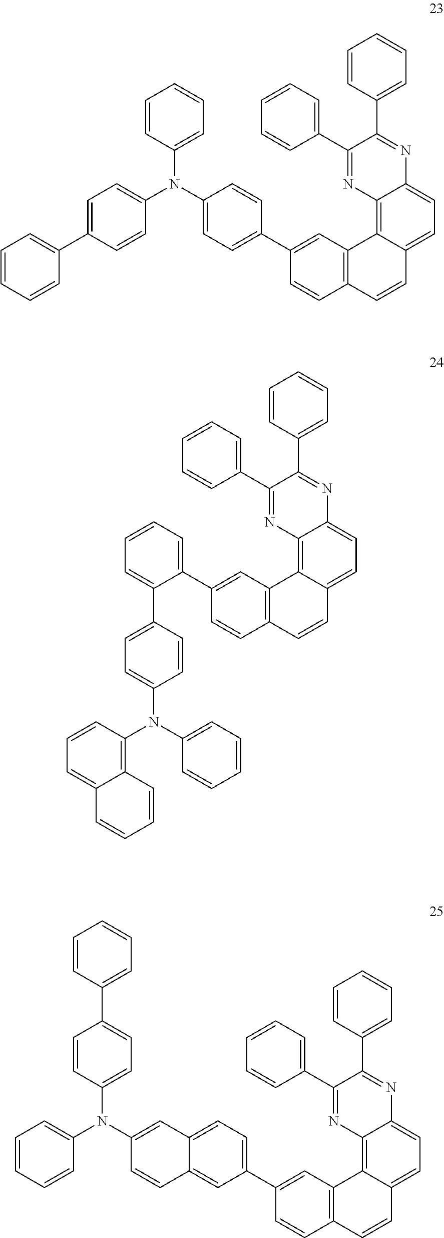 [SMILES: c1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; c1ccc(-c2nc3ccc4ccc5ccc(-c6ccccc6-c6ccc(N(c7ccccc7)c7cccc8ccccc78)cc6)cc5c4c3nc2-c2ccccc2)cc1 ; c1ccc(-c2ccc(N(c3ccccc3)c3ccc4cc(-c5ccc6ccc7ccc8nc(-c9ccccc9)c(-c9ccccc9)nc8c7c6c5)ccc4c3)cc2)cc1]

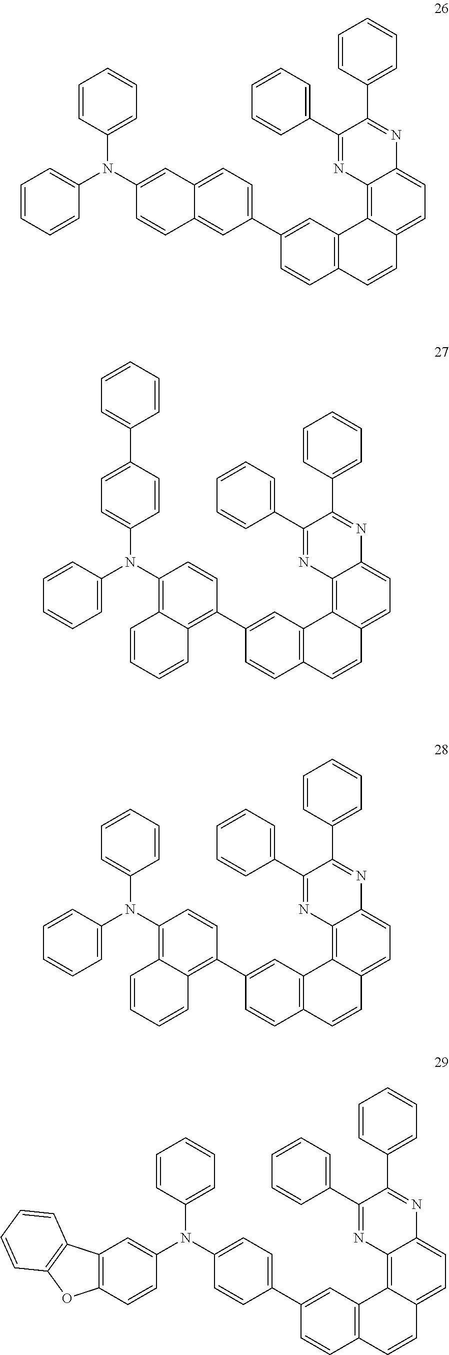 [SMILES: c1ccc(-c2nc3ccc4ccc5ccc(-c6ccc7cc(N(c8ccccc8)c8ccccc8)ccc7c6)cc5c4c3nc2-c2ccccc2)cc1 ; c1ccc(-c2ccc(N(c3ccccc3)c3ccc(-c4ccc5ccc6ccc7nc(-c8ccccc8)c(-c8ccccc8)nc7c6c5c4)c4ccccc34)cc2)cc1 ; c1ccc(-c2nc3ccc4ccc5ccc(-c6ccc(N(c7ccccc7)c7ccccc7)c7ccccc67)cc5c4c3nc2-c2ccccc2)cc1 ; c1ccc(-c2nc3ccc4ccc5ccc(-c6ccc(N(c7ccccc7)c7ccc8oc9ccccc9c8c7)cc6)cc5c4c3nc2-c2ccccc2)cc1]

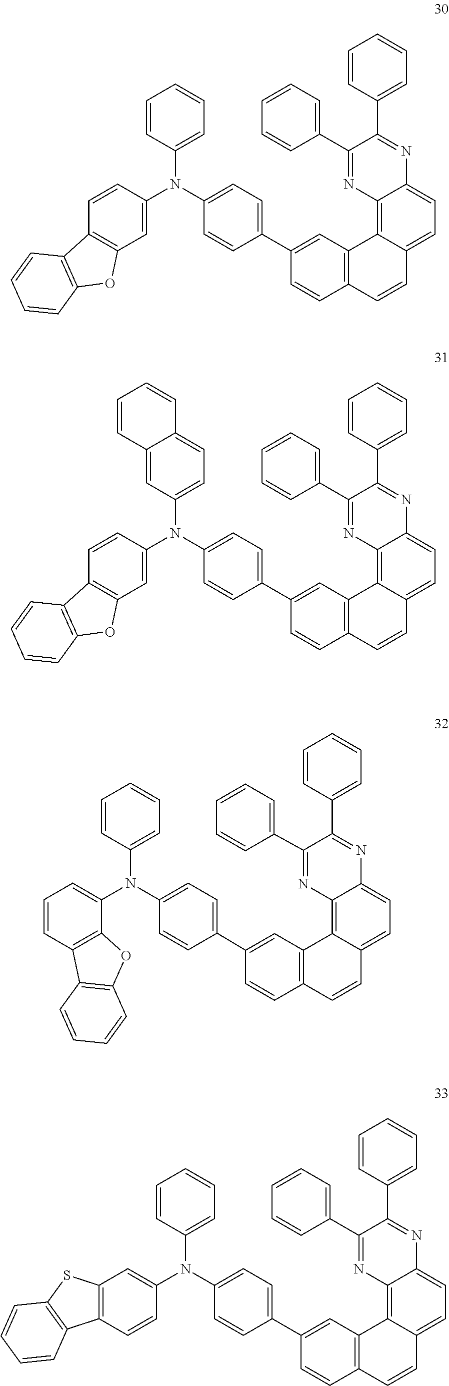 [SMILES: c1ccc(-c2nc3ccc4ccc5ccc(-c6ccc(N(c7ccccc7)c7ccc8c(c7)oc7ccccc78)cc6)cc5c4c3nc2-c2ccccc2)cc1 ; c1ccc(-c2nc3ccc4ccc5ccc(-c6ccc(N(c7ccc8ccccc8c7)c7ccc8c(c7)oc7ccccc78)cc6)cc5c4c3nc2-c2ccccc2)cc1 ; c1ccc(-c2nc3ccc4ccc5ccc(-c6ccc(N(c7ccccc7)c7cccc8c7oc7ccccc78)cc6)cc5c4c3nc2-c2ccccc2)cc1 ; c1ccc(-c2nc3ccc4ccc5ccc(-c6ccc(N(c7ccccc7)c7ccc8c(c7)sc7ccccc78)cc6)cc5c4c3nc2-c2ccccc2)cc1]

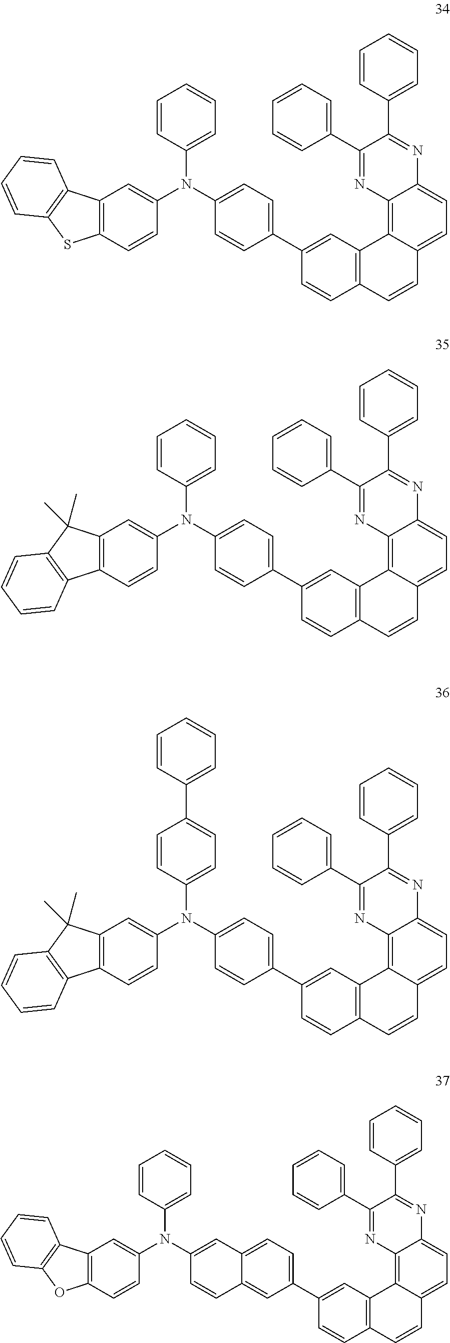 [SMILES: c1ccc(-c2nc3ccc4ccc5ccc(-c6ccc(N(c7ccccc7)c7ccc8sc9ccccc9c8c7)cc6)cc5c4c3nc2-c2ccccc2)cc1 ; CC1(C)c2ccccc2-c2ccc(N(c3ccccc3)c3ccc(-c4ccc5ccc6ccc7nc(-c8ccccc8)c(-c8ccccc8)nc7c6c5c4)cc3)cc21 ; CC1(C)c2ccccc2-c2ccc(N(c3ccc(-c4ccccc4)cc3)c3ccc(-c4ccc5ccc6ccc7nc(-c8ccccc8)c(-c8ccccc8)nc7c6c5c4)cc3)cc21 ; c1ccc(-c2nc3ccc4ccc5ccc(-c6ccc7cc(N(c8ccccc8)c8ccc9oc%10ccccc%10c9c8)ccc7c6)cc5c4c3nc2-c2ccccc2)cc1]

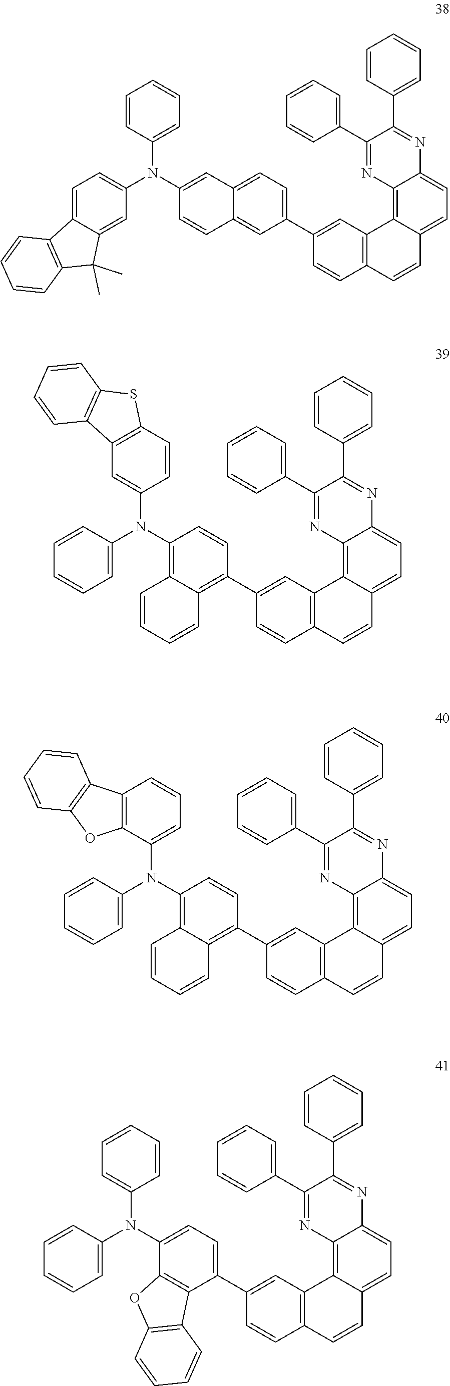 [SMILES: CC1(C)c2ccccc2-c2ccc(N(c3ccccc3)c3ccc4cc(-c5ccc6ccc7ccc8nc(-c9ccccc9)c(-c9ccccc9)nc8c7c6c5)ccc4c3)cc21 ; c1ccc(-c2nc3ccc4ccc5ccc(-c6ccc(N(c7ccccc7)c7ccc8sc9ccccc9c8c7)c7ccccc67)cc5c4c3nc2-c2ccccc2)cc1 ; c1ccc(-c2nc3ccc4ccc5ccc(-c6ccc(N(c7ccccc7)c7cccc8c7oc7ccccc78)c7ccccc67)cc5c4c3nc2-c2ccccc2)cc1 ; c1ccc(-c2nc3ccc4ccc5ccc(-c6ccc(N(c7ccccc7)c7ccccc7)c7oc8ccccc8c67)cc5c4c3nc2-c2ccccc2)cc1]

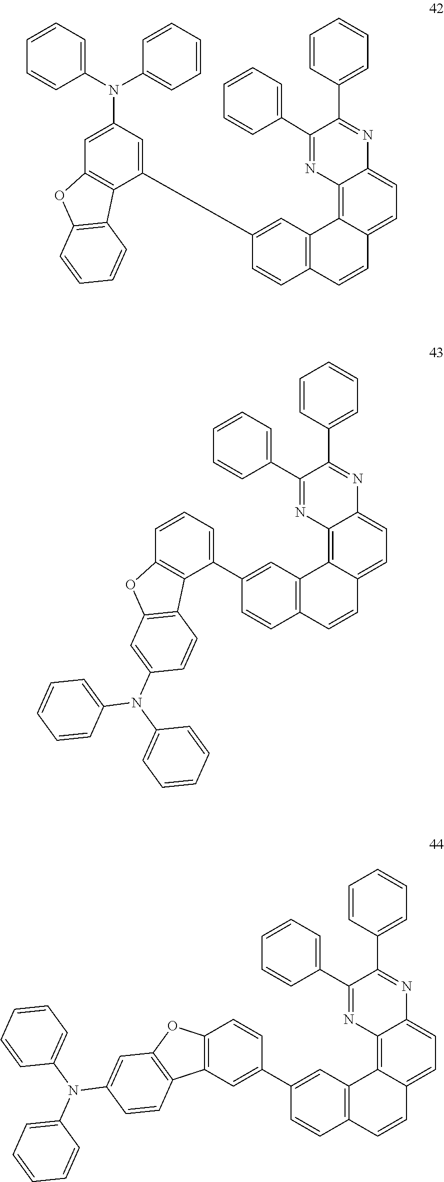 [SMILES: c1ccc(-c2nc3ccc4ccc5ccc(-c6cc(N(c7ccccc7)c7ccccc7)cc7oc8ccccc8c67)cc5c4c3nc2-c2ccccc2)cc1 ; c1ccc(-c2nc3ccc4ccc5ccc(-c6cccc7oc8cc(N(c9ccccc9)c9ccccc9)ccc8c67)cc5c4c3nc2-c2ccccc2)cc1 ; c1ccc(-c2nc3ccc4ccc5ccc(-c6ccc7oc8cc(N(c9ccccc9)c9ccccc9)ccc8c7c6)cc5c4c3nc2-c2ccccc2)cc1]

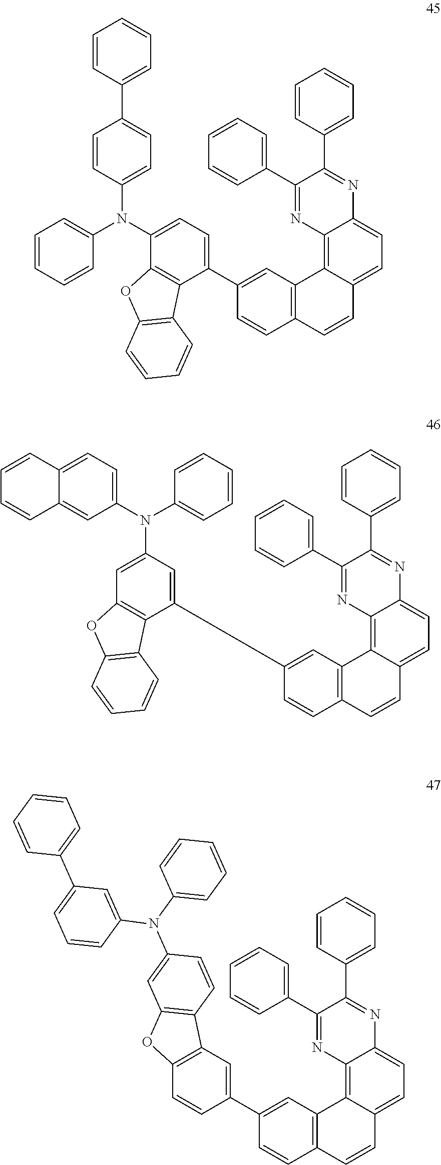 [SMILES: c1ccc(-c2ccc(N(c3ccccc3)c3ccc(-c4ccc5ccc6ccc7nc(-c8ccccc8)c(-c8ccccc8)nc7c6c5c4)c4c3oc3ccccc34)cc2)cc1 ; c1ccc(-c2nc3ccc4ccc5ccc(-c6cc(N(c7ccccc7)c7ccc8ccccc8c7)cc7oc8ccccc8c67)cc5c4c3nc2-c2ccccc2)cc1 ; c1ccc(-c2cccc(N(c3ccccc3)c3ccc4c(c3)oc3ccc(-c5ccc6ccc7ccc8nc(-c9ccccc9)c(-c9ccccc9)nc8c7c6c5)cc34)c2)cc1]

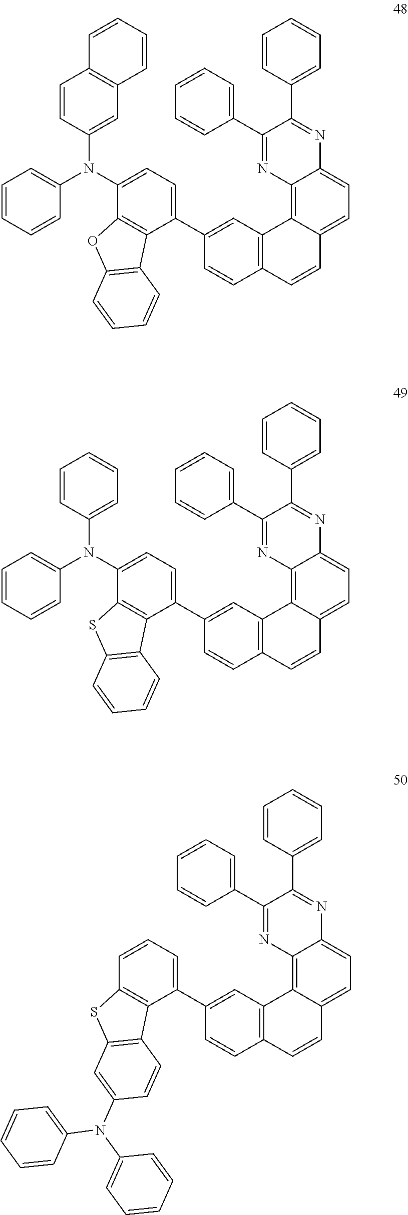 [SMILES: c1ccc(-c2nc3ccc4ccc5ccc(-c6ccc(N(c7ccccc7)c7ccc8ccccc8c7)c7oc8ccccc8c67)cc5c4c3nc2-c2ccccc2)cc1 ; c1ccc(-c2nc3ccc4ccc5ccc(-c6ccc(N(c7ccccc7)c7ccccc7)c7sc8ccccc8c67)cc5c4c3nc2-c2ccccc2)cc1 ; c1ccc(-c2nc3ccc4ccc5ccc(-c6cccc7sc8cc(N(c9ccccc9)c9ccccc9)ccc8c67)cc5c4c3nc2-c2ccccc2)cc1]

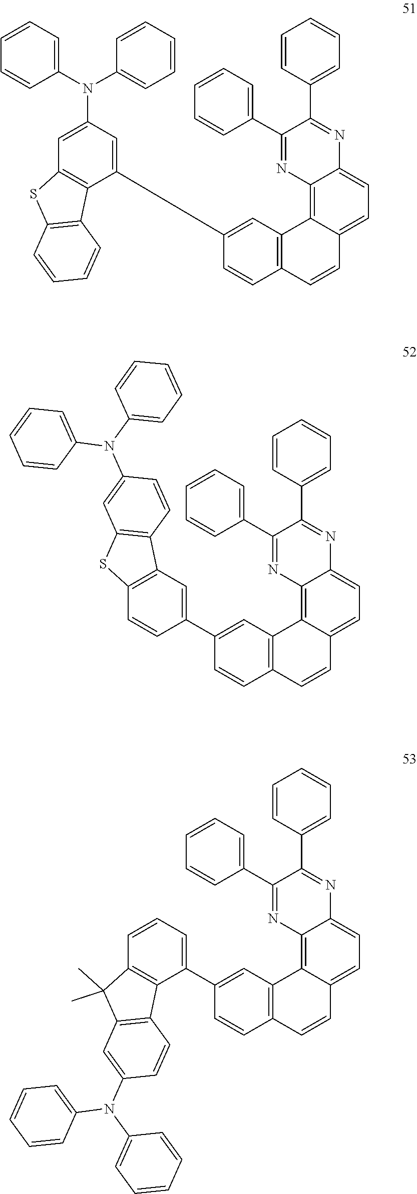 [SMILES: c1ccc(-c2nc3ccc4ccc5ccc(-c6cc(N(c7ccccc7)c7ccccc7)cc7sc8ccccc8c67)cc5c4c3nc2-c2ccccc2)cc1 ; c1ccc(-c2nc3ccc4ccc5ccc(-c6ccc7sc8cc(N(c9ccccc9)c9ccccc9)ccc8c7c6)cc5c4c3nc2-c2ccccc2)cc1 ; CC1(C)c2cc(N(c3ccccc3)c3ccccc3)ccc2-c2c(-c3ccc4ccc5ccc6nc(-c7ccccc7)c(-c7ccccc7)nc6c5c4c3)cccc21]

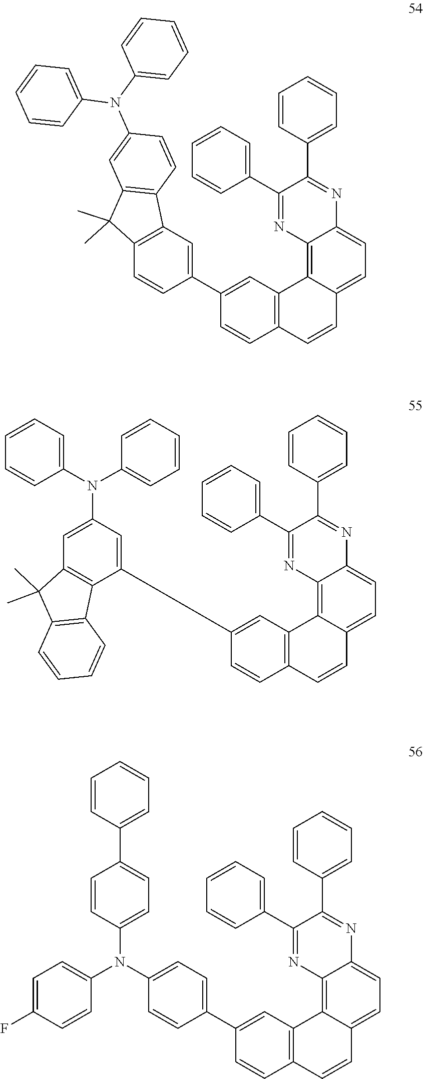 [SMILES: CC1(C)c2ccc(-c3ccc4ccc5ccc6nc(-c7ccccc7)c(-c7ccccc7)nc6c5c4c3)cc2-c2ccc(N(c3ccccc3)c3ccccc3)cc21 ; CC1(C)c2ccccc2-c2c(-c3ccc4ccc5ccc6nc(-c7ccccc7)c(-c7ccccc7)nc6c5c4c3)cc(N(c3ccccc3)c3ccccc3)cc21 ; Fc1ccc(N(c2ccc(-c3ccccc3)cc2)c2ccc(-c3ccc4ccc5ccc6nc(-c7ccccc7)c(-c7ccccc7)nc6c5c4c3)cc2)cc1]

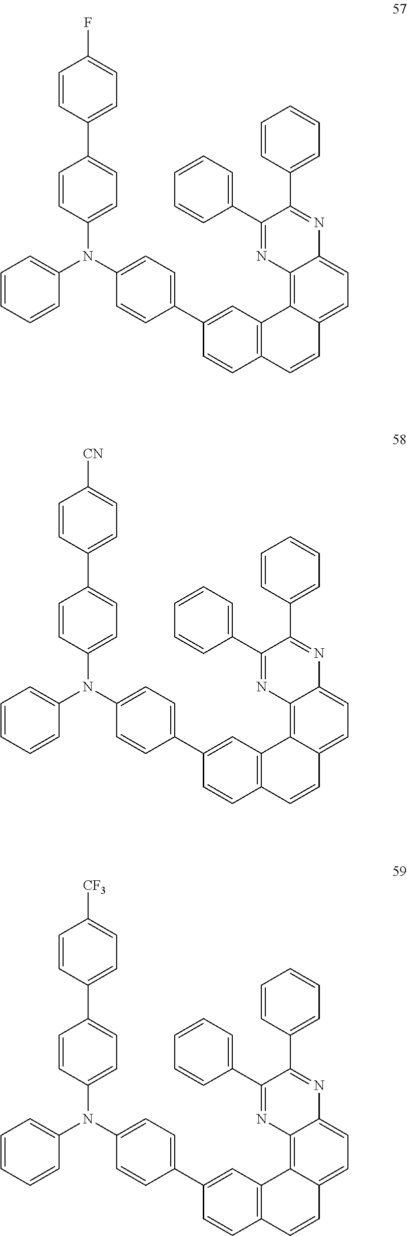 [SMILES: Fc1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; N#Cc1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; FC(F)(F)c1ccc(-c2ccc(N(c3ccccc3)c3ccc(-c4ccc5ccc6ccc7nc(-c8ccccc8)c(-c8ccccc8)nc7c6c5c4)cc3)cc2)cc1]

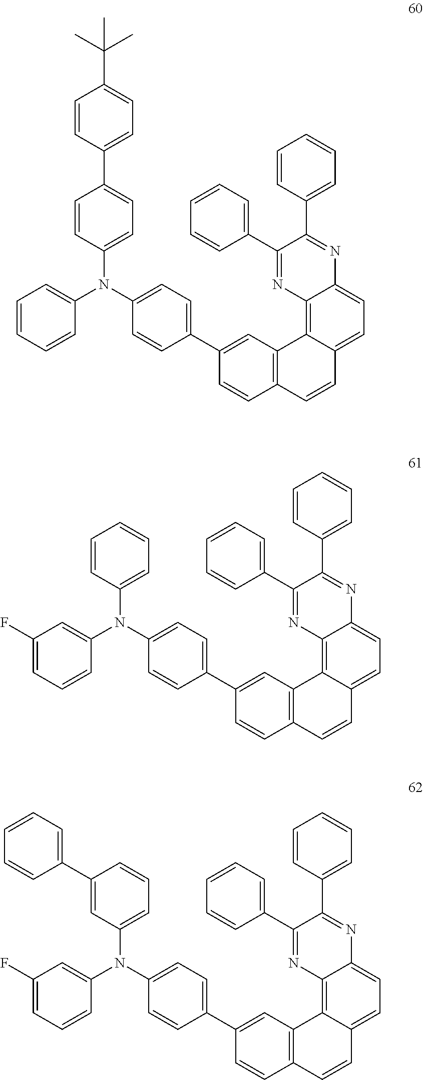 [SMILES: CC(C)(C)c1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; Fc1cccc(N(c2ccccc2)c2ccc(-c3ccc4ccc5ccc6nc(-c7ccccc7)c(-c7ccccc7)nc6c5c4c3)cc2)c1 ; Fc1cccc(N(c2ccc(-c3ccc4ccc5ccc6nc(-c7ccccc7)c(-c7ccccc7)nc6c5c4c3)cc2)c2cccc(-c3ccccc3)c2)c1]

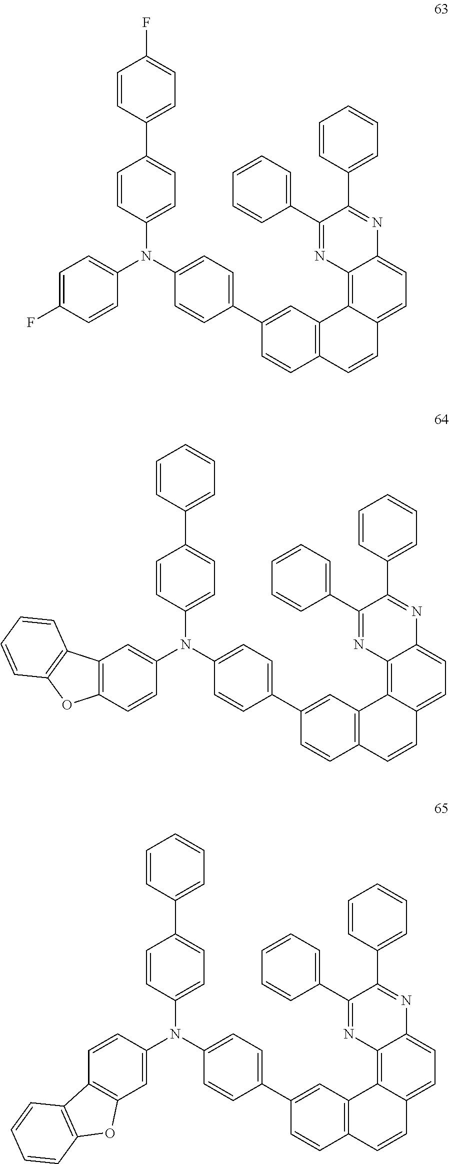 [SMILES: Fc1ccc(-c2ccc(N(c3ccc(F)cc3)c3ccc(-c4ccc5ccc6ccc7nc(-c8ccccc8)c(-c8ccccc8)nc7c6c5c4)cc3)cc2)cc1 ; c1ccc(-c2ccc(N(c3ccc(-c4ccc5ccc6ccc7nc(-c8ccccc8)c(-c8ccccc8)nc7c6c5c4)cc3)c3ccc4oc5ccccc5c4c3)cc2)cc1 ; c1ccc(-c2ccc(N(c3ccc(-c4ccc5ccc6ccc7nc(-c8ccccc8)c(-c8ccccc8)nc7c6c5c4)cc3)c3ccc4c(c3)oc3ccccc34)cc2)cc1]

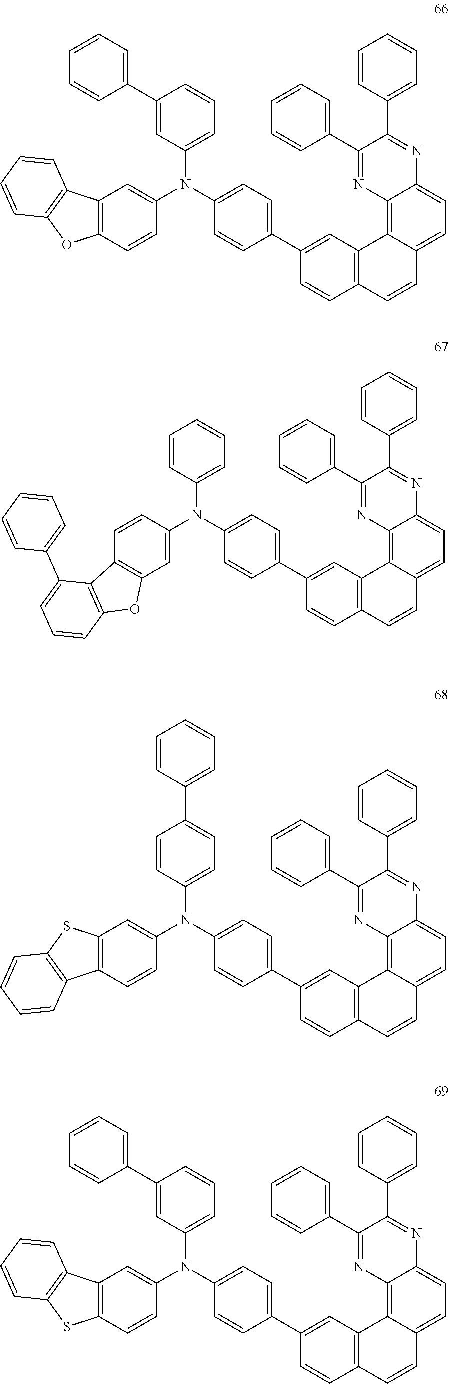 [SMILES: c1ccc(-c2cccc(N(c3ccc(-c4ccc5ccc6ccc7nc(-c8ccccc8)c(-c8ccccc8)nc7c6c5c4)cc3)c3ccc4oc5ccccc5c4c3)c2)cc1 ; c1ccc(-c2nc3ccc4ccc5ccc(-c6ccc(N(c7ccccc7)c7ccc8c(c7)oc7cccc(-c9ccccc9)c78)cc6)cc5c4c3nc2-c2ccccc2)cc1 ; c1ccc(-c2ccc(N(c3ccc(-c4ccc5ccc6ccc7nc(-c8ccccc8)c(-c8ccccc8)nc7c6c5c4)cc3)c3ccc4c(c3)sc3ccccc34)cc2)cc1 ; c1ccc(-c2cccc(N(c3ccc(-c4ccc5ccc6ccc7nc(-c8ccccc8)c(-c8ccccc8)nc7c6c5c4)cc3)c3ccc4sc5ccccc5c4c3)c2)cc1]

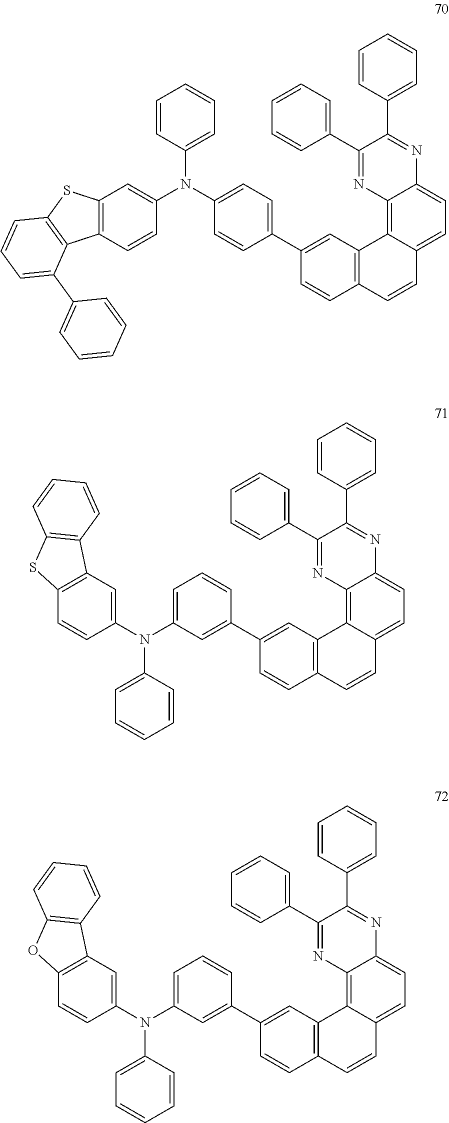 [SMILES: c1ccc(-c2nc3ccc4ccc5ccc(-c6ccc(N(c7ccccc7)c7ccc8c(c7)sc7cccc(-c9ccccc9)c78)cc6)cc5c4c3nc2-c2ccccc2)cc1 ; c1ccc(-c2nc3ccc4ccc5ccc(-c6cccc(N(c7ccccc7)c7ccc8sc9ccccc9c8c7)c6)cc5c4c3nc2-c2ccccc2)cc1 ; c1ccc(-c2nc3ccc4ccc5ccc(-c6cccc(N(c7ccccc7)c7ccc8oc9ccccc9c8c7)c6)cc5c4c3nc2-c2ccccc2)cc1]

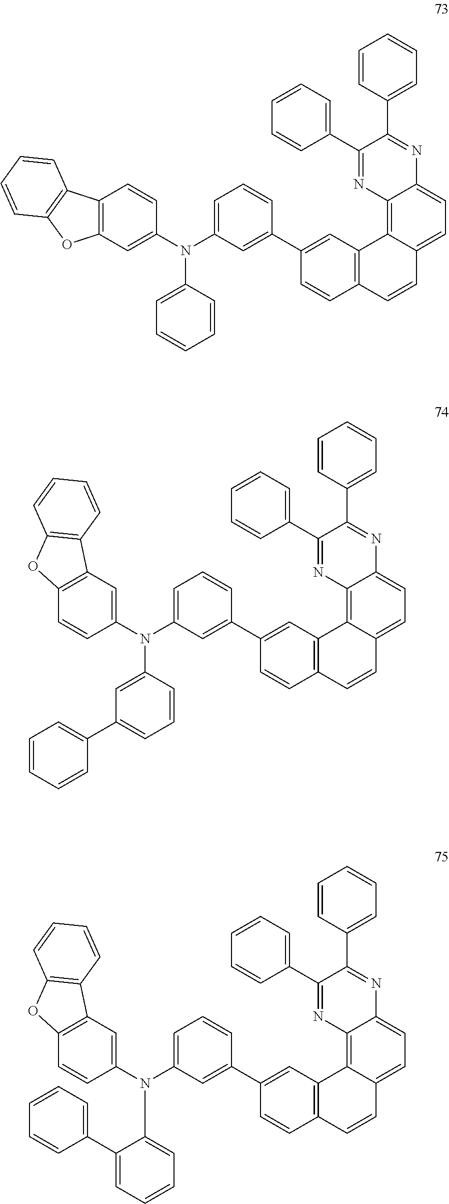 [SMILES: c1ccc(-c2nc3ccc4ccc5ccc(-c6cccc(N(c7ccccc7)c7ccc8c(c7)oc7ccccc78)c6)cc5c4c3nc2-c2ccccc2)cc1 ; c1ccc(-c2cccc(N(c3cccc(-c4ccc5ccc6ccc7nc(-c8ccccc8)c(-c8ccccc8)nc7c6c5c4)c3)c3ccc4oc5ccccc5c4c3)c2)cc1 ; c1ccc(-c2ccccc2N(c2cccc(-c3ccc4ccc5ccc6nc(-c7ccccc7)c(-c7ccccc7)nc6c5c4c3)c2)c2ccc3oc4ccccc4c3c2)cc1]

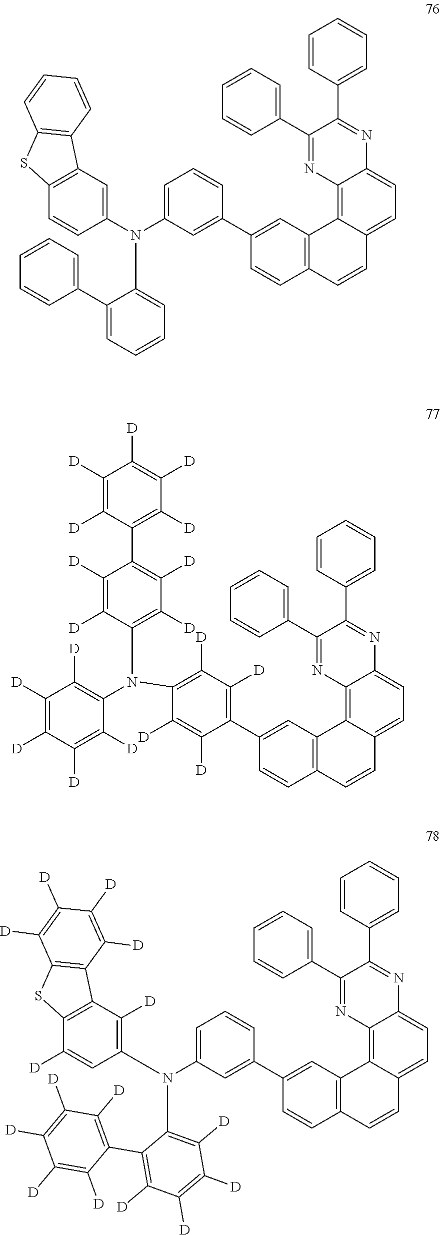 [SMILES: c1ccc(-c2ccccc2N(c2cccc(-c3ccc4ccc5ccc6nc(-c7ccccc7)c(-c7ccccc7)nc6c5c4c3)c2)c2ccc3sc4ccccc4c3c2)cc1 ; [2H]c1c([2H])c([2H])c(-c2c([2H])c([2H])c(N(c3c([2H])c([2H])c([2H])c([2H])c3[2H])c3c([2H])c([2H])c(-c4ccc5ccc6ccc7nc(-c8ccccc8)c(-c8ccccc8)nc7c6c5c4)c([2H])c3[2H])c([2H])c2[2H])c([2H])c1[2H] ; [2H]c1c([2H])c([2H])c(-c2c([2H])c([2H])c([2H])c([2H])c2N(c2cccc(-c3ccc4ccc5ccc6nc(-c7ccccc7)c(-c7ccccc7)nc6c5c4c3)c2)c2cc([2H])c3sc4c([2H])c([2H])c([2H])c([2H])c4c3c2[2H])c([2H])c1[2H]]

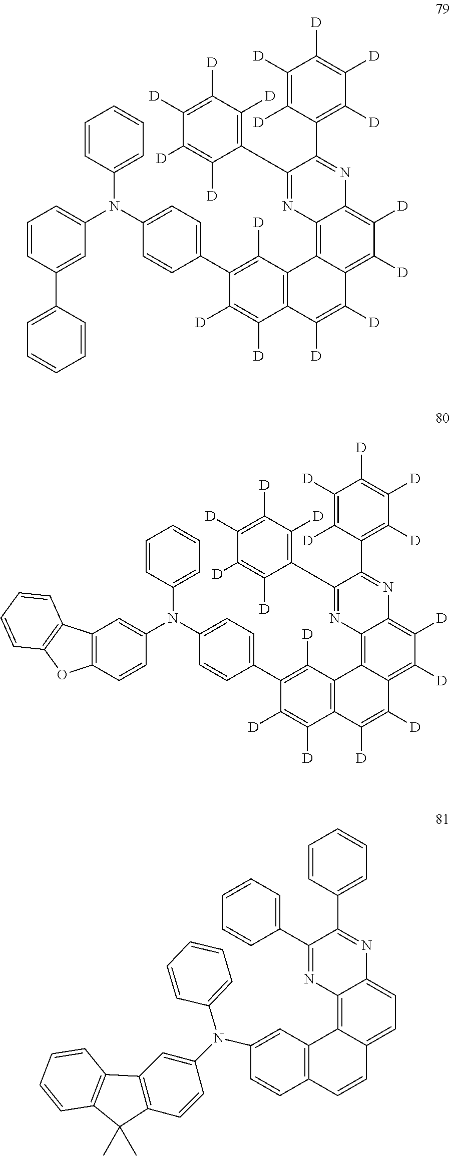 [SMILES: [2H]c1c([2H])c([2H])c(-c2nc3c([2H])c([2H])c4c([2H])c([2H])c5c([2H])c([2H])c(-c6ccc(N(c7ccccc7)c7cccc(-c8ccccc8)c7)cc6)c([2H])c5c4c3nc2-c2c([2H])c([2H])c([2H])c([2H])c2[2H])c([2H])c1[2H] ; [2H]c1c([2H])c([2H])c(-c2nc3c([2H])c([2H])c4c([2H])c([2H])c5c([2H])c([2H])c(-c6ccc(N(c7ccccc7)c7ccc8oc9ccccc9c8c7)cc6)c([2H])c5c4c3nc2-c2c([2H])c([2H])c([2H])c([2H])c2[2H])c([2H])c1[2H] ; CC1(C)c2ccccc2-c2cc(N(c3ccccc3)c3ccc4ccc5ccc6nc(-c7ccccc7)c(-c7ccccc7)nc6c5c4c3)ccc21]

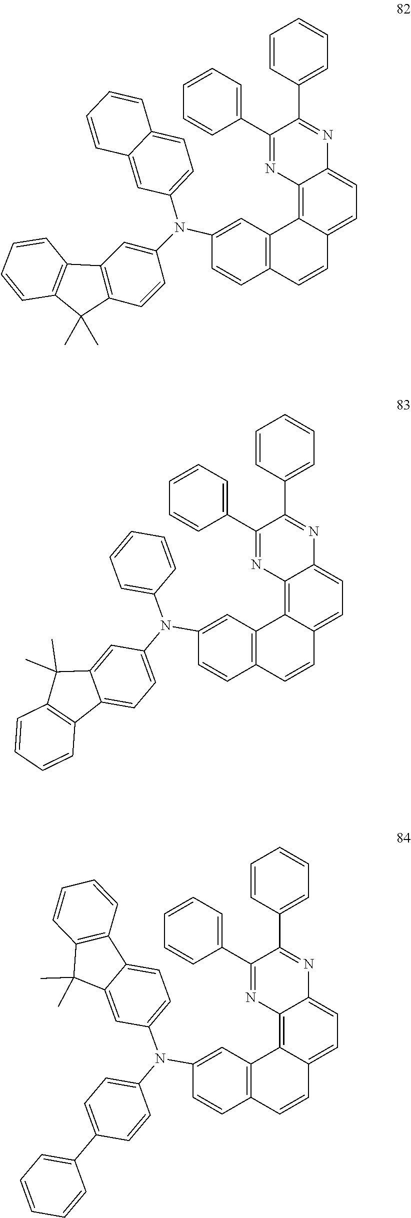 [SMILES: CC1(C)c2ccccc2-c2cc(N(c3ccc4ccccc4c3)c3ccc4ccc5ccc6nc(-c7ccccc7)c(-c7ccccc7)nc6c5c4c3)ccc21 ; CC1(C)c2ccccc2-c2ccc(N(c3ccccc3)c3ccc4ccc5ccc6nc(-c7ccccc7)c(-c7ccccc7)nc6c5c4c3)cc21 ; CC1(C)c2ccccc2-c2ccc(N(c3ccc(-c4ccccc4)cc3)c3ccc4ccc5ccc6nc(-c7ccccc7)c(-c7ccccc7)nc6c5c4c3)cc21]

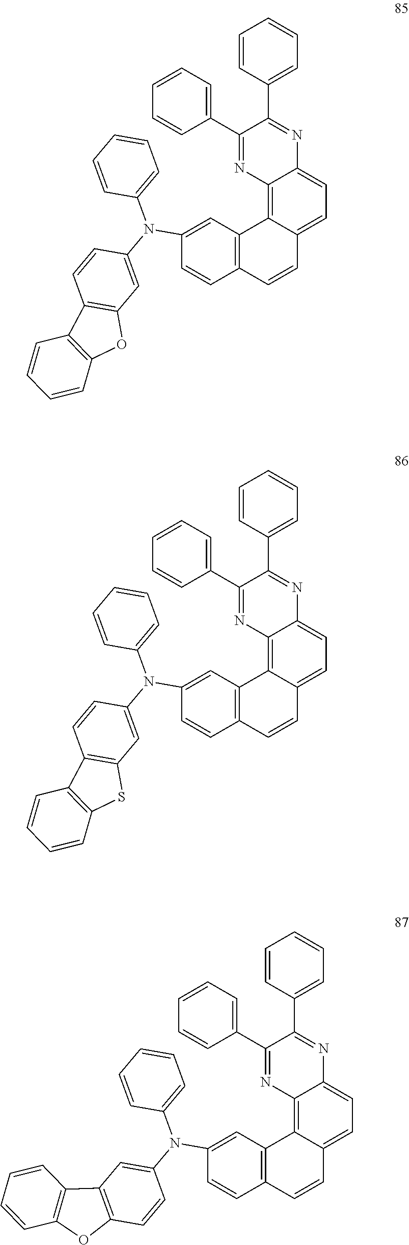 [SMILES: c1ccc(-c2nc3ccc4ccc5ccc(N(c6ccccc6)c6ccc7c(c6)oc6ccccc67)cc5c4c3nc2-c2ccccc2)cc1 ; c1ccc(-c2nc3ccc4ccc5ccc(N(c6ccccc6)c6ccc7c(c6)sc6ccccc67)cc5c4c3nc2-c2ccccc2)cc1 ; c1ccc(-c2nc3ccc4ccc5ccc(N(c6ccccc6)c6ccc7oc8ccccc8c7c6)cc5c4c3nc2-c2ccccc2)cc1]

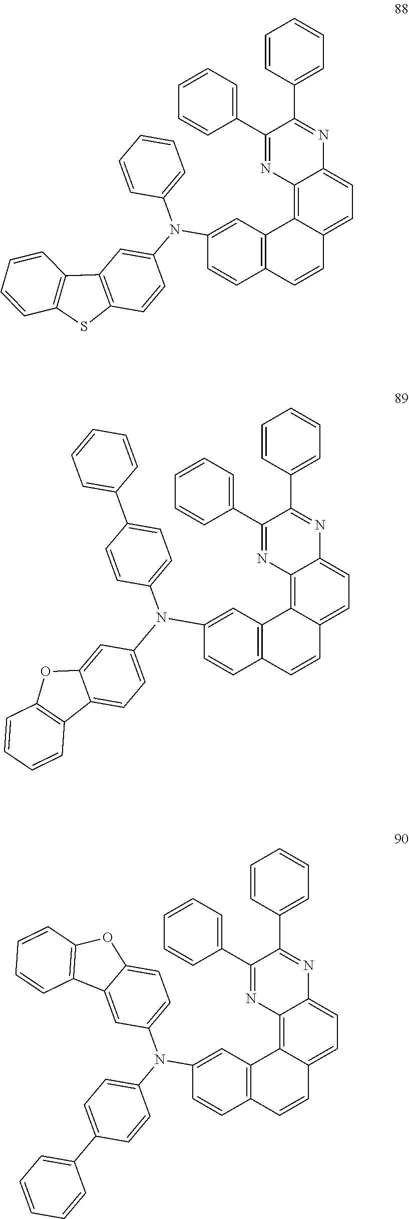 [SMILES: c1ccc(-c2nc3ccc4ccc5ccc(N(c6ccccc6)c6ccc7sc8ccccc8c7c6)cc5c4c3nc2-c2ccccc2)cc1 ; c1ccc(-c2ccc(N(c3ccc4c(c3)oc3ccccc34)c3ccc4ccc5ccc6nc(-c7ccccc7)c(-c7ccccc7)nc6c5c4c3)cc2)cc1 ; c1ccc(-c2ccc(N(c3ccc4oc5ccccc5c4c3)c3ccc4ccc5ccc6nc(-c7ccccc7)c(-c7ccccc7)nc6c5c4c3)cc2)cc1]

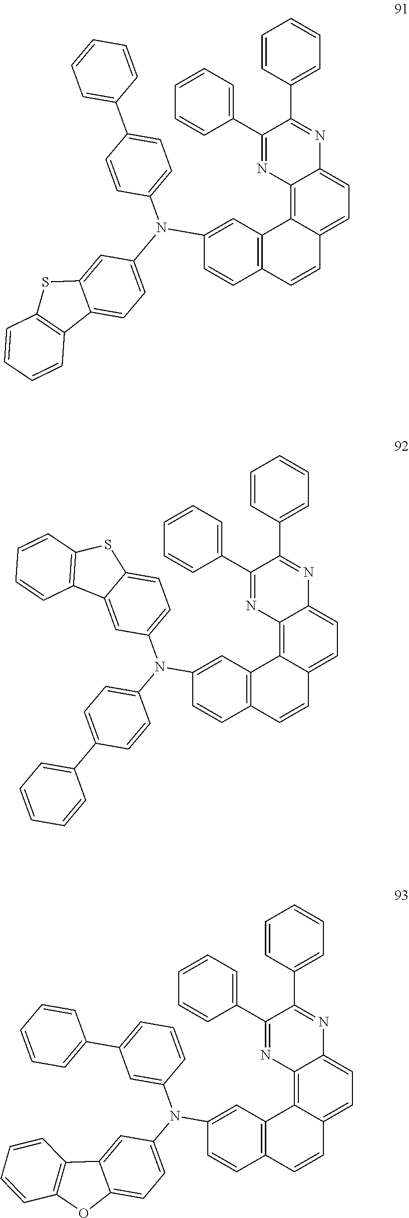 [SMILES: c1ccc(-c2ccc(N(c3ccc4c(c3)sc3ccccc34)c3ccc4ccc5ccc6nc(-c7ccccc7)c(-c7ccccc7)nc6c5c4c3)cc2)cc1 ; c1ccc(-c2ccc(N(c3ccc4sc5ccccc5c4c3)c3ccc4ccc5ccc6nc(-c7ccccc7)c(-c7ccccc7)nc6c5c4c3)cc2)cc1 ; c1ccc(-c2cccc(N(c3ccc4oc5ccccc5c4c3)c3ccc4ccc5ccc6nc(-c7ccccc7)c(-c7ccccc7)nc6c5c4c3)c2)cc1]

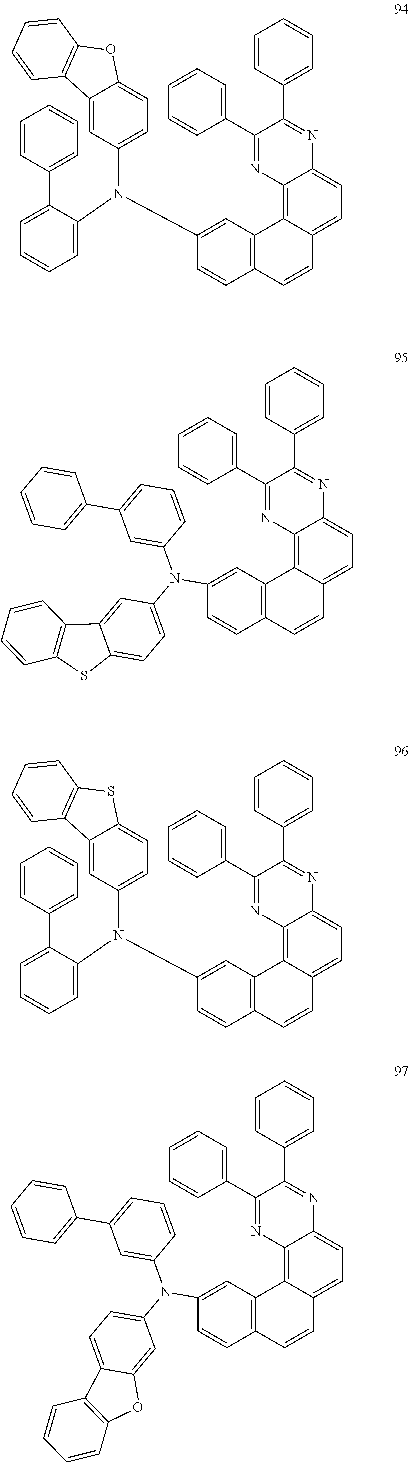 [SMILES: c1ccc(-c2ccccc2N(c2ccc3oc4ccccc4c3c2)c2ccc3ccc4ccc5nc(-c6ccccc6)c(-c6ccccc6)nc5c4c3c2)cc1 ; c1ccc(-c2cccc(N(c3ccc4sc5ccccc5c4c3)c3ccc4ccc5ccc6nc(-c7ccccc7)c(-c7ccccc7)nc6c5c4c3)c2)cc1 ; c1ccc(-c2ccccc2N(c2ccc3sc4ccccc4c3c2)c2ccc3ccc4ccc5nc(-c6ccccc6)c(-c6ccccc6)nc5c4c3c2)cc1 ; c1ccc(-c2cccc(N(c3ccc4c(c3)oc3ccccc34)c3ccc4ccc5ccc6nc(-c7ccccc7)c(-c7ccccc7)nc6c5c4c3)c2)cc1]

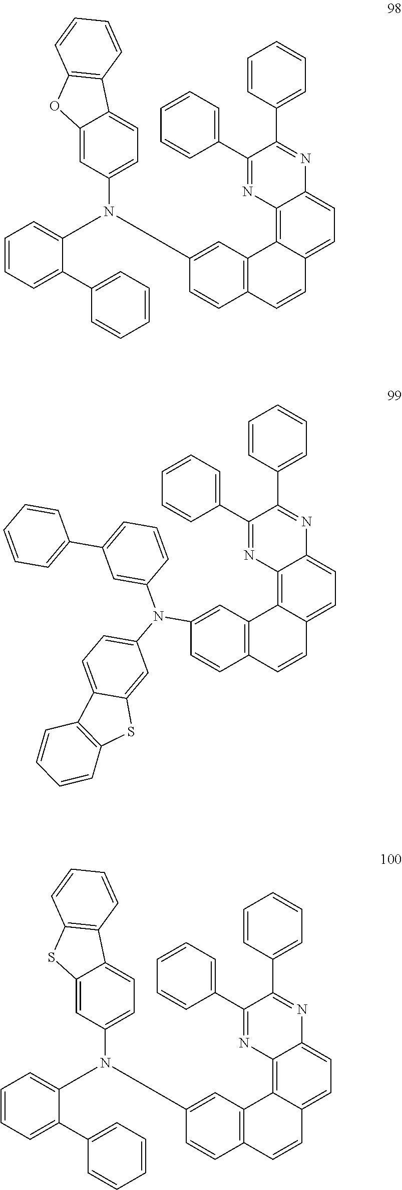 [SMILES: c1ccc(-c2ccccc2N(c2ccc3c(c2)oc2ccccc23)c2ccc3ccc4ccc5nc(-c6ccccc6)c(-c6ccccc6)nc5c4c3c2)cc1 ; c1ccc(-c2cccc(N(c3ccc4c(c3)sc3ccccc34)c3ccc4ccc5ccc6nc(-c7ccccc7)c(-c7ccccc7)nc6c5c4c3)c2)cc1 ; c1ccc(-c2ccccc2N(c2ccc3c(c2)sc2ccccc23)c2ccc3ccc4ccc5nc(-c6ccccc6)c(-c6ccccc6)nc5c4c3c2)cc1]

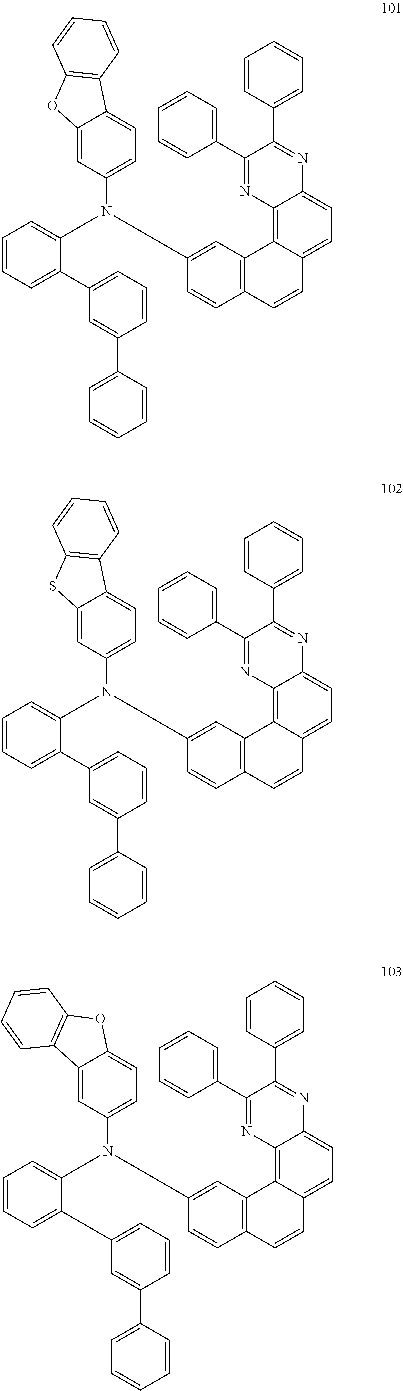 [SMILES: c1ccc(-c2cccc(-c3ccccc3N(c3ccc4c(c3)oc3ccccc34)c3ccc4ccc5ccc6nc(-c7ccccc7)c(-c7ccccc7)nc6c5c4c3)c2)cc1 ; c1ccc(-c2cccc(-c3ccccc3N(c3ccc4c(c3)sc3ccccc34)c3ccc4ccc5ccc6nc(-c7ccccc7)c(-c7ccccc7)nc6c5c4c3)c2)cc1 ; c1ccc(-c2cccc(-c3ccccc3N(c3ccc4oc5ccccc5c4c3)c3ccc4ccc5ccc6nc(-c7ccccc7)c(-c7ccccc7)nc6c5c4c3)c2)cc1]

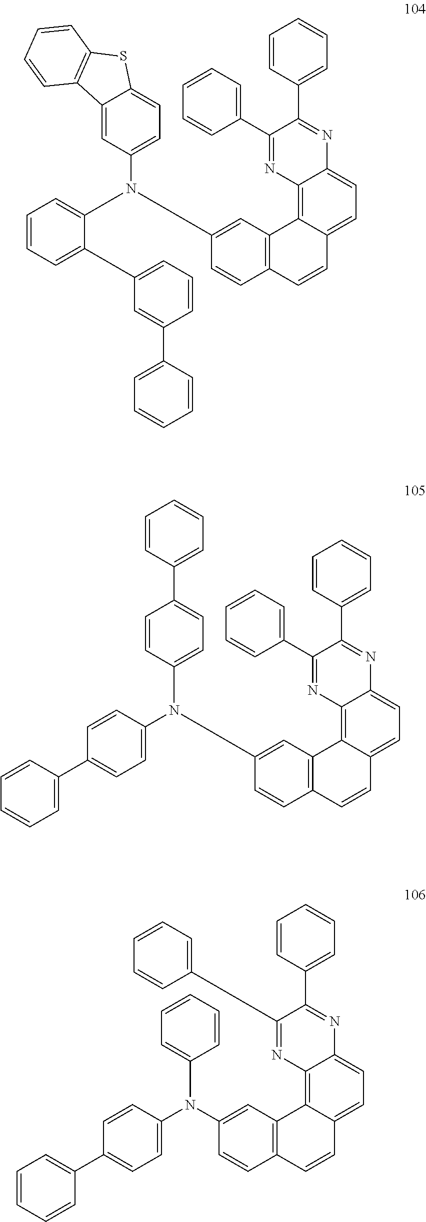 [SMILES: c1ccc(-c2cccc(-c3ccccc3N(c3ccc4sc5ccccc5c4c3)c3ccc4ccc5ccc6nc(-c7ccccc7)c(-c7ccccc7)nc6c5c4c3)c2)cc1 ; c1ccc(-c2ccc(N(c3ccc(-c4ccccc4)cc3)c3ccc4ccc5ccc6nc(-c7ccccc7)c(-c7ccccc7)nc6c5c4c3)cc2)cc1 ; c1ccc(-c2ccc(N(c3ccccc3)c3ccc4ccc5ccc6nc(-c7ccccc7)c(-c7ccccc7)nc6c5c4c3)cc2)cc1]

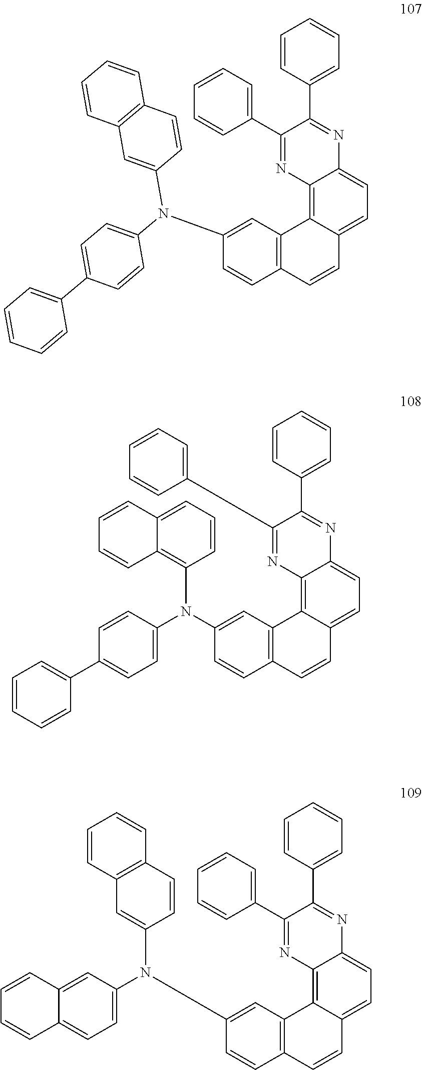 [SMILES: c1ccc(-c2ccc(N(c3ccc4ccccc4c3)c3ccc4ccc5ccc6nc(-c7ccccc7)c(-c7ccccc7)nc6c5c4c3)cc2)cc1 ; c1ccc(-c2ccc(N(c3ccc4ccc5ccc6nc(-c7ccccc7)c(-c7ccccc7)nc6c5c4c3)c3cccc4ccccc34)cc2)cc1 ; c1ccc(-c2nc3ccc4ccc5ccc(N(c6ccc7ccccc7c6)c6ccc7ccccc7c6)cc5c4c3nc2-c2ccccc2)cc1]

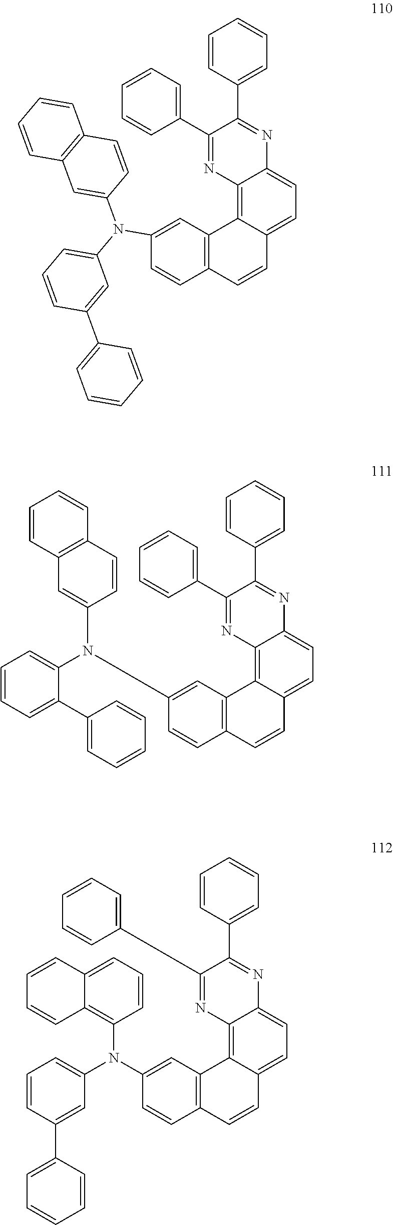 [SMILES: c1ccc(-c2cccc(N(c3ccc4ccccc4c3)c3ccc4ccc5ccc6nc(-c7ccccc7)c(-c7ccccc7)nc6c5c4c3)c2)cc1 ; c1ccc(-c2ccccc2N(c2ccc3ccccc3c2)c2ccc3ccc4ccc5nc(-c6ccccc6)c(-c6ccccc6)nc5c4c3c2)cc1 ; c1ccc(-c2cccc(N(c3ccc4ccc5ccc6nc(-c7ccccc7)c(-c7ccccc7)nc6c5c4c3)c3cccc4ccccc34)c2)cc1]

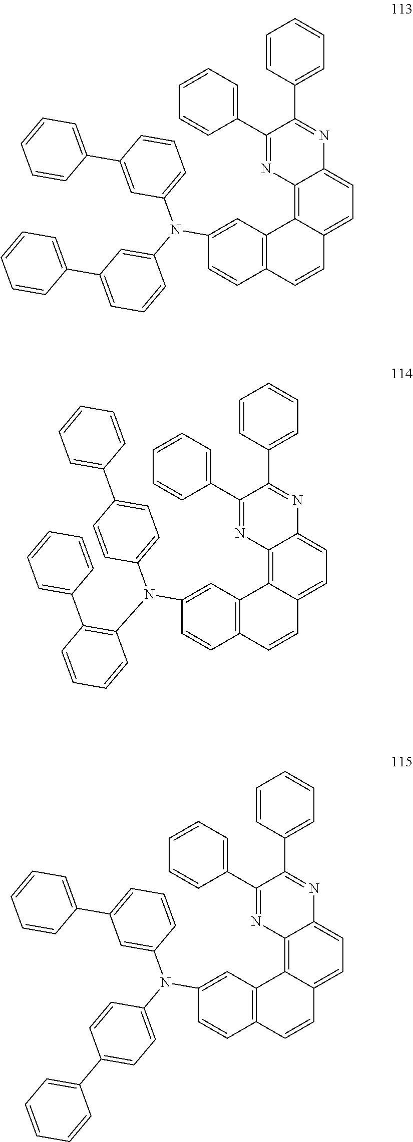 [SMILES: c1ccc(-c2cccc(N(c3cccc(-c4ccccc4)c3)c3ccc4ccc5ccc6nc(-c7ccccc7)c(-c7ccccc7)nc6c5c4c3)c2)cc1 ; c1ccc(-c2ccc(N(c3ccc4ccc5ccc6nc(-c7ccccc7)c(-c7ccccc7)nc6c5c4c3)c3ccccc3-c3ccccc3)cc2)cc1 ; c1ccc(-c2ccc(N(c3cccc(-c4ccccc4)c3)c3ccc4ccc5ccc6nc(-c7ccccc7)c(-c7ccccc7)nc6c5c4c3)cc2)cc1]

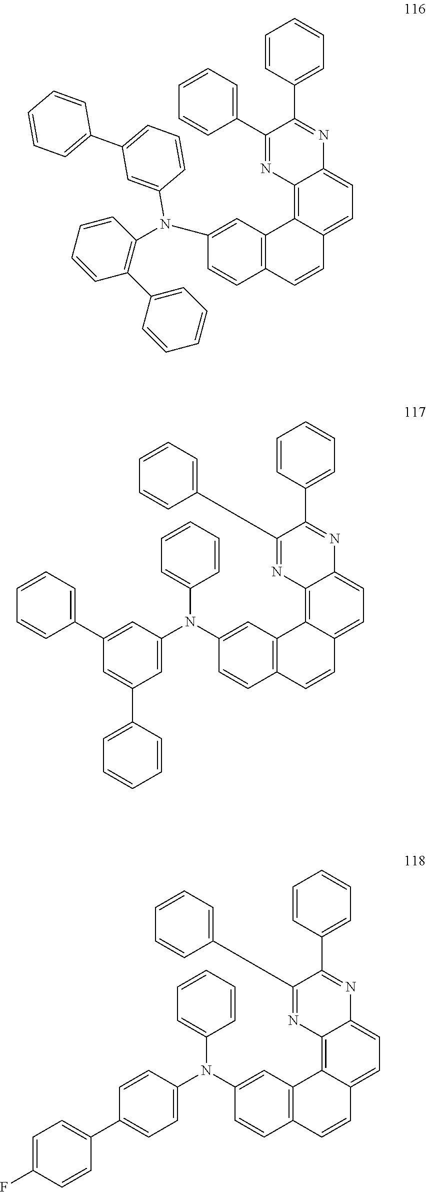 [SMILES: c1ccc(-c2cccc(N(c3ccc4ccc5ccc6nc(-c7ccccc7)c(-c7ccccc7)nc6c5c4c3)c3ccccc3-c3ccccc3)c2)cc1 ; c1ccc(-c2cc(-c3ccccc3)cc(N(c3ccccc3)c3ccc4ccc5ccc6nc(-c7ccccc7)c(-c7ccccc7)nc6c5c4c3)c2)cc1 ; Fc1ccc(-c2ccc(N(c3ccccc3)c3ccc4ccc5ccc6nc(-c7ccccc7)c(-c7ccccc7)nc6c5c4c3)cc2)cc1]

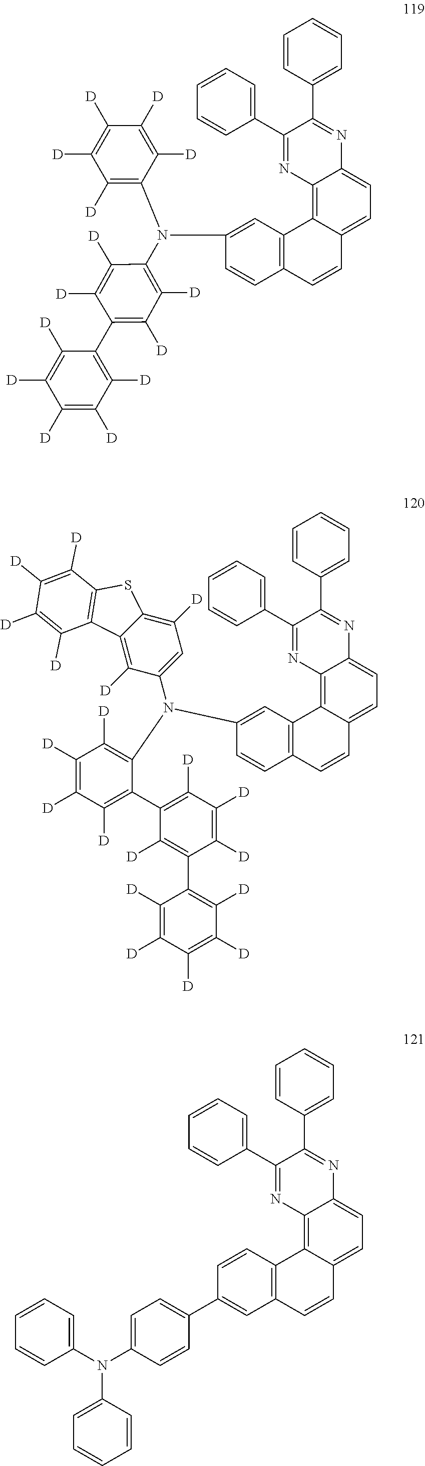 [SMILES: [2H]c1c([2H])c([2H])c(-c2c([2H])c([2H])c(N(c3ccc4ccc5ccc6nc(-c7ccccc7)c(-c7ccccc7)nc6c5c4c3)c3c([2H])c([2H])c([2H])c([2H])c3[2H])c([2H])c2[2H])c([2H])c1[2H] ; [2H]c1c([2H])c([2H])c(-c2c([2H])c([2H])c([2H])c(-c3c([2H])c([2H])c([2H])c([2H])c3N(c3ccc4ccc5ccc6nc(-c7ccccc7)c(-c7ccccc7)nc6c5c4c3)c3cc([2H])c4sc5c([2H])c([2H])c([2H])c([2H])c5c4c3[2H])c2[2H])c([2H])c1[2H] ; c1ccc(-c2nc3ccc4ccc5cc(-c6ccc(N(c7ccccc7)c7ccccc7)cc6)ccc5c4c3nc2-c2ccccc2)cc1]

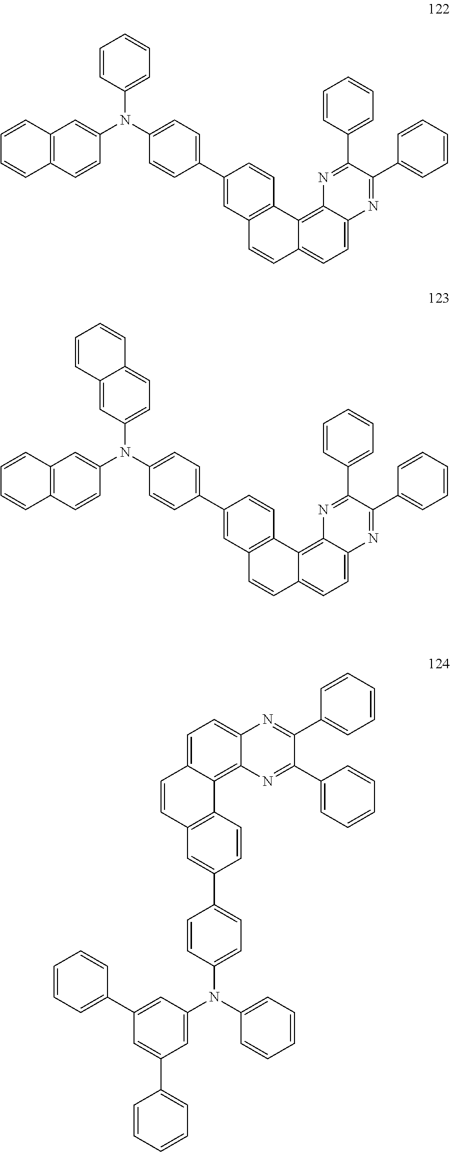 [SMILES: c1ccc(-c2nc3ccc4ccc5cc(-c6ccc(N(c7ccccc7)c7ccc8ccccc8c7)cc6)ccc5c4c3nc2-c2ccccc2)cc1 ; c1ccc(-c2nc3ccc4ccc5cc(-c6ccc(N(c7ccc8ccccc8c7)c7ccc8ccccc8c7)cc6)ccc5c4c3nc2-c2ccccc2)cc1 ; c1ccc(-c2cc(-c3ccccc3)cc(N(c3ccccc3)c3ccc(-c4ccc5c(ccc6ccc7nc(-c8ccccc8)c(-c8ccccc8)nc7c65)c4)cc3)c2)cc1]

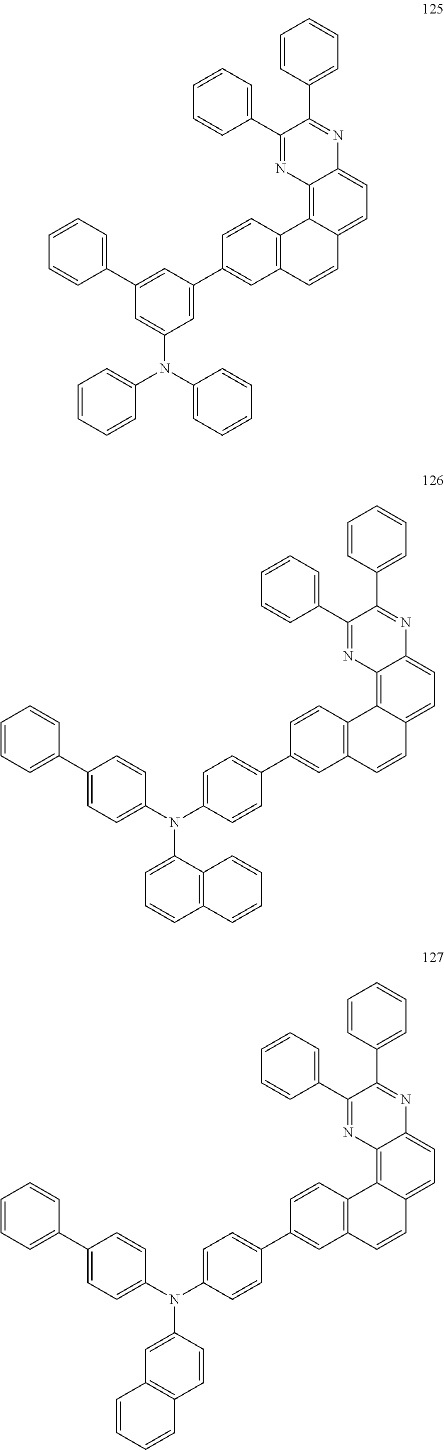 [SMILES: c1ccc(-c2cc(-c3ccc4c(ccc5ccc6nc(-c7ccccc7)c(-c7ccccc7)nc6c54)c3)cc(N(c3ccccc3)c3ccccc3)c2)cc1 ; c1ccc(-c2ccc(N(c3ccc(-c4ccc5c(ccc6ccc7nc(-c8ccccc8)c(-c8ccccc8)nc7c65)c4)cc3)c3cccc4ccccc34)cc2)cc1 ; c1ccc(-c2ccc(N(c3ccc(-c4ccc5c(ccc6ccc7nc(-c8ccccc8)c(-c8ccccc8)nc7c65)c4)cc3)c3ccc4ccccc4c3)cc2)cc1]

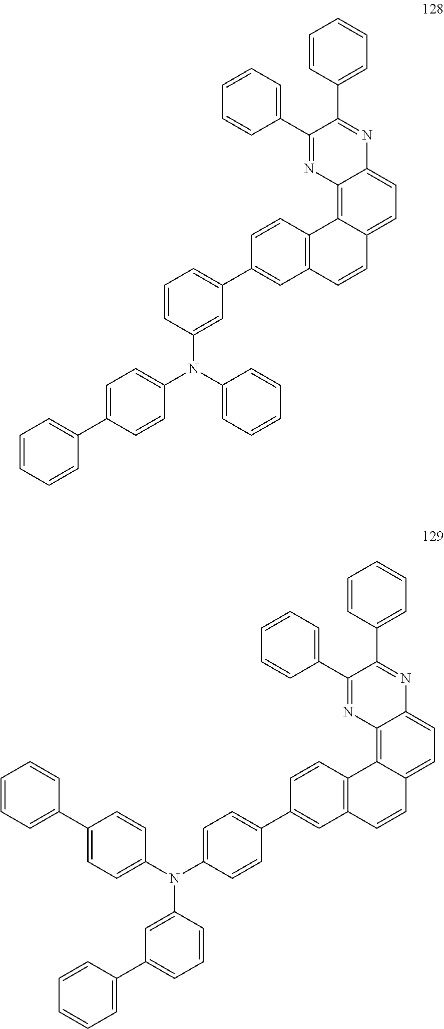 [SMILES: c1ccc(-c2ccc(N(c3ccccc3)c3cccc(-c4ccc5c(ccc6ccc7nc(-c8ccccc8)c(-c8ccccc8)nc7c65)c4)c3)cc2)cc1 ; c1ccc(-c2ccc(N(c3ccc(-c4ccc5c(ccc6ccc7nc(-c8ccccc8)c(-c8ccccc8)nc7c65)c4)cc3)c3cccc(-c4ccccc4)c3)cc2)cc1]

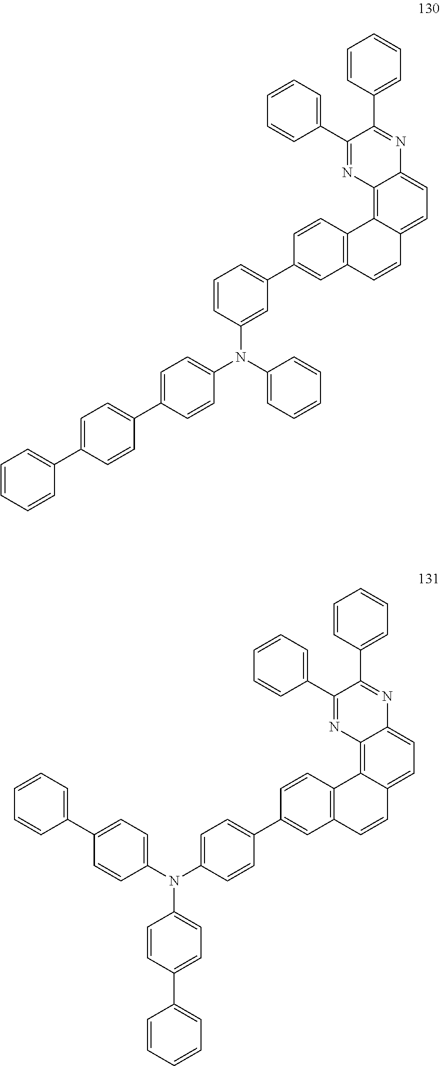 [SMILES: c1ccc(-c2ccc(-c3ccc(N(c4ccccc4)c4cccc(-c5ccc6c(ccc7ccc8nc(-c9ccccc9)c(-c9ccccc9)nc8c76)c5)c4)cc3)cc2)cc1 ; c1ccc(-c2ccc(N(c3ccc(-c4ccccc4)cc3)c3ccc(-c4ccc5c(ccc6ccc7nc(-c8ccccc8)c(-c8ccccc8)nc7c65)c4)cc3)cc2)cc1]

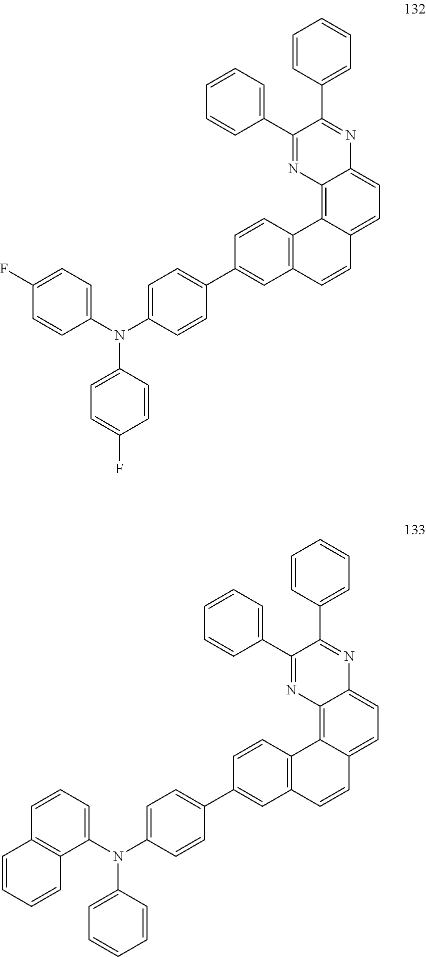 [SMILES: Fc1ccc(N(c2ccc(F)cc2)c2ccc(-c3ccc4c(ccc5ccc6nc(-c7ccccc7)c(-c7ccccc7)nc6c54)c3)cc2)cc1 ; c1ccc(-c2nc3ccc4ccc5cc(-c6ccc(N(c7ccccc7)c7cccc8ccccc78)cc6)ccc5c4c3nc2-c2ccccc2)cc1]

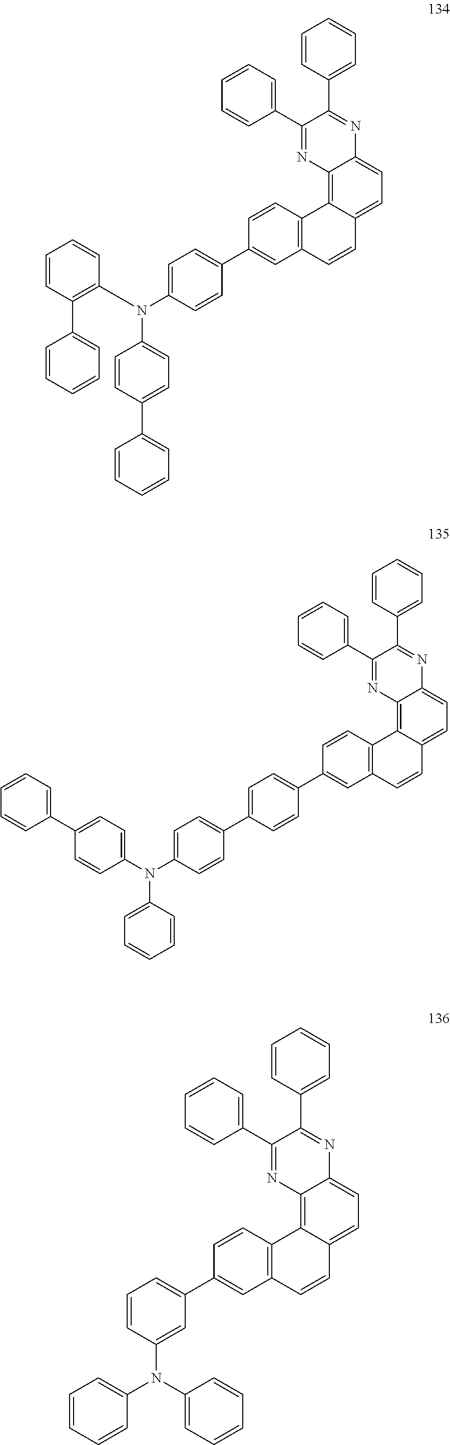 [SMILES: c1ccc(-c2ccc(N(c3ccc(-c4ccc5c(ccc6ccc7nc(-c8ccccc8)c(-c8ccccc8)nc7c65)c4)cc3)c3ccccc3-c3ccccc3)cc2)cc1 ; c1ccc(-c2ccc(N(c3ccccc3)c3ccc(-c4ccc(-c5ccc6c(ccc7ccc8nc(-c9ccccc9)c(-c9ccccc9)nc8c76)c5)cc4)cc3)cc2)cc1 ; c1ccc(-c2nc3ccc4ccc5cc(-c6cccc(N(c7ccccc7)c7ccccc7)c6)ccc5c4c3nc2-c2ccccc2)cc1]

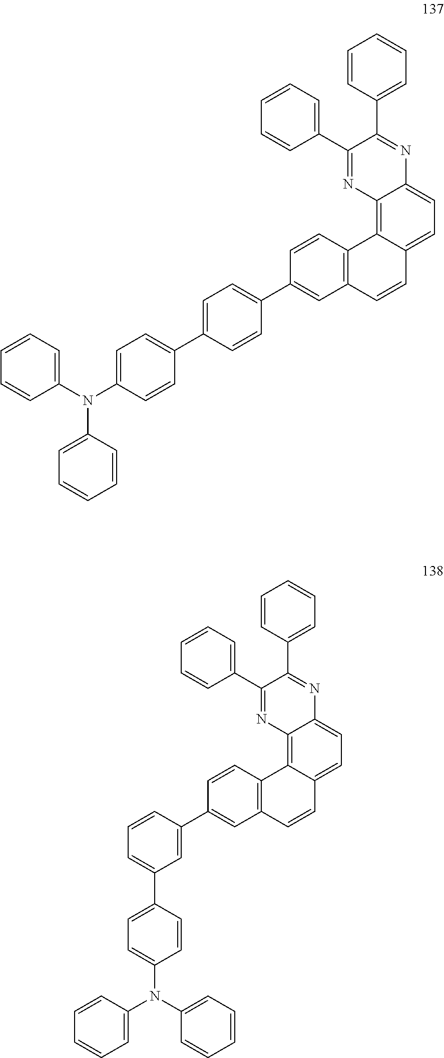 [SMILES: c1ccc(-c2nc3ccc4ccc5cc(-c6ccc(-c7ccc(N(c8ccccc8)c8ccccc8)cc7)cc6)ccc5c4c3nc2-c2ccccc2)cc1 ; c1ccc(-c2nc3ccc4ccc5cc(-c6cccc(-c7ccc(N(c8ccccc8)c8ccccc8)cc7)c6)ccc5c4c3nc2-c2ccccc2)cc1]

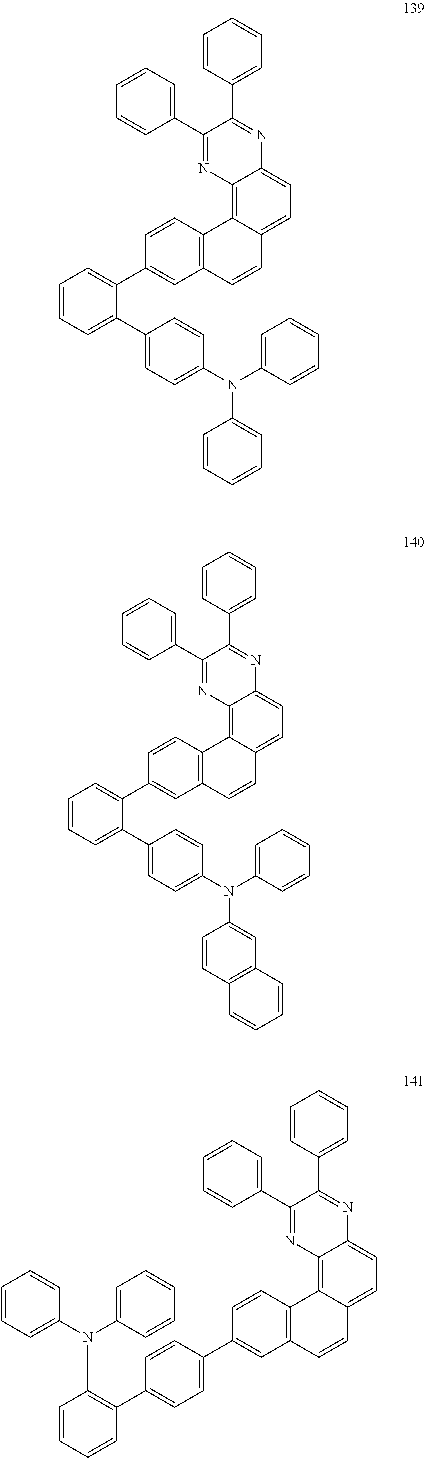 [SMILES: c1ccc(-c2nc3ccc4ccc5cc(-c6ccccc6-c6ccc(N(c7ccccc7)c7ccccc7)cc6)ccc5c4c3nc2-c2ccccc2)cc1 ; c1ccc(-c2nc3ccc4ccc5cc(-c6ccccc6-c6ccc(N(c7ccccc7)c7ccc8ccccc8c7)cc6)ccc5c4c3nc2-c2ccccc2)cc1 ; c1ccc(-c2nc3ccc4ccc5cc(-c6ccc(-c7ccccc7N(c7ccccc7)c7ccccc7)cc6)ccc5c4c3nc2-c2ccccc2)cc1]

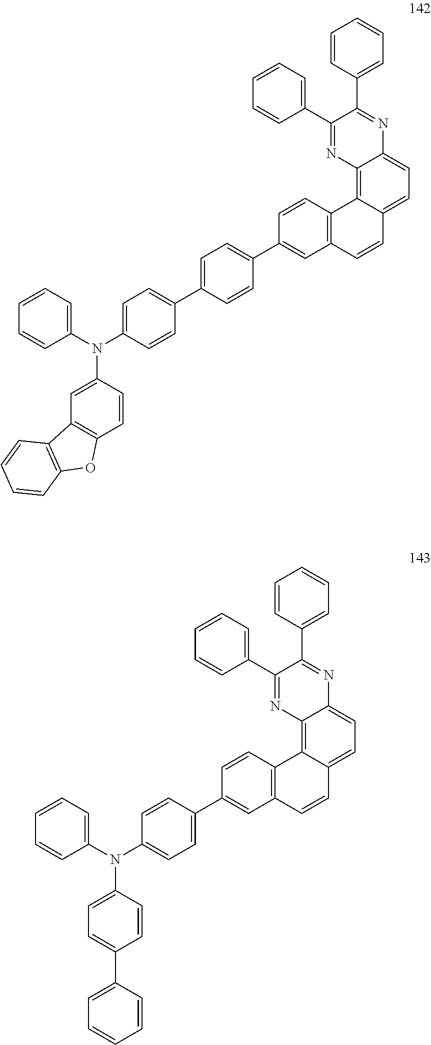 [SMILES: c1ccc(-c2nc3ccc4ccc5cc(-c6ccc(-c7ccc(N(c8ccccc8)c8ccc9oc%10ccccc%10c9c8)cc7)cc6)ccc5c4c3nc2-c2ccccc2)cc1 ; c1ccc(-c2ccc(N(c3ccccc3)c3ccc(-c4ccc5c(ccc6ccc7nc(-c8ccccc8)c(-c8ccccc8)nc7c65)c4)cc3)cc2)cc1]

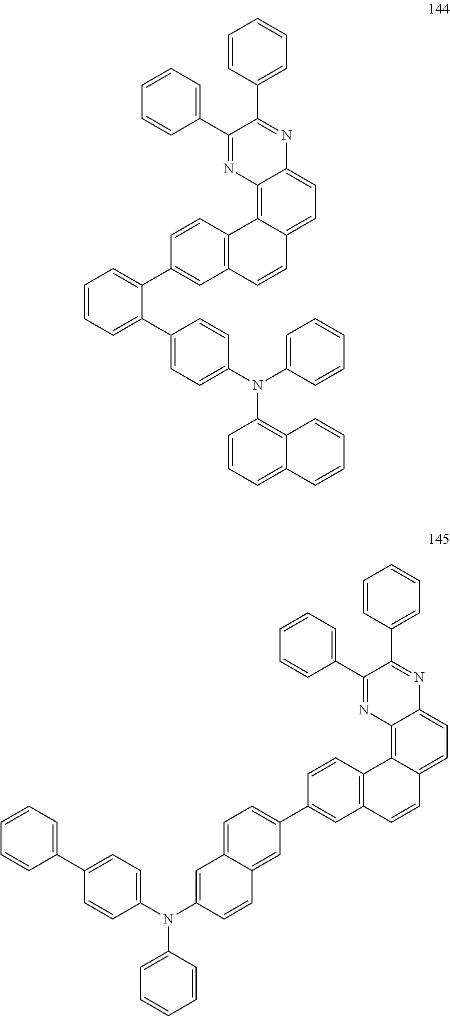 [SMILES: c1ccc(-c2nc3ccc4ccc5cc(-c6ccccc6-c6ccc(N(c7ccccc7)c7cccc8ccccc78)cc6)ccc5c4c3nc2-c2ccccc2)cc1 ; c1ccc(-c2ccc(N(c3ccccc3)c3ccc4cc(-c5ccc6c(ccc7ccc8nc(-c9ccccc9)c(-c9ccccc9)nc8c76)c5)ccc4c3)cc2)cc1]

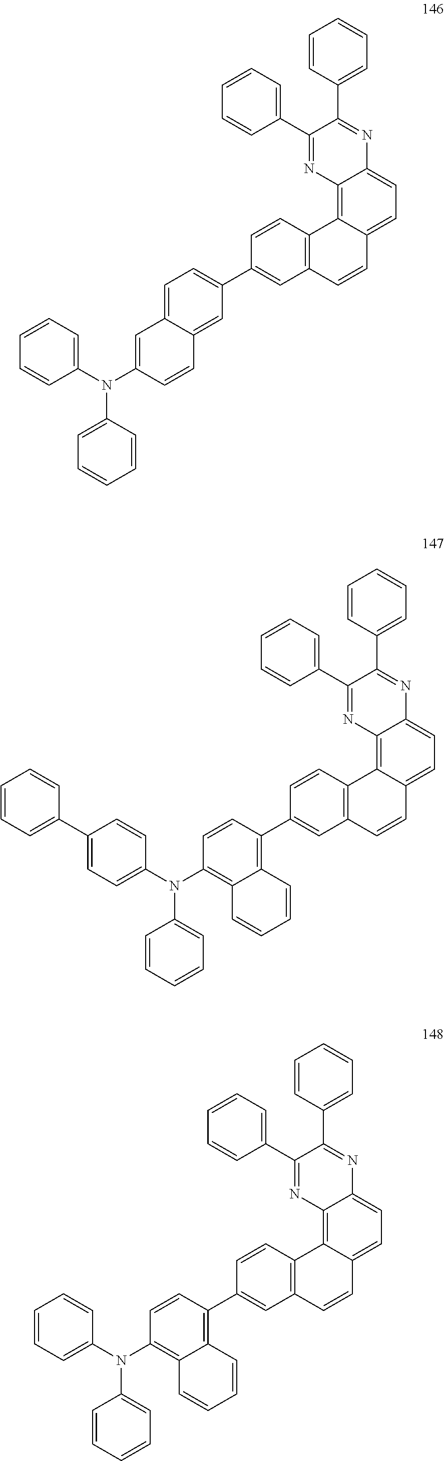 [SMILES: c1ccc(-c2nc3ccc4ccc5cc(-c6ccc7cc(N(c8ccccc8)c8ccccc8)ccc7c6)ccc5c4c3nc2-c2ccccc2)cc1 ; c1ccc(-c2ccc(N(c3ccccc3)c3ccc(-c4ccc5c(ccc6ccc7nc(-c8ccccc8)c(-c8ccccc8)nc7c65)c4)c4ccccc34)cc2)cc1 ; c1ccc(-c2nc3ccc4ccc5cc(-c6ccc(N(c7ccccc7)c7ccccc7)c7ccccc67)ccc5c4c3nc2-c2ccccc2)cc1]

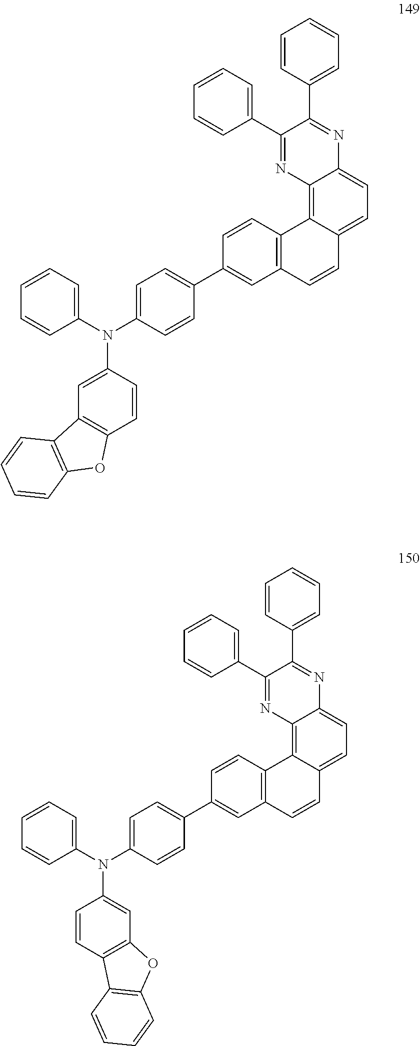 [SMILES: c1ccc(-c2nc3ccc4ccc5cc(-c6ccc(N(c7ccccc7)c7ccc8oc9ccccc9c8c7)cc6)ccc5c4c3nc2-c2ccccc2)cc1 ; c1ccc(-c2nc3ccc4ccc5cc(-c6ccc(N(c7ccccc7)c7ccc8c(c7)oc7ccccc78)cc6)ccc5c4c3nc2-c2ccccc2)cc1]

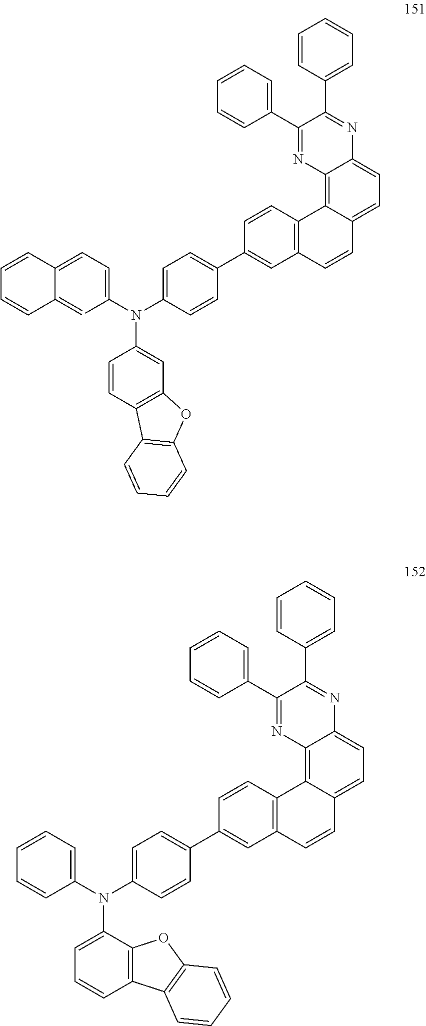

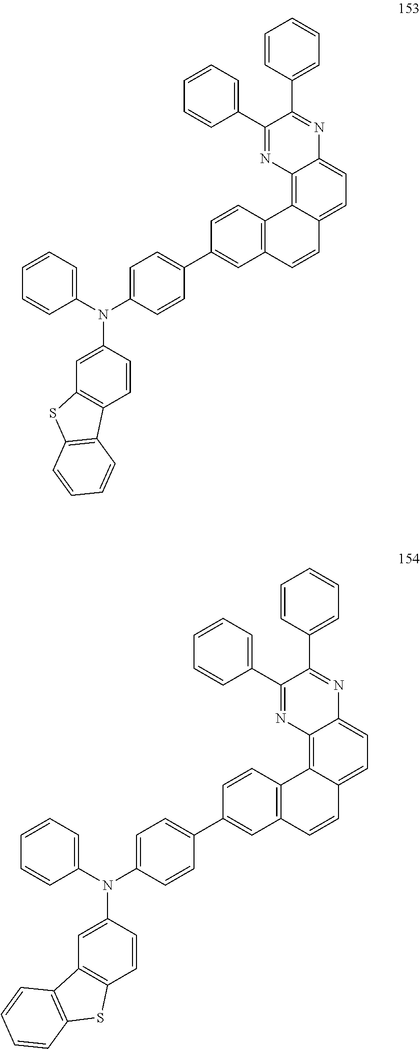 [SMILES: c1ccc(-c2nc3ccc4ccc5cc(-c6ccc(N(c7ccccc7)c7ccc8c(c7)sc7ccccc78)cc6)ccc5c4c3nc2-c2ccccc2)cc1 ; c1ccc(-c2nc3ccc4ccc5cc(-c6ccc(N(c7ccccc7)c7ccc8sc9ccccc9c8c7)cc6)ccc5c4c3nc2-c2ccccc2)cc1]

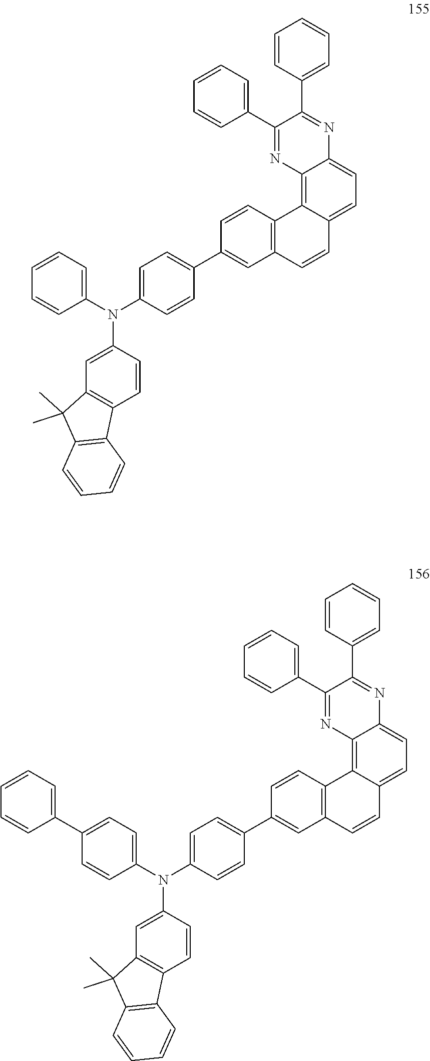 [SMILES: CC1(C)c2ccccc2-c2ccc(N(c3ccccc3)c3ccc(-c4ccc5c(ccc6ccc7nc(-c8ccccc8)c(-c8ccccc8)nc7c65)c4)cc3)cc21 ; CC1(C)c2ccccc2-c2ccc(N(c3ccc(-c4ccccc4)cc3)c3ccc(-c4ccc5c(ccc6ccc7nc(-c8ccccc8)c(-c8ccccc8)nc7c65)c4)cc3)cc21]

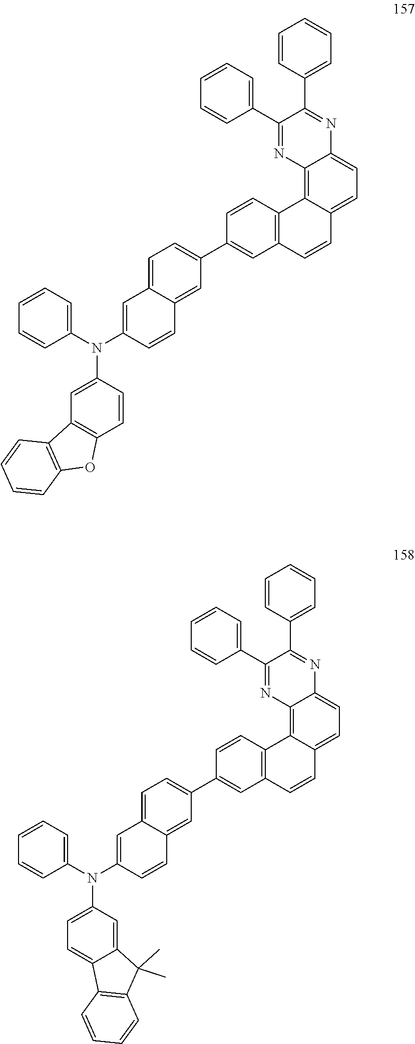 [SMILES: c1ccc(-c2nc3ccc4ccc5cc(-c6ccc7cc(N(c8ccccc8)c8ccc9oc%10ccccc%10c9c8)ccc7c6)ccc5c4c3nc2-c2ccccc2)cc1 ; CC1(C)c2ccccc2-c2ccc(N(c3ccccc3)c3ccc4cc(-c5ccc6c(ccc7ccc8nc(-c9ccccc9)c(-c9ccccc9)nc8c76)c5)ccc4c3)cc21]

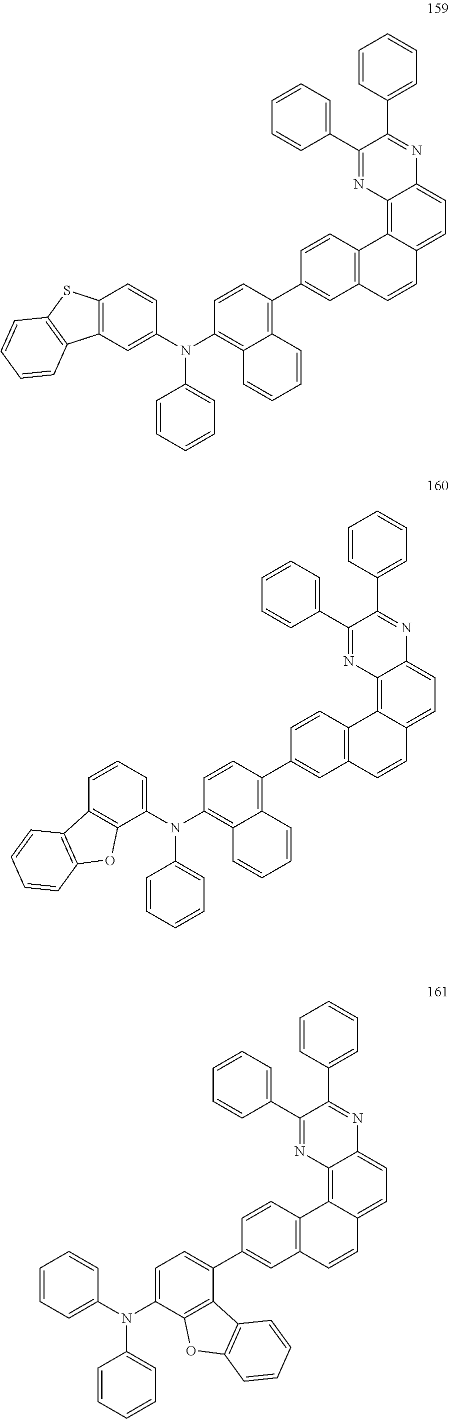 [SMILES: c1ccc(-c2nc3ccc4ccc5cc(-c6ccc(N(c7ccccc7)c7ccc8sc9ccccc9c8c7)c7ccccc67)ccc5c4c3nc2-c2ccccc2)cc1 ; c1ccc(-c2nc3ccc4ccc5cc(-c6ccc(N(c7ccccc7)c7cccc8c7oc7ccccc78)c7ccccc67)ccc5c4c3nc2-c2ccccc2)cc1 ; c1ccc(-c2nc3ccc4ccc5cc(-c6ccc(N(c7ccccc7)c7ccccc7)c7oc8ccccc8c67)ccc5c4c3nc2-c2ccccc2)cc1]

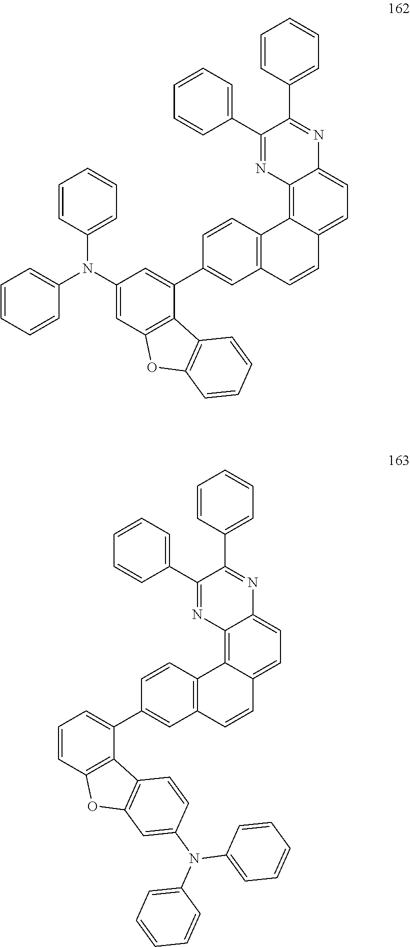 [SMILES: c1ccc(-c2nc3ccc4ccc5cc(-c6cc(N(c7ccccc7)c7ccccc7)cc7oc8ccccc8c67)ccc5c4c3nc2-c2ccccc2)cc1 ; c1ccc(-c2nc3ccc4ccc5cc(-c6cccc7oc8cc(N(c9ccccc9)c9ccccc9)ccc8c67)ccc5c4c3nc2-c2ccccc2)cc1]

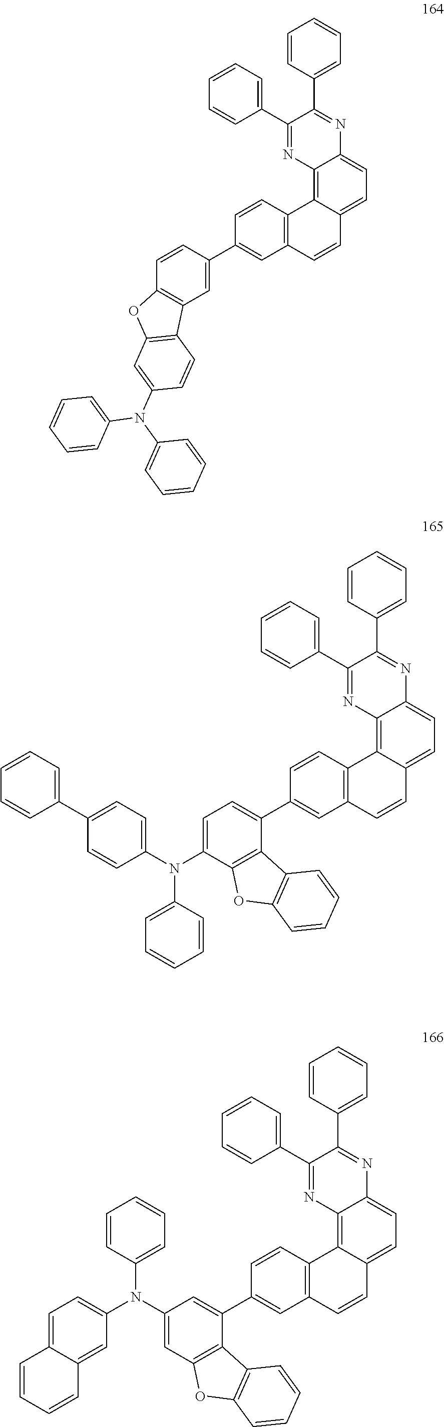 [SMILES: c1ccc(-c2nc3ccc4ccc5cc(-c6ccc7oc8cc(N(c9ccccc9)c9ccccc9)ccc8c7c6)ccc5c4c3nc2-c2ccccc2)cc1 ; c1ccc(-c2ccc(N(c3ccccc3)c3ccc(-c4ccc5c(ccc6ccc7nc(-c8ccccc8)c(-c8ccccc8)nc7c65)c4)c4c3oc3ccccc34)cc2)cc1 ; c1ccc(-c2nc3ccc4ccc5cc(-c6cc(N(c7ccccc7)c7ccc8ccccc8c7)cc7oc8ccccc8c67)ccc5c4c3nc2-c2ccccc2)cc1]

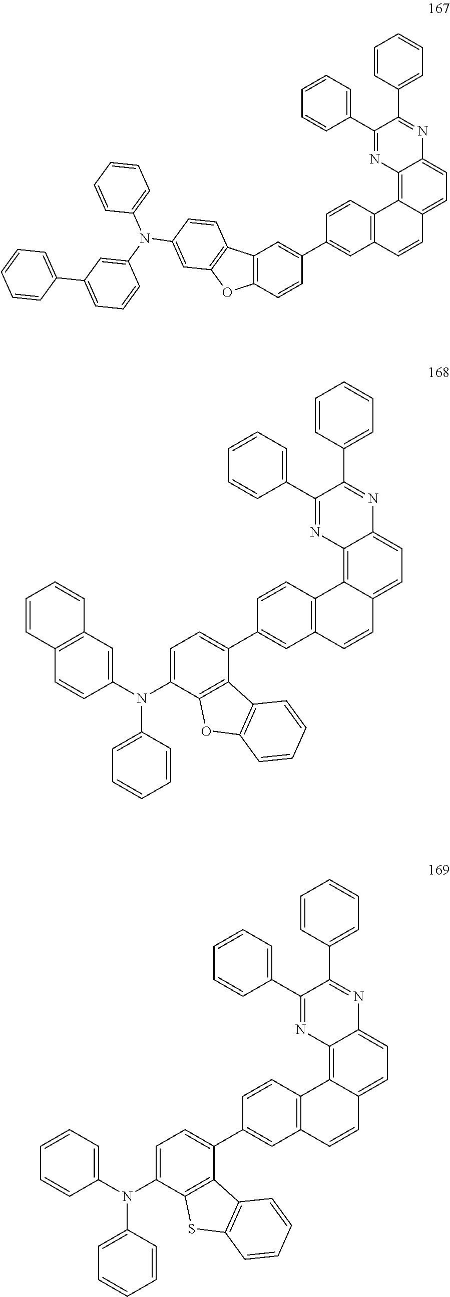 [SMILES: c1ccc(-c2cccc(N(c3ccccc3)c3ccc4c(c3)oc3ccc(-c5ccc6c(ccc7ccc8nc(-c9ccccc9)c(-c9ccccc9)nc8c76)c5)cc34)c2)cc1 ; c1ccc(-c2nc3ccc4ccc5cc(-c6ccc(N(c7ccccc7)c7ccc8ccccc8c7)c7oc8ccccc8c67)ccc5c4c3nc2-c2ccccc2)cc1 ; c1ccc(-c2nc3ccc4ccc5cc(-c6ccc(N(c7ccccc7)c7ccccc7)c7sc8ccccc8c67)ccc5c4c3nc2-c2ccccc2)cc1]

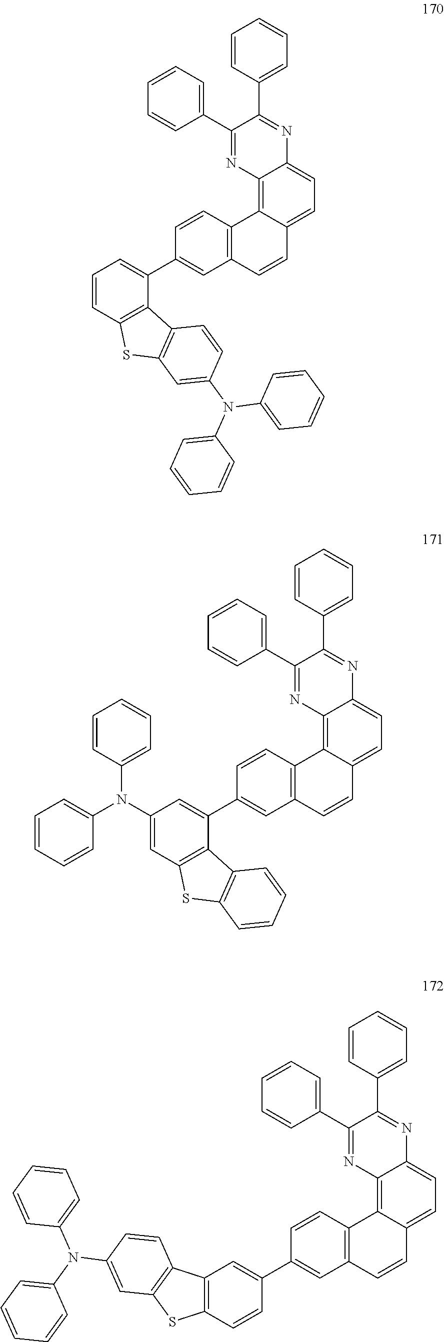 [SMILES: c1ccc(-c2nc3ccc4ccc5cc(-c6cccc7sc8cc(N(c9ccccc9)c9ccccc9)ccc8c67)ccc5c4c3nc2-c2ccccc2)cc1 ; c1ccc(-c2nc3ccc4ccc5cc(-c6cc(N(c7ccccc7)c7ccccc7)cc7sc8ccccc8c67)ccc5c4c3nc2-c2ccccc2)cc1 ; c1ccc(-c2nc3ccc4ccc5cc(-c6ccc7sc8cc(N(c9ccccc9)c9ccccc9)ccc8c7c6)ccc5c4c3nc2-c2ccccc2)cc1]

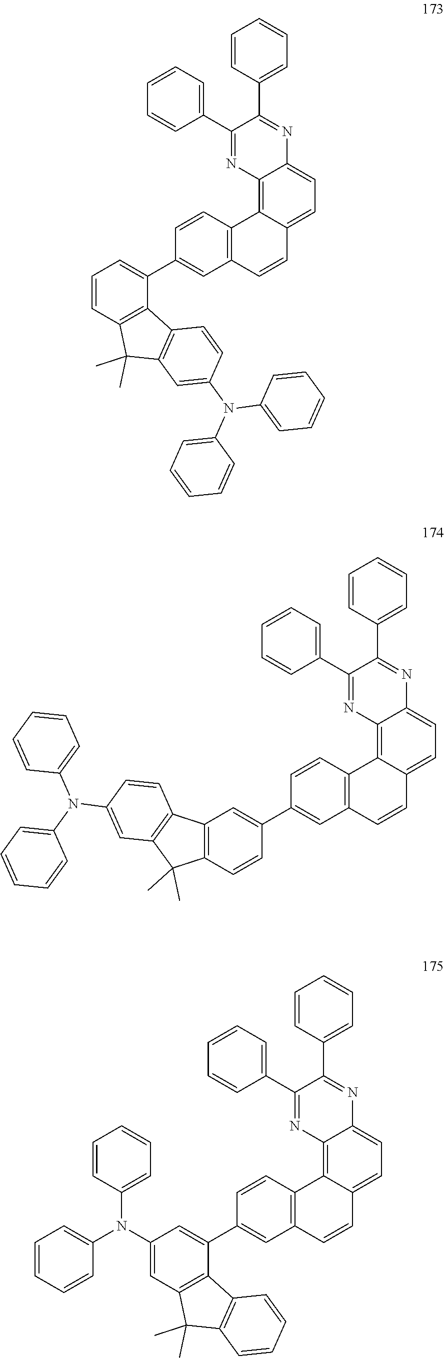 [SMILES: CC1(C)c2cc(N(c3ccccc3)c3ccccc3)ccc2-c2c(-c3ccc4c(ccc5ccc6nc(-c7ccccc7)c(-c7ccccc7)nc6c54)c3)cccc21 ; CC1(C)c2ccc(-c3ccc4c(ccc5ccc6nc(-c7ccccc7)c(-c7ccccc7)nc6c54)c3)cc2-c2ccc(N(c3ccccc3)c3ccccc3)cc21 ; CC1(C)c2ccccc2-c2c(-c3ccc4c(ccc5ccc6nc(-c7ccccc7)c(-c7ccccc7)nc6c54)c3)cc(N(c3ccccc3)c3ccccc3)cc21]

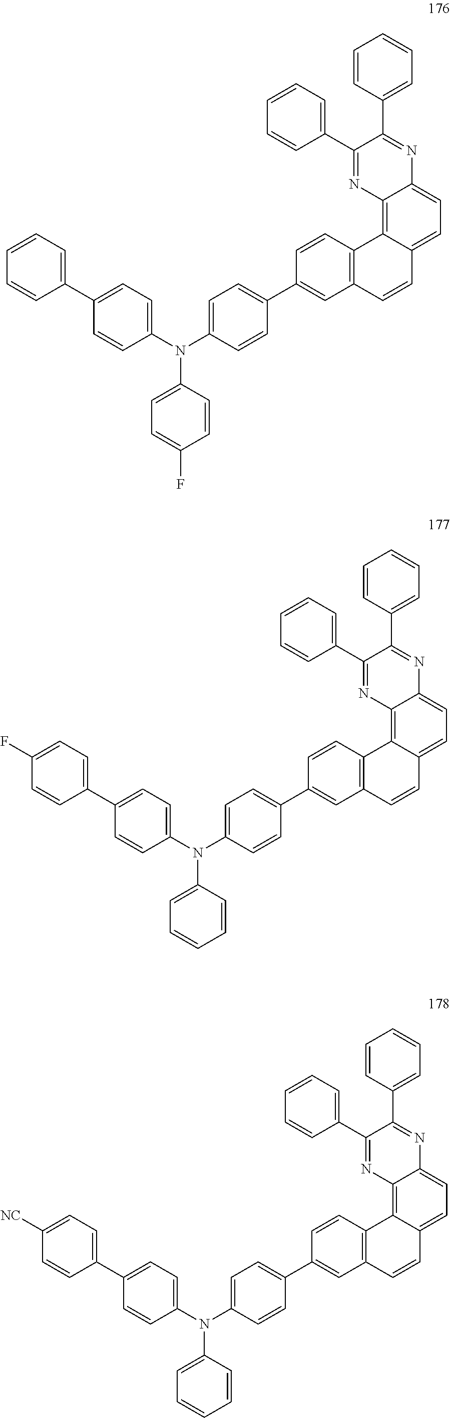 [SMILES: Fc1ccc(N(c2ccc(-c3ccccc3)cc2)c2ccc(-c3ccc4c(ccc5ccc6nc(-c7ccccc7)c(-c7ccccc7)nc6c54)c3)cc2)cc1 ; Fc1ccc(-c2ccc(N(c3ccccc3)c3ccc(-c4ccc5c(ccc6ccc7nc(-c8ccccc8)c(-c8ccccc8)nc7c65)c4)cc3)cc2)cc1 ; N#Cc1ccc(-c2ccc(N(c3ccccc3)c3ccc(-c4ccc5c(ccc6ccc7nc(-c8ccccc8)c(-c8ccccc8)nc7c65)c4)cc3)cc2)cc1]

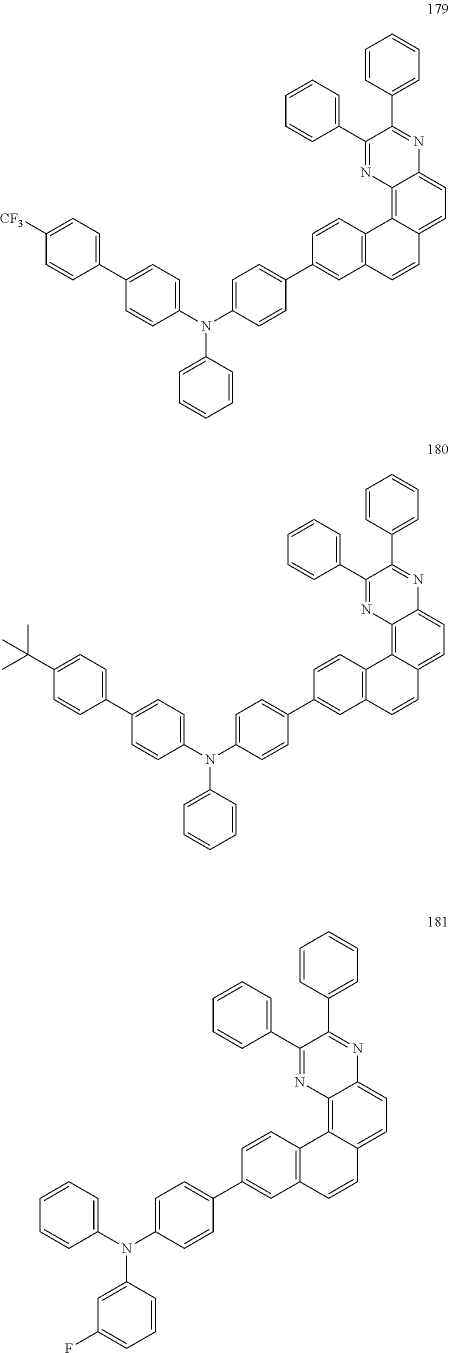 [SMILES: FC(F)(F)c1ccc(-c2ccc(N(c3ccccc3)c3ccc(-c4ccc5c(ccc6ccc7nc(-c8ccccc8)c(-c8ccccc8)nc7c65)c4)cc3)cc2)cc1 ; CC(C)(C)c1ccc(-c2ccc(N(c3ccccc3)c3ccc(-c4ccc5c(ccc6ccc7nc(-c8ccccc8)c(-c8ccccc8)nc7c65)c4)cc3)cc2)cc1 ; Fc1cccc(N(c2ccccc2)c2ccc(-c3ccc4c(ccc5ccc6nc(-c7ccccc7)c(-c7ccccc7)nc6c54)c3)cc2)c1]

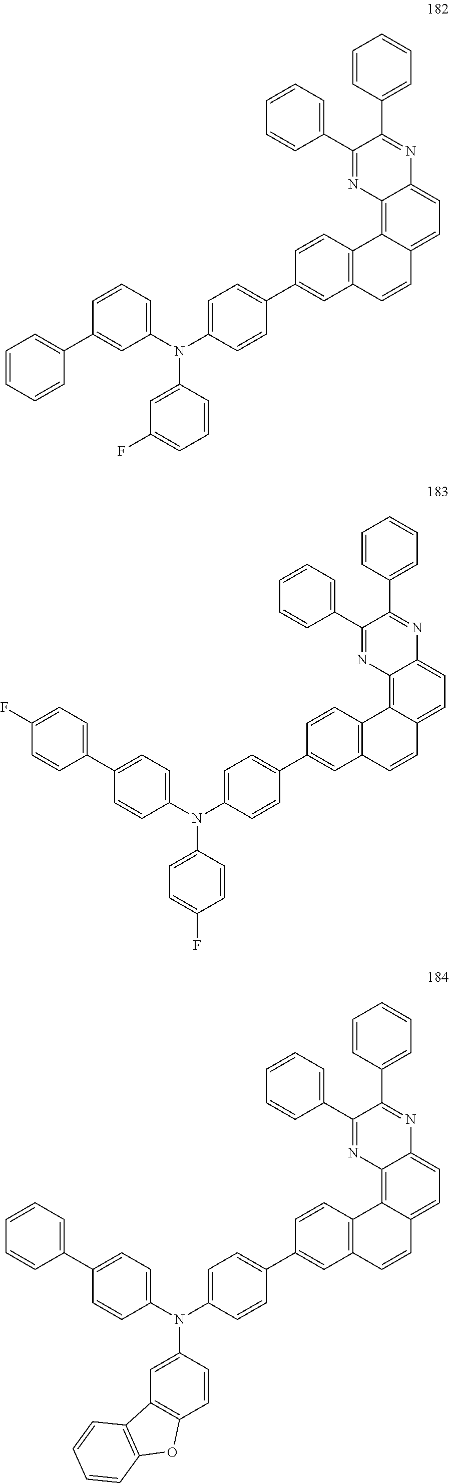 [SMILES: Fc1cccc(N(c2ccc(-c3ccc4c(ccc5ccc6nc(-c7ccccc7)c(-c7ccccc7)nc6c54)c3)cc2)c2cccc(-c3ccccc3)c2)c1 ; Fc1ccc(-c2ccc(N(c3ccc(F)cc3)c3ccc(-c4ccc5c(ccc6ccc7nc(-c8ccccc8)c(-c8ccccc8)nc7c65)c4)cc3)cc2)cc1 ; c1ccc(-c2ccc(N(c3ccc(-c4ccc5c(ccc6ccc7nc(-c8ccccc8)c(-c8ccccc8)nc7c65)c4)cc3)c3ccc4oc5ccccc5c4c3)cc2)cc1]

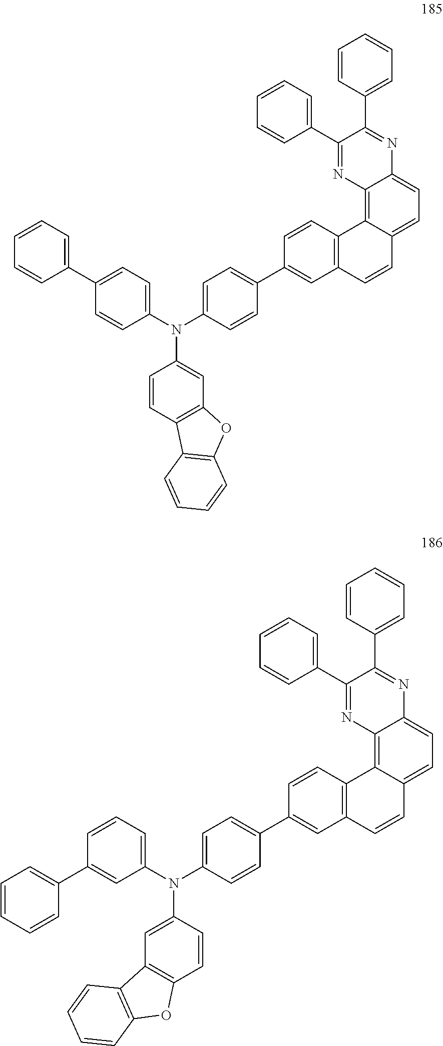 [SMILES: c1ccc(-c2ccc(N(c3ccc(-c4ccc5c(ccc6ccc7nc(-c8ccccc8)c(-c8ccccc8)nc7c65)c4)cc3)c3ccc4c(c3)oc3ccccc34)cc2)cc1 ; c1ccc(-c2cccc(N(c3ccc(-c4ccc5c(ccc6ccc7nc(-c8ccccc8)c(-c8ccccc8)nc7c65)c4)cc3)c3ccc4oc5ccccc5c4c3)c2)cc1]

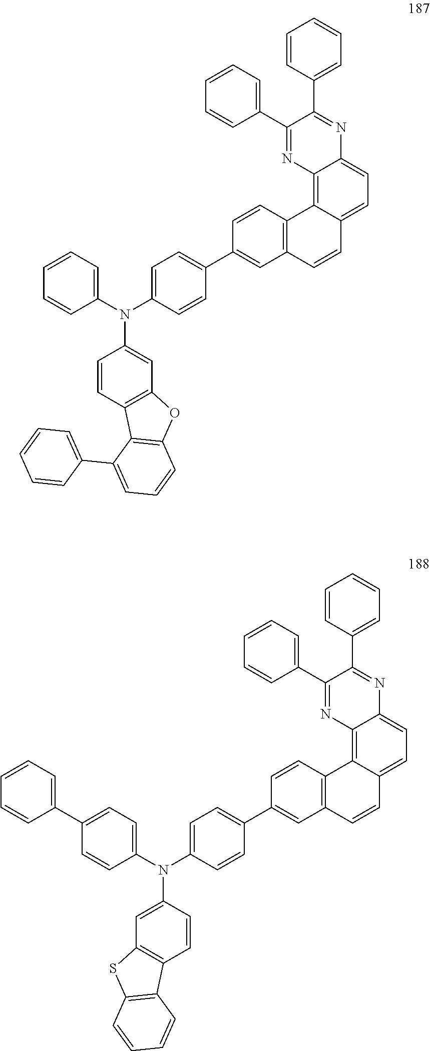 [SMILES: c1ccc(-c2nc3ccc4ccc5cc(-c6ccc(N(c7ccccc7)c7ccc8c(c7)oc7cccc(-c9ccccc9)c78)cc6)ccc5c4c3nc2-c2ccccc2)cc1 ; c1ccc(-c2ccc(N(c3ccc(-c4ccc5c(ccc6ccc7nc(-c8ccccc8)c(-c8ccccc8)nc7c65)c4)cc3)c3ccc4c(c3)sc3ccccc34)cc2)cc1]

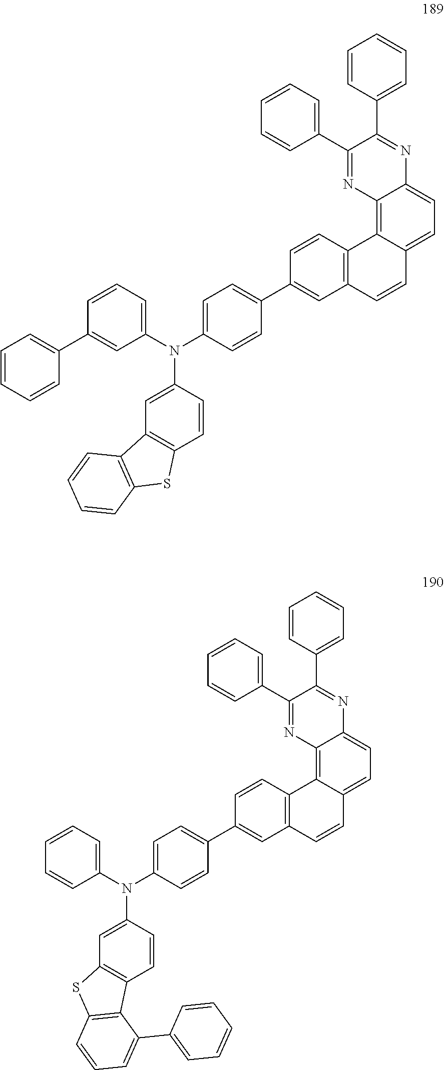 [SMILES: c1ccc(-c2cccc(N(c3ccc(-c4ccc5c(ccc6ccc7nc(-c8ccccc8)c(-c8ccccc8)nc7c65)c4)cc3)c3ccc4sc5ccccc5c4c3)c2)cc1 ; c1ccc(-c2nc3ccc4ccc5cc(-c6ccc(N(c7ccccc7)c7ccc8c(c7)sc7cccc(-c9ccccc9)c78)cc6)ccc5c4c3nc2-c2ccccc2)cc1]

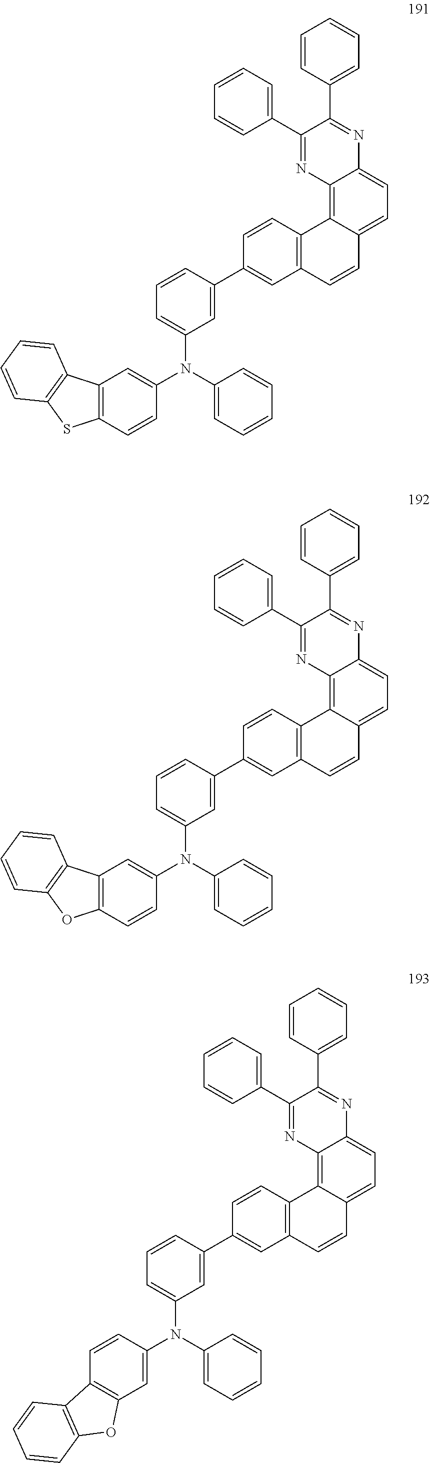 [SMILES: c1ccc(-c2nc3ccc4ccc5cc(-c6cccc(N(c7ccccc7)c7ccc8sc9ccccc9c8c7)c6)ccc5c4c3nc2-c2ccccc2)cc1 ; c1ccc(-c2nc3ccc4ccc5cc(-c6cccc(N(c7ccccc7)c7ccc8oc9ccccc9c8c7)c6)ccc5c4c3nc2-c2ccccc2)cc1 ; c1ccc(-c2nc3ccc4ccc5cc(-c6cccc(N(c7ccccc7)c7ccc8c(c7)oc7ccccc78)c6)ccc5c4c3nc2-c2ccccc2)cc1]

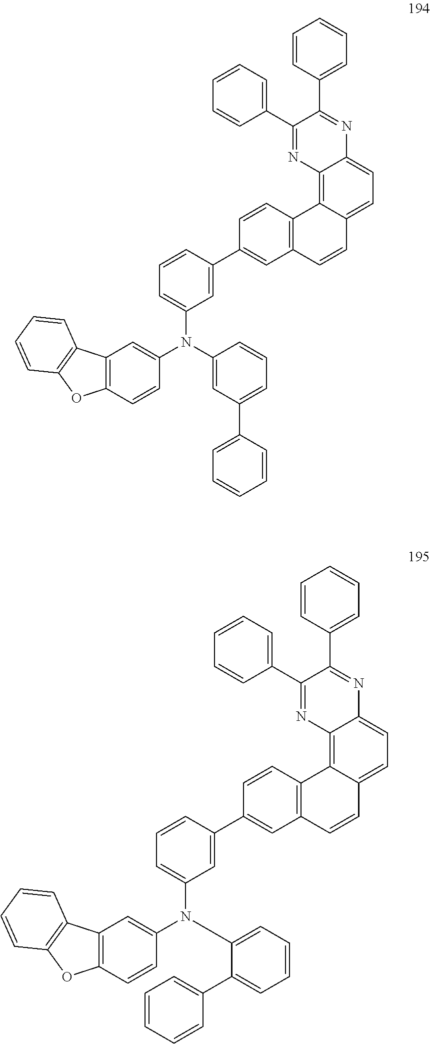 [SMILES: c1ccc(-c2cccc(N(c3cccc(-c4ccc5c(ccc6ccc7nc(-c8ccccc8)c(-c8ccccc8)nc7c65)c4)c3)c3ccc4oc5ccccc5c4c3)c2)cc1 ; c1ccc(-c2ccccc2N(c2cccc(-c3ccc4c(ccc5ccc6nc(-c7ccccc7)c(-c7ccccc7)nc6c54)c3)c2)c2ccc3oc4ccccc4c3c2)cc1]

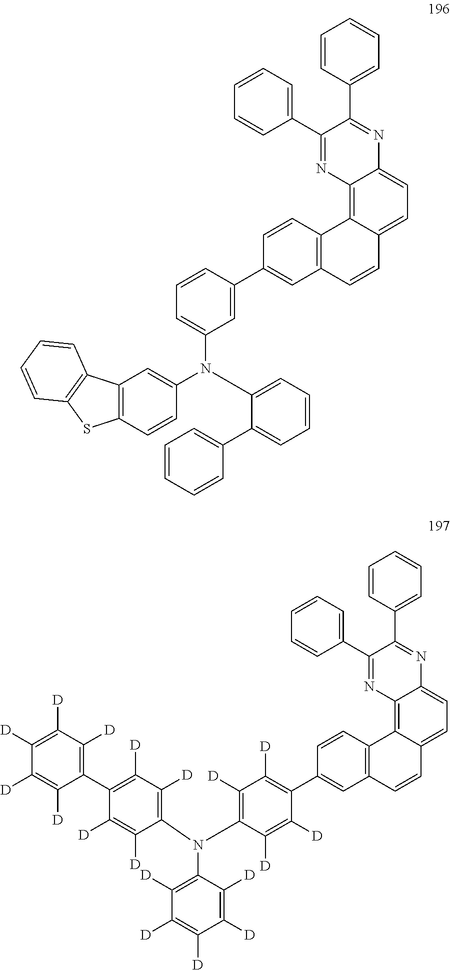 [SMILES: c1ccc(-c2ccccc2N(c2cccc(-c3ccc4c(ccc5ccc6nc(-c7ccccc7)c(-c7ccccc7)nc6c54)c3)c2)c2ccc3sc4ccccc4c3c2)cc1 ; [2H]c1c([2H])c([2H])c(-c2c([2H])c([2H])c(N(c3c([2H])c([2H])c([2H])c([2H])c3[2H])c3c([2H])c([2H])c(-c4ccc5c(ccc6ccc7nc(-c8ccccc8)c(-c8ccccc8)nc7c65)c4)c([2H])c3[2H])c([2H])c2[2H])c([2H])c1[2H]]

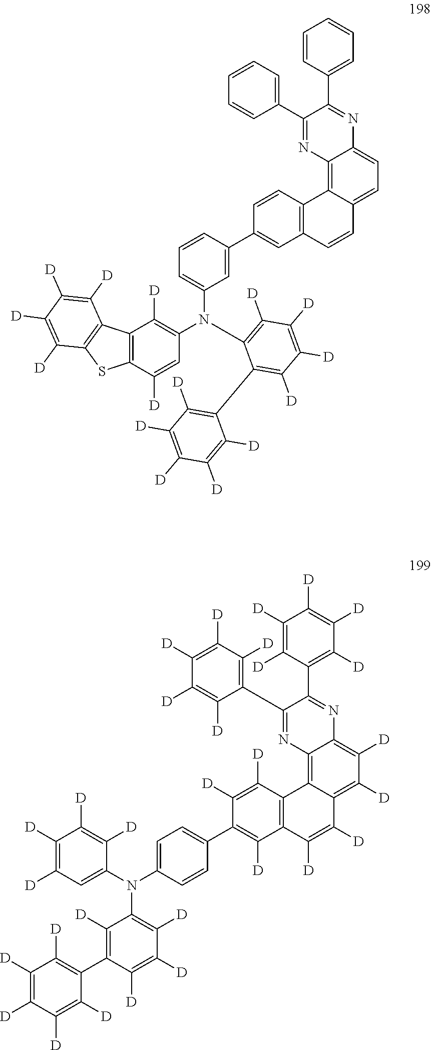 [SMILES: [2H]c1c([2H])c([2H])c(-c2c([2H])c([2H])c([2H])c([2H])c2N(c2cccc(-c3ccc4c(ccc5ccc6nc(-c7ccccc7)c(-c7ccccc7)nc6c54)c3)c2)c2cc([2H])c3sc4c([2H])c([2H])c([2H])c([2H])c4c3c2[2H])c([2H])c1[2H] ; [2H]c1cc(N(c2ccc(-c3c([2H])c([2H])c4c(c3[2H])c([2H])c([2H])c3c([2H])c([2H])c5nc(-c6c([2H])c([2H])c([2H])c([2H])c6[2H])c(-c6c([2H])c([2H])c([2H])c([2H])c6[2H])nc5c34)cc2)c2c([2H])c([2H])c([2H])c(-c3c([2H])c([2H])c([2H])c([2H])c3[2H])c2[2H])c([2H])c([2H])c1[2H]]

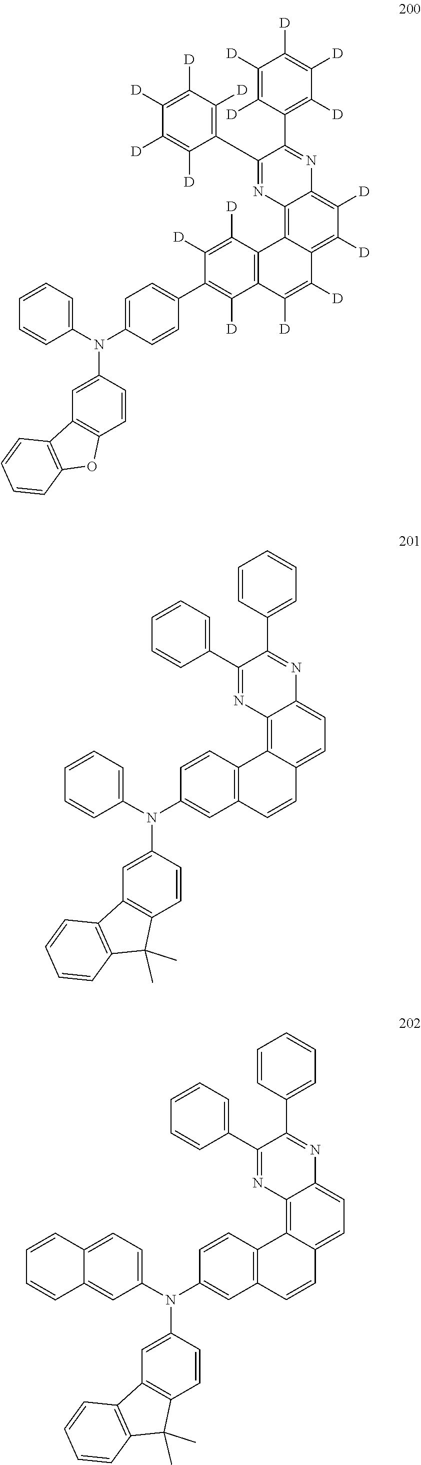 [SMILES: [2H]c1c([2H])c([2H])c(-c2nc3c([2H])c([2H])c4c([2H])c([2H])c5c([2H])c(-c6ccc(N(c7ccccc7)c7ccc8oc9ccccc9c8c7)cc6)c([2H])c([2H])c5c4c3nc2-c2c([2H])c([2H])c([2H])c([2H])c2[2H])c([2H])c1[2H] ; CC1(C)c2ccccc2-c2cc(N(c3ccccc3)c3ccc4c(ccc5ccc6nc(-c7ccccc7)c(-c7ccccc7)nc6c54)c3)ccc21 ; CC1(C)c2ccccc2-c2cc(N(c3ccc4ccccc4c3)c3ccc4c(ccc5ccc6nc(-c7ccccc7)c(-c7ccccc7)nc6c54)c3)ccc21]

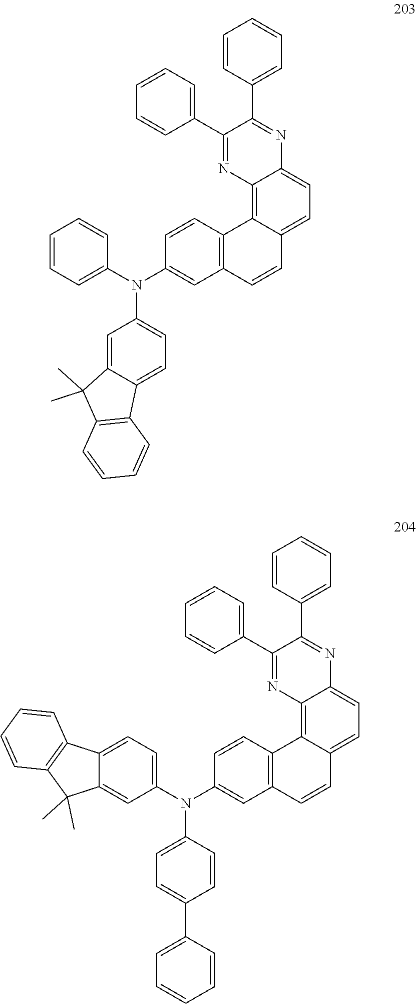 [SMILES: CC1(C)c2ccccc2-c2ccc(N(c3ccccc3)c3ccc4c(ccc5ccc6nc(-c7ccccc7)c(-c7ccccc7)nc6c54)c3)cc21 ; CC1(C)c2ccccc2-c2ccc(N(c3ccc(-c4ccccc4)cc3)c3ccc4c(ccc5ccc6nc(-c7ccccc7)c(-c7ccccc7)nc6c54)c3)cc21]

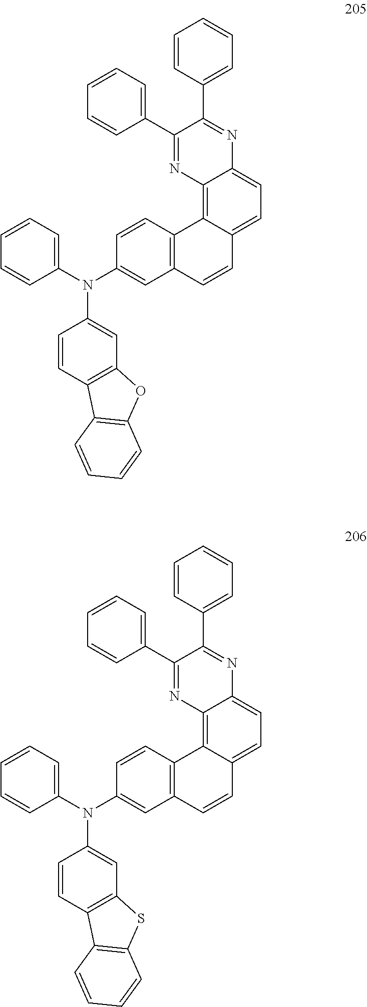 [SMILES: c1ccc(-c2nc3ccc4ccc5cc(N(c6ccccc6)c6ccc7c(c6)oc6ccccc67)ccc5c4c3nc2-c2ccccc2)cc1 ; c1ccc(-c2nc3ccc4ccc5cc(N(c6ccccc6)c6ccc7c(c6)sc6ccccc67)ccc5c4c3nc2-c2ccccc2)cc1]

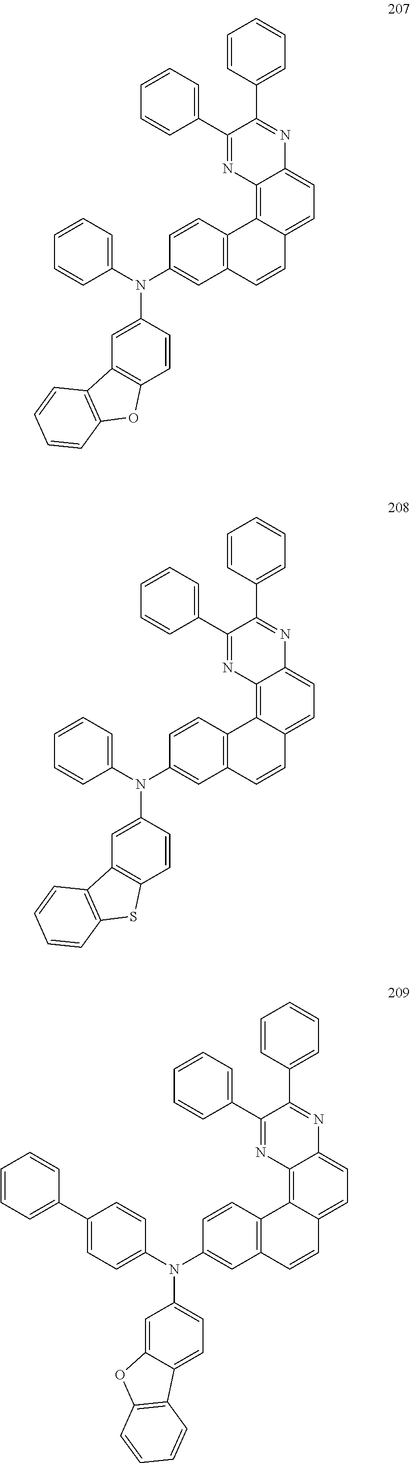 [SMILES: c1ccc(-c2nc3ccc4ccc5cc(N(c6ccccc6)c6ccc7oc8ccccc8c7c6)ccc5c4c3nc2-c2ccccc2)cc1 ; c1ccc(-c2nc3ccc4ccc5cc(N(c6ccccc6)c6ccc7sc8ccccc8c7c6)ccc5c4c3nc2-c2ccccc2)cc1 ; c1ccc(-c2ccc(N(c3ccc4c(ccc5ccc6nc(-c7ccccc7)c(-c7ccccc7)nc6c54)c3)c3ccc4c(c3)oc3ccccc34)cc2)cc1]

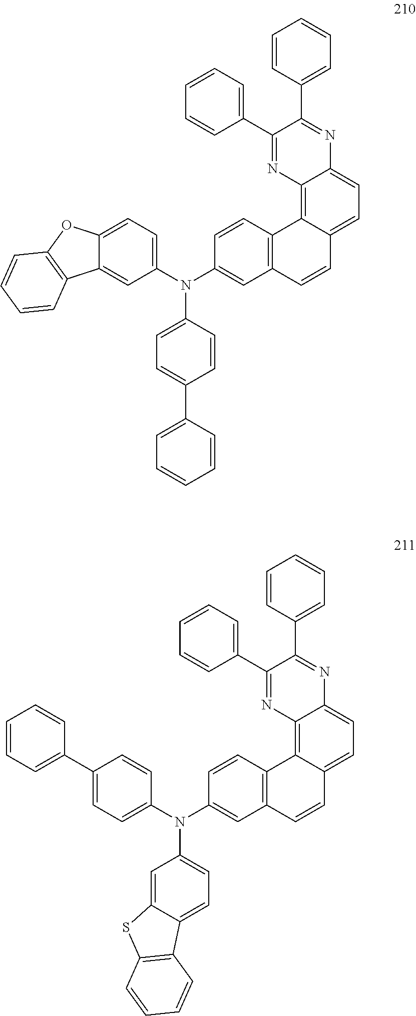 [SMILES: c1ccc(-c2ccc(N(c3ccc4c(ccc5ccc6nc(-c7ccccc7)c(-c7ccccc7)nc6c54)c3)c3ccc4oc5ccccc5c4c3)cc2)cc1 ; c1ccc(-c2ccc(N(c3ccc4c(ccc5ccc6nc(-c7ccccc7)c(-c7ccccc7)nc6c54)c3)c3ccc4c(c3)sc3ccccc34)cc2)cc1]

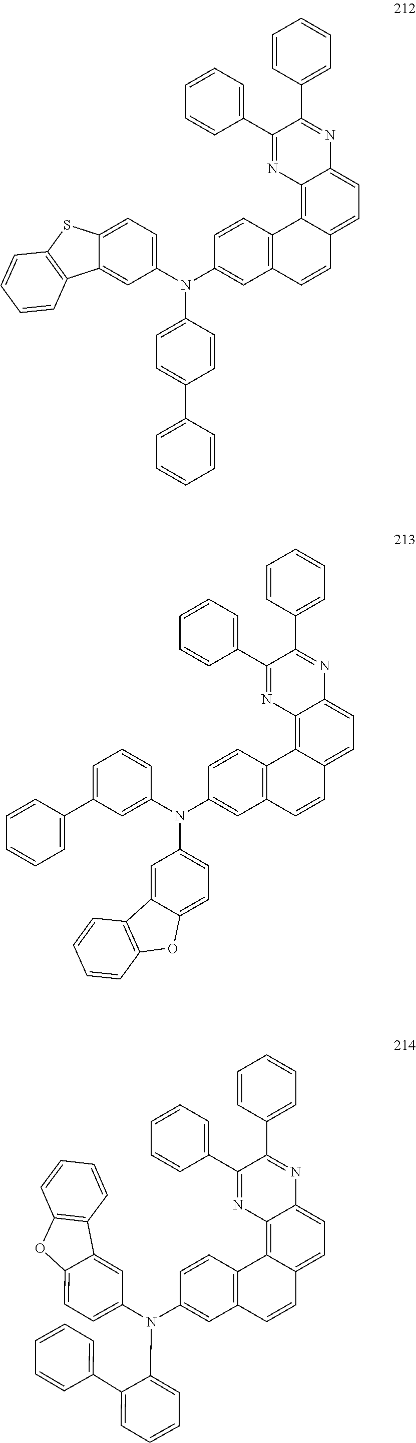 [SMILES: c1ccc(-c2ccc(N(c3ccc4c(ccc5ccc6nc(-c7ccccc7)c(-c7ccccc7)nc6c54)c3)c3ccc4sc5ccccc5c4c3)cc2)cc1 ; c1ccc(-c2cccc(N(c3ccc4c(ccc5ccc6nc(-c7ccccc7)c(-c7ccccc7)nc6c54)c3)c3ccc4oc5ccccc5c4c3)c2)cc1 ; c1ccc(-c2ccccc2N(c2ccc3c(ccc4ccc5nc(-c6ccccc6)c(-c6ccccc6)nc5c43)c2)c2ccc3oc4ccccc4c3c2)cc1]

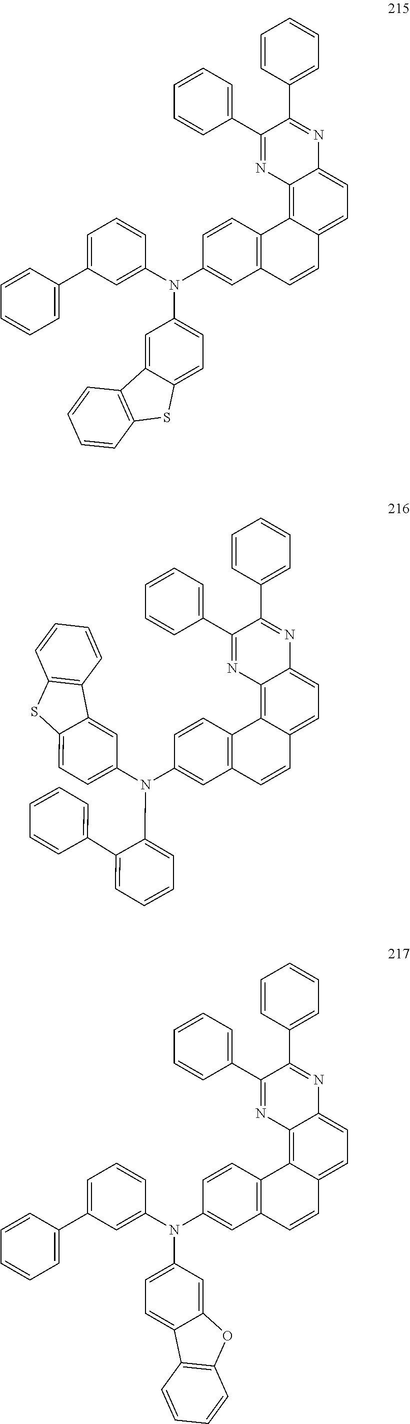 [SMILES: c1ccc(-c2cccc(N(c3ccc4c(ccc5ccc6nc(-c7ccccc7)c(-c7ccccc7)nc6c54)c3)c3ccc4sc5ccccc5c4c3)c2)cc1 ; c1ccc(-c2ccccc2N(c2ccc3c(ccc4ccc5nc(-c6ccccc6)c(-c6ccccc6)nc5c43)c2)c2ccc3sc4ccccc4c3c2)cc1 ; c1ccc(-c2cccc(N(c3ccc4c(ccc5ccc6nc(-c7ccccc7)c(-c7ccccc7)nc6c54)c3)c3ccc4c(c3)oc3ccccc34)c2)cc1]

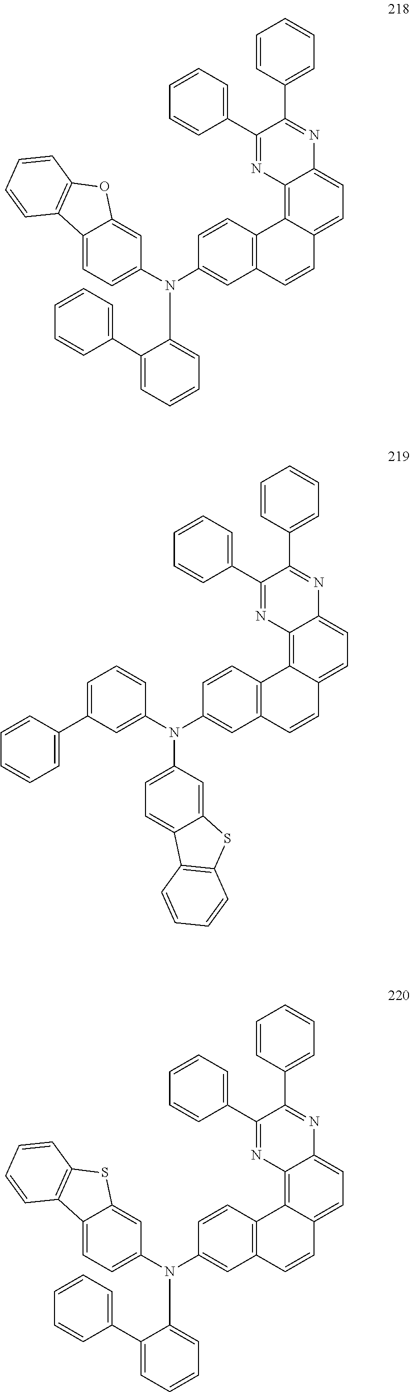 [SMILES: c1ccc(-c2ccccc2N(c2ccc3c(ccc4ccc5nc(-c6ccccc6)c(-c6ccccc6)nc5c43)c2)c2ccc3c(c2)oc2ccccc23)cc1 ; c1ccc(-c2cccc(N(c3ccc4c(ccc5ccc6nc(-c7ccccc7)c(-c7ccccc7)nc6c54)c3)c3ccc4c(c3)sc3ccccc34)c2)cc1 ; c1ccc(-c2ccccc2N(c2ccc3c(ccc4ccc5nc(-c6ccccc6)c(-c6ccccc6)nc5c43)c2)c2ccc3c(c2)sc2ccccc23)cc1]

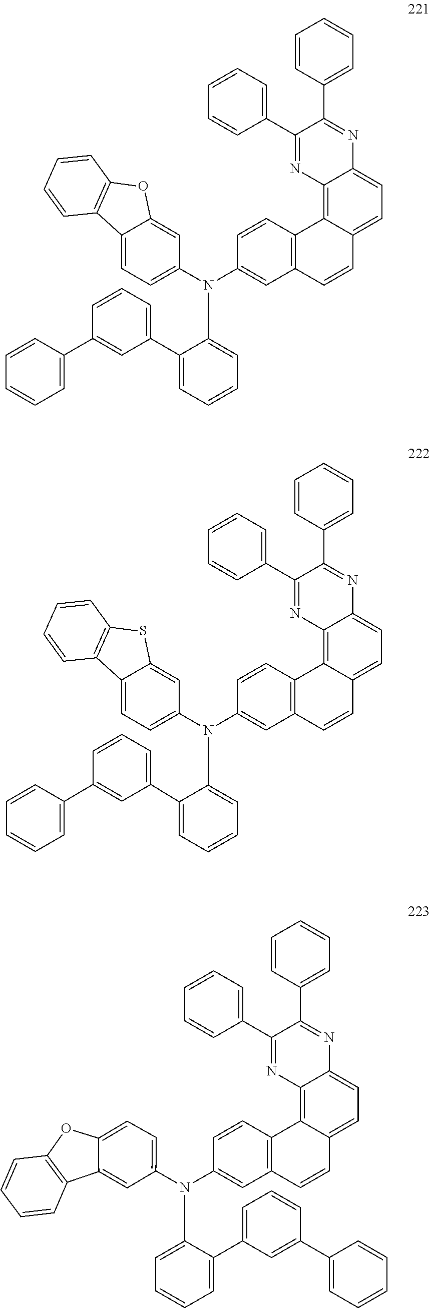 [SMILES: c1ccc(-c2cccc(-c3ccccc3N(c3ccc4c(ccc5ccc6nc(-c7ccccc7)c(-c7ccccc7)nc6c54)c3)c3ccc4c(c3)oc3ccccc34)c2)cc1 ; c1ccc(-c2cccc(-c3ccccc3N(c3ccc4c(ccc5ccc6nc(-c7ccccc7)c(-c7ccccc7)nc6c54)c3)c3ccc4c(c3)sc3ccccc34)c2)cc1 ; c1ccc(-c2cccc(-c3ccccc3N(c3ccc4c(ccc5ccc6nc(-c7ccccc7)c(-c7ccccc7)nc6c54)c3)c3ccc4oc5ccccc5c4c3)c2)cc1]

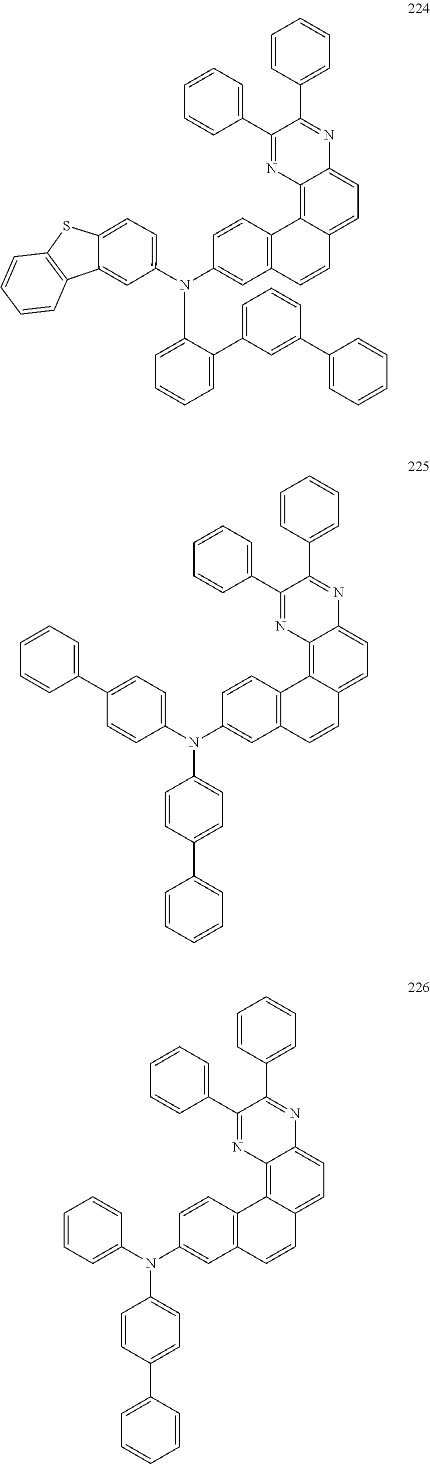 [SMILES: c1ccc(-c2cccc(-c3ccccc3N(c3ccc4c(ccc5ccc6nc(-c7ccccc7)c(-c7ccccc7)nc6c54)c3)c3ccc4sc5ccccc5c4c3)c2)cc1 ; c1ccc(-c2ccc(N(c3ccc(-c4ccccc4)cc3)c3ccc4c(ccc5ccc6nc(-c7ccccc7)c(-c7ccccc7)nc6c54)c3)cc2)cc1 ; c1ccc(-c2ccc(N(c3ccccc3)c3ccc4c(ccc5ccc6nc(-c7ccccc7)c(-c7ccccc7)nc6c54)c3)cc2)cc1]

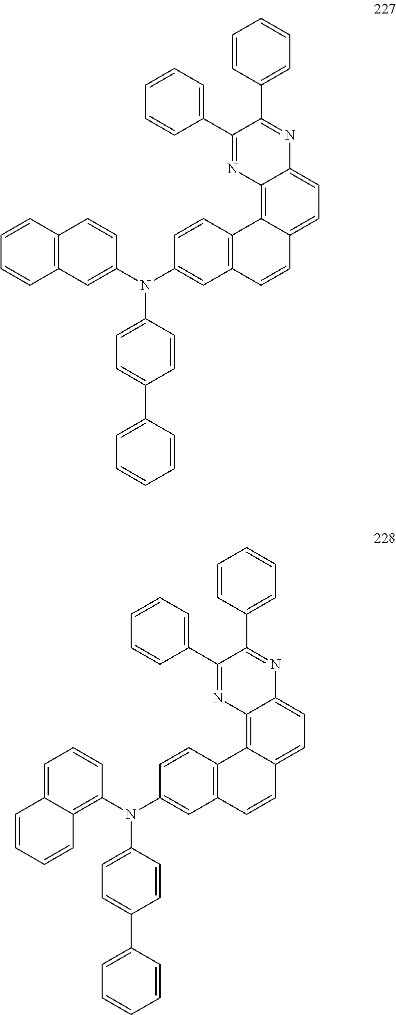 [SMILES: c1ccc(-c2ccc(N(c3ccc4ccccc4c3)c3ccc4c(ccc5ccc6nc(-c7ccccc7)c(-c7ccccc7)nc6c54)c3)cc2)cc1 ; c1ccc(-c2ccc(N(c3ccc4c(ccc5ccc6nc(-c7ccccc7)c(-c7ccccc7)nc6c54)c3)c3cccc4ccccc34)cc2)cc1]

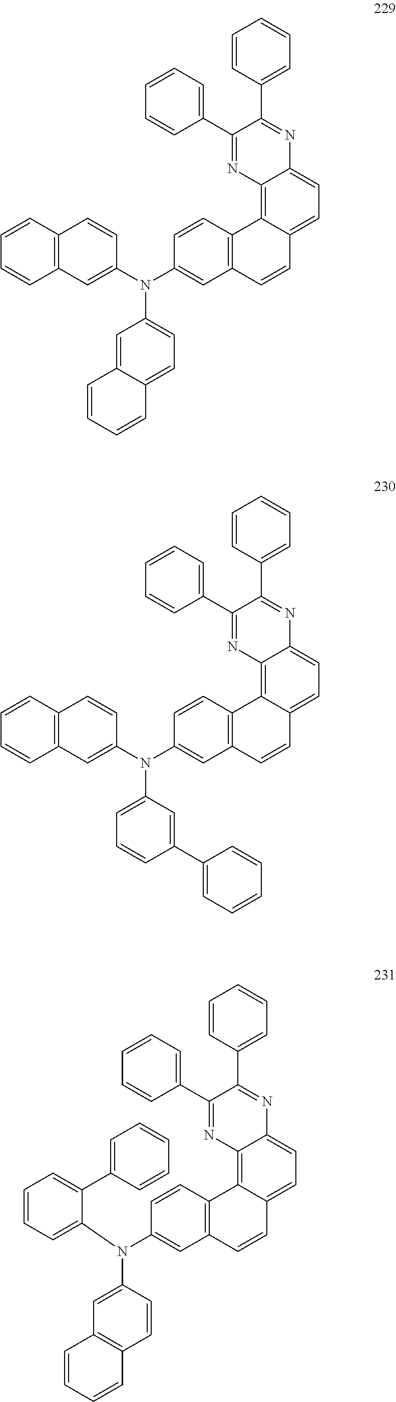 [SMILES: c1ccc(-c2nc3ccc4ccc5cc(N(c6ccc7ccccc7c6)c6ccc7ccccc7c6)ccc5c4c3nc2-c2ccccc2)cc1 ; c1ccc(-c2cccc(N(c3ccc4ccccc4c3)c3ccc4c(ccc5ccc6nc(-c7ccccc7)c(-c7ccccc7)nc6c54)c3)c2)cc1 ; c1ccc(-c2ccccc2N(c2ccc3ccccc3c2)c2ccc3c(ccc4ccc5nc(-c6ccccc6)c(-c6ccccc6)nc5c43)c2)cc1]

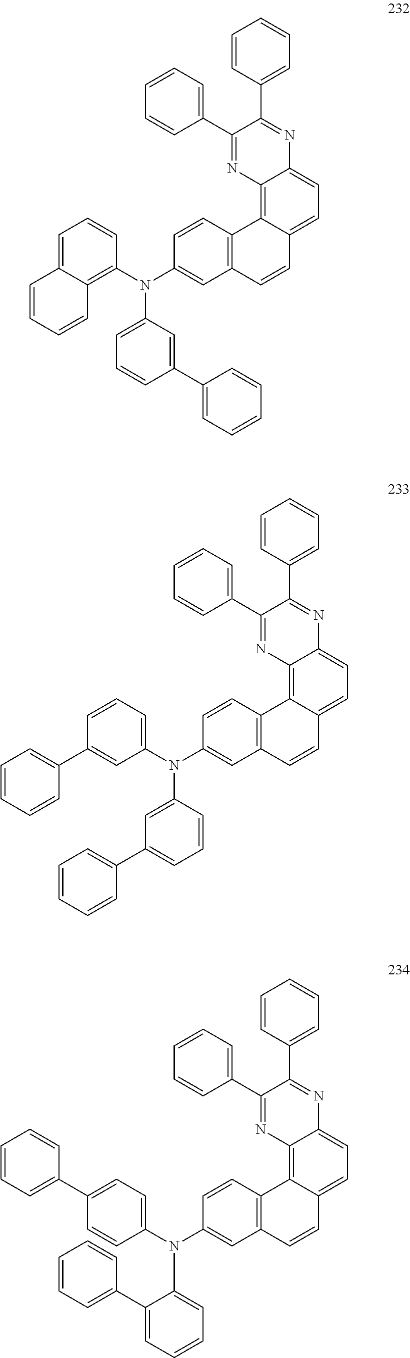 [SMILES: c1ccc(-c2cccc(N(c3ccc4c(ccc5ccc6nc(-c7ccccc7)c(-c7ccccc7)nc6c54)c3)c3cccc4ccccc34)c2)cc1 ; c1ccc(-c2cccc(N(c3cccc(-c4ccccc4)c3)c3ccc4c(ccc5ccc6nc(-c7ccccc7)c(-c7ccccc7)nc6c54)c3)c2)cc1 ; c1ccc(-c2ccc(N(c3ccc4c(ccc5ccc6nc(-c7ccccc7)c(-c7ccccc7)nc6c54)c3)c3ccccc3-c3ccccc3)cc2)cc1]

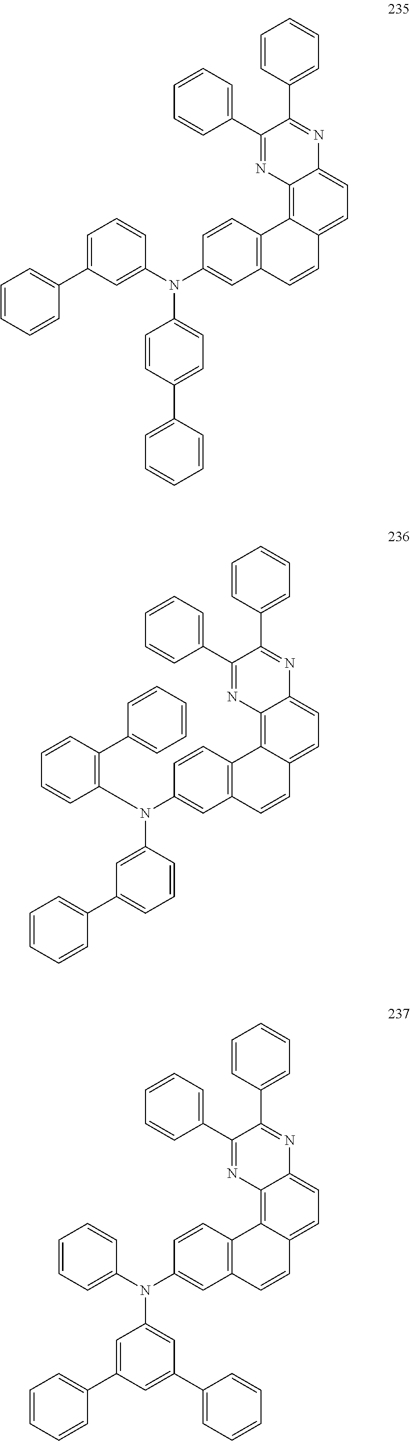 [SMILES: c1ccc(-c2ccc(N(c3cccc(-c4ccccc4)c3)c3ccc4c(ccc5ccc6nc(-c7ccccc7)c(-c7ccccc7)nc6c54)c3)cc2)cc1 ; c1ccc(-c2cccc(N(c3ccc4c(ccc5ccc6nc(-c7ccccc7)c(-c7ccccc7)nc6c54)c3)c3ccccc3-c3ccccc3)c2)cc1 ; c1ccc(-c2cc(-c3ccccc3)cc(N(c3ccccc3)c3ccc4c(ccc5ccc6nc(-c7ccccc7)c(-c7ccccc7)nc6c54)c3)c2)cc1]

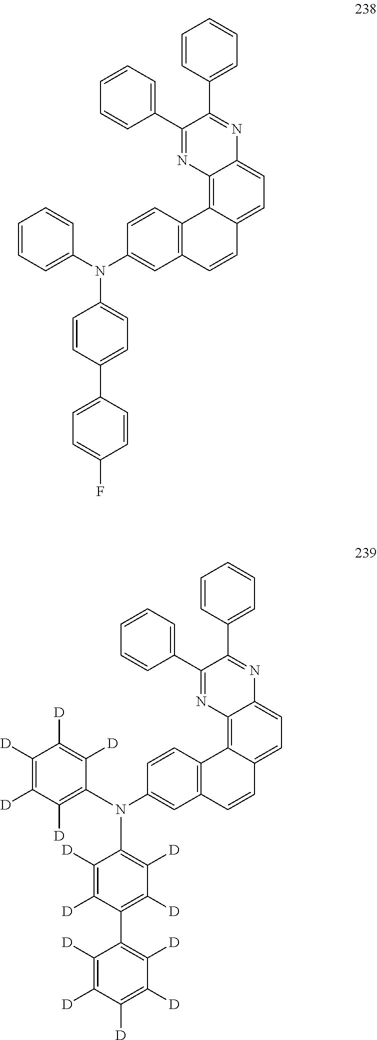 [SMILES: Fc1ccc(-c2ccc(N(c3ccccc3)c3ccc4c(ccc5ccc6nc(-c7ccccc7)c(-c7ccccc7)nc6c54)c3)cc2)cc1 ; [2H]c1c([2H])c([2H])c(-c2c([2H])c([2H])c(N(c3ccc4c(ccc5ccc6nc(-c7ccccc7)c(-c7ccccc7)nc6c54)c3)c3c([2H])c([2H])c([2H])c([2H])c3[2H])c([2H])c2[2H])c([2H])c1[2H]]

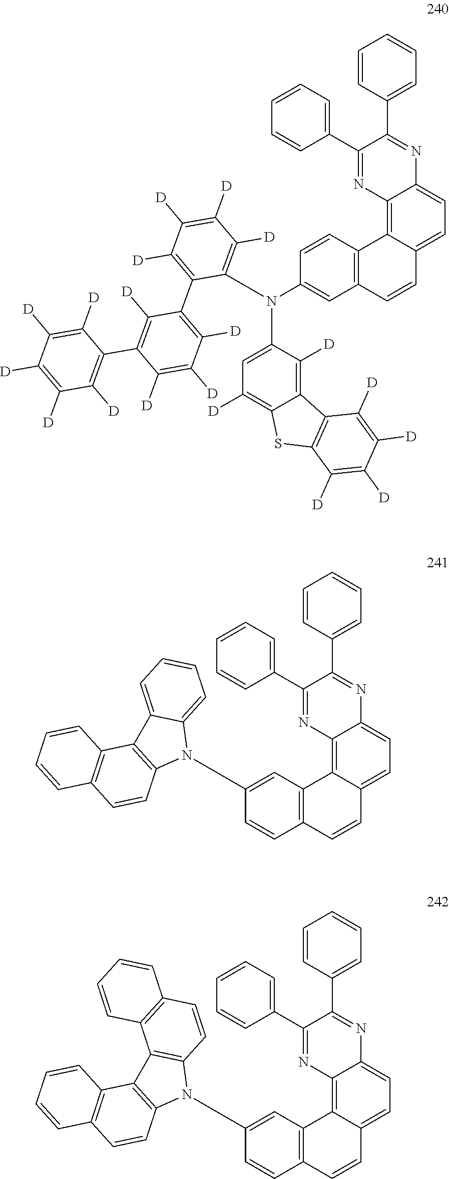 [SMILES: [2H]c1c([2H])c([2H])c(-c2c([2H])c([2H])c([2H])c(-c3c([2H])c([2H])c([2H])c([2H])c3N(c3ccc4c(ccc5ccc6nc(-c7ccccc7)c(-c7ccccc7)nc6c54)c3)c3cc([2H])c4sc5c([2H])c([2H])c([2H])c([2H])c5c4c3[2H])c2[2H])c([2H])c1[2H] ; c1ccc(-c2nc3ccc4ccc5ccc(-n6c7ccccc7c7c8ccccc8ccc76)cc5c4c3nc2-c2ccccc2)cc1 ; c1ccc(-c2nc3ccc4ccc5ccc(-n6c7ccc8ccccc8c7c7c8ccccc8ccc76)cc5c4c3nc2-c2ccccc2)cc1]

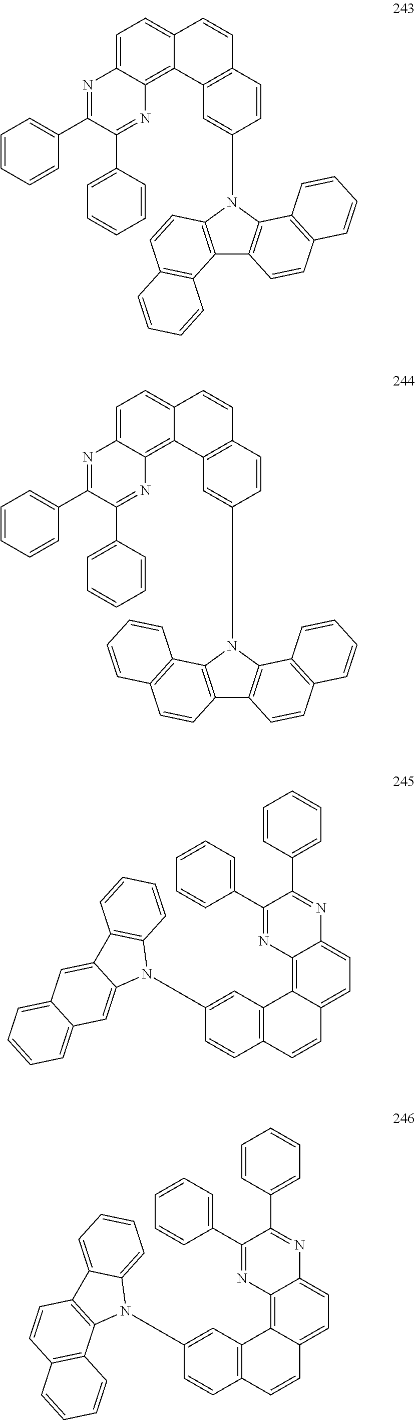 [SMILES: c1ccc(-c2nc3ccc4ccc5ccc(-n6c7ccc8ccccc8c7c7ccc8ccccc8c76)cc5c4c3nc2-c2ccccc2)cc1 ; c1ccc(-c2nc3ccc4ccc5ccc(-n6c7c8ccccc8ccc7c7ccc8ccccc8c76)cc5c4c3nc2-c2ccccc2)cc1 ; c1ccc(-c2nc3ccc4ccc5ccc(-n6c7ccccc7c7cc8ccccc8cc76)cc5c4c3nc2-c2ccccc2)cc1 ; c1ccc(-c2nc3ccc4ccc5ccc(-n6c7ccccc7c7ccc8ccccc8c76)cc5c4c3nc2-c2ccccc2)cc1]

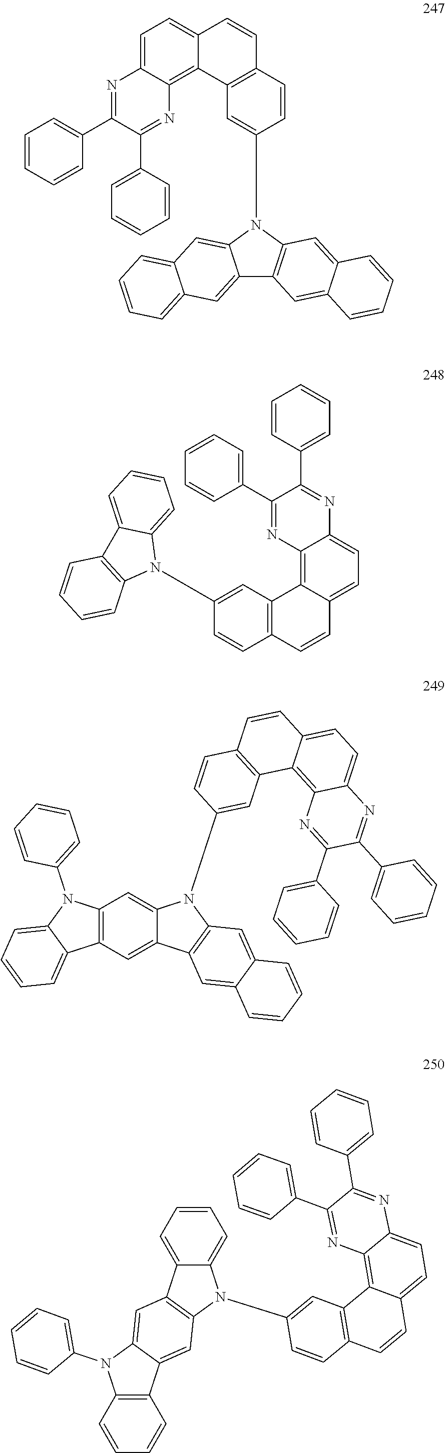 [SMILES: c1ccc(-c2nc3ccc4ccc5ccc(-n6c7cc8ccccc8cc7c7cc8ccccc8cc76)cc5c4c3nc2-c2ccccc2)cc1 ; c1ccc(-c2nc3ccc4ccc5ccc(-n6c7ccccc7c7ccccc76)cc5c4c3nc2-c2ccccc2)cc1 ; c1ccc(-c2nc3ccc4ccc5ccc(-n6c7cc8ccccc8cc7c7cc8c9ccccc9n(-c9ccccc9)c8cc76)cc5c4c3nc2-c2ccccc2)cc1 ; c1ccc(-c2nc3ccc4ccc5ccc(-n6c7ccccc7c7cc8c(cc76)c6ccccc6n8-c6ccccc6)cc5c4c3nc2-c2ccccc2)cc1]

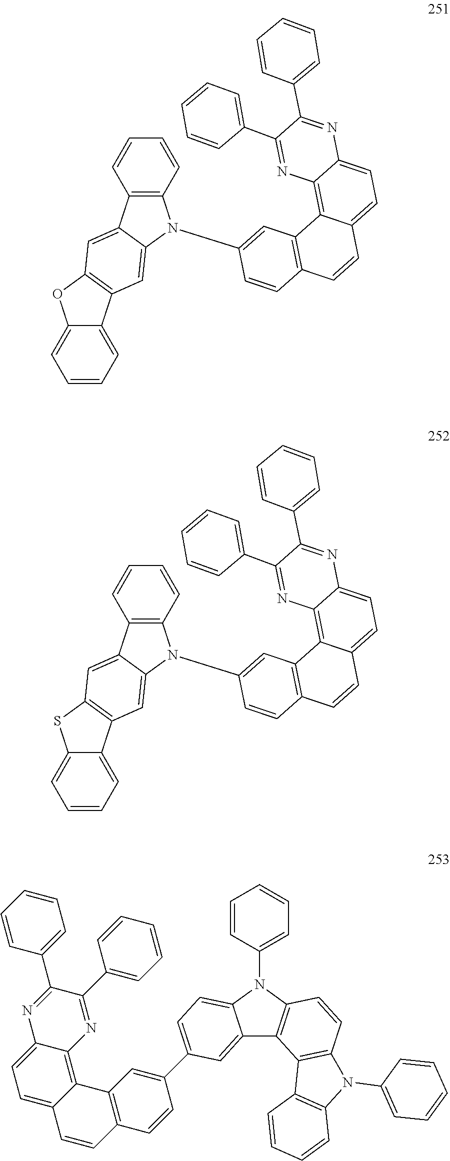 [SMILES: c1ccc(-c2nc3ccc4ccc5ccc(-n6c7ccccc7c7cc8oc9ccccc9c8cc76)cc5c4c3nc2-c2ccccc2)cc1 ; c1ccc(-c2nc3ccc4ccc5ccc(-n6c7ccccc7c7cc8sc9ccccc9c8cc76)cc5c4c3nc2-c2ccccc2)cc1 ; c1ccc(-c2nc3ccc4ccc5ccc(-c6ccc7c(c6)c6c8c9ccccc9n(-c9ccccc9)c8ccc6n7-c6ccccc6)cc5c4c3nc2-c2ccccc2)cc1]

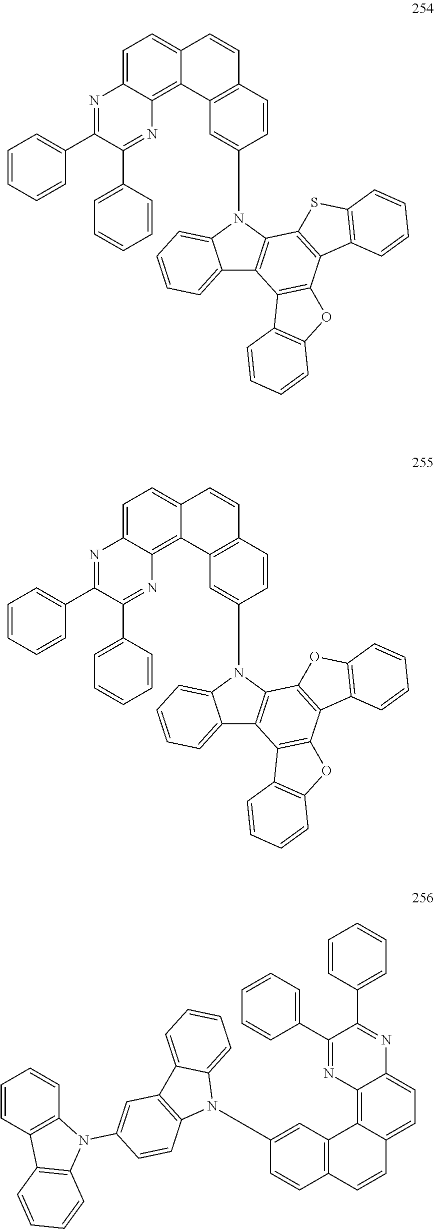 [SMILES: c1ccc(-c2nc3ccc4ccc5ccc(-n6c7ccccc7c7c8c9ccccc9oc8c8c9ccccc9sc8c76)cc5c4c3nc2-c2ccccc2)cc1 ; c1ccc(-c2nc3ccc4ccc5ccc(-n6c7ccccc7c7c8c9ccccc9oc8c8c9ccccc9oc8c76)cc5c4c3nc2-c2ccccc2)cc1 ; c1ccc(-c2nc3ccc4ccc5ccc(-n6c7ccccc7c7cc(-n8c9ccccc9c9ccccc98)ccc76)cc5c4c3nc2-c2ccccc2)cc1]

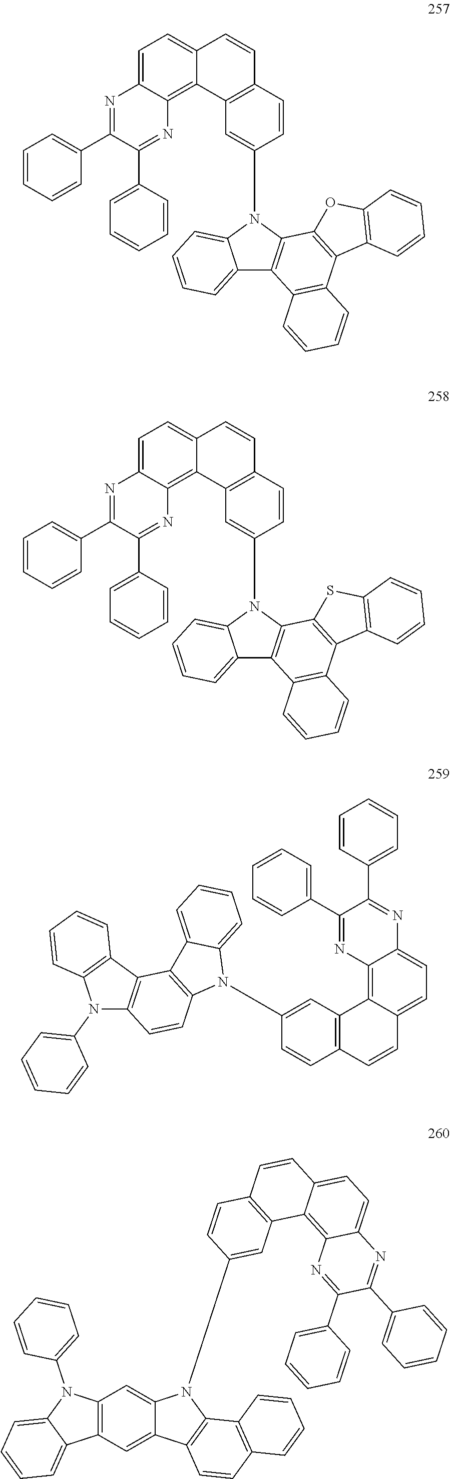 [SMILES: c1ccc(-c2nc3ccc4ccc5ccc(-n6c7ccccc7c7c8ccccc8c8c9ccccc9oc8c76)cc5c4c3nc2-c2ccccc2)cc1 ; c1ccc(-c2nc3ccc4ccc5ccc(-n6c7ccccc7c7c8ccccc8c8c9ccccc9sc8c76)cc5c4c3nc2-c2ccccc2)cc1 ; c1ccc(-c2nc3ccc4ccc5ccc(-n6c7ccccc7c7c8c9ccccc9n(-c9ccccc9)c8ccc76)cc5c4c3nc2-c2ccccc2)cc1 ; c1ccc(-c2nc3ccc4ccc5ccc(-n6c7cc8c(cc7c7ccc9ccccc9c76)c6ccccc6n8-c6ccccc6)cc5c4c3nc2-c2ccccc2)cc1]

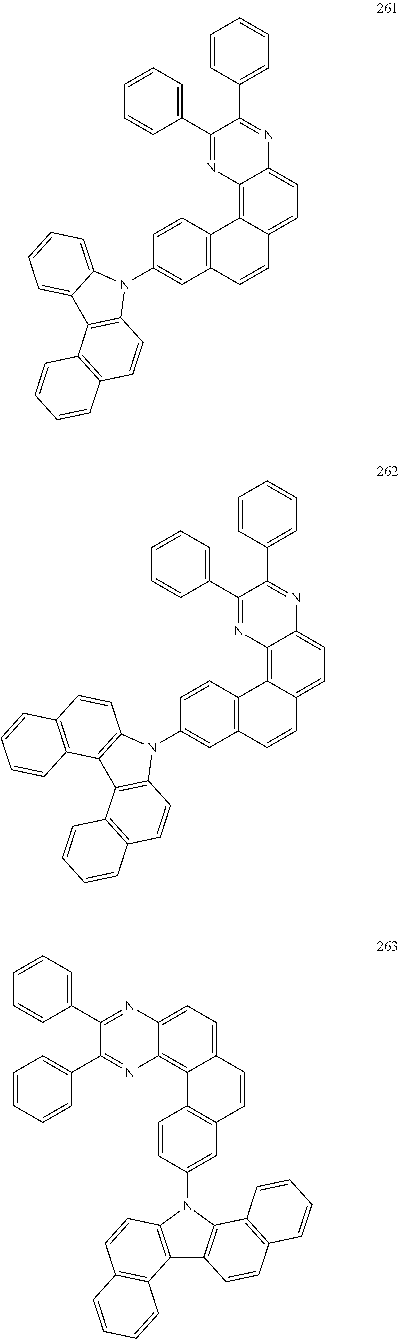 [SMILES: c1ccc(-c2nc3ccc4ccc5cc(-n6c7ccccc7c7c8ccccc8ccc76)ccc5c4c3nc2-c2ccccc2)cc1 ; c1ccc(-c2nc3ccc4ccc5cc(-n6c7ccc8ccccc8c7c7c8ccccc8ccc76)ccc5c4c3nc2-c2ccccc2)cc1 ; c1ccc(-c2nc3ccc4ccc5cc(-n6c7ccc8ccccc8c7c7ccc8ccccc8c76)ccc5c4c3nc2-c2ccccc2)cc1]

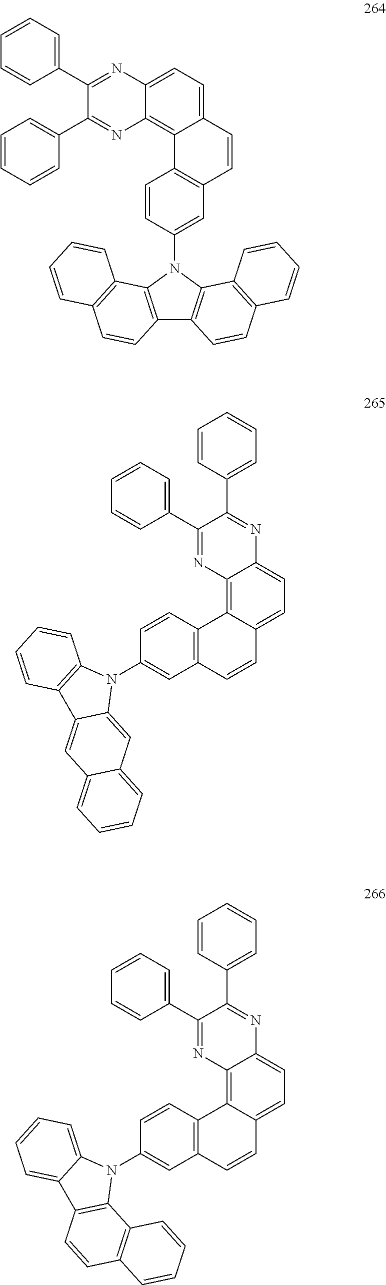 [SMILES: c1ccc(-c2nc3ccc4ccc5cc(-n6c7c8ccccc8ccc7c7ccc8ccccc8c76)ccc5c4c3nc2-c2ccccc2)cc1 ; c1ccc(-c2nc3ccc4ccc5cc(-n6c7ccccc7c7cc8ccccc8cc76)ccc5c4c3nc2-c2ccccc2)cc1 ; c1ccc(-c2nc3ccc4ccc5cc(-n6c7ccccc7c7ccc8ccccc8c76)ccc5c4c3nc2-c2ccccc2)cc1]

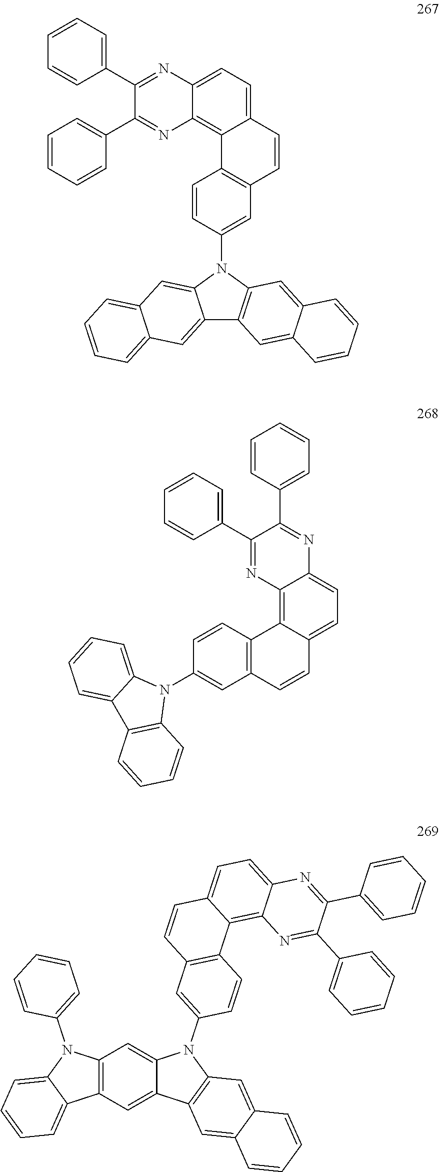 [SMILES: c1ccc(-c2nc3ccc4ccc5cc(-n6c7cc8ccccc8cc7c7cc8ccccc8cc76)ccc5c4c3nc2-c2ccccc2)cc1 ; c1ccc(-c2nc3ccc4ccc5cc(-n6c7ccccc7c7ccccc76)ccc5c4c3nc2-c2ccccc2)cc1 ; c1ccc(-c2nc3ccc4ccc5cc(-n6c7cc8ccccc8cc7c7cc8c9ccccc9n(-c9ccccc9)c8cc76)ccc5c4c3nc2-c2ccccc2)cc1]

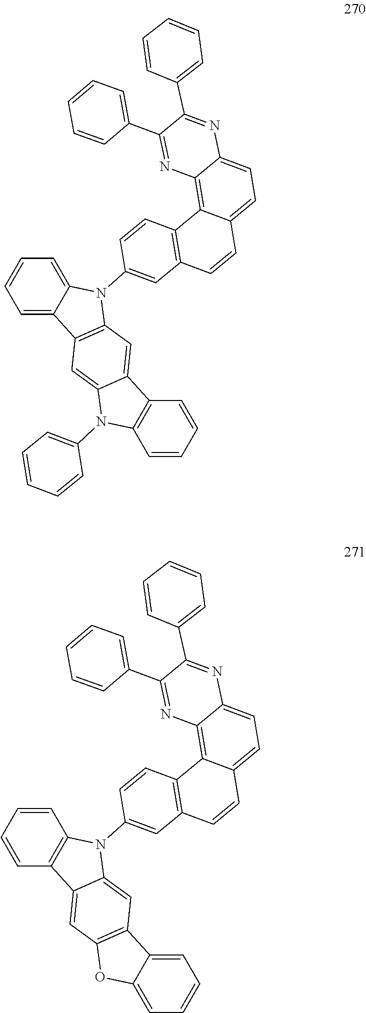 [SMILES: c1ccc(-c2nc3ccc4ccc5cc(-n6c7ccccc7c7cc8c(cc76)c6ccccc6n8-c6ccccc6)ccc5c4c3nc2-c2ccccc2)cc1 ; c1ccc(-c2nc3ccc4ccc5cc(-n6c7ccccc7c7cc8oc9ccccc9c8cc76)ccc5c4c3nc2-c2ccccc2)cc1]

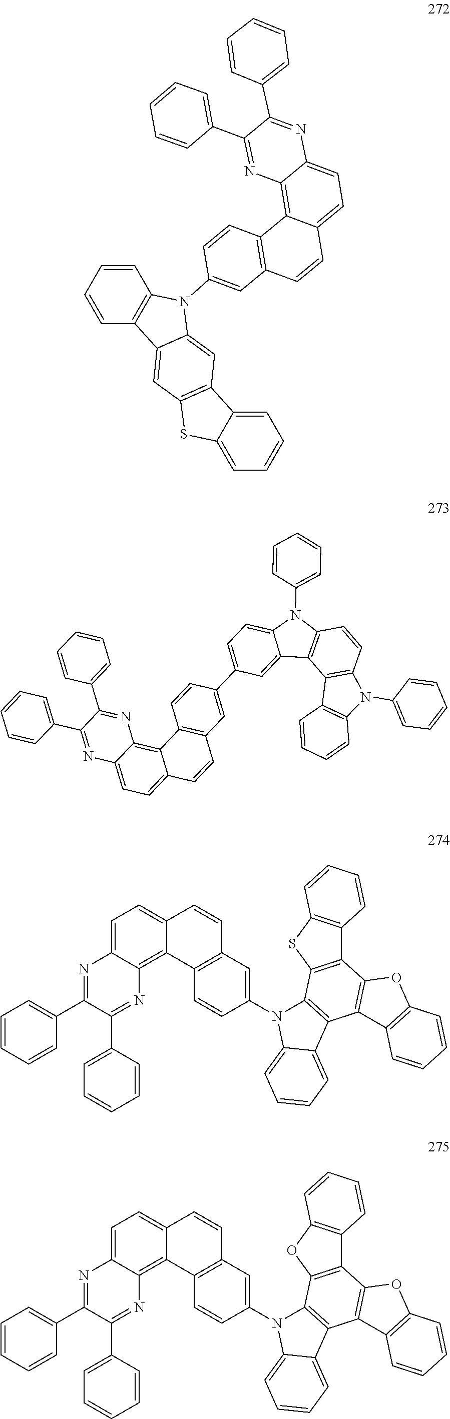 [SMILES: c1ccc(-c2nc3ccc4ccc5cc(-n6c7ccccc7c7cc8sc9ccccc9c8cc76)ccc5c4c3nc2-c2ccccc2)cc1 ; c1ccc(-c2nc3ccc4ccc5cc(-c6ccc7c(c6)c6c8c9ccccc9n(-c9ccccc9)c8ccc6n7-c6ccccc6)ccc5c4c3nc2-c2ccccc2)cc1 ; c1ccc(-c2nc3ccc4ccc5cc(-n6c7ccccc7c7c8c9ccccc9oc8c8c9ccccc9sc8c76)ccc5c4c3nc2-c2ccccc2)cc1 ; c1ccc(-c2nc3ccc4ccc5cc(-n6c7ccccc7c7c8c9ccccc9oc8c8c9ccccc9oc8c76)ccc5c4c3nc2-c2ccccc2)cc1]

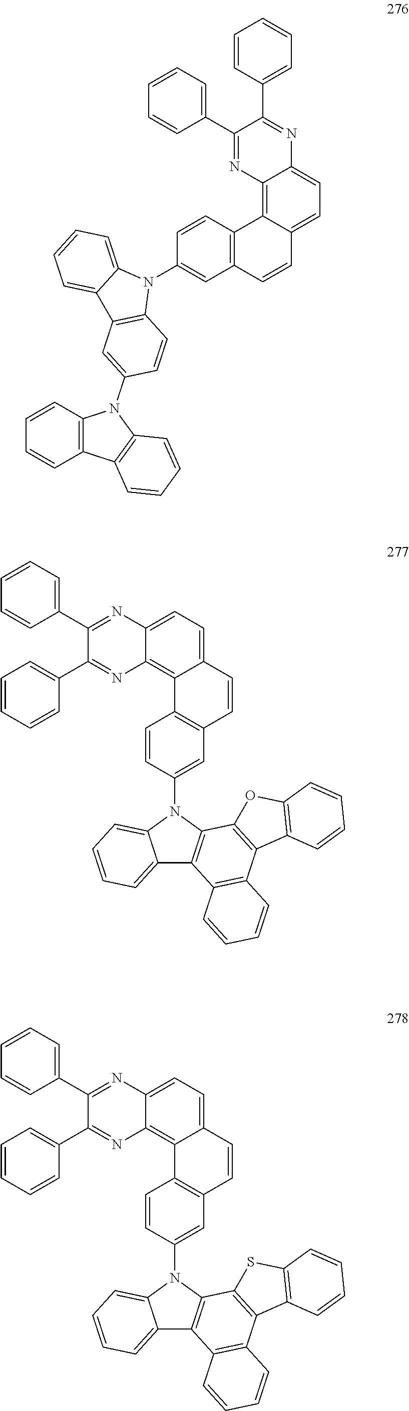 [SMILES: c1ccc(-c2nc3ccc4ccc5cc(-n6c7ccccc7c7cc(-n8c9ccccc9c9ccccc98)ccc76)ccc5c4c3nc2-c2ccccc2)cc1 ; c1ccc(-c2nc3ccc4ccc5cc(-n6c7ccccc7c7c8ccccc8c8c9ccccc9oc8c76)ccc5c4c3nc2-c2ccccc2)cc1 ; c1ccc(-c2nc3ccc4ccc5cc(-n6c7ccccc7c7c8ccccc8c8c9ccccc9sc8c76)ccc5c4c3nc2-c2ccccc2)cc1]

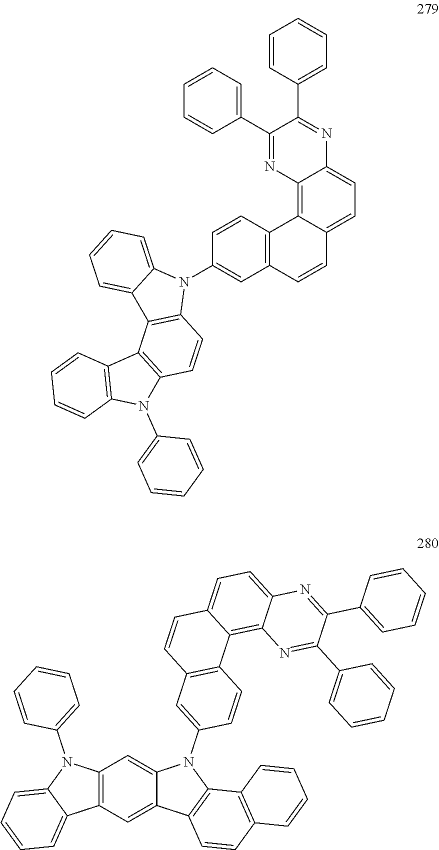 [SMILES: c1ccc(-c2nc3ccc4ccc5cc(-n6c7ccccc7c7c8c9ccccc9n(-c9ccccc9)c8ccc76)ccc5c4c3nc2-c2ccccc2)cc1 ; c1ccc(-c2nc3ccc4ccc5cc(-n6c7cc8c(cc7c7ccc9ccccc9c76)c6ccccc6n8-c6ccccc6)ccc5c4c3nc2-c2ccccc2)cc1]

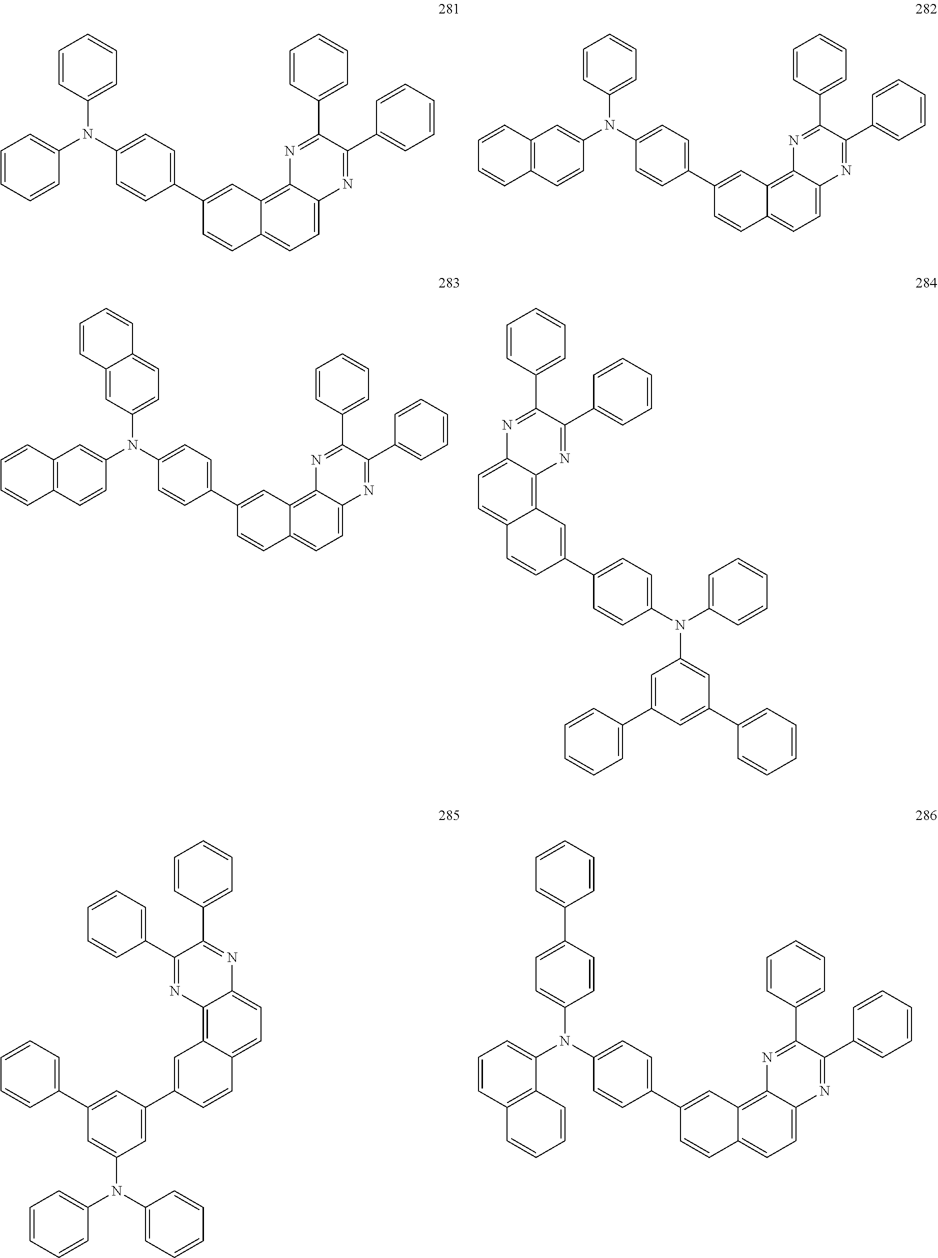

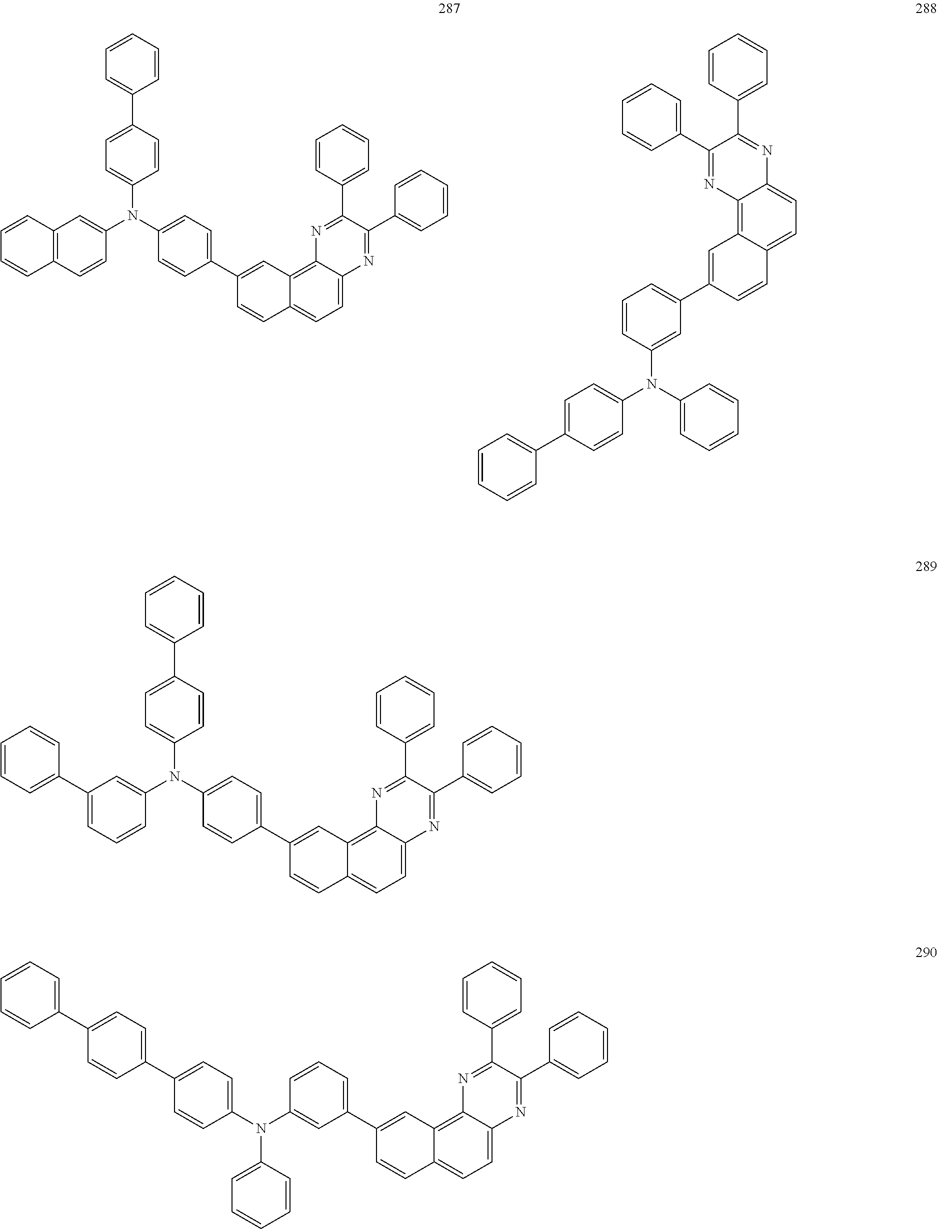 [SMILES: c1ccc(-c2ccc(N(c3ccc(-c4ccc5ccc6nc(-c7ccccc7)c(-c7ccccc7)nc6c5c4)cc3)c3ccc4ccccc4c3)cc2)cc1 ; c1ccc(-c2ccc(N(c3ccccc3)c3cccc(-c4ccc5ccc6nc(-c7ccccc7)c(-c7ccccc7)nc6c5c4)c3)cc2)cc1 ; c1ccc(-c2ccc(N(c3ccc(-c4ccc5ccc6nc(-c7ccccc7)c(-c7ccccc7)nc6c5c4)cc3)c3cccc(-c4ccccc4)c3)cc2)cc1 ; c1ccc(-c2ccc(-c3ccc(N(c4ccccc4)c4cccc(-c5ccc6ccc7nc(-c8ccccc8)c(-c8ccccc8)nc7c6c5)c4)cc3)cc2)cc1]

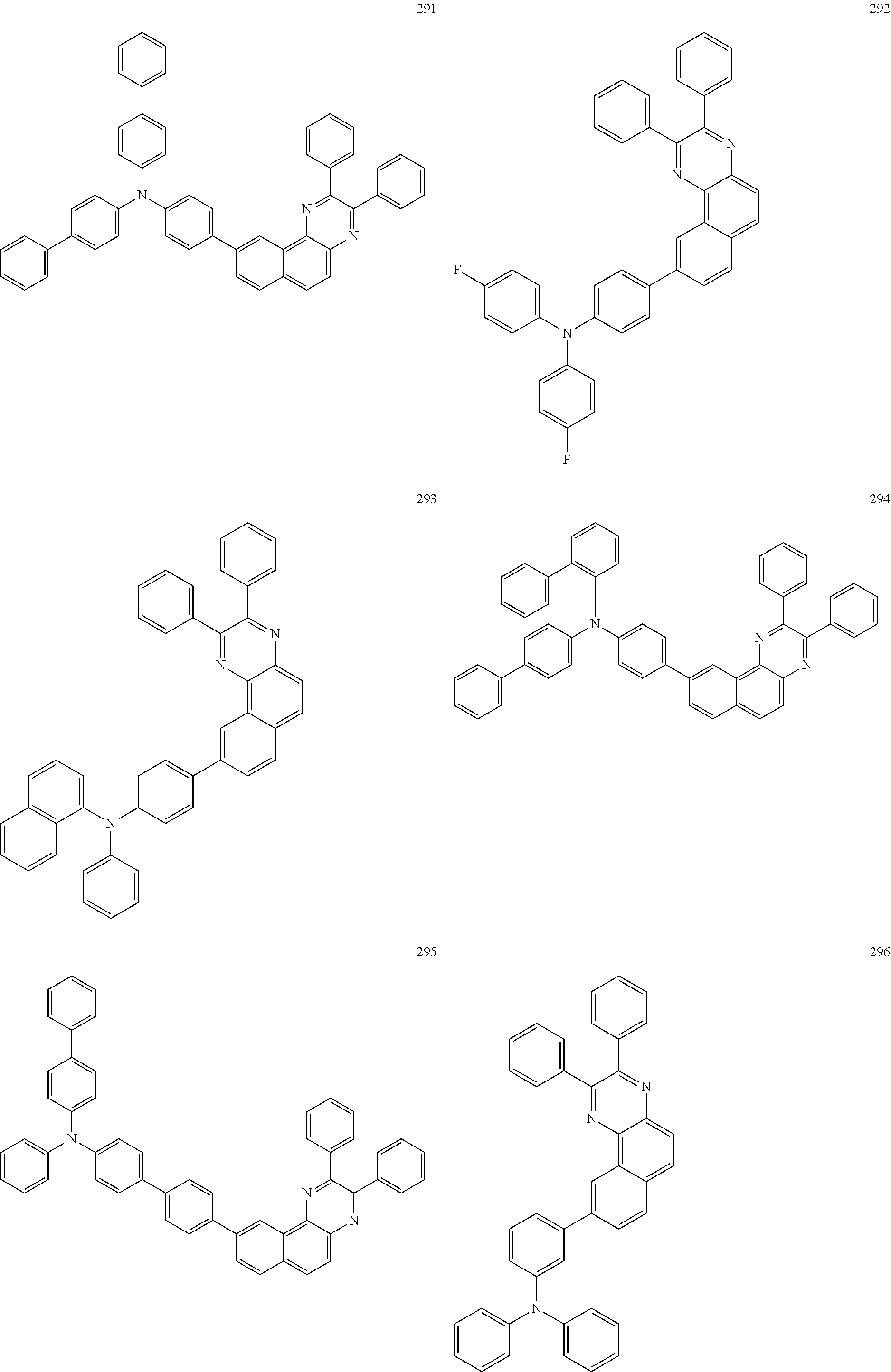

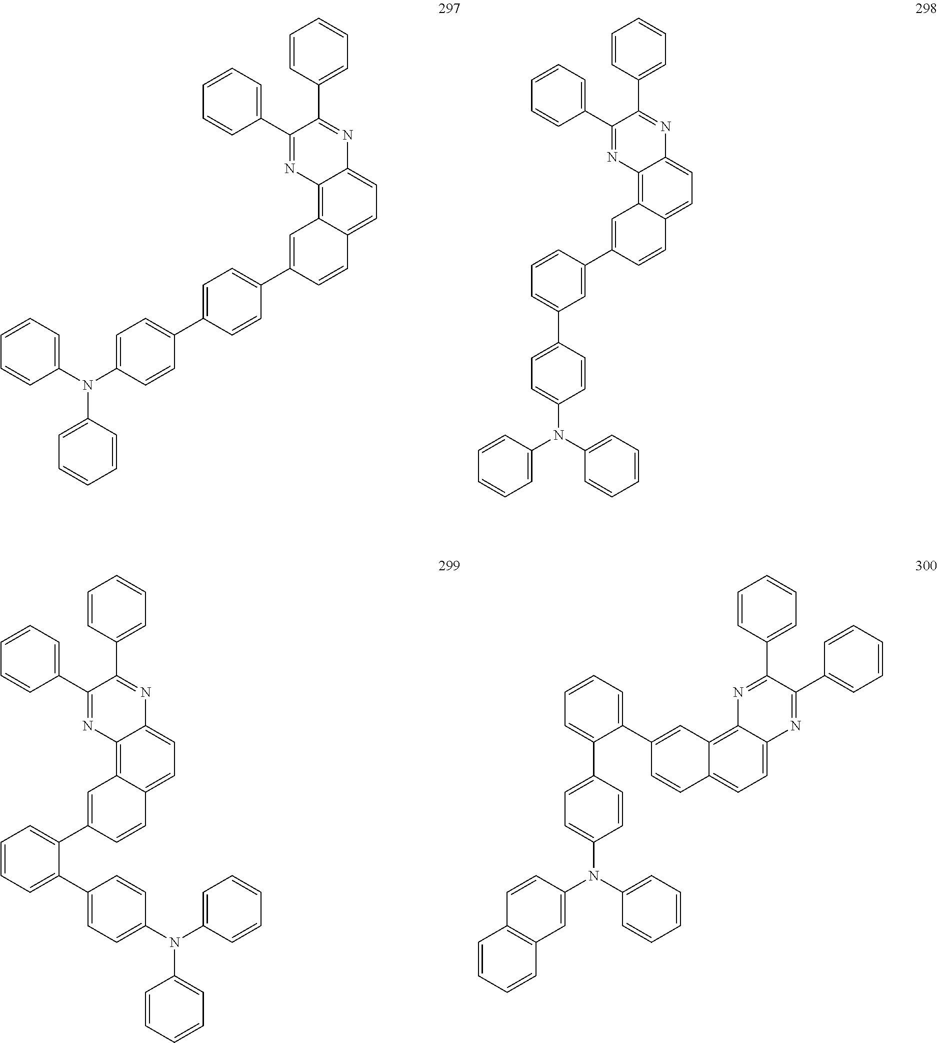 [SMILES: c1ccc(-c2nc3ccc4ccc(-c5ccc(-c6ccc(N(c7ccccc7)c7ccccc7)cc6)cc5)cc4c3nc2-c2ccccc2)cc1 ; c1ccc(-c2nc3ccc4ccc(-c5cccc(-c6ccc(N(c7ccccc7)c7ccccc7)cc6)c5)cc4c3nc2-c2ccccc2)cc1 ; c1ccc(-c2nc3ccc4ccc(-c5ccccc5-c5ccc(N(c6ccccc6)c6ccccc6)cc5)cc4c3nc2-c2ccccc2)cc1 ; c1ccc(-c2nc3ccc4ccc(-c5ccccc5-c5ccc(N(c6ccccc6)c6ccc7ccccc7c6)cc5)cc4c3nc2-c2ccccc2)cc1]

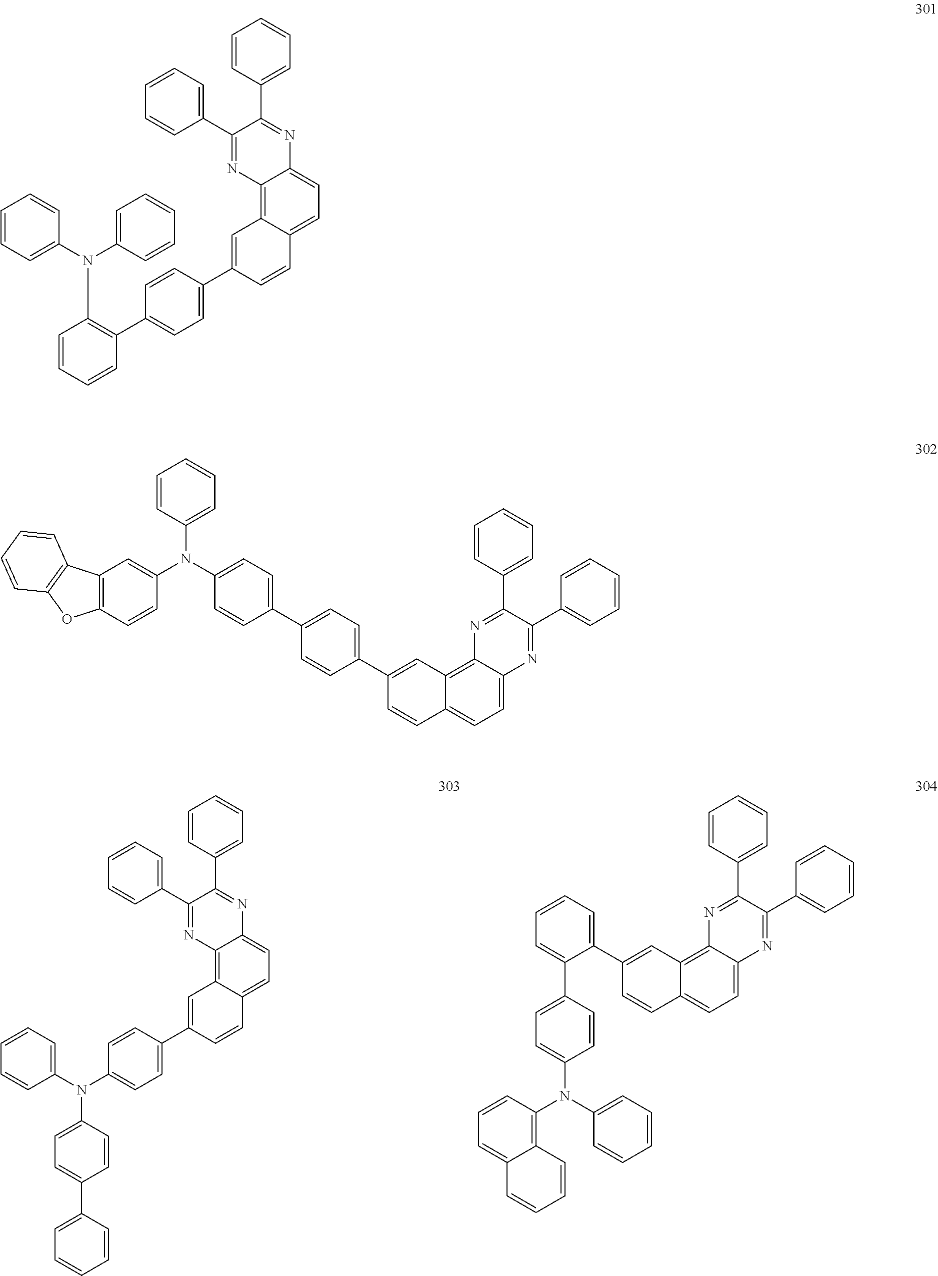 [SMILES: c1ccc(-c2nc3ccc4ccc(-c5ccc(-c6ccccc6N(c6ccccc6)c6ccccc6)cc5)cc4c3nc2-c2ccccc2)cc1 ; c1ccc(-c2nc3ccc4ccc(-c5ccc(-c6ccc(N(c7ccccc7)c7ccc8oc9ccccc9c8c7)cc6)cc5)cc4c3nc2-c2ccccc2)cc1 ; c1ccc(-c2ccc(N(c3ccccc3)c3ccc(-c4ccc5ccc6nc(-c7ccccc7)c(-c7ccccc7)nc6c5c4)cc3)cc2)cc1 ; c1ccc(-c2nc3ccc4ccc(-c5ccccc5-c5ccc(N(c6ccccc6)c6cccc7ccccc67)cc5)cc4c3nc2-c2ccccc2)cc1]

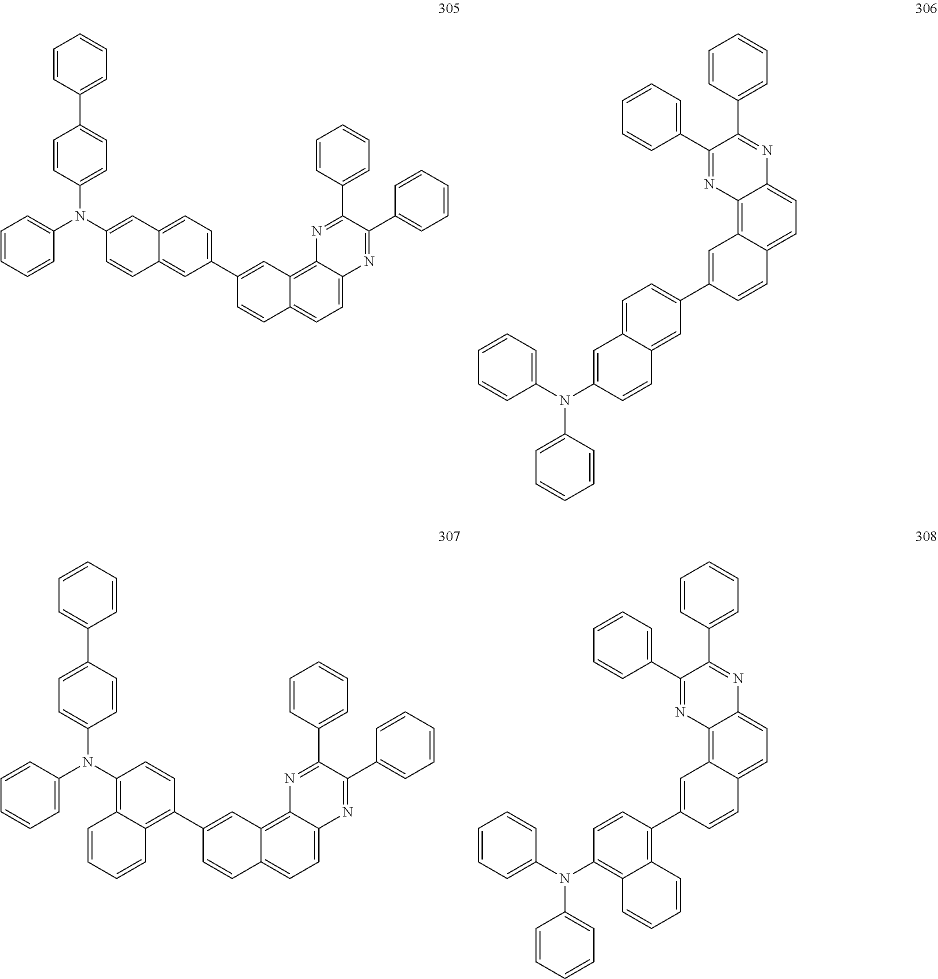 [SMILES: c1ccc(-c2ccc(N(c3ccccc3)c3ccc4cc(-c5ccc6ccc7nc(-c8ccccc8)c(-c8ccccc8)nc7c6c5)ccc4c3)cc2)cc1 ; c1ccc(-c2nc3ccc4ccc(-c5ccc6cc(N(c7ccccc7)c7ccccc7)ccc6c5)cc4c3nc2-c2ccccc2)cc1 ; c1ccc(-c2ccc(N(c3ccccc3)c3ccc(-c4ccc5ccc6nc(-c7ccccc7)c(-c7ccccc7)nc6c5c4)c4ccccc34)cc2)cc1 ; c1ccc(-c2nc3ccc4ccc(-c5ccc(N(c6ccccc6)c6ccccc6)c6ccccc56)cc4c3nc2-c2ccccc2)cc1]

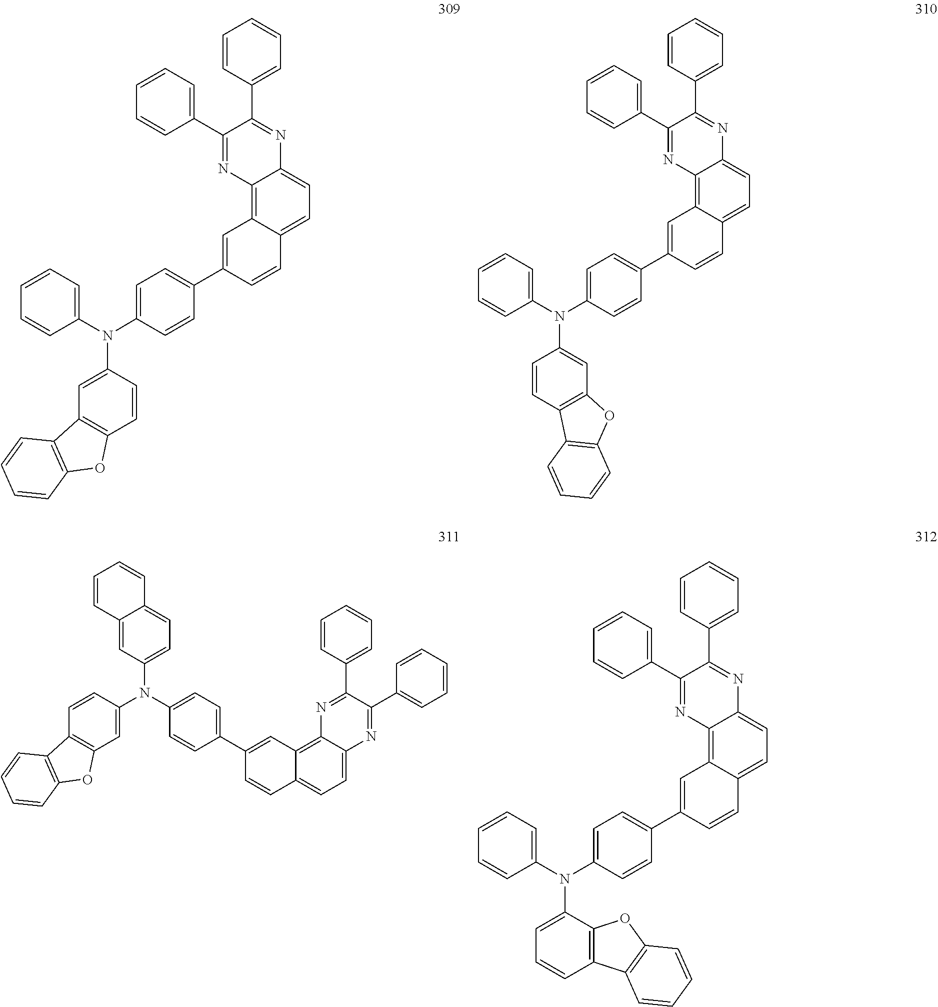 [SMILES: c1ccc(-c2nc3ccc4ccc(-c5ccc(N(c6ccccc6)c6ccc7oc8ccccc8c7c6)cc5)cc4c3nc2-c2ccccc2)cc1 ; c1ccc(-c2nc3ccc4ccc(-c5ccc(N(c6ccccc6)c6ccc7c(c6)oc6ccccc67)cc5)cc4c3nc2-c2ccccc2)cc1 ; c1ccc(-c2nc3ccc4ccc(-c5ccc(N(c6ccc7ccccc7c6)c6ccc7c(c6)oc6ccccc67)cc5)cc4c3nc2-c2ccccc2)cc1 ; c1ccc(-c2nc3ccc4ccc(-c5ccc(N(c6ccccc6)c6cccc7c6oc6ccccc67)cc5)cc4c3nc2-c2ccccc2)cc1]

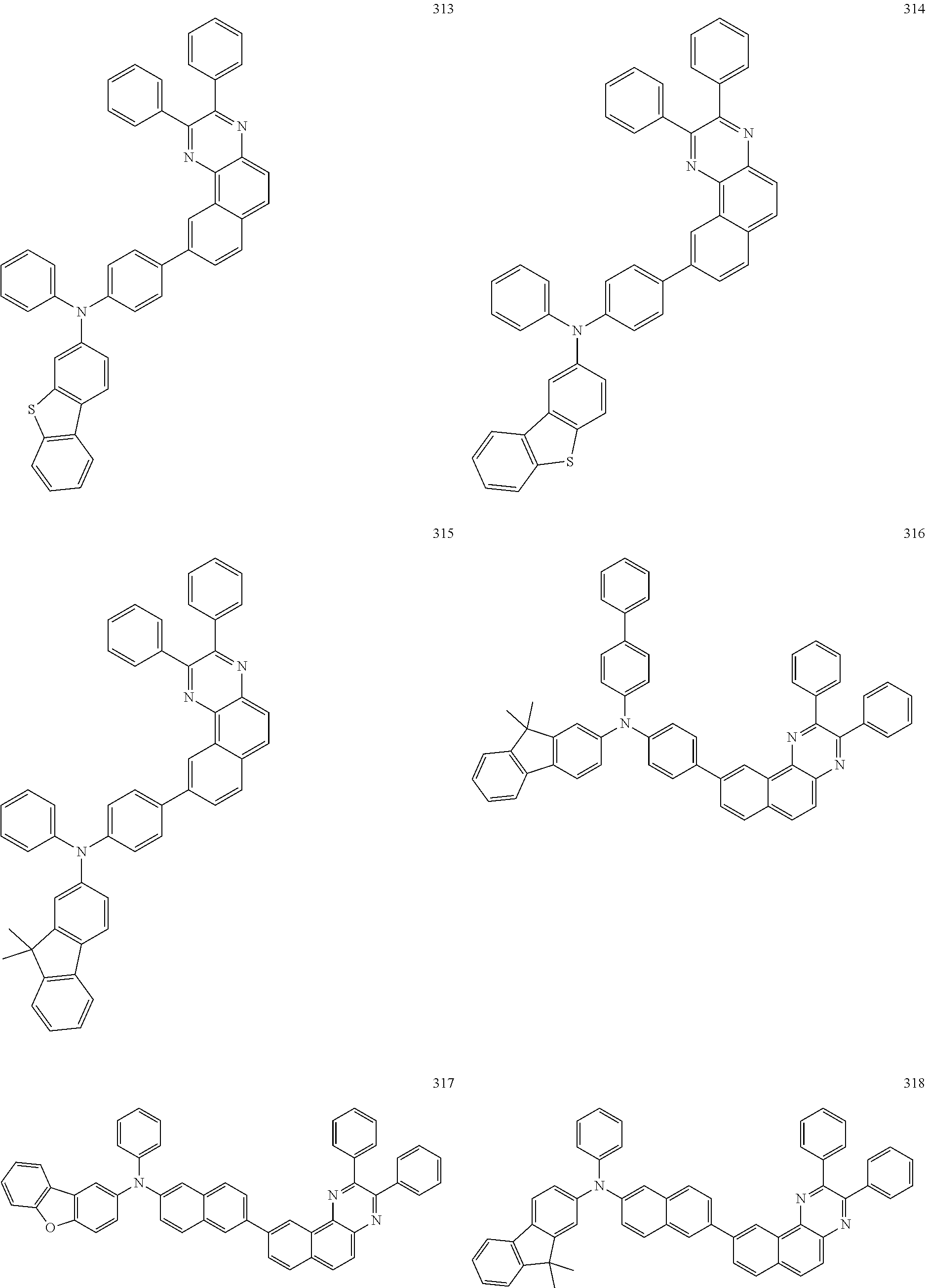

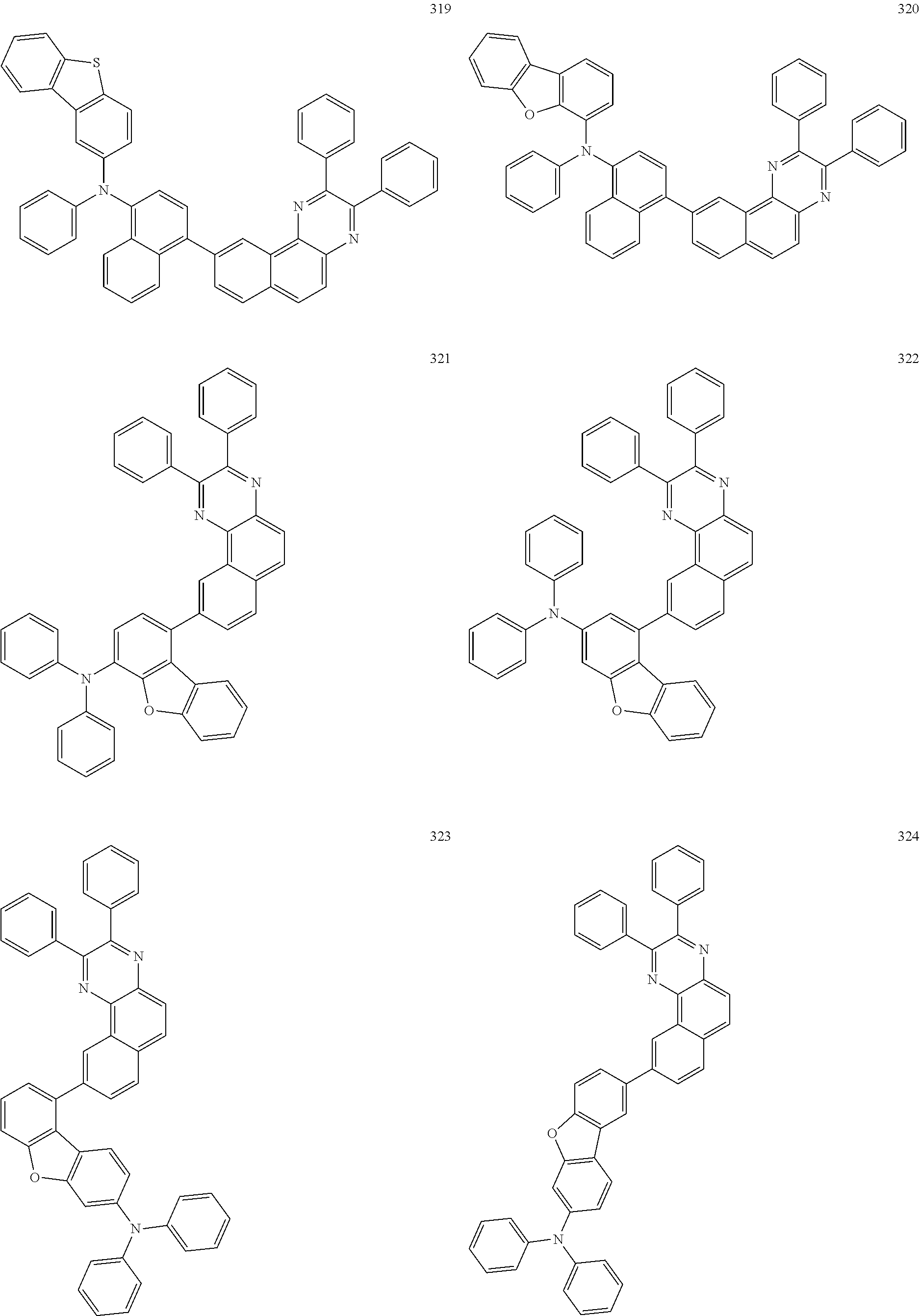

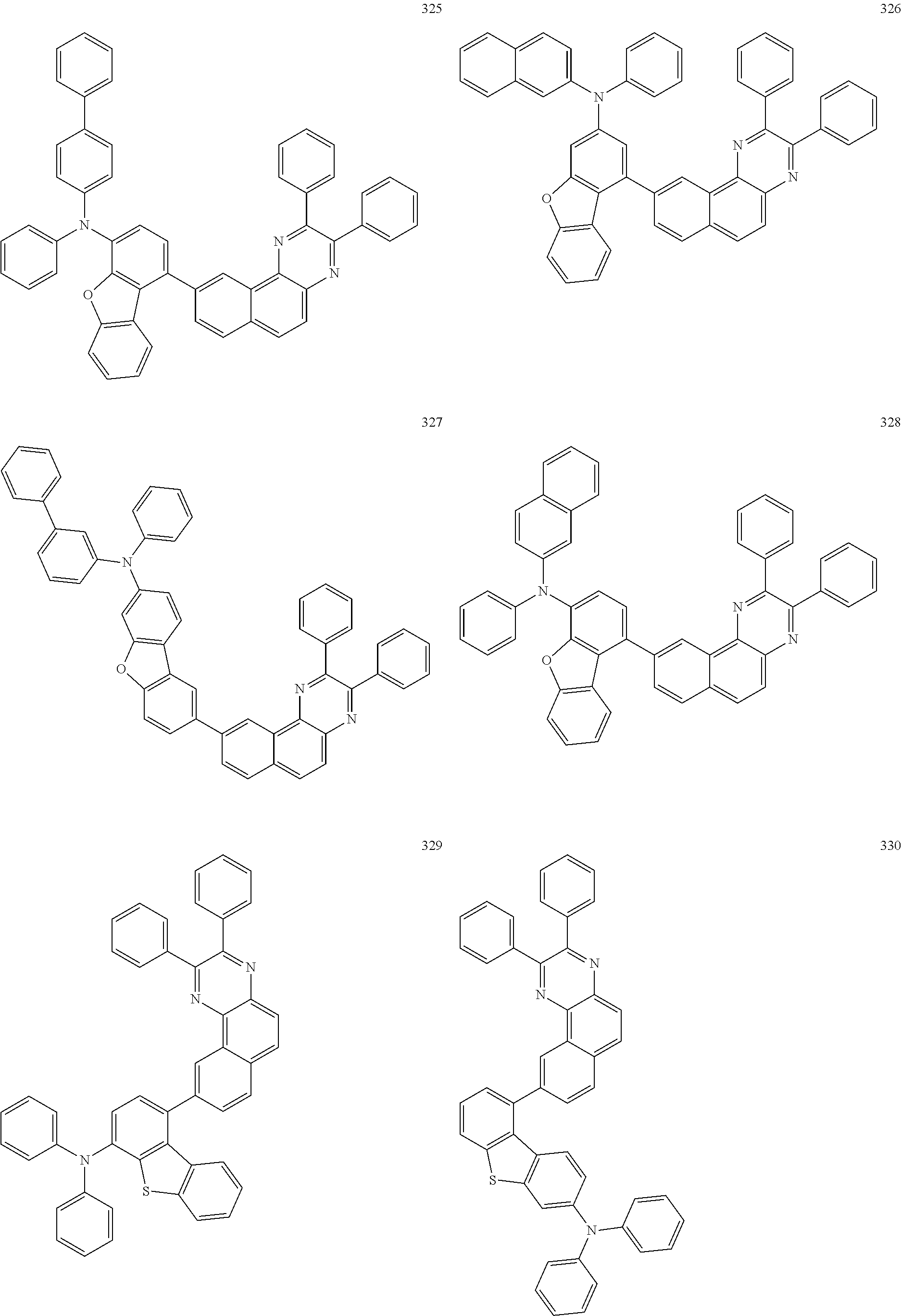

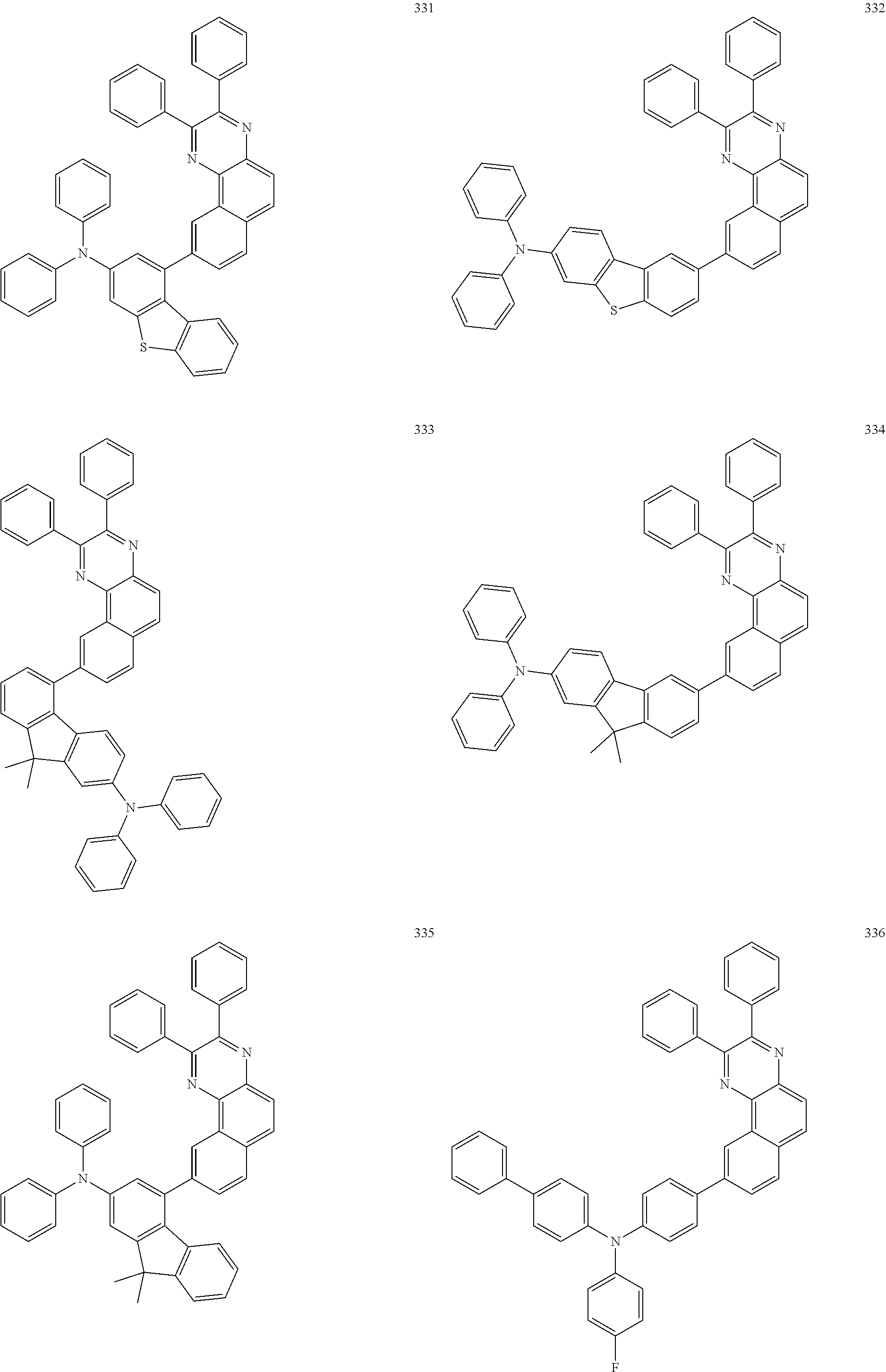

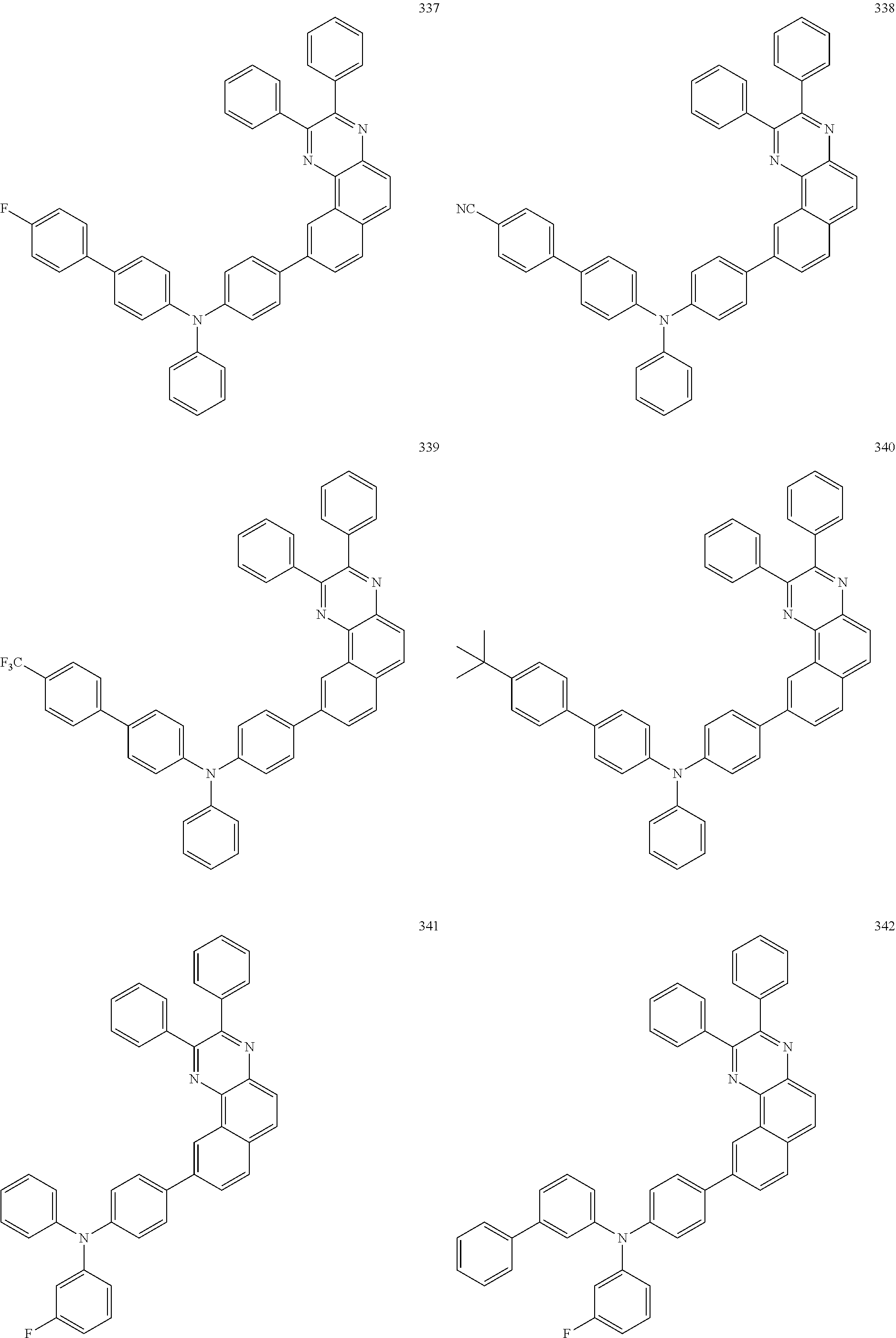

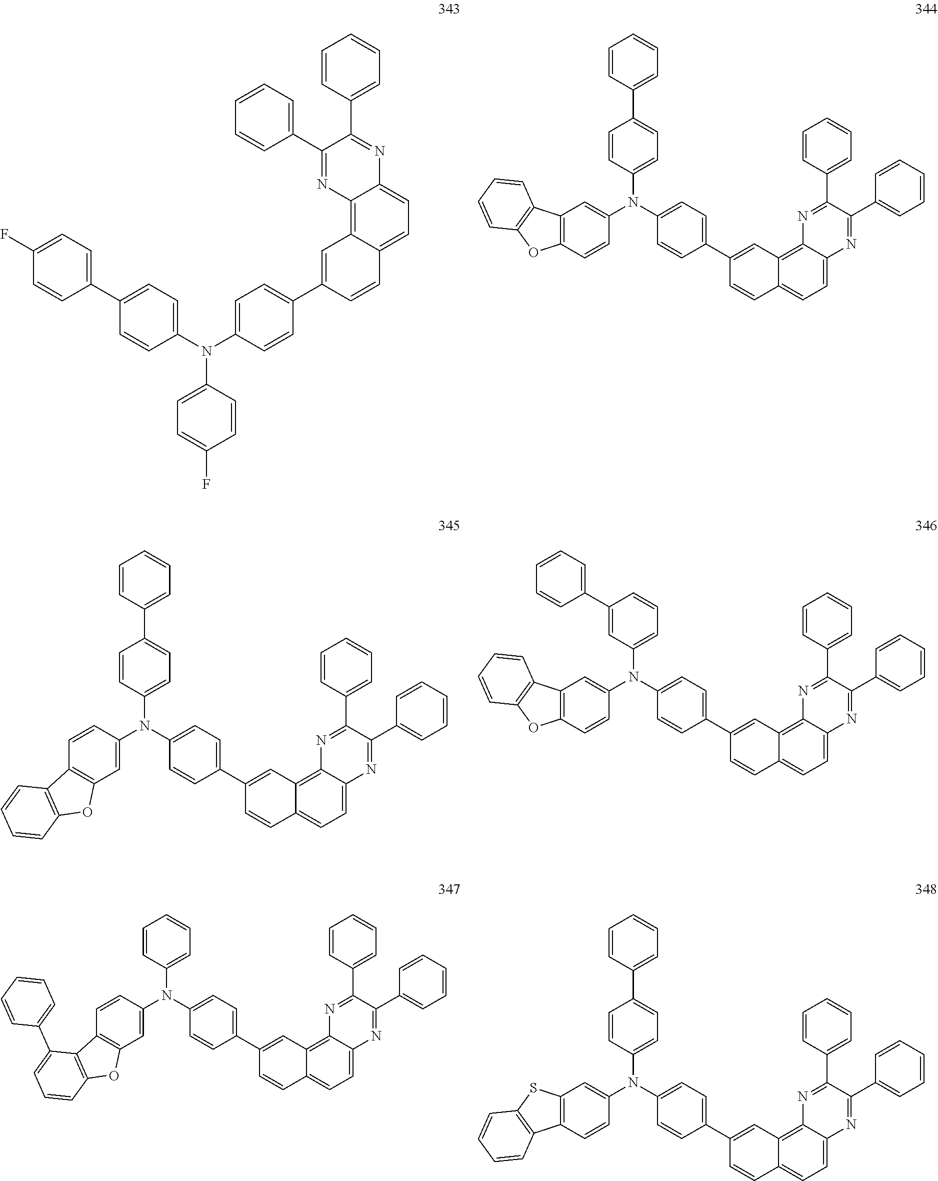

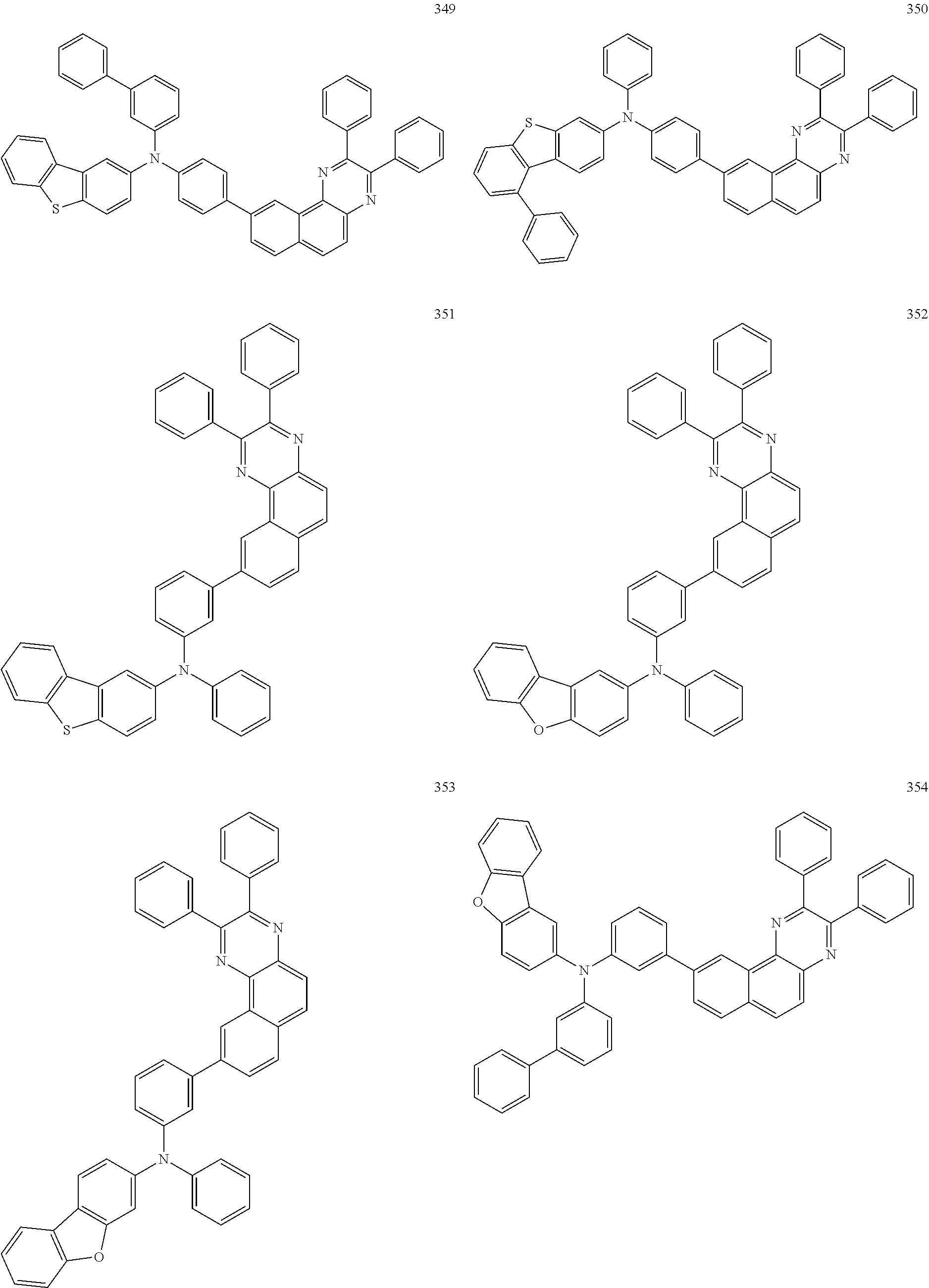

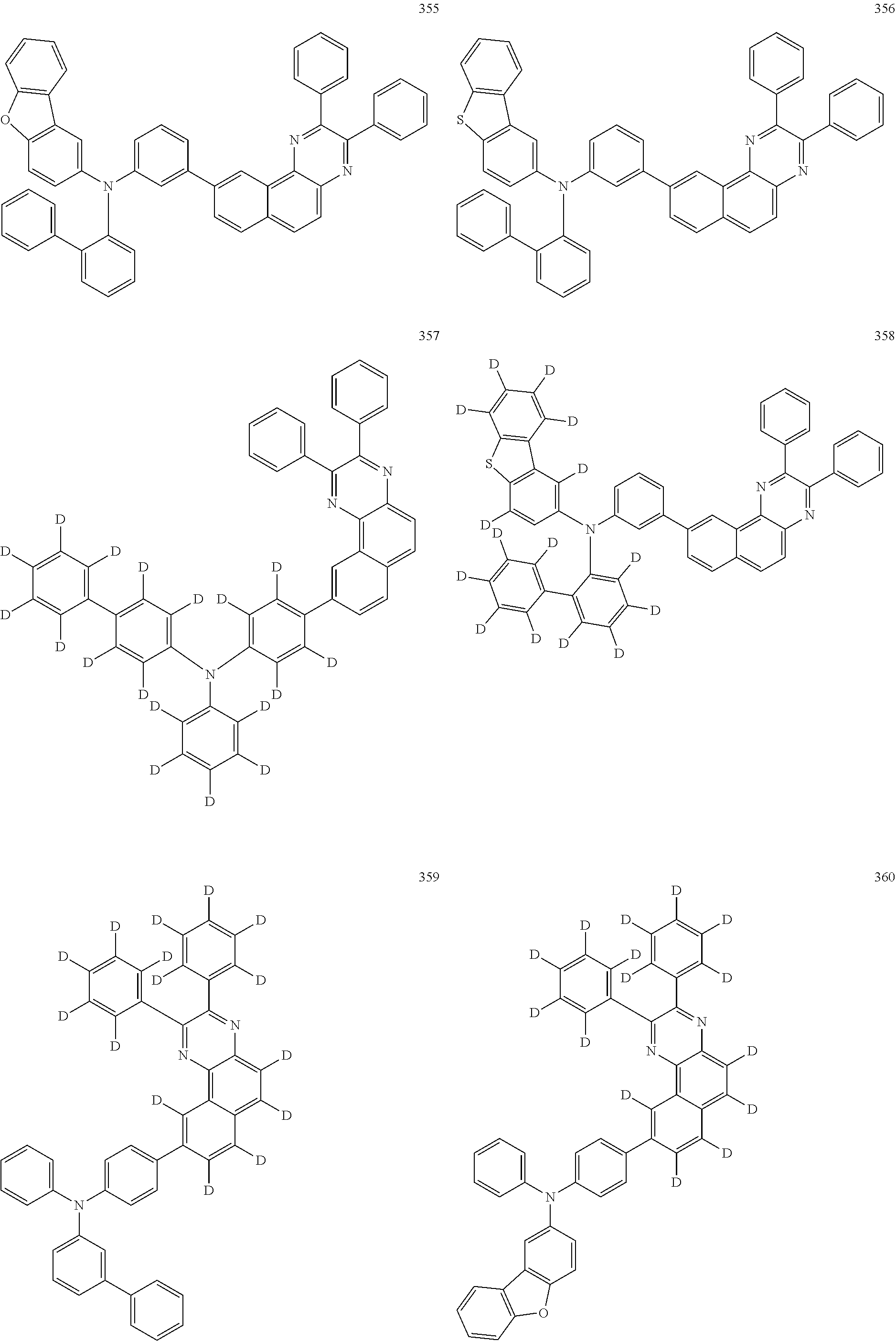

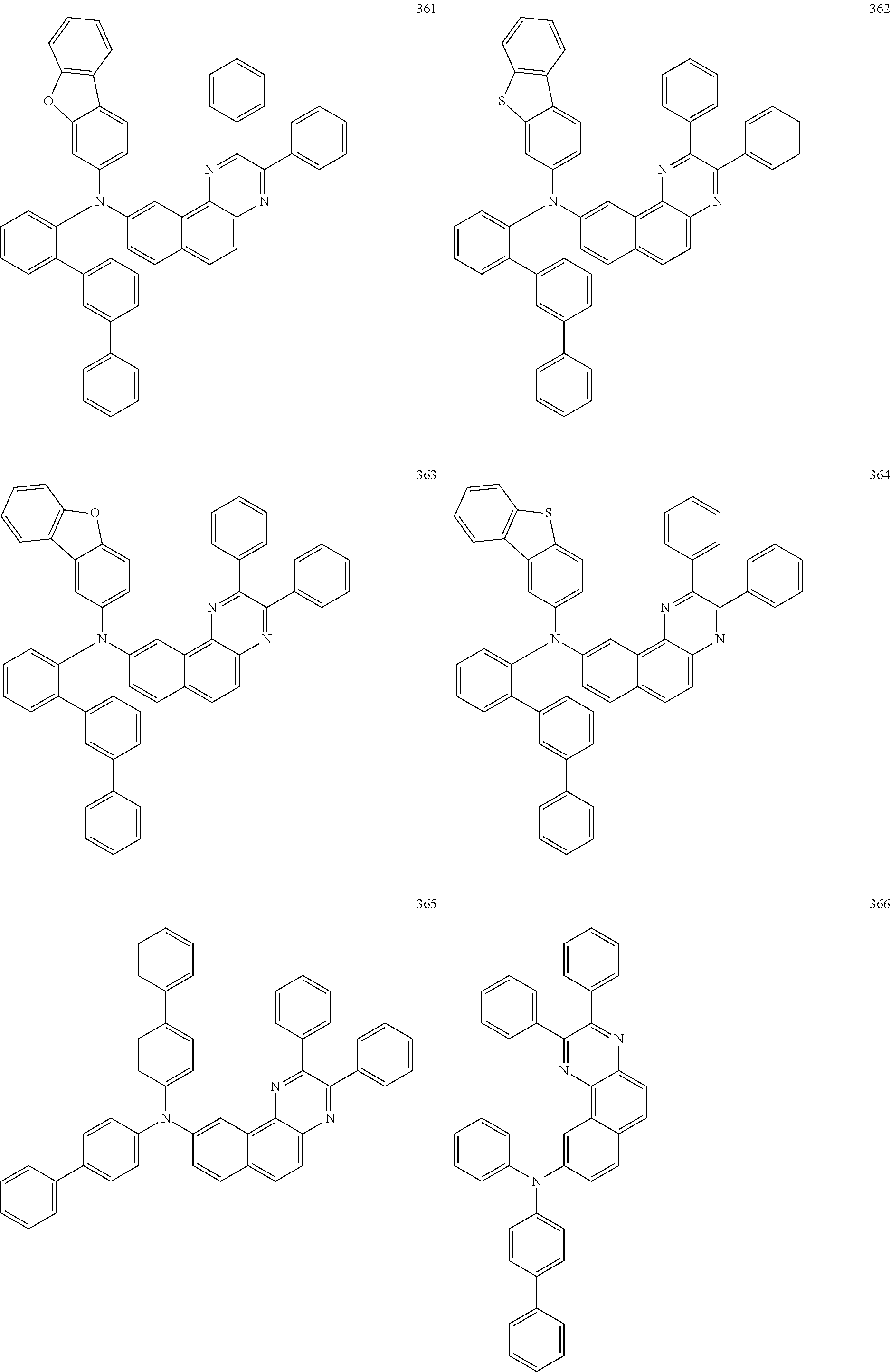

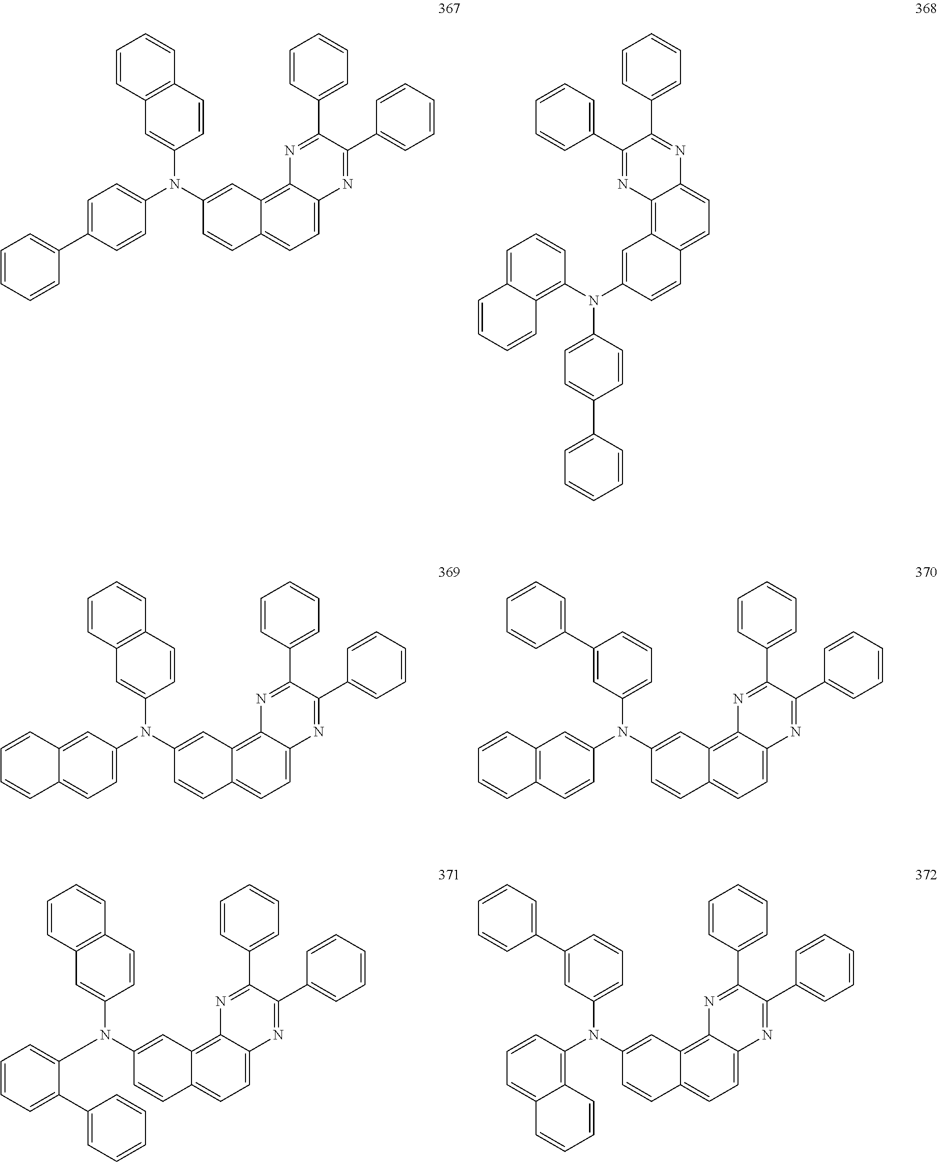 [SMILES: c1ccc(-c2ccc(N(c3ccc4ccccc4c3)c3ccc4ccc5nc(-c6ccccc6)c(-c6ccccc6)nc5c4c3)cc2)cc1 ; c1ccc(-c2ccc(N(c3ccc4ccc5nc(-c6ccccc6)c(-c6ccccc6)nc5c4c3)c3cccc4ccccc34)cc2)cc1 ; c1ccc(-c2nc3ccc4ccc(N(c5ccc6ccccc6c5)c5ccc6ccccc6c5)cc4c3nc2-c2ccccc2)cc1 ; c1ccc(-c2cccc(N(c3ccc4ccccc4c3)c3ccc4ccc5nc(-c6ccccc6)c(-c6ccccc6)nc5c4c3)c2)cc1 ; c1ccc(-c2ccccc2N(c2ccc3ccccc3c2)c2ccc3ccc4nc(-c5ccccc5)c(-c5ccccc5)nc4c3c2)cc1 ; c1ccc(-c2cccc(N(c3ccc4ccc5nc(-c6ccccc6)c(-c6ccccc6)nc5c4c3)c3cccc4ccccc34)c2)cc1]

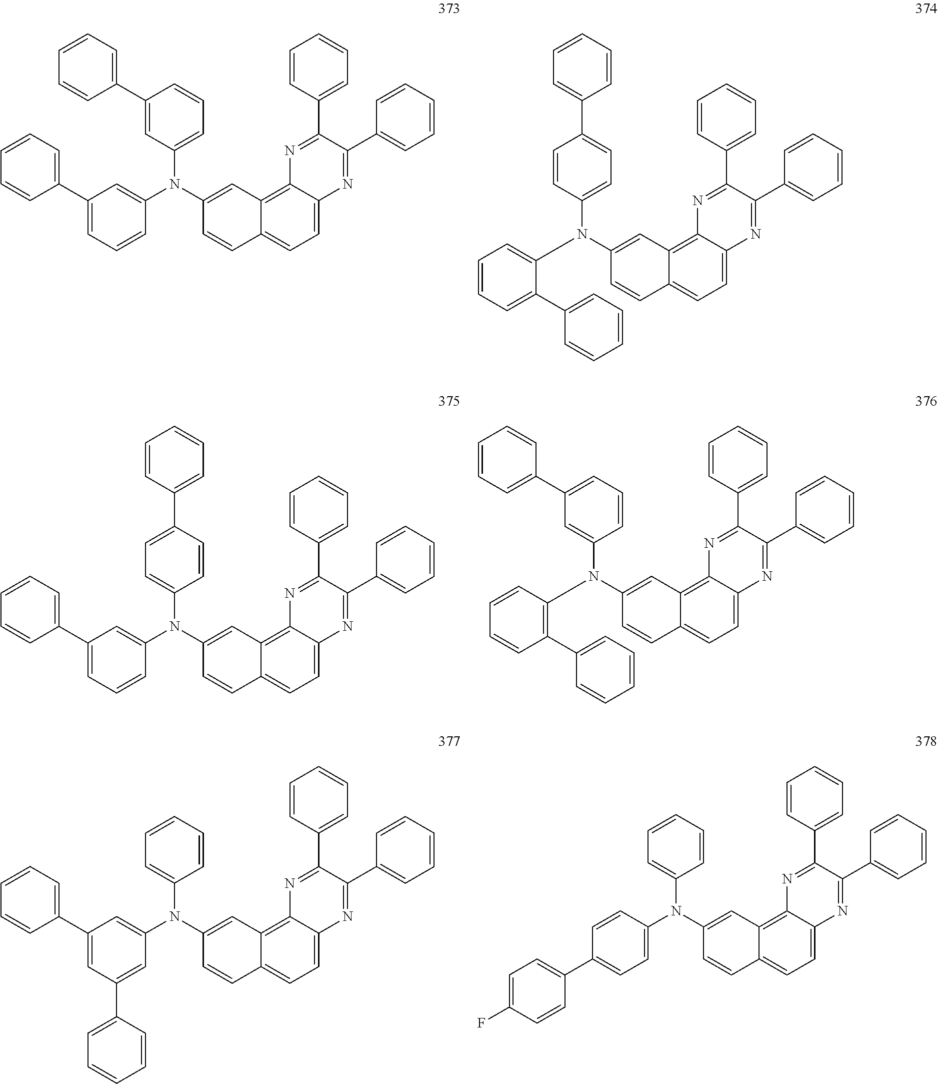

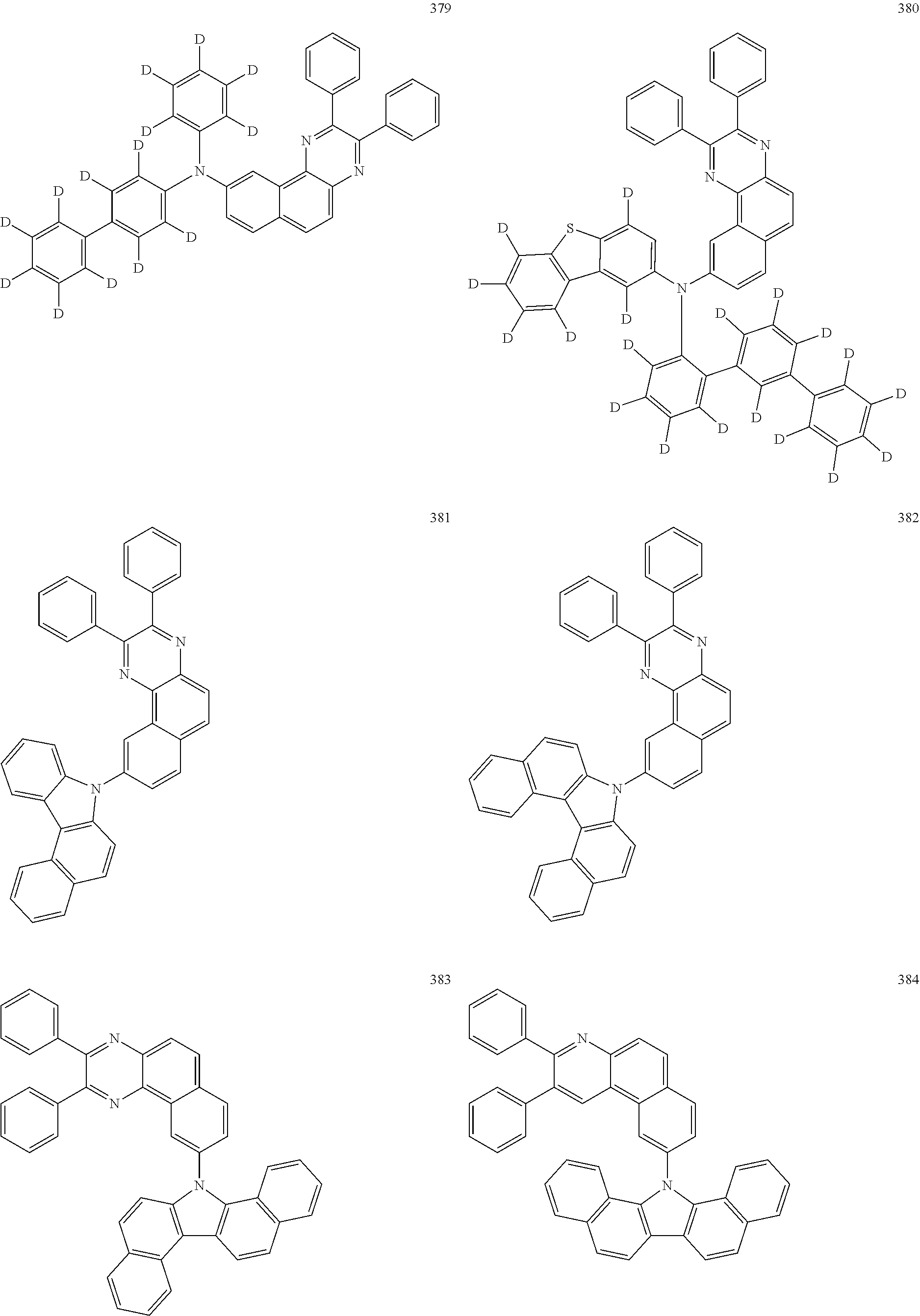

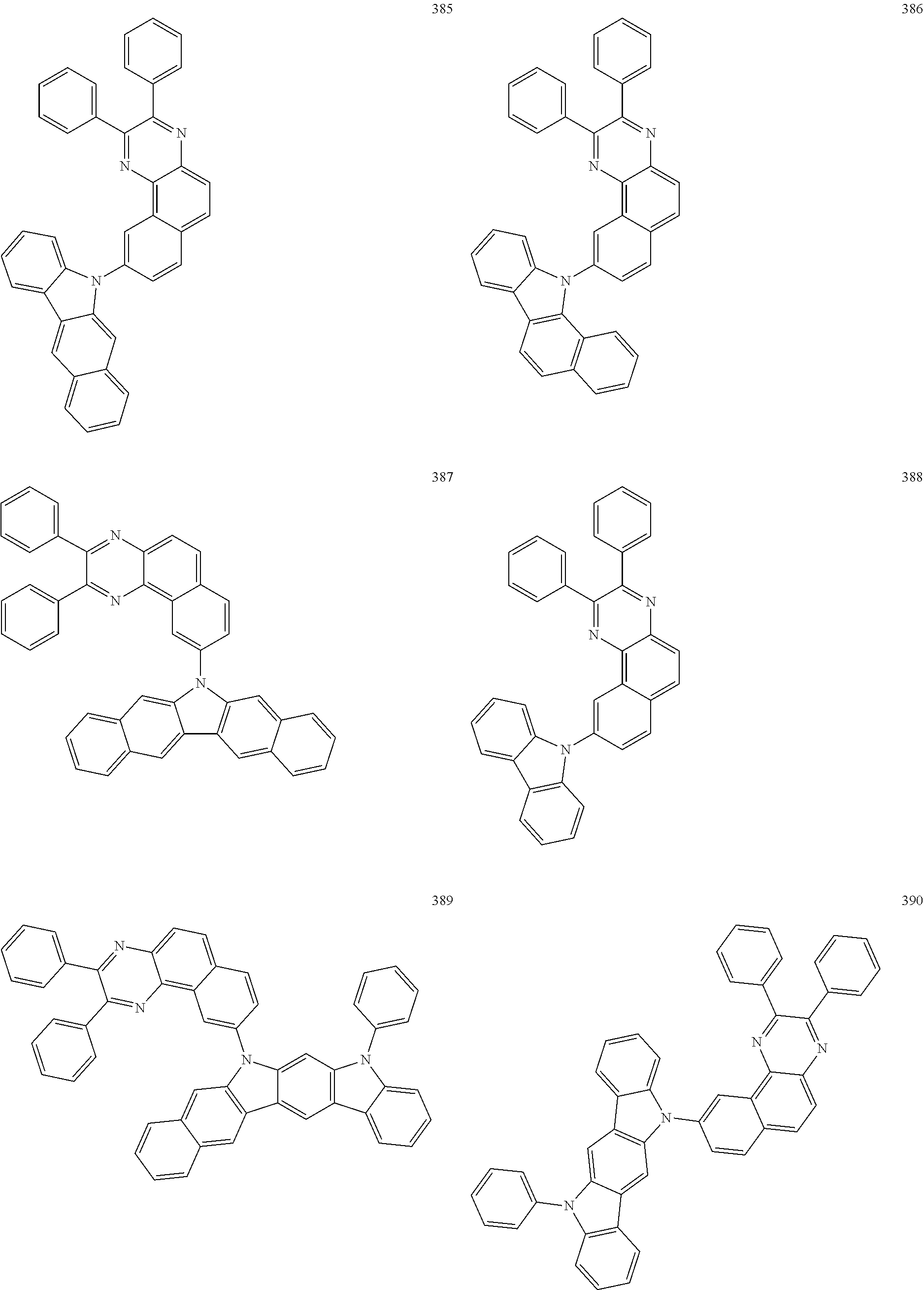 [SMILES: c1ccc(-c2nc3ccc4ccc(-n5c6ccccc6c6cc7ccccc7cc65)cc4c3nc2-c2ccccc2)cc1 ; c1ccc(-c2nc3ccc4ccc(-n5c6ccccc6c6ccc7ccccc7c65)cc4c3nc2-c2ccccc2)cc1 ; c1ccc(-c2nc3ccc4ccc(-n5c6cc7ccccc7cc6c6cc7ccccc7cc65)cc4c3nc2-c2ccccc2)cc1 ; c1ccc(-c2nc3ccc4ccc(-n5c6ccccc6c6ccccc65)cc4c3nc2-c2ccccc2)cc1 ; c1ccc(-c2nc3ccc4ccc(-n5c6cc7ccccc7cc6c6cc7c8ccccc8n(-c8ccccc8)c7cc65)cc4c3nc2-c2ccccc2)cc1 ; c1ccc(-c2nc3ccc4ccc(-n5c6ccccc6c6cc7c(cc65)c5ccccc5n7-c5ccccc5)cc4c3nc2-c2ccccc2)cc1]

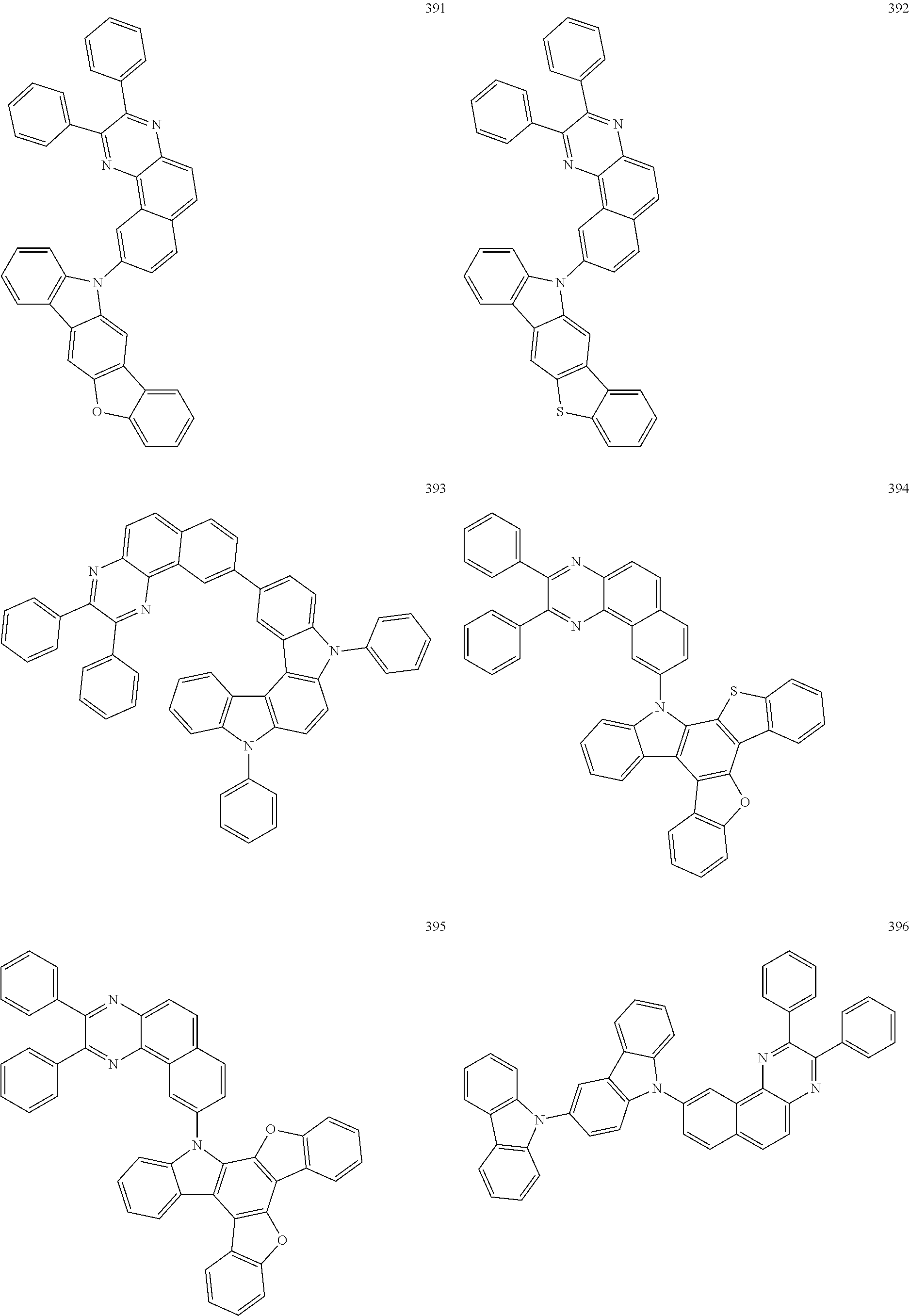

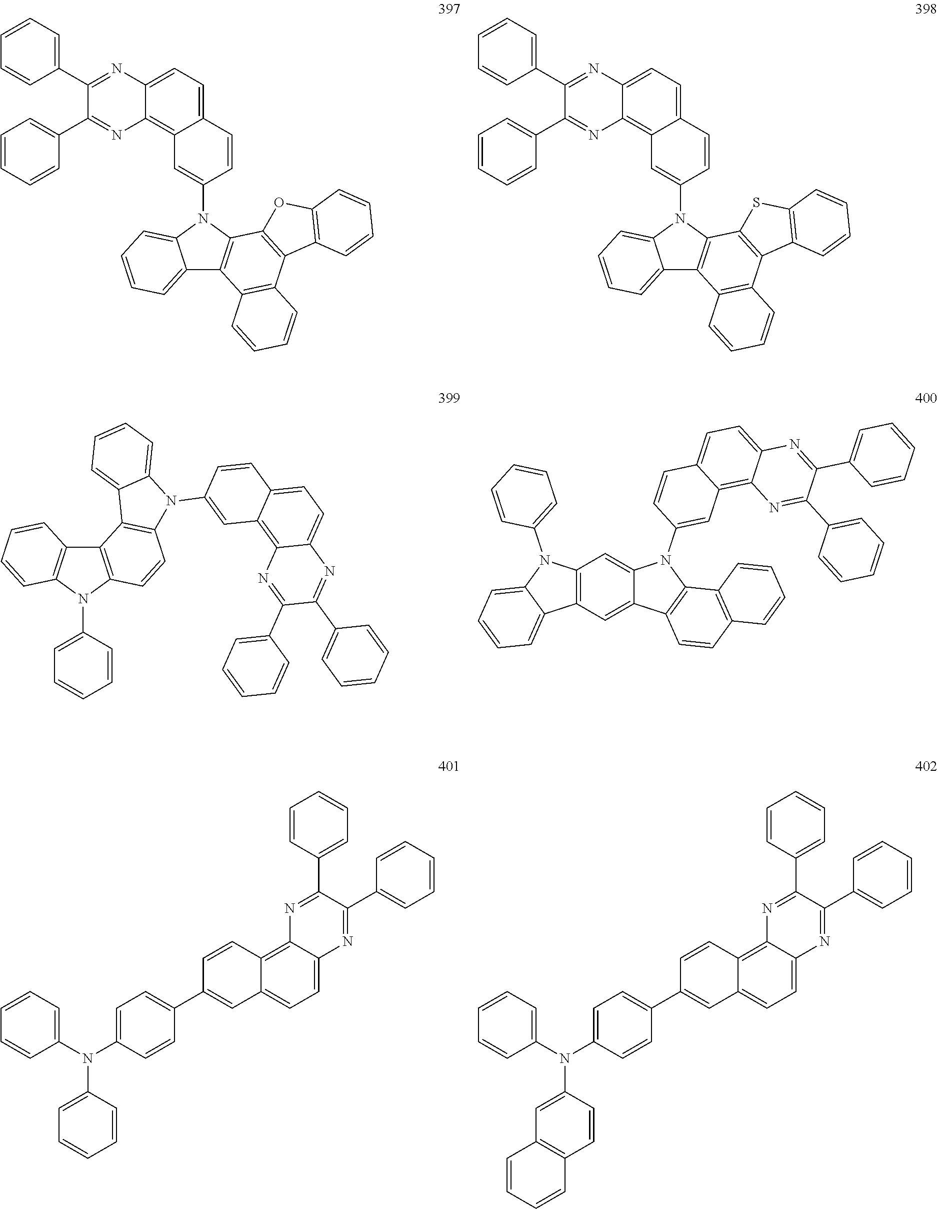 [SMILES: c1ccc(-c2nc3ccc4ccc(-n5c6ccccc6c6c7ccccc7c7c8ccccc8oc7c65)cc4c3nc2-c2ccccc2)cc1 ; c1ccc(-c2nc3ccc4ccc(-n5c6ccccc6c6c7ccccc7c7c8ccccc8sc7c65)cc4c3nc2-c2ccccc2)cc1 ; c1ccc(-c2nc3ccc4ccc(-n5c6ccccc6c6c7c8ccccc8n(-c8ccccc8)c7ccc65)cc4c3nc2-c2ccccc2)cc1 ; c1ccc(-c2nc3ccc4ccc(-n5c6cc7c(cc6c6ccc8ccccc8c65)c5ccccc5n7-c5ccccc5)cc4c3nc2-c2ccccc2)cc1 ; c1ccc(-c2nc3ccc4cc(-c5ccc(N(c6ccccc6)c6ccccc6)cc5)ccc4c3nc2-c2ccccc2)cc1 ; c1ccc(-c2nc3ccc4cc(-c5ccc(N(c6ccccc6)c6ccc7ccccc7c6)cc5)ccc4c3nc2-c2ccccc2)cc1]

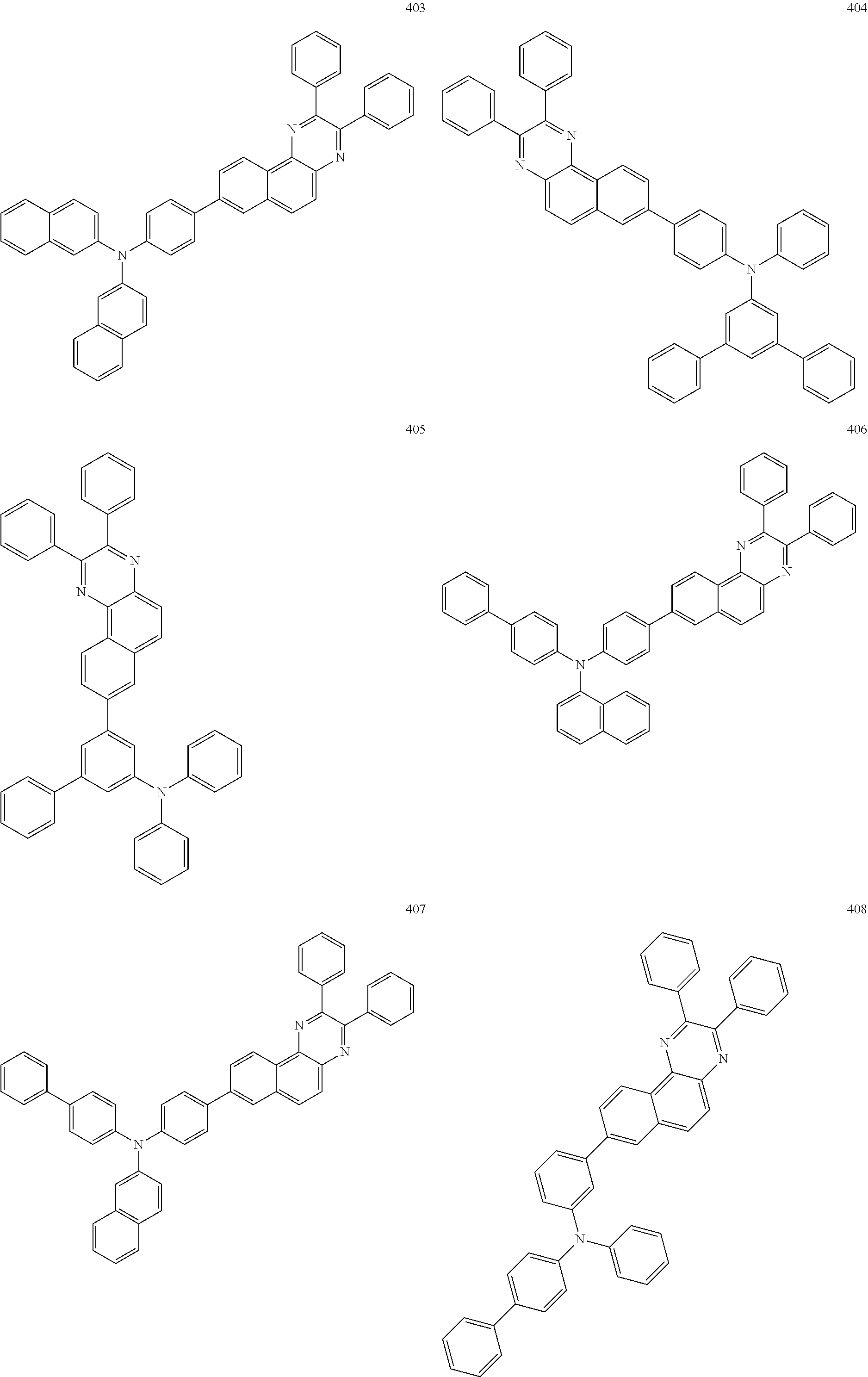

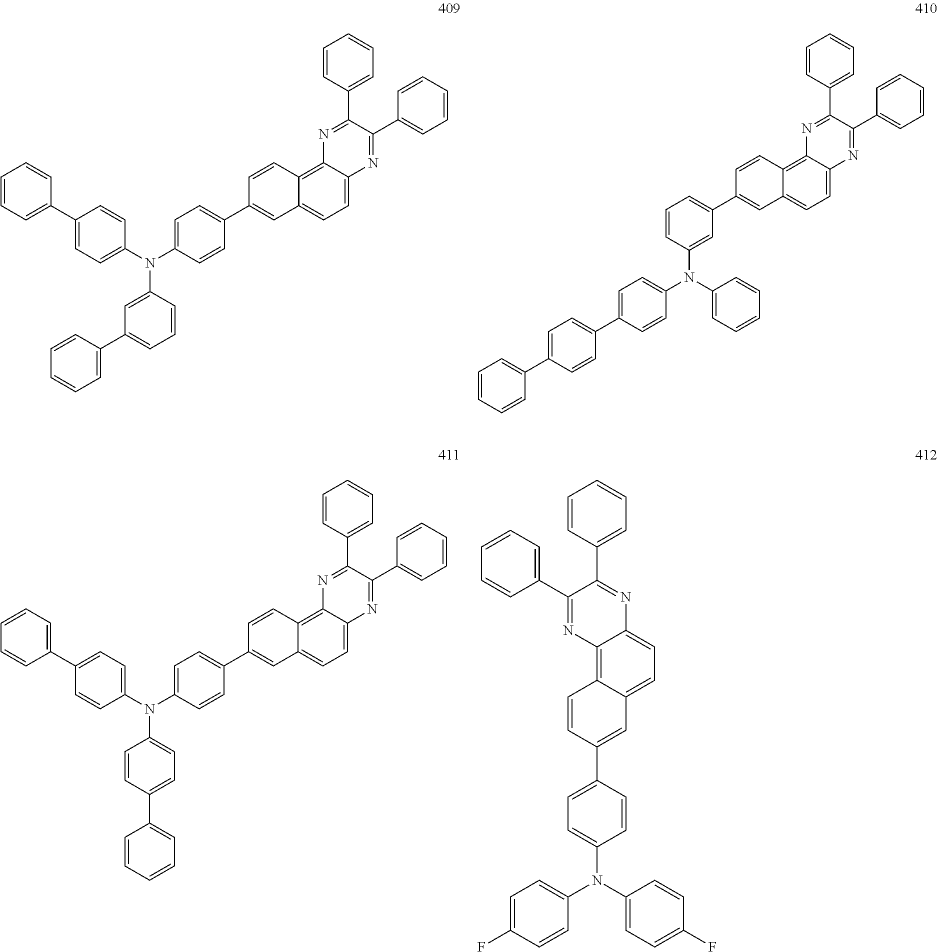

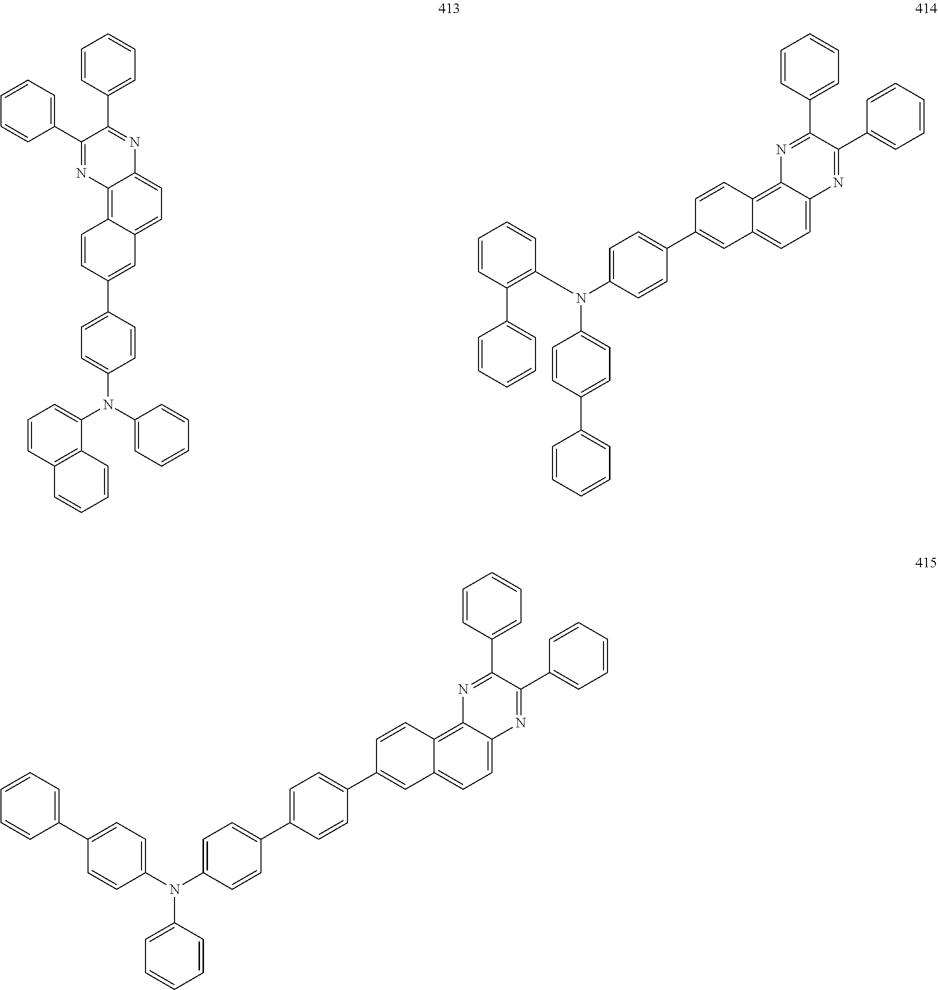 [SMILES: c1ccc(-c2nc3ccc4cc(-c5ccc(N(c6ccccc6)c6cccc7ccccc67)cc5)ccc4c3nc2-c2ccccc2)cc1 ; c1ccc(-c2ccc(N(c3ccc(-c4ccc5c(ccc6nc(-c7ccccc7)c(-c7ccccc7)nc65)c4)cc3)c3ccccc3-c3ccccc3)cc2)cc1 ; c1ccc(-c2ccc(N(c3ccccc3)c3ccc(-c4ccc(-c5ccc6c(ccc7nc(-c8ccccc8)c(-c8ccccc8)nc76)c5)cc4)cc3)cc2)cc1]

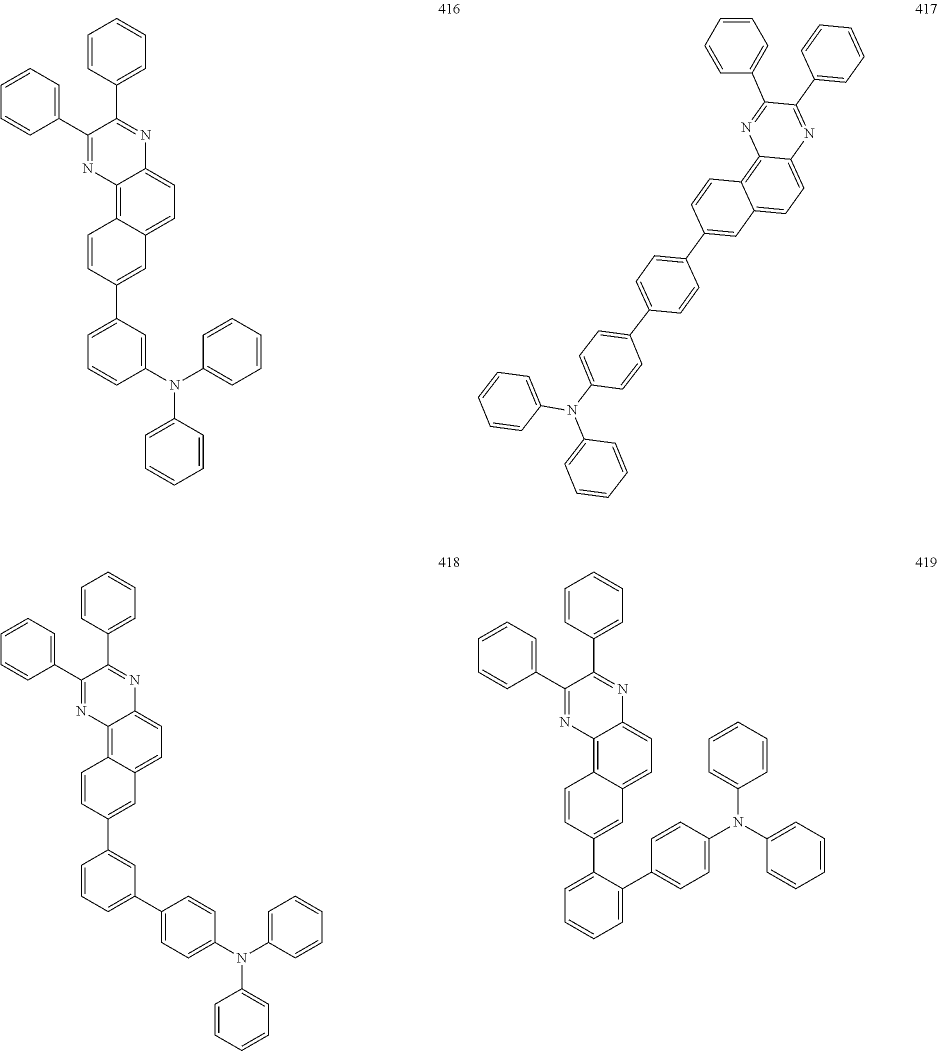 [SMILES: c1ccc(-c2nc3ccc4cc(-c5cccc(N(c6ccccc6)c6ccccc6)c5)ccc4c3nc2-c2ccccc2)cc1 ; c1ccc(-c2nc3ccc4cc(-c5ccc(-c6ccc(N(c7ccccc7)c7ccccc7)cc6)cc5)ccc4c3nc2-c2ccccc2)cc1 ; c1ccc(-c2nc3ccc4cc(-c5cccc(-c6ccc(N(c7ccccc7)c7ccccc7)cc6)c5)ccc4c3nc2-c2ccccc2)cc1 ; c1ccc(-c2nc3ccc4cc(-c5ccccc5-c5ccc(N(c6ccccc6)c6ccccc6)cc5)ccc4c3nc2-c2ccccc2)cc1]

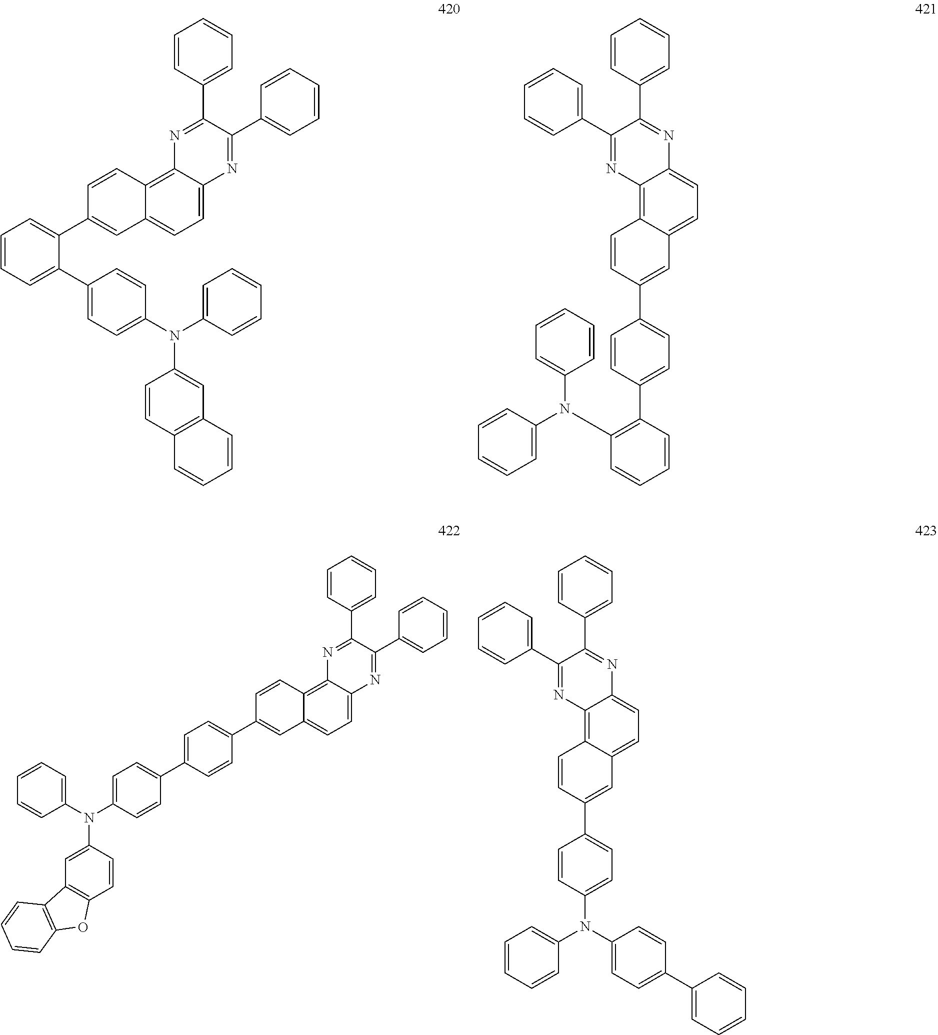

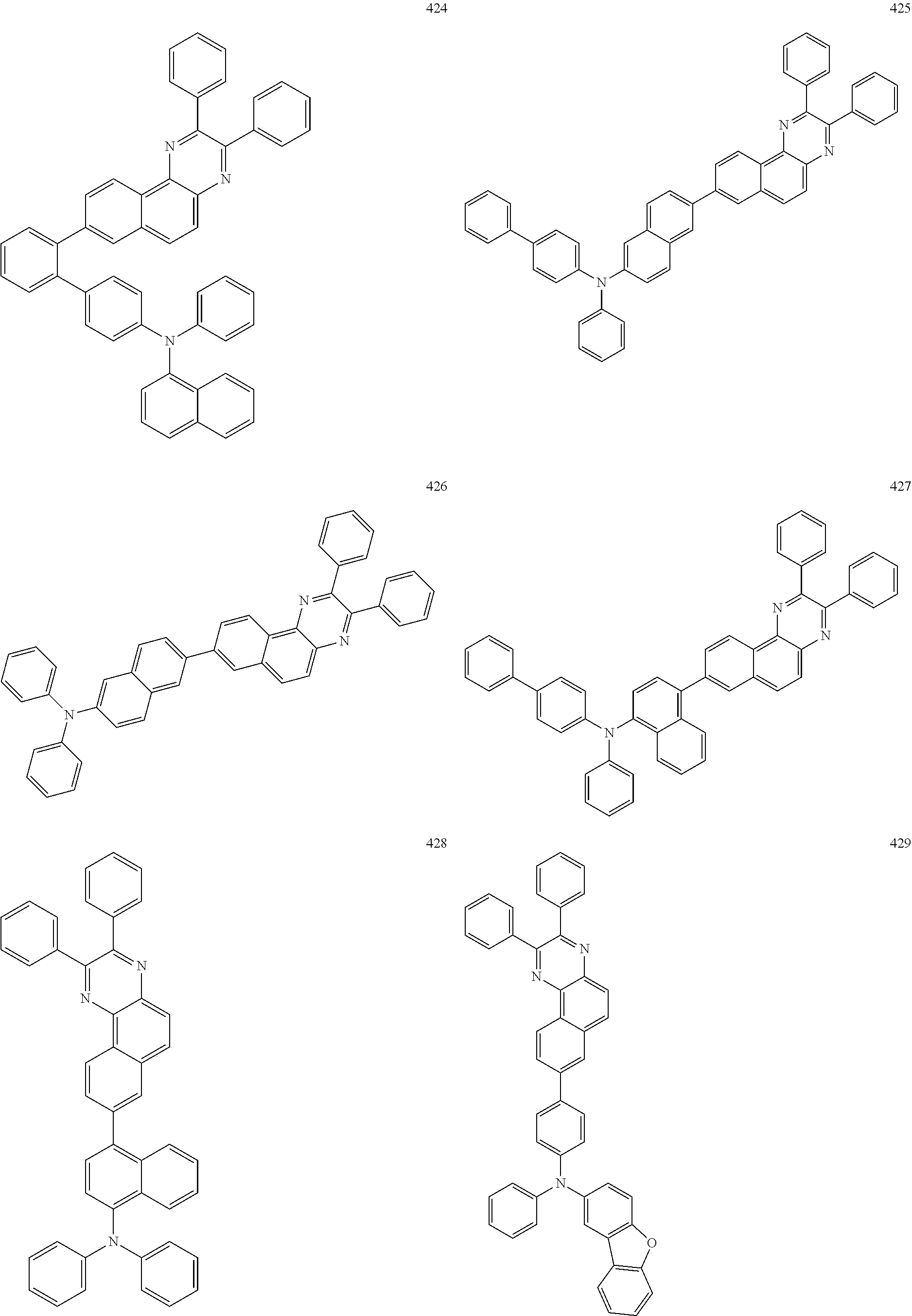

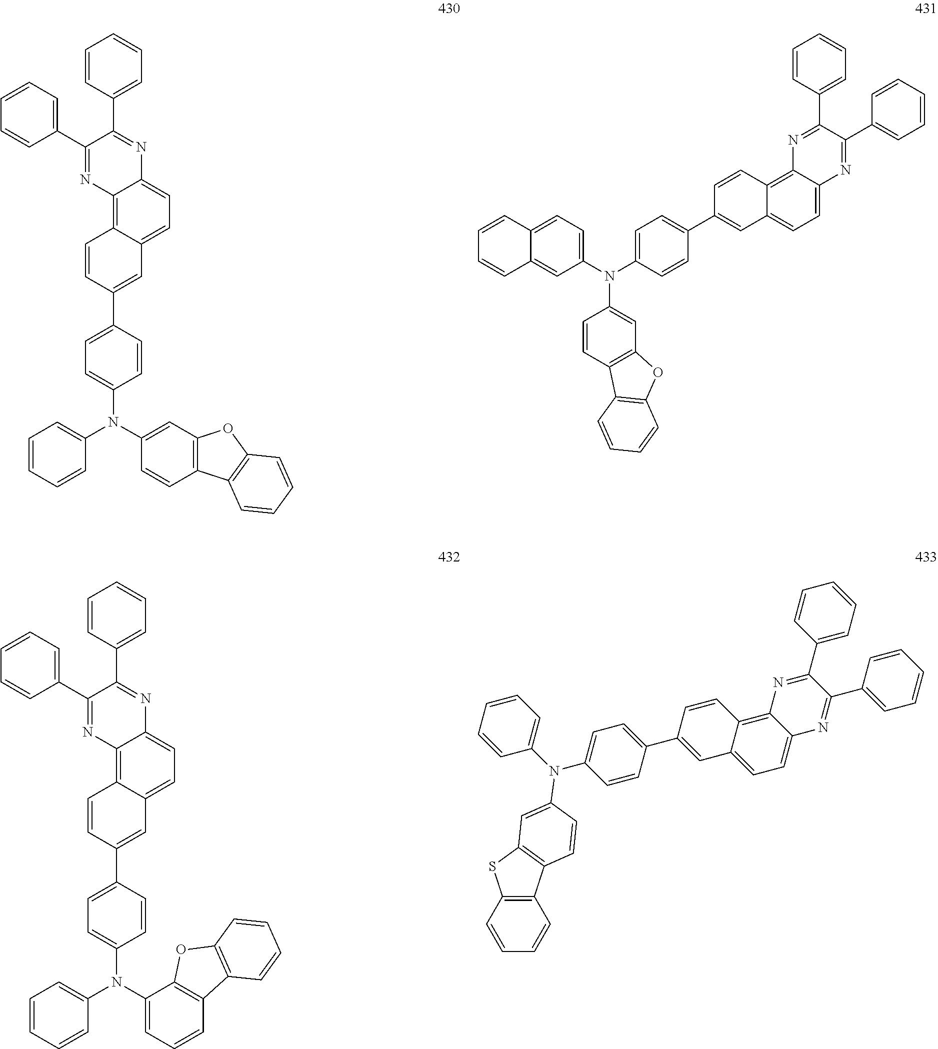 [SMILES: c1ccc(-c2nc3ccc4cc(-c5ccc(N(c6ccccc6)c6ccc7c(c6)oc6ccccc67)cc5)ccc4c3nc2-c2ccccc2)cc1 ; c1ccc(-c2nc3ccc4cc(-c5ccc(N(c6ccc7ccccc7c6)c6ccc7c(c6)oc6ccccc67)cc5)ccc4c3nc2-c2ccccc2)cc1 ; c1ccc(-c2nc3ccc4cc(-c5ccc(N(c6ccccc6)c6cccc7c6oc6ccccc67)cc5)ccc4c3nc2-c2ccccc2)cc1 ; c1ccc(-c2nc3ccc4cc(-c5ccc(N(c6ccccc6)c6ccc7c(c6)sc6ccccc67)cc5)ccc4c3nc2-c2ccccc2)cc1]

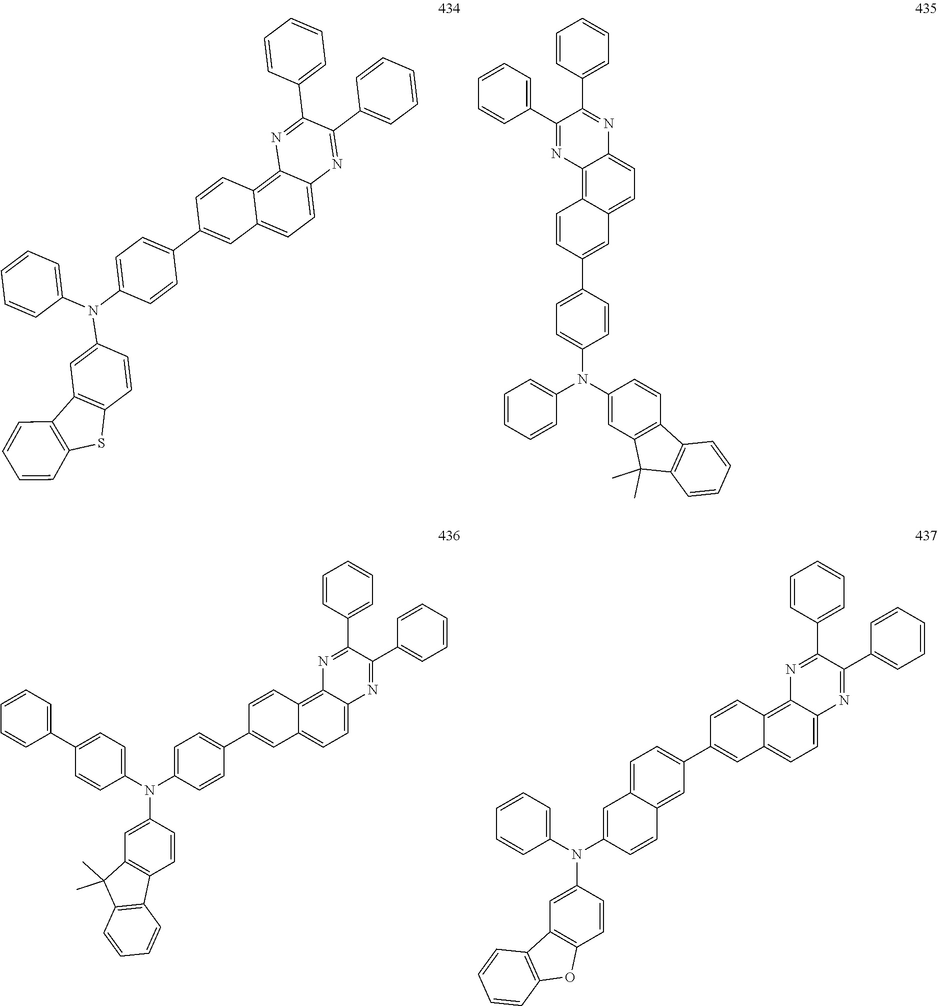 [SMILES: c1ccc(-c2nc3ccc4cc(-c5ccc(N(c6ccccc6)c6ccc7sc8ccccc8c7c6)cc5)ccc4c3nc2-c2ccccc2)cc1 ; CC1(C)c2ccccc2-c2ccc(N(c3ccccc3)c3ccc(-c4ccc5c(ccc6nc(-c7ccccc7)c(-c7ccccc7)nc65)c4)cc3)cc21 ; CC1(C)c2ccccc2-c2ccc(N(c3ccc(-c4ccccc4)cc3)c3ccc(-c4ccc5c(ccc6nc(-c7ccccc7)c(-c7ccccc7)nc65)c4)cc3)cc21 ; c1ccc(-c2nc3ccc4cc(-c5ccc6cc(N(c7ccccc7)c7ccc8oc9ccccc9c8c7)ccc6c5)ccc4c3nc2-c2ccccc2)cc1]

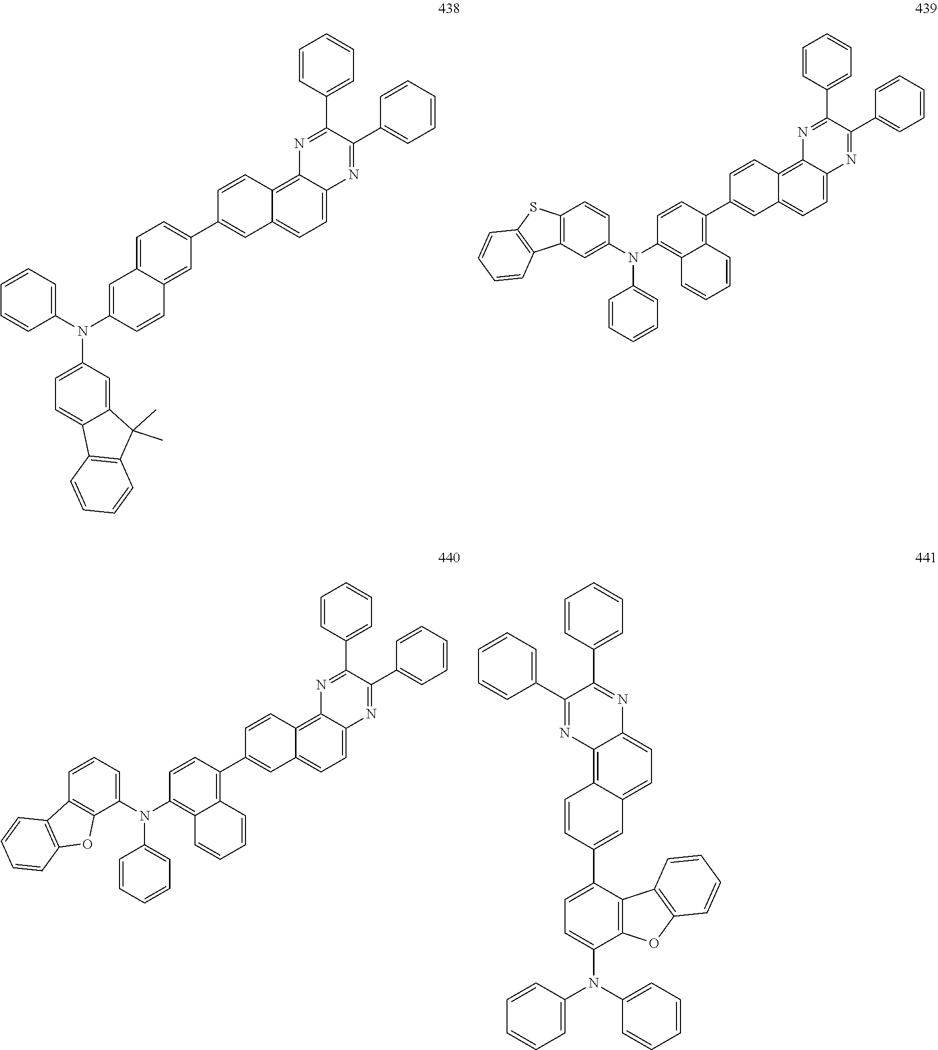

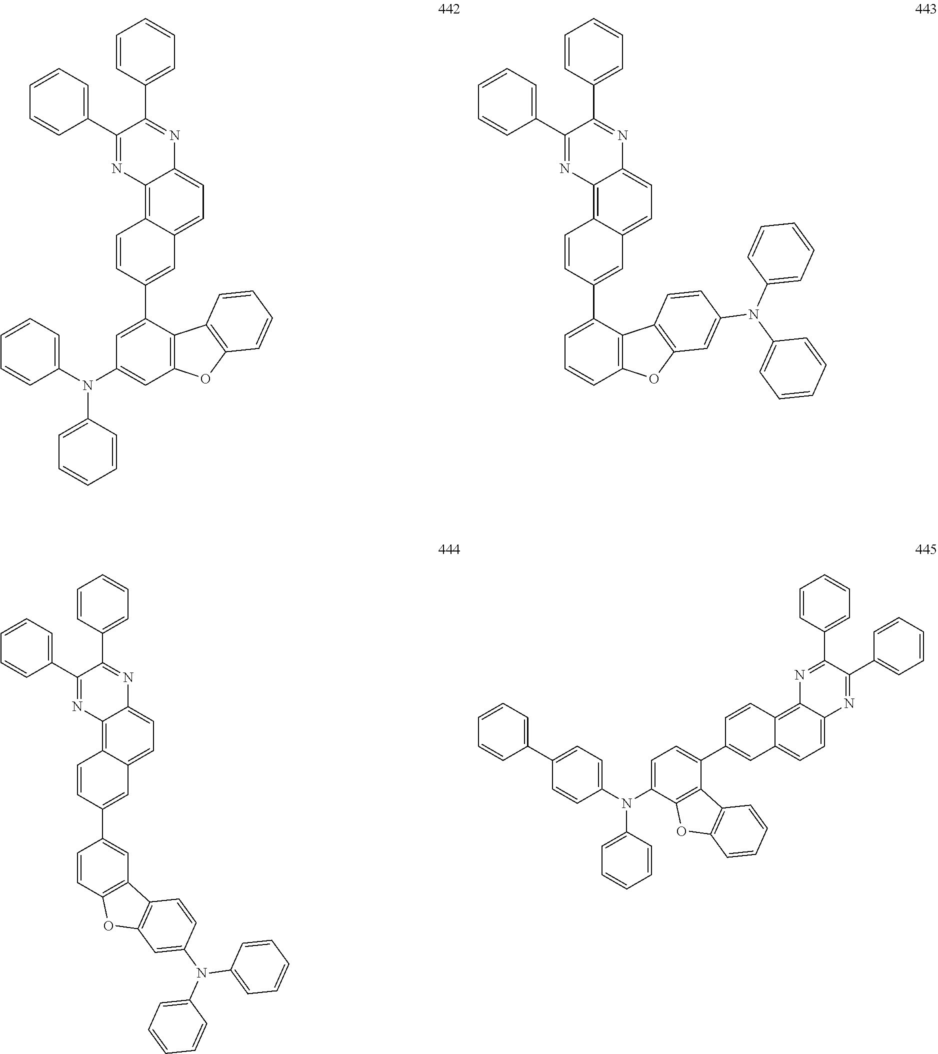 [SMILES: c1ccc(-c2nc3ccc4cc(-c5cc(N(c6ccccc6)c6ccccc6)cc6oc7ccccc7c56)ccc4c3nc2-c2ccccc2)cc1 ; c1ccc(-c2nc3ccc4cc(-c5cccc6oc7cc(N(c8ccccc8)c8ccccc8)ccc7c56)ccc4c3nc2-c2ccccc2)cc1 ; c1ccc(-c2nc3ccc4cc(-c5ccc6oc7cc(N(c8ccccc8)c8ccccc8)ccc7c6c5)ccc4c3nc2-c2ccccc2)cc1 ; c1ccc(-c2ccc(N(c3ccccc3)c3ccc(-c4ccc5c(ccc6nc(-c7ccccc7)c(-c7ccccc7)nc65)c4)c4c3oc3ccccc34)cc2)cc1]

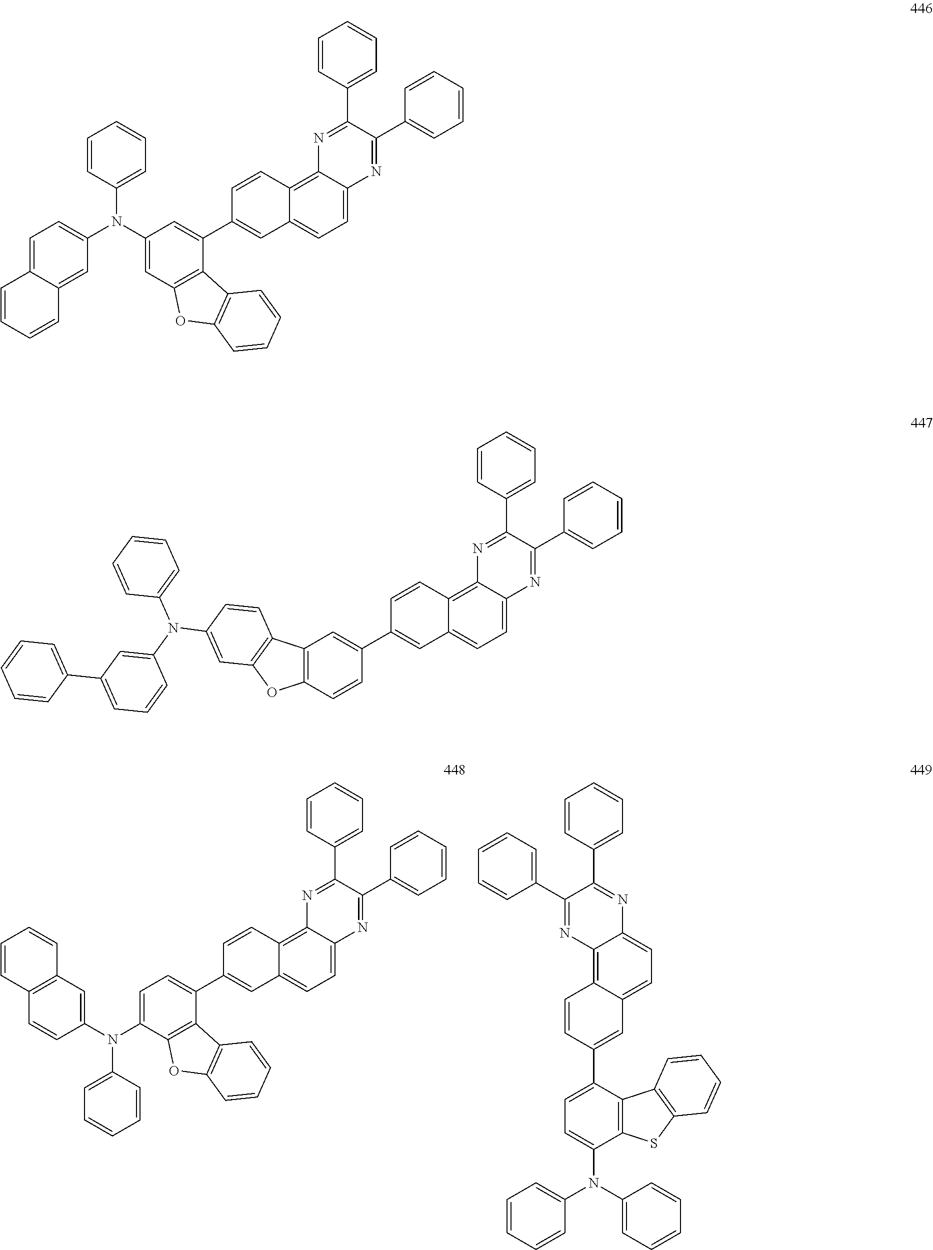

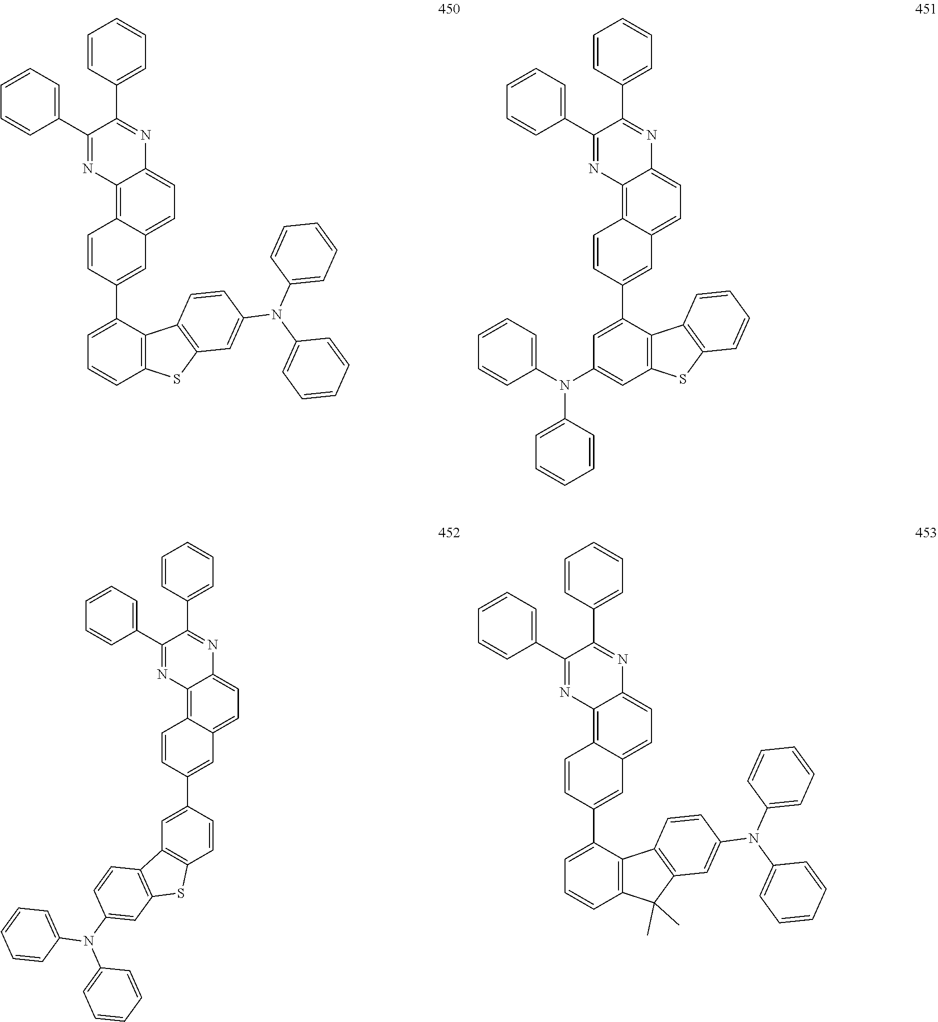 [SMILES: c1ccc(-c2nc3ccc4cc(-c5cccc6sc7cc(N(c8ccccc8)c8ccccc8)ccc7c56)ccc4c3nc2-c2ccccc2)cc1 ; c1ccc(-c2nc3ccc4cc(-c5cc(N(c6ccccc6)c6ccccc6)cc6sc7ccccc7c56)ccc4c3nc2-c2ccccc2)cc1 ; c1ccc(-c2nc3ccc4cc(-c5ccc6sc7cc(N(c8ccccc8)c8ccccc8)ccc7c6c5)ccc4c3nc2-c2ccccc2)cc1 ; CC1(C)c2cc(N(c3ccccc3)c3ccccc3)ccc2-c2c(-c3ccc4c(ccc5nc(-c6ccccc6)c(-c6ccccc6)nc54)c3)cccc21]

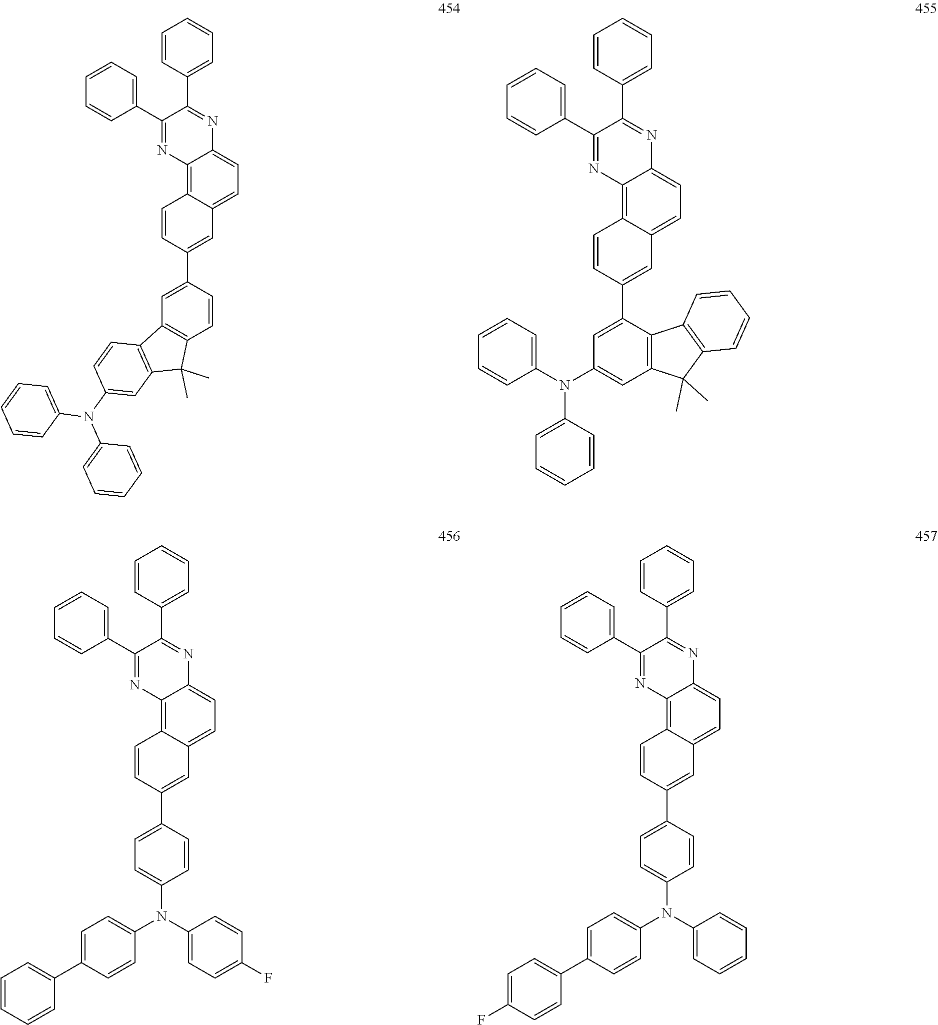 [SMILES: CC1(C)c2ccc(-c3ccc4c(ccc5nc(-c6ccccc6)c(-c6ccccc6)nc54)c3)cc2-c2ccc(N(c3ccccc3)c3ccccc3)cc21 ; CC1(C)c2ccccc2-c2c(-c3ccc4c(ccc5nc(-c6ccccc6)c(-c6ccccc6)nc54)c3)cc(N(c3ccccc3)c3ccccc3)cc21 ; Fc1ccc(N(c2ccc(-c3ccccc3)cc2)c2ccc(-c3ccc4c(ccc5nc(-c6ccccc6)c(-c6ccccc6)nc54)c3)cc2)cc1 ; Fc1ccc(-c2ccc(N(c3ccccc3)c3ccc(-c4ccc5c(ccc6nc(-c7ccccc7)c(-c7ccccc7)nc65)c4)cc3)cc2)cc1]

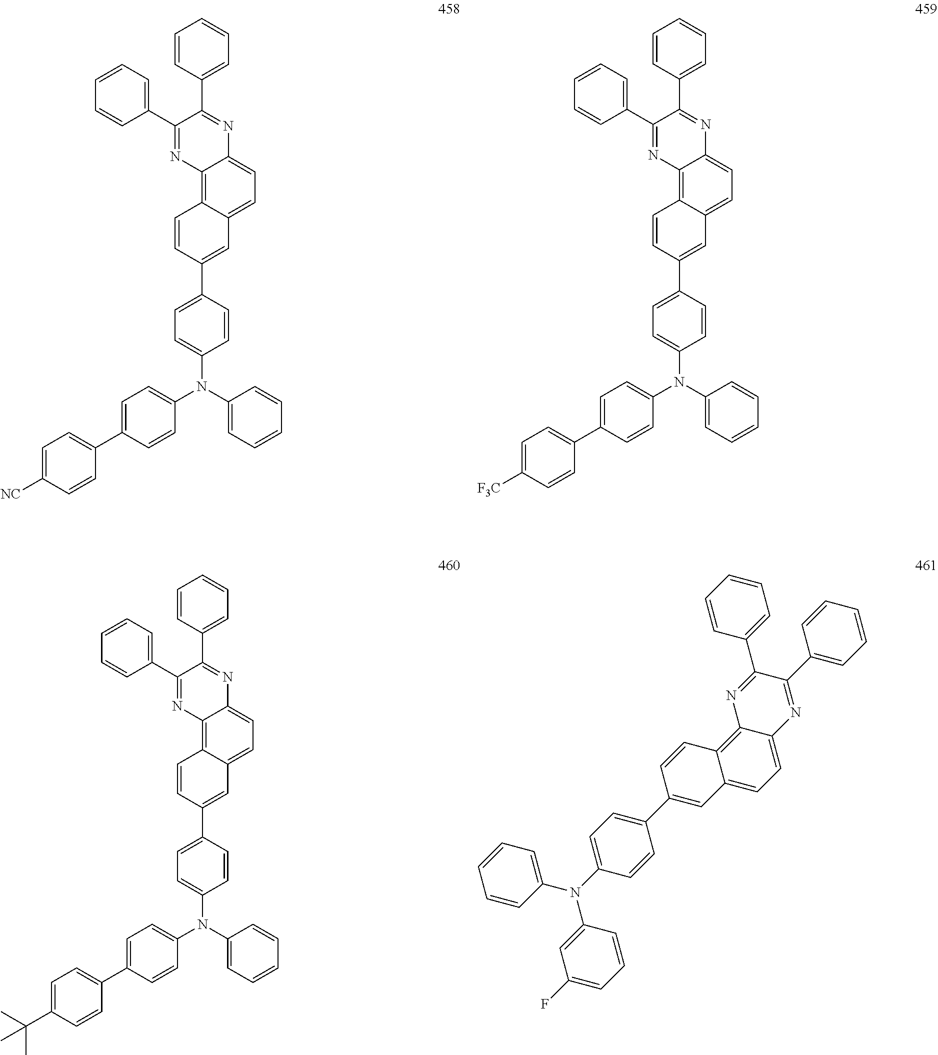 [SMILES: N#Cc1ccc(-c2ccc(N(c3ccccc3)c3ccc(-c4ccc5c(ccc6nc(-c7ccccc7)c(-c7ccccc7)nc65)c4)cc3)cc2)cc1 ; FC(F)(F)c1ccc(-c2ccc(N(c3ccccc3)c3ccc(-c4ccc5c(ccc6nc(-c7ccccc7)c(-c7ccccc7)nc65)c4)cc3)cc2)cc1 ; CC(C)(C)c1ccc(-c2ccc(N(c3ccccc3)c3ccc(-c4ccc5c(ccc6nc(-c7ccccc7)c(-c7ccccc7)nc65)c4)cc3)cc2)cc1 ; Fc1cccc(N(c2ccccc2)c2ccc(-c3ccc4c(ccc5nc(-c6ccccc6)c(-c6ccccc6)nc54)c3)cc2)c1]

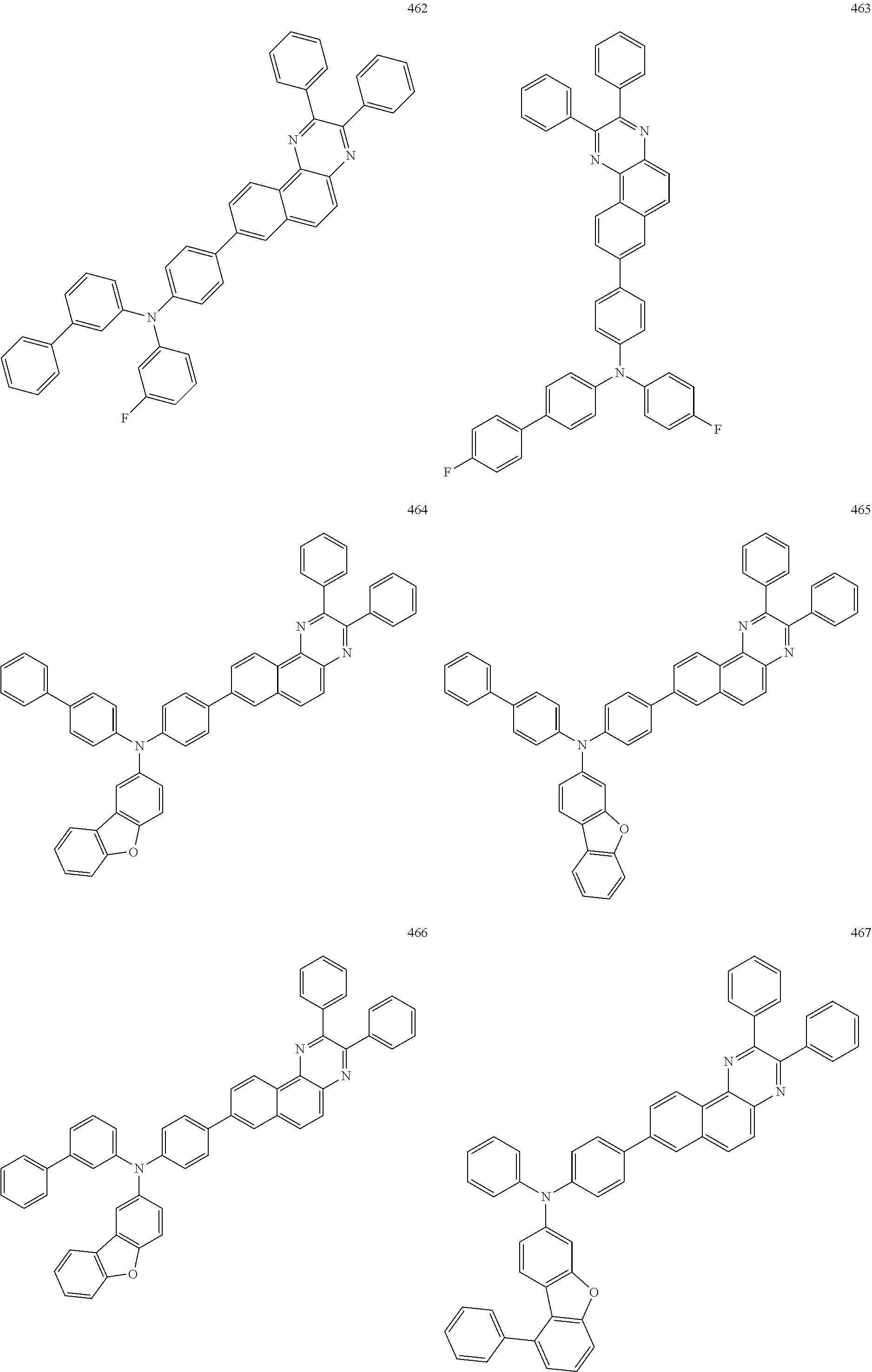

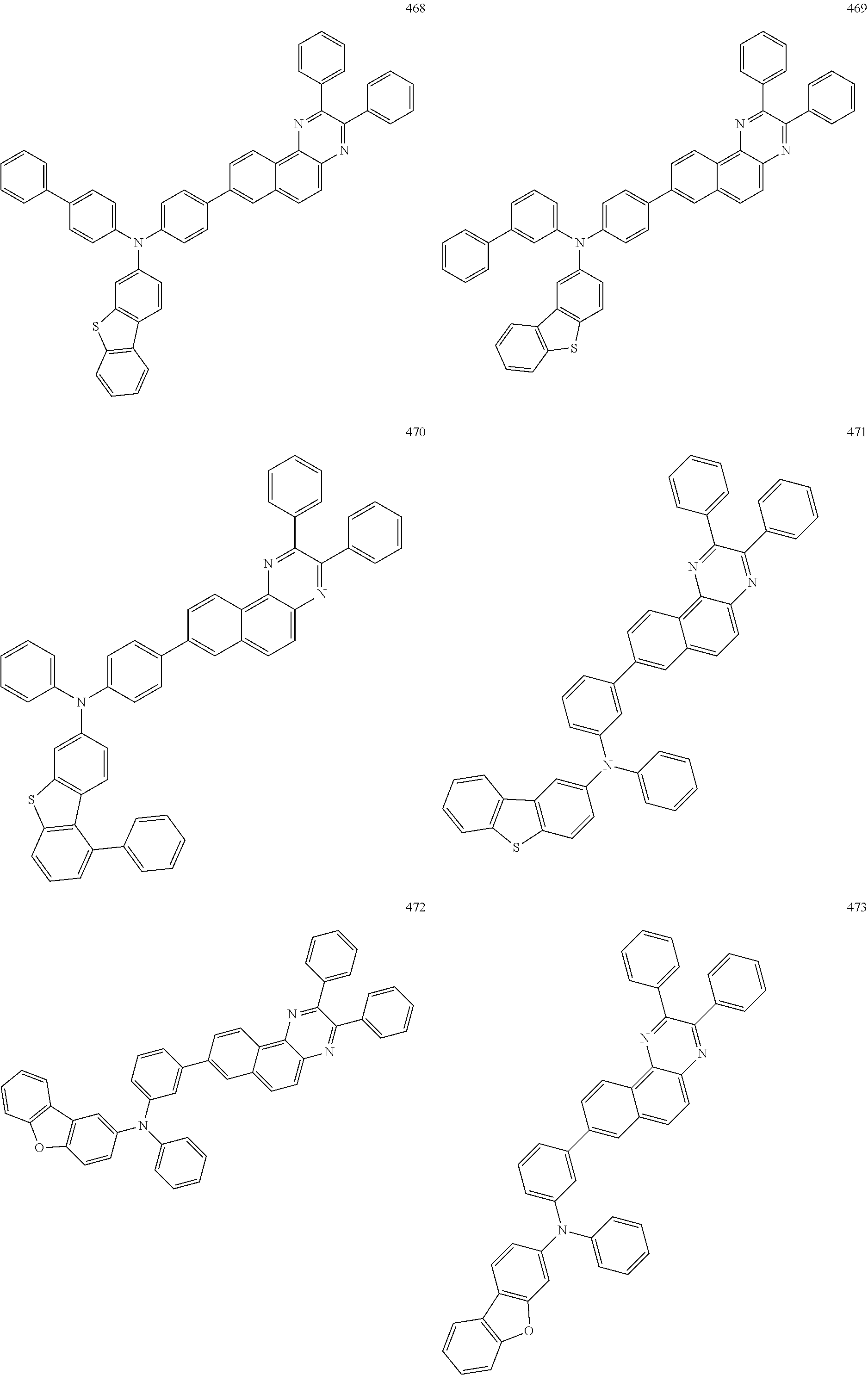

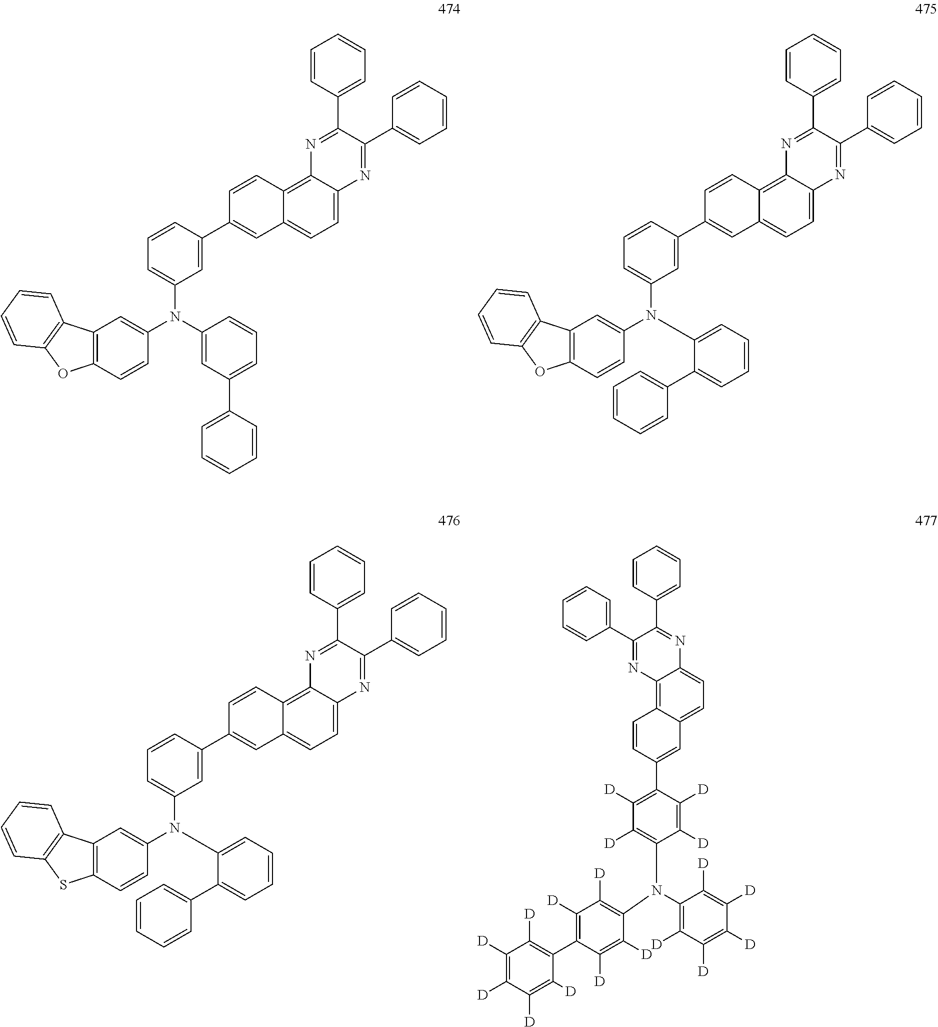 [SMILES: c1ccc(-c2cccc(N(c3cccc(-c4ccc5c(ccc6nc(-c7ccccc7)c(-c7ccccc7)nc65)c4)c3)c3ccc4oc5ccccc5c4c3)c2)cc1 ; c1ccc(-c2ccccc2N(c2cccc(-c3ccc4c(ccc5nc(-c6ccccc6)c(-c6ccccc6)nc54)c3)c2)c2ccc3oc4ccccc4c3c2)cc1 ; c1ccc(-c2ccccc2N(c2cccc(-c3ccc4c(ccc5nc(-c6ccccc6)c(-c6ccccc6)nc54)c3)c2)c2ccc3sc4ccccc4c3c2)cc1 ; [2H]c1c([2H])c([2H])c(-c2c([2H])c([2H])c(N(c3c([2H])c([2H])c([2H])c([2H])c3[2H])c3c([2H])c([2H])c(-c4ccc5c(ccc6nc(-c7ccccc7)c(-c7ccccc7)nc65)c4)c([2H])c3[2H])c([2H])c2[2H])c([2H])c1[2H]]

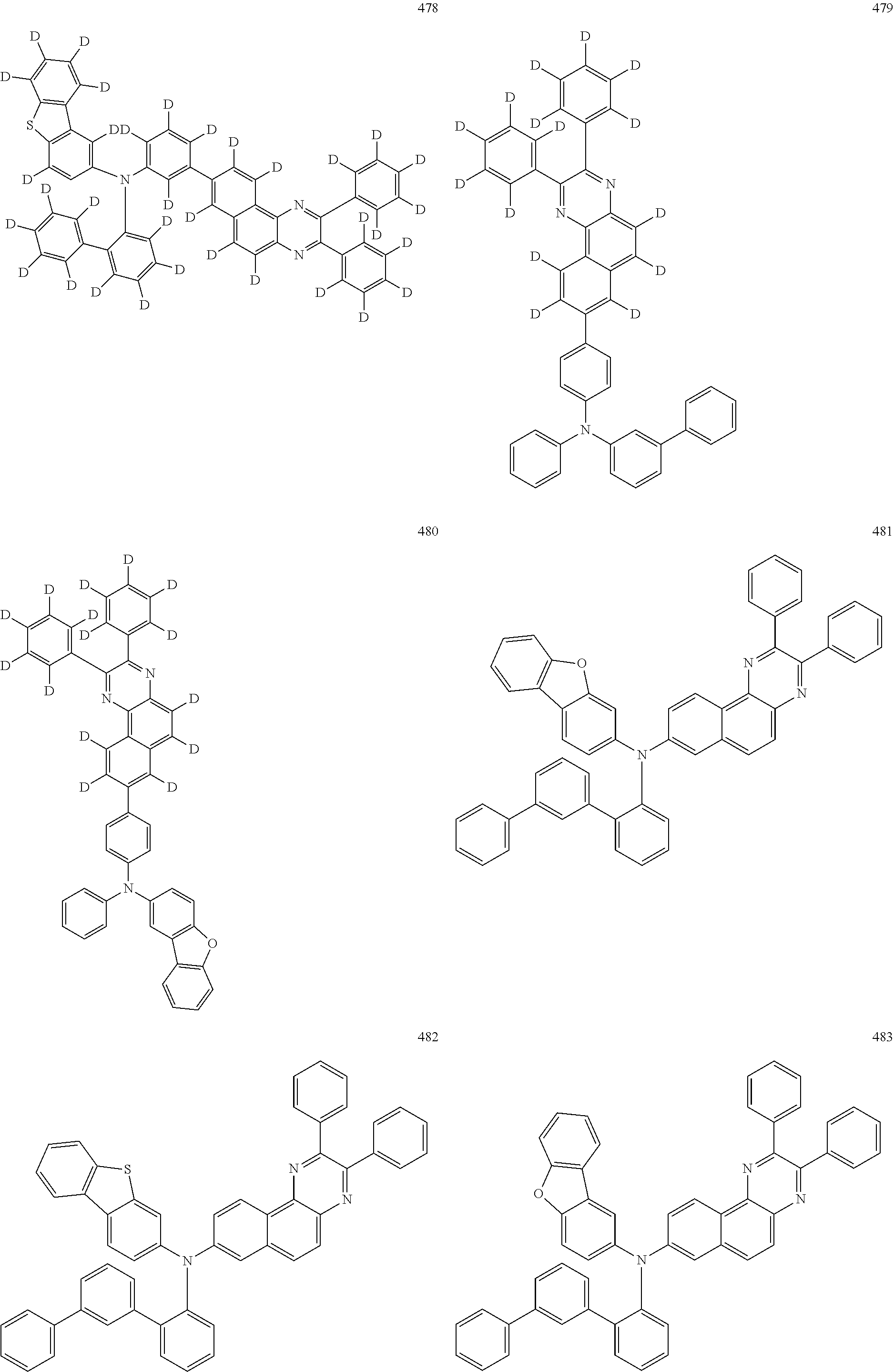

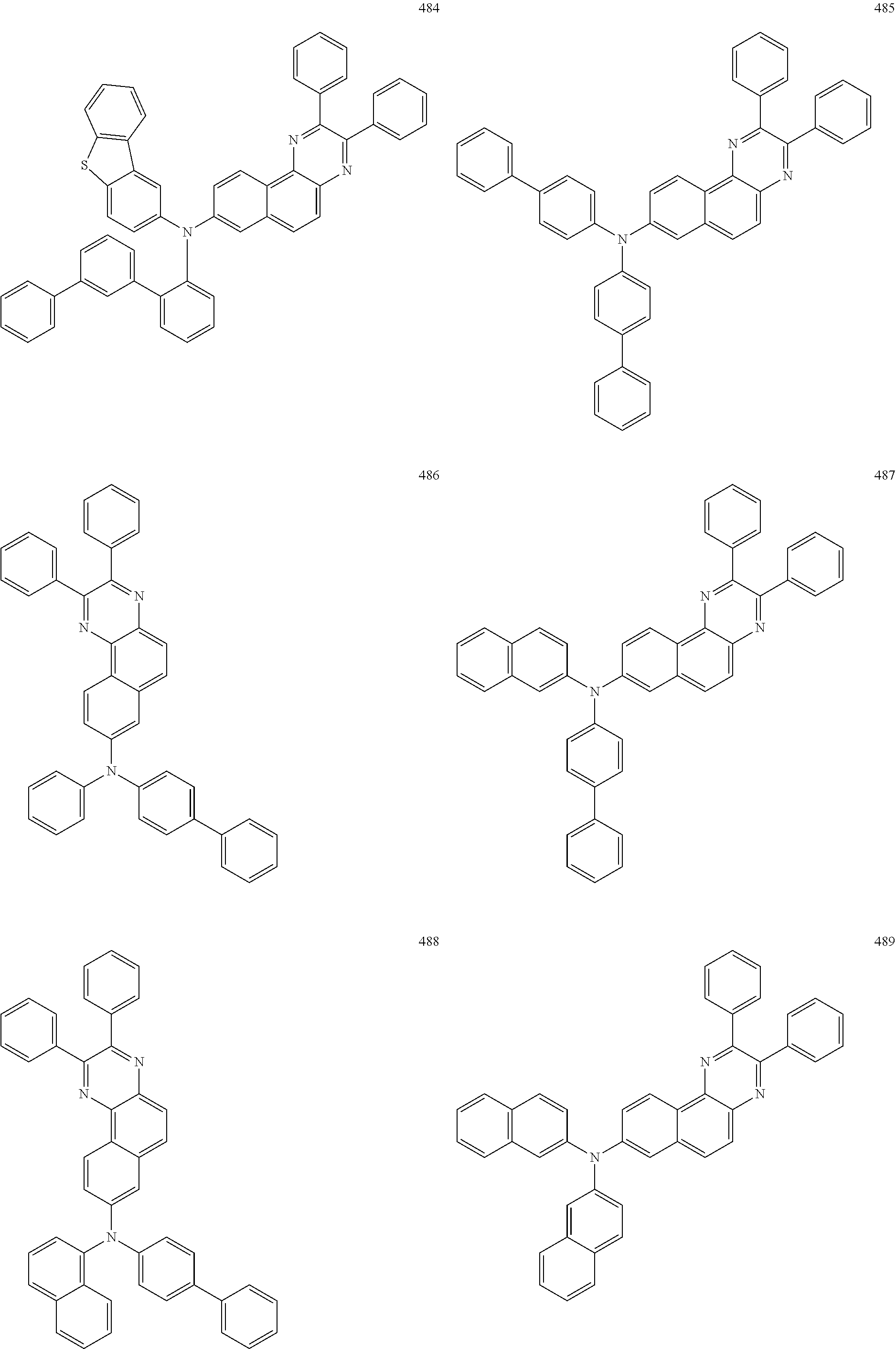

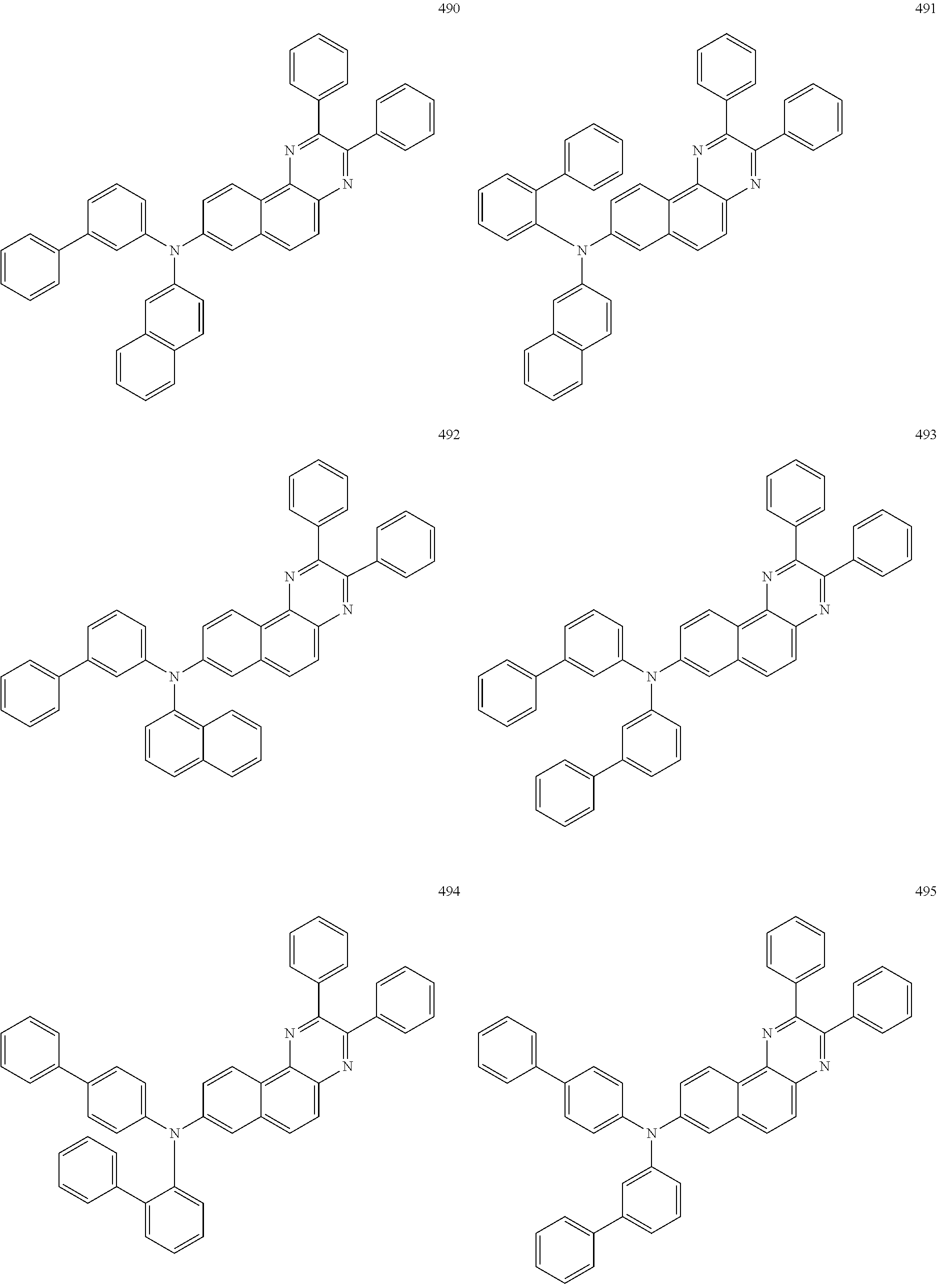

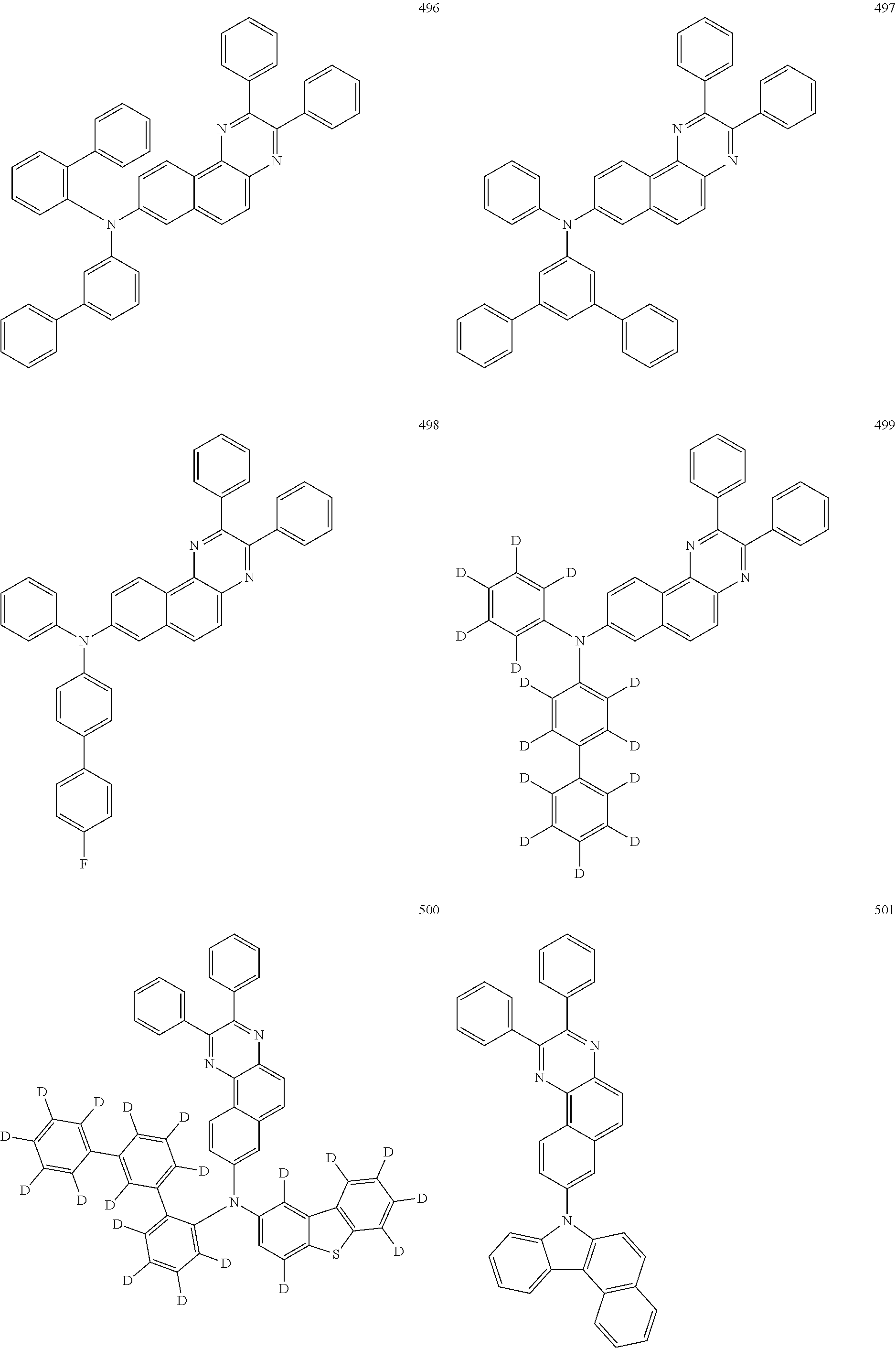

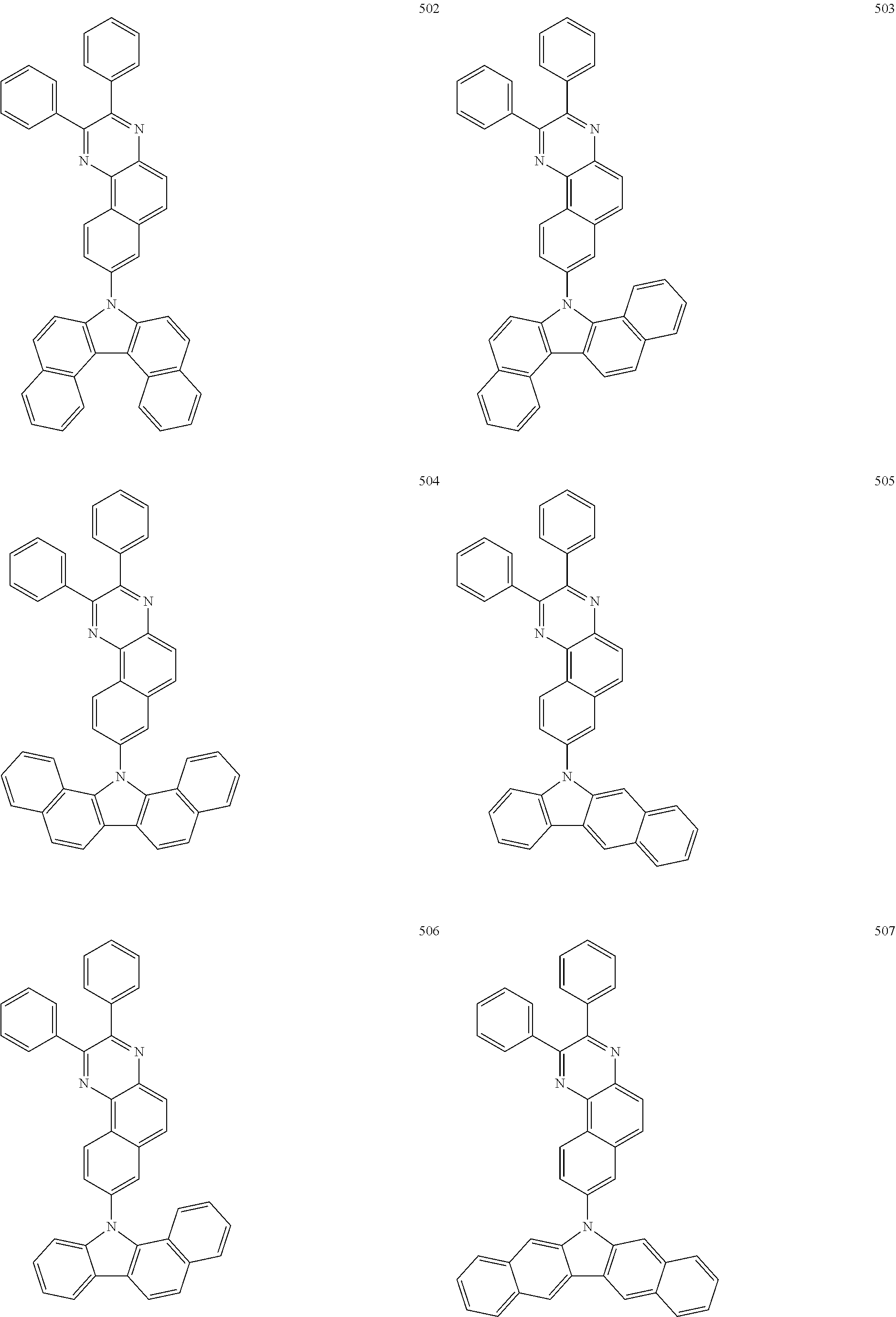 [SMILES: c1ccc(-c2nc3ccc4cc(-n5c6ccc7ccccc7c6c6c7ccccc7ccc65)ccc4c3nc2-c2ccccc2)cc1 ; c1ccc(-c2nc3ccc4cc(-n5c6ccc7ccccc7c6c6ccc7ccccc7c65)ccc4c3nc2-c2ccccc2)cc1 ; c1ccc(-c2nc3ccc4cc(-n5c6c7ccccc7ccc6c6ccc7ccccc7c65)ccc4c3nc2-c2ccccc2)cc1 ; c1ccc(-c2nc3ccc4cc(-n5c6ccccc6c6cc7ccccc7cc65)ccc4c3nc2-c2ccccc2)cc1 ; c1ccc(-c2nc3ccc4cc(-n5c6ccccc6c6ccc7ccccc7c65)ccc4c3nc2-c2ccccc2)cc1 ; c1ccc(-c2nc3ccc4cc(-n5c6cc7ccccc7cc6c6cc7ccccc7cc65)ccc4c3nc2-c2ccccc2)cc1]

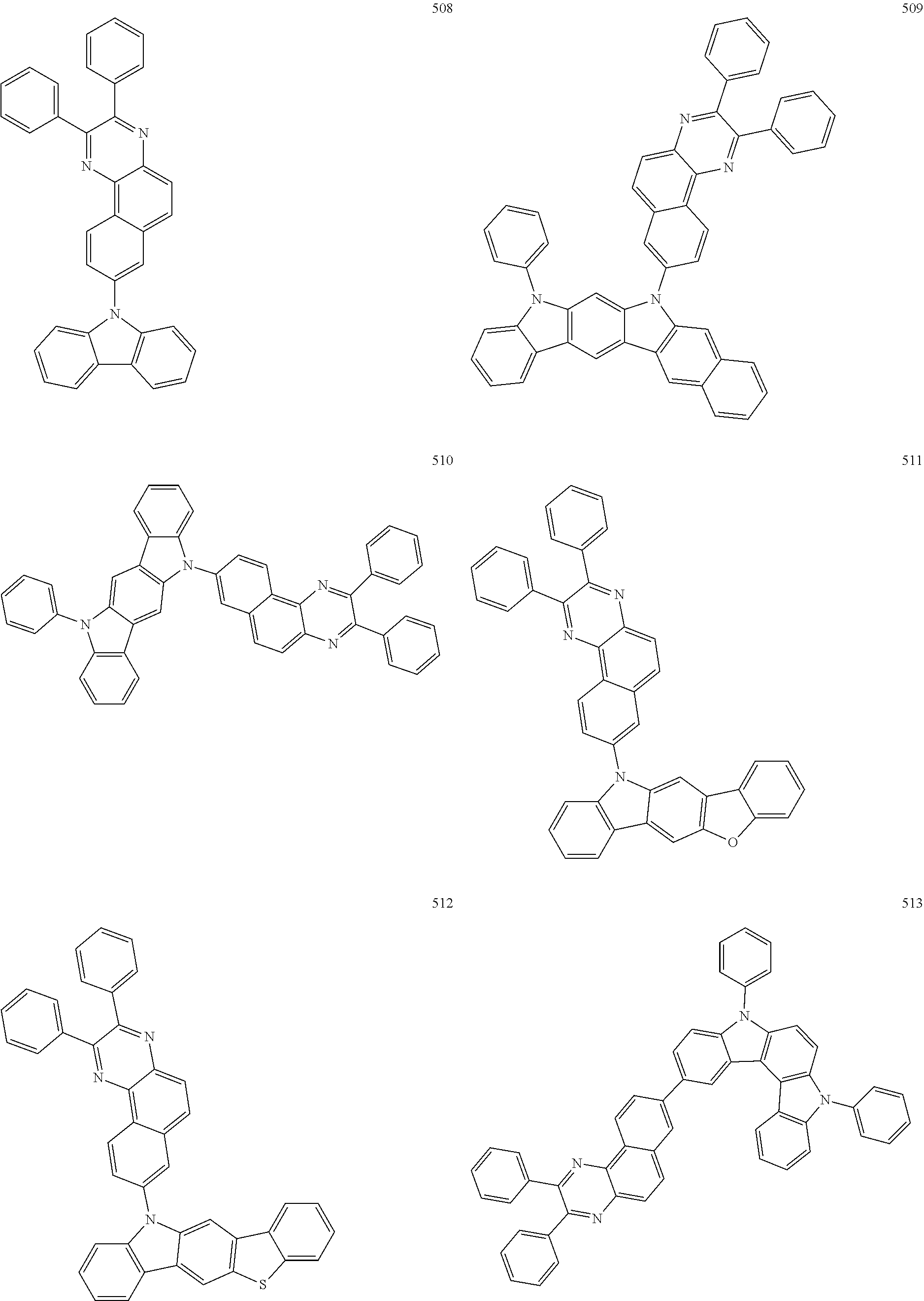 [SMILES: c1ccc(-c2nc3ccc4cc(-n5c6ccccc6c6ccccc65)ccc4c3nc2-c2ccccc2)cc1 ; c1ccc(-c2nc3ccc4cc(-n5c6cc7ccccc7cc6c6cc7c8ccccc8n(-c8ccccc8)c7cc65)ccc4c3nc2-c2ccccc2)cc1 ; c1ccc(-c2nc3ccc4cc(-n5c6ccccc6c6cc7c(cc65)c5ccccc5n7-c5ccccc5)ccc4c3nc2-c2ccccc2)cc1 ; c1ccc(-c2nc3ccc4cc(-n5c6ccccc6c6cc7oc8ccccc8c7cc65)ccc4c3nc2-c2ccccc2)cc1 ; c1ccc(-c2nc3ccc4cc(-n5c6ccccc6c6cc7sc8ccccc8c7cc65)ccc4c3nc2-c2ccccc2)cc1 ; c1ccc(-c2nc3ccc4cc(-c5ccc6c(c5)c5c7c8ccccc8n(-c8ccccc8)c7ccc5n6-c5ccccc5)ccc4c3nc2-c2ccccc2)cc1]

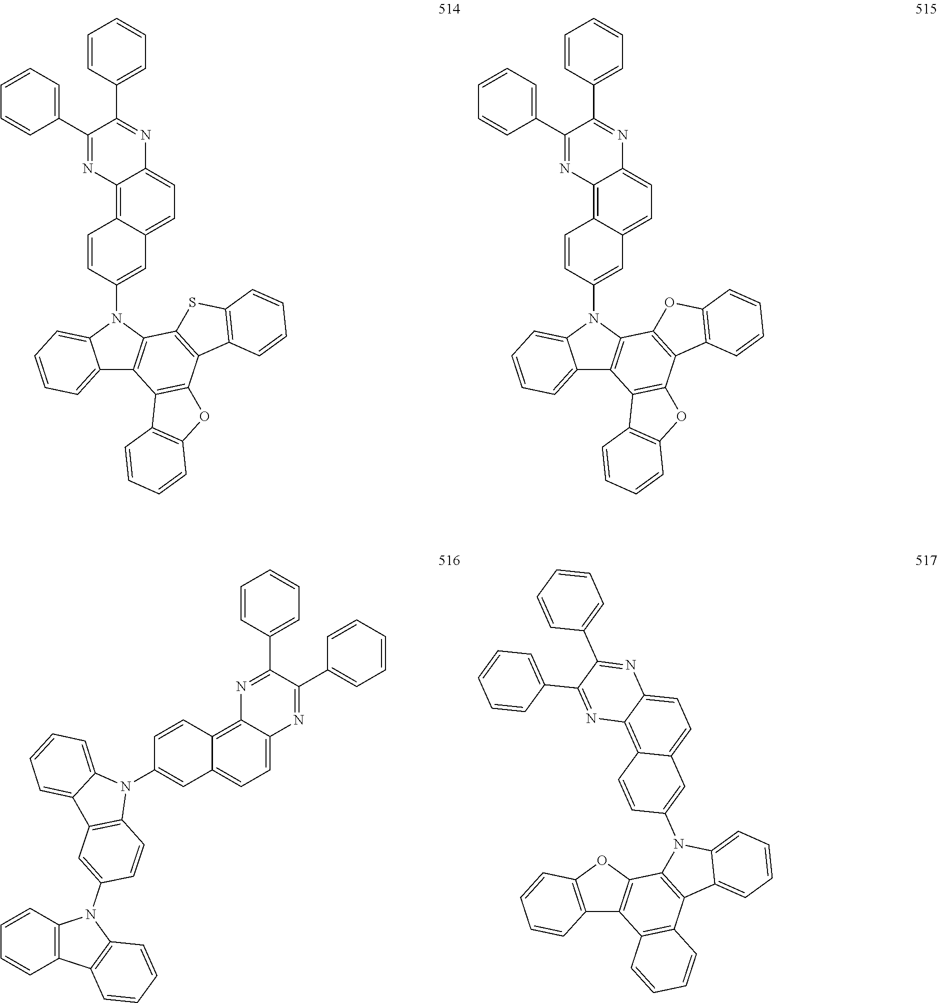 [SMILES: c1ccc(-c2nc3ccc4cc(-n5c6ccccc6c6c7c8ccccc8oc7c7c8ccccc8sc7c65)ccc4c3nc2-c2ccccc2)cc1 ; c1ccc(-c2nc3ccc4cc(-n5c6ccccc6c6c7c8ccccc8oc7c7c8ccccc8oc7c65)ccc4c3nc2-c2ccccc2)cc1 ; c1ccc(-c2nc3ccc4cc(-n5c6ccccc6c6cc(-n7c8ccccc8c8ccccc87)ccc65)ccc4c3nc2-c2ccccc2)cc1 ; c1ccc(-c2nc3ccc4cc(-n5c6ccccc6c6c7ccccc7c7c8ccccc8oc7c65)ccc4c3nc2-c2ccccc2)cc1]

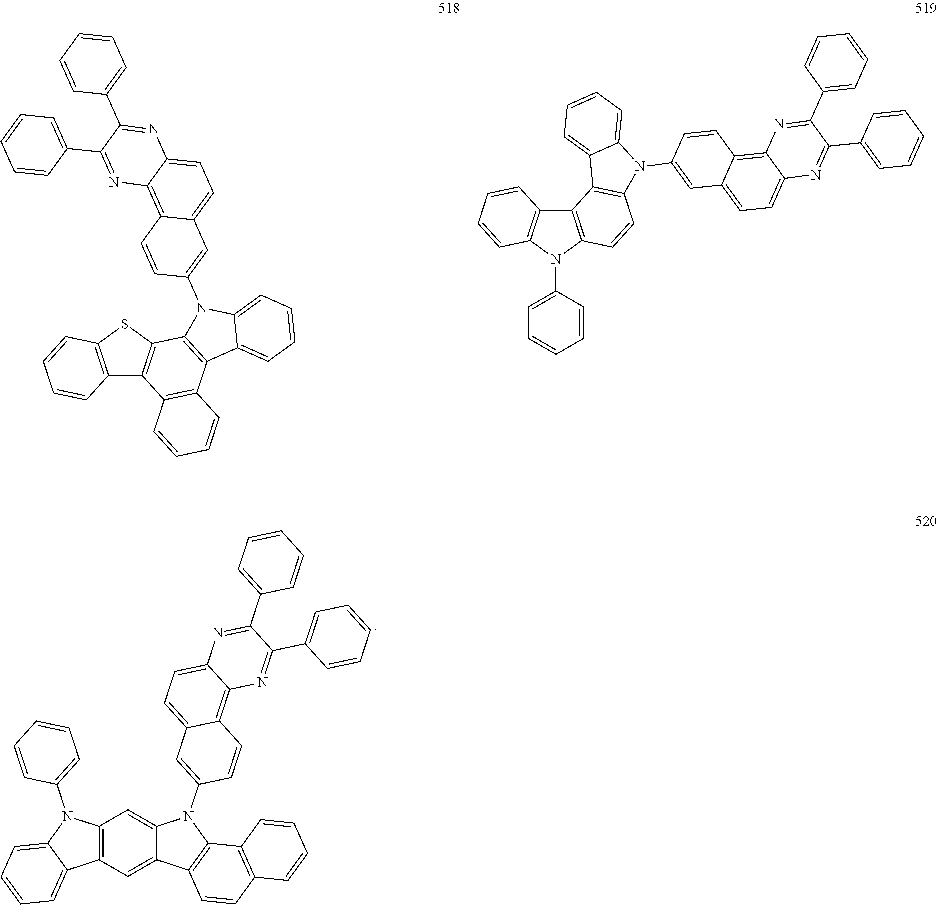 [SMILES: c1ccc(-c2nc3ccc4cc(-n5c6ccccc6c6c7ccccc7c7c8ccccc8sc7c65)ccc4c3nc2-c2ccccc2)cc1 ; c1ccc(-c2nc3ccc4cc(-n5c6ccccc6c6c7c8ccccc8n(-c8ccccc8)c7ccc65)ccc4c3nc2-c2ccccc2)cc1 ; c1ccc(-c2nc3ccc4cc(-n5c6cc7c(cc6c6ccc8ccccc8c65)c5ccccc5n7-c5ccccc5)ccc4c3nc2-c2ccccc2)cc1]

In addition, by introducing various substituents to the structure of Chemical Formula 1, compounds having unique properties of the introduced substituents may be synthesized. For example, by introducing substituents normally used for a hole injection layer material, a hole transport layer material, a hole transport auxiliary layer material, an electron blocking layer material, a light emitting layer material, an electron transport layer material, a hole blocking layer material and an electron injection layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials may be enhanced, and material applications may become diverse.

One embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

In addition, one embodiment of the present disclosure provides an organic light emitting device including: a first electrode; a second electrode provided opposite to the first electrode; and one or more organic material layers provided between the first electrode and the second electrode, wherein one or more layers of the organic material layers include the heterocyclic compound represented by Chemical Formula 1.

In one embodiment of the present disclosure, the first electrode may be a positive electrode, and the second electrode may be a negative electrode.

In one embodiment of the present disclosure, the first electrode may be a negative electrode, and the second electrode may be a positive electrode.

In one embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a green organic light emitting device, and the heterocyclic compound represented by Chemical Formula 1 may be used as a material of the green organic light emitting device.

In another embodiment of the present disclosure, the organic light emitting device may be a blue organic light emitting device, and the heterocyclic compound represented by Chemical Formula 1 may be used as a material of the blue organic light emitting device.

In one embodiment of the present disclosure, the organic light emitting device may be a red organic light emitting device, and the heterocyclic compound represented by Chemical Formula 1 may be used as a light emitting layer material of the red organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may form the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a hole transport auxiliary layer, a light emitting layer, an electron injection layer, an electron transport layer, an electron blocking layer, a hole blocking layer and the like as the organic material layer. However, the structure of the organic light emitting device is not limited thereto, and may include a smaller number of organic material layers.

In the organic light emitting device of the present disclosure, the organic material layer includes a light emitting layer, and the light emitting layer may include the heterocyclic compound represented by Chemical Formula 1. When the heterocyclic compound is used in the light emitting layer, HOMO (highest occupied molecular orbital) and LUMO (lowest unoccupied molecular orbital) are spatially separated, enabling strong charge transfer, and therefore, driving efficiency and lifetime of the organic light emitting device may become superior.

In one embodiment of the present disclosure, the organic light emitting device may include one or more organic material layers, the organic material layer may include a light emitting layer, and the light emitting layer may include the heterocyclic compound represented by Chemical Formula 1.

In another embodiment of the present disclosure, the organic material layer may include a light emitting layer, the light emitting layer may include a host material, and the host material may include the heterocyclic compound.

In another embodiment of the present disclosure, the organic light emitting device may further include, one, two or more layers selected from the group consisting of a light emitting layer, a hole injection layer, a hole transport layer, an electron injection layer, an electron transport layer, an electron blocking layer and a hole blocking layer.

In one embodiment of the present disclosure, the organic light emitting device may include one or more organic material layers, the organic material layer may include a hole transport layer, and the hole transport layer may include the heterocyclic compound represented by Chemical Formula 1.

In another embodiment of the present disclosure, the organic light emitting device may further include, one, two or more layers selected from the group consisting of a light emitting layer, a hole injection layer, a hole transport auxiliary layer, an electron injection layer, an electron transport layer, an electron blocking layer and a hole blocking layer.

In one embodiment of the present disclosure, the organic light emitting device may include one or more organic material layers, the organic material layer may include a hole transport auxiliary layer, and the hole transport auxiliary layer may include the heterocyclic compound represented by Chemical Formula 1.

In another embodiment of the present disclosure, the organic light emitting device may further include, one, two or more layers selected from the group consisting of a light emitting layer, a hole injection layer, a hole transport layer, an electron injection layer, an electron transport layer, an electron blocking layer and a hole blocking layer.

In one embodiment of the present disclosure, the organic light emitting device may include one or more organic material layers, the organic material layer may include an electron blocking layer, and the electron blocking layer may include the heterocyclic compound represented by Chemical Formula 1.

In another embodiment of the present disclosure, the organic light emitting device may further include, one, two or more layers selected from the group consisting of a light emitting layer, a hole injection layer, a hole transport layer, a hole transport auxiliary layer, an electron injection layer, an electron transport layer and a hole blocking layer.

In one embodiment of the present disclosure, the organic material layer includes the heterocyclic compound represented by Chemical Formula 1, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited by these examples.

M may be iridium, platinum, osmium or the like.

L is an anionic bidentate ligand coordinated to M by sp² carbon and heteroatom, and X may function to trap electrons or holes. Nonlimiting examples of L, L′ and L″ may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples:

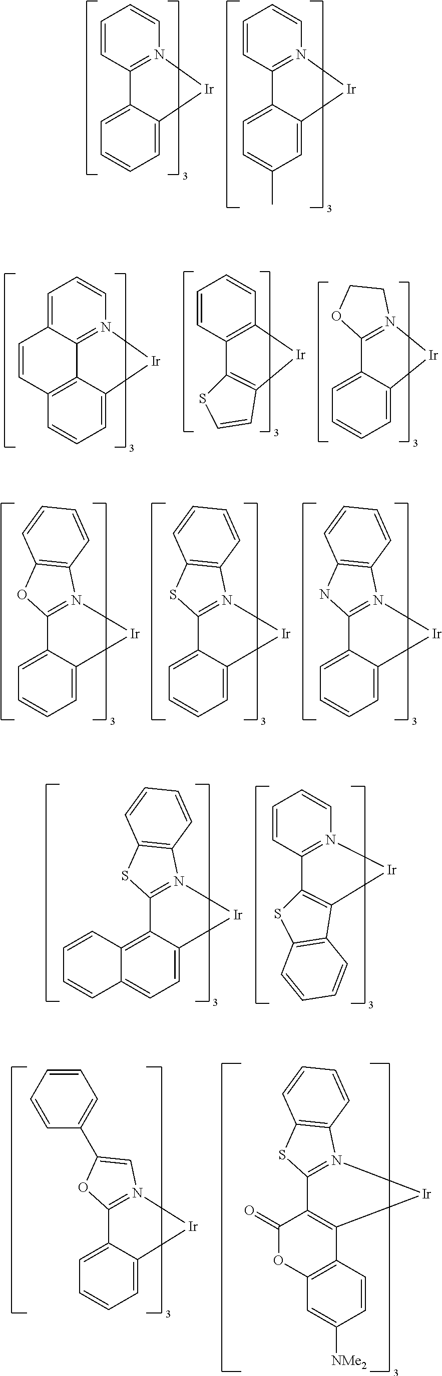

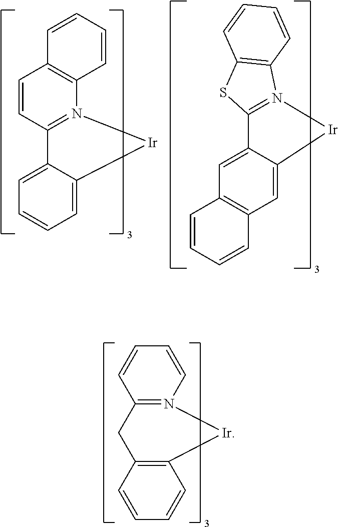

In one embodiment of the present disclosure, the organic material layer includes the heterocyclic compound represented by Chemical Formula 1, and an iridium-based dopant may be used therewith.

In one embodiment of the present disclosure, as the iridium-based dopant, (piq)₂(Ir) (acac) may be used as a red phosphorescent dopant.

In one embodiment of the present disclosure, a content of the dopant may be from 1% to 15%, preferably from 2% to 10% and more preferably from 3% to 7% based on the total weight of the light emitting layer.

In the organic light emitting device according to one embodiment of the present disclosure, the organic material layer includes a hole transport layer or a hole transport auxiliary layer, and the hole transport layer or the hole transport auxiliary layer may include the heterocyclic compound represented by Chemical Formula 1.

In the organic light emitting device according to another embodiment of the present disclosure, the organic material layer includes an electron injection layer or an electron transport layer, and the electron injection layer or the electron transport layer may include the heterocyclic compound represented by Chemical Formula 1.

In the organic light emitting device according to another embodiment of the present disclosure, the organic material layer includes an electron blocking layer or a hole blocking layer, and the electron blocking layer or the hole blocking layer may include the heterocyclic compound represented by Chemical Formula 1.

In the organic light emitting device according to another embodiment of the present disclosure, the organic material layer includes an electron transport layer, a light emitting layer or a hole blocking layer, and the electron transport layer, the light emitting layer or the hole blocking layer may include the heterocyclic compound represented by Chemical Formula 1.

In the organic light emitting device according to another embodiment of the present disclosure, the organic material layer includes a light emitting layer, and the light emitting layer may include the heterocyclic compound represented by Chemical Formula 1.

In the organic light emitting device according to another embodiment of the present disclosure, the organic material layer includes a light emitting layer, the light emitting layer includes a host material, and the host material may include the heterocyclic compound represented by Chemical Formula 1.

In the organic light emitting device according to still another embodiment, the light emitting layer may include two or more host materials, and at least one of the host materials may include the heterocyclic compound represented by Chemical Formula 1.

In the organic light emitting device according to yet another embodiment, two or more host materials may be pre-mixed and used in the light emitting layer, and at least one of the two or more host materials may include the heterocyclic compound represented by Chemical Formula 1.

The pre-mixing means, before depositing the two or more host materials on the organic material layer, putting and mixing the materials first in one source of supply.

The organic light emitting device according to one embodiment of the present disclosure may further include, one, two or more layers selected from the group consisting of a light emitting layer, a hole injection layer, a hole transport layer, an electron injection layer, an electron transport layer, an electron blocking layer and a hole blocking layer.

In the organic light emitting device according to one embodiment of the present disclosure, the organic material layer including the heterocyclic compound represented by Chemical Formula 1 further includes a heterocyclic compound represented by the following Chemical Formula A:

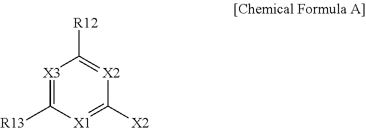

in Chemical Formula A,
X1 to X3 are the same as or different from each other, and each independently N or CR″,
at least two or more of X1 to X3 are N,
R11 to R13 and R″ are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heteroring, and
at least one of R11 to R13 is a group represented by the following Chemical Formula B-1 or Chemical Formula B-2,

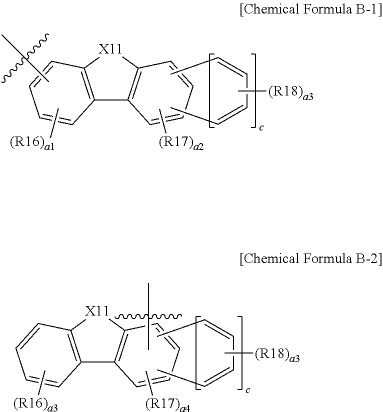

in Chemical Formulae B-1 and B-2,
X11 is O or S,
c is an integer of 0 or 1,
R16 to R18 are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group,
a1 is one of integers of 1 to 3,
a2 is one of integers of 1 to 4, and when c is 1, a2 may be 1 or 2,
a3 is one of integers of 1 to 4, and
a4 is one of integers of 1 to 3, and when c is 1, a4 may be 1.

In the organic light emitting device according to one embodiment of the present disclosure, the heterocyclic compound represented by Chemical Formula A may be represented by any one of the following Chemical Formula A-1, Chemical Formula A-2 and Chemical Formula A-3:

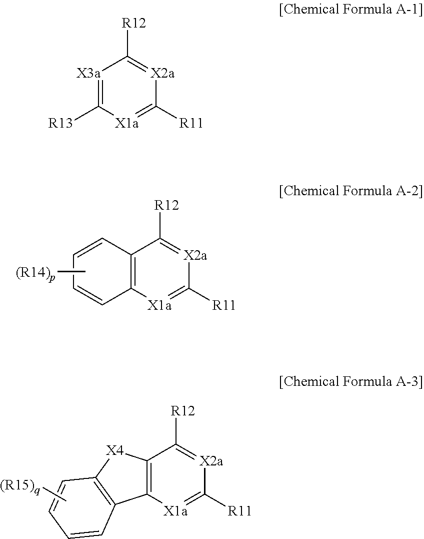

in Chemical Formula A-1 to Chemical Formula A-3,
X1a to X3a have the same definitions as X1 to X3 of Chemical Formula A,
X4 is O or S,
R11 to R15 are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, and
p is one of integers of 1 to 4, and q is one of integers of 1 to 4.

In one embodiment of the present disclosure, X1 to X3 and X1a to X3a are N; or CR″, and at least two or more of X1 to X3 may be N.

In one embodiment of the present disclosure, X1 and X2 are N, and X3 may be CR″.

In one embodiment of the present disclosure, X1 and X3 are N, and X2 may be CR″.

In one embodiment of the present disclosure, X1a and X2a are N, and X3a may be CR″.

In one embodiment of the present disclosure, X1a and X3a are N, and X2a may be CR″.

In one embodiment of the present disclosure, X4 may be O or S.

In one embodiment of the present disclosure, X4 may be S.

In one embodiment of the present disclosure, R11 to R15 and R″ are the same as or different from each other, and may be each independently selected from the group consisting of hydrogen; deuterium; a halogen group; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; and a substituted or unsubstituted C2 to C40 heteroaryl group.

In one embodiment of the present disclosure, R11 to R15 and R″ are the same as or different from each other, and may be each independently selected from the group consisting of hydrogen; deuterium; a halogen group; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group.

In one embodiment of the present disclosure, R11 to R15 and R″ are the same as or different from each other, and may be each independently selected from the group consisting of hydrogen; deuterium; a halogen group; a cyano group; a substituted or unsubstituted C6 to C20 aryl group; and a substituted or unsubstituted C2 to C20 heteroaryl group.

In one embodiment of the present disclosure, R11 to R15 and R″ are the same as or different from each other, and may be each independently selected from the group consisting of a substituted or unsubstituted C6 to C20 aryl group; and a substituted or unsubstituted C2 to C20 heteroaryl group.

In one embodiment of the present disclosure, two or more groups of R11 to R13 and R″ adjacent to each other may bond to each other to form a substituted or unsubstituted C6 to C60 aromatic hydrocarbon ring; or a substituted or unsubstituted C2 to C60 heteroring.

In one embodiment of the present disclosure, two or more groups of R11 to R13 and R″ adjacent to each other may bond to each other to form a substituted or unsubstituted C6 to C40 aromatic hydrocarbon ring; or a substituted or unsubstituted C2 to C40 heteroring.

In one embodiment of the present disclosure, two or more groups of R11 to R13 and R″ adjacent to each other may bond to each other to form a substituted or unsubstituted C6 to C20 aromatic hydrocarbon ring; or a substituted or unsubstituted C2 to C20 heteroring.

In one embodiment of the present disclosure, at least one of R11 to R13 may be a group represented by the following Chemical Formula B-1 or Chemical Formula B-2:

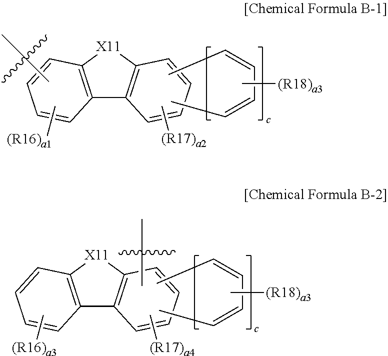

in Chemical Formulae B-1 and B-2,
X11 is O or S,
c is an integer of 0 or 1,
R16 to R18 are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group,
a1 is one of integers of 1 to 3,
a2 is one of integers of 1 to 4, and when c is 1, a2 may be 1 or 2,
a3 is one of integers of 1 to 4, and
a4 is one of integers of 1 to 3, and when c is 1, a4 may be 1.

In one embodiment of the present disclosure, X11 may be O or S.

In one embodiment of the present disclosure, c may be 0 or 1.

In one embodiment of the present disclosure, R16 to R18 are the same as or different from each other, and may be each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

In one embodiment of the present disclosure, R16 to R18 are the same as or different from each other, and may be each independently selected from the group consisting of hydrogen; deuterium; a halogen group; a cyano group; a substituted or unsubstituted C6 to C60 aryl group; and a substituted or unsubstituted C2 to C60 heteroaryl group.

In one embodiment of the present disclosure, R16 to R18 are the same as or different from each other, and may be each independently selected from the group consisting of hydrogen; deuterium; a halogen group; a cyano group; a substituted or unsubstituted C6 to C30 aryl group; and a substituted or unsubstituted C2 to C30 heteroaryl group.

In one embodiment of the present disclosure, R16 to R18 are the same as or different from each other, and may be each independently selected from the group consisting of hydrogen; deuterium; a substituted or unsubstituted C6 to C20 aryl group; and a substituted or unsubstituted C2 to C20 heteroaryl group.

In one embodiment of the present disclosure, R16 to R18 are the same as or different from each other and may be each independently hydrogen; deuterium; a substituted or unsubstituted phenyl group; a substituted or unsubstituted biphenyl group; a substituted or unsubstituted terphenyl group; a substituted or unsubstituted naphthyl group; a substituted or unsubstituted dibenzothiophenyl group; or a substituted or unsubstituted dibenzofuranyl group, but are not limited to these examples.

In one embodiment of the present disclosure, a1 may be 1, 2 or 3.

In one embodiment of the present disclosure, a2 may be 1, 2, 3 or 4, and when c is 1, a2 may be 1 or 2.

In one embodiment of the present disclosure, a3 may be 1, 2, 3 or 4.

In one embodiment of the present disclosure, a4 may be 1, 2 or 3, and when c is 1, a4 may be 1.

In manufacturing the organic light emitting device of the present disclosure, when the heterocyclic compound represented by Chemical Formula 1 and the heterocyclic compound of Chemical Formula A are included in the organic material layer of the organic light emitting device, excellent effects of improving driving voltage, light emission efficiency and lifetime of the organic light emitting device are obtained.

This may lead to a forecast that an exciplex phenomenon occurs when the two heterocyclic compounds are mixed and included at the same time.

The exciplex phenomenon is a phenomenon of releasing energy different between a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and therefore, a driving voltage may be lowered, which resultantly helps with enhancement in the lifetime. In other words, when using the compound represented by Chemical Formula 1 as the acceptor and using the compound represented by Chemical Formula A as the donor, excellent properties of the organic light emitting device are obtained.

In one embodiment of the present disclosure, the heterocyclic compound represented by Chemical Formula A may be any one selected from the group consisting of the following compounds:

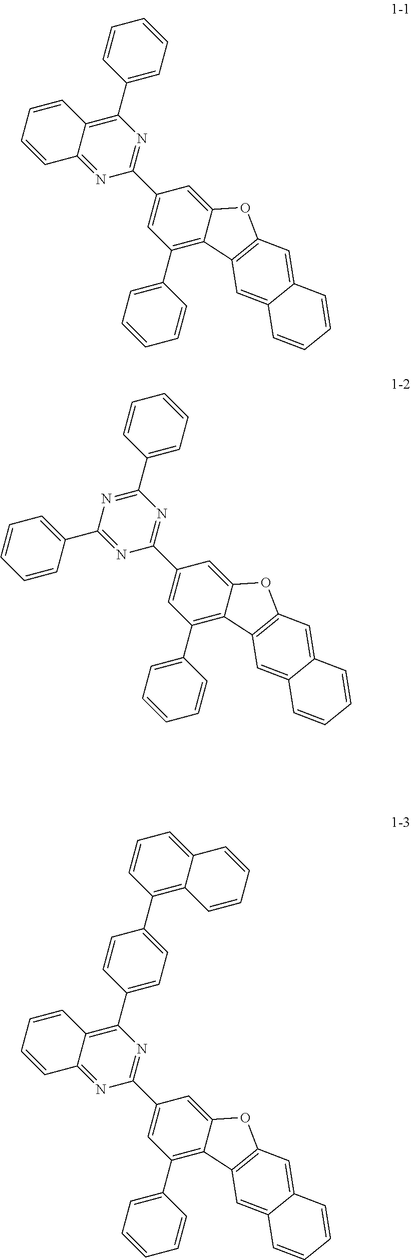 [SMILES: c1ccc(-c2nc(-c3cc(-c4ccccc4)c4c(c3)oc3cc5ccccc5cc34)nc3ccccc23)cc1 ; c1ccc(-c2nc(-c3ccccc3)nc(-c3cc(-c4ccccc4)c4c(c3)oc3cc5ccccc5cc34)n2)cc1 ; c1ccc(-c2cc(-c3nc(-c4ccc(-c5cccc6ccccc56)cc4)c4ccccc4n3)cc3oc4cc5ccccc5cc4c23)cc1]

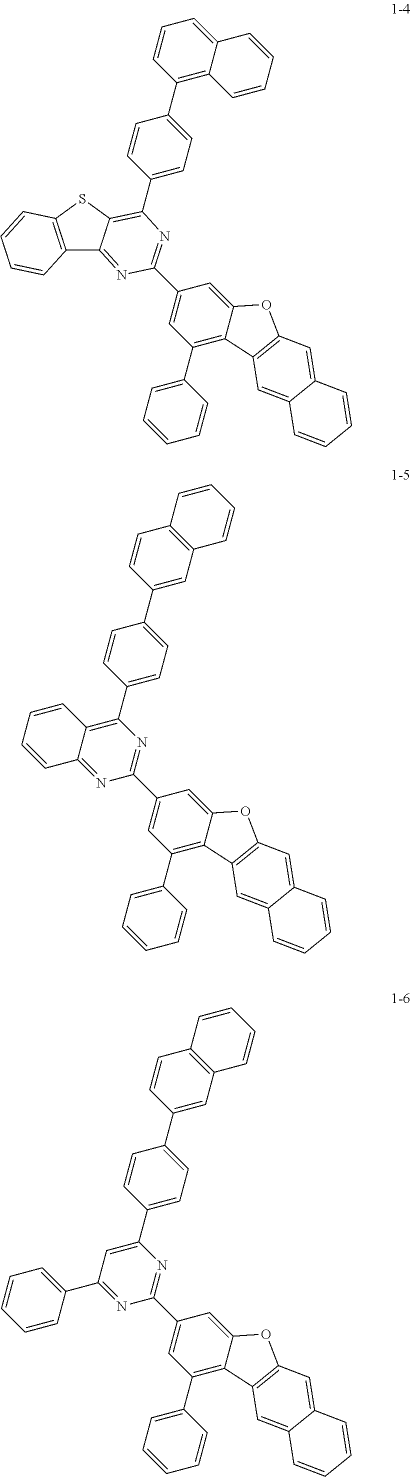 [SMILES: c1ccc(-c2cc(-c3nc(-c4ccc(-c5cccc6ccccc56)cc4)c4sc5ccccc5c4n3)cc3oc4cc5ccccc5cc4c23)cc1 ; c1ccc(-c2cc(-c3nc(-c4ccc(-c5ccc6ccccc6c5)cc4)c4ccccc4n3)cc3oc4cc5ccccc5cc4c23)cc1 ; c1ccc(-c2cc(-c3ccc(-c4ccc5ccccc5c4)cc3)nc(-c3cc(-c4ccccc4)c4c(c3)oc3cc5ccccc5cc34)n2)cc1]

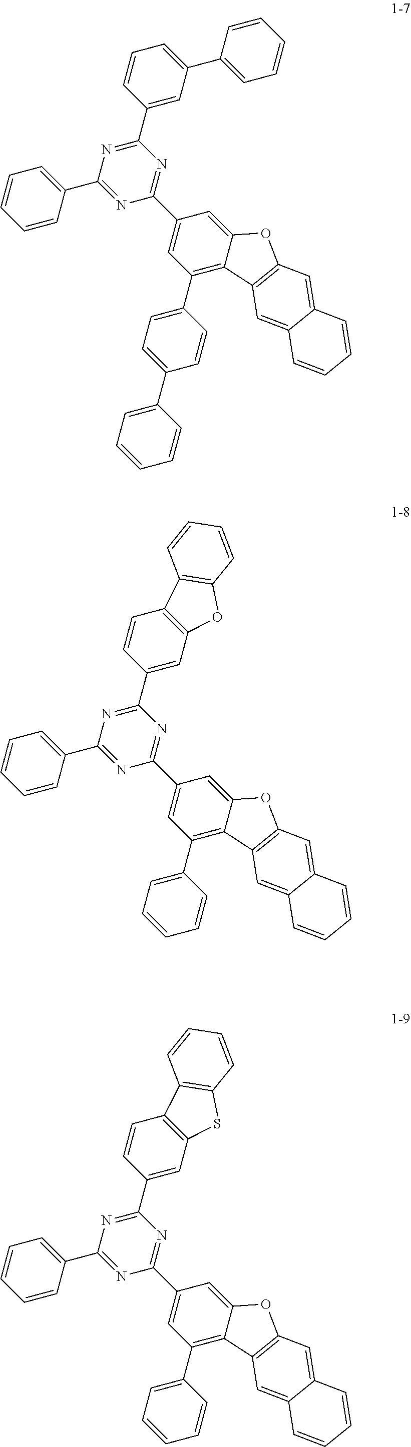 [SMILES: c1ccc(-c2ccc(-c3cc(-c4nc(-c5ccccc5)nc(-c5cccc(-c6ccccc6)c5)n4)cc4oc5cc6ccccc6cc5c34)cc2)cc1 ; c1ccc(-c2nc(-c3ccc4c(c3)oc3ccccc34)nc(-c3cc(-c4ccccc4)c4c(c3)oc3cc5ccccc5cc34)n2)cc1 ; c1ccc(-c2nc(-c3cc(-c4ccccc4)c4c(c3)oc3cc5ccccc5cc34)nc(-c3ccc4c(c3)sc3ccccc34)n2)cc1]

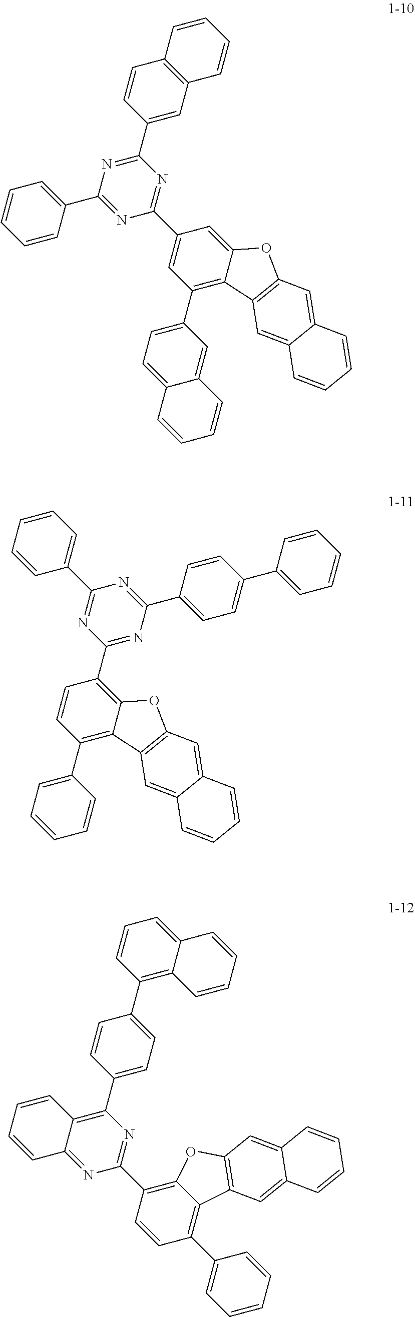 [SMILES: c1ccc(-c2nc(-c3ccc4ccccc4c3)nc(-c3cc(-c4ccc5ccccc5c4)c4c(c3)oc3cc5ccccc5cc34)n2)cc1 ; c1ccc(-c2ccc(-c3nc(-c4ccccc4)nc(-c4ccc(-c5ccccc5)c5c4oc4cc6ccccc6cc45)n3)cc2)cc1 ; c1ccc(-c2ccc(-c3nc(-c4ccc(-c5cccc6ccccc56)cc4)c4ccccc4n3)c3oc4cc5ccccc5cc4c23)cc1]

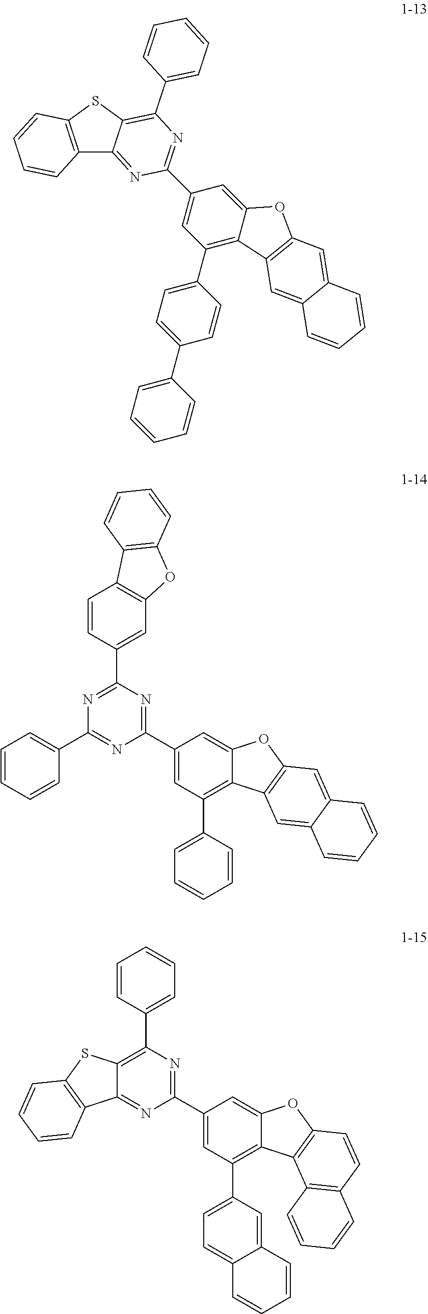 [SMILES: c1ccc(-c2ccc(-c3cc(-c4nc(-c5ccccc5)c5sc6ccccc6c5n4)cc4oc5cc6ccccc6cc5c34)cc2)cc1 ; c1ccc(-c2nc(-c3ccc4c(c3)oc3ccccc34)nc(-c3cc(-c4ccccc4)c4c(c3)oc3cc5ccccc5cc34)n2)cc1 ; c1ccc(-c2nc(-c3cc(-c4ccc5ccccc5c4)c4c(c3)oc3ccc5ccccc5c34)nc3c2sc2ccccc23)cc1]

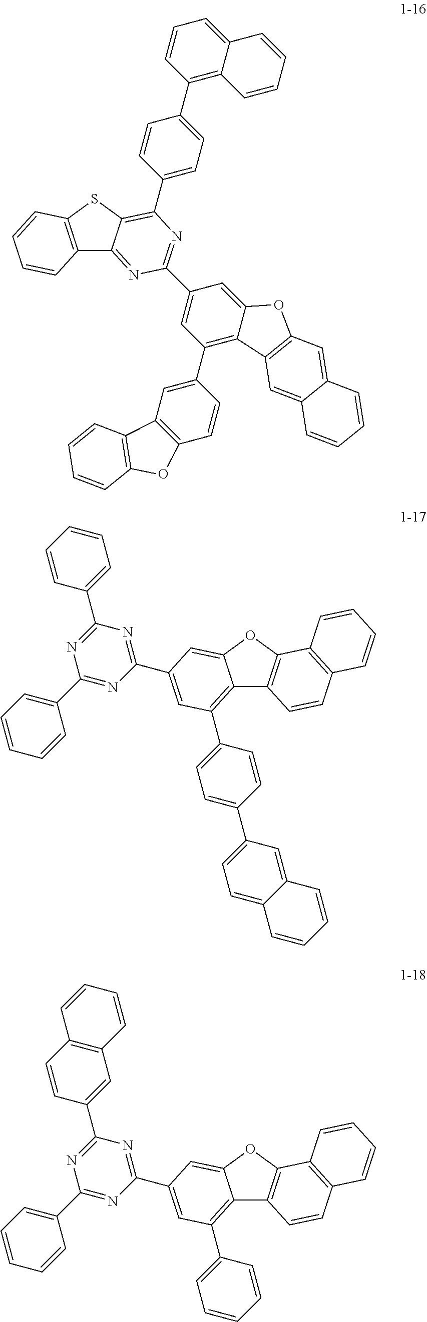

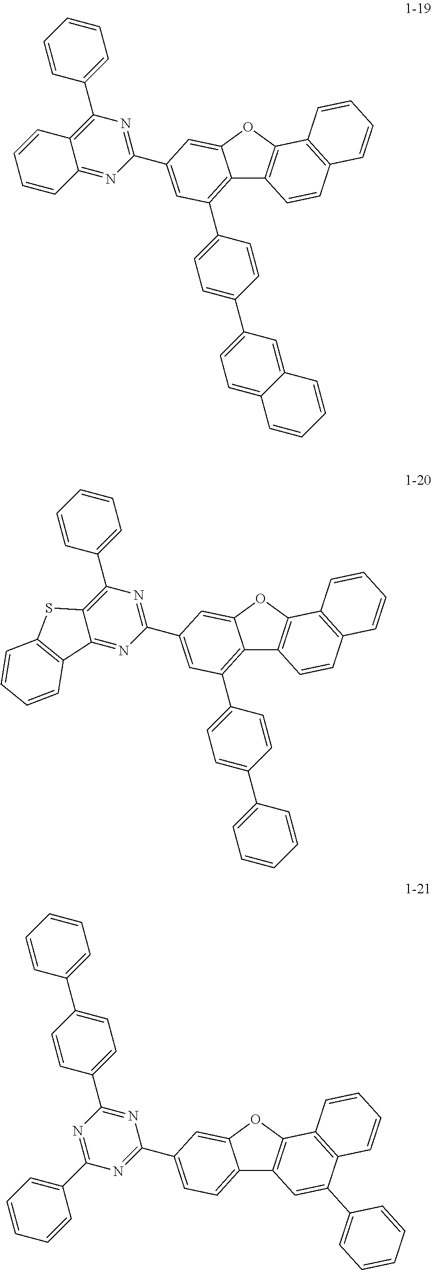 [SMILES: c1ccc(-c2nc(-c3cc(-c4ccc(-c5ccc6ccccc6c5)cc4)c4c(c3)oc3c5ccccc5ccc34)nc3ccccc23)cc1 ; c1ccc(-c2ccc(-c3cc(-c4nc(-c5ccccc5)c5sc6ccccc6c5n4)cc4oc5c6ccccc6ccc5c34)cc2)cc1 ; c1ccc(-c2ccc(-c3nc(-c4ccccc4)nc(-c4ccc5c(c4)oc4c6ccccc6c(-c6ccccc6)cc54)n3)cc2)cc1]

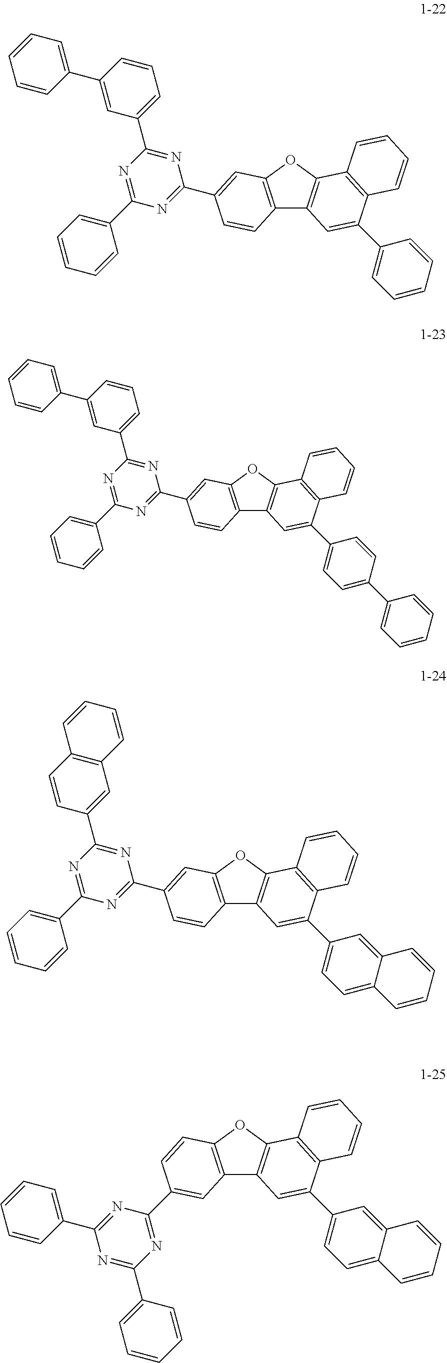 [SMILES: c1ccc(-c2cccc(-c3nc(-c4ccccc4)nc(-c4ccc5c(c4)oc4c6ccccc6c(-c6ccccc6)cc54)n3)c2)cc1 ; c1ccc(-c2ccc(-c3cc4c5ccc(-c6nc(-c7ccccc7)nc(-c7cccc(-c8ccccc8)c7)n6)cc5oc4c4ccccc34)cc2)cc1 ; c1ccc(-c2nc(-c3ccc4ccccc4c3)nc(-c3ccc4c(c3)oc3c5ccccc5c(-c5ccc6ccccc6c5)cc43)n2)cc1 ; c1ccc(-c2nc(-c3ccccc3)nc(-c3ccc4oc5c6ccccc6c(-c6ccc7ccccc7c6)cc5c4c3)n2)cc1]

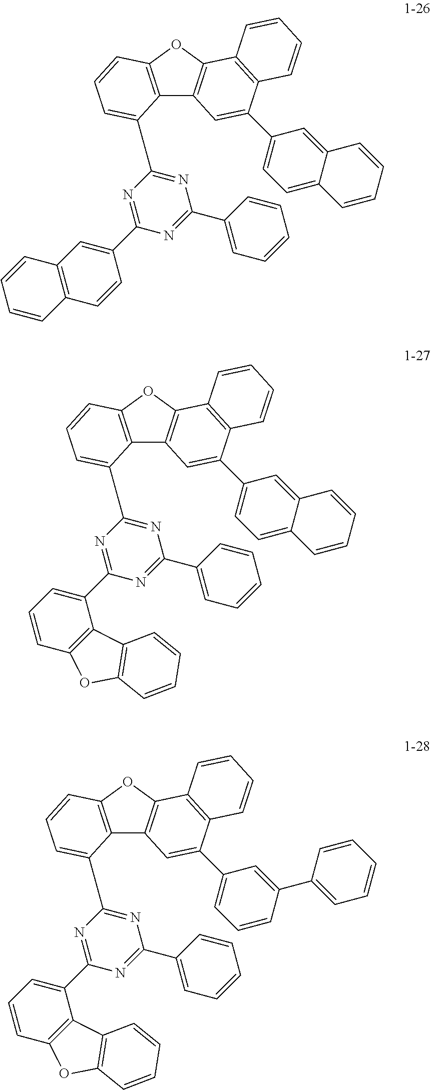 [SMILES: c1ccc(-c2nc(-c3ccc4ccccc4c3)nc(-c3cccc4oc5c6ccccc6c(-c6ccc7ccccc7c6)cc5c34)n2)cc1 ; c1ccc(-c2nc(-c3cccc4oc5ccccc5c34)nc(-c3cccc4oc5c6ccccc6c(-c6ccc7ccccc7c6)cc5c34)n2)cc1 ; c1ccc(-c2cccc(-c3cc4c(oc5cccc(-c6nc(-c7ccccc7)nc(-c7cccc8oc9ccccc9c78)n6)c54)c4ccccc34)c2)cc1]

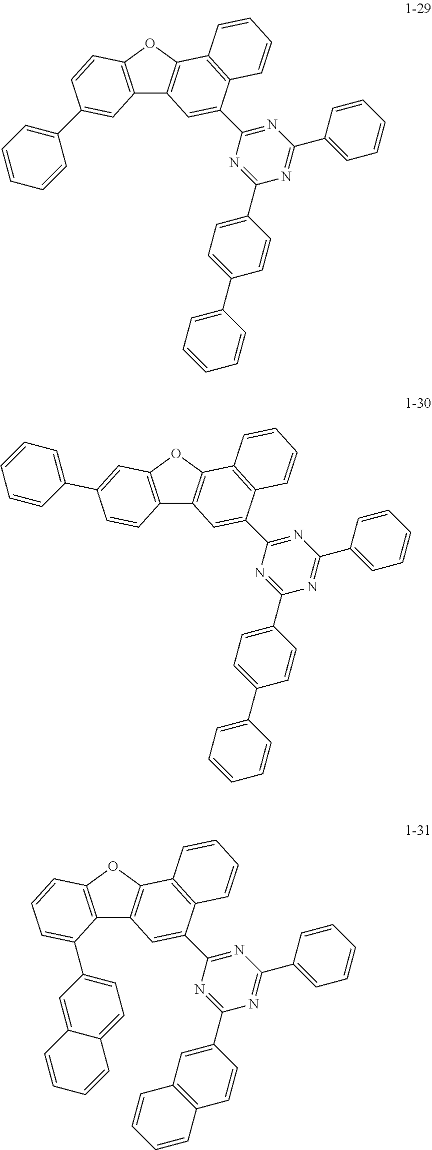 [SMILES: c1ccc(-c2ccc(-c3nc(-c4ccccc4)nc(-c4cc5c6cc(-c7ccccc7)ccc6oc5c5ccccc45)n3)cc2)cc1 ; c1ccc(-c2ccc(-c3nc(-c4ccccc4)nc(-c4cc5c6ccc(-c7ccccc7)cc6oc5c5ccccc45)n3)cc2)cc1 ; c1ccc(-c2nc(-c3ccc4ccccc4c3)nc(-c3cc4c(oc5cccc(-c6ccc7ccccc7c6)c54)c4ccccc34)n2)cc1]

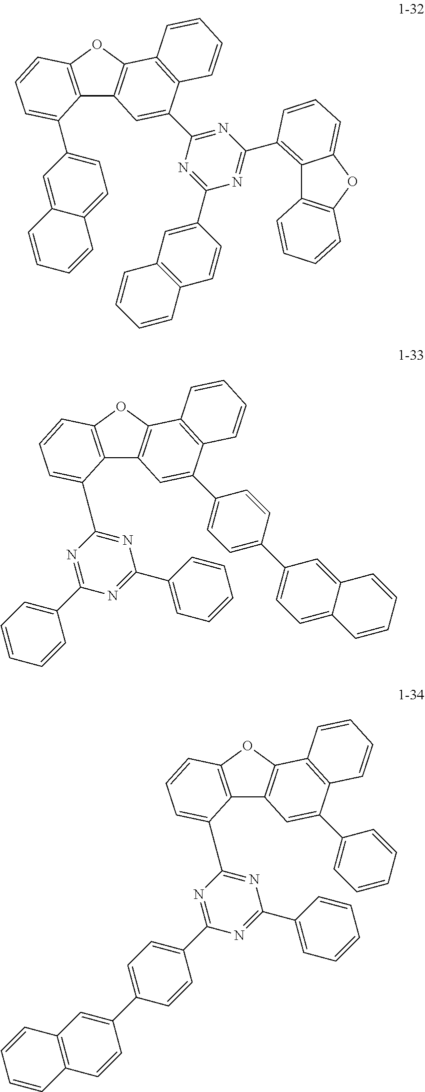 [SMILES: c1ccc2cc(-c3nc(-c4cc5c(oc6cccc(-c7ccc8ccccc8c7)c65)c5ccccc45)nc(-c4cccc5oc6ccccc6c45)n3)ccc2c1 ; c1ccc(-c2nc(-c3ccccc3)nc(-c3cccc4oc5c6ccccc6c(-c6ccc(-c7ccc8ccccc8c7)cc6)cc5c34)n2)cc1 ; c1ccc(-c2nc(-c3ccc(-c4ccc5ccccc5c4)cc3)nc(-c3cccc4oc5c6ccccc6c(-c6ccccc6)cc5c34)n2)cc1]

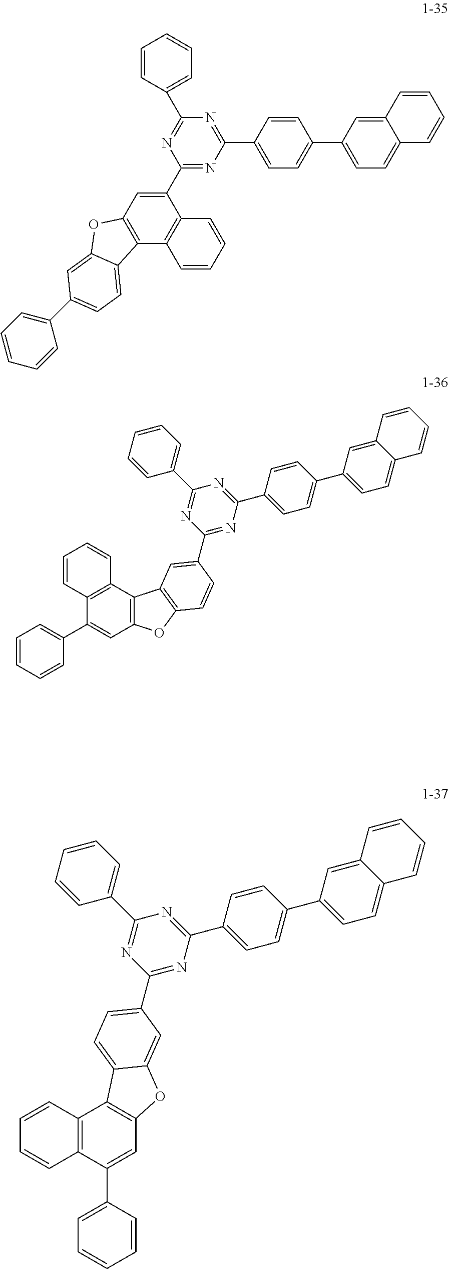 [SMILES: c1ccc(-c2ccc3c(c2)oc2cc(-c4nc(-c5ccccc5)nc(-c5ccc(-c6ccc7ccccc7c6)cc5)n4)c4ccccc4c23)cc1 ; c1ccc(-c2nc(-c3ccc(-c4ccc5ccccc5c4)cc3)nc(-c3ccc4oc5cc(-c6ccccc6)c6ccccc6c5c4c3)n2)cc1 ; c1ccc(-c2nc(-c3ccc(-c4ccc5ccccc5c4)cc3)nc(-c3ccc4c(c3)oc3cc(-c5ccccc5)c5ccccc5c34)n2)cc1]

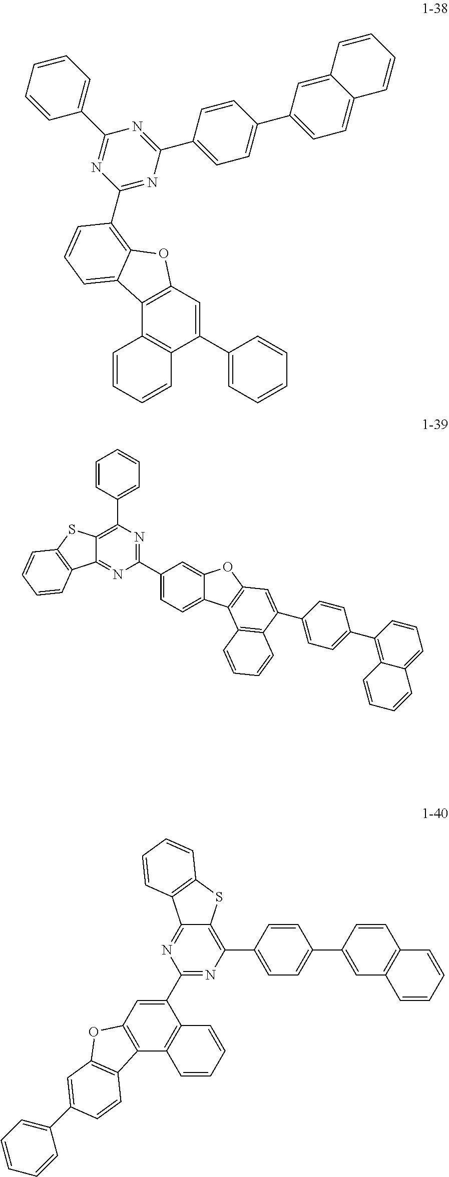 [SMILES: c1ccc(-c2nc(-c3ccc(-c4ccc5ccccc5c4)cc3)nc(-c3cccc4c3oc3cc(-c5ccccc5)c5ccccc5c34)n2)cc1 ; c1ccc(-c2nc(-c3ccc4c(c3)oc3cc(-c5ccc(-c6cccc7ccccc67)cc5)c5ccccc5c34)nc3c2sc2ccccc23)cc1 ; c1ccc(-c2ccc3c(c2)oc2cc(-c4nc(-c5ccc(-c6ccc7ccccc7c6)cc5)c5sc6ccccc6c5n4)c4ccccc4c23)cc1]

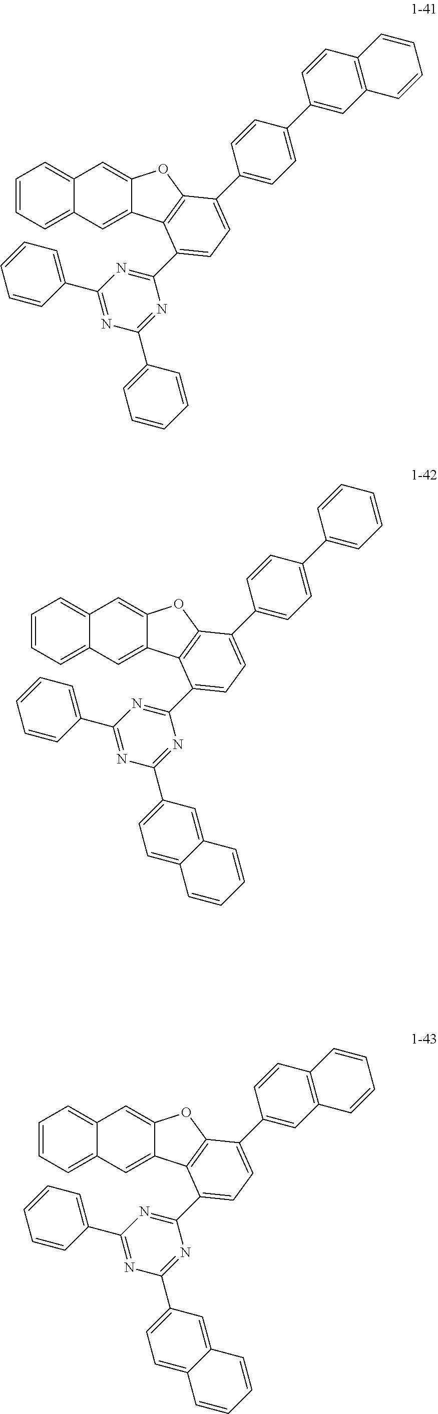 [SMILES: c1ccc(-c2nc(-c3ccccc3)nc(-c3ccc(-c4ccc(-c5ccc6ccccc6c5)cc4)c4oc5cc6ccccc6cc5c34)n2)cc1 ; c1ccc(-c2ccc(-c3ccc(-c4nc(-c5ccccc5)nc(-c5ccc6ccccc6c5)n4)c4c3oc3cc5ccccc5cc34)cc2)cc1 ; c1ccc(-c2nc(-c3ccc4ccccc4c3)nc(-c3ccc(-c4ccc5ccccc5c4)c4oc5cc6ccccc6cc5c34)n2)cc1]

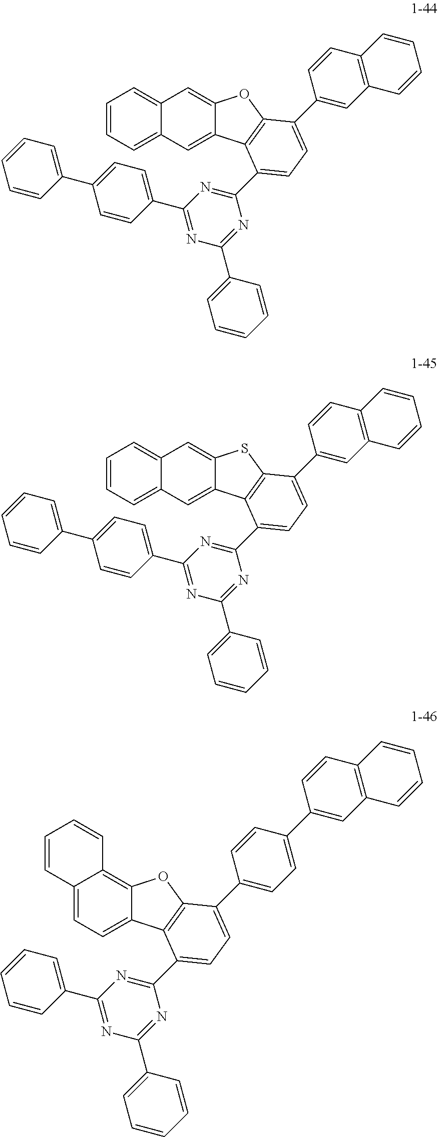 [SMILES: c1ccc(-c2ccc(-c3nc(-c4ccccc4)nc(-c4ccc(-c5ccc6ccccc6c5)c5oc6cc7ccccc7cc6c45)n3)cc2)cc1 ; c1ccc(-c2ccc(-c3nc(-c4ccccc4)nc(-c4ccc(-c5ccc6ccccc6c5)c5sc6cc7ccccc7cc6c45)n3)cc2)cc1 ; c1ccc(-c2nc(-c3ccccc3)nc(-c3ccc(-c4ccc(-c5ccc6ccccc6c5)cc4)c4oc5c6ccccc6ccc5c34)n2)cc1]

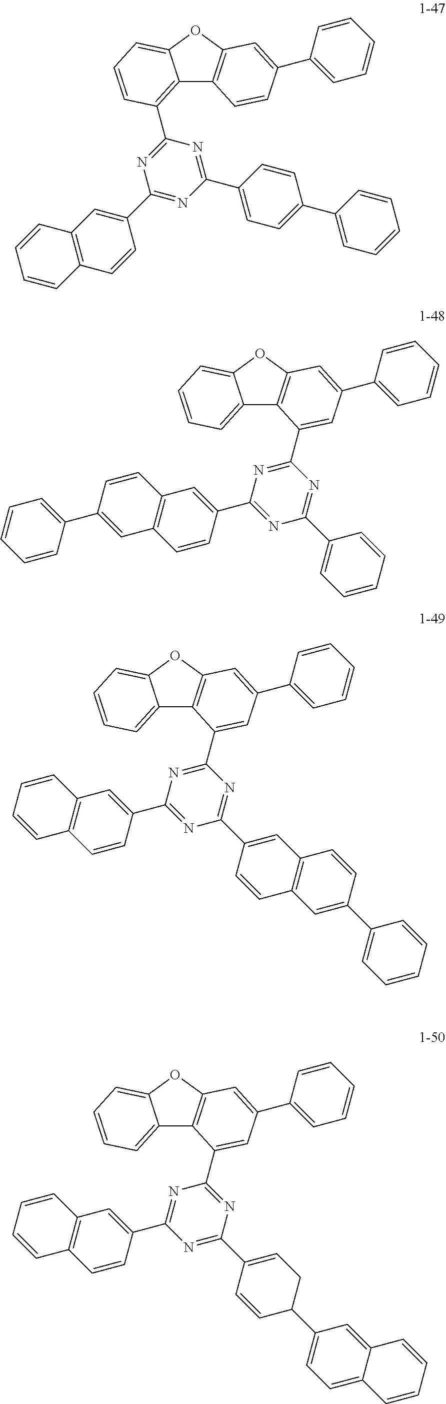 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc3cc(-c4nc(-c5ccccc5)nc(-c5cc(-c6ccccc6)cc6oc7ccccc7c56)n4)ccc3c2)cc1 ; c1ccc(-c2ccc3cc(-c4nc(-c5ccc6ccccc6c5)nc(-c5cc(-c6ccccc6)cc6oc7ccccc7c56)n4)ccc3c2)cc1 ; C1=CC(c2ccc3ccccc3c2)CC=C1c1nc(-c2ccc3ccccc3c2)nc(-c2cc(-c3ccccc3)cc3oc4ccccc4c23)n1]

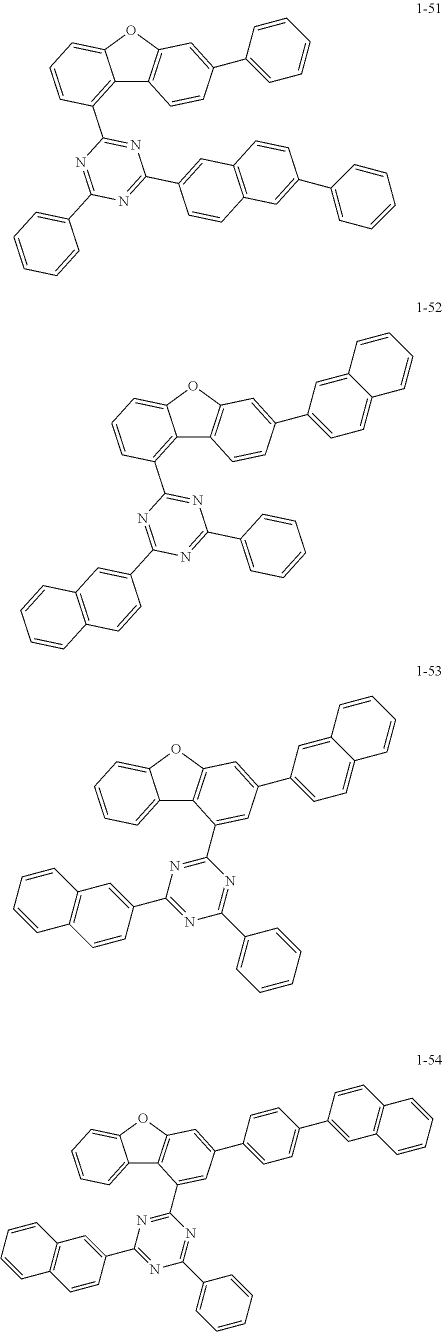 [SMILES: c1ccc(-c2ccc3cc(-c4nc(-c5ccccc5)nc(-c5cccc6oc7cc(-c8ccccc8)ccc7c56)n4)ccc3c2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3ccc4ccccc4c3)nc(-c3cc(-c4ccc(-c5ccc6ccccc6c5)cc4)cc4oc5ccccc5c34)n2)cc1]

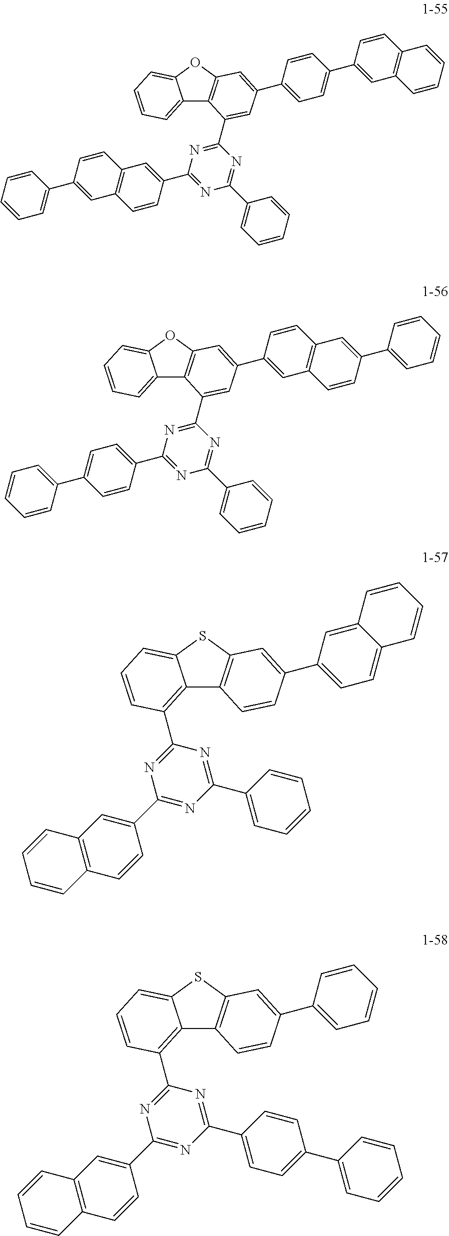 [SMILES: c1ccc(-c2ccc3cc(-c4nc(-c5ccccc5)nc(-c5cc(-c6ccc(-c7ccc8ccccc8c7)cc6)cc6oc7ccccc7c56)n4)ccc3c2)cc1 ; c1ccc(-c2ccc(-c3nc(-c4ccccc4)nc(-c4cc(-c5ccc6cc(-c7ccccc7)ccc6c5)cc5oc6ccccc6c45)n3)cc2)cc1 ; c1ccc(-c2nc(-c3ccc4ccccc4c3)nc(-c3cccc4sc5cc(-c6ccc7ccccc7c6)ccc5c34)n2)cc1 ; c1ccc(-c2ccc(-c3nc(-c4ccc5ccccc5c4)nc(-c4cccc5sc6cc(-c7ccccc7)ccc6c45)n3)cc2)cc1]

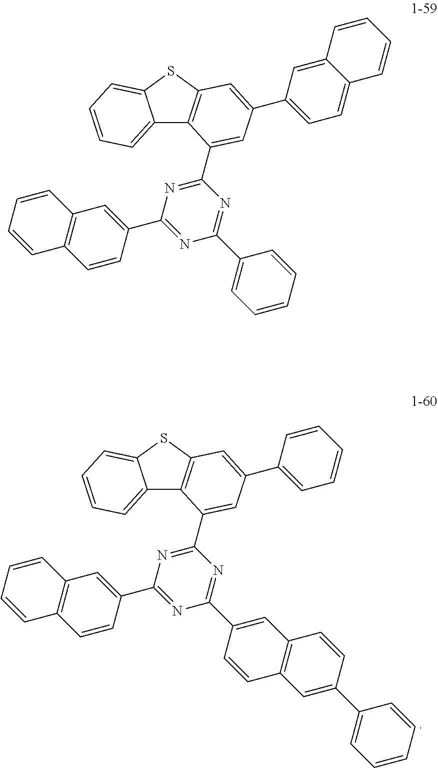 [SMILES: c1ccc(-c2nc(-c3ccc4ccccc4c3)nc(-c3cc(-c4ccc5ccccc5c4)cc4sc5ccccc5c34)n2)cc1 ; c1ccc(-c2ccc3cc(-c4nc(-c5ccc6ccccc6c5)nc(-c5cc(-c6ccccc6)cc6sc7ccccc7c56)n4)ccc3c2)cc1]

In addition, another embodiment of the present application provides a composition for an organic material layer of an organic light emitting device, the composition including: the heterocyclic compound represented by Chemical Formula 1; and the heterocyclic compound represented by Chemical Formula A.

Specific descriptions heterocyclic compound represented by Chemical Formula 1 and the heterocyclic compound represented by Chemical Formula A are the same as the descriptions provided above.

The heterocyclic compound represented by Chemical Formula 1 and the heterocyclic compound represented by Chemical Formula A may have a weight ratio of 1:10 to 10:1, 1:8 to 8:1, 1:6 to 6:1, 1:4 to 4:1, 1:3 to 3:1, 1:2 to 2:1, or 1:1 in the composition, however, the ratio is not limited thereto.

The composition may be used when forming an organic material of an organic light emitting device, and particularly, may be more preferably used when forming a host of a light emitting layer.

The composition has a form in which two or more compounds are simply mixed, and materials in a powder state may be mixed before forming an organic material layer of an organic light emitting device, or compounds in a liquid state at a proper temperature or higher may be mixed. The composition is in a solid state at a melting point of each material or lower, and may be kept in a liquid state when adjusting a temperature.

The composition may further include materials known in the art such as solvents and additives.

The organic light emitting device according to one embodiment of the present application may be manufactured using common organic light emitting device manufacturing methods and materials except that one or more organic material layers are formed using the heterocyclic compound represented by Chemical Formula 1 and the heterocyclic compound represented by Chemical Formula A described above.

The compound represented by Chemical Formula 1 and the heterocyclic compound represented by Chemical Formula A may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron transport layer, an electron injection layer and the like as the organic material layer. However, the structure of the organic light emitting device is not limited thereto, and may include a smaller number of organic material layers.

In one embodiment of the present application, the organic light emitting device may be a blue organic light emitting device, and the heterocyclic compound represented by Chemical Formula 1 and the heterocyclic compound according to Chemical Formula A may be used as a material of the blue organic light emitting device. In one embodiment of the present application, the organic light emitting device may be a green organic light emitting device, and the heterocyclic compound represented by Chemical Formula 1 and the heterocyclic compound represented by Chemical Formula A may be used as a material of the green organic light emitting device.

In one embodiment of the present application, the organic light emitting device may be a red organic light emitting device, and the heterocyclic compound represented by Chemical Formula 1 and the heterocyclic compound represented by Chemical Formula A may be used as a material of the red organic light emitting device.

The organic light emitting device of the present disclosure may further include one, two or more layers selected from the group consisting of a light emitting layer, a hole injection layer, a hole transport layer, an electron injection layer, an electron transport layer, an electron blocking layer and a hole blocking layer.

One embodiment of the present application provides an organic light emitting device, wherein the organic material layer includes at least one of a hole blocking layer, an electron injection layer and an electron transport layer, and at least one of the hole blocking layer, the electron injection layer and the electron transport layer includes the heterocyclic compound represented by Chemical Formula 1 and the heterocyclic compound represented by Chemical Formula A.

One embodiment of the present application provides an organic light emitting device, wherein the organic material layer includes a light emitting layer, and the light emitting layer includes the heterocyclic compound represented by Chemical Formula 1 and the heterocyclic compound represented by Chemical Formula A.

One embodiment of the present application provides an organic light emitting device, wherein the organic material layer includes a light emitting layer, the light emitting layer includes a host material, and the host material includes the heterocyclic compound represented by Chemical Formula 1 and the heterocyclic compound represented by Chemical Formula A.

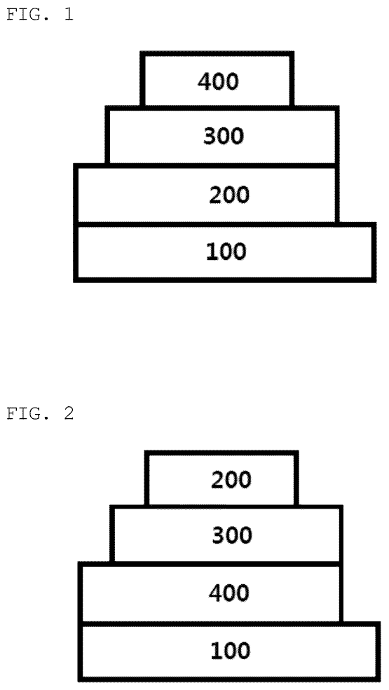

FIGS. 1 to 3 illustrate a lamination order of electrodes and organic material layers of the organic light emitting device according to one embodiment of the present disclosure. However, it is not intended that the scope of the present application be limited by these drawings, and structures of organic light emitting devices known in the art may also be applied to the present application.

FIG. 1 illustrates an organic light emitting device in which a positive electrode 200, an organic material layer 300 and a negative electrode 400 are sequentially laminated on a substrate 100. However, the structure is not limited only to such a structure, and as illustrated in FIG. 2, an organic light emitting device in which a negative electrode, an organic material layer and a positive electrode are sequentially laminated on a substrate may also be obtained.

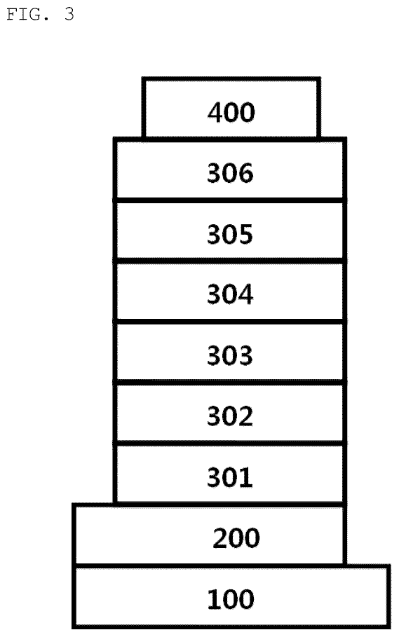

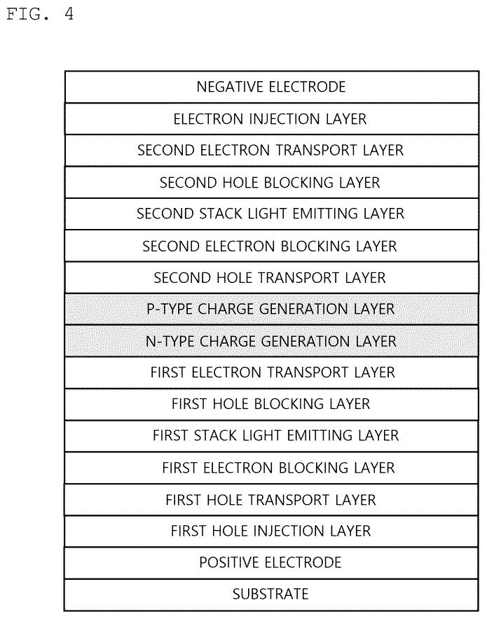

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

As the organic light emitting device according to one embodiment of the present application, an organic light emitting device having a 2-stack tandem structure is schematically illustrated in FIG. 4.

Herein, a first electron blocking layer, a first hole blocking layer, a second hole blocking layer and the like described in FIG. 4 may not be included in some cases.

One embodiment of the present disclosure provides a method for manufacturing an organic light emitting device, the method including: preparing a substrate; forming a first electrode on the substrate; forming one or more organic material layers on the first electrode; and forming a second electrode on the organic material layers, wherein the forming of organic material layers includes forming one or more organic material layers using the composition for an organic material layer according to one embodiment of the present disclosure.

In one embodiment of the present disclosure, the forming of organic material layers may be forming organic material layers using a thermal vacuum deposition method after pre-mixing the heterocyclic compound represented by Chemical Formula 1.

The pre-mixing means, before depositing the heterocyclic compound represented by Chemical Formula 1 on the organic material layer, putting and mixing the materials first in one source of supply.

The pre-mixed material may be referred to as the composition for an organic material layer according to one embodiment of the present application.

The organic material layer including the heterocyclic compound represented by Chemical Formula 1 may further include other materials as necessary.

In the organic light emitting device according to one embodiment of the present disclosure, materials other than the heterocyclic compound represented by Chemical Formula 1 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as Zno:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature[Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### PREPARATION EXAMPLE

### Preparation Example 1: Preparation of Target Compounds

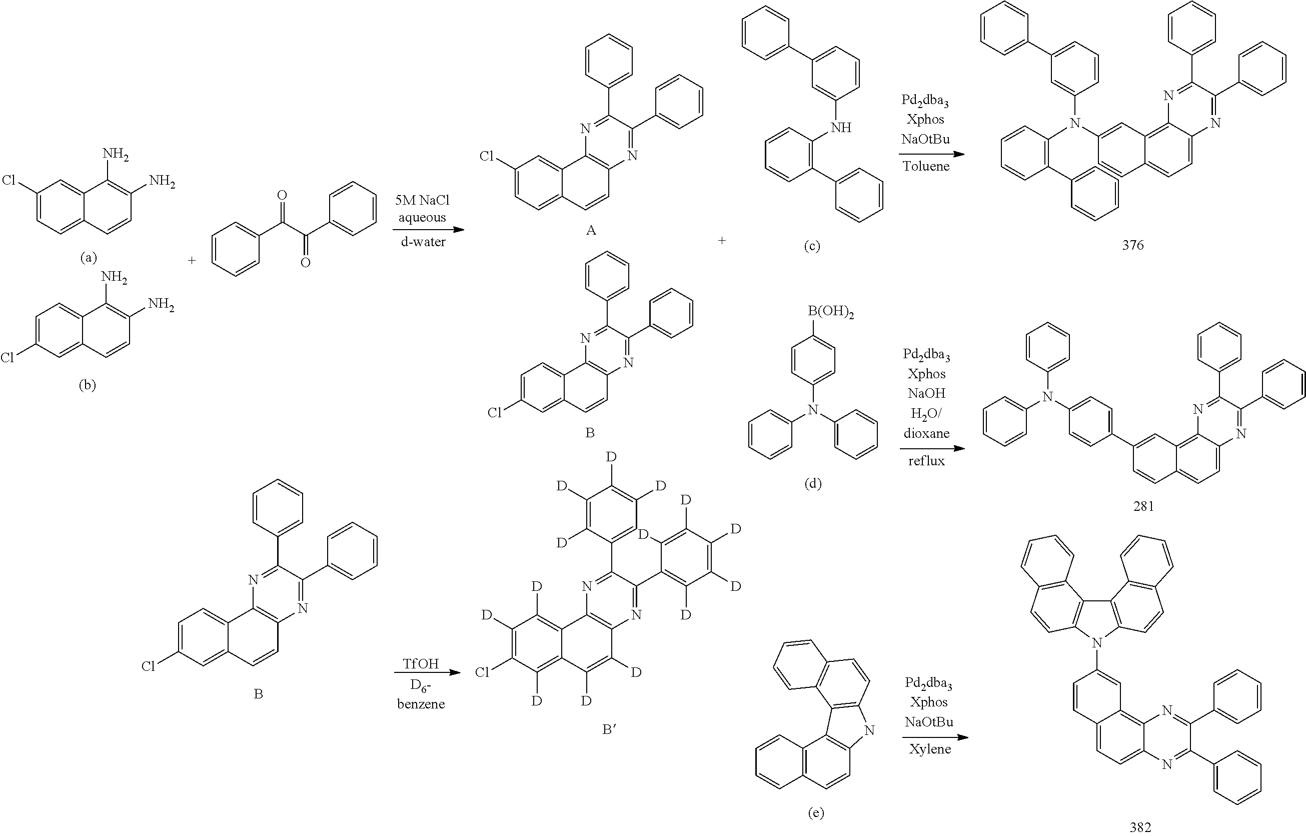

Using 7-chloronaphthalene-1,2-diamine that is Intermediate Compound (a) or 6-chloronaphthalene-1,2-diamine that is Intermediate Compound (b), 9-chloro-2,3-diphenylbenzo[f]quinoxaline that is Compound A or 8-chloro-2,3-diphenylbenzo[f]quinoxaline that is Compound B was prepared, respectively.

1) Preparation of Compound A

7-chloronaphthalene-1,2-diamine (10.0 g, 51.90 mmol) and benzil (10.91 g, 51.90 mmol) were introduced to a 5 M aqueous NaCl solution (100 ml) and dissolved therein, and then the mixture was refluxed for 2 hours at 95° C. After the reaction was completed, the result was extracted by introducing distilled water and dichloromethane (DCM) thereto at room temperature. The organic layer was dried with magnesium sulfate (MgSO₄), and then the solvent was removed using a rotary evaporator. The reaction material was purified by column chromatography (DCM:Hex=1:5) to obtain Compound A (17.25 g, yield 90.61%).

2) Preparation of Compound B′ (substituted with deuterium in Intermediate B of Compound 359 or Compound 478)

Compound B (5.0 g, 13.62 mmol) and benzene-d6 (50 mL) were introduced to a 500 mL round bottom flask, and trifluoromethanesulfonic acid (triflic acid, TfOH) (16.84 mL) was slowly added dropwise thereto. After that, the mixture was stirred for 1 hour at 100° C. After the reaction was completed, the result was cooled to room temperature, and washed with excess distilled water and methanol. After that, the result was dried, and then separated by a column under a condition of MC:Hex=1:2 to obtain Compound B′ (4.9 g, 11.9 mmol, yield 94.5%).
3) Preparation of Compound 376

Compound A (5 g, 13.62 mmol), N-([1,1′-biphenyl]-3-yl)-[1,1′-biphenyl]-2-amine (4.3 mmol), g, 13.62 mmol), tris(dibenzylideneacetone) dipalladium (Pd₂(dba)₃) (0.62 g, 0.68 mmol), XPhos (0.65 g, 1.36 mmol) and sodium tert-butoxide (t-BuONa) (2.62 g, 27.24 mmol) were introduced to toluene (50 ml) and dissolved therein, and then the mixture was refluxed for 6 hours at 100° C. After the reaction was completed, produced solid was filtered, washed with methanol (MeOH) and dried. The dried solid was dissolved in chloroform and purified by silica, and then the solvent was removed using a rotary evaporator. The result was recrystallized with acetone to obtain target Compound 376 (7.0 g, yield 78.92%).

4) Preparation of Compound 281

Compound A (5 g, 13.62 mmol), (4-(diphenylamino)phenyl) boronic acid (3.94 g, 13.62 mmol), tris(dibenzylideneacetone) dipalladium (Pd₂(dba)₃) (0.62 g, 0.68 mmol), Xphos (0.65 g, 1.36 mmol) and sodium hydroxide (NaOH) (1.1 g, 27.24 mmol) were introduced to 1,4-dioxane (50 ml) and water (H₂O) (10 ml) and dissolved therein, and then the mixture was refluxed for 4 hours at 120° C. After the reaction was completed, produced solid was filtered, washed with methanol (MeOH) and dried. The dried solid was dissolved in chloroform and purified by silica, and then the solvent was removed using a rotary evaporator. The result was recrystallized with acetone to obtain target Compound 281 (6.2 g, yield 79.08%).

5) Preparation of Compound 382

Compound A (5 g, 13.62 mmol), 7-dibenzo[c, g]carbazole (3.6 g, 13.62 mmol), tris(dibenzylideneacetone) dipalladium (Pd₂ (dba)₃) (0.62 g, 0.68 mmol), Xphos (0.65 g, 1.36 mmol) and sodium tert-butoxide (t-BuONa) (2.62 g, 27.24 mmol) were introduced to xylene (50 ml) and dissolved therein, and then the mixture was refluxed for 5 hours at 150° C. After the reaction was completed, produced solid was filtered, washed with methanol (MeOH) and dried. The dried solid was dissolved in chloroform and purified by silica, and then the solvent was removed using a rotary evaporator. The result was recrystallized with acetone to obtain target Compound 382 (5.8 g, yield 71.25).

Target compounds were synthesized in the same manner as in Preparation Example 1, using Intermediates (c), (d) and (e) of the following Table 1 instead of Intermediate Compounds (c), (d) and (e) in Preparation Example 1.

TABLE 1

| Com- | | | | |
|---|---|---|---|---|
| pound | Intermediate | Intermediate | | |
| No. | (a) / (b) | (c) / (d) / (e) | Target Compound | Yield |
| 281 | 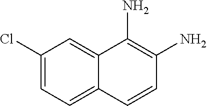 [SMILES: Nc1ccc2ccc(Cl)cc2c1N] | 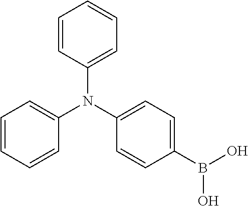 [SMILES: OB(O)c1ccc(N(c2ccccc2)c2ccccc2)cc1] | 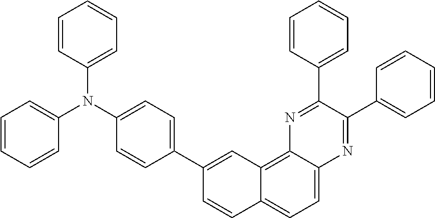 [SMILES: c1ccc(-c2nc3ccc4ccc(-c5ccc(N(c6ccccc6)c6ccccc6)cc5)cc4c3nc2-c2ccccc2)cc1] | 72% |
| 287 | 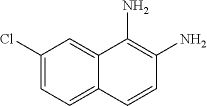 [SMILES: Nc1ccc2ccc(Cl)cc2c1N] | 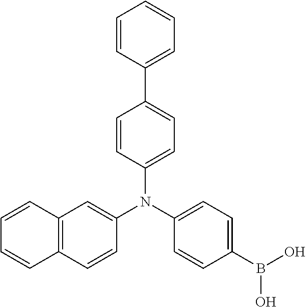 [SMILES: OB(O)c1ccc(N(c2ccc(-c3ccccc3)cc2)c2ccc3ccccc3c2)cc1] | 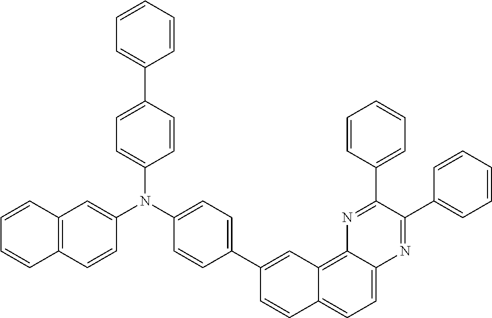 [SMILES: c1ccc(-c2ccc(N(c3ccc(-c4ccc5ccc6nc(-c7ccccc7)c(-c7ccccc7)nc6c5c4)cc3)c3ccc4ccccc4c3)cc2)cc1] | 70% |
| 297 | 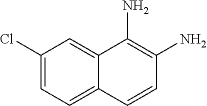 [SMILES: Nc1ccc2ccc(Cl)cc2c1N] | 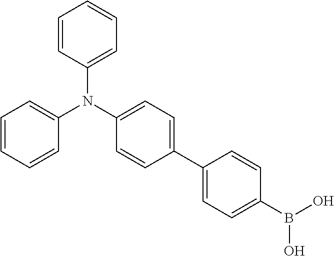 [SMILES: OB(O)c1ccc(-c2ccc(N(c3ccccc3)c3ccccc3)cc2)cc1] | 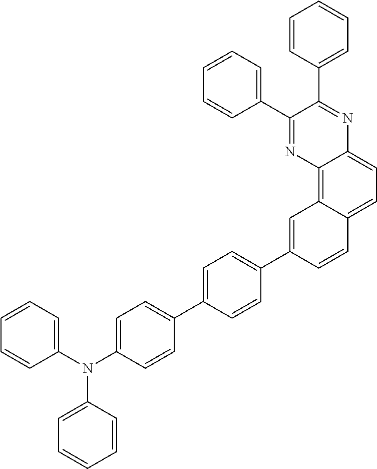 [SMILES: c1ccc(-c2nc3ccc4ccc(-c5ccc(-c6ccc(N(c7ccccc7)c7ccccc7)cc6)cc5)cc4c3nc2-c2ccccc2)cc1] | 76% |
| 305 | 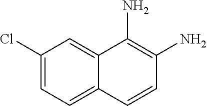 [SMILES: Nc1ccc2ccc(Cl)cc2c1N] | 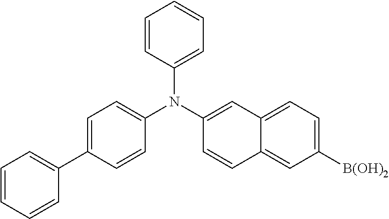 [SMILES: OB(O)c1ccc2cc(N(c3ccccc3)c3ccc(-c4ccccc4)cc3)ccc2c1] | 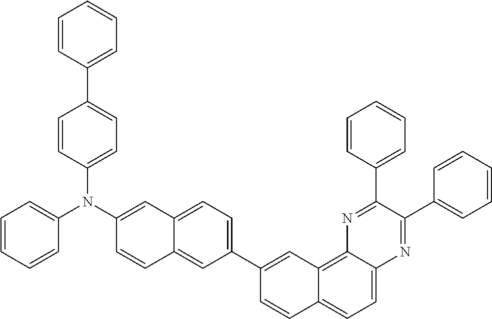 [SMILES: c1ccc(-c2ccc(N(c3ccccc3)c3ccc4cc(-c5ccc6ccc7nc(-c8ccccc8)c(-c8ccccc8)nc7c6c5)ccc4c3)cc2)cc1] | 68% |
| 313 | 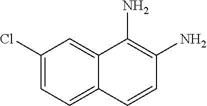 [SMILES: Nc1ccc2ccc(Cl)cc2c1N] | 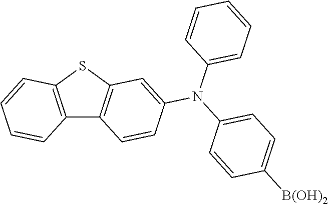 [SMILES: OB(O)c1ccc(N(c2ccccc2)c2ccc3c(c2)sc2ccccc23)cc1] | 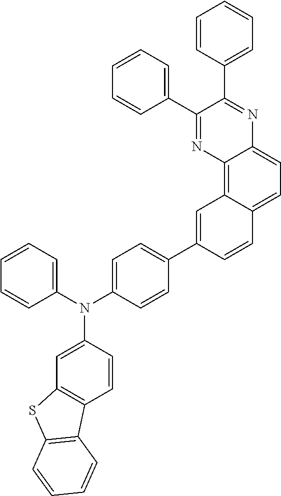 [SMILES: c1ccc(-c2nc3ccc4ccc(-c5ccc(N(c6ccccc6)c6ccc7c(c6)sc6ccccc67)cc5)cc4c3nc2-c2ccccc2)cc1] | 82% |
| 323 | 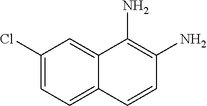 [SMILES: Nc1ccc2ccc(Cl)cc2c1N] | 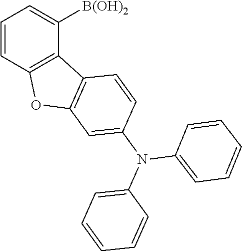 [SMILES: OB(O)c1cccc2oc3cc(N(c4ccccc4)c4ccccc4)ccc3c12] | 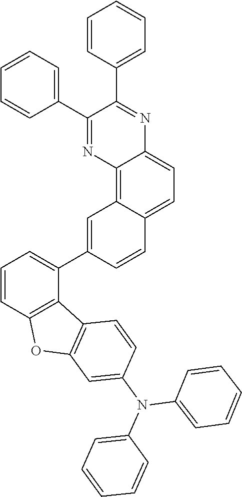 [SMILES: c1ccc(-c2nc3ccc4ccc(-c5cccc6oc7cc(N(c8ccccc8)c8ccccc8)ccc7c56)cc4c3nc2-c2ccccc2)cc1] | 75% |
| 336 | 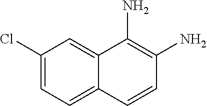 [SMILES: Nc1ccc2ccc(Cl)cc2c1N] | 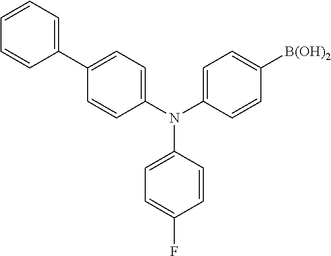 [SMILES: OB(O)c1ccc(N(c2ccc(F)cc2)c2ccc(-c3ccccc3)cc2)cc1] | 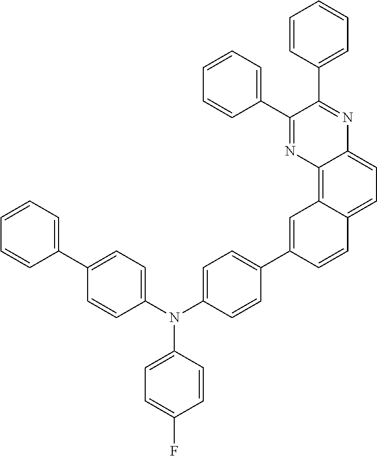 [SMILES: Fc1ccc(N(c2ccc(-c3ccccc3)cc2)c2ccc(-c3ccc4ccc5nc(-c6ccccc6)c(-c6ccccc6)nc5c4c3)cc2)cc1] | 70% |
| 338 | 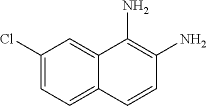 [SMILES: Nc1ccc2ccc(Cl)cc2c1N] | 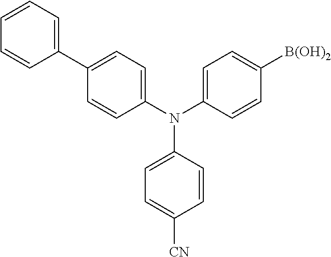 [SMILES: N#Cc1ccc(N(c2ccc(B(O)O)cc2)c2ccc(-c3ccccc3)cc2)cc1] | 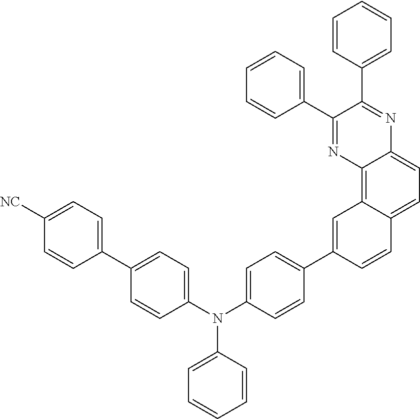 [SMILES: N#Cc1ccc(-c2ccc(N(c3ccccc3)c3ccc(-c4ccc5ccc6nc(-c7ccccc7)c(-c7ccccc7)nc6c5c4)cc3)cc2)cc1] | 71% |
| 340 | 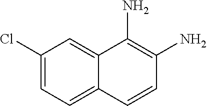 [SMILES: Nc1ccc2ccc(Cl)cc2c1N] | 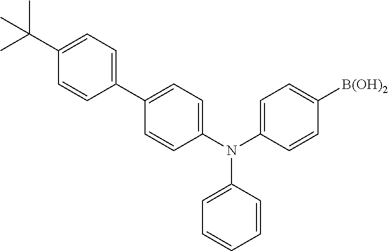 [SMILES: CC(C)(C)c1ccc(-c2ccc(N(c3ccccc3)c3ccc(B(O)O)cc3)cc2)cc1] | 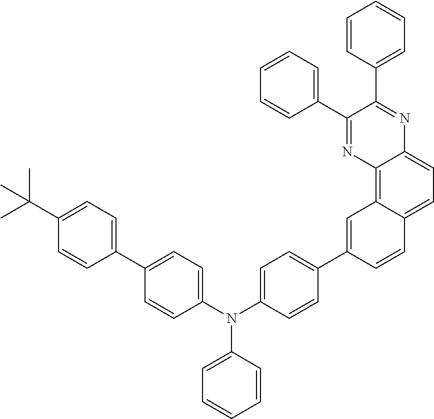 [SMILES: CC(C)(C)c1ccc(-c2ccc(N(c3ccccc3)c3ccc(-c4ccc5ccc6nc(-c7ccccc7)c(-c7ccccc7)nc6c5c4)cc3)cc2)cc1] | 79% |
| 356 | 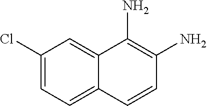 [SMILES: Nc1ccc2ccc(Cl)cc2c1N] | 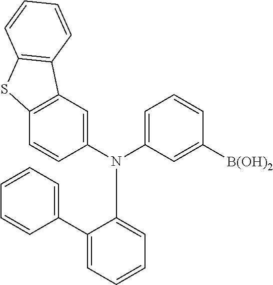 [SMILES: OB(O)c1cccc(N(c2ccc3sc4ccccc4c3c2)c2ccccc2-c2ccccc2)c1] | 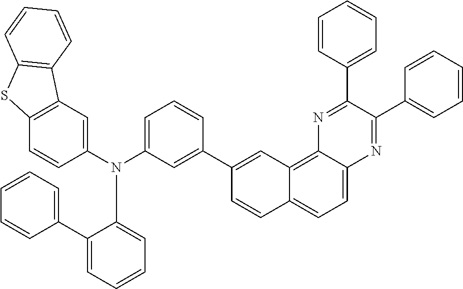 [SMILES: c1ccc(-c2ccccc2N(c2cccc(-c3ccc4ccc5nc(-c6ccccc6)c(-c6ccccc6)nc5c4c3)c2)c2ccc3sc4ccccc4c3c2)cc1] | 81% |
| 359 | 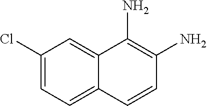 [SMILES: Nc1ccc2ccc(Cl)cc2c1N] | 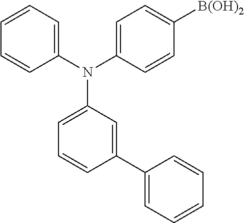 [SMILES: OB(O)c1ccc(N(c2ccccc2)c2cccc(-c3ccccc3)c2)cc1] | 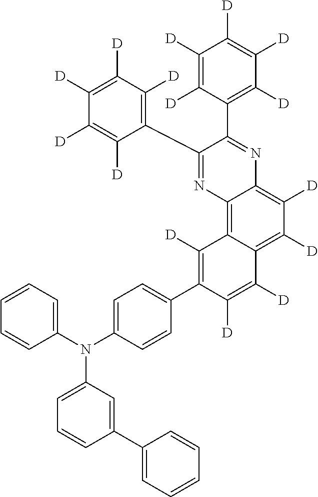 [SMILES: [2H]c1c([2H])c([2H])c(-c2nc3c([2H])c([2H])c4c([2H])c([2H])c(-c5ccc(N(c6ccccc6)c6cccc(-c7ccccc7)c6)cc5)c([2H])c4c3nc2-c2c([2H])c([2H])c([2H])c([2H])c2[2H])c([2H])c1[2H]] | 86% |
| 376 | 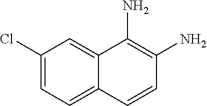 [SMILES: Nc1ccc2ccc(Cl)cc2c1N] | 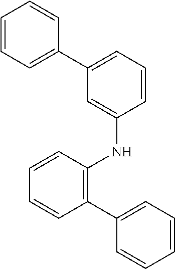 [SMILES: c1ccc(-c2cccc(Nc3ccccc3-c3ccccc3)c2)cc1] | 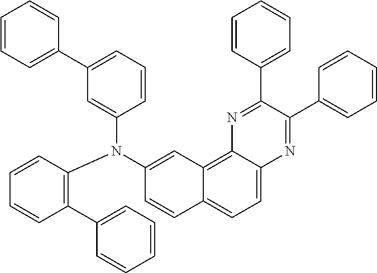 [SMILES: c1ccc(-c2cccc(N(c3ccc4ccc5nc(-c6ccccc6)c(-c6ccccc6)nc5c4c3)c3ccccc3-c3ccccc3)c2)cc1] | 66º |
| 382 | 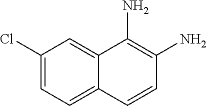 [SMILES: Nc1ccc2ccc(Cl)cc2c1N] | 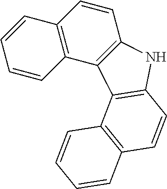 [SMILES: c1ccc2c(c1)ccc1[nH]c3ccc4ccccc4c3c12] | 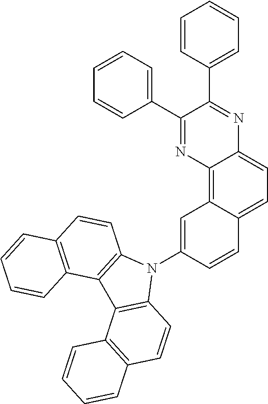 [SMILES: c1ccc(-c2nc3ccc4ccc(-n5c6ccc7ccccc7c6c6c7ccccc7ccc65)cc4c3nc2-c2ccccc2)cc1] | 69% |
| 391 | 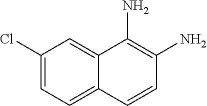 [SMILES: Nc1ccc2ccc(Cl)cc2c1N] | 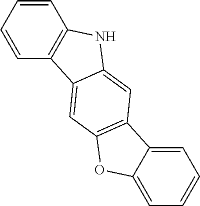 [SMILES: c1ccc2c(c1)[nH]c1cc3c(cc12)oc1ccccc13] | 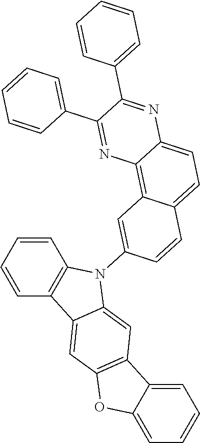 [SMILES: c1ccc(-c2nc3ccc4ccc(-n5c6ccccc6c6cc7oc8ccccc8c7cc65)cc4c3nc2-c2ccccc2)cc1] | 77% |
| 392 | 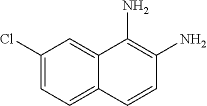 [SMILES: Nc1ccc2ccc(Cl)cc2c1N] | 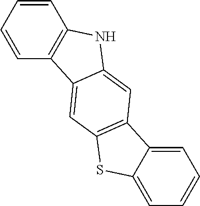 [SMILES: c1ccc2c(c1)[nH]c1cc3c(cc12)sc1ccccc13] | 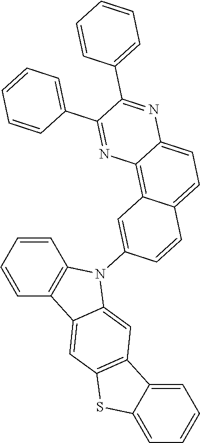 [SMILES: c1ccc(-c2nc3ccc4ccc(-n5c6ccccc6c6cc7sc8ccccc8c7cc65)cc4c3nc2-c2ccccc2)cc1] | 64% |
| 396 | 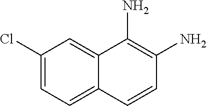 [SMILES: Nc1ccc2ccc(Cl)cc2c1N] | 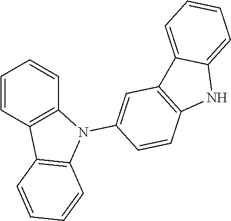 [SMILES: c1ccc2c(c1)[nH]c1ccc(-n3c4ccccc4c4ccccc43)cc12] | 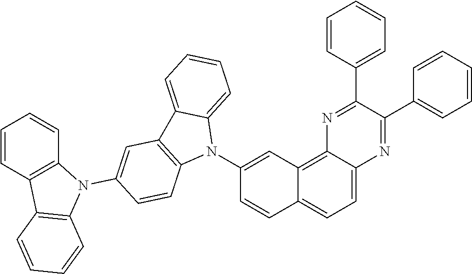 [SMILES: c1ccc(-c2nc3ccc4ccc(-n5c6ccccc6c6cc(-n7c8ccccc8c8ccccc87)ccc65)cc4c3nc2-c2ccccc2)cc1] | 72% |
| 397 | 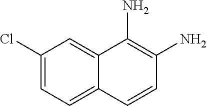 [SMILES: Nc1ccc2ccc(Cl)cc2c1N] | 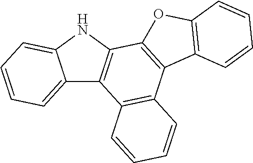 [SMILES: c1ccc2c(c1)[nH]c1c3oc4ccccc4c3c3ccccc3c21] | 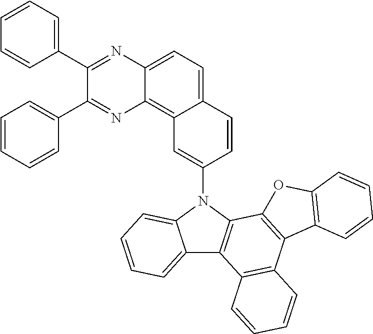 [SMILES: c1ccc(-c2nc3ccc4ccc(-n5c6ccccc6c6c7ccccc7c7c8ccccc8oc7c65)cc4c3nc2-c2ccccc2)cc1] | 81% |
| 403 | 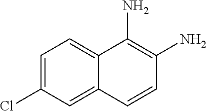 [SMILES: Nc1ccc2cc(Cl)ccc2c1N] | 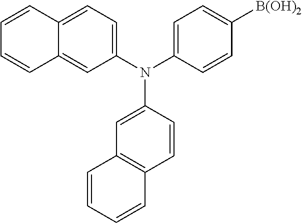 [SMILES: OB(O)c1ccc(N(c2ccc3ccccc3c2)c2ccc3ccccc3c2)cc1] | 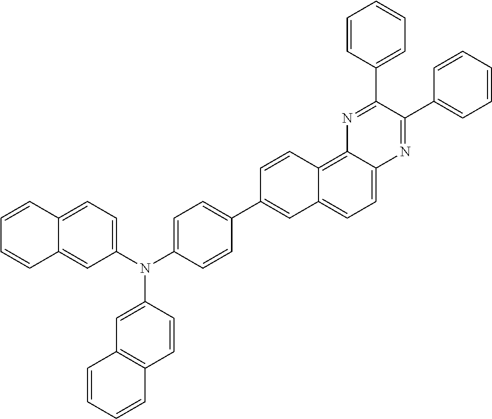 [SMILES: c1ccc(-c2nc3ccc4cc(-c5ccc(N(c6ccc7ccccc7c6)c6ccc7ccccc7c6)cc5)ccc4c3nc2-c2ccccc2)cc1] | 74% |
| 411 | 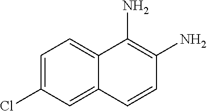 [SMILES: Nc1ccc2cc(Cl)ccc2c1N] | 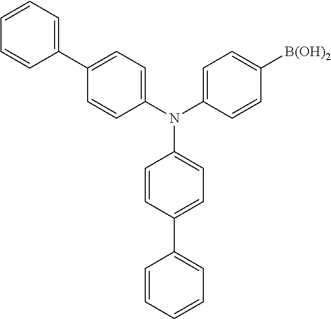 [SMILES: OB(O)c1ccc(N(c2ccc(-c3ccccc3)cc2)c2ccc(-c3ccccc3)cc2)cc1] | 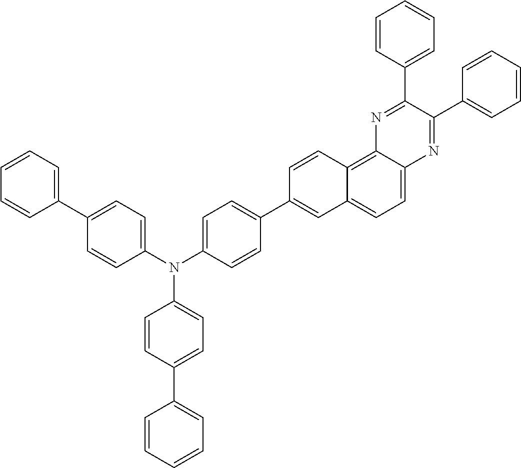 [SMILES: c1ccc(-c2ccc(N(c3ccc(-c4ccccc4)cc3)c3ccc(-c4ccc5c(ccc6nc(-c7ccccc7)c(-c7ccccc7)nc65)c4)cc3)cc2)cc1] | 73% |
| 424 | 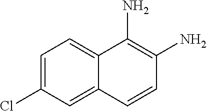 [SMILES: Nc1ccc2cc(Cl)ccc2c1N] | 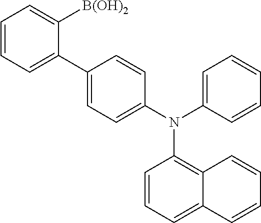 [SMILES: OB(O)c1ccccc1-c1ccc(N(c2ccccc2)c2cccc3ccccc23)cc1] | 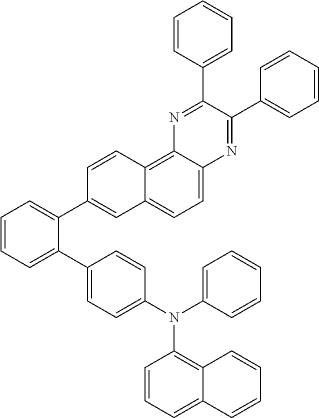 [SMILES: c1ccc(-c2nc3ccc4cc(-c5ccccc5-c5ccc(N(c6ccccc6)c6cccc7ccccc67)cc5)ccc4c3nc2-c2ccccc2)cc1] | 82% |
| 436 | 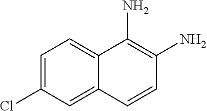 [SMILES: Nc1ccc2cc(Cl)ccc2c1N] | 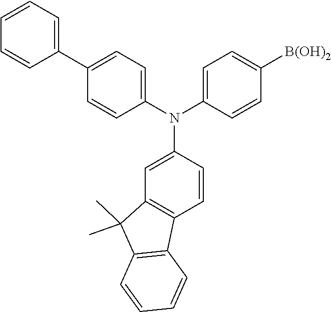 [SMILES: CC1(C)c2ccccc2-c2ccc(N(c3ccc(B(O)O)cc3)c3ccc(-c4ccccc4)cc3)cc21] | 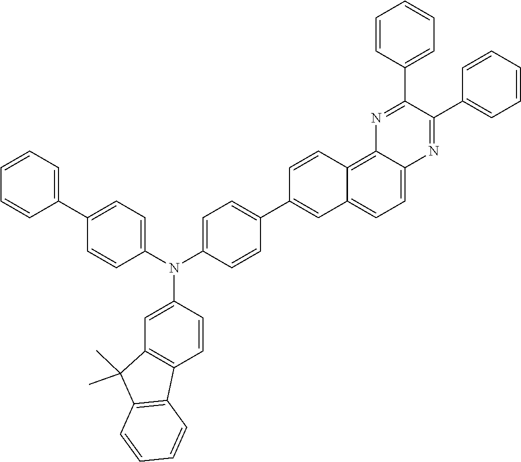 [SMILES: CC1(C)c2ccccc2-c2ccc(N(c3ccc(-c4ccccc4)cc3)c3ccc(-c4ccc5c(ccc6nc(-c7ccccc7)c(-c7ccccc7)nc65)c4)cc3)cc21] | 67% |
| 445 | 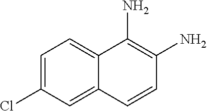 [SMILES: Nc1ccc2cc(Cl)ccc2c1N] | 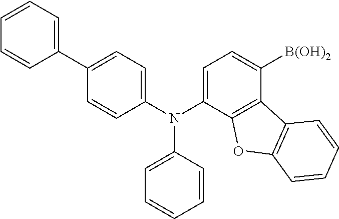 [SMILES: OB(O)c1ccc(N(c2ccccc2)c2ccc(-c3ccccc3)cc2)c2oc3ccccc3c12] | 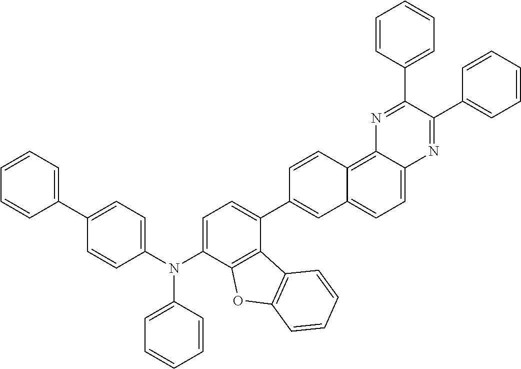 [SMILES: c1ccc(-c2ccc(N(c3ccccc3)c3ccc(-c4ccc5c(ccc6nc(-c7ccccc7)c(-c7ccccc7)nc65)c4)c4c3oc3ccccc34)cc2)cc1] | 77% |
| 449 | 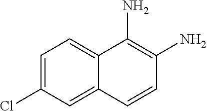 [SMILES: Nc1ccc2cc(Cl)ccc2c1N] | 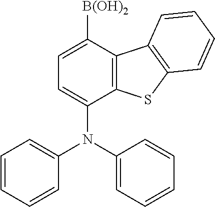 [SMILES: OB(O)c1ccc(N(c2ccccc2)c2ccccc2)c2sc3ccccc3c12] | 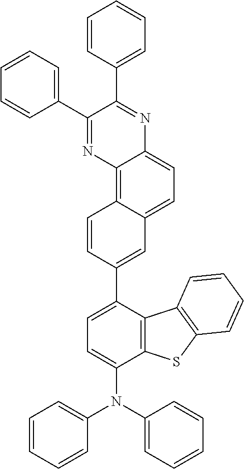 [SMILES: c1ccc(-c2nc3ccc4cc(-c5ccc(N(c6ccccc6)c6ccccc6)c6sc7ccccc7c56)ccc4c3nc2-c2ccccc2)cc1] | 64% |
| 459 | 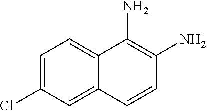 [SMILES: Nc1ccc2cc(Cl)ccc2c1N] | 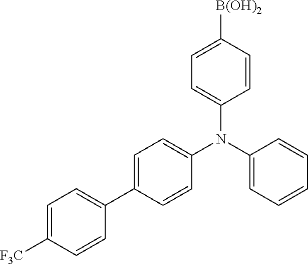 [SMILES: OB(O)c1ccc(N(c2ccccc2)c2ccc(-c3ccc(C(F)(F)F)cc3)cc2)cc1] | 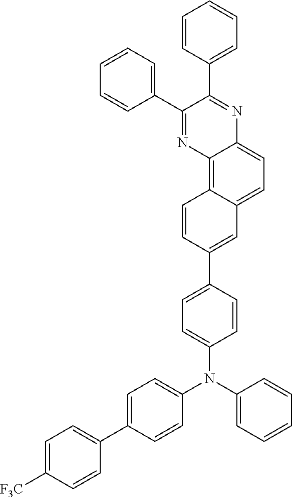 [SMILES: FC(F)(F)c1ccc(-c2ccc(N(c3ccccc3)c3ccc(-c4ccc5c(ccc6nc(-c7ccccc7)c(-c7ccccc7)nc65)c4)cc3)cc2)cc1] | 76% |
| 466 | 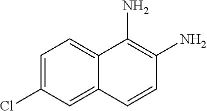 [SMILES: Nc1ccc2cc(Cl)ccc2c1N] | 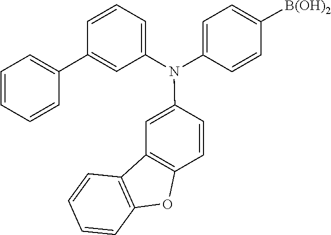 [SMILES: OB(O)c1ccc(N(c2cccc(-c3ccccc3)c2)c2ccc3oc4ccccc4c3c2)cc1] | 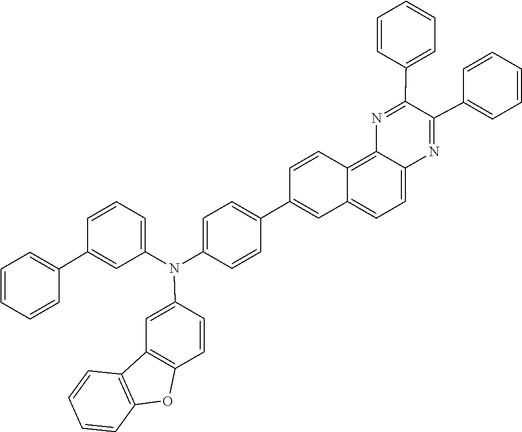 [SMILES: c1ccc(-c2cccc(N(c3ccc(-c4ccc5c(ccc6nc(-c7ccccc7)c(-c7ccccc7)nc65)c4)cc3)c3ccc4oc5ccccc5c4c3)c2)cc1] | 72% |
| 478 | 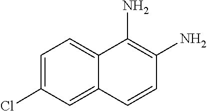 [SMILES: Nc1ccc2cc(Cl)ccc2c1N] | 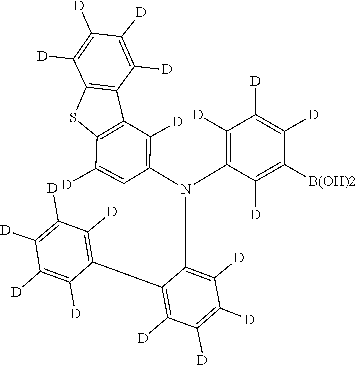 [SMILES: [2H]c1c([2H])c([2H])c(-c2c([2H])c([2H])c([2H])c([2H])c2N(c2cc([2H])c3sc4c([2H])c([2H])c([2H])c([2H])c4c3c2[2H])c2c([2H])c([2H])c([2H])c(B(O)O)c2[2H])c([2H])c1[2H]] | 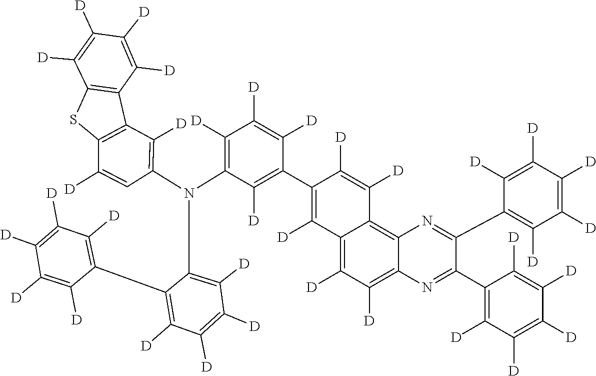 [SMILES: [2H]c1c([2H])c([2H])c(-c2nc3c([2H])c([2H])c4c([2H])c(-c5c([2H])c([2H])c([2H])c(N(c6cc([2H])c7sc8c([2H])c([2H])c([2H])c([2H])c8c7c6[2H])c6c([2H])c([2H])c([2H])c([2H])c6-c6c([2H])c([2H])c([2H])c([2H])c6[2H])c5[2H])c([2H])c([2H])c4c3nc2-c2c([2H])c([2H])c([2H])c([2H])c2[2H])c([2H])c1[2H]] | 77% |
| 493 | 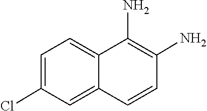 [SMILES: Nc1ccc2cc(Cl)ccc2c1N] | 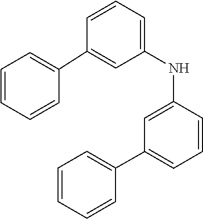 [SMILES: c1ccc(-c2cccc(Nc3cccc(-c4ccccc4)c3)c2)cc1] | 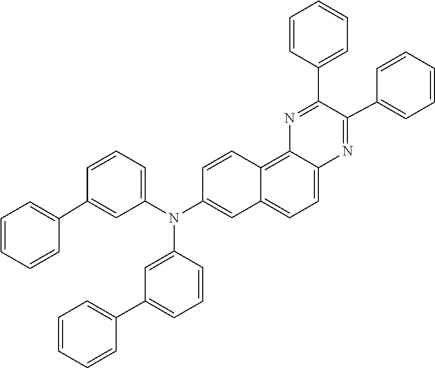 [SMILES: c1ccc(-c2cccc(N(c3cccc(-c4ccccc4)c3)c3ccc4c(ccc5nc(-c6ccccc6)c(-c6ccccc6)nc54)c3)c2)cc1] | 76% |
| 497 | 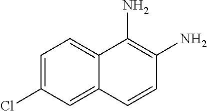 [SMILES: Nc1ccc2cc(Cl)ccc2c1N] | 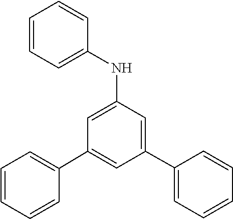 [SMILES: c1ccc(Nc2cc(-c3ccccc3)cc(-c3ccccc3)c2)cc1] | 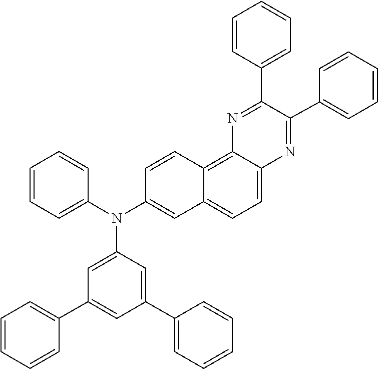 [SMILES: c1ccc(-c2cc(-c3ccccc3)cc(N(c3ccccc3)c3ccc4c(ccc5nc(-c6ccccc6)c(-c6ccccc6)nc54)c3)c2)cc1] | 84% |
| 513 | 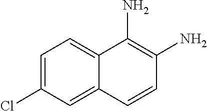 [SMILES: Nc1ccc2cc(Cl)ccc2c1N] | 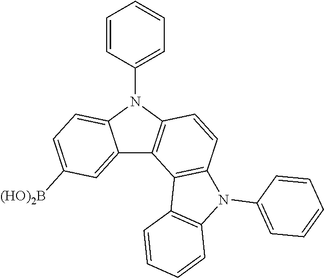 [SMILES: OB(O)c1ccc2c(c1)c1c3c4ccccc4n(-c4ccccc4)c3ccc1n2-c1ccccc1] | 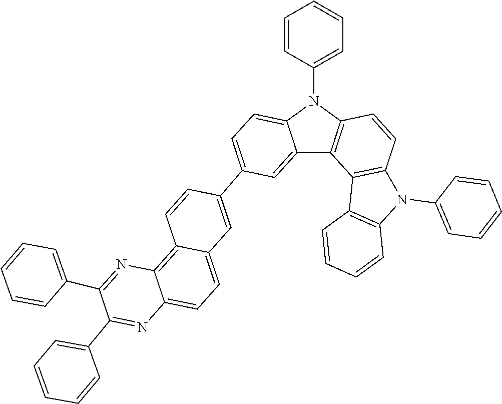 [SMILES: c1ccc(-c2nc3ccc4cc(-c5ccc6c(c5)c5c7c8ccccc8n(-c8ccccc8)c7ccc5n6-c5ccccc5)ccc4c3nc2-c2ccccc2)cc1] | 72% |
| 515 | 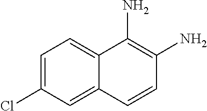 [SMILES: Nc1ccc2cc(Cl)ccc2c1N] | 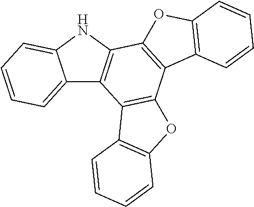 [SMILES: c1ccc2c(c1)[nH]c1c3oc4ccccc4c3c3oc4ccccc4c3c21] | 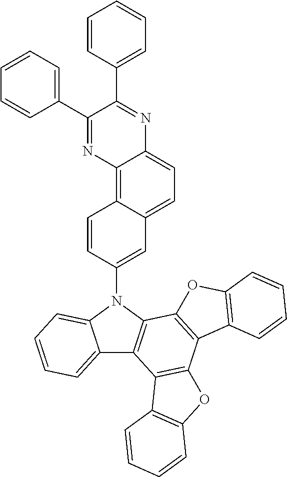 [SMILES: c1ccc(-c2nc3ccc4cc(-n5c6ccccc6c6c7c8ccccc8oc7c7c8ccccc8oc7c65)ccc4c3nc2-c2ccccc2)cc1] | 84% |
| 518 | 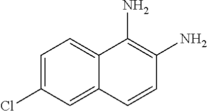 [SMILES: Nc1ccc2cc(Cl)ccc2c1N] | 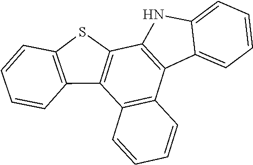 [SMILES: c1ccc2c(c1)[nH]c1c3sc4ccccc4c3c3ccccc3c21] | 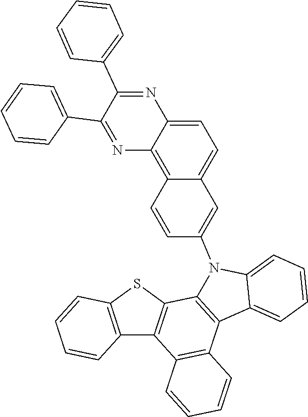 [SMILES: c1ccc(-c2nc3ccc4cc(-n5c6ccccc6c6c7ccccc7c7c8ccccc8sc7c65)ccc4c3nc2-c2ccccc2)cc1] | 76% |

### Preparation Example 2: Preparation of Target Compounds

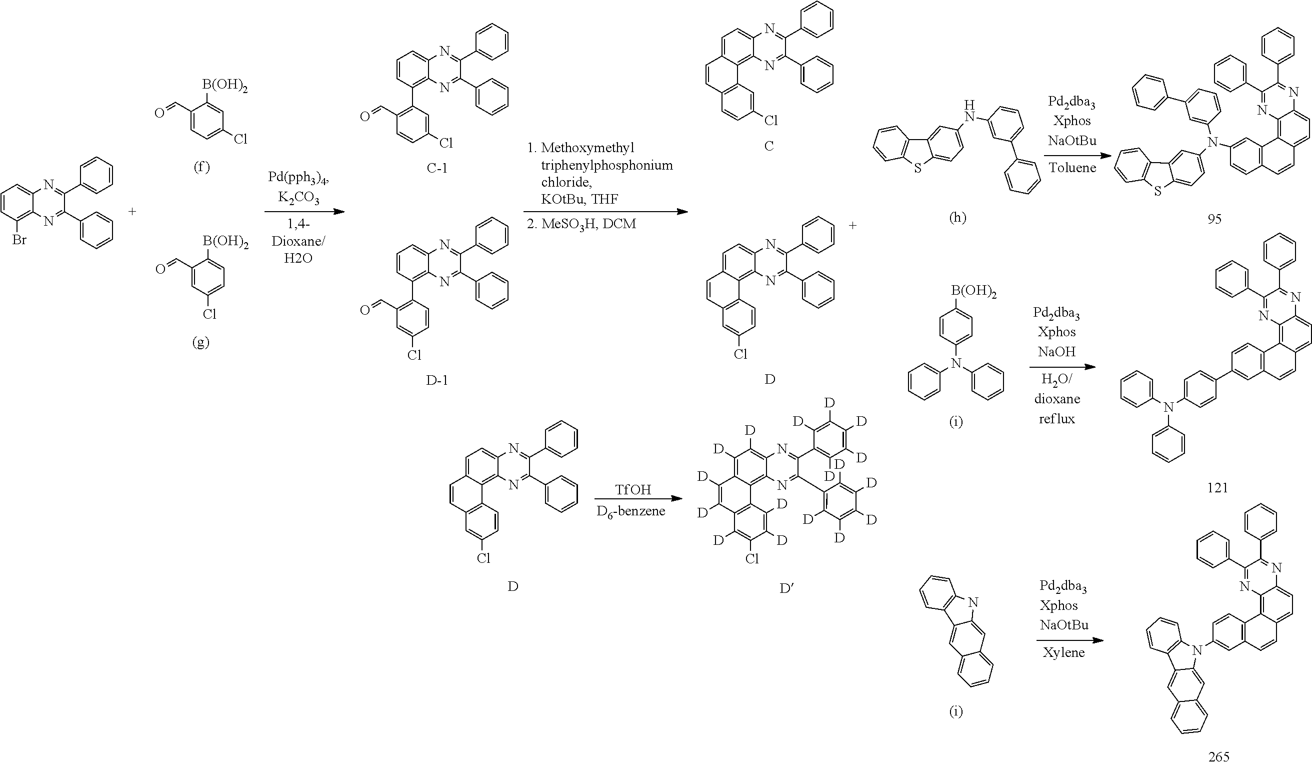

Using (5-chloro-2-formylphenyl) boronic acid that is Intermediate Compound (f) or (4-chloro-2-formylphenyl) boronic acid that is Intermediate Compound (g), 4-chloro-2-(2,3-diphenylquinoxalin-5-yl)benzaldehyde that is Compound C-1 or 5-chloro-2-(2,3-diphenylquinoxalin-5-yl)benzaldehyde that is Compound D-1 was prepared, respectively.

1) Preparation of Compound C-1

5-bromo-2,3-diphenylquinoxaline (10.0 g, 27.68 mmol), (5-chloro-2-formylphenyl) boronic acid 16.6 g, 35.98 mmol), tetrakis(triphenylphosphine) palladium (Pd(PPh₃)₄) (1.6 g, 1.38 mmol) and potassium carbonate (K₂CO₃) (7.65 g, 55.36 mmol) were introduced to 1,4-dioxane (100 ml) and water (H₂O) (20 ml) and dissolved therein, and then the mixture was refluxed for 1 hour and 30 minutes at 120° C. After the reaction was completed, the result was extracted by introducing distilled water and dichloromethane (DCM) thereto at room temperature. The organic layer was dried with magnesium sulfate (MgSO₄), and then the solvent was removed using a rotary evaporator. The reaction material was purified by silica, and then purified by column chromatography (DCM:Hex=1:5) to obtain target Compound C-1 (7 g, yield 92.10%).

2) Preparation of Compound C

(Methoxymethyl)tri-phenylphosphonium chloride (8.6 g, 24.94 mmol) was introduced to anhydrous tetrahydrofuran (THF) (35 ml) at −78° C. and dissolved therein, and after introducing potassium tert-butoxide (t-BuOK) (2.8 g, 24.94 mmol) thereto, the mixture was stirred for 30 minutes. Compound C-1 (7 g, 16.63 mmol) dissolved in anhydrous tetrahydrofuran (THF) (35 ml) was added dropwise thereto using a dropping funnel, and then the mixture was stirred for 3 hours at room temperature. After the reaction was completed, the result was extracted by introducing distilled water and dichloromethane (DCM) thereto at room temperature. The organic layer was dried with magnesium sulfate (MgSO₄), and then the solvent was removed using a rotary evaporator.

The reaction material was introduced to dichloromethane (DCM) (70 ml) and dissolved therein, and after introducing methanesulfonic acid (1.08 ml, 16.63 mmol) thereto, the mixture was refluxed for 2 hours at 60° C. After the reaction was completed, the result was extracted by introducing distilled water and dichloromethane (DCM) thereto at room temperature. The organic layer was dried with magnesium sulfate (MgSO₄), and then the solvent was removed using a rotary evaporator. The reaction material was purified by silica, and the reaction material was purified by column chromatography (DCM:Hex=1:4) to obtain target Compound C (5.2 g, yield 75.02%).
3) Preparation of Compound 95

Compound C (5.7 g, 13.62 mmol), N-([1,1′-biphenyl]-3-yl)dibenzo[b, d]thiophen-2-amine) (4.78 g, 13.62 mmol), tris(dibenzylideneacetone) dipalladium (Pd₂(dba)₃) (0.62 g, 0.68 mmol), Xphos (0.65 g, 1.36 mmol) and sodium tert-butoxide (t-BuONa) (2.62 g, 27.24 mmol) were introduced to toluene (57 ml) and dissolved therein, and then the mixture was refluxed for 6 hours at 100° C. After the reaction was completed, produced solid was filtered, washed with methanol (MeOH) and dried. The dried solid was dissolved in chloroform and purified by silica, and then the solvent was removed using a rotary evaporator. The result was recrystallized with acetone to obtain target Compound 95 (8.40 g, yield 84.25%).

4) Preparation of Compound 121

Compound D (5.7 g, 13.62 mmol), (4-(diphenylamino)phenyl) boronic acid (3.94 g, 13.62 mmol), tris(dibenzylideneacetone) dipalladium (Pd₂ (dba)₃) (0.62 g, 0.68 mmol), Xphos (0.65 g, 1.36 mmol) and sodium hydroxide (NaOH) (1.1 g, 27.24 mmol) were introduced to 1,4-dioxane (50 ml) and water (H₂O) (10 ml) and dissolved therein, and then the mixture was refluxed for 4 hours at 120° C. After the reaction was completed, produced solid was filtered, washed with methanol (MeOH) and dried. The dried solid was dissolved in chloroform and purified by silica, and then the solvent was removed using a rotary evaporator. The result was recrystallized with acetone to obtain target Compound 121 (7 g, yield 82.22%).

5) Preparation of Compound 265

Compound D (5.7 g, 13.62 mmol), 5-benzo[b]carbazole (2.9 g, 13.62 mmol), tris(dibenzylideneacetone) dipalladium (Pd₂ (dba)₃) (0.62 g, 0.68 mmol), Xphos (0.65 g, 1.36 mmol) and sodium tert-butoxide (t-BuONa) (2.62 g, 27.24 mmol) were introduced to xylene (50 ml) and dissolved therein, and then the mixture was refluxed for 5 hours at 150° ° C. After the reaction was completed, produced solid was filtered, washed with methanol (MeOH) and dried. The dried solid was dissolved in chloroform and purified by silica, and then the solvent was removed using a rotary evaporator. The result was recrystallized with acetone to obtain target Compound 265 (5.9 g, yield 72.48%).

6) Preparation of Compound D′ (substituted with deuterium in Intermediate D of Compound 199)

Compound D (5.0 g, 11.99 mmol) and benzend-d6 (50 mL) were introduced to a 500 mL round bottom flask, and trifluoromethanesulfonic acid (triflic acid, TfOH) (15.08 mL) was slowly added dropwise thereto. After that, the mixture was stirred for 1 hour at 100° C. After the reaction was completed, the result was cooled to room temperature, and washed with excess distilled water and methanol. After that, the result was dried, and then separated by a column under a condition of MC:Hex=1:2 to obtain Compound D′ (4.7 g, 11.9 mmol, yield 90.4%).

Target compounds were synthesized in the same manner as in Preparation Example 2, using Intermediates (f), (g), (h), (i) and (j) of the following Table 2 instead of Intermediate Compounds (f), (g), (h), (i) and (j) in Preparation Example 2.

TABLE 2

| Com- | | | | |
|---|---|---|---|---|
| pound | Intermediate | Intermediate | | |
| No. | (f) / (g) | (h) / (i) / (j) | Target Compound | Yield |
| 8 | 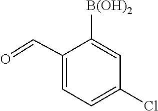 [SMILES: O=Cc1ccc(Cl)cc1B(O)O] | 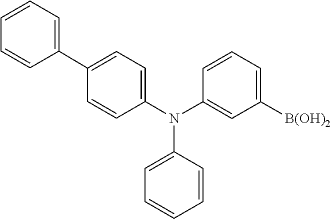 [SMILES: OB(O)c1cccc(N(c2ccccc2)c2ccc(-c3ccccc3)cc2)c1] | 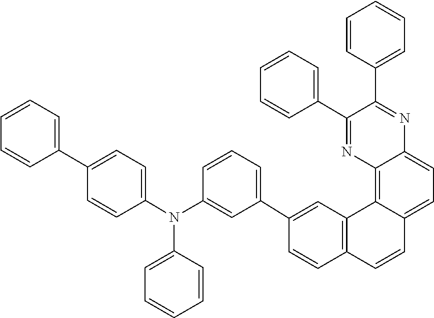 [SMILES: c1ccc(-c2ccc(N(c3ccccc3)c3cccc(-c4ccc5ccc6ccc7nc(-c8ccccc8)c(-c8ccccc8)nc7c6c5c4)c3)cc2)cc1] | 72% |
| 14 | 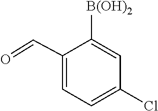 [SMILES: O=Cc1ccc(Cl)cc1B(O)O] | 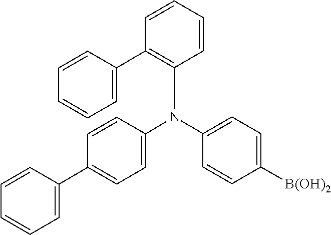 [SMILES: OB(O)c1ccc(N(c2ccc(-c3ccccc3)cc2)c2ccccc2-c2ccccc2)cc1] | 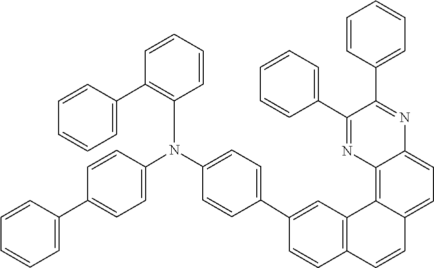 [SMILES: c1ccc(-c2ccc(N(c3ccc(-c4ccc5ccc6ccc7nc(-c8ccccc8)c(-c8ccccc8)nc7c6c5c4)cc3)c3ccccc3-c3ccccc3)cc2)cc1] | 76% |
| 37 | 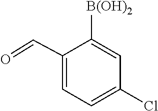 [SMILES: O=Cc1ccc(Cl)cc1B(O)O] | 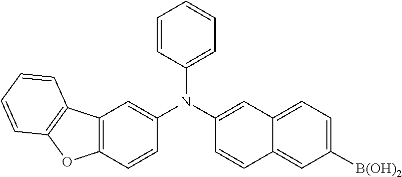 [SMILES: OB(O)c1ccc2cc(N(c3ccccc3)c3ccc4oc5ccccc5c4c3)ccc2c1] | 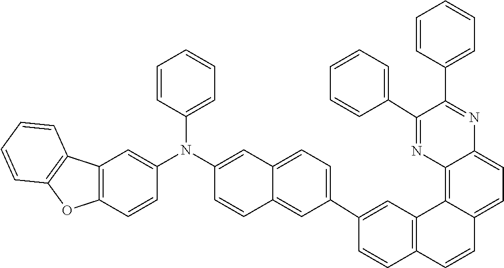 [SMILES: c1ccc(-c2nc3ccc4ccc5ccc(-c6ccc7cc(N(c8ccccc8)c8ccc9oc%10ccccc%10c9c8)ccc7c6)cc5c4c3nc2-c2ccccc2)cc1] | 84% |
| 50 | 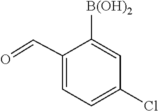 [SMILES: O=Cc1ccc(Cl)cc1B(O)O] | 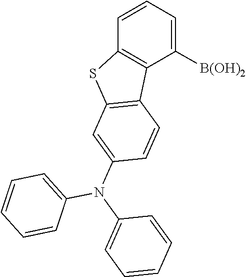 [SMILES: OB(O)c1cccc2sc3cc(N(c4ccccc4)c4ccccc4)ccc3c12] | 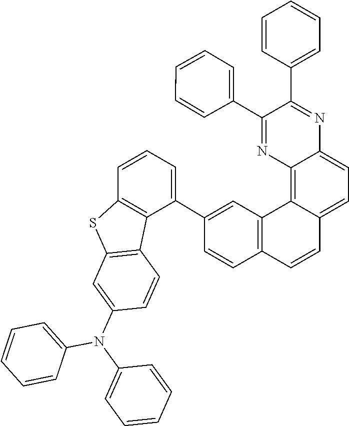 [SMILES: c1ccc(-c2nc3ccc4ccc5ccc(-c6cccc7sc8cc(N(c9ccccc9)c9ccccc9)ccc8c67)cc5c4c3nc2-c2ccccc2)cc1] | 81% |
| 58 | 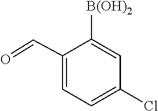 [SMILES: O=Cc1ccc(Cl)cc1B(O)O] | 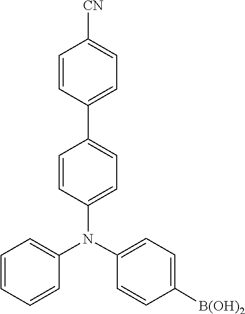 [SMILES: N#Cc1ccc(-c2ccc(N(c3ccccc3)c3ccc(B(O)O)cc3)cc2)cc1] | 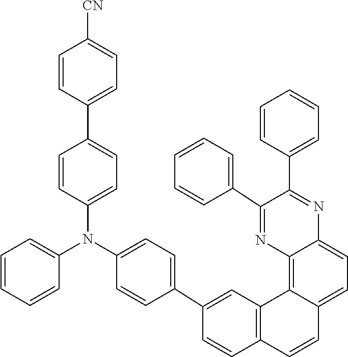 [SMILES: N#Cc1ccc(-c2ccc(N(c3ccccc3)c3ccc(-c4ccc5ccc6ccc7nc(-c8ccccc8)c(-c8ccccc8)nc7c6c5c4)cc3)cc2)cc1] | 71% |
| 60 | 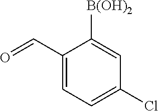 [SMILES: O=Cc1ccc(Cl)cc1B(O)O] | 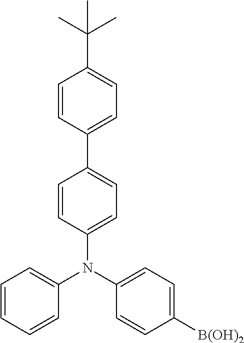 [SMILES: CC(C)(C)c1ccc(-c2ccc(N(c3ccccc3)c3ccc(B(O)O)cc3)cc2)cc1] | 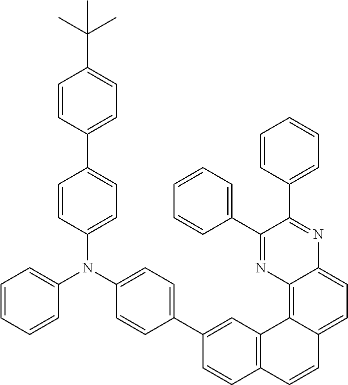 [SMILES: CC(C)(C)c1ccc(-c2ccc(N(c3ccccc3)c3ccc(-c4ccc5ccc6ccc7nc(-c8ccccc8)c(-c8ccccc8)nc7c6c5c4)cc3)cc2)cc1] | 84% |
| 70 | 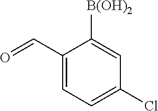 [SMILES: O=Cc1ccc(Cl)cc1B(O)O] | 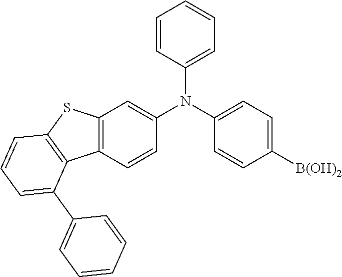 [SMILES: OB(O)c1ccc(N(c2ccccc2)c2ccc3c(c2)sc2cccc(-c4ccccc4)c23)cc1] | 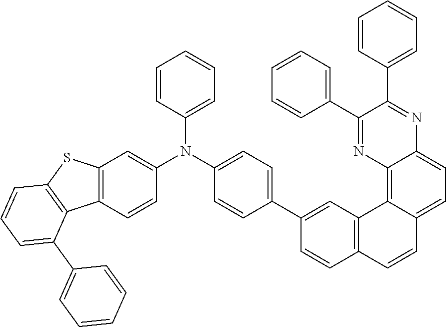 [SMILES: c1ccc(-c2nc3ccc4ccc5ccc(-c6ccc(N(c7ccccc7)c7ccc8c(c7)sc7cccc(-c9ccccc9)c78)cc6)cc5c4c3nc2-c2ccccc2)cc1] | 86% |
| 77 | 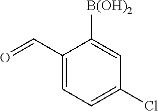 [SMILES: O=Cc1ccc(Cl)cc1B(O)O] | 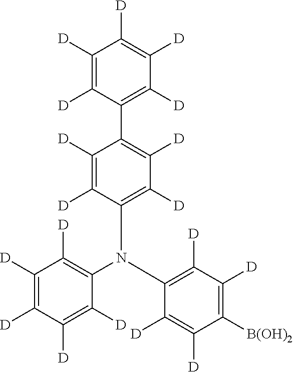 [SMILES: [2H]c1c([2H])c([2H])c(-c2c([2H])c([2H])c(N(c3c([2H])c([2H])c([2H])c([2H])c3[2H])c3c([2H])c([2H])c(B(O)O)c([2H])c3[2H])c([2H])c2[2H])c([2H])c1[2H]] | 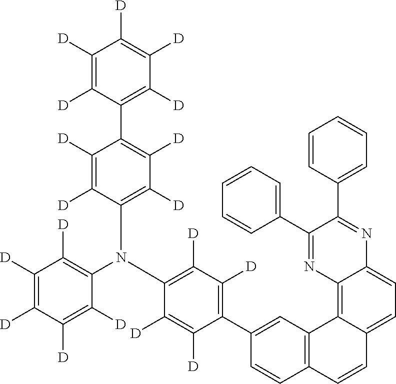 [SMILES: [2H]c1c([2H])c([2H])c(-c2c([2H])c([2H])c(N(c3c([2H])c([2H])c([2H])c([2H])c3[2H])c3c([2H])c([2H])c(-c4ccc5ccc6ccc7nc(-c8ccccc8)c(-c8ccccc8)nc7c6c5c4)c([2H])c3[2H])c([2H])c2[2H])c([2H])c1[2H]] | 66% |
| 95 | 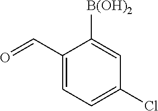 [SMILES: O=Cc1ccc(Cl)cc1B(O)O] | 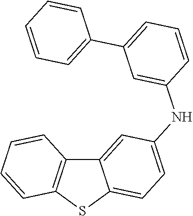 [SMILES: c1ccc(-c2cccc(Nc3ccc4sc5ccccc5c4c3)c2)cc1] | 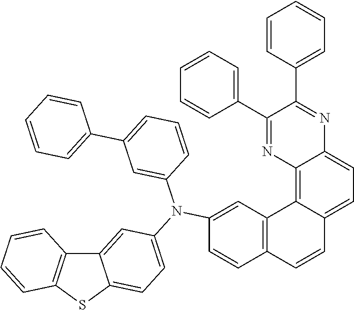 [SMILES: c1ccc(-c2cccc(N(c3ccc4sc5ccccc5c4c3)c3ccc4ccc5ccc6nc(-c7ccccc7)c(-c7ccccc7)nc6c5c4c3)c2)cc1] | 81% |
| 101 | 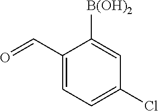 [SMILES: O=Cc1ccc(Cl)cc1B(O)O] | 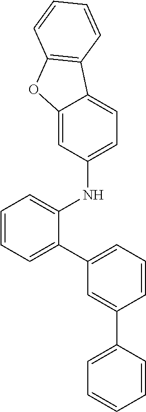 [SMILES: c1ccc(-c2cccc(-c3ccccc3Nc3ccc4c(c3)oc3ccccc34)c2)cc1] | 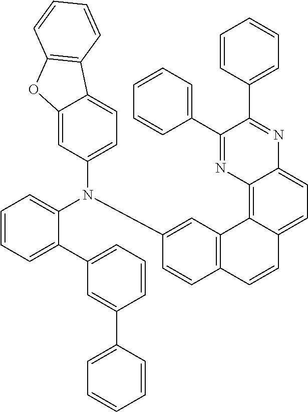 [SMILES: c1ccc(-c2cccc(-c3ccccc3N(c3ccc4c(c3)oc3ccccc34)c3ccc4ccc5ccc6nc(-c7ccccc7)c(-c7ccccc7)nc6c5c4c3)c2)cc1] | 74% |
| 120 | 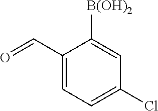 [SMILES: O=Cc1ccc(Cl)cc1B(O)O] | 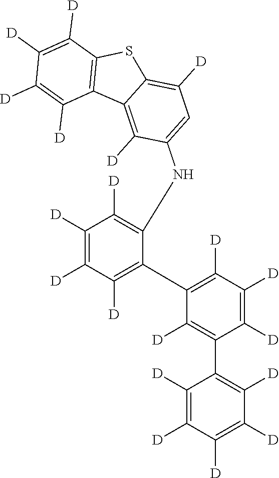 [SMILES: [2H]c1c([2H])c([2H])c(-c2c([2H])c([2H])c([2H])c(-c3c([2H])c([2H])c([2H])c([2H])c3Nc3cc([2H])c4sc5c([2H])c([2H])c([2H])c([2H])c5c4c3[2H])c2[2H])c([2H])c1[2H]] | 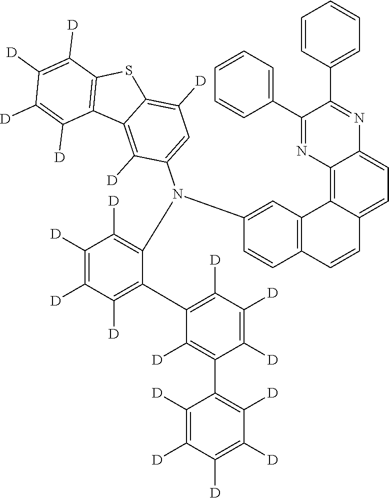 [SMILES: [2H]c1c([2H])c([2H])c(-c2c([2H])c([2H])c([2H])c(-c3c([2H])c([2H])c([2H])c([2H])c3N(c3ccc4ccc5ccc6nc(-c7ccccc7)c(-c7ccccc7)nc6c5c4c3)c3cc([2H])c4sc5c([2H])c([2H])c([2H])c([2H])c5c4c3[2H])c2[2H])c([2H])c1[2H]] | 73% |
| 121 | 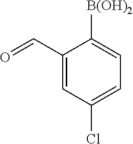 [SMILES: O=Cc1cc(Cl)ccc1B(O)O] | 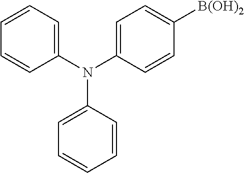 [SMILES: OB(O)c1ccc(N(c2ccccc2)c2ccccc2)cc1] | 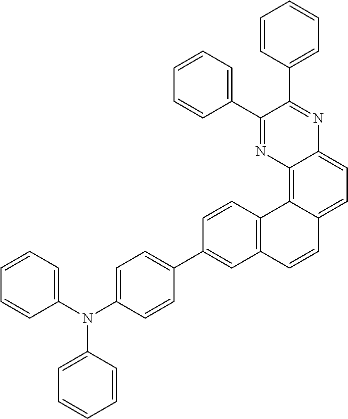 [SMILES: c1ccc(-c2nc3ccc4ccc5cc(-c6ccc(N(c7ccccc7)c7ccccc7)cc6)ccc5c4c3nc2-c2ccccc2)cc1] | 82% |
| 140 | 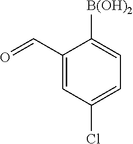 [SMILES: O=Cc1cc(Cl)ccc1B(O)O] | 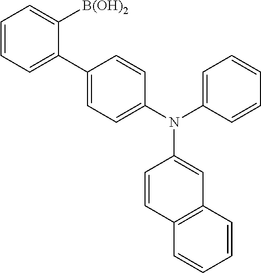 [SMILES: OB(O)c1ccccc1-c1ccc(N(c2ccccc2)c2ccc3ccccc3c2)cc1] | 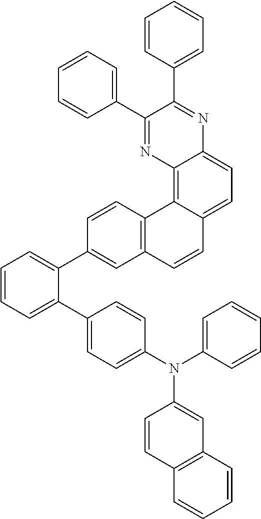 [SMILES: c1ccc(-c2nc3ccc4ccc5cc(-c6ccccc6-c6ccc(N(c7ccccc7)c7ccc8ccccc8c7)cc6)ccc5c4c3nc2-c2ccccc2)cc1] | 67% |
| 147 | 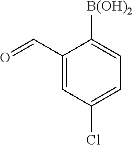 [SMILES: O=Cc1cc(Cl)ccc1B(O)O] | 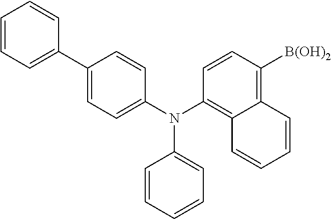 [SMILES: OB(O)c1ccc(N(c2ccccc2)c2ccc(-c3ccccc3)cc2)c2ccccc12] | 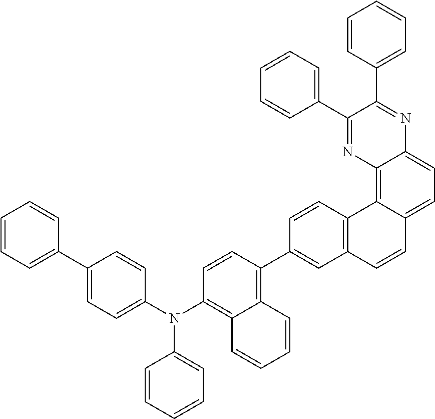 [SMILES: c1ccc(-c2ccc(N(c3ccccc3)c3ccc(-c4ccc5c(ccc6ccc7nc(-c8ccccc8)c(-c8ccccc8)nc7c65)c4)c4ccccc34)cc2)cc1] | 72% |
| 150 | 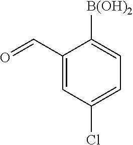 [SMILES: O=Cc1cc(Cl)ccc1B(O)O] | 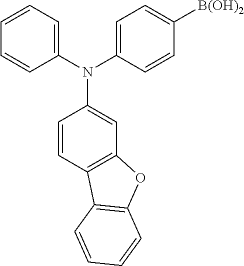 [SMILES: OB(O)c1ccc(N(c2ccccc2)c2ccc3c(c2)oc2ccccc23)cc1] | 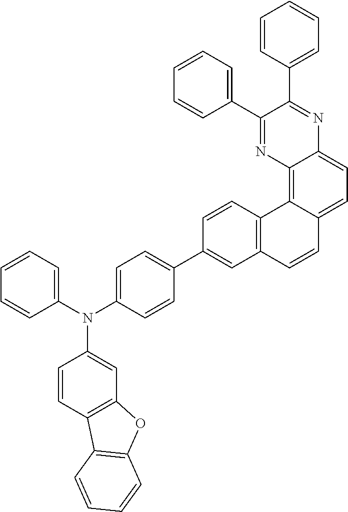 [SMILES: c1ccc(-c2nc3ccc4ccc5cc(-c6ccc(N(c7ccccc7)c7ccc8c(c7)oc7ccccc78)cc6)ccc5c4c3nc2-c2ccccc2)cc1] | 70% |
| 155 | 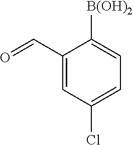 [SMILES: O=Cc1cc(Cl)ccc1B(O)O] | 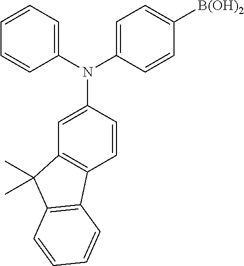 [SMILES: CC1(C)c2ccccc2-c2ccc(N(c3ccccc3)c3ccc(B(O)O)cc3)cc21] | 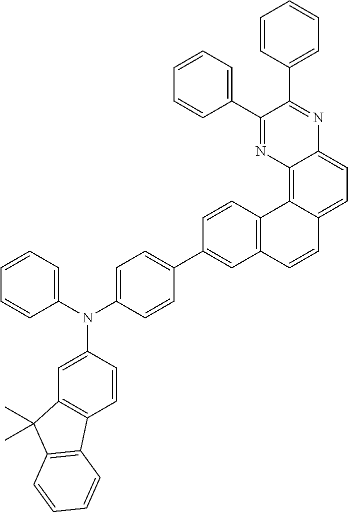 [SMILES: CC1(C)c2ccccc2-c2ccc(N(c3ccccc3)c3ccc(-c4ccc5c(ccc6ccc7nc(-c8ccccc8)c(-c8ccccc8)nc7c65)c4)cc3)cc21] | 76% |
| 167 | 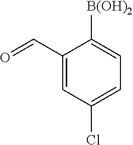 [SMILES: O=Cc1cc(Cl)ccc1B(O)O] | 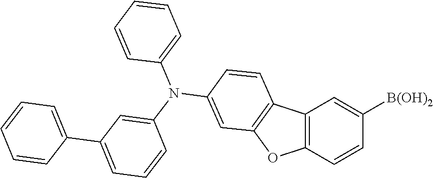 [SMILES: OB(O)c1ccc2oc3cc(N(c4ccccc4)c4cccc(-c5ccccc5)c4)ccc3c2c1] | 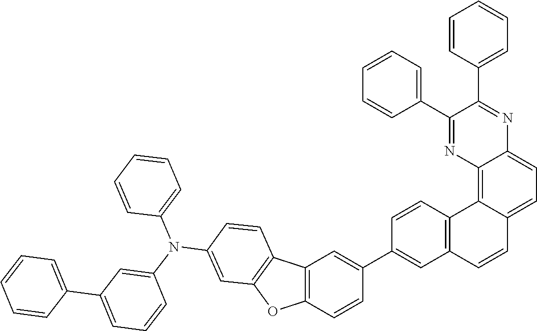 [SMILES: c1ccc(-c2cccc(N(c3ccccc3)c3ccc4c(c3)oc3ccc(-c5ccc6c(ccc7ccc8nc(-c9ccccc9)c(-c9ccccc9)nc8c76)c5)cc34)c2)cc1] | 68% |
| 174 | 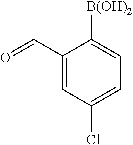 [SMILES: O=Cc1cc(Cl)ccc1B(O)O] | 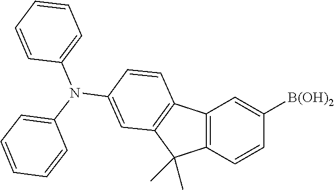 [SMILES: CC1(C)c2ccc(B(O)O)cc2-c2ccc(N(c3ccccc3)c3ccccc3)cc21] | 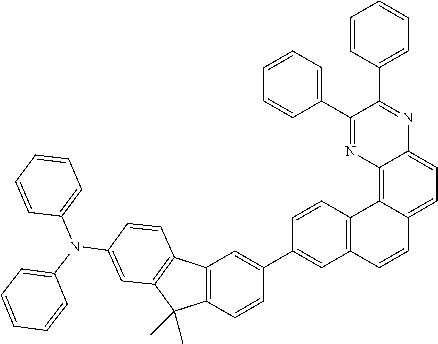 [SMILES: CC1(C)c2ccc(-c3ccc4c(ccc5ccc6nc(-c7ccccc7)c(-c7ccccc7)nc6c54)c3)cc2-c2ccc(N(c3ccccc3)c3ccccc3)cc21] | 82% |
| 177 | 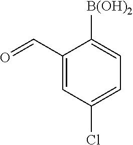 [SMILES: O=Cc1cc(Cl)ccc1B(O)O] | 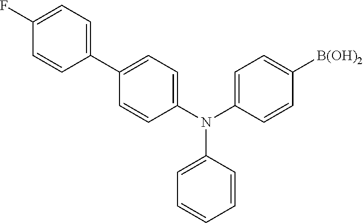 [SMILES: OB(O)c1ccc(N(c2ccccc2)c2ccc(-c3ccc(F)cc3)cc2)cc1] | 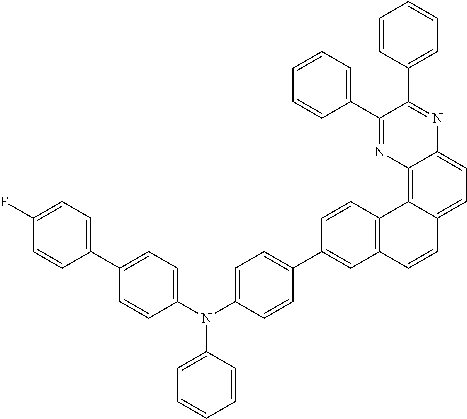 [SMILES: Fc1ccc(-c2ccc(N(c3ccccc3)c3ccc(-c4ccc5c(ccc6ccc7nc(-c8ccccc8)c(-c8ccccc8)nc7c65)c4)cc3)cc2)cc1] | 75% |
| 179 | 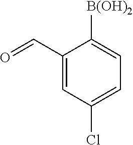 [SMILES: O=Cc1cc(Cl)ccc1B(O)O] | 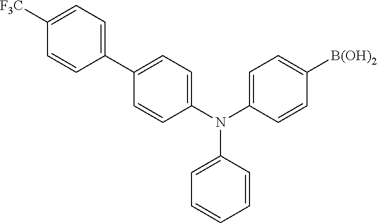 [SMILES: OB(O)c1ccc(N(c2ccccc2)c2ccc(-c3ccc(C(F)(F)F)cc3)cc2)cc1] | 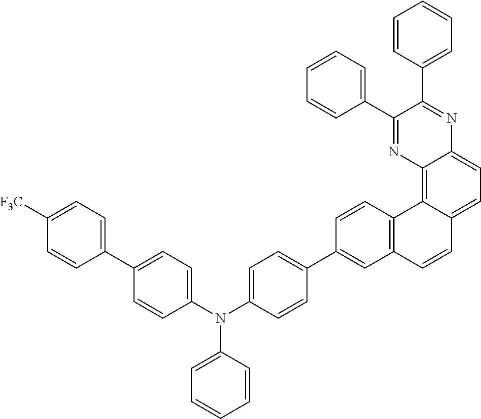 [SMILES: FC(F)(F)c1ccc(-c2ccc(N(c3ccccc3)c3ccc(-c4ccc5c(ccc6ccc7nc(-c8ccccc8)c(-c8ccccc8)nc7c65)c4)cc3)cc2)cc1] | 66% |
| 187 | 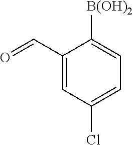 [SMILES: O=Cc1cc(Cl)ccc1B(O)O] | 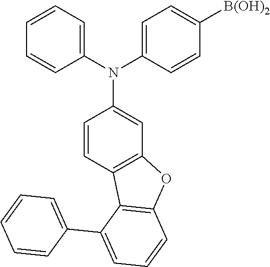 [SMILES: OB(O)c1ccc(N(c2ccccc2)c2ccc3c(c2)oc2cccc(-c4ccccc4)c23)cc1] | 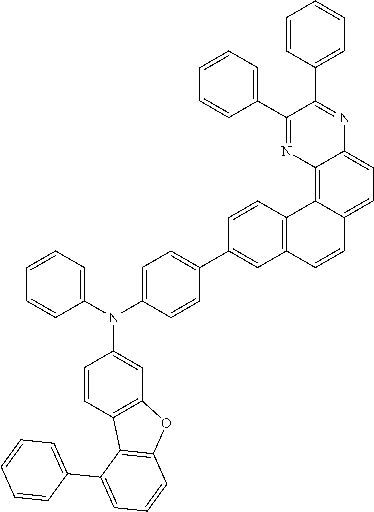 [SMILES: c1ccc(-c2nc3ccc4ccc5cc(-c6ccc(N(c7ccccc7)c7ccc8c(c7)oc7cccc(-c9ccccc9)c78)cc6)ccc5c4c3nc2-c2ccccc2)cc1] | 79% |
| 199 | 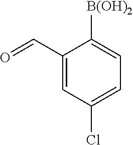 [SMILES: O=Cc1cc(Cl)ccc1B(O)O] | 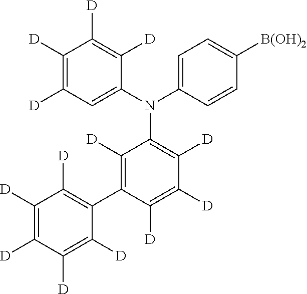 [SMILES: [2H]c1cc(N(c2ccc(B(O)O)cc2)c2c([2H])c([2H])c([2H])c(-c3c([2H])c([2H])c([2H])c([2H])c3[2H])c2[2H])c([2H])c([2H])c1[2H]] | 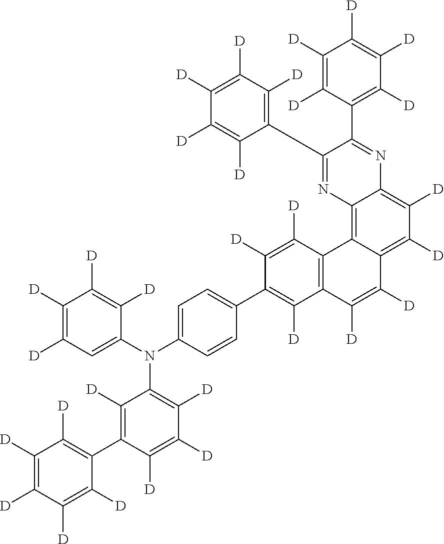 [SMILES: [2H]c1cc(N(c2ccc(-c3c([2H])c([2H])c4c(c3[2H])c([2H])c([2H])c3c([2H])c([2H])c5nc(-c6c([2H])c([2H])c([2H])c([2H])c6[2H])c(-c6c([2H])c([2H])c([2H])c([2H])c6[2H])nc5c34)cc2)c2c([2H])c([2H])c([2H])c(-c3c([2H])c([2H])c([2H])c([2H])c3[2H])c2[2H])c([2H])c([2H])c1[2H]] | 86% |
| 227 | 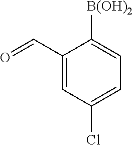 [SMILES: O=Cc1cc(Cl)ccc1B(O)O] | 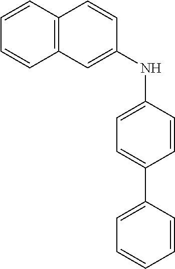 [SMILES: c1ccc(-c2ccc(Nc3ccc4ccccc4c3)cc2)cc1] | 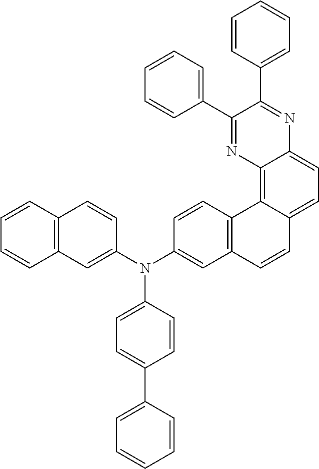 [SMILES: c1ccc(-c2ccc(N(c3ccc4ccccc4c3)c3ccc4c(ccc5ccc6nc(-c7ccccc7)c(-c7ccccc7)nc6c54)c3)cc2)cc1] | 66% |
| 242 | 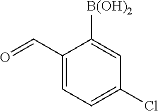 [SMILES: O=Cc1ccc(Cl)cc1B(O)O] | 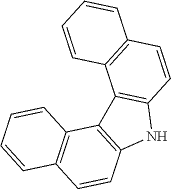 [SMILES: c1ccc2c(c1)ccc1[nH]c3ccc4ccccc4c3c12] | 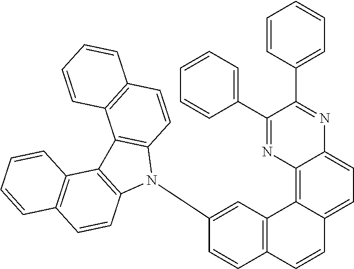 [SMILES: c1ccc(-c2nc3ccc4ccc5ccc(-n6c7ccc8ccccc8c7c7c8ccccc8ccc76)cc5c4c3nc2-c2ccccc2)cc1] | 82% |
| 254 | 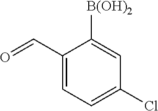 [SMILES: O=Cc1ccc(Cl)cc1B(O)O] | 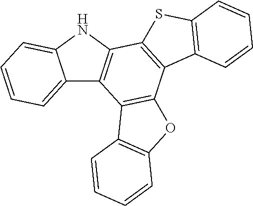 [SMILES: c1ccc2c(c1)[nH]c1c3sc4ccccc4c3c3oc4ccccc4c3c21] | 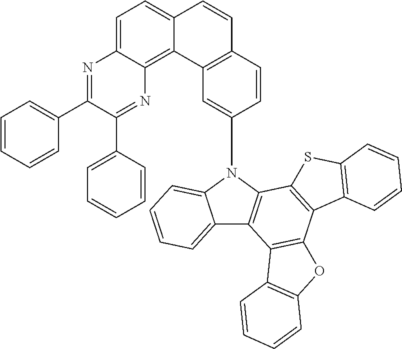 [SMILES: c1ccc(-c2nc3ccc4ccc5ccc(-n6c7ccccc7c7c8c9ccccc9oc8c8c9ccccc9sc8c76)cc5c4c3nc2-c2ccccc2)cc1] | 69% |
| 256 | 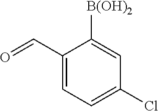 [SMILES: O=Cc1ccc(Cl)cc1B(O)O] | 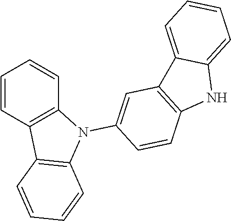 [SMILES: c1ccc2c(c1)[nH]c1ccc(-n3c4ccccc4c4ccccc43)cc12] | 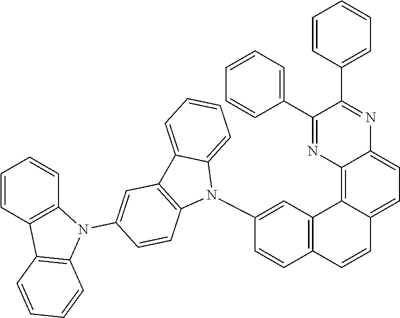 [SMILES: c1ccc(-c2nc3ccc4ccc5ccc(-n6c7ccccc7c7cc(-n8c9ccccc9c9ccccc98)ccc76)cc5c4c3nc2-c2ccccc2)cc1] | 75% |
| 265 | 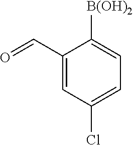 [SMILES: O=Cc1cc(Cl)ccc1B(O)O] | 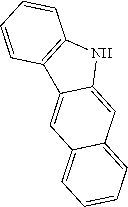 [SMILES: c1ccc2cc3c(cc2c1)[nH]c1ccccc13] | 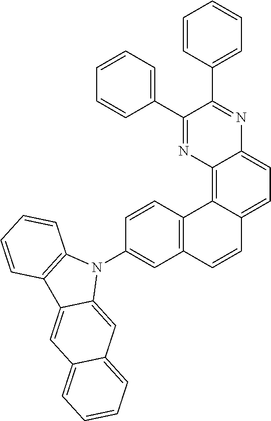 [SMILES: c1ccc(-c2nc3ccc4ccc5cc(-n6c7ccccc7c7cc8ccccc8cc76)ccc5c4c3nc2-c2ccccc2)cc1] | 70% |
| 270 | 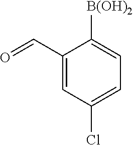 [SMILES: O=Cc1cc(Cl)ccc1B(O)O] | 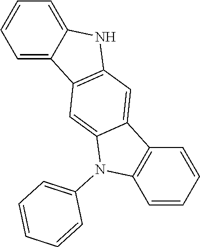 [SMILES: c1ccc(-n2c3ccccc3c3cc4[nH]c5ccccc5c4cc32)cc1] | 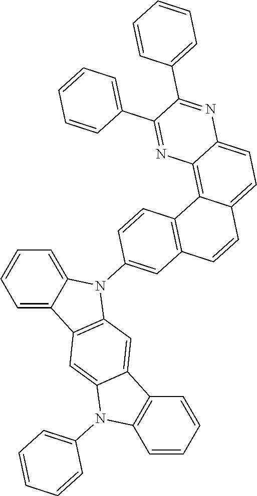 [SMILES: c1ccc(-c2nc3ccc4ccc5cc(-n6c7ccccc7c7cc8c(cc76)c6ccccc6n8-c6ccccc6)ccc5c4c3nc2-c2ccccc2)cc1] | 73% |
| 271 | 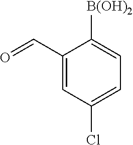 [SMILES: O=Cc1cc(Cl)ccc1B(O)O] | 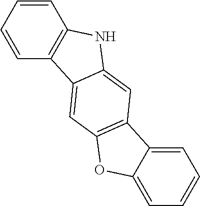 [SMILES: c1ccc2c(c1)[nH]c1cc3c(cc12)oc1ccccc13] | 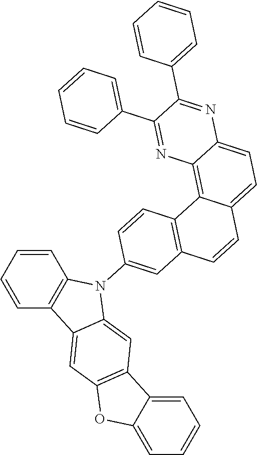 [SMILES: c1ccc(-c2nc3ccc4ccc5cc(-n6c7ccccc7c7cc8oc9ccccc9c8cc76)ccc5c4c3nc2-c2ccccc2)cc1] | 68 |
| 272 | 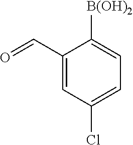 [SMILES: O=Cc1cc(Cl)ccc1B(O)O] | 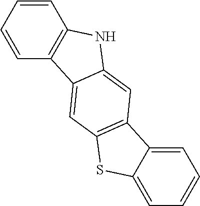 [SMILES: c1ccc2c(c1)[nH]c1cc3c(cc12)sc1ccccc13] | 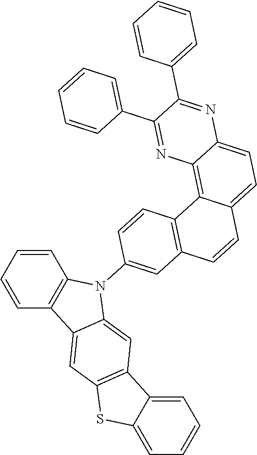 [SMILES: c1ccc(-c2nc3ccc4ccc5cc(-n6c7ccccc7c7cc8sc9ccccc9c8cc76)ccc5c4c3nc2-c2ccccc2)cc1] | 76% |

### Preparation Example 3: Preparation of Target Compounds

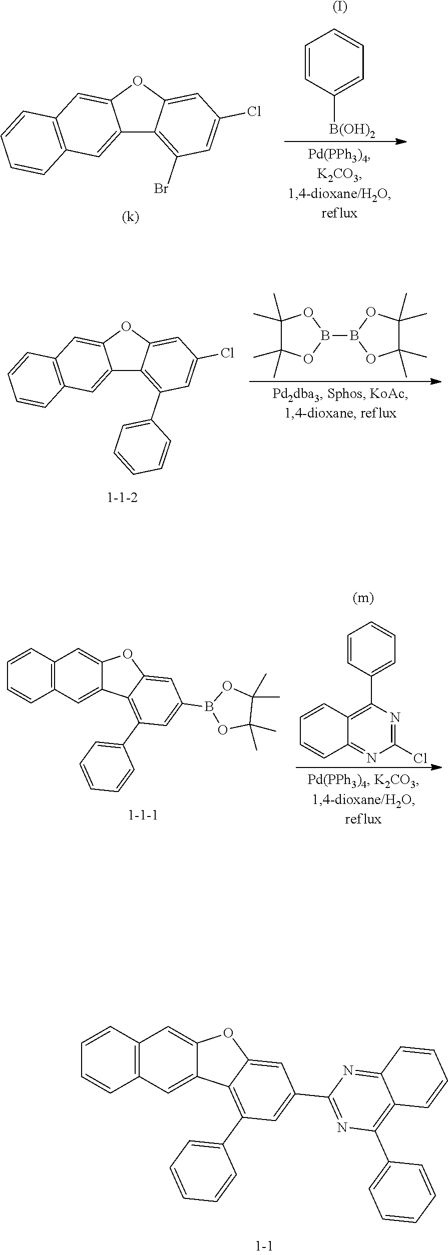

1) Preparation of Compound 1-1-2

1-bromo-3-chloro-benzo[b]naphtho[2,3-d]furan (6.0 g, 18.09 mmol), phenylboronic acid (1) (2.65 g, 21.71 mmol), tetrakis(triphenylphosphine) palladium (Pd(PPh₃)₄) (1.23 g, 1.07 mmol) and potassium carbonate (K₂CO₃) (5.89 g, 42.62 mmol) were introduced to 1,4-dioxane (30 mL) and water (H₂O) (6 mL) and dissolved therein, and then the mixture was refluxed for 24 hours. After the reaction was completed, the result was extracted by introducing distilled water and dichloromethane (DCM) thereto at room temperature. The organic layer was dried with magnesium sulfate (MgSO₄), and then the solvent was removed using a rotary evaporator. The solvent-removed reaction material was purified by silica, and then purified by column chromatography (DCM:Hex=1:5) to obtain Compound 1-1-2 (5 g, yield 45%). 2) Preparation of Compound 1-1-1

Compound 1-1-2 (5.0 g, 15.21 mmol), bis(pinacolato)diboron (4.3 g, 16.73 mmol, tris(dibenzylideneacetone) dipalladium (0) (Pd₂ (dba)₃) (0.44 g, 1.07 mmol), dicyclohexyl-(2′,6′-dimethoxybiphenyl-2-yl)phosphine (Sphos) (0.4 g, 0.958 mmol) and potassium acetate (KOAc) (1.9 g, 19.16 mmol) were introduced to 1,4-dioxane (50 mL) and dissolved therein, and then the mixture was refluxed for 5 hours. After the reaction was completed, the result was extracted by introducing distilled water and dichloromethane (DCM) thereto at room temperature. The organic layer was dried with magnesium sulfate (MgSO₄), and then the solvent was removed using a rotary evaporator. The solvent-removed reaction material was purified by silica, and then recrystallized with methanol to obtain Compound 1-1-1 (3.27 g, yield 56%).

3) Preparation of Target Compound 1-1

Compound 1-1-1 (3.27 g, 7.8 mmol), 2-chloro-4-phenylquinazoline (m) (1.9 g, 7.8 mmol), tetrakis(triphenylphosphine) palladium (Pd(PPh₃)₄) (0.31 g, 0.27 mmol) and potassium carbonate (K₂CO₃) (1.47 g, 10.66 mmol) were introduced to 1,4-dioxane (25 mL) and water (H₂O) (5 mL) and dissolved therein, and then the mixture was refluxed for 3 hours. After the reaction was completed, produced solid was filtered, washed with distilled water and dried. The dried solid was dissolved in chloroform and purified by silica, and then the solvent was removed using a rotary evaporator. The result was recrystallized with acetone to obtain target Compound 1-1 (3.1 g, yield 80%).

Target compounds of the following Table 3 were synthesized in the same manner as in Preparation Example 3, using Intermediates (k), (l) and (m) of the following Table 3 instead of 1-bromo-3-chloro-benzo[b]naphtho[2,3-d]furan, phenylboronic acid and 2-chloro-4-phenylquinazoline, respectively, in Preparation Example 3.

TABLE 3

| Com- | | | | | |
|---|---|---|---|---|---|
| pound | Intermediate | Intermediate | Intermediate | | |
| No. | (k) | (l) | (m) | Target Compound | Yield |
| 1-1 | 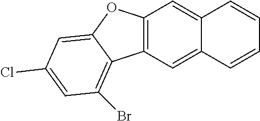 [SMILES: Clc1cc(Br)c2c(c1)oc1cc3ccccc3cc12] | 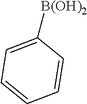 | 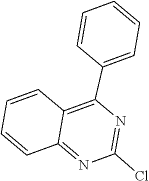 [SMILES: Clc1nc(-c2ccccc2)c2ccccc2n1] | 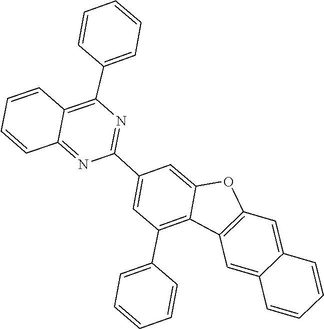 [SMILES: c1ccc(-c2nc(-c3cc(-c4ccccc4)c4c(c3)oc3cc5ccccc5cc34)nc3ccccc23)cc1] | 88% |
| 1-6 | 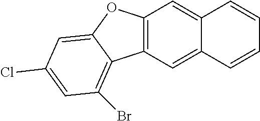 [SMILES: Clc1cc(Br)c2c(c1)oc1cc3ccccc3cc12] | 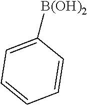 | 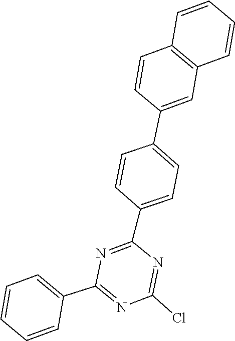 [SMILES: Clc1nc(-c2ccccc2)nc(-c2ccc(-c3ccc4ccccc4c3)cc2)n1] | 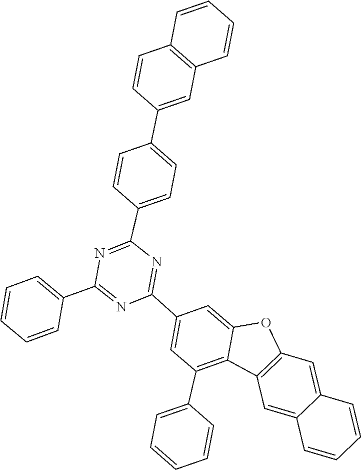 [SMILES: c1ccc(-c2nc(-c3ccc(-c4ccc5ccccc5c4)cc3)nc(-c3cc(-c4ccccc4)c4c(c3)oc3cc5ccccc5cc34)n2)cc1] | 84 |
| 1-10 | 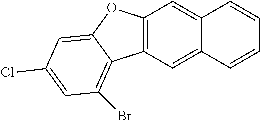 [SMILES: Clc1cc(Br)c2c(c1)oc1cc3ccccc3cc12] | 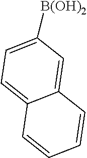 [SMILES: OB(O)c1ccc2ccccc2c1] | 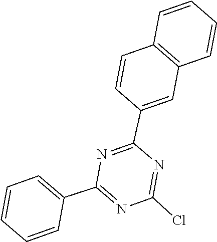 [SMILES: Clc1nc(-c2ccccc2)nc(-c2ccc3ccccc3c2)n1] | 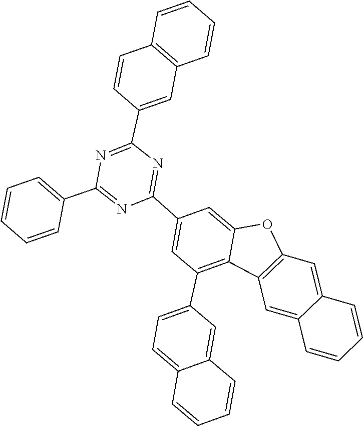 [SMILES: c1ccc(-c2nc(-c3ccc4ccccc4c3)nc(-c3cc(-c4ccc5ccccc5c4)c4c(c3)oc3cc5ccccc5cc34)n2)cc1] | 79% |
| 1-22 | 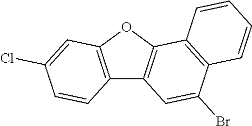 [SMILES: Clc1ccc2c(c1)oc1c3ccccc3c(Br)cc21] | 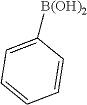 | 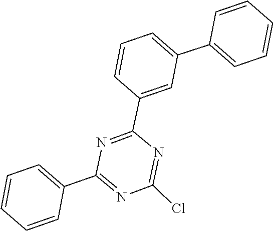 [SMILES: Clc1nc(-c2ccccc2)nc(-c2cccc(-c3ccccc3)c2)n1] | 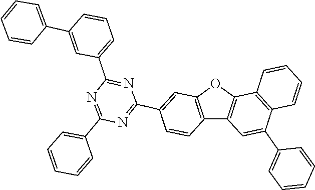 [SMILES: c1ccc(-c2cccc(-c3nc(-c4ccccc4)nc(-c4ccc5c(c4)oc4c6ccccc6c(-c6ccccc6)cc54)n3)c2)cc1] | 86% |
| 1-32 | 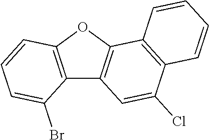 [SMILES: Clc1cc2c(oc3cccc(Br)c32)c2ccccc12] | 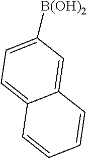 [SMILES: OB(O)c1ccc2ccccc2c1] | 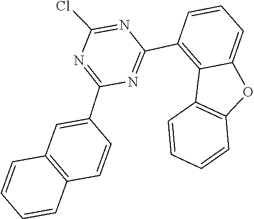 [SMILES: Clc1nc(-c2ccc3ccccc3c2)nc(-c2cccc3oc4ccccc4c23)n1] | 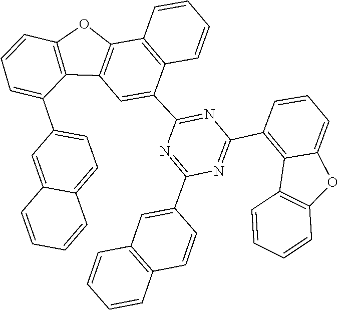 [SMILES: c1ccc2cc(-c3nc(-c4cc5c(oc6cccc(-c7ccc8ccccc8c7)c65)c5ccccc45)nc(-c4cccc5oc6ccccc6c45)n3)ccc2c1] | 80% |
| 1-41 | 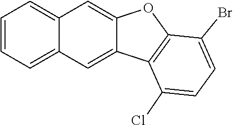 [SMILES: Clc1ccc(Br)c2oc3cc4ccccc4cc3c12] | 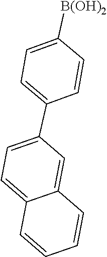 [SMILES: OB(O)c1ccc(-c2ccc3ccccc3c2)cc1] | 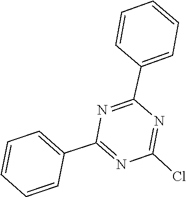 [SMILES: Clc1nc(-c2ccccc2)nc(-c2ccccc2)n1] | 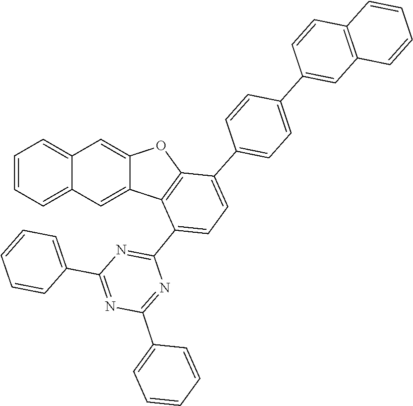 [SMILES: c1ccc(-c2nc(-c3ccccc3)nc(-c3ccc(-c4ccc(-c5ccc6ccccc6c5)cc4)c4oc5cc6ccccc6cc5c34)n2)cc1] | 83% |
| 1-44 | 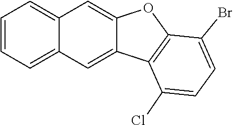 [SMILES: Clc1ccc(Br)c2oc3cc4ccccc4cc3c12] | 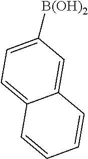 [SMILES: OB(O)c1ccc2ccccc2c1] | 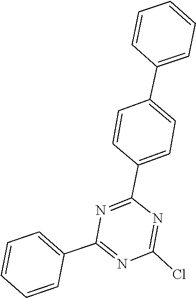 [SMILES: Clc1nc(-c2ccccc2)nc(-c2ccc(-c3ccccc3)cc2)n1] | 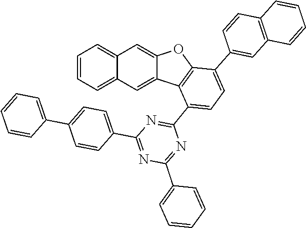 [SMILES: c1ccc(-c2ccc(-c3nc(-c4ccccc4)nc(-c4ccc(-c5ccc6ccccc6c5)c5oc6cc7ccccc7cc6c45)n3)cc2)cc1] | 75% |
| 1-50 | 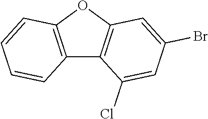 [SMILES: Clc1cc(Br)cc2oc3ccccc3c12] | 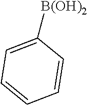 | 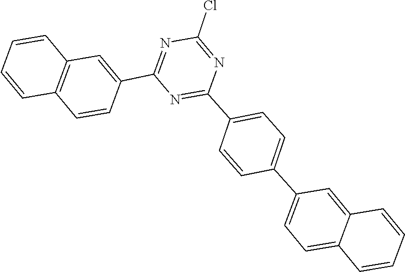 [SMILES: Clc1nc(-c2ccc(-c3ccc4ccccc4c3)cc2)nc(-c2ccc3ccccc3c2)n1] | 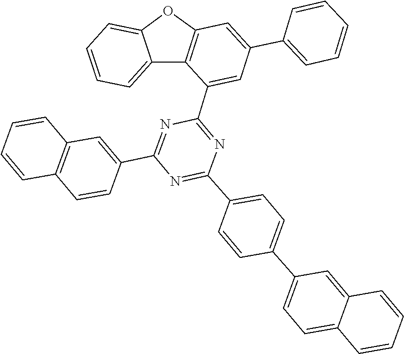 [SMILES: c1ccc(-c2cc(-c3nc(-c4ccc(-c5ccc6ccccc6c5)cc4)nc(-c4ccc5ccccc5c4)n3)c3c(c2)oc2ccccc23)cc1] | 69% |
| 1-52 | 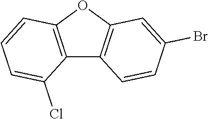 [SMILES: Clc1cccc2oc3cc(Br)ccc3c12] | 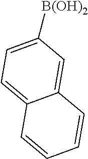 [SMILES: OB(O)c1ccc2ccccc2c1] | 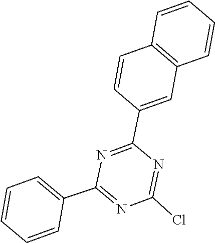 [SMILES: Clc1nc(-c2ccccc2)nc(-c2ccc3ccccc3c2)n1] | 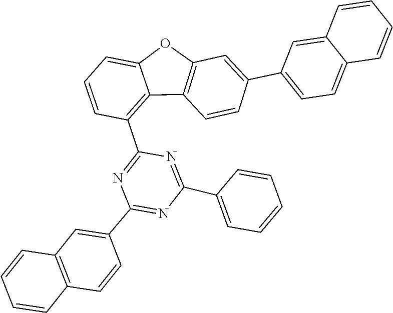 [SMILES: c1ccc(-c2nc(-c3ccc4ccccc4c3)nc(-c3cccc4oc5cc(-c6ccc7ccccc7c6)ccc5c34)n2)cc1] | 77% |
| 1-54 | 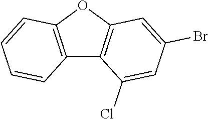 [SMILES: Clc1cc(Br)cc2oc3ccccc3c12] | 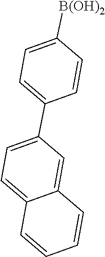 [SMILES: OB(O)c1ccc(-c2ccc3ccccc3c2)cc1] | 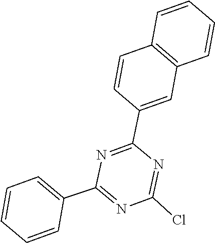 [SMILES: Clc1nc(-c2ccccc2)nc(-c2ccc3ccccc3c2)n1] | 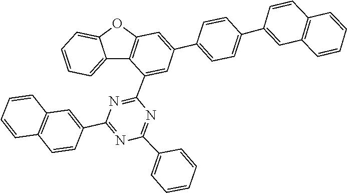 [SMILES: c1ccc(-c2nc(-c3ccc4ccccc4c3)nc(-c3cc(-c4ccc(-c5ccc6ccccc6c5)cc4)cc4oc5ccccc5c34)n2)cc1] | 79% |

The rest of compounds other than the compounds described in Preparation Examples 1 to 3 and Tables 1 to 3 were also prepared in the same manner as in the preparation examples described above, and the synthesis results are shown in the following Table 4 and Table 5. The following Table 4 shows measurement values of 1H NMR (CDCl₃, 300 Mz) of the compounds, and the following Table 5 shows measurement values of FD-mass spectrometry (FD-MS: field desorption mass spectrometry).

TABLE 4

| NO | ¹H NMR (CDCl₃, 300 Mz) |
|---|---|
| 8 | 9.27 (s, 1H), 8.37 (d, 1H), 8.35 (d, 1H), 8.11 (d, 1H), 8.03 |
| | (d, 4H), 7.92 (d, 1H), 7.82 (d, 1H), 7.75 (d, 3H), 7.59-7.24 |
| | (m, 19H), 7.08 (d, 2H), 7.00 (t, 1H) |
| 14 | 9.27 (s, 1H), 8.37 (d, 1H), 8.35 (d, 1H), 8.11 (d, 1H), 8.10 |
| | (d, 1H), 8.03 (d, 4H), 7.92 (d, 1H), 7.82 (d, 1H), 7.75 |
| | (d, 3H), 7. 59-7.27 (m, 22H), 7.14 (t, 1H), 7.08 (d, 1H) |
| 37 | 9.27 (s, 1H), 8.37 (d, 1H), 8.35 (d, 1H), 8.22 (s, 1H), 8.11 |
| | (d, 1H), 8.03 (d, 4H), 7.92 (d, 1H), 7.98 (d, 1H), 7.84 |
| | (d, 1H), 7.82 (d, 1H), 7.75 (d, 1H), 7.59 (t, 2H), 7.56 (d, 1H), |
| | 7.54 (d, 1H), 7.56-7.00 (m, 17H) |
| 50 | 9.27 (s, 1H), 8.37 (d, 1H), 8.35 (d, 1H), 8.11 (d, 1H), 8.03 |
| | (d, 4H), 7.96 (d, 1H), 7.92 (d, 1H), 7.82 (d, 1H), 7.75 (d, 1H), |
| | 7.74 (d, 1H), 7.67 (d, 1H), 7.64 (s, 1H), 7.59 (t, 2H), 7.50 |
| | (t, 1H), 7.43 (d, 1H), 7.32 (t, 4H), 7.24 (t, 4H), 7.08 (d, 4H), |
| | 7.00 (t, 2H) |
| 58 | 9.27 (s, 1H). 8.37 (d, 1H), 8.35 (d, 1H), 8.11 (d, 1H), 8.03 |
| | (d, 4H), 7.93 (d, 2H), 7.92 (d, 1H), 7.84 (d, 2H), 7.82 (d, 1H), |
| | 7.75 (d, 1H), 7.59 (t, 2H), 7.55 (d, 4H), 7.32 (t, 4H), 7.27 |
| | (d, 4H), 7.24 (t, 2H), 7.08 (d, 2H), 7.00 (t, 1H) |
| 60 | 9.27 (s, 1H). 8.37 (d, 1H), 8.35 (d, 1H), 8.11 (d, 1H), 8.03 |
| | (d, 4H), 7.92 (d, 1H), 7.82 (d, 1H), 7.75 (d, 1H), 7.59 (t, 2H), |
| | 7.55 (d, 4H), 7.38-7.24 (m, 14H), 7.08 (d, 2H), 7.00 (t, 1H) |
| 70 | 9.27 (s, 1H), 8.37 (d, 1H), 8.35 (d, 1H), 8.11 (d, 1H), 8.03 |
| | (d, 4H), 7.96 (d, 1H), 7.92 (d, 1H), 7.82-7.24 (m, 24H), 7.08 |
| | (d, 2H), 7.00 (t, 1H) |
| 95 | 8.71 (s, 1H), 8.45 (d, 1H), 8.11 (d, 1H), 8.07 (d, 1H), 8.03 |
| | (d, 4H), 7.95 (s, 1H), 7.92 (d, 1H), 7.86 (d, 2H), 7.75 (d, 3H), |
| | (7.55 (d, 1H), 7.59-7.27 m, 15H), 7.18 (d, 1H), 7.17 (d, 1H) |
| 101 | 8.71 (s, 1H), 8.11 (d, 1H), 8.10 (d, 1H), 8.07 (d, 1H), 8.03 |
| | (d, 5H), 7.98 (d, 1H), 7.94 (s, 1H), 7.92 (d, 1H), 7.82 (d, 1H), |
| | 7.75 (d, 3H), 7.71 (t, 1H), 7.61-7.27 (m, 18H), 7.14 (t, 1H), |
| | 6.94 (d, 1H) |
| 121 | 9.11 (d, 1H), 8.46 (s, 1H), 8.43 (d, 1H), 8.11 (d, 1H), 8.03 |
| | (d, 4H), 7.82 (d, 1H), 7.59 (t, 2H), 7.55 (d, 2H), 7.32 (t, 4H), |
| | 7.27 (d, 2H), 7.24 (t, 4H), 7.08 (d, 4H), 7.00 (t, 2H) |
| 140 | 9.11 (d, 1H), 8.46 (s, 1H), 8.43 (d, 1H), 8.25 (d, 2H), 8.11 |
| | (d, 1H), 8.03 (d, 4H), 7.82 (d, 1H), 7.71-7.24 (m, 20H), 7,11 |
| | (s, 1H), 7.08 (d, 2H), 7.00 (t, 1H) |
| 147 | 9.11 (d, 1H), 8.95 (d, 1H), 8.46 (s, 1H), 8.43 (d, 1H), 8.27 |
| | (d, 1H), 8.11 (d, 1H), 8.03 (d, 4H), 7.92 (d, 2H), 7.82 (d, 1H), |
| | 7.75 (d, 2H), 7.59-7.24 (m, 19H), 7.08 (d, 2H), 7.00 (t, 1H) |
| 150 | 9.11 (d, 1H), 8.46 (s, 1H), 8.43 (d, 1H), 8.11 (d, 1H), 8.04 |
| | (s, 1H), 8.03 (d, 4H), 7.98 (d, 1H), 7.92 (d, 2H), 7.82 (d, 1H), |
| | 7.59 (t, 2H), 7.55 (d, 3H), 7.54 (d, 1H), 7.39-7.24 (m, 10H), |
| | 7.08 (d, 2H), 7.00 (t, 1H), 6.91 (d, 1H) |
| 155 | 9.11 (d, 1H), 8.46 (s, 1H), 8.43 (d, 1H), 8.11 (d, 1H), 8.03 |
| | (d, 4H), 7.92 (d, 2H), 7.90 (d, 1H), 7.86 (d, 1H), 7.82 (d, 1H), |
| | 7.59 (t, 2H), 7.55 (d, 3H), 7.38-7.24 (m, 12H), 7.08 (d, 2H), |
| | 7.00 (t, 1H) |
| 167 | 9.11 (d, 1H), 8.46 (d, 1H), 8.43 (d, 1H), 8.11 (d, 1H), 8.04 |
| | (s, 1H), 8.03 (d, 4H), 7.83-7.17 (m, 22H), 7.08 (d, 2H), 7.00 |
| | (t, 1H), 6.91 (d, 1H) |
| 174 | 9.11 (d, 1H), 8.46 (d, 1H), 8.43 (d, 1H), 8.18 (s, 1H), 8.11 |
| | (d, 1H), 8.03 (d, 4H), 7.92 (d, 1H), 7.86 (d, 1H), 7.82 (d, 1H), |
| | 7.74 (d, 1H), 7.68 (d, 1H), 7.59 (t, 2H), 7.33 (s, 1H), 7.32 |
| | (t, 4H), 7.24-7.00 (m, 11H) |
| 177 | 9.11 (d, 1H), 8.46 (d, 1H), 8.43 (d, 1H), 8.11 (d, 1H), 8.03 |
| | (d, 4H), 7.92 (d, 2H), 7.82 (d, 1H), 7.59 (t, 2H), 7.55 (d, 4H), |
| | 7.52 (d, 2H), 7.32 (t, 4H), 7.27 (d, 4H), 7.24 (t, 2H), 7.13 |
| | (d, 2H), 7.08 (d, 2H), 7.00 (t, 1H) |
| 187 | 9.11 (d, 1H), 8.46 (d, 1H), 8.43 (d, 1H), 8.11 (d, 1H), 8.04 |
| | (s, 1H), 8.03 (d, 4H), 7.92 (d, 2H), 7.82 (d, 1H), 7.79 (d, 2H), |
| | 7.59-7.24 (m, 19H), 7.08 (d, 2H), 7.00 (t, 1H), 6.91 (d, 1H) |
| 203 | 8.88 (d, 1H), 8.11 (d, 1H), 8.03 (d, 4H), 7.92 (d, 2H), 7.90 |
| | (s, 2H), 7.86 (d, 1H), 7.82 (d, 1H), 7.66 (d, 1H), 7.59 (t, 2H), |
| | 7.55 (d, 1H), 7.38 (t, 1H), 7.33 (s, 1H), 7.32 (t, 4H), 7.28 |
| | (t, 2H), 7.24 (t, 2H), 7.16 (d, 1H), 7.08 (d, 2H), 7.00 (t, 1H) |
| 227 | 8.88 (d, 1H), 8.11 (d, 1H), 8.03 (d, 4H), 7.92 (d, 2H), 7.90 |
| | (s, 1H), 7.82 (d, 1H), 7.75-7.32 (m, 21H), 7.27 (d,2 H), 7.11 |
| | (s, 1H) |
| 237 | 8.88 (d, 1H), 8.11 (d, 1H), 8.03 (d, 4H), 7.92 (d, 2H), 7.90 |
| | (s, 1H), 7.82 (d, 1H), 7.75-7.24 (m, 22H), 7.08 (d, 2H), 7.00 |
| | (t, 1H) |
| 242 | 9.05 (s, 1H), 8.54 (d, 2H), 8.25 (d, 1H), 8.11 (D, 1H), 8.03 |
| | (d, 4H), 7.99 (d, 2H), 7.94 (d, 1H), 7.92 (d, 1H), 7.82 (d, 1H), |
| | 7.75 (d, 1H), 7.61 (t, 1H), 7.59 (t, 2H), 7.53 (t, 2H), 7.52 |
| | (d, 2H), 7.32 (t, 4H), 7.31 (d, 1H), 7.16 (d, 1H) |
| 254 | 9.05 (s, 1H), 8.55 (d, 1H), 8.45 (d, 1H), 8.25 (d, 1H), |
| | 8.11-7.75 (m, 11H), 7.59 (t, 2H), 7.56 (t, 1H), 7.54 (d, 1H), |
| | 7.52 (d, 1H), 7.39-7.31 (m, 7H), 7.16 (t, 1H), 7.11 (t, 1H) |
| 256 | 9.05 (s, 1H), 8.55 (d, 2H), 8.25 (d, 1H), 8.19 (d, 1H), 8.11 |
| | (d, 1H), 8.03 (d, 4H), 7.82 (d. 1H), 7.75 (d, 1H), 7.67 |
| | (s, 1H), 7.59 (t, 2H), 7.54 (d, 1H), 7.52 (d, 1H), 7.40 (d, 1H), |
| | 7.32 (d, 4H), 7.21 (d, 1H), 7.20 (t, 1H), 7.18 (t, 1H), 7.16 |
| | (t, 2H), 7.11 (t, 2H) |
| 265 | 9.06 (d, 1H), 8.28 (d, 1H), 8.24 (s, 1H), 8.19 (d, 1H), 8.11 |
| | (d, 1H), 8.03 (d, 4H), 8.00 (d, 1H), 7.92 (d, 2H), 7.84 (d, 1H), |
| | 7.82 (d, 1H), 7.75 (t, 1H), 7.59 (t, 2H), 7.55 (d, 1H), 7.48 |
| | (t, 1H), 7.40 (s, 2H), 7.32 (t, 4H), 7.20 (t, 1H), 7.18 (t, 1H) |
| 270 | 9.06 (d, 1H), 8.55 (d, 1H), 8.24 (s, 1H), 8.19 (d, 1H), 8.11 |
| | (d, 1H), 8.03 (d, 4H), 8.00 (d, 1H), 7.92 (d, 2H), 7.82 (d, 1H), |
| | 7.62 (t, 2H), 7.59 (t, 2H), 7.58 (t, 1H), 7.55 (s, 1H), 7.52 |
| | (d, 1H), 7.50 (d, 1H), 7.40-7.32 (m, 6H), 7.20-7.11 (d, 4H) |
| 271 | 9.06 (d, 1H), 8.24 (s, 1H), 8.19 (d. 1H), 8.11(d, 1H), 8.03 |
| | (d, 4H), 8.00 (d, 1H), 7.98 (d, 1H), 7.92 (d, 2H), 7.82 (d, 1H), |
| | 7.59 (t, 2H), 7.54 (d, 1H), 7.49 (s, 1H), 7.42 (s, 1H), 7.40 |
| | (d, 1H), 7.39 (t, 1H), 7.32 (t, 4H), 7.31 (t, 1H), 7.20 (t, 1H), |
| | 7.18 (t, 1H) |
| 272 | 9.06 (d, 1H), 8.45 (d, 1H), 8.24-7.82 (m, 14H), 7.59 (t, 2H), |
| | 7.56 (t, 1H), 7.40 (d, 1H), 7.32 (t, 4H), 7.31 (t, 1H), 7.20 |
| | (t, 1H), 7.18 (t, 1H) |
| 281 | 8.32 (s, 1H), 8.03 (d, 4H), 7.99 (d, 1H), 7.67 (d, 1H), 7.59 |
| | (t, 2H), 7.55 (d, 3H), 7.38 (d, 1H), 7.32 (t, 4H), 7.27 (d, 2H), |
| | 7.24 (t, 4H), 7.08 (d, 4H), 7.00 (t, 2H) |
| 287 | 8.32 (s, 1H), 8.03 (d, 4H), 7.99 (d, 1H), 7.59-7.27 (m, 28H), |
| | 7.11 (s, 1H) |
| 297 | 8.32 (s, 1H), 8.03 (d, 4H), 7.99 (d, 1H), 7.67 (d, 1H), 7.59 |
| | (t, 2H), 7.55 (d, 3H), 7.38 (d, 1H), 7.32 (t, 4H), 7.27 (d, 2H), |
| | 7.25 (d, 4H), 7.24 (t, 4H), 7.08 (d, 4H), 7.00 (t, 2H) |
| 305 | 8.32 (s, 1H), 8.03 (d, 4H), 7.99 (d, 1H), 7.84 (d, 1H), |
| | 7.59-7.00 (m, 28H) |
| 313 | 8.45 (d, 1H), 8.32 (s, 1H), 8.03 (d, 4H), 7.99 (d, 1H), 7.86 |
| | (d, 1H), 7.74 (d, 1H), 7.67 (d, 1H), 7.64 (s, 1H), 7.59 (t, 2H), |
| | 7.56 (t, 1H), 7.55 (d, 3H), 7.43 (d, 1H), 7.38 (d, 1H), 7.32 |
| | (t, 4H), 7.31 (t, 1H), 7.27 (d, 1H), 7.24 (t, 2H) |
| 323 | 8.32 (s, 1H), 8.03 (3, 4H), 8.02 (s, 1H), 7.99 (d, 1H), 7.67 |
| | (d, 1H), 7.60 (d, 1H), 7.59 (t, 2H), 7.57 (d, 1H), 7.55 (d, 2H), |
| | 7.47 (t, 1H), 7.38 (d, 1H), 7.32 (t, 4H), 7.24 (t, 4H), 7.08 |
| | (d, 4H), 7.00 (t, 2H) |
| 336 | 8.32 (s, 1H), 8.03 (d, 4H), 7.99 (d, 1H), 7.75 (d, 2H), |
| | 7.67-7.27 (m, 20H), 7.07 (d, 2H), 7.06 (d, 2H) |
| 338 | 8.32 (s, 1H), 8.03 (d, 4H), 7.99 (d, 1H), 7.93 (d, 2H), 7.84 |
| | (d, 2H), 7.67 (d, 1H), 7.59 (t, 2H), 7.55 (d, 5H), 7.38 (d, 1H), |
| | 7.32 (t, 4H), 7.27 (d, 4H), 7.24 (t, 2H), 7.08 (d, 2H), 7.00 |
| | (t, 1H) |
| 340 | 8.32 (s, 1H), 8.03 (d, 4H), 7.99 (d, 1H), 7.67 (d, 1H), 7.59 |
| | (t, 2H), 7.55 (d, 4H), 7.38 (d, 3H), 7.32 (t, 4H), 7.30 (d, 2H), |
| | 7.27 (d, 4H), 7.24 (t, 2H), 7.08 (d, 2H), 7.00 (t, 1H) |
| 356 | 8.45 (d, 1H), 8.32 (s, 1H), 8.10 (d, 1H), 8.03 (d, 4H), 7.99 |
| | (d, 1H), 7.95 (s, 1H), 7.86 (d, 1H), 7.85 (d, 1H), 7.67 (d, 1H), |
| | 7.59 (t, 2H), 7.56 (t, 1H), 7.55 (t, 1H), 7.54 (d, 1H), 7.43 |
| | (t, 2H), 7.42 (d, 1H), 7.41 (t, 1H), 7.38 (d, 1H), 7.34 (t, 1H), |
| | 7.32 (t, 4H), 7.31 (t, 1H), 7.27 (s, 1H), 7.26 (d, 1H), 7.17 |
| | (d, 2H), 7.14 (t, 1H), 7.08 (d, 2H) |
| 376 | 8.10 (d, 1H), 8.03 (d, 4H), 7.75 (d, 2H), 7.67-7.27 (m, 20H), |
| | 7.18 (d, 1H), 7.17 (d, 1H), 7.14 (t, 1H), 7.11 (s, 1H), 7.08 |
| | (d, 2H) |
| 382 | 8.54 (d, 2H), 8.03 (d, 5H), 7.99 (d, 2H), 7.83 (s, 1H), 7.67 |
| | (d, 2H), 7.61 (t, 2H), 7.59 (t, 2H), 7.55 (d, 1H), 7.53 (t. 2H), |
| | 7.52 (d, 2H), 7.40 (d, 1H), 7.36 (d, 1H), 7.32 (t, 4H) 7.16 |
| | (d, 1H) |
| 391 | 8.19 (d, 1H), 8.03 (d, 5H), 7.98 (d, 1H), 7.83 (s, 1H), 7.67 |
| | (d, 1H), 7.59 (t, 2H), 7.55 (d, 1H), 7.54 (d, 1H), 7.49 (s, 1H), |
| | 7.42 (s, 1H), 7.40 (d, 1H), 7.39 (t, 1H), 7.36 (d, 1H), 7.32 |
| | (t, 4H), 7.31 (t, 1H), 7.20 (t, 1H), 7.18 (t, 1H) |
| 392 | 8.45 (d, 1H), 8.19 (d, 1H), 8.03 (d, 5H), 7.86 (s, 2H), 7.83 |
| | (s, 1H), 7.78 (s, 1H), 7.67 (d, 1H), 7.59 (t, 2H), 7.56 (t, 1H), |
| | 7.55 (d, 1H), 7.40 (d, 1H), 7.36 (d, 1H), 7.32 (t, 4H), 7.31 |
| | (t, 1H), 7.20 (t, 1H), 7.18 (t, 1H) |
| 396 | 8.55 (d, 2H), 8.19 (d, 1H), 8.03 (d, 5H), 7.83 (s, 1H), 7.67 |
| | (d, 2H), 7.59 (t, 2H), 7.55 (d, 2H), 7.52 (d, 2H), 7.40 (d, 1H), |
| | 7.36 (d, 1H), 7.32 (t, 4H), 7.20 (t, 1H), 7.19 (d, 1H), 7.18 |
| | (t, 1H), 7.16 (t, 2H), 7.11 (t, 2H) |
| 397 | 8.55 (d, 1H), 8.54 (d, 2H), 8.03 (d, 5H), 7.98 (d, 1H), 7.83 |
| | (s, 1H), 7.67 (d, 1H), 7.61 (t, 2H), 7.59 (t, 2H), 7.55 (d, 1H), |
| | 7.54 (d, 1H), 7.52 (d, 1H), 7.39 (t, 1H), 7.36 (d, 1H), 7.32 |
| | (t, 4H), 7.31 (t, 1H), 7.16 (t, 1H), 7.11 (t, 1H) |
| 403 | 8.04 (d, 1H), 8.03 (d, 4H), 7.71 (d, 2H), 7.67 (d, 1H), 7.62 |
| | (d, 2H), 7.59 (t, 2H), 7.56 (s, 2H), 7.55 (d, 2H), 7.54 (d, 2H), |
| | 7.45 (t, 2H), 7.42 (d, 2H), 7.38 (d, 2H), 7.32 (t, 4H), 7.27 |
| | (d, 1H), 7.11 (s, 2H) |
| 411 | 8.04 (d, 1H), 8.03 (d, 4H), 7.75 (d, 4H), 7.67 (d, 1H), 7.59 |
| | (t, 2H), 7.55 (d, 7H), 7.54 (s, 1H), 7.49 (t, 4H), 7.41 (t, 2H), |
| | 7.38 (d, 1H), 7.32 (t, 4H), 7.27 (d, 6H) |
| 424 | 8.25 (d, 2H), 8.22 (d, 1H), 8.03 (d, 5H), 7.84 (d, 1H), 7.67 |
| | (d, 1H), 7.63 (t, 3H), 7.59 (t, 2H), 7.58 (d, 1H), 7.55 (d, 3H), |
| | 7.50 (d, 2H), 7.45 (t, 1H), 7.38 (d, 1H), 7.32 (t, 4H), 7.27 |
| | (d, 2H), 7.24 (t, 2H), 7.08 (d, 2H), 7.00 (t, 1H) |
| 436 | 8.03 (d, 5H), 7.90 (d, 1H), 7.86 (d, 1H), 7.75 (d, 2H), 7.67 |
| | (d, 1H), 7.59 (t, 2H), 7.55 (d, 5H), 7.54 (s, 2H), 7.49 (t, 2H), |
| | 7.41 (t, 1H), 7.38 (d, 2H), 7.33 (d, 1H), 7.32 (t, 4H), 7.28 |
| | (t, 1H), 7.27 (d, 4H), 7.16 (d, 1H), 1.69 (s, 6H) |
| 445 | 8.04 (d, 1H), 8.03 (d, 4H), 7.98 (d, 1H), 7.75 (d, 2H), 7.67 |
| | (d, 1H), 7.59 (t, 2H), 7.55 (d, 3H), 7.55 (s, 1H), 7.54 (d, 1H), |
| | 7.53 (d, 1H), 7.49 (t, 2H), 7.41 (t, 1H), 7.39 (t, 1H), 7.38 |
| | (d, 1H), 7.32 (t, 4H), 7.31 (t, 1H), 7.27 (d, 3H), 7.24 (t, 2H), |
| | 7.00 (t, 1H) |
| 449 | 8.45 (d, 1H), 8.04 (d, 1H), 8.03 (d, 4H), 7.86 (d, 1H), 7.67 |
| | (d, 1H), 7.63 (d, 1H), 7.60 (d, 1H), 7.59 (t, 2H), 7.56 (t, 1H), |
| | 17.55 (d, 2H), 7.38 (d, H), 7.31 (t, 1H), 7.32 (t, 4H), 7.24 |
| | (t, 4H), 7.08 (d, 4H), 7.00 (t, 2H) |
| 466 | 8.03 (d, 5H), 7.75 (d, 2H), 7.67-7.32 (m, 21H), 7.07 (d, 2H), |
| | 7.06 (d, 2H) |
| 493 | 8.03 (d, 4H), 7.75 (d, 4H), 7.67 (d, 1H), 7.59 (t, 2H), 7.55 |
| | (d, 3H), 7.54 (d, 1H), 7.49 (t, 4H), 7.41 (t, 2H), 7.32 (t, 4H), |
| | 7.27 (s, 2H), 7.18 (d, 2H), 7.17 (d, 3H), 7.11 (s, 1H) |
| 497 | 8.03 (d, 4H), 7.60 (s, 1H), 7.59 (t, 2H), 7.75 (d, 4H), 7.67 |
| | (d, 1H), 7.55 (d, 1H), 7.54 (d, 1H), 7.49 (t, 4H), 7.41 (t, 2H), |
| | 7.37 (d, 2H), 7.32 (t, 4H), 7.24 (t, 2H), 7.17 (d, 1H), 7.11 |
| | (s, 1H), 7.08 (d, 2H), 7.00 (t, 1H) |
| 513 | 8.55 (d, 1H), 8.04 (d, 1H), 8.03 (d, 4H), 7.89 (s, 1H), 7.87 |
| | (d, 1H), 7.71 (d, 1H), 7.67 (d, 1H), 7.62 (t, 4H), 7.59 (t, 2H), |
| | 7.58 (t, 2H), 7.56 (d, 1H), 7.55 (s, 1H), 7.52 (d, 1H), 7.50 |
| | (d, 4H), 7.40 (d, 1H), 7.38 (d, 1H), 7.32 (t, 4H), 7.16 (t, 1H), |
| | 7.11 (T, 1H), 7.08 (d, 1H) |
| 515 | 8.55 (d, 1H), 8.03 (d, 4H), 7.98 (d, 2H), 7.93 (d, 1H), 7.83 |
| | (s, 1H), 7.67 (d, 1H), 7.59 (t, 2H), 7.55 (d, 1H), 7.54 (d, 2H), |
| | 7.52 (d, 1H), 7.39 (d, 2H), 7.36 (d, 1H), 7.32 (t, 4H), 7.31 |
| | (t, 2H), 7.16 (t, 1H), 7.11 (t, 1H) |
| 518 | 8.55 (d, 1H), 8.54 (d, 2H), 8.45 (d, 1H), 8.03 (d, 4H), 7.93 |
| | (d, 1H), 7.86 (d, 1H), 7.83 (s, 1H), 7.67 (d, 1H), 7.61 (t, 2H), |
| | 7.59 (t, 2H), 7.56 (t, 1H), 7.55 (d, 1H), 7.52 (d, 1H), 7.36 |
| | (d, 1H), 7.32 (t, 4H), 7.31 (t, 1H), 7.16 (d, 1H), 7.11 (d, 1H) |
| 1-1 | 8.28 (d, 1H), 8.13 (d, 1H), 7.86 (s, 1H), 7.84 (d, 2H), 7.83 |
| | (t, 1H), 7.81 (s, 1H), 7.80 (d, 2H), 7.79 (d, 2H), 7.75 (t, 1H), |
| | 7.65-7.41 (m, 10H) |
| 1-6 | 8.36 (d, 2H), 8.28 (d, 1H), 8.25 (d, 2H), 8.09 (d, 1H), 8.06 |
| | (d, 1H), 7.99 (d, 1H), 7.86 (s, 1H), 7.84 (d, 1H), 7.81 (s, 1H), |
| | 7.79 (d, 2H), 7.75 (t, 1H), 7.63 (t, 1H), 7.58 (t, 1H), 7.55 |
| | (s, 1H), 7.50 (t, 3H), 7.49 (s, 1H), 7.48 (t, 1H), 7.46 (t, 2H), |
| | 7.42 (s, 1H), 7.41 (t, 1H), 7.25 (d, 2H) |
| 1-10 | 9.09 (s, 1H), 8.49 (d, 1H), 8.36 (d, 2H), 8.26 (d, 1H), 8.16 |
| | (d, 1H), 8.09 (d, 1H), 8.08 (d, 1H), 8.06 (d, 1H), 8.00 (d, 1H), |
| | 7.99 (d, 1H), 7.86 (s, 1H), 7.84 (d, 1H), 7.81 (s, 1H), 7.75 |
| | (t, 1H), 7.63-7.38 (m, 12H) |
| 1-22 | 8.97 (d, 1H), 8.38 (d, 1H), 8.36 (d, 2H), 8.18 (d, 1H), 7.94 |
| | (s, 2H), 7.79 (d, 2H), 7.78 (d, 1H), 7.76 (s, 1H), 7.75 (d, 2H), |
| | 7.71 (t, 1H), 7.64 (t, 1H), 7.61 (d, 1H), 7.50 (t, 3H), 7.49 |
| | (t, 2H), 7.48 (t, 1H), 7.46 (t, 2H), 7.41 (t, 2H), 7.38 (s, 1H) |
| 1-32 | 9.09 (s, 1H), 8.97 (d, 1H), 8.49 (d, 1H), 8.18 (d, 1H), 8.16 |
| | (d, 1H), 8.09 (d, 2H), 8.08 (d, 1H), 8.00 (d, 1H), 7.99 |
| | (d, 2H), 7.83 (s, 1H), 7.64-7.61 (m, 17H) |
| 1-41 | 8.36 (d, 4H), 8.28 (d, 1H), 8.09 (d, 1H), 8.06 (d, 1H), 7.99 |
| | (d, 1H), 7.84 (t, 1H), 7.82 (d, 1H), 7.76 (d, 1H), 7.75 (t, 1H), |
| | 7.63 (t, 1H), 7.58 (t, 1H), 7.55 (s, 1H), 7.50 (t, 6H), 7.49 |
| | (s, 1H), 7.48 (t, 1H), 7.42 (s, 1H), 7.38 (t, 1H), 7.25 (d, 4H) |
| 1-44 | 8.36 (d, 2H), 8.28 (d, 1H), 8.25 (d, 2H), 8.09 (d, 1H), 8.06 |
| | (d, 1H), 7.99 (d, 1H), 7.84 (d, 1H), 7.82 (d, 1H), 7.76 (d, 1H), |
| | 7.75 (t, 1H), 7.74 (d, 2H), 7.63 (t, 1H), 7.58 (t, 1H), 7.55 |
| | (s, 1H), 7.50 (t, 3H), 7.49 (s, 1H), 7.48 (t, 3H), 7.42 (s, 1H), |
| | 7.41 (t, 1H), 7.25 (d, 2H) |
| 1-50 | 9.09 (s, 1H), 8.49 (d, 1H), 8.25 (d, 2H), 8.16 (d, 1H), 8.09 |
| | (d, 1H), 8.08 (d, 1H), 8.06 (d, 1H), 8.00 (d, 1H), 7.99 (d, 1H), |
| | 7.98 (d, 1H), 7.86 (s, 1H), 7.81 (s, 1H), 7.75 (d, 2H), |
| | 7.63-7.25 (m, 14H) |
| 1-52 | 9.09 (s, 1H), 8.49 (d, 1H), 8.36 (d, 2H), 8.16 (d, 1H), 8.09 |
| | (d, 1H), 8.08 (d, 1H), 8.06 (d, 1H), 8.00 (d, 1H), 7.99 (s, 1H), |
| | 7.78 (d, 1H), 7.76 (s, 1H), 7.63 (t, 1H), 7.61 (t, 1H), 7.60 |
| | (d, 1H), 7.58 (t, 1H), 7.57 (d, 3H), 7.55 (s, 1H), 7.47 (t, 1H) |
| 1-54 | 9.09 (s, 1H), 8.49 (d, 1H), 8.36 (d, 2H), 8.16 (d, 1H), 8.09 |
| | (d, 1H), 8.06 (d, 1H), 8.00 (d, 1H), 7.99 (d, 1H), 7.98 (d, 1H), |
| | 7.86 (s, 1H), 7.81 (s, 1H), 7.63 (t, 1H), 7.61 (t, 1H), 7.58 |
| | (t, 1H), 7.57 (t, 1H)m, 7.55 (s, 1H), 7.54 (d, 1H), 7.50 (t, 3H), |
| | 7.39-7.25 (m, 7H) |

TABLE 5

| Compound | FD-MS | Compound | FD-MS |
|---|---|---|---|
| 8 | m/z = 701.28 (C₅₂H₃₅N₃ = 701.87) | 14 | m/z = 793.35 (C₅₉H₄₃N₃ = 794.01) |
| 37 | m/z = 765.28 (C₅₆H₃₅N₃O = 765.92) | 50 | m/z = 731.24 (C₅₂H₃₃N₃S = 731.92) |
| 58 | m/z = 726.28 (C₅₃H₃₄N₄ = 726.88) | 60 | m/z = 757.35 (C₅₆H₄₃N₃ = 757.98) |
| 70 | m/z = 807.27 (C₅₈H₃₇N₃S = 808.02) | 77 | m/z = 719.40 (C₅₂H₁₇D₁₈N₃ = 719.98) |
| 95 | m/z = 731.24 (C₅₂H₃₃N₃S = 731.92) | 101 | m/z = 791.29 (C₅₈H₃₇N₃O = 791.95) |
| 120 | m/z = 826.39 (C₅₈H₁₈D₁₉N₃S = 827.13) | 121 | m/z = 625.25 (C₄₆H₃₁N₃ = 625.78) |
| 140 | m/z = 751.30 (C₅₆H₃₇N₃ = 751.93) | 147 | m/z = 751.30 (C₅₆H₃₇N₃ = 751.93) |
| 150 | m/z = 715.26 (C₅₂H₃₃N₃O = 715.86) | 155 | m/z = 741.31 (C₅₅H₃₉N₃ = 741.94) |
| 167 | m/z = 791.29 (C₅₈H₃₇N₃O = 791.95) | 174 | m/z = 741.31 (C₅₅H₃₉N₃ = 741.94) |
| 177 | m/z = 719.27 (C₅₂H₃₄FN₃ = 719.86) | 179 | m/z = 769.27 (C₅₃H₃₄F₃N₃ = 769.87) |
| 187 | m/z = 791.29 (C₅₈H₃₇N₃O = 791.95) | 199 | m/z = 731.47 (C₅₂H₅D₃₀N₃ = 732.06) |
| 203 | m/z = 665.28 (C₄₉H₃₅N₃ = 665.84) | 227 | m/z = 675.27 (C₅₀H₃₃N₃ = 675.84) |
| 237 | m/z = 701.28 (C₅₂H₃₅N₃ = 701.87) | 242 | m/z = 647.24 (C₄₈H₂₉N₃ = 647. 78) |
| 254 | m/z = 743.20 (C₅₂H₂₉N₃OS = 743.88) | 256 | m/z = 712.26 (C₅₂H₃₂N₄ = 712.86) |
| 265 | m/z = 597.22 (C₄₄H₂₇N₃ = 597.72) | 270 | m/z = 712.26 (C₅₂H₃₂N₄ = 712.86) |
| 271 | m/z = 637.22 (C₄₆H₂₇N₃O = 637.74) | 272 | m/z = 653.19 (C₄₆H₂₇N₃S = 653.80) |
| 281 | m/z = 575.24 (C₄₂H₂₉N₃ = 575.72) | 287 | m/z = 701.28 (C₅₂H₃₅N₃ = 701.87) |
| 297 | m/z = 667.30 (C₄₉H₃₇N₃ = 667.86) | 305 | m/z = 701.28 (C₅₂H₃₅N₃ = 701.87) |
| 313 | m/z = 681.22 (C₄₈H₃₁N₃S = 681.86) | 323 | m/z = 665.25 (C₄₈H₃₁N₃O = 665.80) |
| 336 | m/z = 669.26 (C₄₈H₃₂FN₃ = 669.80) | 338 | m/z = 676.26 (C₄₉H₃₂N₄ = 676.82) |
| 340 | m/z = 707.33 (C₅₂H₄₁N₃ = 707.92) | 343 | m/z = 687.25 (C₄₈H₃₁F₂N₃ = 687.79) |
| 356 | m/z = 757.26 (C₅₄H₃₅N₃S = 757.96) | 359 | m/z = 666.36 (C₄₈H₁₈D₁₅N₃ = 666.90) |
| 372 | m/z = 625.25 (C₄₆H₃₁N₃ = 625.78) | 376 | m/z = 651.27 (C₄₈H₃₃N₃ = 651.81) |
| 391 | m/z = 587.20 (C₄₂H₂₅N₃O = 587.68) | 392 | m/z = 603.18 (C₄₂H₂₅N₃S = 603.74) |
| 382 | m/z = 597.22 (C₄₄H₂₇N₃ = 597.72) | 396 | m/z = 662.25 (C₄₈H₃₀N₄ = 662.80) |
| 397 | m/z = 637.22 (C₄₆H₂₇N₃O = 637.74) | 403 | m/z = 675.27 (C₅₀H₃₃N₃ = 675.84) |
| 411 | m/z = 727.30 (C₅₄H₃₇N₃ = 727.91) | 424 | m/z = 701.28 (C₅₂H₃₅N₃ = 701.87) |
| 436 | m/z = 767.33 (C₅₇H₄₁N₃ = 767.98) | 445 | m/z = 741.28 (C₅₄H₃₅N₃O = 741.89) |
| 449 | m/z = 681.22 (C₄₈H₃₁N₃S = 681.86) | 459 | m/z = 719.25 (C₄₉H₃₂F₃N₃ = 719.81) |
| 466 | m/z = 741.28 (C₅₄H₃₅N₃O = 741.89) | 493 | m/z = 719.25 (C₄₈H₃₃N₃ = 651.81) |
| 478 | m/z = 791.47 (C₅₄HD₃₄N₃S = 792.16) | 497 | m/z = 719.25 (C₄₈H₃₃N₃ = 651.81) |
| 513 | m/z = 738.28 (C₅₄H₃₄N₄ = 738.89) | 515 | m/z = 677.21 (C₄₈H₂₇N₃O₂ = 677.76) |
| 518 | m/z = 653.19 (C₄₆H₂₇N₃S = 653.80) | | |
| 1-1 | m/z = 498.17 (C₃₆H₂₂N₂O = 498.59) | 1-6 | m/z = 651.23 (C₄₇H₂₉N₃O = 651.77) |
| 1-10 | m/z = 625.22 (C₄₅H₂₇N₃O = 625.73) | 1-22 | m/z = 601.22 (C₄₃H₂₇N₃O = 601.71) |
| 1-32 | m/z = 715.23 (C₅₁H₂₉N₃O₂ = 715.81) | 1-41 | m/z = 651.23 (C₄₇H₂₉N₃O = 651.77) |
| 1-44 | m/z = 651.23 (C₄₇H₂₉N₃O = 651.77) | 1-50 | m/z = 651.23 (C₄₇H₂₉N₃O = 651.77) |
| 1-52 | m/z = 575.20 (C₄₁H₂₅N₃O = 575.67) | 1-54 | m/z = 651.23 (C₄₇H₂₉N₃O = 651.77) |

### EXPERIMENTAL EXAMPLE

### Experimental Example 1

(1) Manufacture of Organic Light Emitting Device (Red Single Host & Mix Host)

A glass substrate on which ITO/Ag/ITO were coated as a thin film to thicknesses of 115 Å/100 Å/15 Å was ultrasonic cleaned with distilled water. When the cleaning with distilled water was finished, the substrate was ultrasonic cleaned with solvents such as acetone, methanol and isopropyl alcohol, then dried, and then subjected to UVO treatment for 5 minutes using UV in a UV cleaner. After that, the substrate was transferred to a plasma cleaner (PT), then subjected to plasma treatment under vacuum for ITO work function and residual film removal, and transferred to a thermal deposition apparatus for organic deposition.

On the ITO electrode (positive electrode), as common layers, 1,4,5,8,9,11-hexaazatriphenylenehexacarbonitrile (HAT-CN) was deposited to a thickness of 100 Å as a hole injection layer, N,N′-di-[(1-naphthyl)-N,N′-diphenyl]-1,1′-biphenyl)-4,4′-diamine (x-NPB) was deposited to a thickness of 1100 Å as a hole transport layer, N, N′-bis(3-methylphenyl)-N, N′-diphenylbenzidine (TPD) was deposited to a thickness of 800 Å as a light auxiliary layer, and 1,1-bis[(di-4-tolylamino)phenyl]cyclohexane (TAPC) was deposited to a thickness of 150 Å as an electron blocking layer.

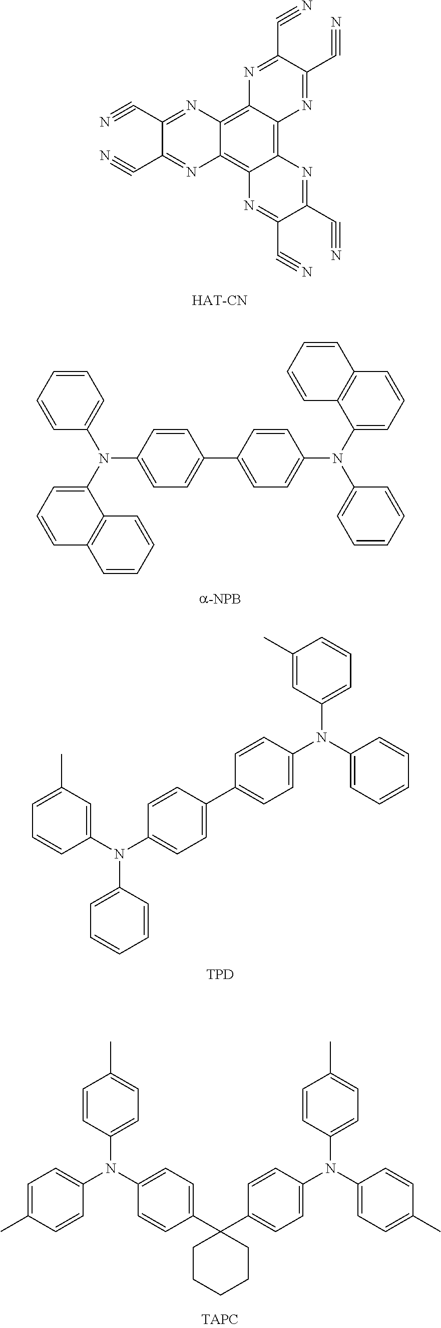

After forming the hole injection layer, the hole transport layer, the light auxiliary layer and the electron blocking layer as above, a light emitting layer was thermal vacuum deposited thereon as follows. The light emitting layer was deposited to 400 Å by depositing a single compound described in the following Table 6, or two types of compounds described in the following Table 7 in one source of supply as a red host, and, using [(piq)₂(Ir)(acac)] as a red phosphorescent dopant, doping the (piq)₂(Ir)(acac) to the host by 3 wt %.

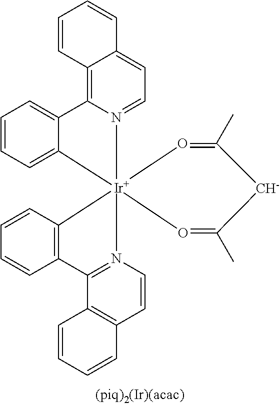

After that, bathophenanthroline (Bphen) was deposited to 30 Å as a hole blocking layer, and TPBI was deposited to 250 Å thereon as an electron transport layer.

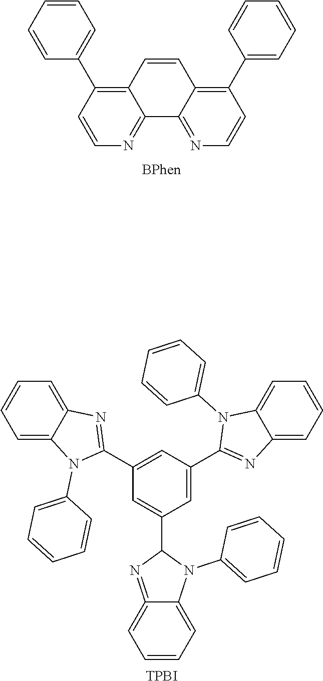

Lastly, lithium fluoride (LiF) was deposited on the electron transport layer to a thickness of 10 Å to form an electron injection layer, and then a silver (Ag) negative electrode was deposited on the electron injection layer to a thickness of 200 Å to form a negative electrode, and as a result, an organic light emitting device was manufactured. Meanwhile, all the organic compounds required to manufacture the organic light emitting device were vacuum sublimation purified under 10⁻⁸ torr to 10⁻⁶ torr for each material to be used in the manufacture of the organic light emitting device.

(2) Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, T₉₅ was measured when standard luminance was 6,000 cd/m² through a lifetime measurement system (M6000) manufactured by McScience Inc. T₉₅ means a lifetime (unit: h, hour), a time taken for luminance to become 95 with respect to initial luminance. Results of measuring driving voltage, light emission efficiency, color coordinate (CIE) and lifetime of the organic light emitting devices manufactured according to the present disclosure are shown in the following Table 6 and Table 7.

TABLE 6

| | | | Light | | |
|---|---|---|---|---|---|
| | | Driving | Emission | Color | Life- |
| | Com- | Voltage | Efficiency | Coordinate | time |
| | pound | (V) | (cd/A) | (x, y) | (T₉₅) |
| Comparative | A | 4.54 | 13.4 | (0.682, 0.317) | 16 |
| Example 1 | | | | | |
| Comparative | B | 4.55 | 12.8 | (0.682, 0.317) | 16 |
| Example 2 | | | | | |
| Comparative | C | 4.56 | 12.6 | (0.681, 0.319) | 14 |
| Example 3 | | | | | |
| Comparative | D | 4.44 | 10.6 | (0.681, 0.319) | 12 |
| Example 4 | | | | | |
| Comparative | E | 4.66 | 8.8 | (0.681, 0.319) | 10 |
| Example 5 | | | | | |
| Comparative | F | 4.48 | 10.2 | (0.681, 0.319) | 8 |
| Example 6 | | | | | |
| Comparative | G | 4.52 | 10.4 | (0.682, 0.317) | 16 |
| Example 7 | | | | | |
| Comparative | H | 4.58 | 9.2 | (0.681, 0.319) | 14 |
| Example 8 | | | | | |
| Comparative | I | 5.80 | 8.6 | (0.682, 0.317) | 12 |
| Example 9 | | | | | |
| Example 1 | 8 | 3.85 | 20.1 | (0.682, 0.317) | 85 |
| Example 2 | 14 | 3.83 | 21.8 | (0.682, 0.317) | 90 |
| Example 3 | 37 | 4.02 | 18.7 | (0.682, 0.317) | 78 |
| Example 4 | 50 | 4.08 | 18.8 | (0.682, 0.317) | 75 |
| Example 5 | 70 | 4.06 | 18.9 | (0.681, 0.319) | 77 |
| Example 6 | 77 | 3.82 | 23.8 | (0.681, 0.319) | 123 |
| Example 7 | 95 | 3.80 | 21.9 | (0.681, 0.319) | 88 |
| Example 8 | 101 | 3.78 | 23.1 | (0.682, 0.317) | 102 |
| Example 9 | 120 | 3.77 | 24.2 | (0.682, 0.317) | 134 |
| Example 10 | 121 | 3.88 | 19.1 | (0.681, 0.319) | 80 |
| Example 11 | 140 | 4.00 | 18.2 | (0.681, 0.319) | 70 |
| Example 12 | 147 | 4.02 | 17.9 | (0.682, 0.317) | 73 |
| Example 13 | 150 | 3.86 | 19.4 | (0.681, 0.319) | 82 |
| Example 14 | 155 | 3.92 | 19.5 | (0.682, 0.317) | 75 |
| Example 15 | 167 | 3.92 | 18.9 | (0.682, 0.317) | 80 |
| Example 16 | 174 | 3.93 | 18.5 | (0.682, 0.317) | 70 |
| Example 17 | 177 | 3.77 | 19.3 | (0.681, 0.319) | 72 |
| Example 18 | 187 | 3.83 | 19.4 | (0.681, 0.319) | 80 |
| Example 19 | 199 | 3.82 | 19.8 | (0.681, 0.319) | 100 |
| Example 20 | 227 | 3.80 | 19.1 | (0.681, 0.319) | 80 |
| Example 21 | 254 | 4.04 | 17.9 | (0.682, 0.317) | 69 |
| Example 22 | 256 | 4.10 | 17.0 | (0.682, 0.317) | 70 |
| Example 23 | 265 | 4.06 | 17.7 | (0.682, 0.317) | 68 |
| Example 24 | 270 | 4.06 | 17.4 | (0.682, 0.317) | 66 |
| Example 25 | 281 | 3.84 | 18.7 | (0.681, 0.319) | 77 |
| Example 26 | 287 | 3.86 | 18.6 | (0.681, 0.319) | 74 |
| Example 27 | 297 | 3.90 | 17.4 | (0.682, 0.317) | 70 |
| Example 28 | 305 | 4.00 | 18.8 | (0.681, 0.319) | 71 |
| Example 29 | 313 | 3.88 | 18.6 | (0.682, 0.317) | 80 |
| Example 30 | 323 | 3.92 | 17.6 | (0.681, 0.319) | 73 |
| Example 31 | 340 | 3.96 | 17.0 | (0.682, 0.317) | 66 |
| Example 32 | 356 | 3.86 | 19.3 | (0.682, 0.317) | 83 |
| Example 33 | 359 | 3.84 | 18.9 | (0.681, 0.319) | 95 |
| Example 34 | 376 | 3.87 | 19.1 | (0.682, 0.317) | 85 |
| Example 35 | 382 | 4.08 | 17.5 | (0.682, 0.317) | 66 |
| Example 36 | 397 | 4.10 | 17.2 | (0.681, 0.319) | 64 |
| Example 37 | 403 | 3.84 | 18.0 | (0.681, 0.319) | 71 |
| Example 38 | 411 | 3.82 | 18.6 | (0.681, 0.319) | 74 |
| Example 39 | 424 | 3.91 | 17.1 | (0.682, 0.317) | 70 |
| Example 40 | 436 | 3.94 | 18.1 | (0.682, 0.317) | 70 |
| Example 41 | 445 | 3.86 | 17.6 | (0.681, 0.319) | 75 |
| Example 42 | 449 | 3.85 | 17.4 | (0.681, 0.319) | 72 |
| Example 43 | 466 | 3.86 | 18.0 | (0.681, 0.319) | 76 |
| Example 44 | 493 | 3.88 | 19.8 | (0.682, 0.317) | 77 |
| Example 45 | 497 | 3.91 | 17.1 | (0.682, 0.317) | 71 |
| Example 46 | 513 | 4.10 | 17.3 | (0.681, 0.319) | 65 |
| Example 47 | 518 | 4.10 | 17.0 | (0.681, 0.319) | 63 |
| Example 48 | 336 | 3.76 | 19.0 | (0.681, 0.319) | 70 |
| Example 49 | 338 | 3.83 | 18.3 | (0.681, 0.319) | 67 |
| Example 50 | 459 | 3.82 | 18.2 | (0.681, 0.319) | 65 |
| Example 51 | 478 | 3.83 | 19.1 | (0.681, 0.319) | 99 |
| Example 52 | 396 | 4.00 | 17.4 | (0.681, 0.319) | 64 |
| Example 53 | 391 | 4.04 | 17.2 | (0.681, 0.319) | 68 |
| Example 54 | 392 | 4.07 | 17.0 | (0.681, 0.319) | 66 |
| Example 55 | 515 | 4.07 | 17.4 | (0.682, 0.317) | 62 |
| Example 56 | 58 | 3.80 | 18.6 | (0.681, 0.319) | 69 |
| Example 57 | 179 | 3.80 | 18.1 | (0.681, 0.319) | 64 |
| Example 58 | 60 | 3.94 | 17.6 | (0.682, 0.317) | 67 |
| Example 59 | 242 | 4.00 | 17.7 | (0.682, 0.317) | 69 |
| Example 60 | 271 | 4.02 | 17.6 | (0.681, 0.319) | 70 |
| Example 61 | 272 | 4.05 | 17.3 | (0.681, 0.319) | 68 |

TABLE 7

| | | | | | Light | | |
|---|---|---|---|---|---|---|---|
| | | | | Dri- | Emsi- | | |
| | | | | ving | sion | | |
| | | Sec- | | Volt- | Effi- | Color | Life |
| | First | ond | Ratio | age | ciency | Coordinate | time |
| | Host | Host | (N:P) | (V) | (cd/A) | (x, y) | (T₉₅) |
| Com- | A | 1-1 | 1:1 | 4.30 | 15.4 | (0.681, 0.319) | 40 |
| parative | | | | | | | |
| Example 10 | | | | | | | |
| Example 62 | 281 | 1-1 | 1:1 | 3.44 | 28.7 | (0.681, 0.319) | 117 |
| Com- | I | 1-6 | 1:1 | 5.41 | 14.6 | (0.681, 0.319) | 50 |
| parative | | | | | | | |
| Example 11 | | | | | | | |
| Example 63 | 101 | 1-6 | 1:1 | 3.38 | 33.0 | (0.682, 0.317) | 142 |
| Example 64 | 449 | 1-10 | 1:2 | 3.47 | 27.4 | (0.681, 0.319) | 110 |
| Example 65 | 50 | 1-10 | 1:2 | 3.68 | 28.8 | (0.682, 0.317) | 113 |
| Com- | H | 1-22 | 1:2 | 4.18 | 17.6 | (0.681, 0.319) | 51 |
| parative | | | | | | | |
| Example 12 | | | | | | | |
| Example 66 | 265 | 1-22 | 1:3 | 3.66 | 27.6 | (0.681, 0.319) | 108 |
| Example 67 | 14 | 1-22 | 1:3 | 3.43 | 32.0 | (0.682, 0.317) | 130 |
| Example 68 | 177 | 1-32 | 1:3 | 3.38 | 28.3 | (0.682, 0.317) | 103 |
| Example 69 | 466 | 1-41 | 1:3 | 3.46 | 28.0 | (0.681, 0.319) | 116 |
| Com- | G | 1-41 | 1:2 | 4.11 | 20.1 | (0.681, 0.319) | 57 |
| parative | | | | | | | |
| Example 13 | | | | | | | |
| Example 70 | 77 | 1-41 | 1:2 | 3.41 | 34.0 | (0.681, 0.319) | 173 |
| Example 71 | 167 | 1-44 | 1:1 | 3.52 | 28.9 | (0.682, 0.317) | 119 |
| Example 72 | 411 | 1-44 | 1:1 | 3.40 | 28.8 | (0.682, 0.317) | 113 |
| Example 73 | 382 | 1-44 | 1:1 | 3.67 | 27.5 | (0.681, 0.319) | 108 |
| Example 74 | 270 | 1-32 | 1:1 | 3.65 | 27.7 | (0.681, 0.319) | 106 |
| Example 75 | 8 | 1-54 | 2:1 | 3.44 | 30.3 | (0.681, 0.319) | 125 |
| Example 76 | 493 | 1-54 | 2:1 | 3.42 | 29.7 | (0.682, 0.317) | 116 |
| Com- | F | 1-54 | 1:3 | 4.06 | 20.0 | (0.682, 0.317) | 46 |
| parative | | | | | | | |
| Example 14 | | | | | | | |
| Example 77 | 313 | 1-50 | 1:3 | 3.48 | 28.4 | (0.681, 0.319) | 123 |
| Example 78 | 95 | 1-50 | 1:1 | 3.40 | 31.9 | (0.681, 0.319) | 130 |
| Example 79 | 199 | 1-52 | 1:2 | 3.41 | 29.8 | (0.682, 0.317) | 142 |
| Example 80 | 436 | 1-52 | 1:3 | 3.51 | 28.3 | (0.682, 0.317) | 111 |
| Example 81 | 359 | 1-6 | 1:3 | 3.43 | 29.1 | (0.682, 0.317) | 134 |
| Example 82 | 287 | 1-6 | 1:1 | 3.46 | 28.8 | (0.681, 0.319) | 112 |

Table 6 shows examples of using a single host material, and Table 7 shows examples of using the compound having a favorable hole transport ability (donor (p-host)) among the compounds of the present disclosure as a first host and using the compound having a favorable electron transport ability (acceptor (n-host)) among the compounds of the present disclosure as a second host, and depositing the two host compounds in one source of supply.

Herein, Comparative Compounds A to I used in Comparative Examples 1 to 9 are as follows:

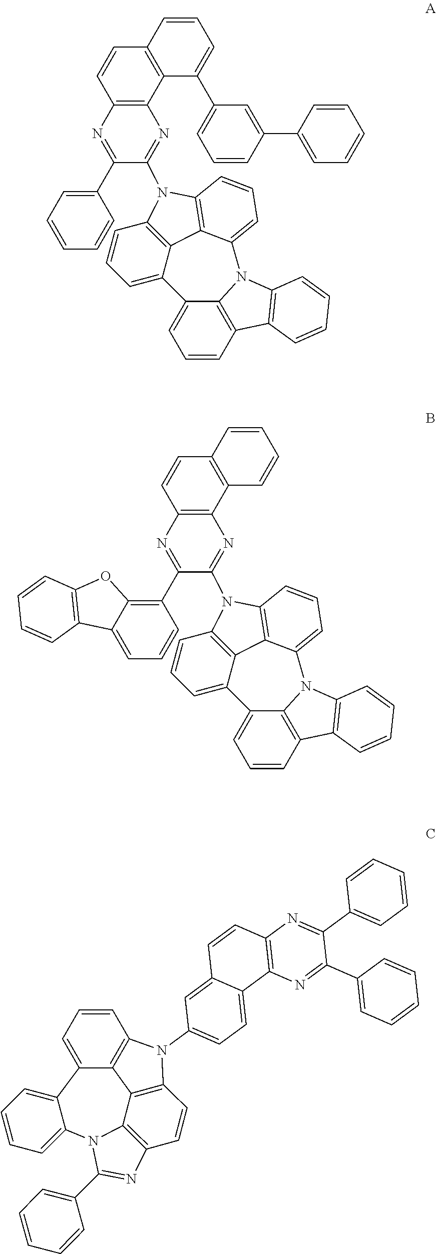 [SMILES: c1ccc(-c2cccc(-c3cccc4ccc5nc(-c6ccccc6)c(-n6c7cccc8c9cccc%10c%11ccccc%11n(c%11cccc6c%11c87)c9%10)nc5c34)c2)cc1 ; c1ccc2c(c1)ccc1nc(-c3cccc4c3oc3ccccc34)c(-n3c4cccc5c6cccc7c8ccccc8n(c8cccc3c8c54)c67)nc12 ; c1ccc(-c2nc3ccc4cc(-n5c6cccc7c8ccccc8n8c(-c9ccccc9)nc9ccc5c(c76)c98)ccc4c3nc2-c2ccccc2)cc1]

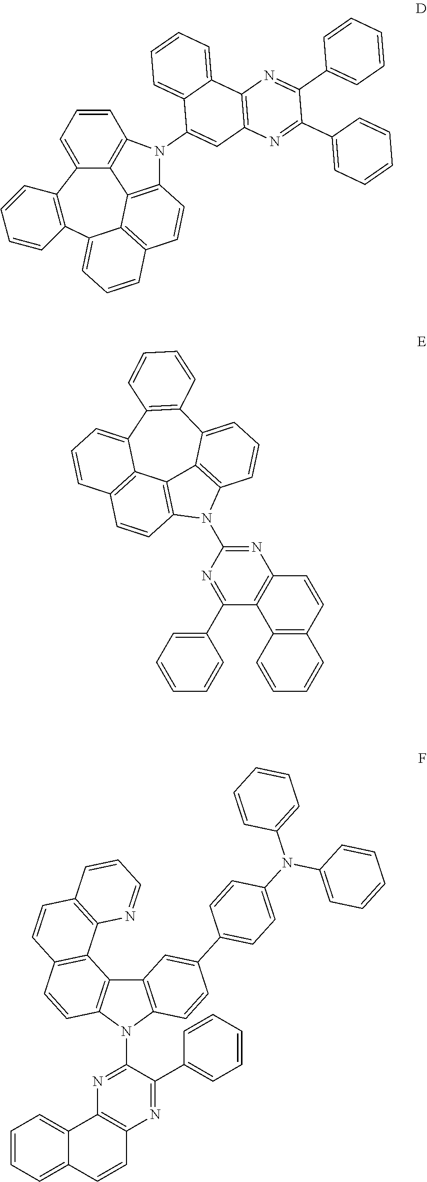 [SMILES: c1ccc(-c2nc3cc(-n4c5cccc6c5c5c7c(cccc7ccc54)-c4ccccc4-6)c4ccccc4c3nc2-c2ccccc2)cc1 ; c1ccc(-c2nc(-n3c4cccc5c4c4c6c(cccc6ccc43)-c3ccccc3-5)nc3ccc4ccccc4c23)cc1 ; c1ccc(-c2nc3ccc4ccccc4c3nc2-n2c3ccc(-c4ccc(N(c5ccccc5)c5ccccc5)cc4)cc3c3c4c(ccc5cccnc54)ccc32)cc1]

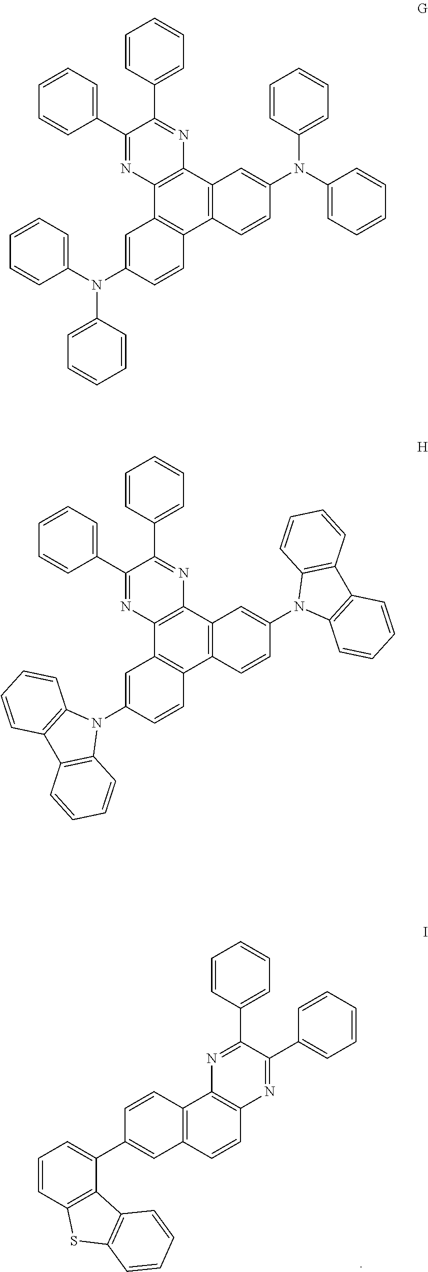 [SMILES: c1ccc(-c2nc3c4cc(N(c5ccccc5)c5ccccc5)ccc4c4ccc(N(c5ccccc5)c5ccccc5)cc4c3nc2-c2ccccc2)cc1 ; c1ccc(-c2nc3c4cc(-n5c6ccccc6c6ccccc65)ccc4c4ccc(-n5c6ccccc6c6ccccc65)cc4c3nc2-c2ccccc2)cc1 ; c1ccc(-c2nc3ccc4cc(-c5cccc6sc7ccccc7c56)ccc4c3nc2-c2ccccc2)cc1]

From the results of Tables 6 and 7, it can be seen that, when the heterocyclic compound of the present disclosure has an arylamine having a more favorable hole transport ability as a donor, that is, having a greater donating strength, as a substituent, the balance effect with an acceptor increases, resulting in a high HOMO level, thereby further reducing the driving voltage and increasing the lifetime, and accordingly, the heterocyclic compound of the present disclosure is suitable as a red host of an organic light emitting device.

In addition, compared to the compound of the present application, Comparative Compound I does not have an arylamine or carbazole with strong donor properties substituted therein. When having an amine group, a donor having a favorable hole transport ability, as a substituent, hole mobility increases due to the high HOMO level, and an electron blocking effect is obtained. Accordingly, when using the heterocyclic compound of the present disclosure as a hole transport layer material, there is an effect of lowering the driving voltage, and when using the heterocyclic compound of the present disclosure as a light emitting layer material, efficiency is improved through the effects of reducing current leakage and confining electrons. As seen from Tables 6 and 7, the organic light emitting devices using Comparative Compounds A to I in the organic material layer have reduced efficiency and lifetime.

In addition, it can be identified that, although Comparative Compounds A to F have a reduced driving voltage as seen from Tables 6 and 7, lifetime and efficiency are low despite the substitution with an arylamine having strong donor properties. It can be identified that this is due to the fact that the T1 level is high depending on the substituent position of the heterocyclic compound core, making it difficult to transfer energy to the red dopant, and resulting in large resistance due to the large band gap, and it can be identified that the lifetime is reduced since stability of the organic light emitting device decreases.

In addition, Comparative Compounds A to E have poor thermal stability when substituted with a spiro-type fused carbazole, which affects a decrease in the lifetime of the device.

Comparative Compounds G and H are not amorphous and have improved crystallinity when the symmetric core is symmetrically substituted with a donor, and this generates deterioration in the substrate due to small morphology, and the lifetime of the device is reduced.

Accordingly, it can be identified that the compound of the present application has a reduced band gap and a smaller T1 level, thereby capable of significantly improving lifetime and efficiency.

In addition, as seen from Table 7, it can be identified that driving voltage, efficiency and lifetime of the organic light emitting device may be improved when the heterocyclic compound represented by Chemical Formula 1 and the heterocyclic compound of Chemical Formula A are included in the organic material layer of the organic light emitting device at the same time.

Such a result may lead to a forecast that an exciplex phenomenon occurs when the two compounds are included at the same time. The exciplex phenomenon is a phenomenon of releasing energy having sizes of a donor (p-host) HOMO level and an acceptor (n-host) LUMO level due to electron exchanges between a molecule having strong donor properties and a molecule having strong acceptor properties. When a donor (p-host) having a favorable hole transport ability and an acceptor (n-host) having a favorable electron transport ability are used as a host of a light emitting layer, holes are injected to the p-host and electrons are injected to the n-host, and therefore, a driving voltage may be lowered, which resultantly may help with enhancement in the lifetime. When the exciplex phenomenon occurs between two molecules as above, reverse intersystem crossing (RISC) occurs, and as a result, internal quantum efficiency of fluorescence may increase up to 100%.

### Experimental Example 2

(1) Manufacture of Organic Light Emitting Device (2 Stack Red N+P Mixed Host)

A glass substrate on which indium tin oxide (ITO) was coated as a thin film to a thickness of 1,500 Å was ultrasonic cleaned with distilled water. When the cleaning with distilled water was finished, the substrate was ultrasonic cleaned with solvents such as acetone, methanol and isopropyl alcohol, then dried, and then subjected to ultraviolet ozone (UVO) treatment for 5 minutes using ultraviolet (UV) in a UV cleaner. After that, the substrate was transferred to a plasma cleaner (PT), then subjected to plasma treatment under vacuum for ITO work function and residual film removal, and transferred to a thermal deposition apparatus for organic deposition.

On the transparent ITO electrode (positive electrode), as common layers, 4,4′,4″-tris[2-naphthyl(phenyl)amino] triphenylamine (2-TNATA) was deposited as a hole injection layer, and N, N′-di(1-naphthyl)-N, N′-diphenyl-(1,1′-biphenyl)-4,4′-diamine (NPB) was deposited as a hole transport layer.

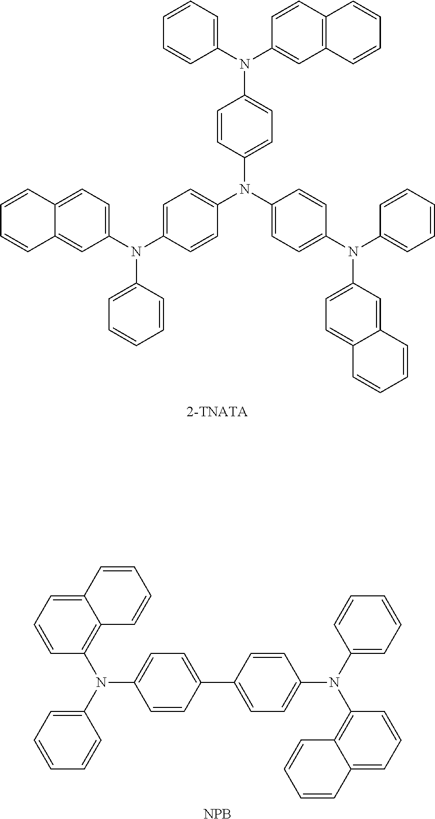 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transport layer as above, a light emitting layer was thermal vacuum deposited thereon as follows. The light emitting layer was deposited to a thickness of 400 Å using compounds of Chemical Formulae 1 and 2 described in the following Table 8 as a red host and, using (piq)₂(Ir)(acac) as a red phosphorescent dopant, doping the (piq)₂ (Ir)(acac) to the host by 2 wt %.

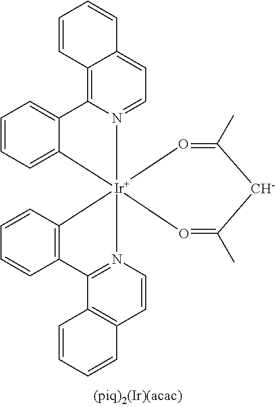

After that, tris(8-hydroxyquinolinato)aluminum (Alq₃) was deposited to a thickness of 120 Å as an electron transport layer, bathophenanthroline (Bphen) was deposited to a thickness of 120 Å thereon as a charge generation layer, and molybdenum trioxide (MoO₃) was deposited to a thickness of 100 Å thereon as a charge generation layer as well.

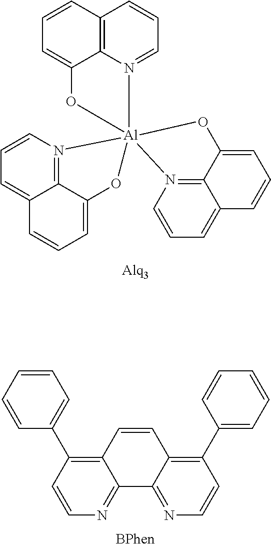

After that, N, N′-di(1-naphthyl)-N, N′-diphenyl-(1,1′-biphenyl)-4, 4′-diamine (NPB) was deposited to a thickness of 420 Å as a hole transport layer.

A light emitting layer was thermal vacuum deposited thereon as follows. The light emitting layer was deposited to 400 Å using compounds of Chemical Formulae 1 and 2 described in the following Table 8 as a red host and, using (piq)₂(Ir)(acac) as a red phosphorescent dopant, doping the (piq)₂(Ir)(acac) to the host by 2 wt %.

After that, tris(8-hydroxyquinolinato)aluminum (Alq₃) was deposited to a thickness of 300 Å as an electron transport layer.

Lastly, lithium fluoride (LiF) was deposited on the electron transport layer to a thickness of 20 Å to form an electron injection layer, and then an aluminum (Al) negative electrode was deposited on the electron injection layer to a thickness of 1,200 Å to form a negative electrode, and as a result, an organic light emitting device was manufactured. Meanwhile, all the organic compounds required to manufacture the organic light emitting device were vacuum sublimation purified under 10⁻⁸ torr to 10⁻⁶ torr for each material to be used in the manufacture of the organic light emitting device.

For each of the organic light emitting devices manufactured as above, electroluminescent (EL) properties were measured using M7000 manufactured by McScience Inc., and with the measurement results, T₉₅ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are shown in the following Table 8.

TABLE 8

| | | | | | Light | | |
|---|---|---|---|---|---|---|---|
| | | | | Dri- | Emis- | | |
| | | | | ving | sion | | |
| | | Sec- | | Volt- | Effi- | Color | Life |
| | First | ond | Ratio | age | ciency | Coordinate | time |
| | Host | Host | (N:P) | (V) | (cd/A) | (x, y) | (T₉₅) |
| Com- | A | 1-1 | 1:1 | 7.14 | 30.3 | (0.681, 0.319) | 79 |
| parative | | | | | | | |
| Example 15 | | | | | | | |
| Example 83 | 281 | 1-1 | 1:1 | 5.71 | 56.5 | (0.681, 0.319) | 230 |
| Com- | I | 1-6 | 1:1 | 8.98 | 28.8 | (0.681, 0.319) | 99 |
| parative | | | | | | | |
| Example 16 | | | | | | | |
| Example 84 | 101 | 1-6 | 1:1 | 5.61 | 65.0 | (0.682, 0.317) | 280 |
| Example 85 | 449 | 1-10 | 1:2 | 5.76 | 54.0 | (0.681, 0.319) | 217 |
| Example 86 | 50 | 1-10 | 1:2 | 6.11 | 56.7 | (0.682, 0.317) | 223 |
| Com- | H | 1-22 | 1:2 | 6.94 | 34.7 | (0.681, 0.319) | 100 |
| parative | | | | | | | |
| Example 17 | | | | | | | |
| Example 87 | 265 | 1-22 | 1:3 | 6.08 | 54.4 | (0.681, 0.319) | 213 |
| Example 88 | 14 | 1-22 | 1:3 | 5.69 | 63.0 | (0.682, 0.317) | 256 |
| Example 89 | 177 | 1-32 | 1:3 | 5.61 | 55.8 | (0.682, 0.317) | 203 |
| Example 90 | 466 | 1-41 | 1:3 | 5.74 | 55.2 | (0.681, 0.319) | 229 |
| Com- | G | 1-41 | 1:2 | 6.82 | 39.6 | (0.681, 0.319) | 112 |
| parative | | | | | | | |
| Example 18 | | | | | | | |
| Example 91 | 77 | 1-41 | 1:2 | 5.66 | 67.0 | (0.681, 0.319) | 341 |
| Example 92 | 167 | 1-44 | 1:1 | 5.84 | 56.9 | (0.682, 0.317) | 234 |
| Example 93 | 411 | 1-44 | 1:1 | 5.64 | 56.7 | (0.682, 0.317) | 223 |
| Example 94 | 382 | 1-44 | 1:1 | 6.09 | 54.2 | (0.681, 0.319) | 213 |
| Example 95 | 270 | 1-32 | 1:1 | 6.06 | 54.6 | (0.681, 0.319) | 209 |
| Example 96 | 8 | 1-54 | 2:1 | 5.71 | 59.7 | (0.681, 0.319) | 246 |
| Example 97 | 493 | 1-54 | 2:1 | 5.68 | 58.5 | (0.682, 0.317) | 229 |
| Com- | F | 1-54 | 1:3 | 6.74 | 39.4 | (0.682, 0.317) | 91 |
| parative | | | | | | | |
| Example 19 | | | | | | | |
| Example 98 | 313 | 1-50 | 1:3 | 5.78 | 55.9 | (0.681, 0.319) | 242 |
| Example 99 | 95 | 1-50 | 1:1 | 5.64 | 62.8 | (0.681, 0.319) | 256 |
| Example | 199 | 1-52 | 1:2 | 5.66 | 58.7 | (0.682, 0.317) | 280 |
| 100 | | | | | | | |
| Example | 436 | 1-52 | 1:3 | 5.83 | 55.8 | (0.682, 0.317) | 219 |
| 101 | | | | | | | |
| Example | 359 | 1-6 | 1:3 | 5.69 | 57.3 | (0.682, 0.317) | 264 |
| 102 | | | | | | | |
| Example | 287 | 1-6 | 1:1 | 5.74 | 56.7 | (0.681, 0.319) | 221 |
| 103 | | | | | | | |

From the results of Table 8, it was identified that, when manufacturing the organic light emitting device by laminating the light emitting layer including both the compound represented by Chemical Formula 1 and the compound represented by Chemical Formula A of the present disclosure as two stacks, the light emitting layers were deposited twice, increasing efficiency compared to when manufactured as a single stack.

Meanwhile, like the organic light emitting device manufactured as a single stack as in Experimental Example 1, the organic light emitting device including two stacks was capable of, by including the compound represented by Chemical Formula A of the present disclosure in the organic material layer of the organic light emitting device, significantly improving the driving voltage due to an increase in the hole mobility. In addition, it was identified that efficiency was improved as well by reducing current leakage through electron blocking, and confining electrons.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

What is claimed is:

1. A heterocyclic compound represented by the following Chemical Formula 1: 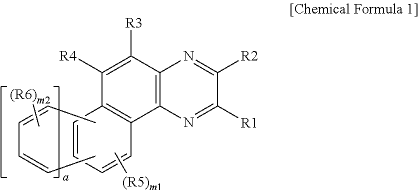
wherein, in Chemical Formula 1,
a is an integer of 0 or 1;
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; a group represented by the following Chemical Formula 2-1; and a group represented by the following Chemical Formula 2-2;
at least one of R5 and R6 is the group represented by the following Chemical Formula 2-1; or the group represented by the following Chemical Formula 2-2; and
m1 and m2 are each independently one of integers of 1 to 4, and when a is 1, m1 is an integer of 1 or 2, 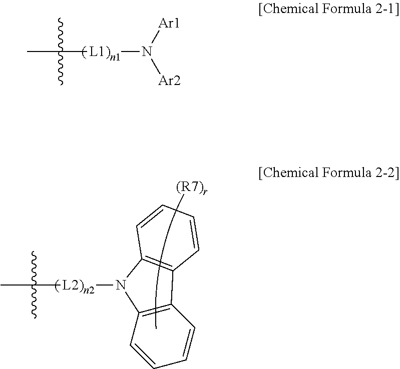
in Chemical Formula 2-1 or Chemical Formula 2-2,
Ar1 and Ar2 are the same as or different from each other, and each independently a substituted or unsubstituted C6 to C60 aryl group; or a substituted or unsubstituted C2 to C60 heteroaryl group;
R7s are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heteroring;
r is one of integers of 1 to 8;
L1 and L2 are the same as or different from each other, and each independently a single bond; a substituted or unsubstituted C6 to C60 arylene group; or a substituted or unsubstituted C2 to C60 heteroarylene group; and
n1 and n2 are the same as or different from each other, and each independently one of integers of 0 to 3, and when n1 and n2 are 2 or greater, L1s or L2s are the same as or different from each other.

2. The heterocyclic compound of claim 1, wherein Chemical Formula 1 is represented by the following Chemical Formula 1-a or Chemical Formula 1-b: 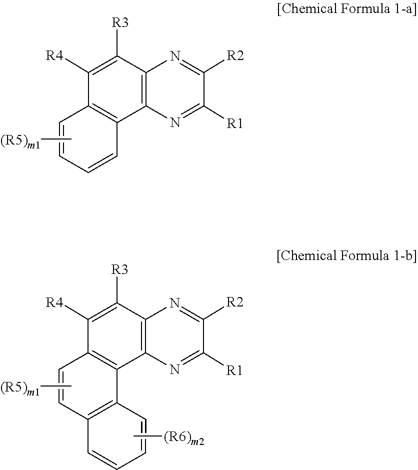
in Chemical Formula 1-a and Chemical Formula 1-b,
R1 to R6, m1 and m2 have the same definitions as in Chemical Formula 1.

3. The heterocyclic compound of claim 1, wherein Chemical Formula 1 is represented by the following Chemical Formula 1-a-1 or Chemical Formula 1-b-1: 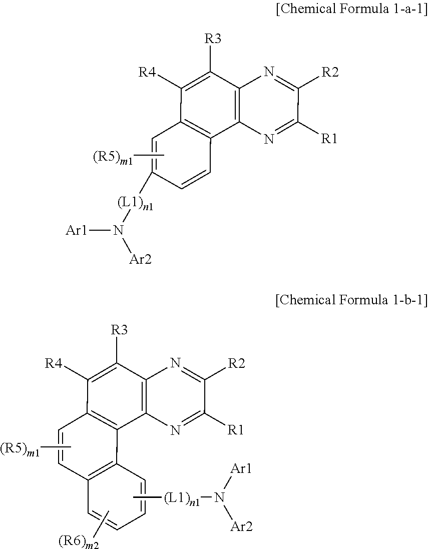
in Chemical Formula 1-a-1 and Chemical Formula 1-b-1,
R1 to R6, L1, Ar1, Ar2, n1, m1 and m2 have the same definitions as in Chemical Formula 1.

4. The heterocyclic compound of claim 1, wherein Chemical Formula 1 is represented by the following Chemical Formula 1-a-2 or Chemical Formula 1-b-2: 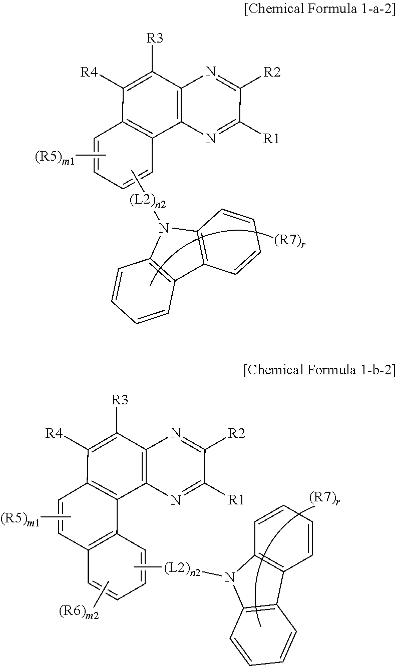
in Chemical Formula 1-a-2 and Chemical Formula 1-b-2,
R1 to R7, L2, n2, m1, m2 and r have the same definitions as in Chemical Formula 1.

5. The heterocyclic compound of claim 1, wherein Chemical Formula 1 is represented by any one of the following Chemical Formula 1-a-1-1, Chemical Formula 1-a-1-2, Chemical Formula 1-a-2-1, Chemical Formula 1-a-2-2, Chemical Formula 1-b-1-1, Chemical Formula 1-b-1-2, Chemical Formula 1-b-2-1 and Chemical Formula 1-b-2-2: 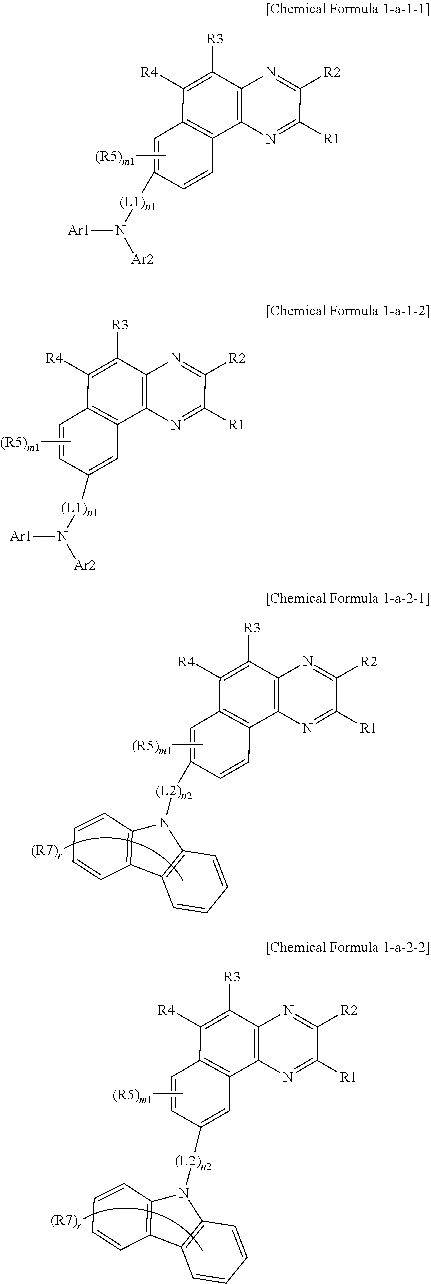 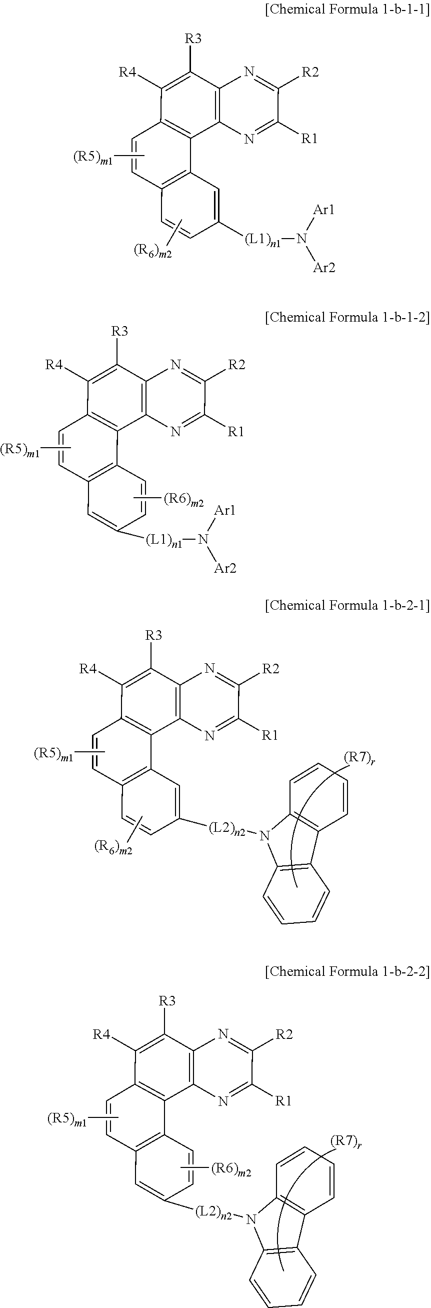
in Chemical Formula 1-a-1-1, Chemical Formula 1-a-1-2, Chemical Formula 1-a-2-1, Chemical Formula 1-a-2-2, Chemical Formula 1-b-1-1, Chemical Formula 1-b-1-2, Chemical Formula 1-b-2-1 and Chemical Formula 1-b-2-2,
R1 to R7, L1, L2, Ar1, Ar2, n1, n2, m1, m2 and r have the same definitions as in Chemical Formula 1.

6. The heterocyclic compound of claim 1, wherein R1 and R2 are the same as or different from each other, and each independently a substituted or unsubstituted C6 to C60 aryl group.

7. The heterocyclic compound of claim 1, wherein the group represented by Chemical Formula 2-2 is any one of groups represented by the following Chemical Formula 2-2-a, Chemical Formula 2-2-b, Chemical Formula 2-2-c and Chemical Formula 2-2-d: 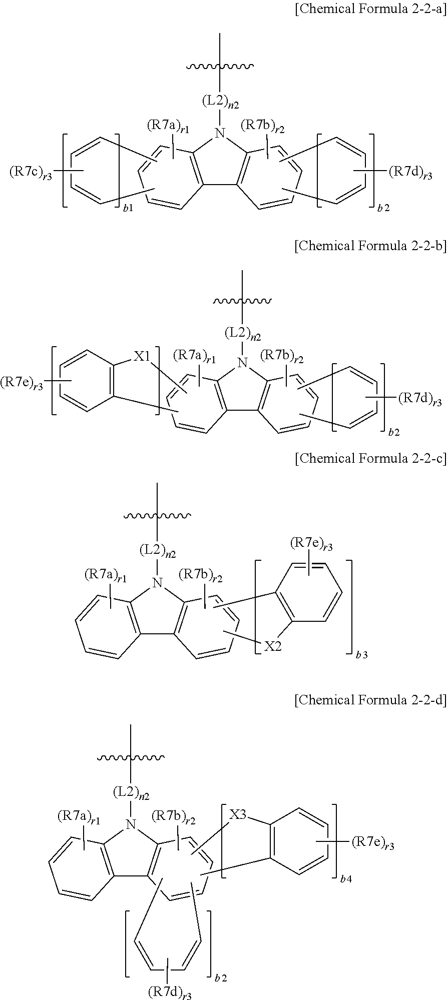
in Chemical Formulae 2-2-a to 2-2-d,
L2 and n2 have the same definitions as in Chemical Formula 1;
R7a to R7e are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C6 to C60 aryl group; and a substituted or unsubstituted C2 to C60 heteroaryl group;
X1 to X3 are the same as or different from each other and each independently NR′, O or S, and herein, R′ is a substituted or unsubstituted C6 to C60 aryl group; or a substituted or unsubstituted C2 to C60 heteroaryl group;
b1, b2 and b3 are the same as or different from each other, and each independently an integer of 0 or 1;
b4 is an integer of 0, 1 or 2, however, when b4 is 2, b2 is 0; and
r1, r2 and r3 are the same as or different from each other, and each independently one of integers of 0 to 4.

8. The heterocyclic compound of claim 1, wherein the heterocyclic compound represented by Chemical Formula 1 does not include deuterium as a substituent, or has a deuterium content of 1% to 100% based on a total number of hydrogen atoms and deuterium atoms.

9. The heterocyclic compound of claim 1, wherein the heterocyclic compound represented by Chemical Formula 1 is any one selected from the group consisting of the following compounds:

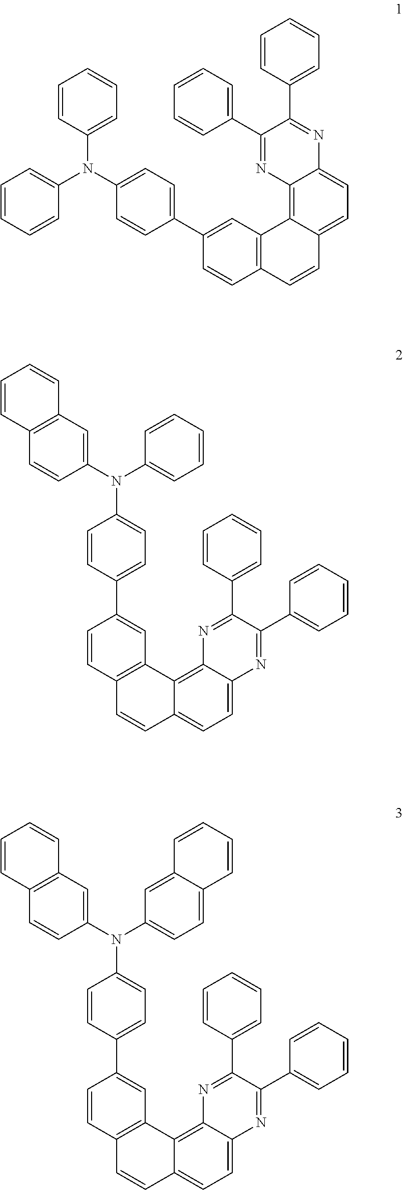 [SMILES: c1ccc(-c2nc3ccc4ccc5ccc(-c6ccc(N(c7ccccc7)c7ccccc7)cc6)cc5c4c3nc2-c2ccccc2)cc1 ; c1ccc(-c2nc3ccc4ccc5ccc(-c6ccc(N(c7ccccc7)c7ccc8ccccc8c7)cc6)cc5c4c3nc2-c2ccccc2)cc1 ; c1ccc(-c2nc3ccc4ccc5ccc(-c6ccc(N(c7ccc8ccccc8c7)c7ccc8ccccc8c7)cc6)cc5c4c3nc2-c2ccccc2)cc1]

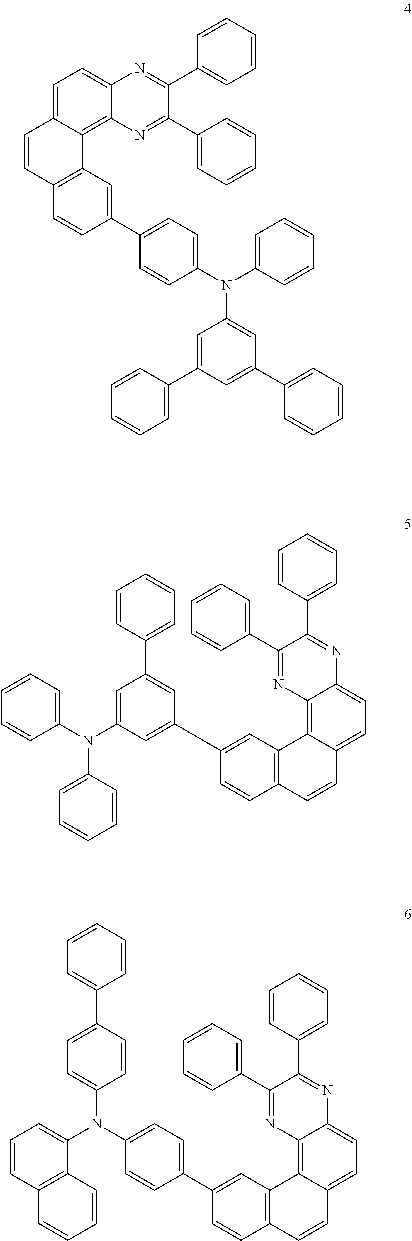 [SMILES: c1ccc(-c2cc(-c3ccccc3)cc(N(c3ccccc3)c3ccc(-c4ccc5ccc6ccc7nc(-c8ccccc8)c(-c8ccccc8)nc7c6c5c4)cc3)c2)cc1 ; c1ccc(-c2cc(-c3ccc4ccc5ccc6nc(-c7ccccc7)c(-c7ccccc7)nc6c5c4c3)cc(N(c3ccccc3)c3ccccc3)c2)cc1 ; c1ccc(-c2ccc(N(c3ccc(-c4ccc5ccc6ccc7nc(-c8ccccc8)c(-c8ccccc8)nc7c6c5c4)cc3)c3cccc4ccccc34)cc2)cc1]

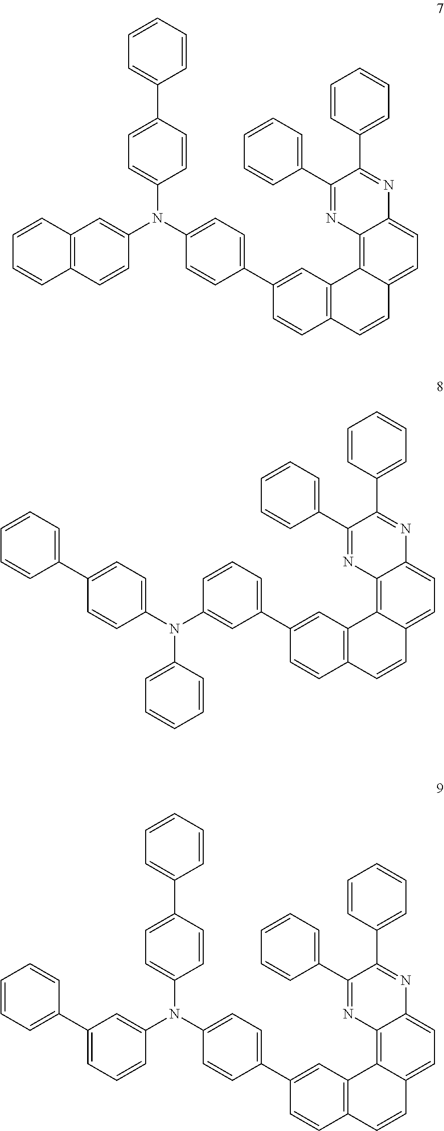 [SMILES: c1ccc(-c2ccc(N(c3ccc(-c4ccc5ccc6ccc7nc(-c8ccccc8)c(-c8ccccc8)nc7c6c5c4)cc3)c3ccc4ccccc4c3)cc2)cc1 ; c1ccc(-c2ccc(N(c3ccccc3)c3cccc(-c4ccc5ccc6ccc7nc(-c8ccccc8)c(-c8ccccc8)nc7c6c5c4)c3)cc2)cc1 ; c1ccc(-c2ccc(N(c3ccc(-c4ccc5ccc6ccc7nc(-c8ccccc8)c(-c8ccccc8)nc7c6c5c4)cc3)c3cccc(-c4ccccc4)c3)cc2)cc1]

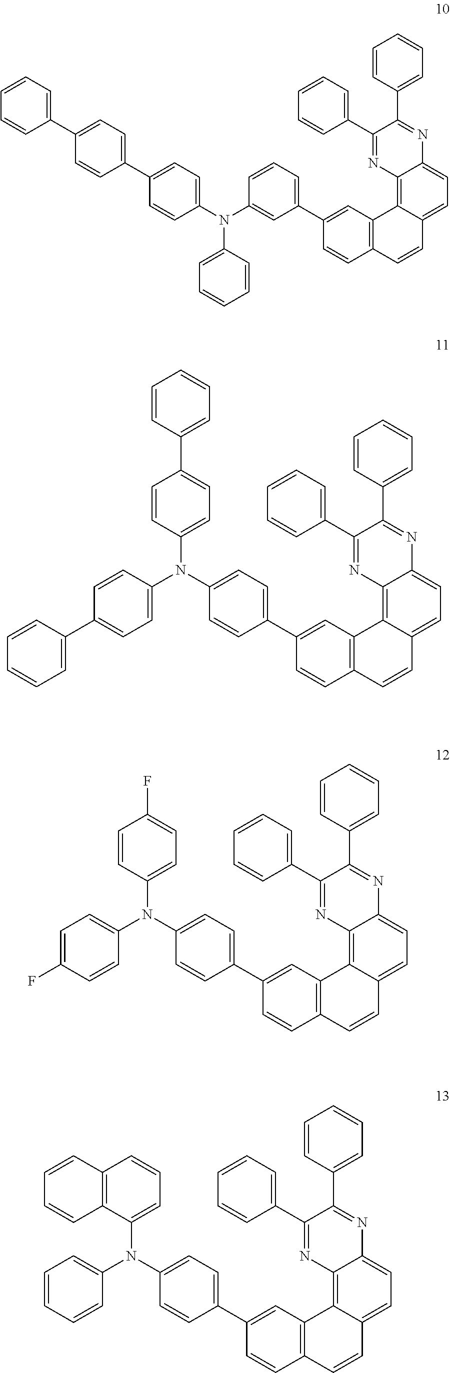 [SMILES: c1ccc(-c2ccc(-c3ccc(N(c4ccccc4)c4cccc(-c5ccc6ccc7ccc8nc(-c9ccccc9)c(-c9ccccc9)nc8c7c6c5)c4)cc3)cc2)cc1 ; c1ccc(-c2ccc(N(c3ccc(-c4ccccc4)cc3)c3ccc(-c4ccc5ccc6ccc7nc(-c8ccccc8)c(-c8ccccc8)nc7c6c5c4)cc3)cc2)cc1 ; Fc1ccc(N(c2ccc(F)cc2)c2ccc(-c3ccc4ccc5ccc6nc(-c7ccccc7)c(-c7ccccc7)nc6c5c4c3)cc2)cc1 ; c1ccc(-c2nc3ccc4ccc5ccc(-c6ccc(N(c7ccccc7)c7cccc8ccccc78)cc6)cc5c4c3nc2-c2ccccc2)cc1]

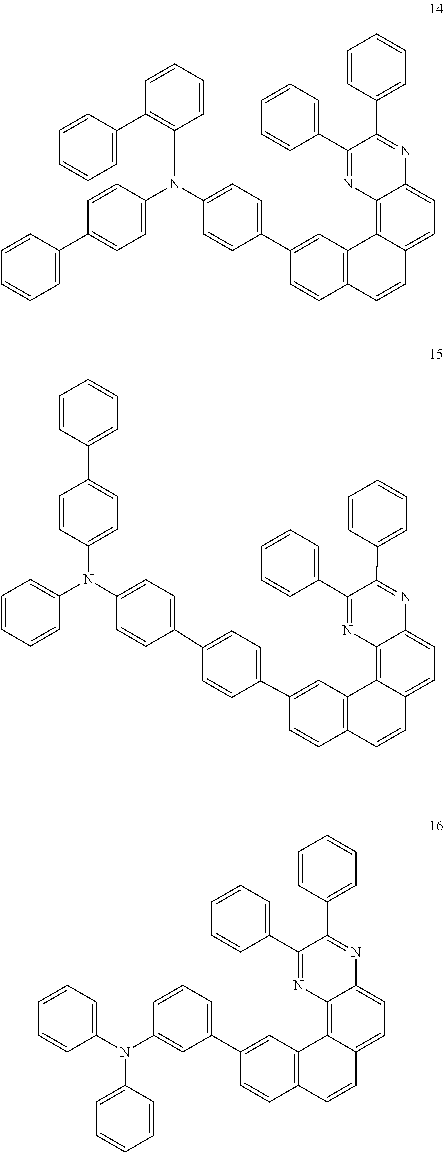 [SMILES: c1ccc(-c2ccc(N(c3ccc(-c4ccc5ccc6ccc7nc(-c8ccccc8)c(-c8ccccc8)nc7c6c5c4)cc3)c3ccccc3-c3ccccc3)cc2)cc1 ; c1ccc(-c2ccc(N(c3ccccc3)c3ccc(-c4ccc(-c5ccc6ccc7ccc8nc(-c9ccccc9)c(-c9ccccc9)nc8c7c6c5)cc4)cc3)cc2)cc1 ; c1ccc(-c2nc3ccc4ccc5ccc(-c6cccc(N(c7ccccc7)c7ccccc7)c6)cc5c4c3nc2-c2ccccc2)cc1]

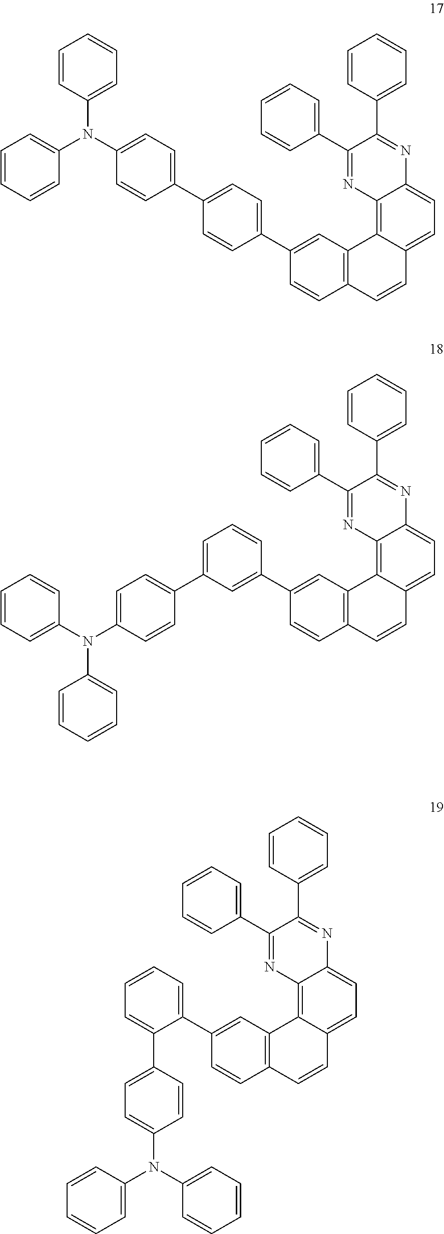 [SMILES: c1ccc(-c2nc3ccc4ccc5ccc(-c6ccc(-c7ccc(N(c8ccccc8)c8ccccc8)cc7)cc6)cc5c4c3nc2-c2ccccc2)cc1 ; c1ccc(-c2nc3ccc4ccc5ccc(-c6cccc(-c7ccc(N(c8ccccc8)c8ccccc8)cc7)c6)cc5c4c3nc2-c2ccccc2)cc1 ; c1ccc(-c2nc3ccc4ccc5ccc(-c6ccccc6-c6ccc(N(c7ccccc7)c7ccccc7)cc6)cc5c4c3nc2-c2ccccc2)cc1]

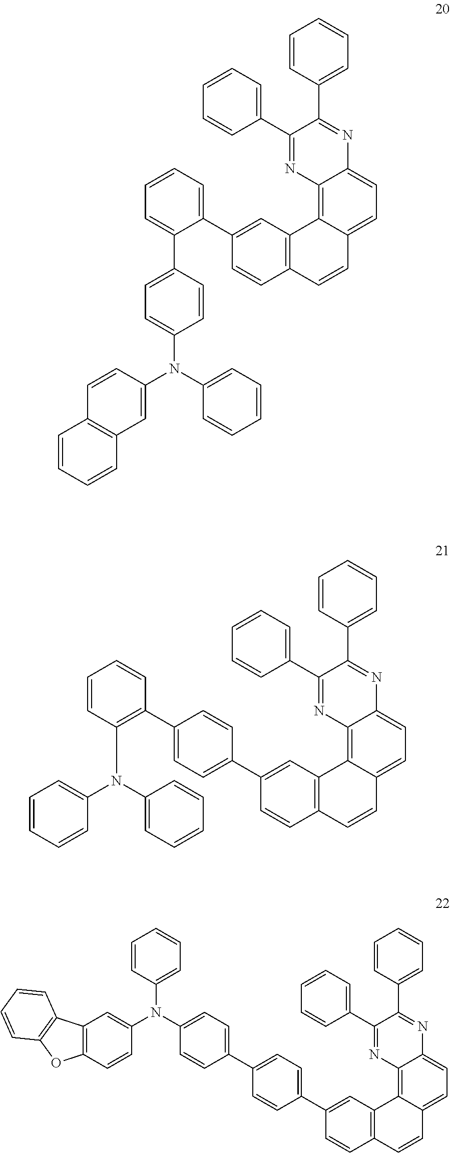 [SMILES: c1ccc(-c2nc3ccc4ccc5ccc(-c6ccccc6-c6ccc(N(c7ccccc7)c7ccc8ccccc8c7)cc6)cc5c4c3nc2-c2ccccc2)cc1 ; c1ccc(-c2nc3ccc4ccc5ccc(-c6ccc(-c7ccccc7N(c7ccccc7)c7ccccc7)cc6)cc5c4c3nc2-c2ccccc2)cc1 ; c1ccc(-c2nc3ccc4ccc5ccc(-c6ccc(-c7ccc(N(c8ccccc8)c8ccc9oc%10ccccc%10c9c8)cc7)cc6)cc5c4c3nc2-c2ccccc2)cc1]

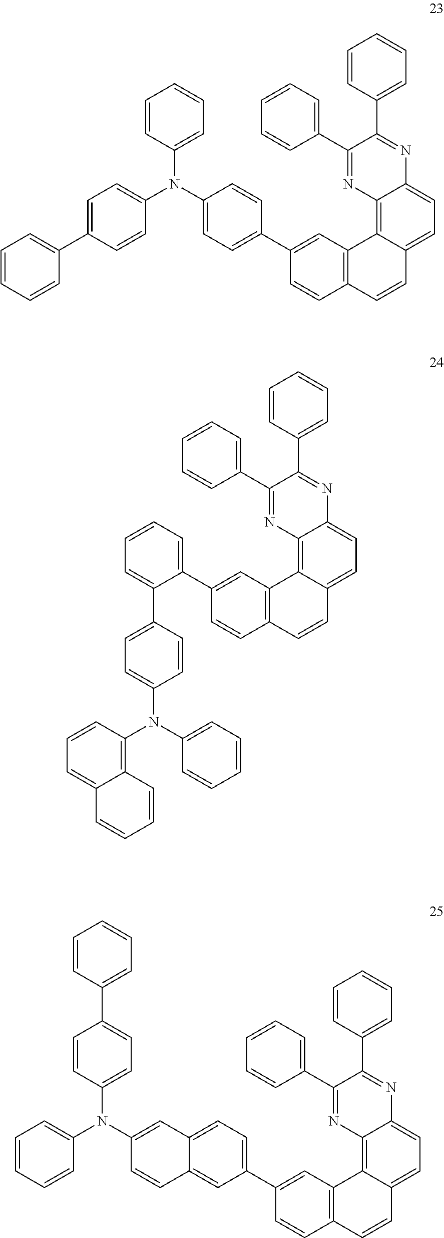 [SMILES: c1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; c1ccc(-c2nc3ccc4ccc5ccc(-c6ccccc6-c6ccc(N(c7ccccc7)c7cccc8ccccc78)cc6)cc5c4c3nc2-c2ccccc2)cc1 ; c1ccc(-c2ccc(N(c3ccccc3)c3ccc4cc(-c5ccc6ccc7ccc8nc(-c9ccccc9)c(-c9ccccc9)nc8c7c6c5)ccc4c3)cc2)cc1]

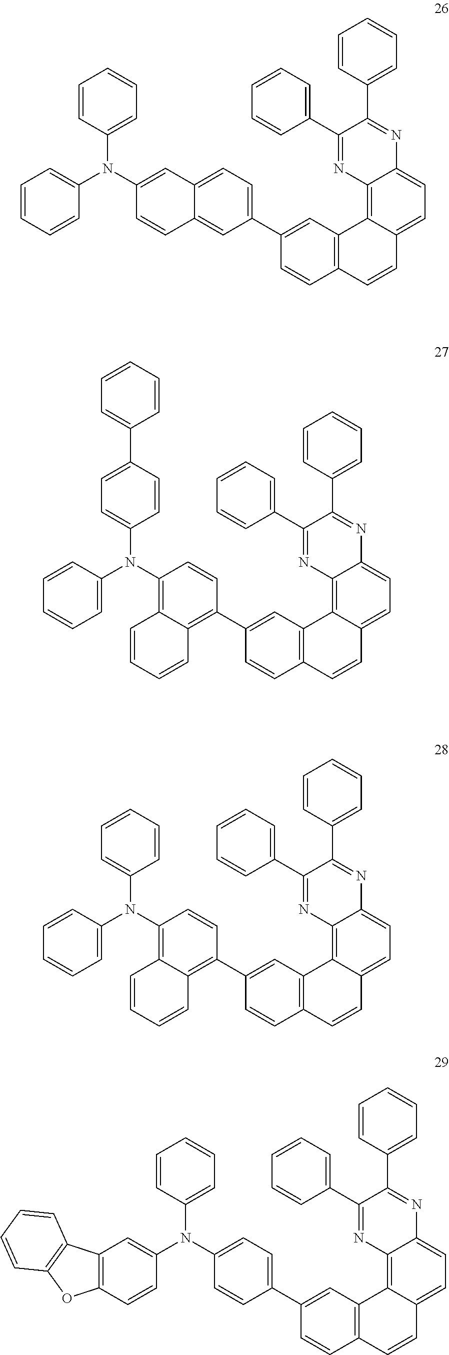 [SMILES: c1ccc(-c2nc3ccc4ccc5ccc(-c6ccc7cc(N(c8ccccc8)c8ccccc8)ccc7c6)cc5c4c3nc2-c2ccccc2)cc1 ; c1ccc(-c2ccc(N(c3ccccc3)c3ccc(-c4ccc5ccc6ccc7nc(-c8ccccc8)c(-c8ccccc8)nc7c6c5c4)c4ccccc34)cc2)cc1 ; c1ccc(-c2nc3ccc4ccc5ccc(-c6ccc(N(c7ccccc7)c7ccccc7)c7ccccc67)cc5c4c3nc2-c2ccccc2)cc1 ; c1ccc(-c2nc3ccc4ccc5ccc(-c6ccc(N(c7ccccc7)c7ccc8oc9ccccc9c8c7)cc6)cc5c4c3nc2-c2ccccc2)cc1]

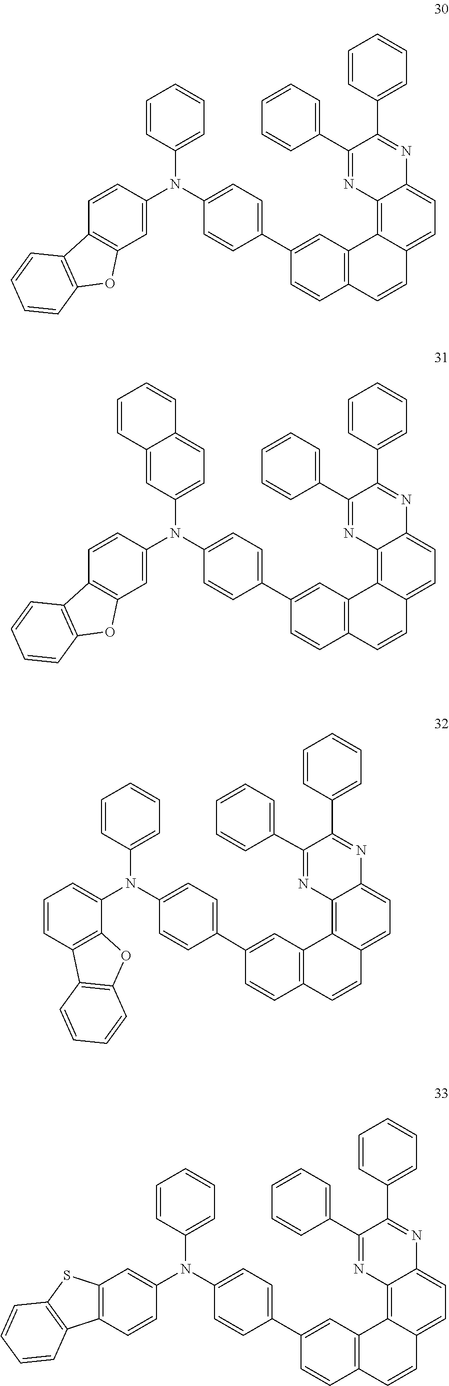 [SMILES: c1ccc(-c2nc3ccc4ccc5ccc(-c6ccc(N(c7ccccc7)c7ccc8c(c7)oc7ccccc78)cc6)cc5c4c3nc2-c2ccccc2)cc1 ; c1ccc(-c2nc3ccc4ccc5ccc(-c6ccc(N(c7ccc8ccccc8c7)c7ccc8c(c7)oc7ccccc78)cc6)cc5c4c3nc2-c2ccccc2)cc1 ; c1ccc(-c2nc3ccc4ccc5ccc(-c6ccc(N(c7ccccc7)c7cccc8c7oc7ccccc78)cc6)cc5c4c3nc2-c2ccccc2)cc1 ; c1ccc(-c2nc3ccc4ccc5ccc(-c6ccc(N(c7ccccc7)c7ccc8c(c7)sc7ccccc78)cc6)cc5c4c3nc2-c2ccccc2)cc1]

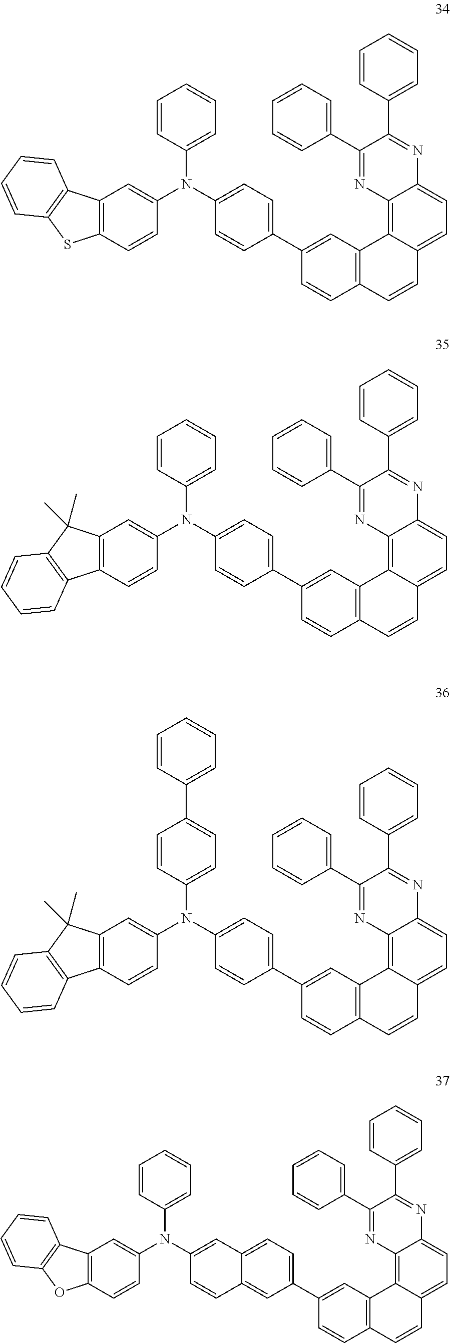 [SMILES: c1ccc(-c2nc3ccc4ccc5ccc(-c6ccc(N(c7ccccc7)c7ccc8sc9ccccc9c8c7)cc6)cc5c4c3nc2-c2ccccc2)cc1 ; CC1(C)c2ccccc2-c2ccc(N(c3ccccc3)c3ccc(-c4ccc5ccc6ccc7nc(-c8ccccc8)c(-c8ccccc8)nc7c6c5c4)cc3)cc21 ; CC1(C)c2ccccc2-c2ccc(N(c3ccc(-c4ccccc4)cc3)c3ccc(-c4ccc5ccc6ccc7nc(-c8ccccc8)c(-c8ccccc8)nc7c6c5c4)cc3)cc21 ; c1ccc(-c2nc3ccc4ccc5ccc(-c6ccc7cc(N(c8ccccc8)c8ccc9oc%10ccccc%10c9c8)ccc7c6)cc5c4c3nc2-c2ccccc2)cc1]

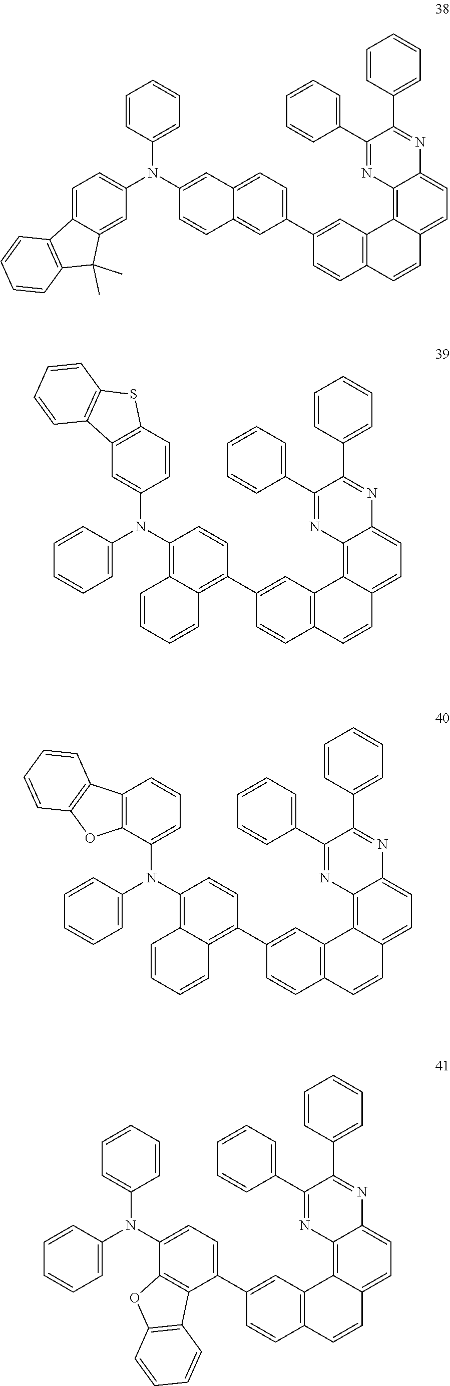 [SMILES: CC1(C)c2ccccc2-c2ccc(N(c3ccccc3)c3ccc4cc(-c5ccc6ccc7ccc8nc(-c9ccccc9)c(-c9ccccc9)nc8c7c6c5)ccc4c3)cc21 ; c1ccc(-c2nc3ccc4ccc5ccc(-c6ccc(N(c7ccccc7)c7ccc8sc9ccccc9c8c7)c7ccccc67)cc5c4c3nc2-c2ccccc2)cc1 ; c1ccc(-c2nc3ccc4ccc5ccc(-c6ccc(N(c7ccccc7)c7cccc8c7oc7ccccc78)c7ccccc67)cc5c4c3nc2-c2ccccc2)cc1 ; c1ccc(-c2nc3ccc4ccc5ccc(-c6ccc(N(c7ccccc7)c7ccccc7)c7oc8ccccc8c67)cc5c4c3nc2-c2ccccc2)cc1]

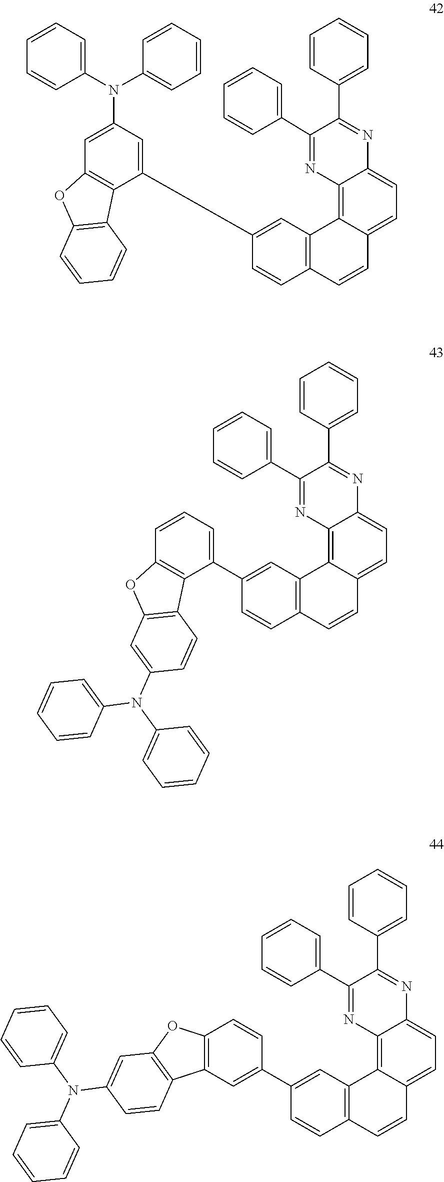 [SMILES: c1ccc(-c2nc3ccc4ccc5ccc(-c6cc(N(c7ccccc7)c7ccccc7)cc7oc8ccccc8c67)cc5c4c3nc2-c2ccccc2)cc1 ; c1ccc(-c2nc3ccc4ccc5ccc(-c6cccc7oc8cc(N(c9ccccc9)c9ccccc9)ccc8c67)cc5c4c3nc2-c2ccccc2)cc1 ; c1ccc(-c2nc3ccc4ccc5ccc(-c6ccc7oc8cc(N(c9ccccc9)c9ccccc9)ccc8c7c6)cc5c4c3nc2-c2ccccc2)cc1]

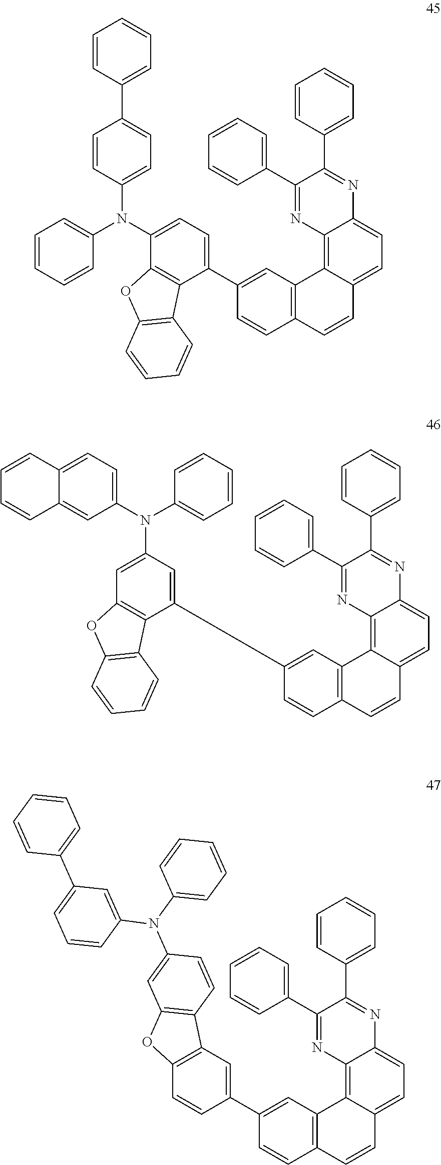 [SMILES: c1ccc(-c2ccc(N(c3ccccc3)c3ccc(-c4ccc5ccc6ccc7nc(-c8ccccc8)c(-c8ccccc8)nc7c6c5c4)c4c3oc3ccccc34)cc2)cc1 ; c1ccc(-c2nc3ccc4ccc5ccc(-c6cc(N(c7ccccc7)c7ccc8ccccc8c7)cc7oc8ccccc8c67)cc5c4c3nc2-c2ccccc2)cc1 ; c1ccc(-c2cccc(N(c3ccccc3)c3ccc4c(c3)oc3ccc(-c5ccc6ccc7ccc8nc(-c9ccccc9)c(-c9ccccc9)nc8c7c6c5)cc34)c2)cc1]

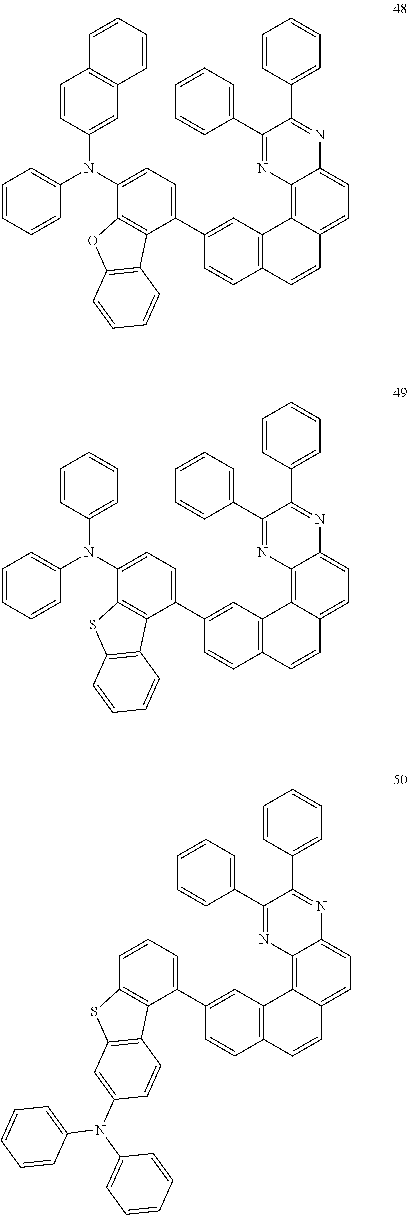 [SMILES: c1ccc(-c2nc3ccc4ccc5ccc(-c6ccc(N(c7ccccc7)c7ccc8ccccc8c7)c7oc8ccccc8c67)cc5c4c3nc2-c2ccccc2)cc1 ; c1ccc(-c2nc3ccc4ccc5ccc(-c6ccc(N(c7ccccc7)c7ccccc7)c7sc8ccccc8c67)cc5c4c3nc2-c2ccccc2)cc1 ; c1ccc(-c2nc3ccc4ccc5ccc(-c6cccc7sc8cc(N(c9ccccc9)c9ccccc9)ccc8c67)cc5c4c3nc2-c2ccccc2)cc1]

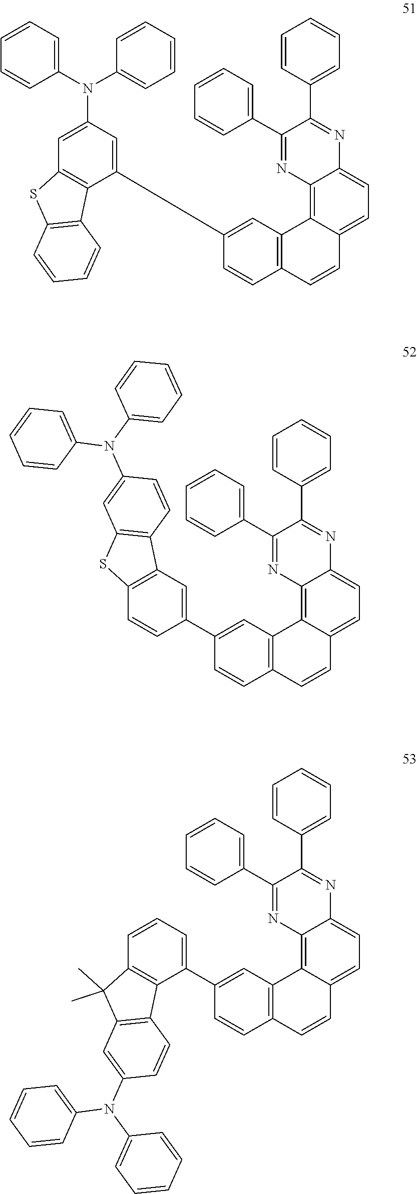 [SMILES: c1ccc(-c2nc3ccc4ccc5ccc(-c6cc(N(c7ccccc7)c7ccccc7)cc7sc8ccccc8c67)cc5c4c3nc2-c2ccccc2)cc1 ; c1ccc(-c2nc3ccc4ccc5ccc(-c6ccc7sc8cc(N(c9ccccc9)c9ccccc9)ccc8c7c6)cc5c4c3nc2-c2ccccc2)cc1 ; CC1(C)c2cc(N(c3ccccc3)c3ccccc3)ccc2-c2c(-c3ccc4ccc5ccc6nc(-c7ccccc7)c(-c7ccccc7)nc6c5c4c3)cccc21]

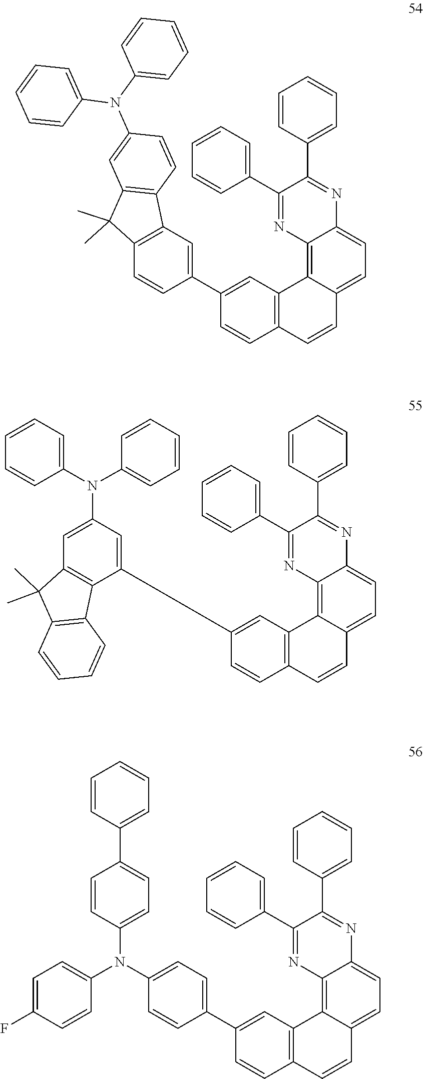 [SMILES: CC1(C)c2ccc(-c3ccc4ccc5ccc6nc(-c7ccccc7)c(-c7ccccc7)nc6c5c4c3)cc2-c2ccc(N(c3ccccc3)c3ccccc3)cc21 ; CC1(C)c2ccccc2-c2c(-c3ccc4ccc5ccc6nc(-c7ccccc7)c(-c7ccccc7)nc6c5c4c3)cc(N(c3ccccc3)c3ccccc3)cc21 ; Fc1ccc(N(c2ccc(-c3ccccc3)cc2)c2ccc(-c3ccc4ccc5ccc6nc(-c7ccccc7)c(-c7ccccc7)nc6c5c4c3)cc2)cc1]

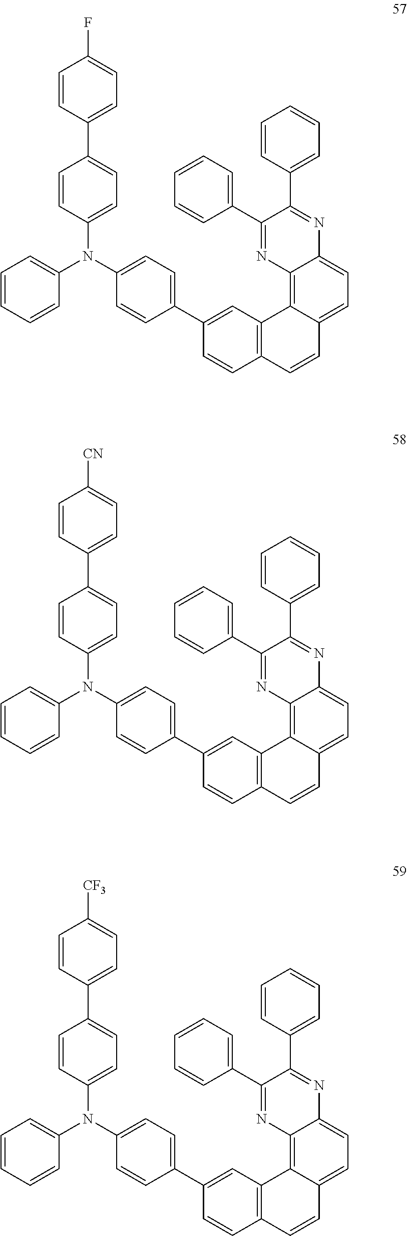 [SMILES: Fc1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; N#Cc1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; FC(F)(F)c1ccc(-c2ccc(N(c3ccccc3)c3ccc(-c4ccc5ccc6ccc7nc(-c8ccccc8)c(-c8ccccc8)nc7c6c5c4)cc3)cc2)cc1]

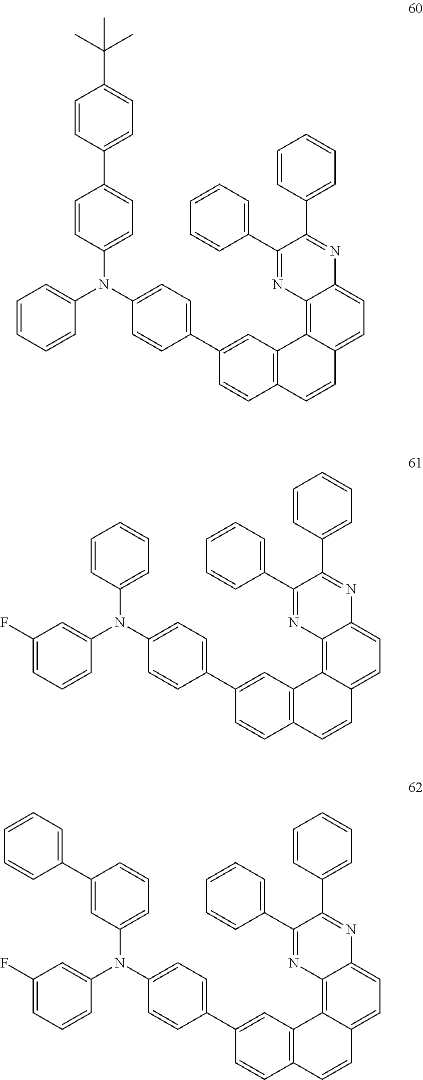 [SMILES: CC(C)(C)c1ccc(-c2ccc(N(c3ccccc3)c3ccc(-c4ccc5ccc6ccc7nc(-c8ccccc8)c(-c8ccccc8)nc7c6c5c4)cc3)cc2)cc1 ; Fc1cccc(N(c2ccccc2)c2ccc(-c3ccc4ccc5ccc6nc(-c7ccccc7)c(-c7ccccc7)nc6c5c4c3)cc2)c1 ; Fc1cccc(N(c2ccc(-c3ccc4ccc5ccc6nc(-c7ccccc7)c(-c7ccccc7)nc6c5c4c3)cc2)c2cccc(-c3ccccc3)c2)c1]

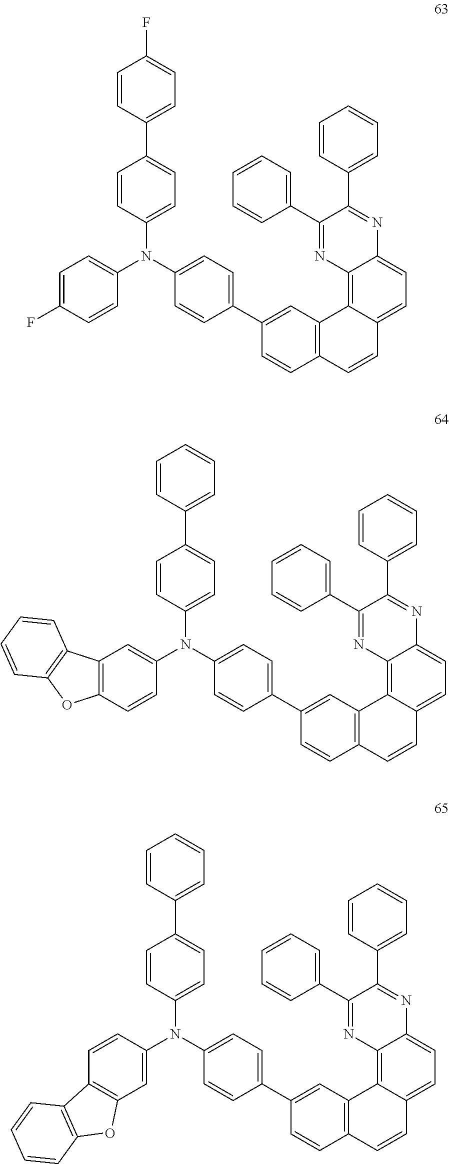 [SMILES: Fc1ccc(-c2ccc(N(c3ccc(F)cc3)c3ccc(-c4ccc5ccc6ccc7nc(-c8ccccc8)c(-c8ccccc8)nc7c6c5c4)cc3)cc2)cc1 ; c1ccc(-c2ccc(N(c3ccc(-c4ccc5ccc6ccc7nc(-c8ccccc8)c(-c8ccccc8)nc7c6c5c4)cc3)c3ccc4oc5ccccc5c4c3)cc2)cc1 ; c1ccc(-c2ccc(N(c3ccc(-c4ccc5ccc6ccc7nc(-c8ccccc8)c(-c8ccccc8)nc7c6c5c4)cc3)c3ccc4c(c3)oc3ccccc34)cc2)cc1]

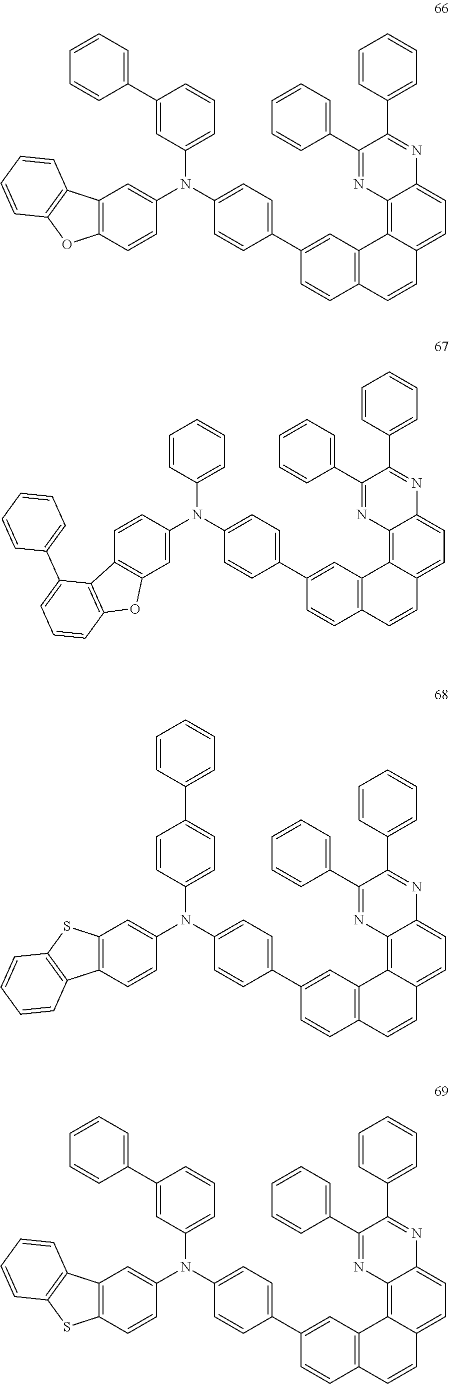 [SMILES: c1ccc(-c2cccc(N(c3ccc(-c4ccc5ccc6ccc7nc(-c8ccccc8)c(-c8ccccc8)nc7c6c5c4)cc3)c3ccc4oc5ccccc5c4c3)c2)cc1 ; c1ccc(-c2nc3ccc4ccc5ccc(-c6ccc(N(c7ccccc7)c7ccc8c(c7)oc7cccc(-c9ccccc9)c78)cc6)cc5c4c3nc2-c2ccccc2)cc1 ; c1ccc(-c2ccc(N(c3ccc(-c4ccc5ccc6ccc7nc(-c8ccccc8)c(-c8ccccc8)nc7c6c5c4)cc3)c3ccc4c(c3)sc3ccccc34)cc2)cc1 ; c1ccc(-c2cccc(N(c3ccc(-c4ccc5ccc6ccc7nc(-c8ccccc8)c(-c8ccccc8)nc7c6c5c4)cc3)c3ccc4sc5ccccc5c4c3)c2)cc1]

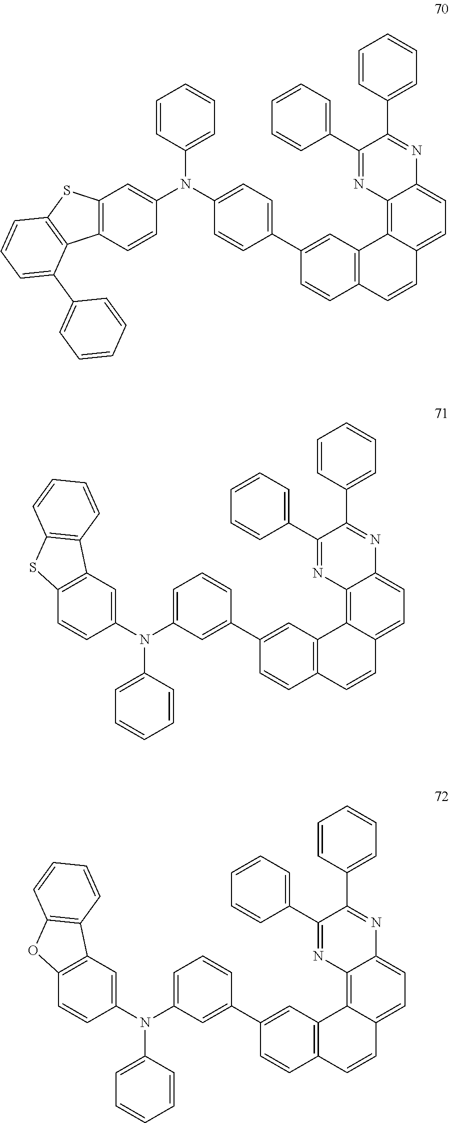 [SMILES: c1ccc(-c2nc3ccc4ccc5ccc(-c6ccc(N(c7ccccc7)c7ccc8c(c7)sc7cccc(-c9ccccc9)c78)cc6)cc5c4c3nc2-c2ccccc2)cc1 ; c1ccc(-c2nc3ccc4ccc5ccc(-c6cccc(N(c7ccccc7)c7ccc8sc9ccccc9c8c7)c6)cc5c4c3nc2-c2ccccc2)cc1 ; c1ccc(-c2nc3ccc4ccc5ccc(-c6cccc(N(c7ccccc7)c7ccc8oc9ccccc9c8c7)c6)cc5c4c3nc2-c2ccccc2)cc1]

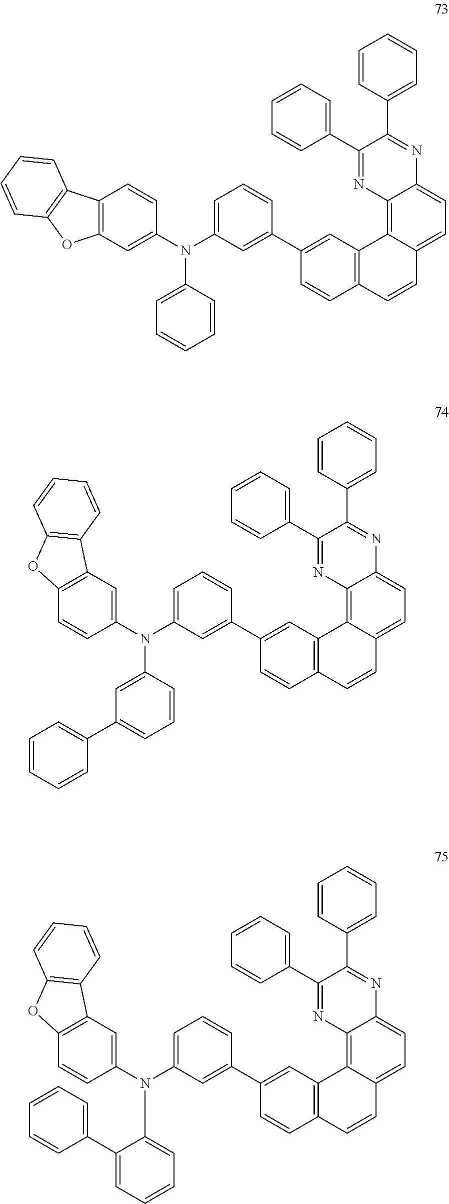 [SMILES: c1ccc(-c2nc3ccc4ccc5ccc(-c6cccc(N(c7ccccc7)c7ccc8c(c7)oc7ccccc78)c6)cc5c4c3nc2-c2ccccc2)cc1 ; c1ccc(-c2cccc(N(c3cccc(-c4ccc5ccc6ccc7nc(-c8ccccc8)c(-c8ccccc8)nc7c6c5c4)c3)c3ccc4oc5ccccc5c4c3)c2)cc1 ; c1ccc(-c2ccccc2N(c2cccc(-c3ccc4ccc5ccc6nc(-c7ccccc7)c(-c7ccccc7)nc6c5c4c3)c2)c2ccc3oc4ccccc4c3c2)cc1]

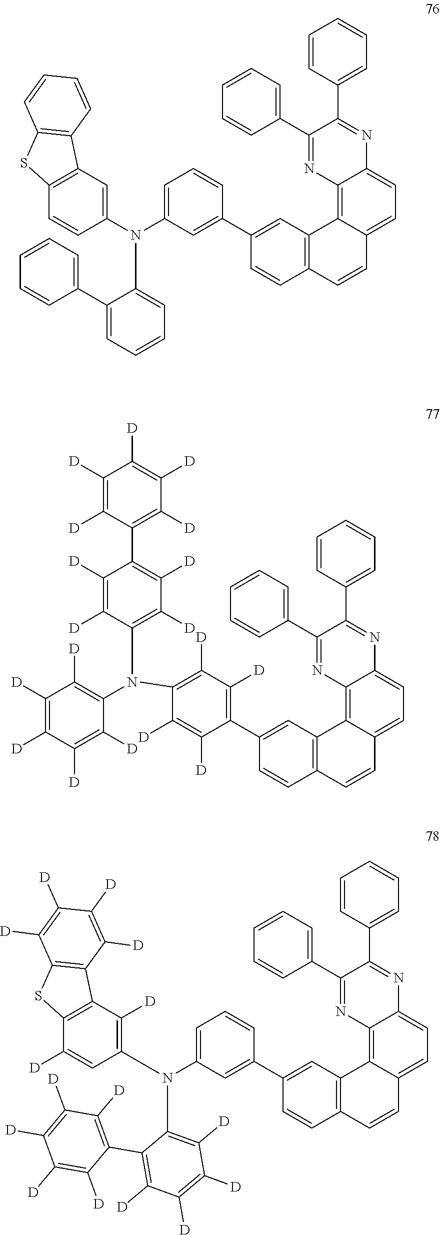 [SMILES: c1ccc(-c2ccccc2N(c2cccc(-c3ccc4ccc5ccc6nc(-c7ccccc7)c(-c7ccccc7)nc6c5c4c3)c2)c2ccc3sc4ccccc4c3c2)cc1 ; [2H]c1c([2H])c([2H])c(-c2c([2H])c([2H])c(N(c3c([2H])c([2H])c([2H])c([2H])c3[2H])c3c([2H])c([2H])c(-c4ccc5ccc6ccc7nc(-c8ccccc8)c(-c8ccccc8)nc7c6c5c4)c([2H])c3[2H])c([2H])c2[2H])c([2H])c1[2H] ; [2H]c1c([2H])c([2H])c(-c2c([2H])c([2H])c([2H])c([2H])c2N(c2cccc(-c3ccc4ccc5ccc6nc(-c7ccccc7)c(-c7ccccc7)nc6c5c4c3)c2)c2cc([2H])c3sc4c([2H])c([2H])c([2H])c([2H])c4c3c2[2H])c([2H])c1[2H]]

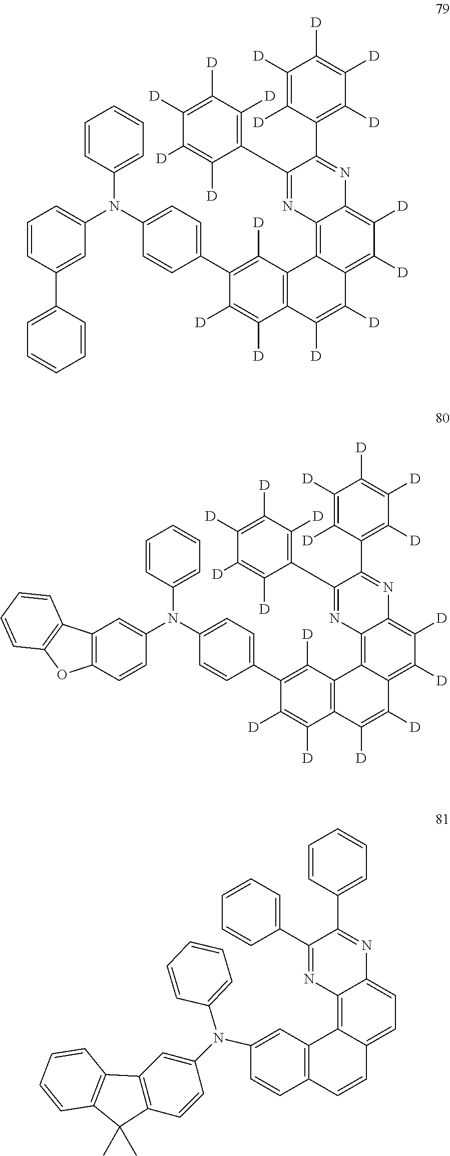 [SMILES: [2H]c1c([2H])c([2H])c(-c2nc3c([2H])c([2H])c4c([2H])c([2H])c5c([2H])c([2H])c(-c6ccc(N(c7ccccc7)c7cccc(-c8ccccc8)c7)cc6)c([2H])c5c4c3nc2-c2c([2H])c([2H])c([2H])c([2H])c2[2H])c([2H])c1[2H] ; [2H]c1c([2H])c([2H])c(-c2nc3c([2H])c([2H])c4c([2H])c([2H])c5c([2H])c([2H])c(-c6ccc(N(c7ccccc7)c7ccc8oc9ccccc9c8c7)cc6)c([2H])c5c4c3nc2-c2c([2H])c([2H])c([2H])c([2H])c2[2H])c([2H])c1[2H] ; CC1(C)c2ccccc2-c2cc(N(c3ccccc3)c3ccc4ccc5ccc6nc(-c7ccccc7)c(-c7ccccc7)nc6c5c4c3)ccc21]

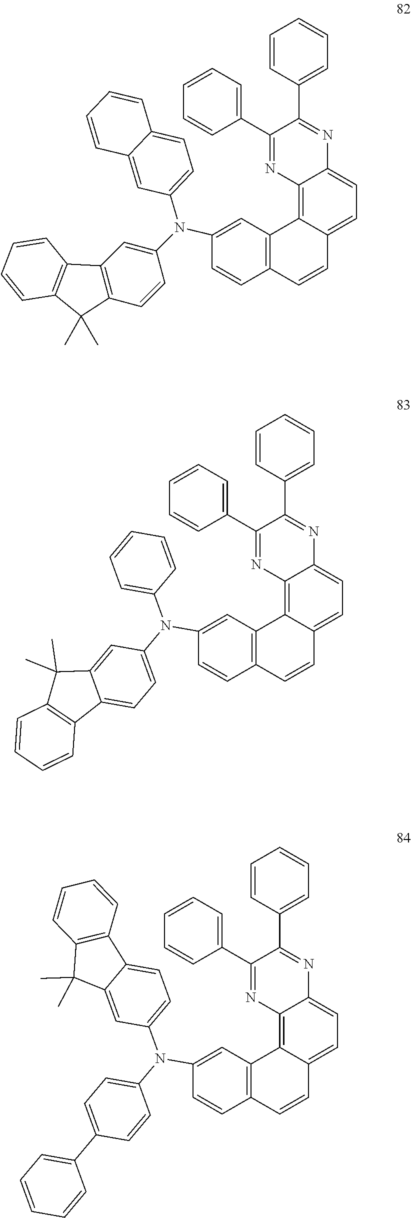 [SMILES: CC1(C)c2ccccc2-c2cc(N(c3ccc4ccccc4c3)c3ccc4ccc5ccc6nc(-c7ccccc7)c(-c7ccccc7)nc6c5c4c3)ccc21 ; CC1(C)c2ccccc2-c2ccc(N(c3ccccc3)c3ccc4ccc5ccc6nc(-c7ccccc7)c(-c7ccccc7)nc6c5c4c3)cc21 ; CC1(C)c2ccccc2-c2ccc(N(c3ccc(-c4ccccc4)cc3)c3ccc4ccc5ccc6nc(-c7ccccc7)c(-c7ccccc7)nc6c5c4c3)cc21]

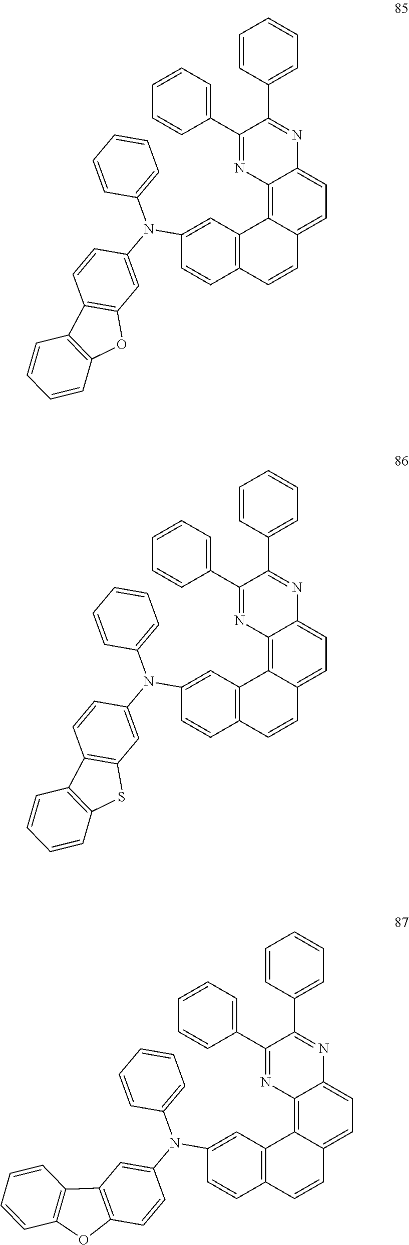 [SMILES: c1ccc(-c2nc3ccc4ccc5ccc(N(c6ccccc6)c6ccc7c(c6)oc6ccccc67)cc5c4c3nc2-c2ccccc2)cc1 ; c1ccc(-c2nc3ccc4ccc5ccc(N(c6ccccc6)c6ccc7c(c6)sc6ccccc67)cc5c4c3nc2-c2ccccc2)cc1 ; c1ccc(-c2nc3ccc4ccc5ccc(N(c6ccccc6)c6ccc7oc8ccccc8c7c6)cc5c4c3nc2-c2ccccc2)cc1]

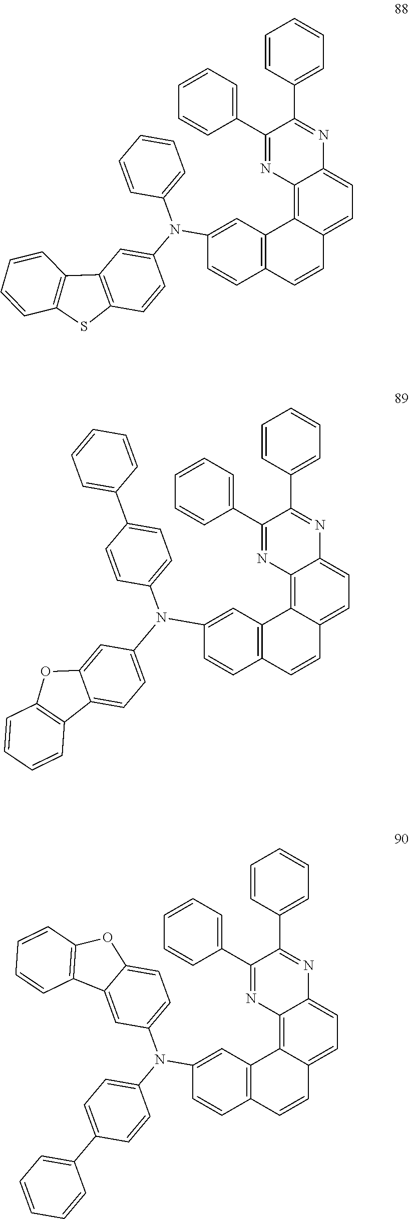 [SMILES: c1ccc(-c2nc3ccc4ccc5ccc(N(c6ccccc6)c6ccc7sc8ccccc8c7c6)cc5c4c3nc2-c2ccccc2)cc1 ; c1ccc(-c2ccc(N(c3ccc4c(c3)oc3ccccc34)c3ccc4ccc5ccc6nc(-c7ccccc7)c(-c7ccccc7)nc6c5c4c3)cc2)cc1 ; c1ccc(-c2ccc(N(c3ccc4oc5ccccc5c4c3)c3ccc4ccc5ccc6nc(-c7ccccc7)c(-c7ccccc7)nc6c5c4c3)cc2)cc1]

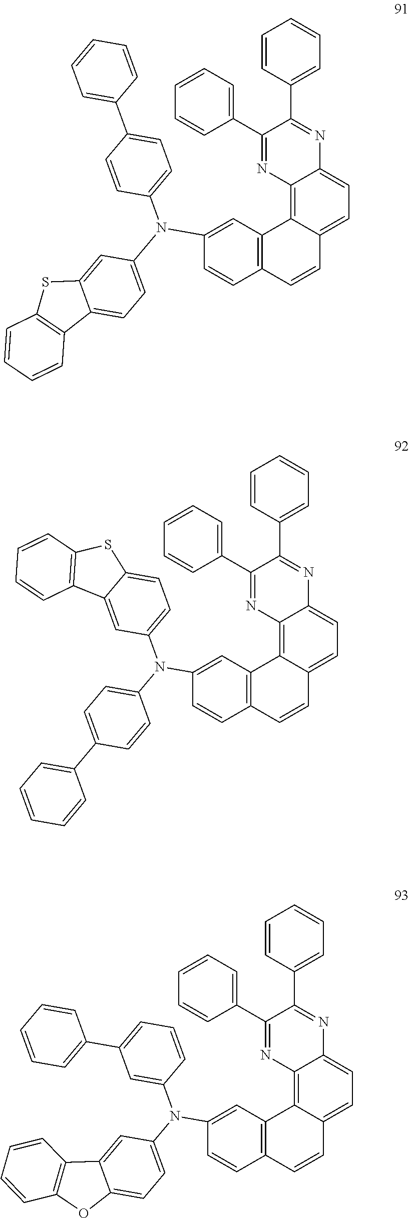 [SMILES: c1ccc(-c2ccc(N(c3ccc4c(c3)sc3ccccc34)c3ccc4ccc5ccc6nc(-c7ccccc7)c(-c7ccccc7)nc6c5c4c3)cc2)cc1 ; c1ccc(-c2ccc(N(c3ccc4sc5ccccc5c4c3)c3ccc4ccc5ccc6nc(-c7ccccc7)c(-c7ccccc7)nc6c5c4c3)cc2)cc1 ; c1ccc(-c2cccc(N(c3ccc4oc5ccccc5c4c3)c3ccc4ccc5ccc6nc(-c7ccccc7)c(-c7ccccc7)nc6c5c4c3)c2)cc1]

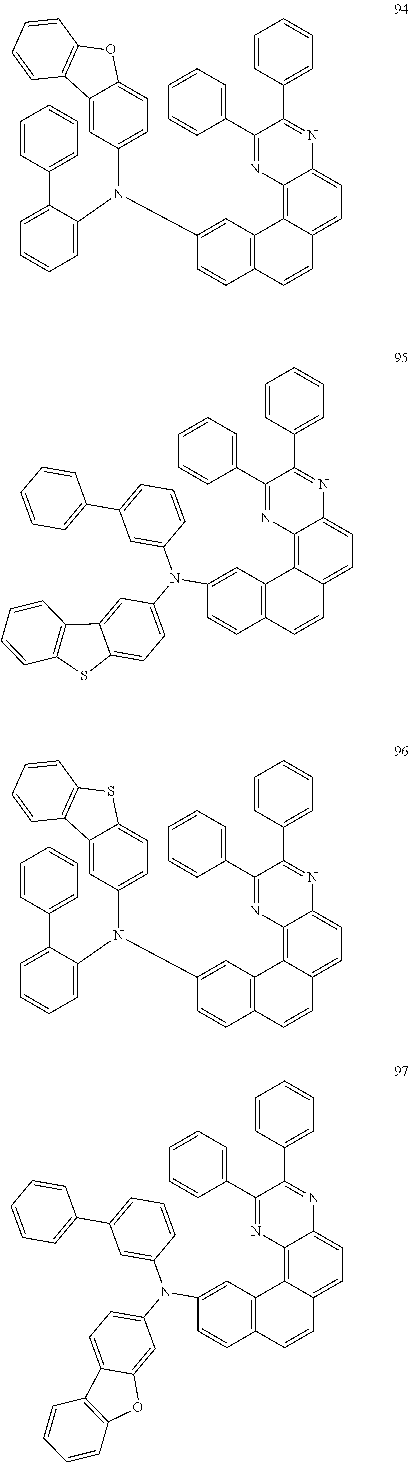 [SMILES: c1ccc(-c2ccccc2N(c2ccc3oc4ccccc4c3c2)c2ccc3ccc4ccc5nc(-c6ccccc6)c(-c6ccccc6)nc5c4c3c2)cc1 ; c1ccc(-c2cccc(N(c3ccc4sc5ccccc5c4c3)c3ccc4ccc5ccc6nc(-c7ccccc7)c(-c7ccccc7)nc6c5c4c3)c2)cc1 ; c1ccc(-c2ccccc2N(c2ccc3sc4ccccc4c3c2)c2ccc3ccc4ccc5nc(-c6ccccc6)c(-c6ccccc6)nc5c4c3c2)cc1 ; c1ccc(-c2cccc(N(c3ccc4c(c3)oc3ccccc34)c3ccc4ccc5ccc6nc(-c7ccccc7)c(-c7ccccc7)nc6c5c4c3)c2)cc1]

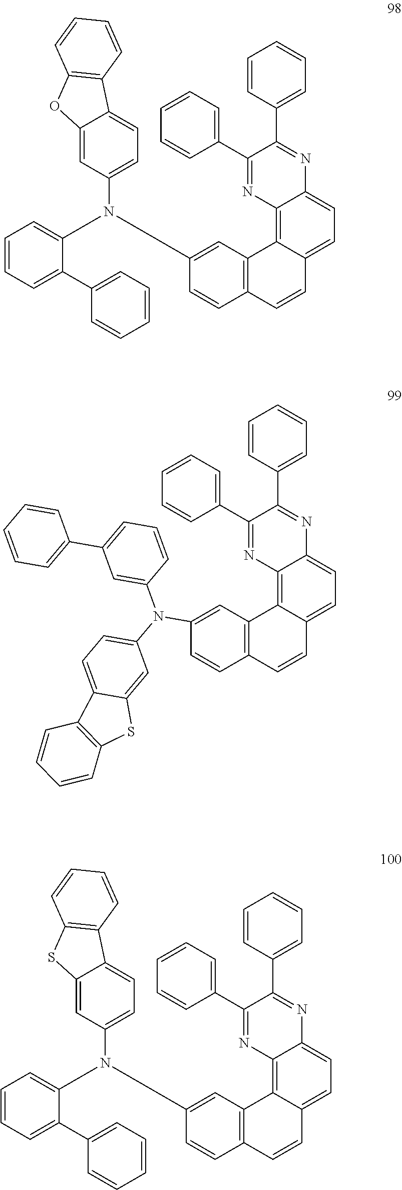 [SMILES: c1ccc(-c2ccccc2N(c2ccc3c(c2)oc2ccccc23)c2ccc3ccc4ccc5nc(-c6ccccc6)c(-c6ccccc6)nc5c4c3c2)cc1 ; c1ccc(-c2cccc(N(c3ccc4c(c3)sc3ccccc34)c3ccc4ccc5ccc6nc(-c7ccccc7)c(-c7ccccc7)nc6c5c4c3)c2)cc1 ; c1ccc(-c2ccccc2N(c2ccc3c(c2)sc2ccccc23)c2ccc3ccc4ccc5nc(-c6ccccc6)c(-c6ccccc6)nc5c4c3c2)cc1]

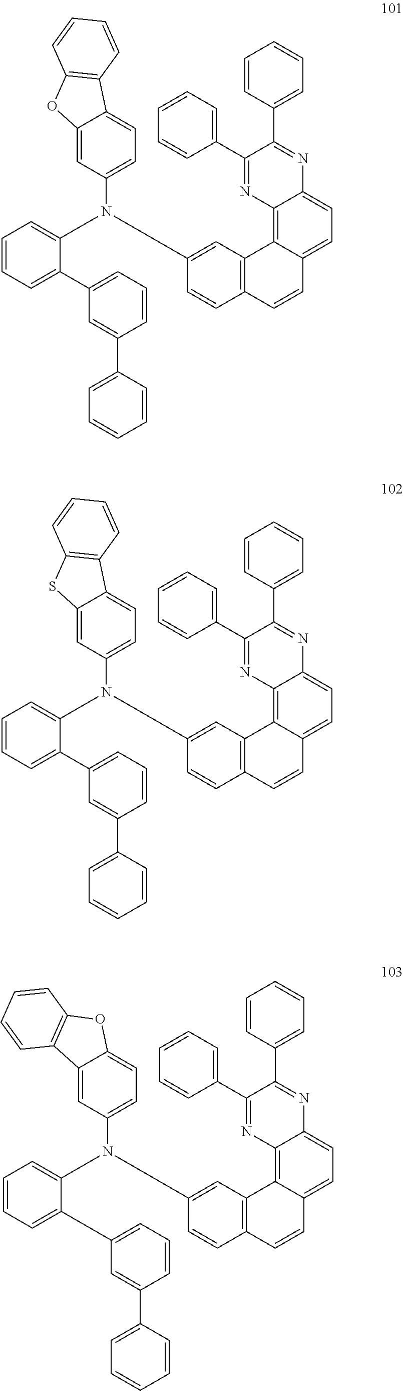 [SMILES: c1ccc(-c2cccc(-c3ccccc3N(c3ccc4c(c3)oc3ccccc34)c3ccc4ccc5ccc6nc(-c7ccccc7)c(-c7ccccc7)nc6c5c4c3)c2)cc1 ; c1ccc(-c2cccc(-c3ccccc3N(c3ccc4c(c3)sc3ccccc34)c3ccc4ccc5ccc6nc(-c7ccccc7)c(-c7ccccc7)nc6c5c4c3)c2)cc1 ; c1ccc(-c2cccc(-c3ccccc3N(c3ccc4oc5ccccc5c4c3)c3ccc4ccc5ccc6nc(-c7ccccc7)c(-c7ccccc7)nc6c5c4c3)c2)cc1]

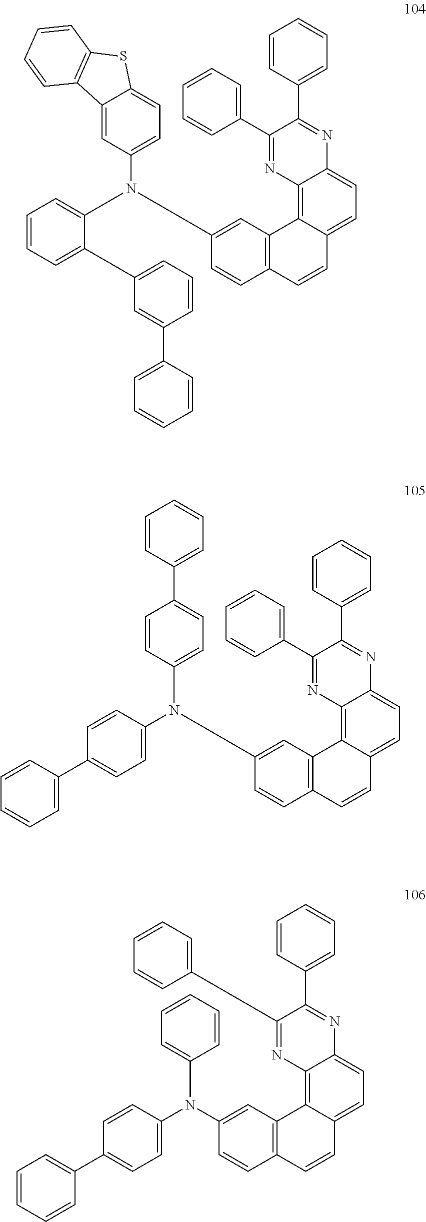 [SMILES: c1ccc(-c2cccc(-c3ccccc3N(c3ccc4sc5ccccc5c4c3)c3ccc4ccc5ccc6nc(-c7ccccc7)c(-c7ccccc7)nc6c5c4c3)c2)cc1 ; c1ccc(-c2ccc(N(c3ccc(-c4ccccc4)cc3)c3ccc4ccc5ccc6nc(-c7ccccc7)c(-c7ccccc7)nc6c5c4c3)cc2)cc1 ; c1ccc(-c2ccc(N(c3ccccc3)c3ccc4ccc5ccc6nc(-c7ccccc7)c(-c7ccccc7)nc6c5c4c3)cc2)cc1]

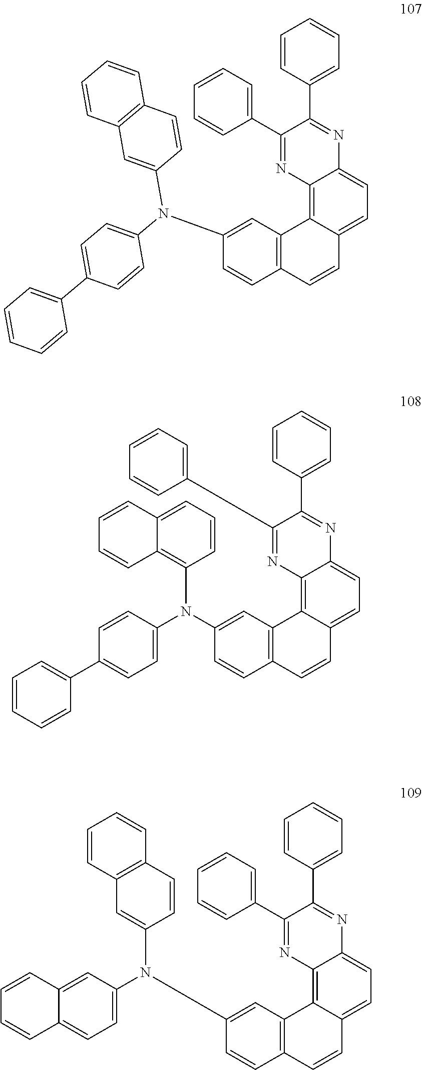 [SMILES: c1ccc(-c2ccc(N(c3ccc4ccccc4c3)c3ccc4ccc5ccc6nc(-c7ccccc7)c(-c7ccccc7)nc6c5c4c3)cc2)cc1 ; c1ccc(-c2ccc(N(c3ccc4ccc5ccc6nc(-c7ccccc7)c(-c7ccccc7)nc6c5c4c3)c3cccc4ccccc34)cc2)cc1 ; c1ccc(-c2nc3ccc4ccc5ccc(N(c6ccc7ccccc7c6)c6ccc7ccccc7c6)cc5c4c3nc2-c2ccccc2)cc1]

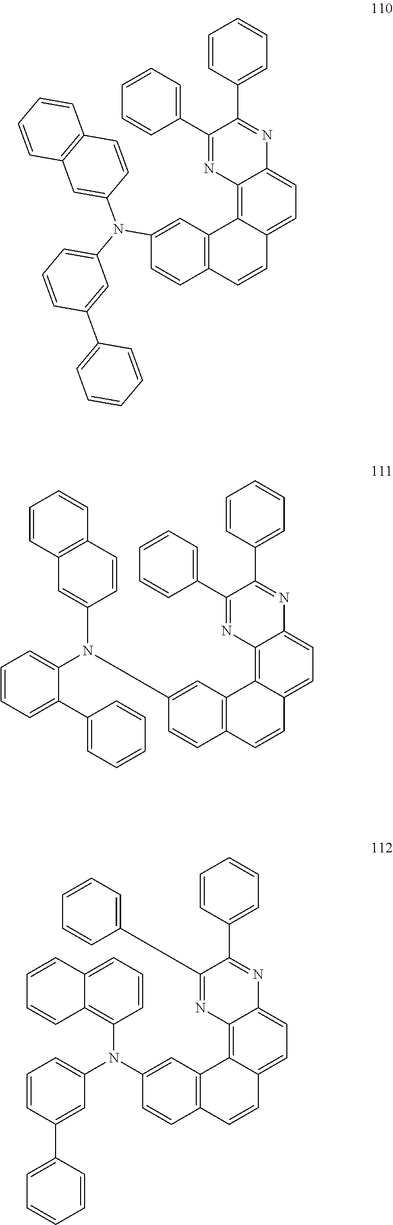 [SMILES: c1ccc(-c2cccc(N(c3ccc4ccccc4c3)c3ccc4ccc5ccc6nc(-c7ccccc7)c(-c7ccccc7)nc6c5c4c3)c2)cc1 ; c1ccc(-c2ccccc2N(c2ccc3ccccc3c2)c2ccc3ccc4ccc5nc(-c6ccccc6)c(-c6ccccc6)nc5c4c3c2)cc1 ; c1ccc(-c2cccc(N(c3ccc4ccc5ccc6nc(-c7ccccc7)c(-c7ccccc7)nc6c5c4c3)c3cccc4ccccc34)c2)cc1]

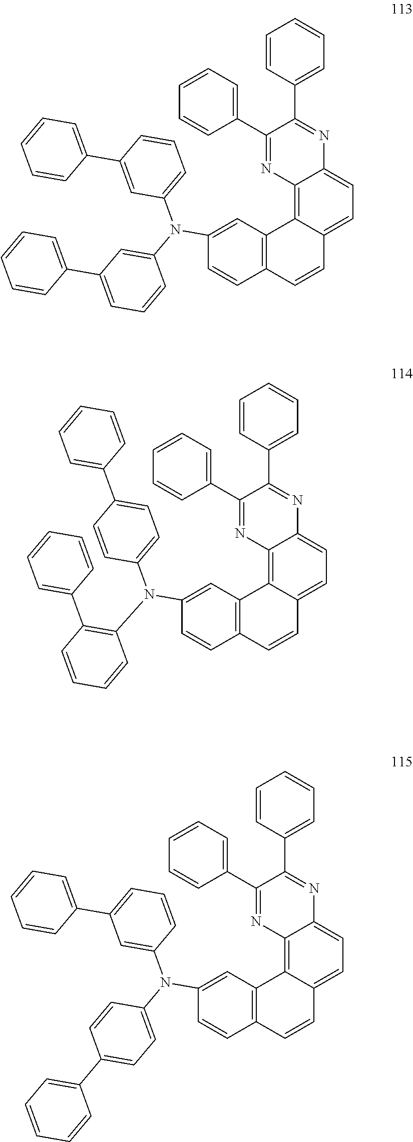 [SMILES: c1ccc(-c2cccc(N(c3cccc(-c4ccccc4)c3)c3ccc4ccc5ccc6nc(-c7ccccc7)c(-c7ccccc7)nc6c5c4c3)c2)cc1 ; c1ccc(-c2ccc(N(c3ccc4ccc5ccc6nc(-c7ccccc7)c(-c7ccccc7)nc6c5c4c3)c3ccccc3-c3ccccc3)cc2)cc1 ; c1ccc(-c2ccc(N(c3cccc(-c4ccccc4)c3)c3ccc4ccc5ccc6nc(-c7ccccc7)c(-c7ccccc7)nc6c5c4c3)cc2)cc1]

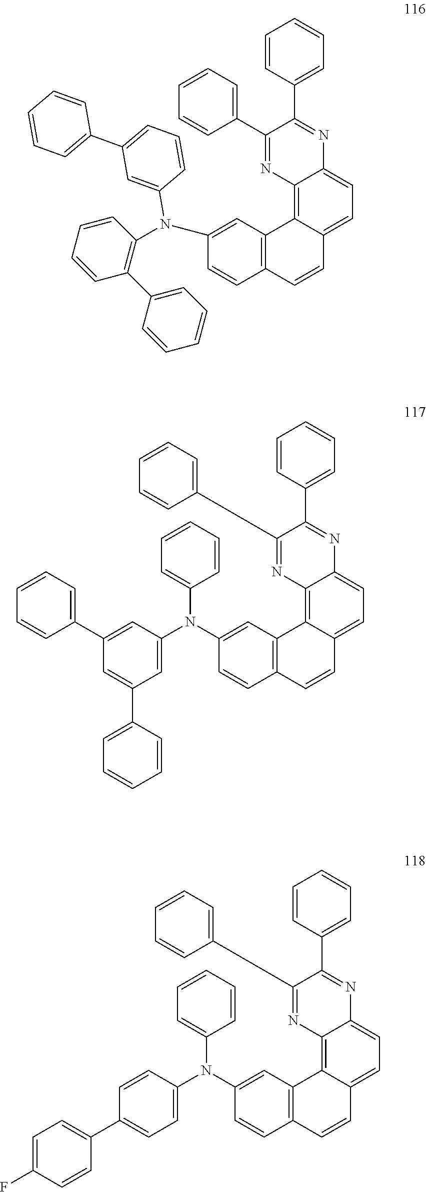 [SMILES: c1ccc(-c2cccc(N(c3ccc4ccc5ccc6nc(-c7ccccc7)c(-c7ccccc7)nc6c5c4c3)c3ccccc3-c3ccccc3)c2)cc1 ; c1ccc(-c2cc(-c3ccccc3)cc(N(c3ccccc3)c3ccc4ccc5ccc6nc(-c7ccccc7)c(-c7ccccc7)nc6c5c4c3)c2)cc1 ; Fc1ccc(-c2ccc(N(c3ccccc3)c3ccc4ccc5ccc6nc(-c7ccccc7)c(-c7ccccc7)nc6c5c4c3)cc2)cc1]

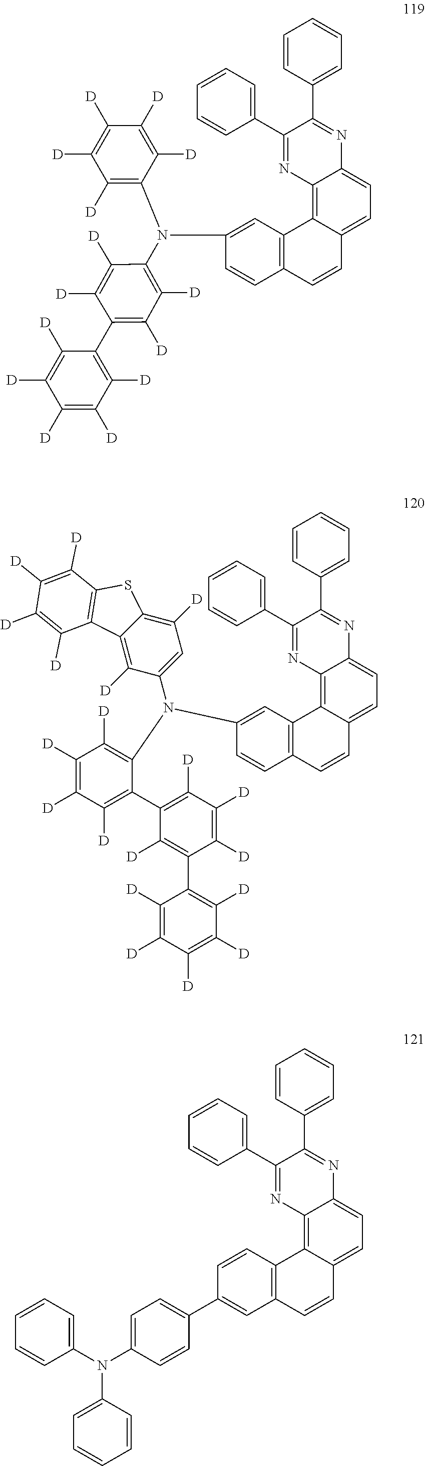 [SMILES: [2H]c1c([2H])c([2H])c(-c2c([2H])c([2H])c(N(c3ccc4ccc5ccc6nc(-c7ccccc7)c(-c7ccccc7)nc6c5c4c3)c3c([2H])c([2H])c([2H])c([2H])c3[2H])c([2H])c2[2H])c([2H])c1[2H] ; [2H]c1c([2H])c([2H])c(-c2c([2H])c([2H])c([2H])c(-c3c([2H])c([2H])c([2H])c([2H])c3N(c3ccc4ccc5ccc6nc(-c7ccccc7)c(-c7ccccc7)nc6c5c4c3)c3cc([2H])c4sc5c([2H])c([2H])c([2H])c([2H])c5c4c3[2H])c2[2H])c([2H])c1[2H] ; c1ccc(-c2nc3ccc4ccc5cc(-c6ccc(N(c7ccccc7)c7ccccc7)cc6)ccc5c4c3nc2-c2ccccc2)cc1]

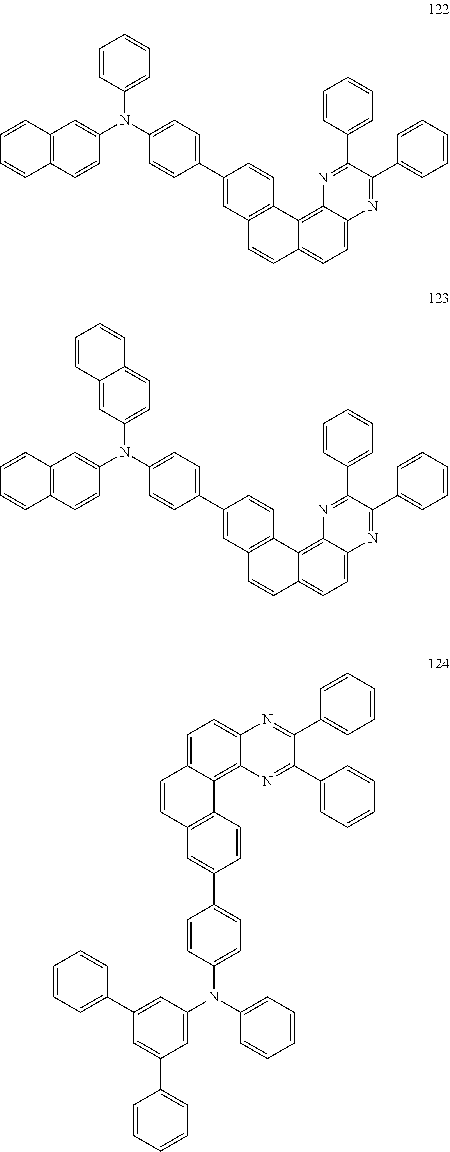 [SMILES: c1ccc(-c2nc3ccc4ccc5cc(-c6ccc(N(c7ccccc7)c7ccc8ccccc8c7)cc6)ccc5c4c3nc2-c2ccccc2)cc1 ; c1ccc(-c2nc3ccc4ccc5cc(-c6ccc(N(c7ccc8ccccc8c7)c7ccc8ccccc8c7)cc6)ccc5c4c3nc2-c2ccccc2)cc1 ; c1ccc(-c2cc(-c3ccccc3)cc(N(c3ccccc3)c3ccc(-c4ccc5c(ccc6ccc7nc(-c8ccccc8)c(-c8ccccc8)nc7c65)c4)cc3)c2)cc1]

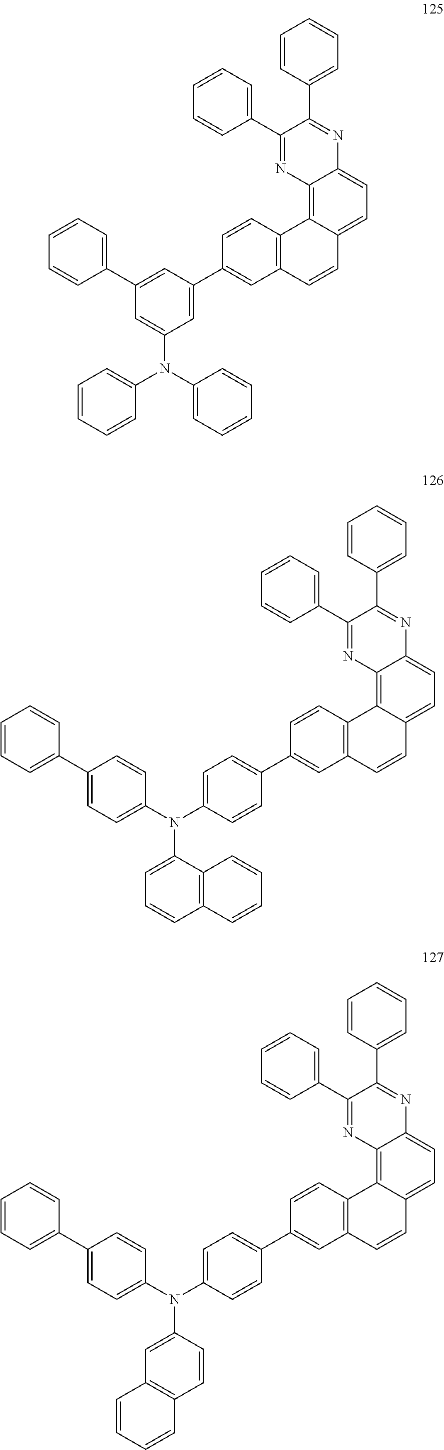 [SMILES: c1ccc(-c2cc(-c3ccc4c(ccc5ccc6nc(-c7ccccc7)c(-c7ccccc7)nc6c54)c3)cc(N(c3ccccc3)c3ccccc3)c2)cc1 ; c1ccc(-c2ccc(N(c3ccc(-c4ccc5c(ccc6ccc7nc(-c8ccccc8)c(-c8ccccc8)nc7c65)c4)cc3)c3cccc4ccccc34)cc2)cc1 ; c1ccc(-c2ccc(N(c3ccc(-c4ccc5c(ccc6ccc7nc(-c8ccccc8)c(-c8ccccc8)nc7c65)c4)cc3)c3ccc4ccccc4c3)cc2)cc1]

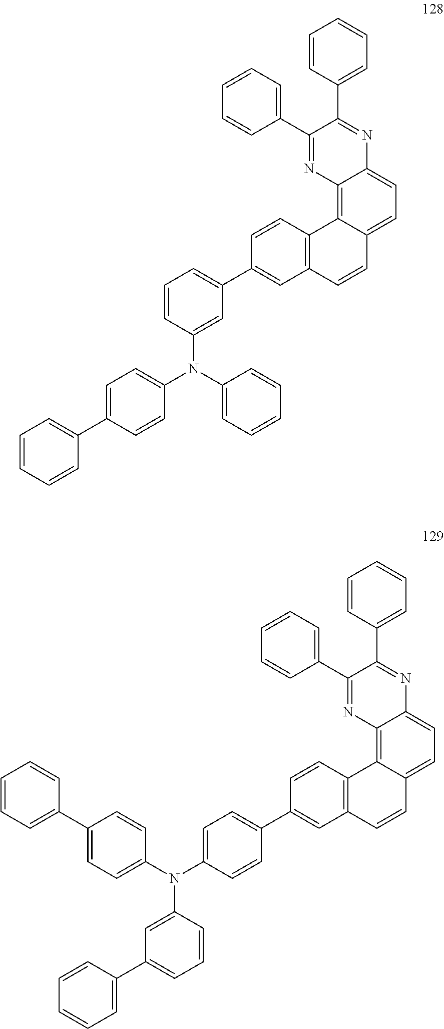 [SMILES: c1ccc(-c2ccc(N(c3ccccc3)c3cccc(-c4ccc5c(ccc6ccc7nc(-c8ccccc8)c(-c8ccccc8)nc7c65)c4)c3)cc2)cc1 ; c1ccc(-c2ccc(N(c3ccc(-c4ccc5c(ccc6ccc7nc(-c8ccccc8)c(-c8ccccc8)nc7c65)c4)cc3)c3cccc(-c4ccccc4)c3)cc2)cc1]

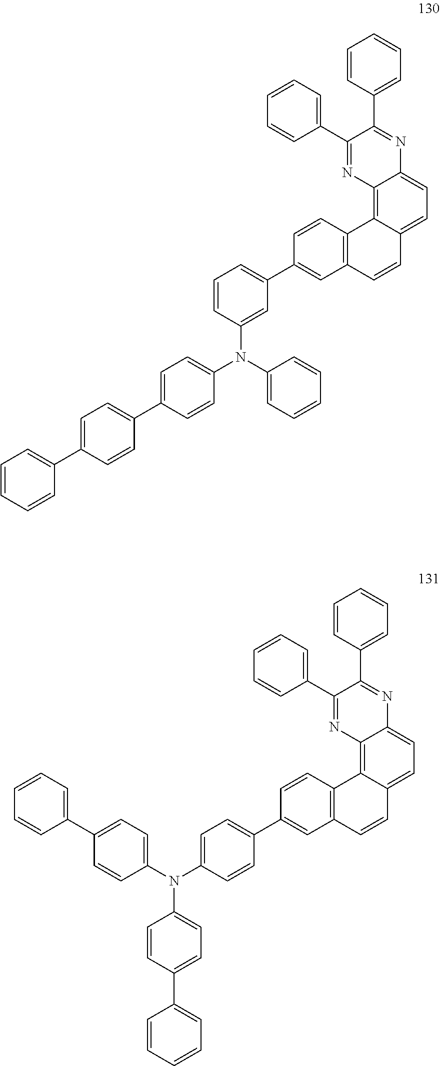 [SMILES: c1ccc(-c2ccc(-c3ccc(N(c4ccccc4)c4cccc(-c5ccc6c(ccc7ccc8nc(-c9ccccc9)c(-c9ccccc9)nc8c76)c5)c4)cc3)cc2)cc1 ; c1ccc(-c2ccc(N(c3ccc(-c4ccccc4)cc3)c3ccc(-c4ccc5c(ccc6ccc7nc(-c8ccccc8)c(-c8ccccc8)nc7c65)c4)cc3)cc2)cc1]

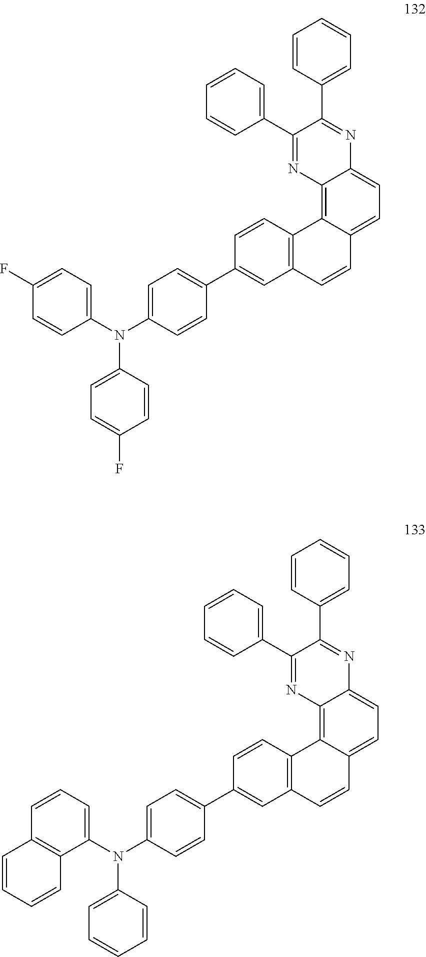 [SMILES: Fc1ccc(N(c2ccc(F)cc2)c2ccc(-c3ccc4c(ccc5ccc6nc(-c7ccccc7)c(-c7ccccc7)nc6c54)c3)cc2)cc1 ; c1ccc(-c2nc3ccc4ccc5cc(-c6ccc(N(c7ccccc7)c7cccc8ccccc78)cc6)ccc5c4c3nc2-c2ccccc2)cc1]

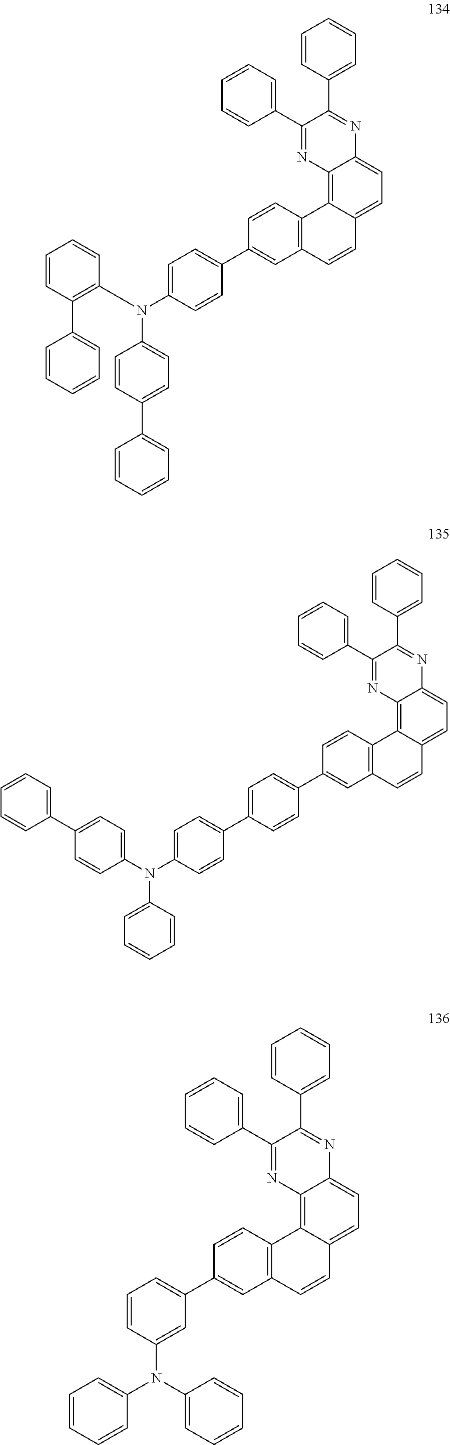 [SMILES: c1ccc(-c2ccc(N(c3ccc(-c4ccc5c(ccc6ccc7nc(-c8ccccc8)c(-c8ccccc8)nc7c65)c4)cc3)c3ccccc3-c3ccccc3)cc2)cc1 ; c1ccc(-c2ccc(N(c3ccccc3)c3ccc(-c4ccc(-c5ccc6c(ccc7ccc8nc(-c9ccccc9)c(-c9ccccc9)nc8c76)c5)cc4)cc3)cc2)cc1 ; c1ccc(-c2nc3ccc4ccc5cc(-c6cccc(N(c7ccccc7)c7ccccc7)c6)ccc5c4c3nc2-c2ccccc2)cc1]

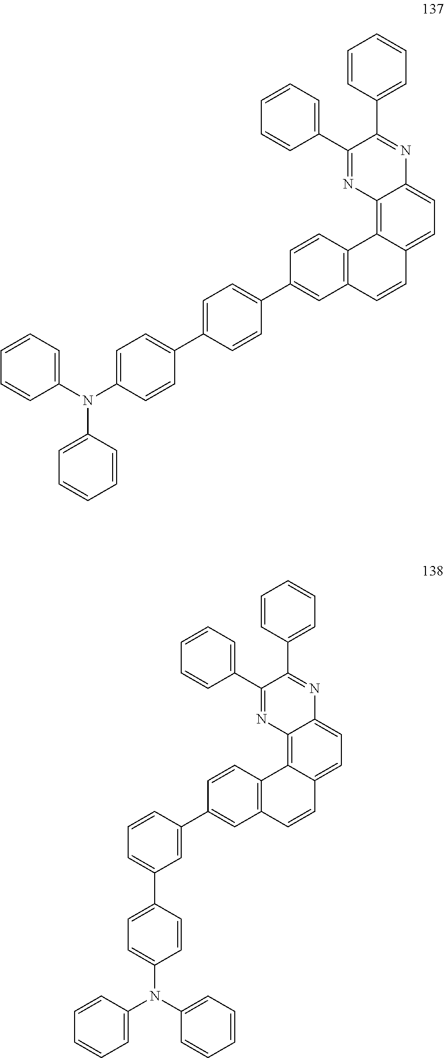 [SMILES: c1ccc(-c2nc3ccc4ccc5cc(-c6ccc(-c7ccc(N(c8ccccc8)c8ccccc8)cc7)cc6)ccc5c4c3nc2-c2ccccc2)cc1 ; c1ccc(-c2nc3ccc4ccc5cc(-c6cccc(-c7ccc(N(c8ccccc8)c8ccccc8)cc7)c6)ccc5c4c3nc2-c2ccccc2)cc1]

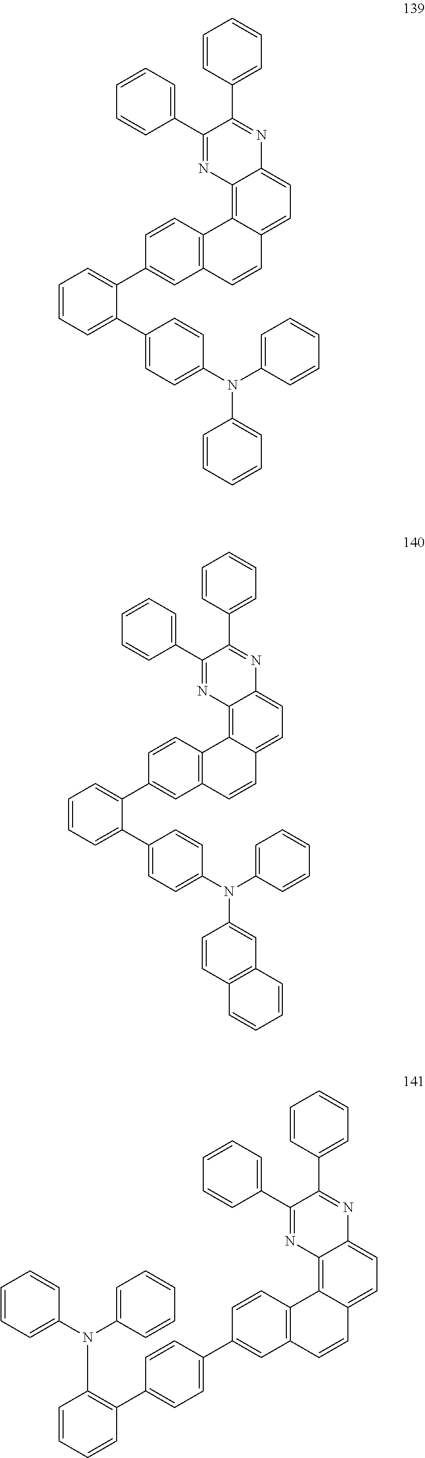 [SMILES: c1ccc(-c2nc3ccc4ccc5cc(-c6ccccc6-c6ccc(N(c7ccccc7)c7ccccc7)cc6)ccc5c4c3nc2-c2ccccc2)cc1 ; c1ccc(-c2nc3ccc4ccc5cc(-c6ccccc6-c6ccc(N(c7ccccc7)c7ccc8ccccc8c7)cc6)ccc5c4c3nc2-c2ccccc2)cc1 ; c1ccc(-c2nc3ccc4ccc5cc(-c6ccc(-c7ccccc7N(c7ccccc7)c7ccccc7)cc6)ccc5c4c3nc2-c2ccccc2)cc1]

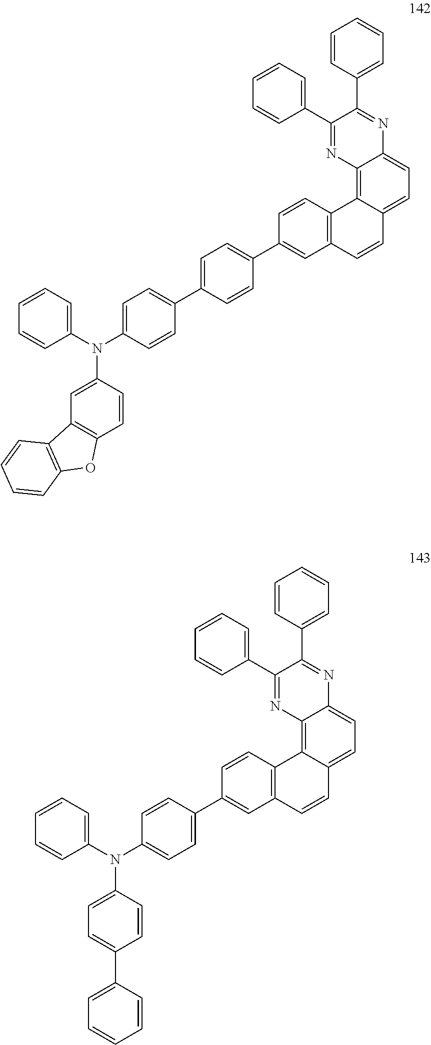 [SMILES: c1ccc(-c2nc3ccc4ccc5cc(-c6ccc(-c7ccc(N(c8ccccc8)c8ccc9oc%10ccccc%10c9c8)cc7)cc6)ccc5c4c3nc2-c2ccccc2)cc1 ; c1ccc(-c2ccc(N(c3ccccc3)c3ccc(-c4ccc5c(ccc6ccc7nc(-c8ccccc8)c(-c8ccccc8)nc7c65)c4)cc3)cc2)cc1]

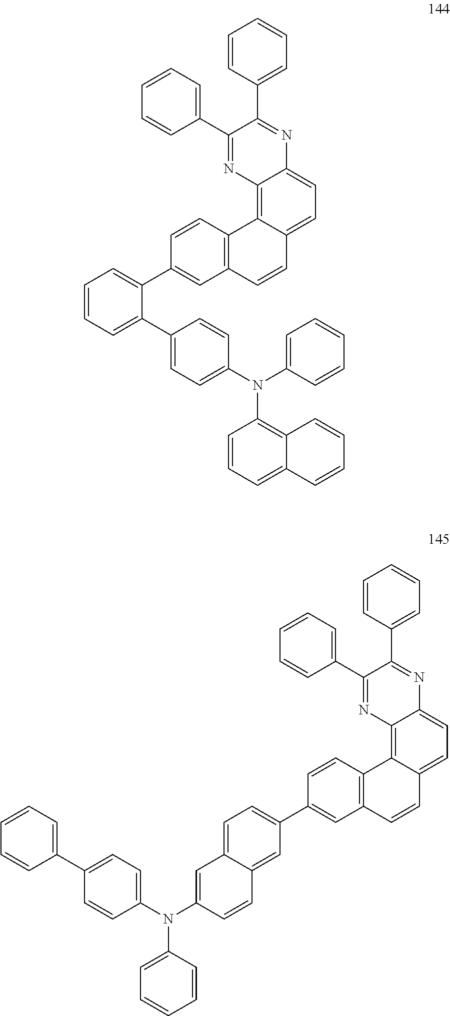 [SMILES: c1ccc(-c2nc3ccc4ccc5cc(-c6ccccc6-c6ccc(N(c7ccccc7)c7cccc8ccccc78)cc6)ccc5c4c3nc2-c2ccccc2)cc1 ; c1ccc(-c2ccc(N(c3ccccc3)c3ccc4cc(-c5ccc6c(ccc7ccc8nc(-c9ccccc9)c(-c9ccccc9)nc8c76)c5)ccc4c3)cc2)cc1]

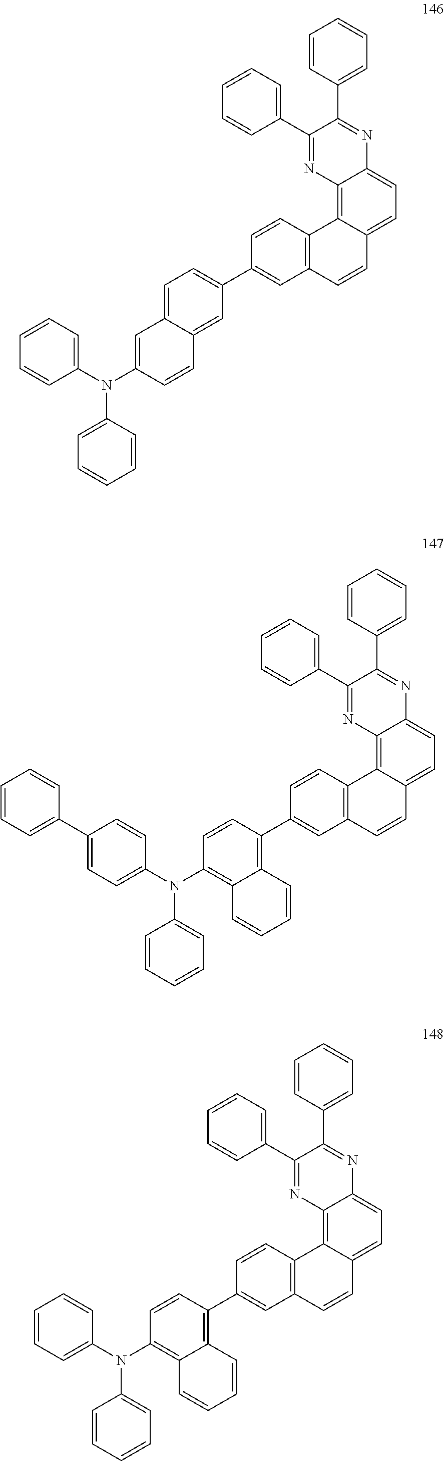 [SMILES: c1ccc(-c2nc3ccc4ccc5cc(-c6ccc7cc(N(c8ccccc8)c8ccccc8)ccc7c6)ccc5c4c3nc2-c2ccccc2)cc1 ; c1ccc(-c2ccc(N(c3ccccc3)c3ccc(-c4ccc5c(ccc6ccc7nc(-c8ccccc8)c(-c8ccccc8)nc7c65)c4)c4ccccc34)cc2)cc1 ; c1ccc(-c2nc3ccc4ccc5cc(-c6ccc(N(c7ccccc7)c7ccccc7)c7ccccc67)ccc5c4c3nc2-c2ccccc2)cc1]

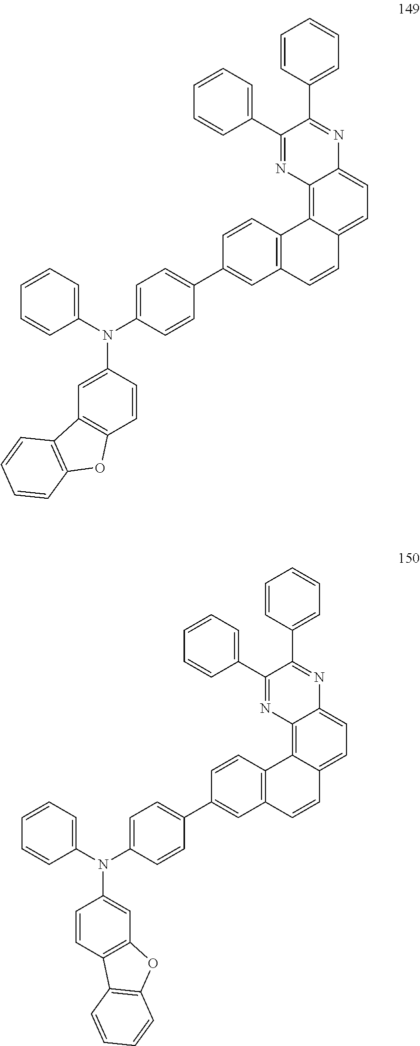 [SMILES: c1ccc(-c2nc3ccc4ccc5cc(-c6ccc(N(c7ccccc7)c7ccc8oc9ccccc9c8c7)cc6)ccc5c4c3nc2-c2ccccc2)cc1 ; c1ccc(-c2nc3ccc4ccc5cc(-c6ccc(N(c7ccccc7)c7ccc8c(c7)oc7ccccc78)cc6)ccc5c4c3nc2-c2ccccc2)cc1]

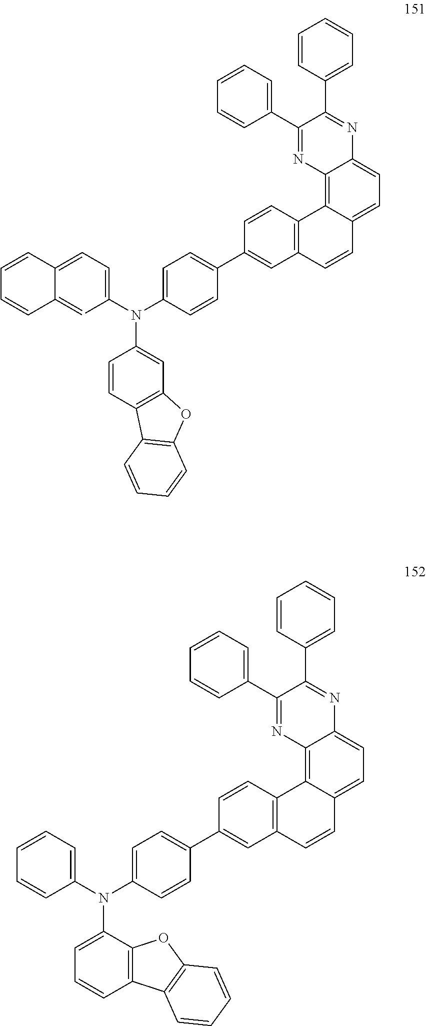

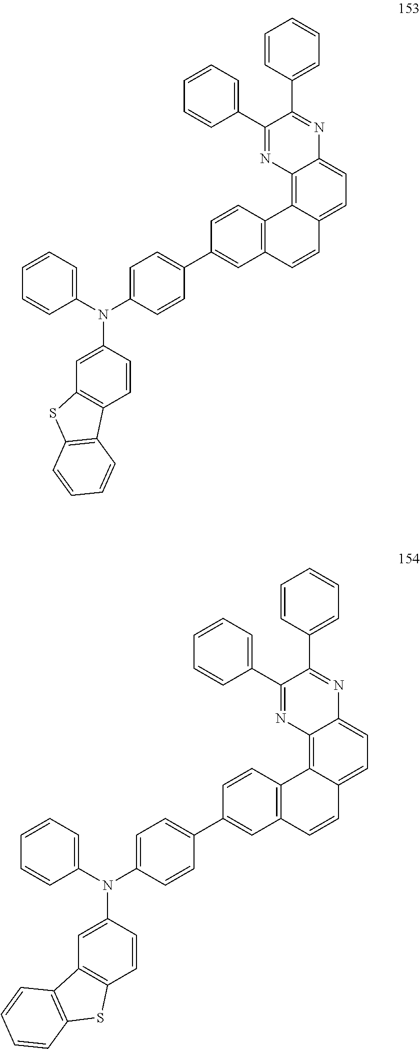 [SMILES: c1ccc(-c2nc3ccc4ccc5cc(-c6ccc(N(c7ccccc7)c7ccc8c(c7)sc7ccccc78)cc6)ccc5c4c3nc2-c2ccccc2)cc1 ; c1ccc(-c2nc3ccc4ccc5cc(-c6ccc(N(c7ccccc7)c7ccc8sc9ccccc9c8c7)cc6)ccc5c4c3nc2-c2ccccc2)cc1]

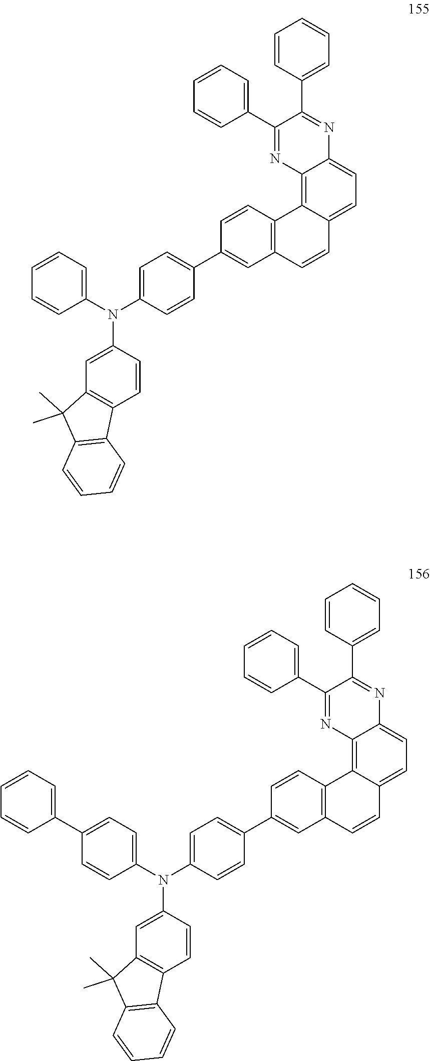 [SMILES: CC1(C)c2ccccc2-c2ccc(N(c3ccccc3)c3ccc(-c4ccc5c(ccc6ccc7nc(-c8ccccc8)c(-c8ccccc8)nc7c65)c4)cc3)cc21 ; CC1(C)c2ccccc2-c2ccc(N(c3ccc(-c4ccccc4)cc3)c3ccc(-c4ccc5c(ccc6ccc7nc(-c8ccccc8)c(-c8ccccc8)nc7c65)c4)cc3)cc21]

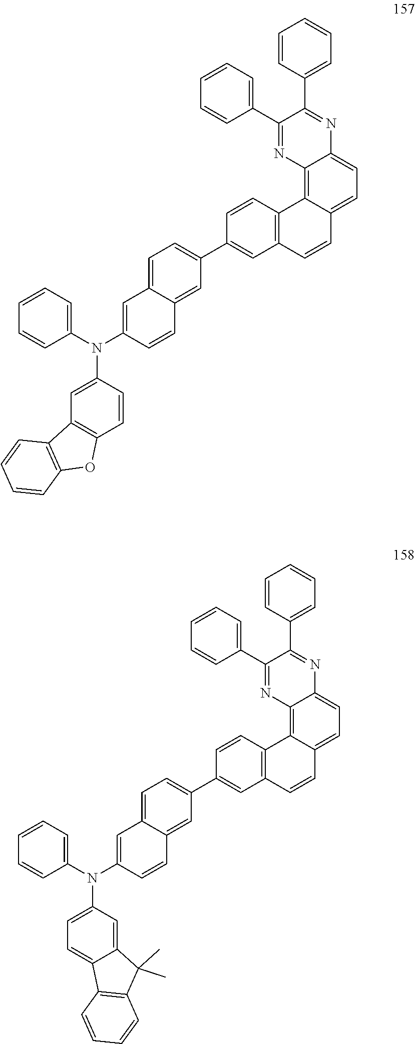 [SMILES: c1ccc(-c2nc3ccc4ccc5cc(-c6ccc7cc(N(c8ccccc8)c8ccc9oc%10ccccc%10c9c8)ccc7c6)ccc5c4c3nc2-c2ccccc2)cc1 ; CC1(C)c2ccccc2-c2ccc(N(c3ccccc3)c3ccc4cc(-c5ccc6c(ccc7ccc8nc(-c9ccccc9)c(-c9ccccc9)nc8c76)c5)ccc4c3)cc21]

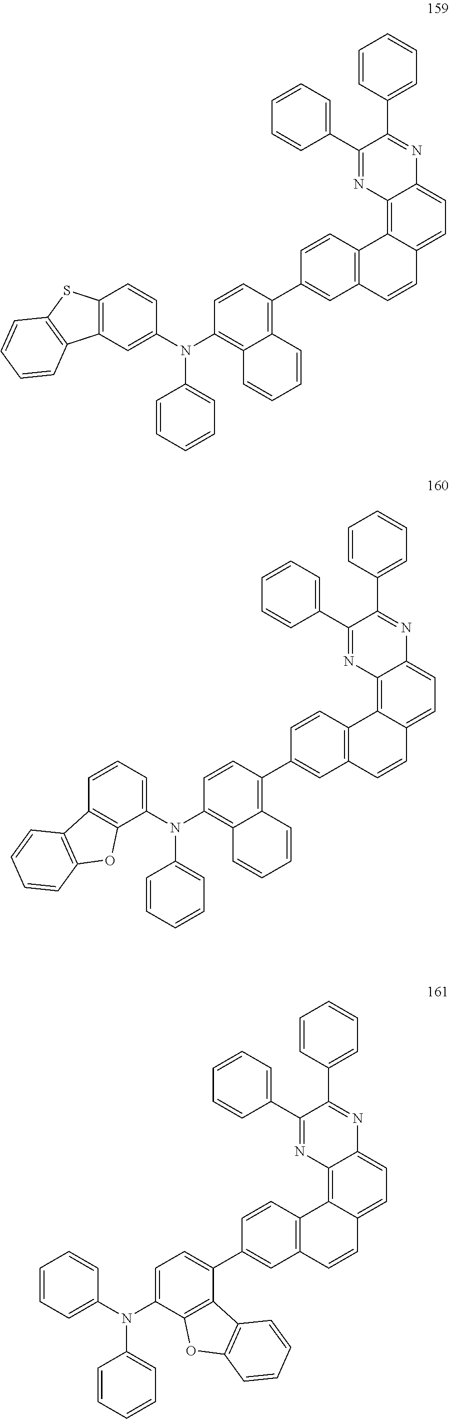 [SMILES: c1ccc(-c2nc3ccc4ccc5cc(-c6ccc(N(c7ccccc7)c7ccc8sc9ccccc9c8c7)c7ccccc67)ccc5c4c3nc2-c2ccccc2)cc1 ; c1ccc(-c2nc3ccc4ccc5cc(-c6ccc(N(c7ccccc7)c7cccc8c7oc7ccccc78)c7ccccc67)ccc5c4c3nc2-c2ccccc2)cc1 ; c1ccc(-c2nc3ccc4ccc5cc(-c6ccc(N(c7ccccc7)c7ccccc7)c7oc8ccccc8c67)ccc5c4c3nc2-c2ccccc2)cc1]

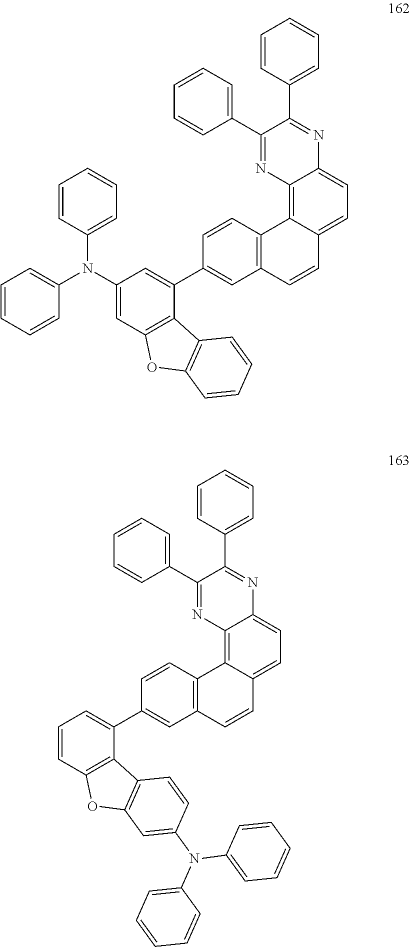 [SMILES: c1ccc(-c2nc3ccc4ccc5cc(-c6cc(N(c7ccccc7)c7ccccc7)cc7oc8ccccc8c67)ccc5c4c3nc2-c2ccccc2)cc1 ; c1ccc(-c2nc3ccc4ccc5cc(-c6cccc7oc8cc(N(c9ccccc9)c9ccccc9)ccc8c67)ccc5c4c3nc2-c2ccccc2)cc1]

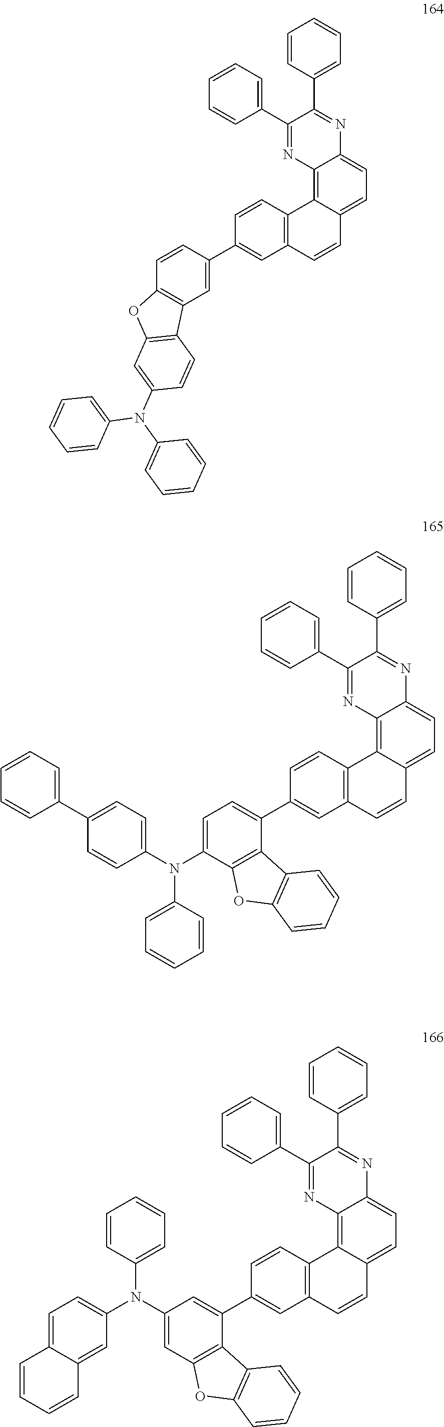 [SMILES: c1ccc(-c2nc3ccc4ccc5cc(-c6ccc7oc8cc(N(c9ccccc9)c9ccccc9)ccc8c7c6)ccc5c4c3nc2-c2ccccc2)cc1 ; c1ccc(-c2ccc(N(c3ccccc3)c3ccc(-c4ccc5c(ccc6ccc7nc(-c8ccccc8)c(-c8ccccc8)nc7c65)c4)c4c3oc3ccccc34)cc2)cc1 ; c1ccc(-c2nc3ccc4ccc5cc(-c6cc(N(c7ccccc7)c7ccc8ccccc8c7)cc7oc8ccccc8c67)ccc5c4c3nc2-c2ccccc2)cc1]

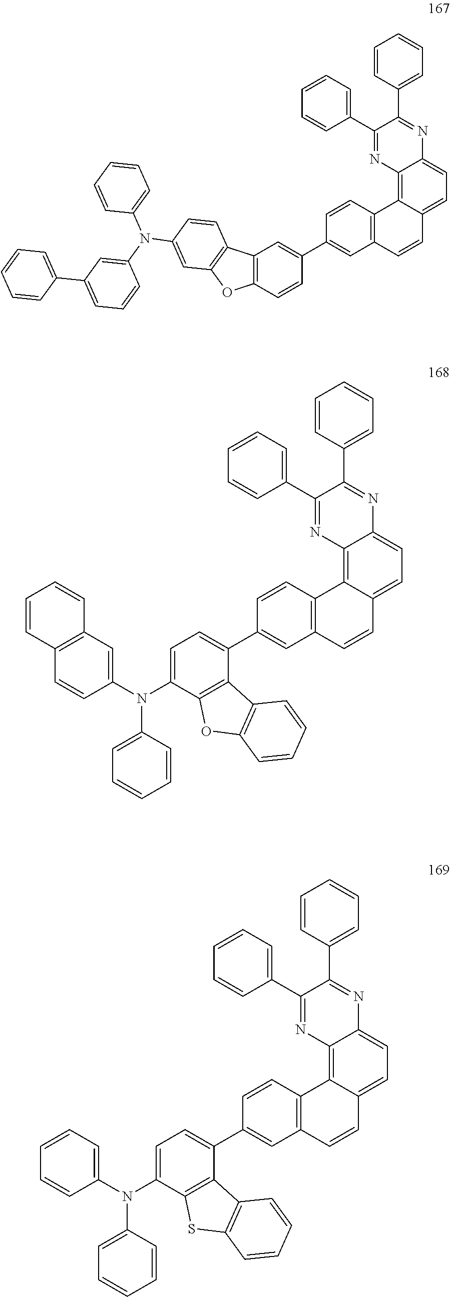 [SMILES: c1ccc(-c2cccc(N(c3ccccc3)c3ccc4c(c3)oc3ccc(-c5ccc6c(ccc7ccc8nc(-c9ccccc9)c(-c9ccccc9)nc8c76)c5)cc34)c2)cc1 ; c1ccc(-c2nc3ccc4ccc5cc(-c6ccc(N(c7ccccc7)c7ccc8ccccc8c7)c7oc8ccccc8c67)ccc5c4c3nc2-c2ccccc2)cc1 ; c1ccc(-c2nc3ccc4ccc5cc(-c6ccc(N(c7ccccc7)c7ccccc7)c7sc8ccccc8c67)ccc5c4c3nc2-c2ccccc2)cc1]

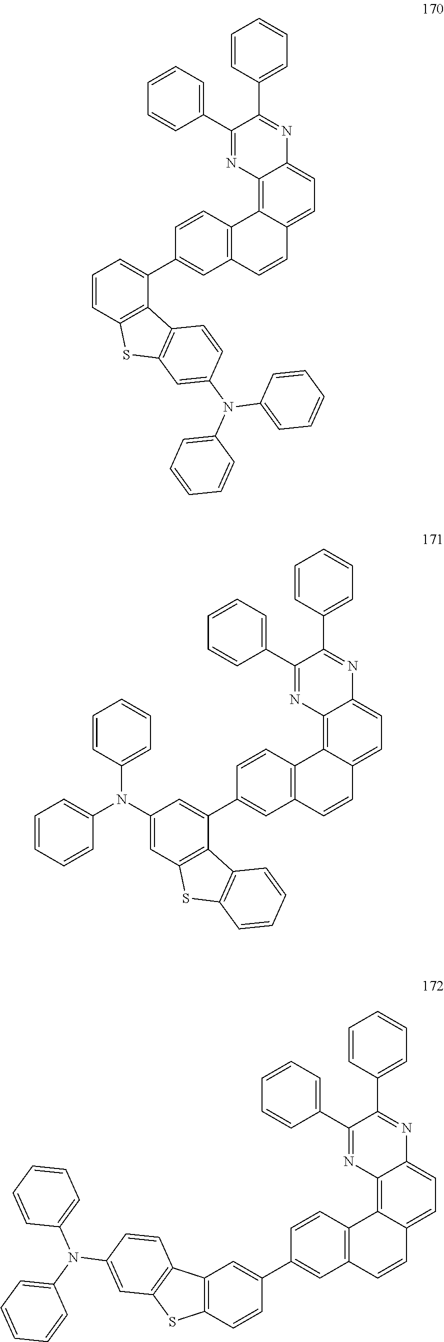 [SMILES: c1ccc(-c2nc3ccc4ccc5cc(-c6cccc7sc8cc(N(c9ccccc9)c9ccccc9)ccc8c67)ccc5c4c3nc2-c2ccccc2)cc1 ; c1ccc(-c2nc3ccc4ccc5cc(-c6cc(N(c7ccccc7)c7ccccc7)cc7sc8ccccc8c67)ccc5c4c3nc2-c2ccccc2)cc1 ; c1ccc(-c2nc3ccc4ccc5cc(-c6ccc7sc8cc(N(c9ccccc9)c9ccccc9)ccc8c7c6)ccc5c4c3nc2-c2ccccc2)cc1]

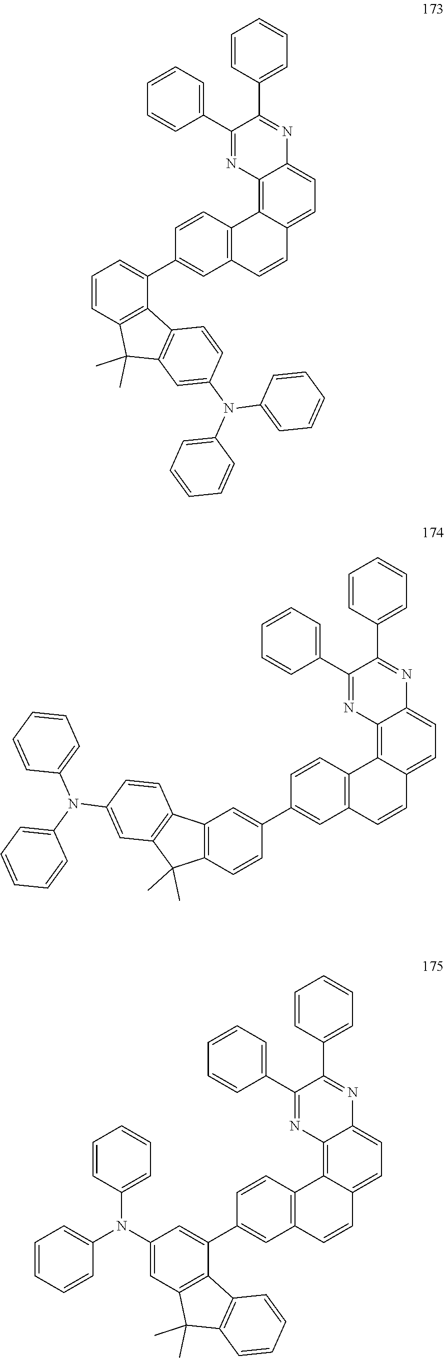 [SMILES: CC1(C)c2cc(N(c3ccccc3)c3ccccc3)ccc2-c2c(-c3ccc4c(ccc5ccc6nc(-c7ccccc7)c(-c7ccccc7)nc6c54)c3)cccc21 ; CC1(C)c2ccc(-c3ccc4c(ccc5ccc6nc(-c7ccccc7)c(-c7ccccc7)nc6c54)c3)cc2-c2ccc(N(c3ccccc3)c3ccccc3)cc21 ; CC1(C)c2ccccc2-c2c(-c3ccc4c(ccc5ccc6nc(-c7ccccc7)c(-c7ccccc7)nc6c54)c3)cc(N(c3ccccc3)c3ccccc3)cc21]

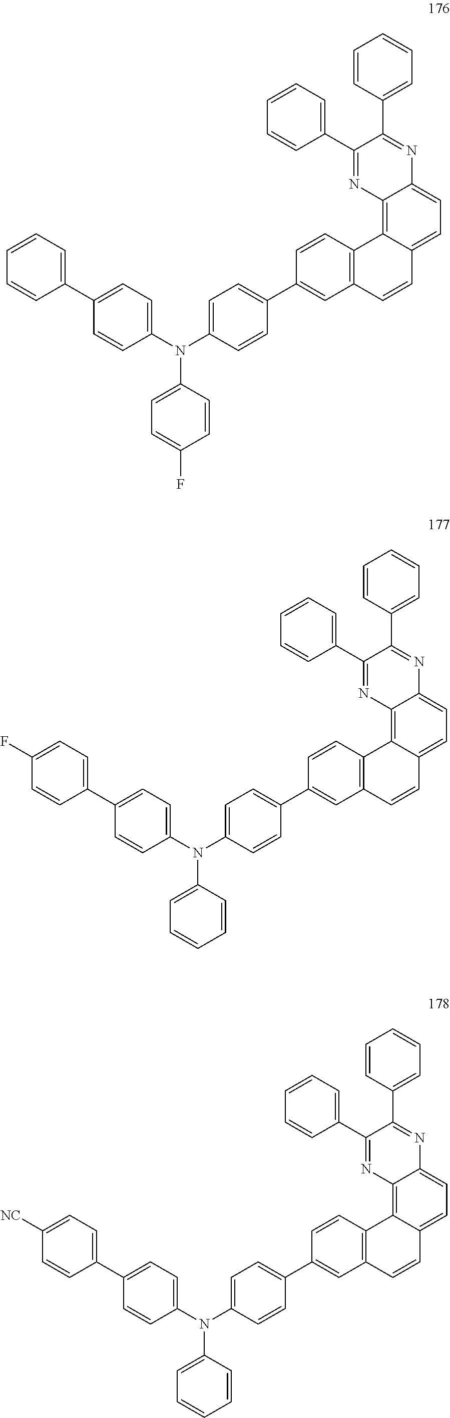 [SMILES: Fc1ccc(N(c2ccc(-c3ccccc3)cc2)c2ccc(-c3ccc4c(ccc5ccc6nc(-c7ccccc7)c(-c7ccccc7)nc6c54)c3)cc2)cc1 ; Fc1ccc(-c2ccc(N(c3ccccc3)c3ccc(-c4ccc5c(ccc6ccc7nc(-c8ccccc8)c(-c8ccccc8)nc7c65)c4)cc3)cc2)cc1 ; N#Cc1ccc(-c2ccc(N(c3ccccc3)c3ccc(-c4ccc5c(ccc6ccc7nc(-c8ccccc8)c(-c8ccccc8)nc7c65)c4)cc3)cc2)cc1]

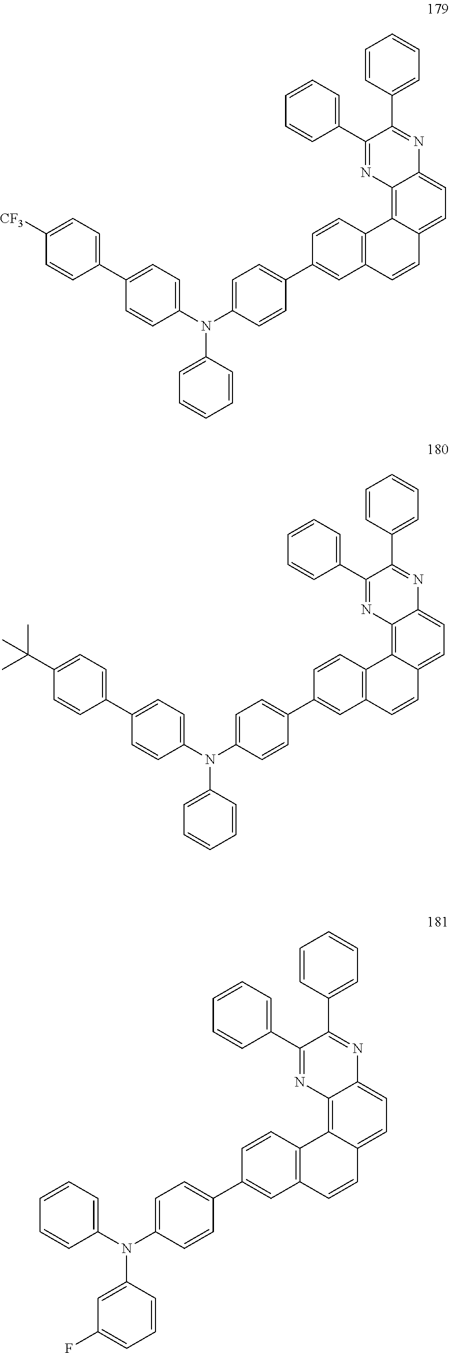 [SMILES: FC(F)(F)c1ccc(-c2ccc(N(c3ccccc3)c3ccc(-c4ccc5c(ccc6ccc7nc(-c8ccccc8)c(-c8ccccc8)nc7c65)c4)cc3)cc2)cc1 ; CC(C)(C)c1ccc(-c2ccc(N(c3ccccc3)c3ccc(-c4ccc5c(ccc6ccc7nc(-c8ccccc8)c(-c8ccccc8)nc7c65)c4)cc3)cc2)cc1 ; Fc1cccc(N(c2ccccc2)c2ccc(-c3ccc4c(ccc5ccc6nc(-c7ccccc7)c(-c7ccccc7)nc6c54)c3)cc2)c1]

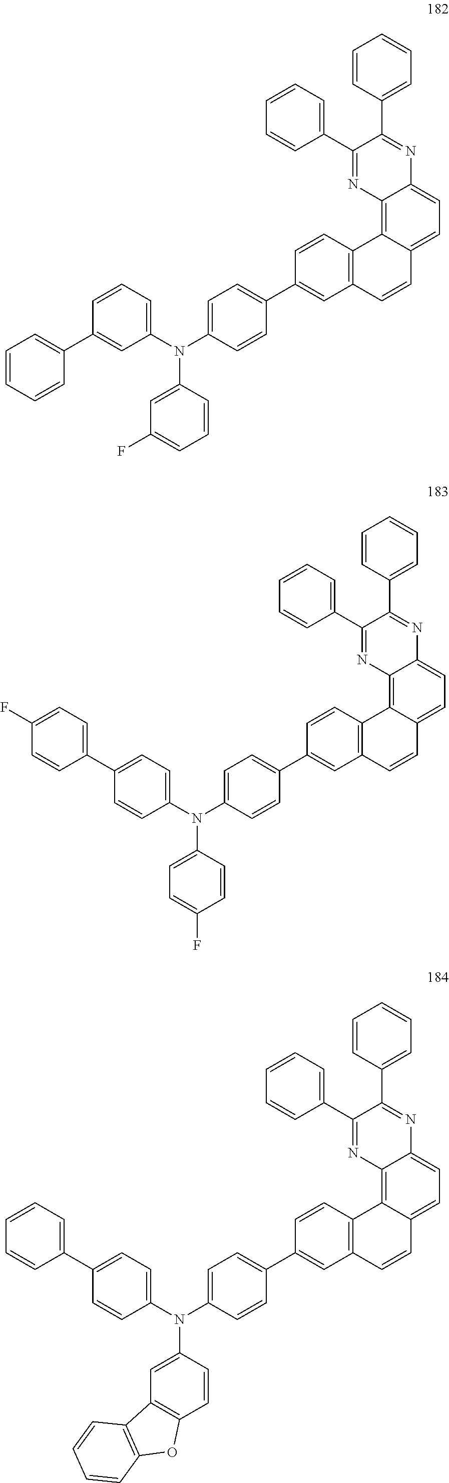 [SMILES: Fc1cccc(N(c2ccc(-c3ccc4c(ccc5ccc6nc(-c7ccccc7)c(-c7ccccc7)nc6c54)c3)cc2)c2cccc(-c3ccccc3)c2)c1 ; Fc1ccc(-c2ccc(N(c3ccc(F)cc3)c3ccc(-c4ccc5c(ccc6ccc7nc(-c8ccccc8)c(-c8ccccc8)nc7c65)c4)cc3)cc2)cc1 ; c1ccc(-c2ccc(N(c3ccc(-c4ccc5c(ccc6ccc7nc(-c8ccccc8)c(-c8ccccc8)nc7c65)c4)cc3)c3ccc4oc5ccccc5c4c3)cc2)cc1]

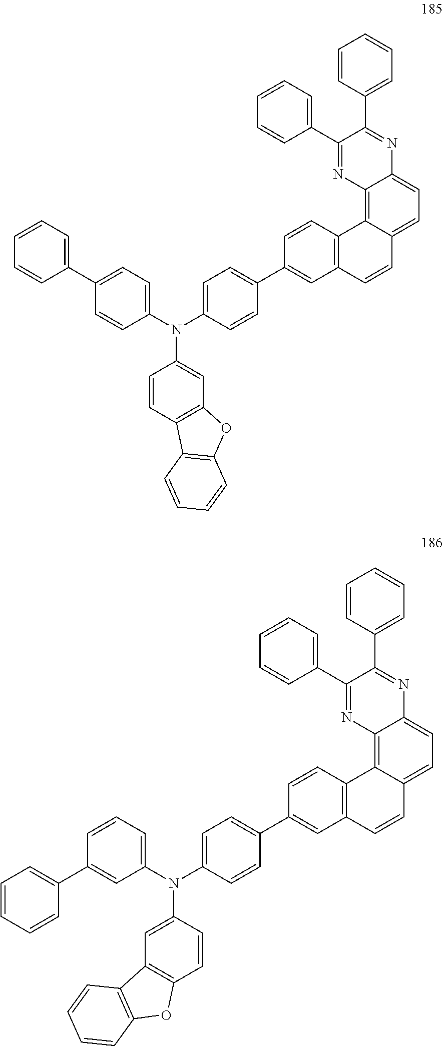 [SMILES: c1ccc(-c2ccc(N(c3ccc(-c4ccc5c(ccc6ccc7nc(-c8ccccc8)c(-c8ccccc8)nc7c65)c4)cc3)c3ccc4c(c3)oc3ccccc34)cc2)cc1 ; c1ccc(-c2cccc(N(c3ccc(-c4ccc5c(ccc6ccc7nc(-c8ccccc8)c(-c8ccccc8)nc7c65)c4)cc3)c3ccc4oc5ccccc5c4c3)c2)cc1]

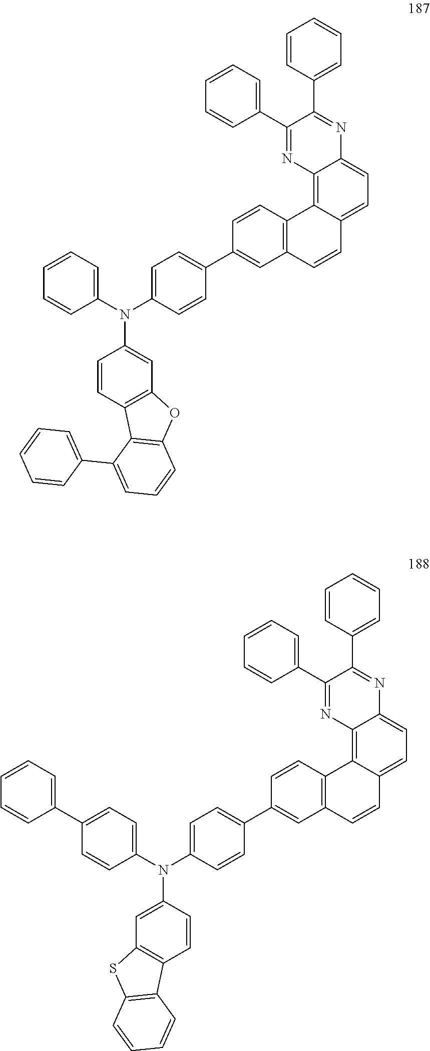 [SMILES: c1ccc(-c2nc3ccc4ccc5cc(-c6ccc(N(c7ccccc7)c7ccc8c(c7)oc7cccc(-c9ccccc9)c78)cc6)ccc5c4c3nc2-c2ccccc2)cc1 ; c1ccc(-c2ccc(N(c3ccc(-c4ccc5c(ccc6ccc7nc(-c8ccccc8)c(-c8ccccc8)nc7c65)c4)cc3)c3ccc4c(c3)sc3ccccc34)cc2)cc1]

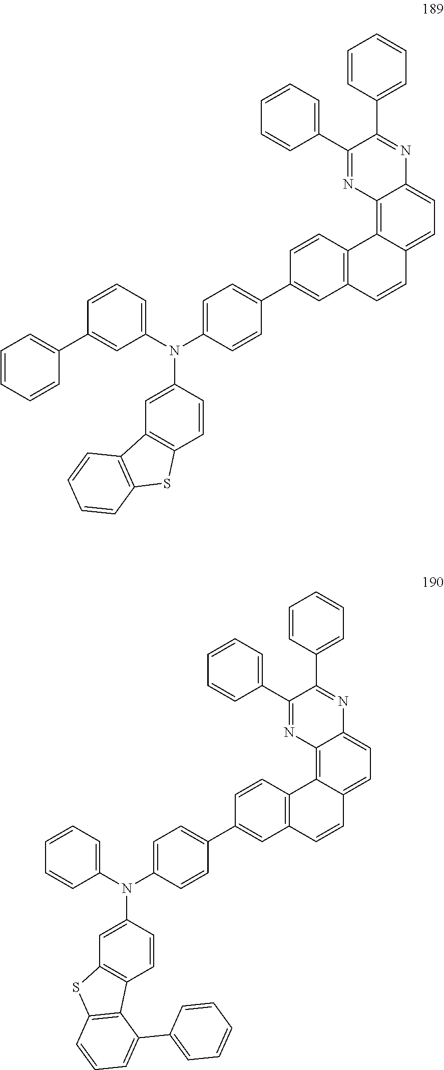 [SMILES: c1ccc(-c2cccc(N(c3ccc(-c4ccc5c(ccc6ccc7nc(-c8ccccc8)c(-c8ccccc8)nc7c65)c4)cc3)c3ccc4sc5ccccc5c4c3)c2)cc1 ; c1ccc(-c2nc3ccc4ccc5cc(-c6ccc(N(c7ccccc7)c7ccc8c(c7)sc7cccc(-c9ccccc9)c78)cc6)ccc5c4c3nc2-c2ccccc2)cc1]

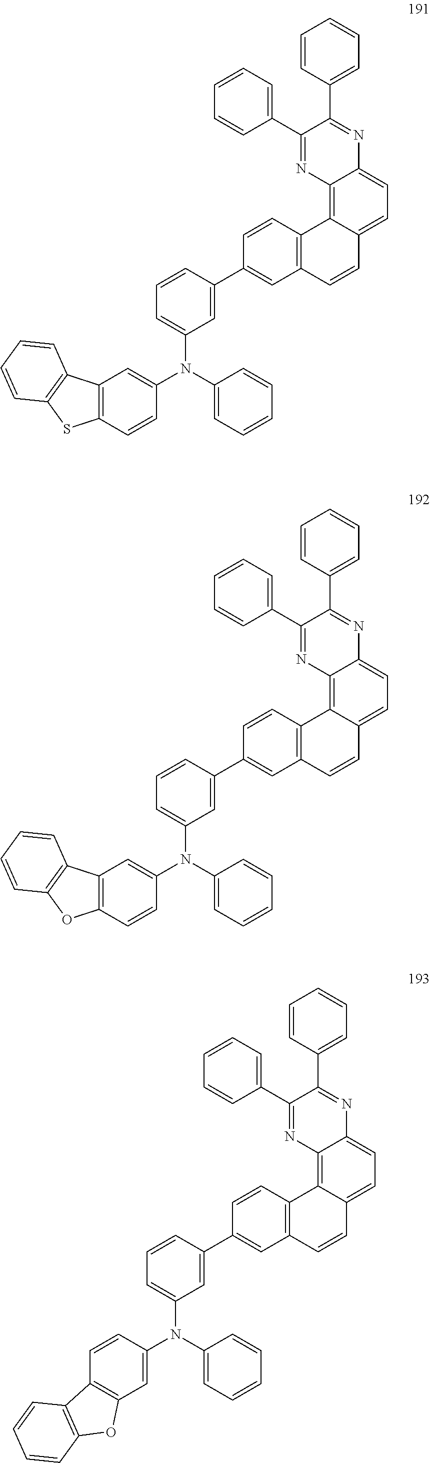 [SMILES: c1ccc(-c2nc3ccc4ccc5cc(-c6cccc(N(c7ccccc7)c7ccc8sc9ccccc9c8c7)c6)ccc5c4c3nc2-c2ccccc2)cc1 ; c1ccc(-c2nc3ccc4ccc5cc(-c6cccc(N(c7ccccc7)c7ccc8oc9ccccc9c8c7)c6)ccc5c4c3nc2-c2ccccc2)cc1 ; c1ccc(-c2nc3ccc4ccc5cc(-c6cccc(N(c7ccccc7)c7ccc8c(c7)oc7ccccc78)c6)ccc5c4c3nc2-c2ccccc2)cc1]

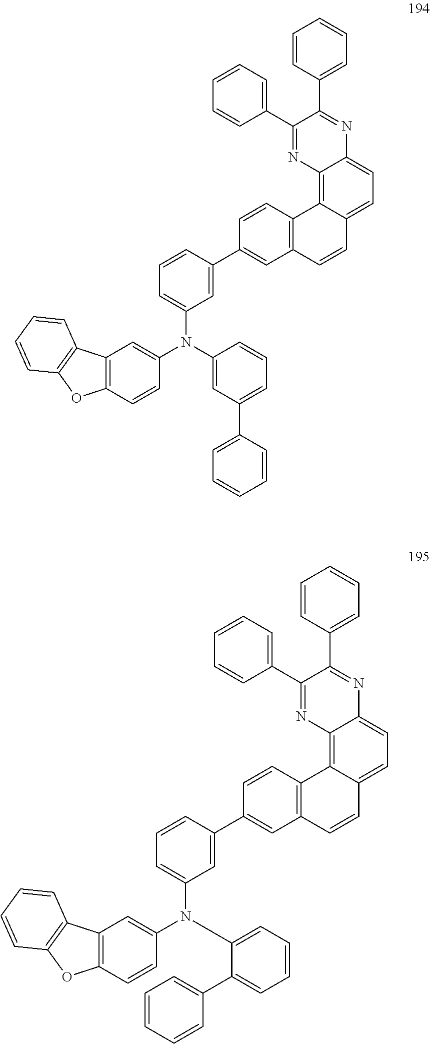 [SMILES: c1ccc(-c2cccc(N(c3cccc(-c4ccc5c(ccc6ccc7nc(-c8ccccc8)c(-c8ccccc8)nc7c65)c4)c3)c3ccc4oc5ccccc5c4c3)c2)cc1 ; c1ccc(-c2ccccc2N(c2cccc(-c3ccc4c(ccc5ccc6nc(-c7ccccc7)c(-c7ccccc7)nc6c54)c3)c2)c2ccc3oc4ccccc4c3c2)cc1]

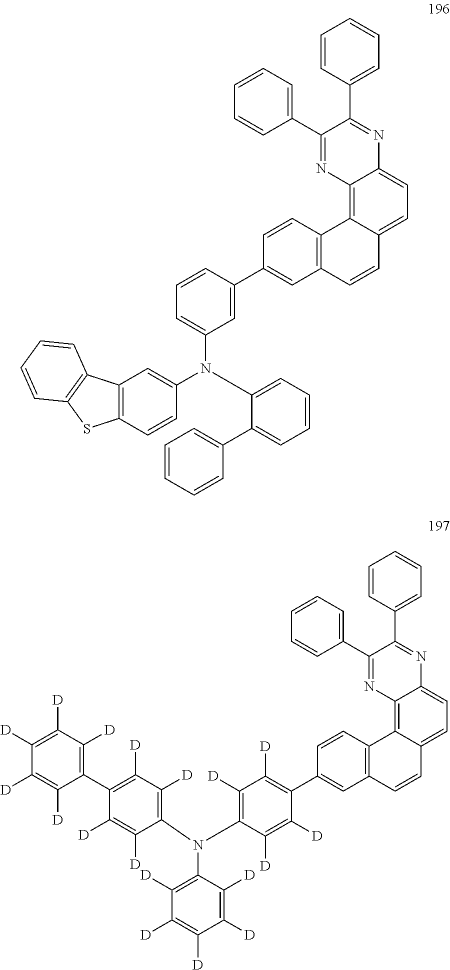 [SMILES: c1ccc(-c2ccccc2N(c2cccc(-c3ccc4c(ccc5ccc6nc(-c7ccccc7)c(-c7ccccc7)nc6c54)c3)c2)c2ccc3sc4ccccc4c3c2)cc1 ; [2H]c1c([2H])c([2H])c(-c2c([2H])c([2H])c(N(c3c([2H])c([2H])c([2H])c([2H])c3[2H])c3c([2H])c([2H])c(-c4ccc5c(ccc6ccc7nc(-c8ccccc8)c(-c8ccccc8)nc7c65)c4)c([2H])c3[2H])c([2H])c2[2H])c([2H])c1[2H]]

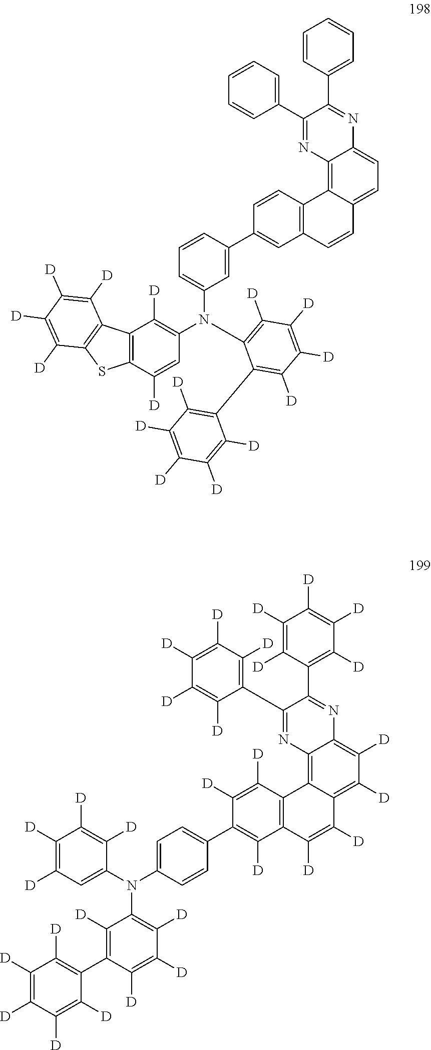 [SMILES: [2H]c1c([2H])c([2H])c(-c2c([2H])c([2H])c([2H])c([2H])c2N(c2cccc(-c3ccc4c(ccc5ccc6nc(-c7ccccc7)c(-c7ccccc7)nc6c54)c3)c2)c2cc([2H])c3sc4c([2H])c([2H])c([2H])c([2H])c4c3c2[2H])c([2H])c1[2H] ; [2H]c1cc(N(c2ccc(-c3c([2H])c([2H])c4c(c3[2H])c([2H])c([2H])c3c([2H])c([2H])c5nc(-c6c([2H])c([2H])c([2H])c([2H])c6[2H])c(-c6c([2H])c([2H])c([2H])c([2H])c6[2H])nc5c34)cc2)c2c([2H])c([2H])c([2H])c(-c3c([2H])c([2H])c([2H])c([2H])c3[2H])c2[2H])c([2H])c([2H])c1[2H]]

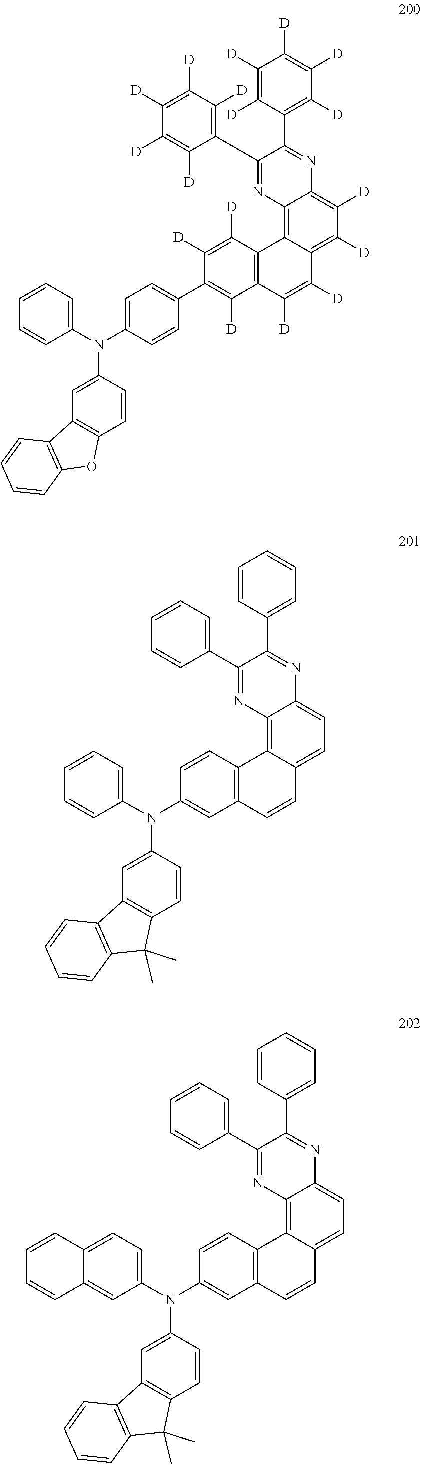 [SMILES: [2H]c1c([2H])c([2H])c(-c2nc3c([2H])c([2H])c4c([2H])c([2H])c5c([2H])c(-c6ccc(N(c7ccccc7)c7ccc8oc9ccccc9c8c7)cc6)c([2H])c([2H])c5c4c3nc2-c2c([2H])c([2H])c([2H])c([2H])c2[2H])c([2H])c1[2H] ; CC1(C)c2ccccc2-c2cc(N(c3ccccc3)c3ccc4c(ccc5ccc6nc(-c7ccccc7)c(-c7ccccc7)nc6c54)c3)ccc21 ; CC1(C)c2ccccc2-c2cc(N(c3ccc4ccccc4c3)c3ccc4c(ccc5ccc6nc(-c7ccccc7)c(-c7ccccc7)nc6c54)c3)ccc21]

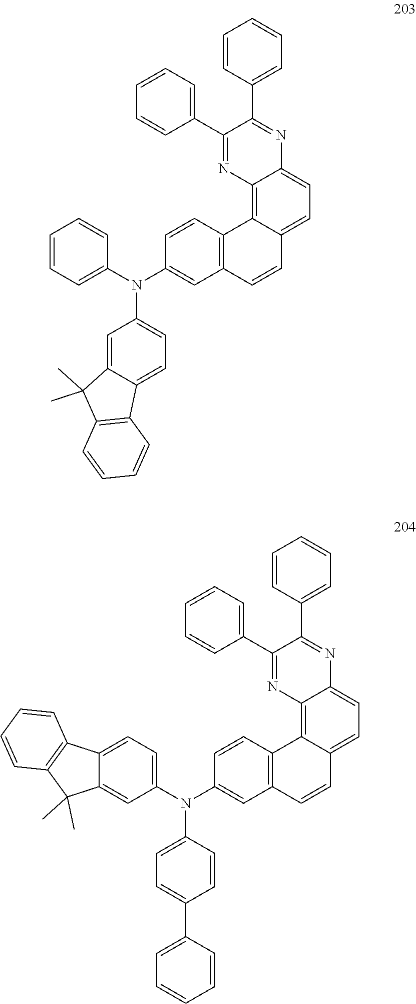 [SMILES: CC1(C)c2ccccc2-c2ccc(N(c3ccccc3)c3ccc4c(ccc5ccc6nc(-c7ccccc7)c(-c7ccccc7)nc6c54)c3)cc21 ; CC1(C)c2ccccc2-c2ccc(N(c3ccc(-c4ccccc4)cc3)c3ccc4c(ccc5ccc6nc(-c7ccccc7)c(-c7ccccc7)nc6c54)c3)cc21]

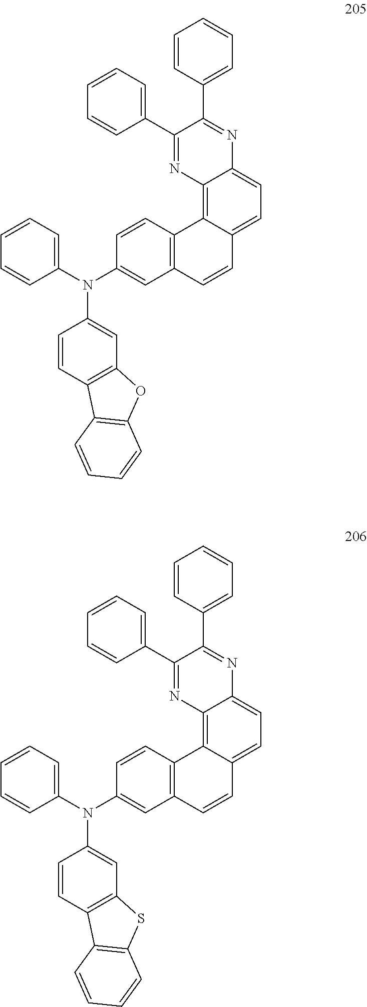 [SMILES: c1ccc(-c2nc3ccc4ccc5cc(N(c6ccccc6)c6ccc7c(c6)oc6ccccc67)ccc5c4c3nc2-c2ccccc2)cc1 ; c1ccc(-c2nc3ccc4ccc5cc(N(c6ccccc6)c6ccc7c(c6)sc6ccccc67)ccc5c4c3nc2-c2ccccc2)cc1]

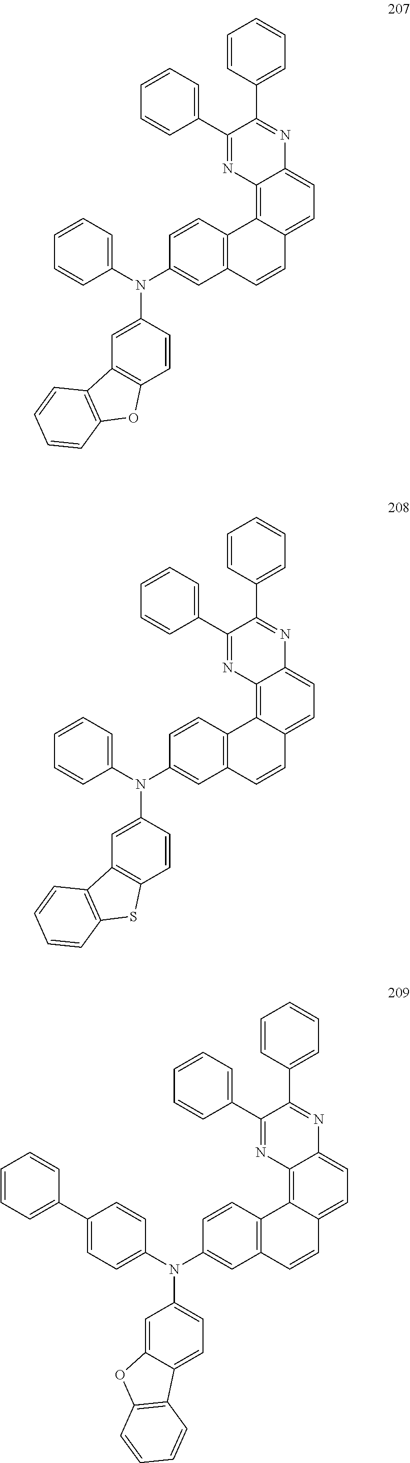 [SMILES: c1ccc(-c2nc3ccc4ccc5cc(N(c6ccccc6)c6ccc7oc8ccccc8c7c6)ccc5c4c3nc2-c2ccccc2)cc1 ; c1ccc(-c2nc3ccc4ccc5cc(N(c6ccccc6)c6ccc7sc8ccccc8c7c6)ccc5c4c3nc2-c2ccccc2)cc1 ; c1ccc(-c2ccc(N(c3ccc4c(ccc5ccc6nc(-c7ccccc7)c(-c7ccccc7)nc6c54)c3)c3ccc4c(c3)oc3ccccc34)cc2)cc1]

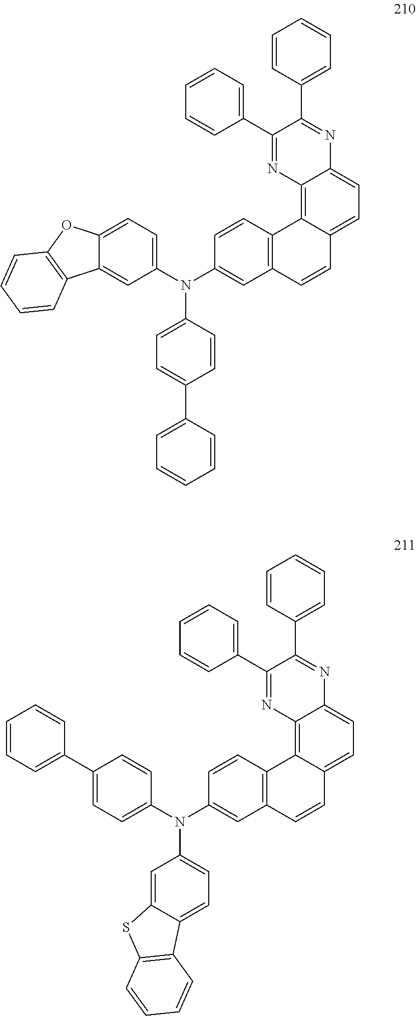 [SMILES: c1ccc(-c2ccc(N(c3ccc4c(ccc5ccc6nc(-c7ccccc7)c(-c7ccccc7)nc6c54)c3)c3ccc4oc5ccccc5c4c3)cc2)cc1 ; c1ccc(-c2ccc(N(c3ccc4c(ccc5ccc6nc(-c7ccccc7)c(-c7ccccc7)nc6c54)c3)c3ccc4c(c3)sc3ccccc34)cc2)cc1]

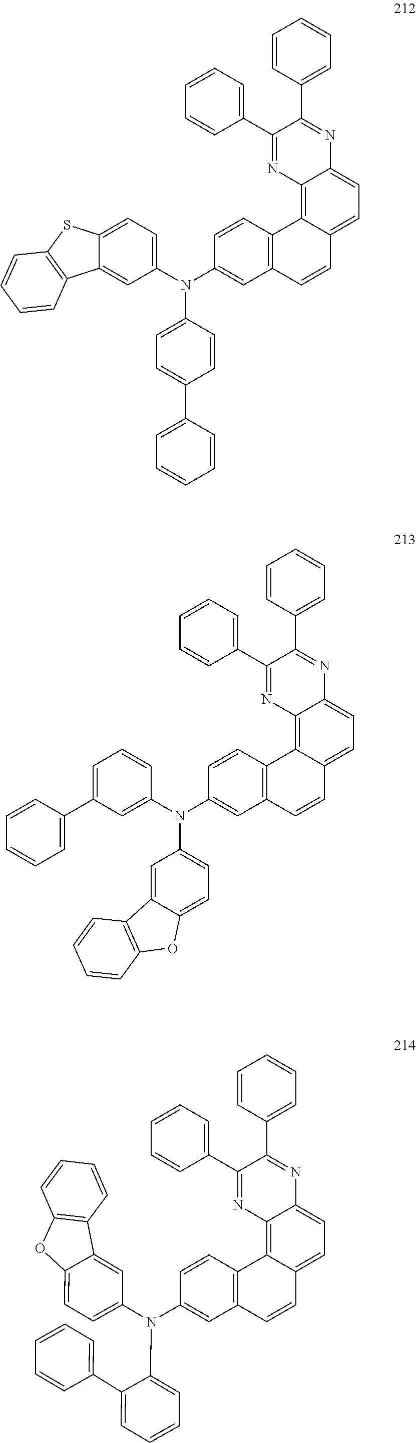 [SMILES: c1ccc(-c2ccc(N(c3ccc4c(ccc5ccc6nc(-c7ccccc7)c(-c7ccccc7)nc6c54)c3)c3ccc4sc5ccccc5c4c3)cc2)cc1 ; c1ccc(-c2cccc(N(c3ccc4c(ccc5ccc6nc(-c7ccccc7)c(-c7ccccc7)nc6c54)c3)c3ccc4oc5ccccc5c4c3)c2)cc1 ; c1ccc(-c2ccccc2N(c2ccc3c(ccc4ccc5nc(-c6ccccc6)c(-c6ccccc6)nc5c43)c2)c2ccc3oc4ccccc4c3c2)cc1]

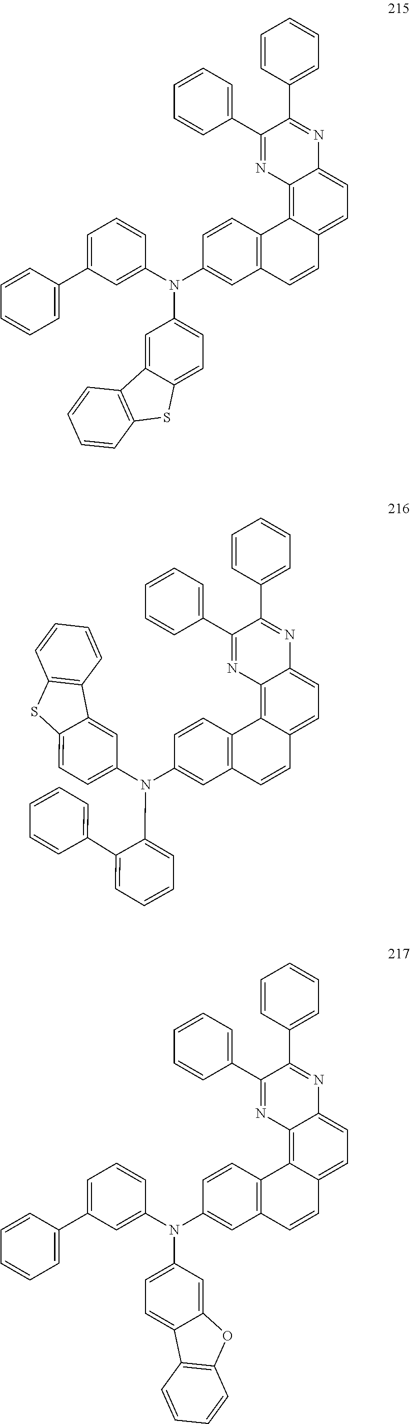 [SMILES: c1ccc(-c2cccc(N(c3ccc4c(ccc5ccc6nc(-c7ccccc7)c(-c7ccccc7)nc6c54)c3)c3ccc4sc5ccccc5c4c3)c2)cc1 ; c1ccc(-c2ccccc2N(c2ccc3c(ccc4ccc5nc(-c6ccccc6)c(-c6ccccc6)nc5c43)c2)c2ccc3sc4ccccc4c3c2)cc1 ; c1ccc(-c2cccc(N(c3ccc4c(ccc5ccc6nc(-c7ccccc7)c(-c7ccccc7)nc6c54)c3)c3ccc4c(c3)oc3ccccc34)c2)cc1]

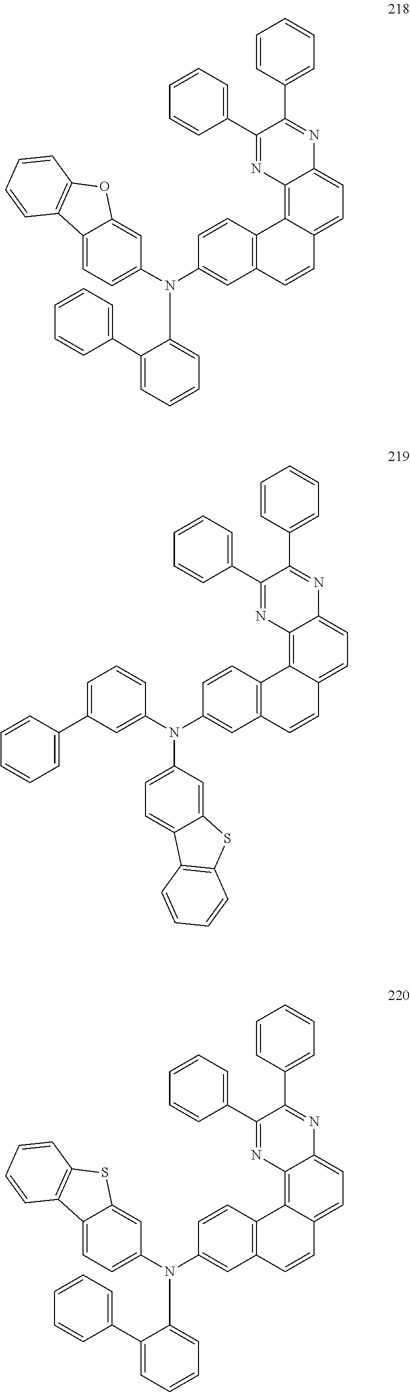 [SMILES: c1ccc(-c2ccccc2N(c2ccc3c(ccc4ccc5nc(-c6ccccc6)c(-c6ccccc6)nc5c43)c2)c2ccc3c(c2)oc2ccccc23)cc1 ; c1ccc(-c2cccc(N(c3ccc4c(ccc5ccc6nc(-c7ccccc7)c(-c7ccccc7)nc6c54)c3)c3ccc4c(c3)sc3ccccc34)c2)cc1 ; c1ccc(-c2ccccc2N(c2ccc3c(ccc4ccc5nc(-c6ccccc6)c(-c6ccccc6)nc5c43)c2)c2ccc3c(c2)sc2ccccc23)cc1]

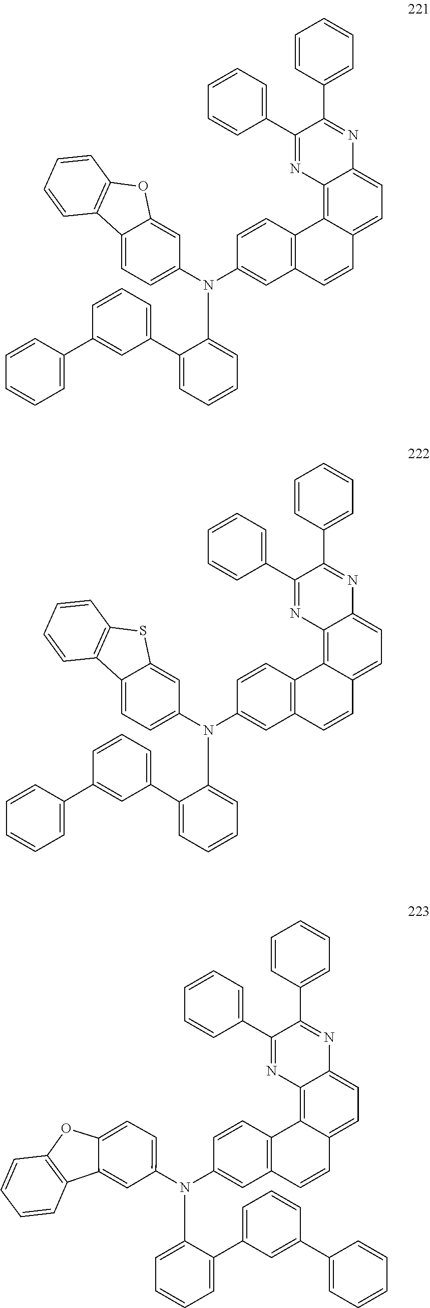 [SMILES: c1ccc(-c2cccc(-c3ccccc3N(c3ccc4c(ccc5ccc6nc(-c7ccccc7)c(-c7ccccc7)nc6c54)c3)c3ccc4c(c3)oc3ccccc34)c2)cc1 ; c1ccc(-c2cccc(-c3ccccc3N(c3ccc4c(ccc5ccc6nc(-c7ccccc7)c(-c7ccccc7)nc6c54)c3)c3ccc4c(c3)sc3ccccc34)c2)cc1 ; c1ccc(-c2cccc(-c3ccccc3N(c3ccc4c(ccc5ccc6nc(-c7ccccc7)c(-c7ccccc7)nc6c54)c3)c3ccc4oc5ccccc5c4c3)c2)cc1]

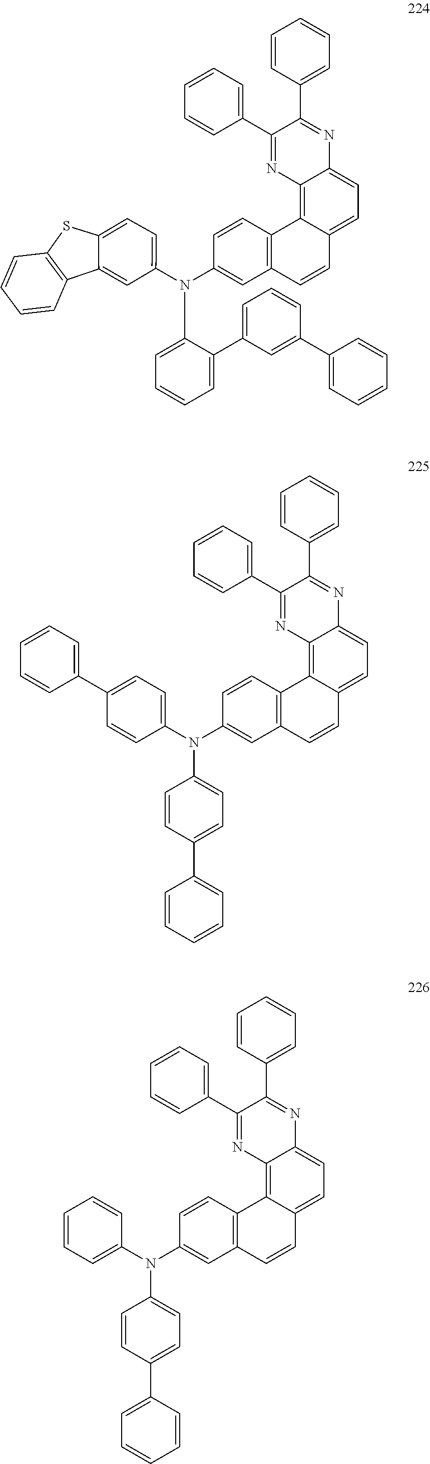 [SMILES: c1ccc(-c2cccc(-c3ccccc3N(c3ccc4c(ccc5ccc6nc(-c7ccccc7)c(-c7ccccc7)nc6c54)c3)c3ccc4sc5ccccc5c4c3)c2)cc1 ; c1ccc(-c2ccc(N(c3ccc(-c4ccccc4)cc3)c3ccc4c(ccc5ccc6nc(-c7ccccc7)c(-c7ccccc7)nc6c54)c3)cc2)cc1 ; c1ccc(-c2ccc(N(c3ccccc3)c3ccc4c(ccc5ccc6nc(-c7ccccc7)c(-c7ccccc7)nc6c54)c3)cc2)cc1]

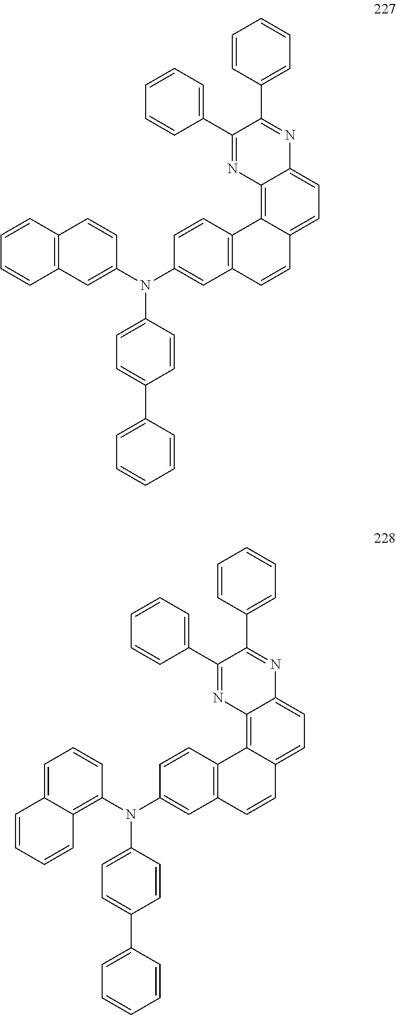 [SMILES: c1ccc(-c2ccc(N(c3ccc4ccccc4c3)c3ccc4c(ccc5ccc6nc(-c7ccccc7)c(-c7ccccc7)nc6c54)c3)cc2)cc1 ; c1ccc(-c2ccc(N(c3ccc4c(ccc5ccc6nc(-c7ccccc7)c(-c7ccccc7)nc6c54)c3)c3cccc4ccccc34)cc2)cc1]

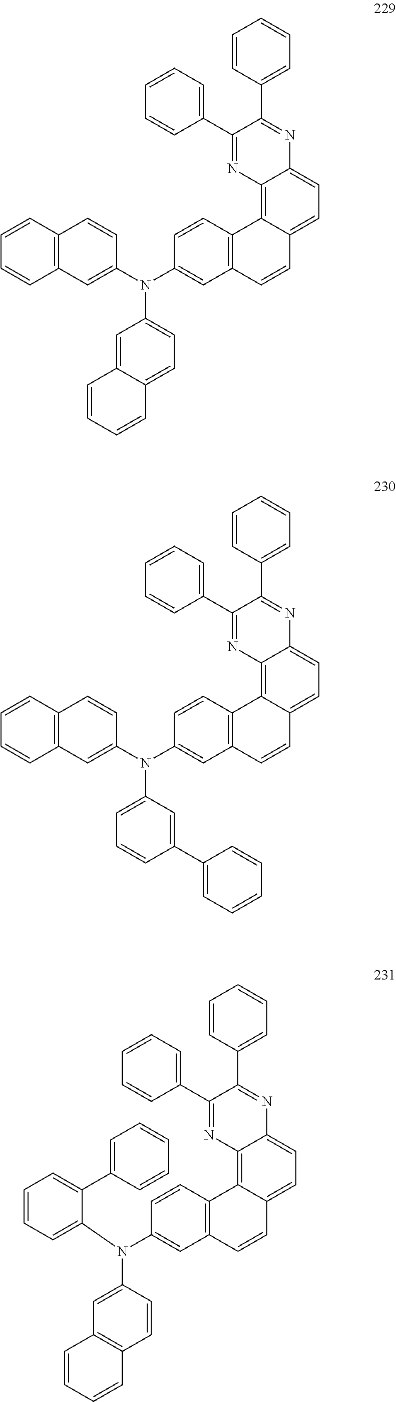 [SMILES: c1ccc(-c2nc3ccc4ccc5cc(N(c6ccc7ccccc7c6)c6ccc7ccccc7c6)ccc5c4c3nc2-c2ccccc2)cc1 ; c1ccc(-c2cccc(N(c3ccc4ccccc4c3)c3ccc4c(ccc5ccc6nc(-c7ccccc7)c(-c7ccccc7)nc6c54)c3)c2)cc1 ; c1ccc(-c2ccccc2N(c2ccc3ccccc3c2)c2ccc3c(ccc4ccc5nc(-c6ccccc6)c(-c6ccccc6)nc5c43)c2)cc1]

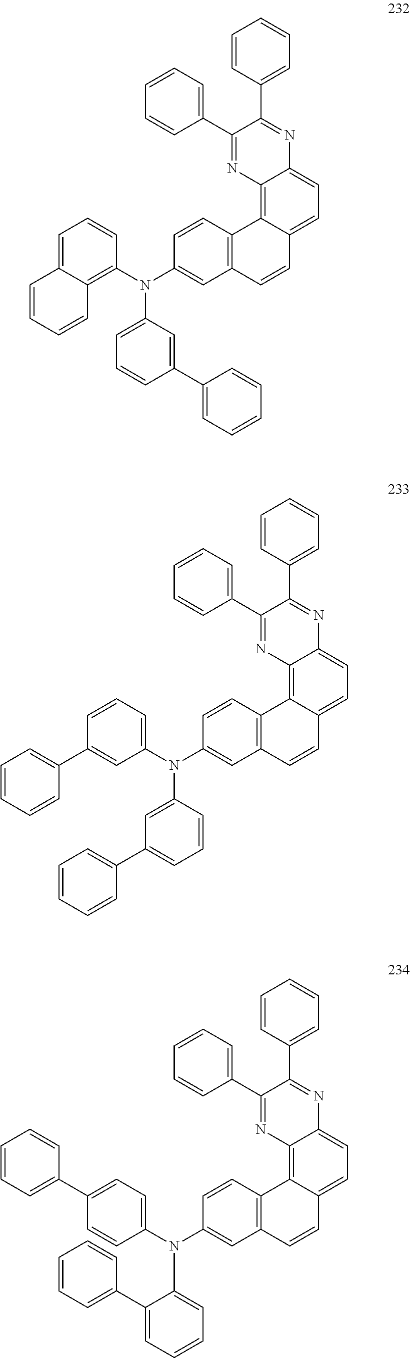 [SMILES: c1ccc(-c2cccc(N(c3ccc4c(ccc5ccc6nc(-c7ccccc7)c(-c7ccccc7)nc6c54)c3)c3cccc4ccccc34)c2)cc1 ; c1ccc(-c2cccc(N(c3cccc(-c4ccccc4)c3)c3ccc4c(ccc5ccc6nc(-c7ccccc7)c(-c7ccccc7)nc6c54)c3)c2)cc1 ; c1ccc(-c2ccc(N(c3ccc4c(ccc5ccc6nc(-c7ccccc7)c(-c7ccccc7)nc6c54)c3)c3ccccc3-c3ccccc3)cc2)cc1]

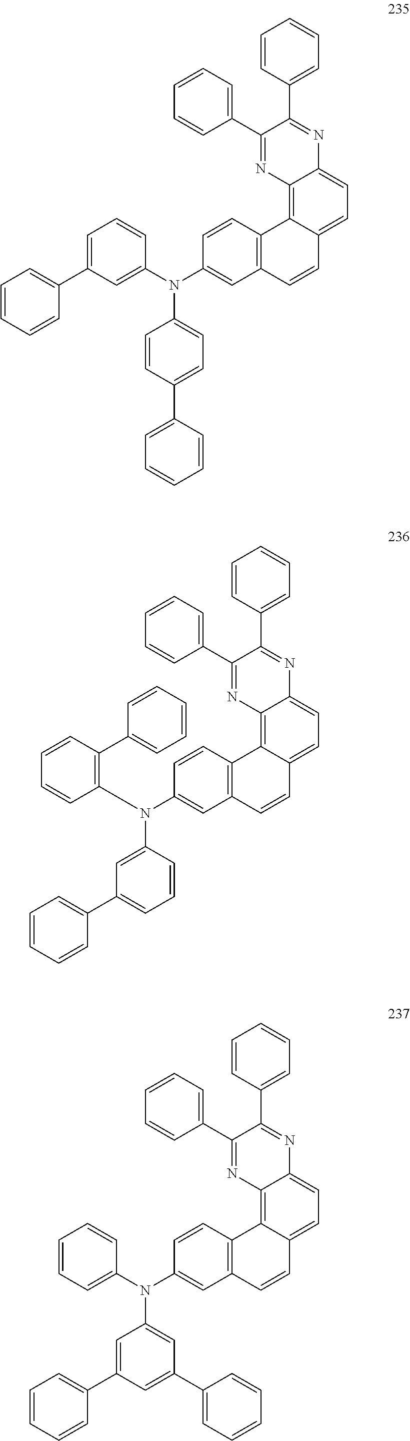 [SMILES: c1ccc(-c2ccc(N(c3cccc(-c4ccccc4)c3)c3ccc4c(ccc5ccc6nc(-c7ccccc7)c(-c7ccccc7)nc6c54)c3)cc2)cc1 ; c1ccc(-c2cccc(N(c3ccc4c(ccc5ccc6nc(-c7ccccc7)c(-c7ccccc7)nc6c54)c3)c3ccccc3-c3ccccc3)c2)cc1 ; c1ccc(-c2cc(-c3ccccc3)cc(N(c3ccccc3)c3ccc4c(ccc5ccc6nc(-c7ccccc7)c(-c7ccccc7)nc6c54)c3)c2)cc1]

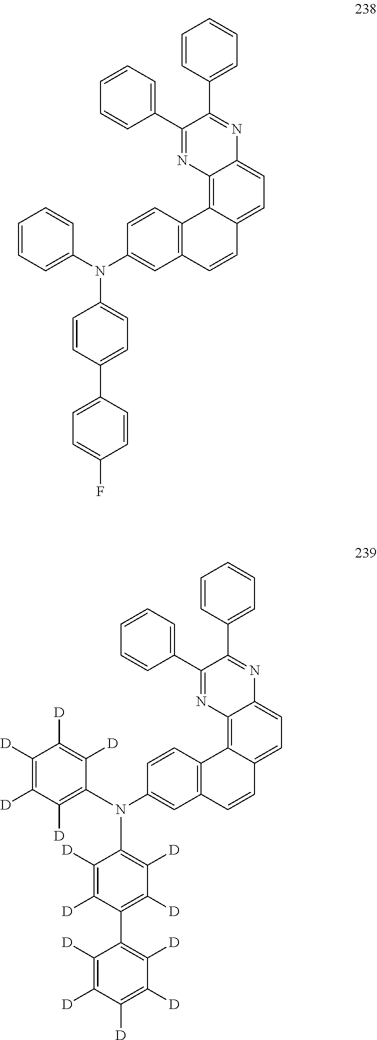 [SMILES: Fc1ccc(-c2ccc(N(c3ccccc3)c3ccc4c(ccc5ccc6nc(-c7ccccc7)c(-c7ccccc7)nc6c54)c3)cc2)cc1 ; [2H]c1c([2H])c([2H])c(-c2c([2H])c([2H])c(N(c3ccc4c(ccc5ccc6nc(-c7ccccc7)c(-c7ccccc7)nc6c54)c3)c3c([2H])c([2H])c([2H])c([2H])c3[2H])c([2H])c2[2H])c([2H])c1[2H]]

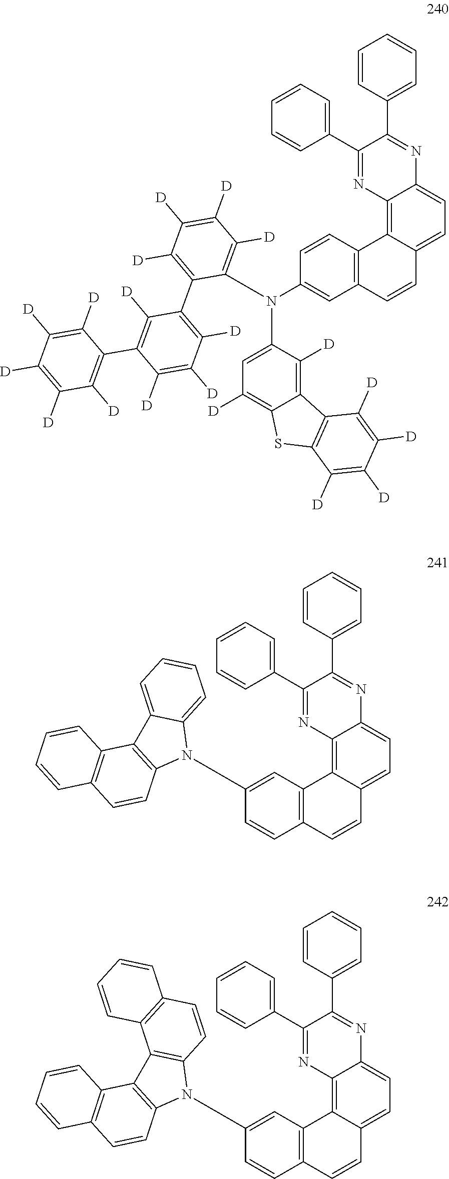 [SMILES: [2H]c1c([2H])c([2H])c(-c2c([2H])c([2H])c([2H])c(-c3c([2H])c([2H])c([2H])c([2H])c3N(c3ccc4c(ccc5ccc6nc(-c7ccccc7)c(-c7ccccc7)nc6c54)c3)c3cc([2H])c4sc5c([2H])c([2H])c([2H])c([2H])c5c4c3[2H])c2[2H])c([2H])c1[2H] ; c1ccc(-c2nc3ccc4ccc5ccc(-n6c7ccccc7c7c8ccccc8ccc76)cc5c4c3nc2-c2ccccc2)cc1 ; c1ccc(-c2nc3ccc4ccc5ccc(-n6c7ccc8ccccc8c7c7c8ccccc8ccc76)cc5c4c3nc2-c2ccccc2)cc1]

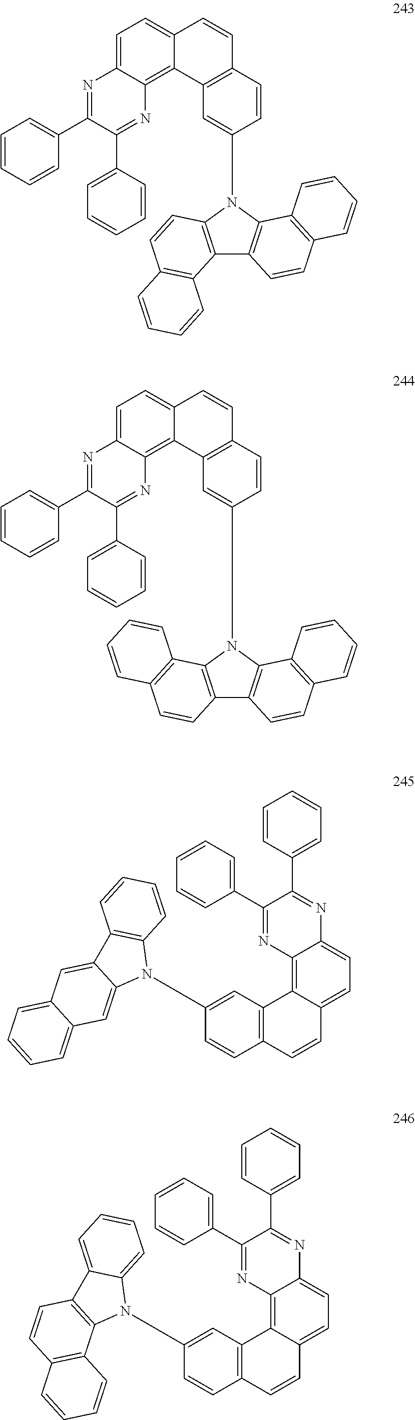 [SMILES: c1ccc(-c2nc3ccc4ccc5ccc(-n6c7ccc8ccccc8c7c7ccc8ccccc8c76)cc5c4c3nc2-c2ccccc2)cc1 ; c1ccc(-c2nc3ccc4ccc5ccc(-n6c7c8ccccc8ccc7c7ccc8ccccc8c76)cc5c4c3nc2-c2ccccc2)cc1 ; c1ccc(-c2nc3ccc4ccc5ccc(-n6c7ccccc7c7cc8ccccc8cc76)cc5c4c3nc2-c2ccccc2)cc1 ; c1ccc(-c2nc3ccc4ccc5ccc(-n6c7ccccc7c7ccc8ccccc8c76)cc5c4c3nc2-c2ccccc2)cc1]

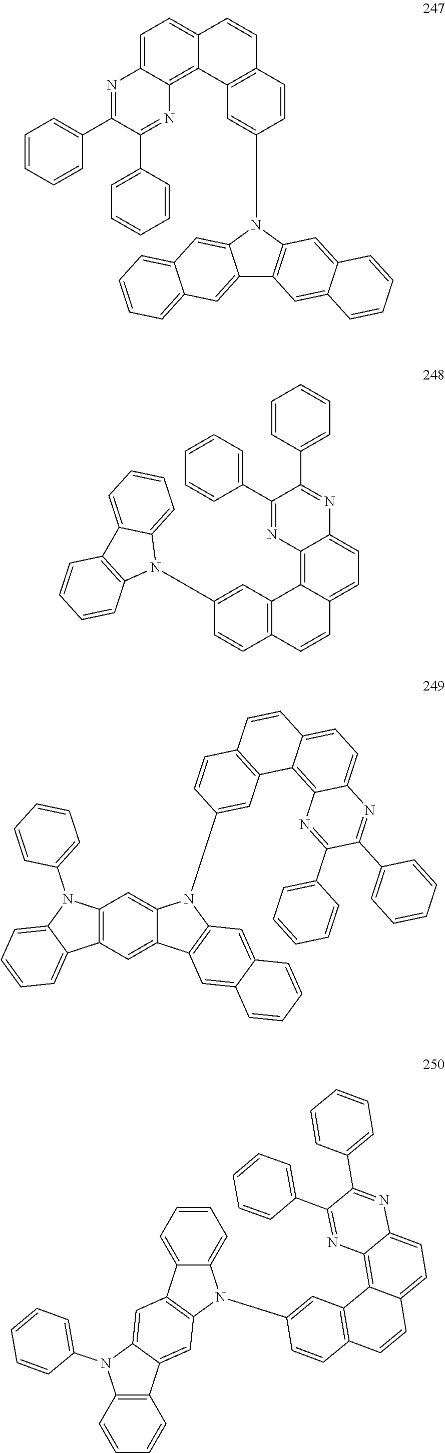 [SMILES: c1ccc(-c2nc3ccc4ccc5ccc(-n6c7cc8ccccc8cc7c7cc8ccccc8cc76)cc5c4c3nc2-c2ccccc2)cc1 ; c1ccc(-c2nc3ccc4ccc5ccc(-n6c7ccccc7c7ccccc76)cc5c4c3nc2-c2ccccc2)cc1 ; c1ccc(-c2nc3ccc4ccc5ccc(-n6c7cc8ccccc8cc7c7cc8c9ccccc9n(-c9ccccc9)c8cc76)cc5c4c3nc2-c2ccccc2)cc1 ; c1ccc(-c2nc3ccc4ccc5ccc(-n6c7ccccc7c7cc8c(cc76)c6ccccc6n8-c6ccccc6)cc5c4c3nc2-c2ccccc2)cc1]

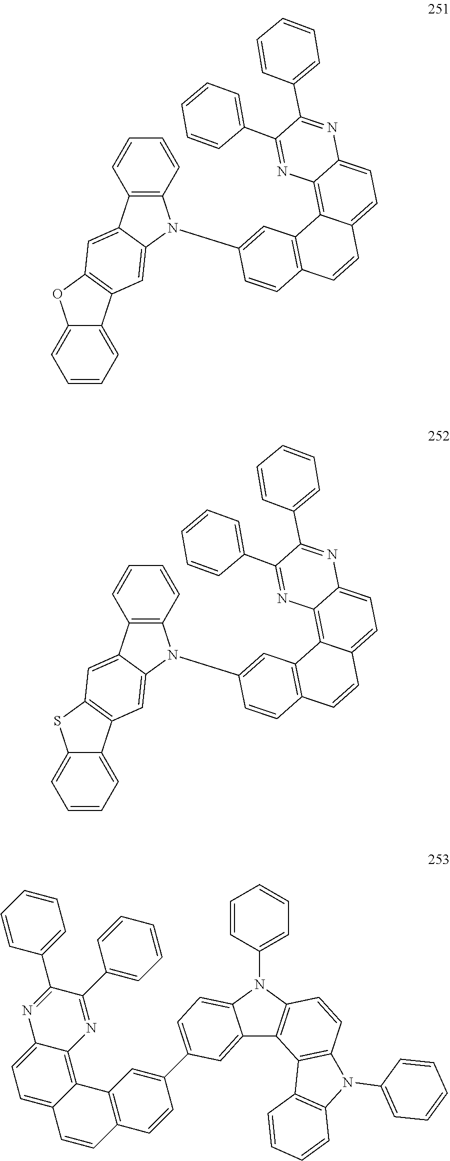 [SMILES: c1ccc(-c2nc3ccc4ccc5ccc(-n6c7ccccc7c7cc8oc9ccccc9c8cc76)cc5c4c3nc2-c2ccccc2)cc1 ; c1ccc(-c2nc3ccc4ccc5ccc(-n6c7ccccc7c7cc8sc9ccccc9c8cc76)cc5c4c3nc2-c2ccccc2)cc1 ; c1ccc(-c2nc3ccc4ccc5ccc(-c6ccc7c(c6)c6c8c9ccccc9n(-c9ccccc9)c8ccc6n7-c6ccccc6)cc5c4c3nc2-c2ccccc2)cc1]

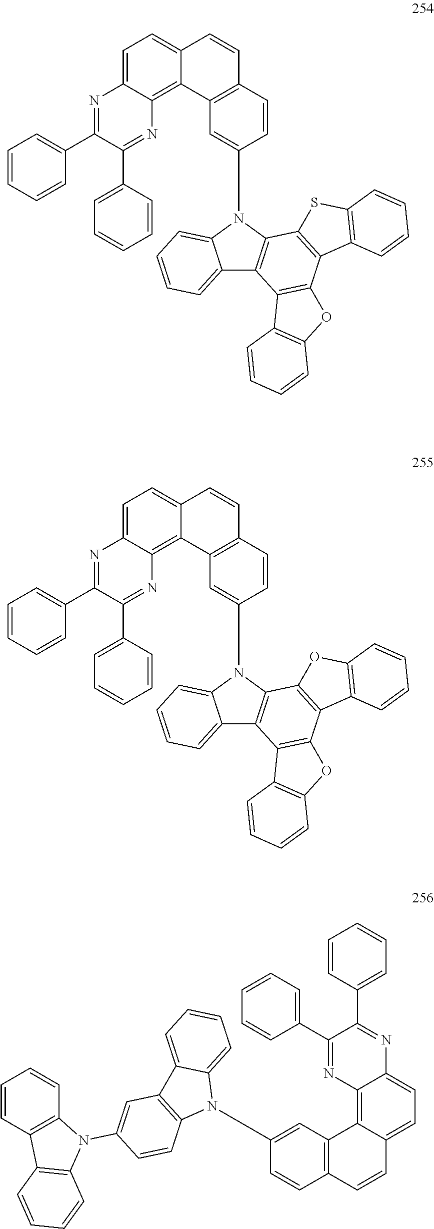 [SMILES: c1ccc(-c2nc3ccc4ccc5ccc(-n6c7ccccc7c7c8c9ccccc9oc8c8c9ccccc9sc8c76)cc5c4c3nc2-c2ccccc2)cc1 ; c1ccc(-c2nc3ccc4ccc5ccc(-n6c7ccccc7c7c8c9ccccc9oc8c8c9ccccc9oc8c76)cc5c4c3nc2-c2ccccc2)cc1 ; c1ccc(-c2nc3ccc4ccc5ccc(-n6c7ccccc7c7cc(-n8c9ccccc9c9ccccc98)ccc76)cc5c4c3nc2-c2ccccc2)cc1]

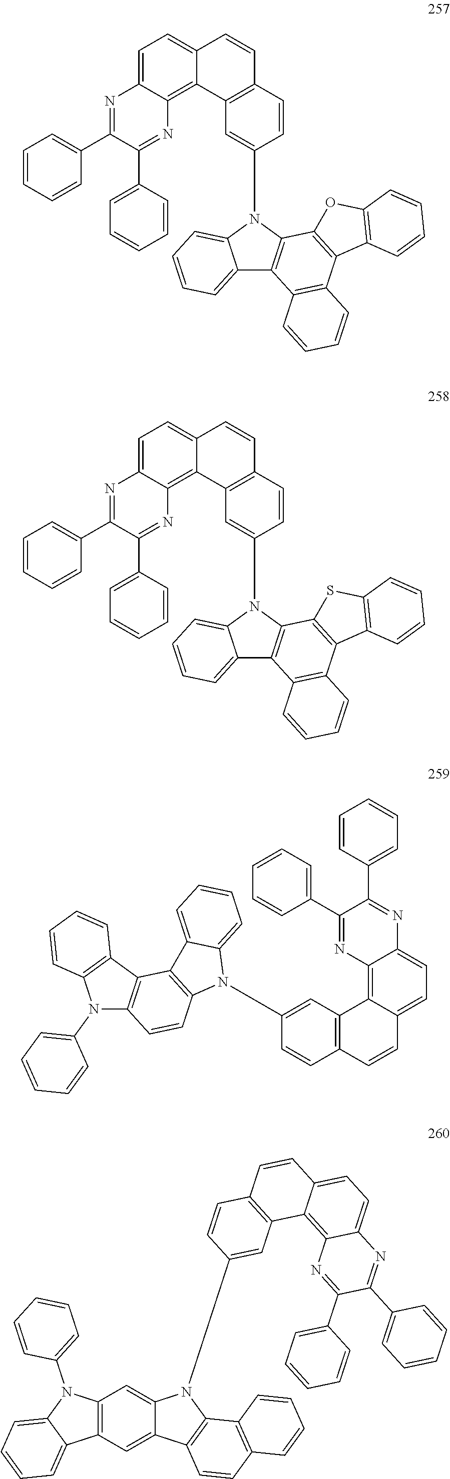 [SMILES: c1ccc(-c2nc3ccc4ccc5ccc(-n6c7ccccc7c7c8ccccc8c8c9ccccc9oc8c76)cc5c4c3nc2-c2ccccc2)cc1 ; c1ccc(-c2nc3ccc4ccc5ccc(-n6c7ccccc7c7c8ccccc8c8c9ccccc9sc8c76)cc5c4c3nc2-c2ccccc2)cc1 ; c1ccc(-c2nc3ccc4ccc5ccc(-n6c7ccccc7c7c8c9ccccc9n(-c9ccccc9)c8ccc76)cc5c4c3nc2-c2ccccc2)cc1 ; c1ccc(-c2nc3ccc4ccc5ccc(-n6c7cc8c(cc7c7ccc9ccccc9c76)c6ccccc6n8-c6ccccc6)cc5c4c3nc2-c2ccccc2)cc1]

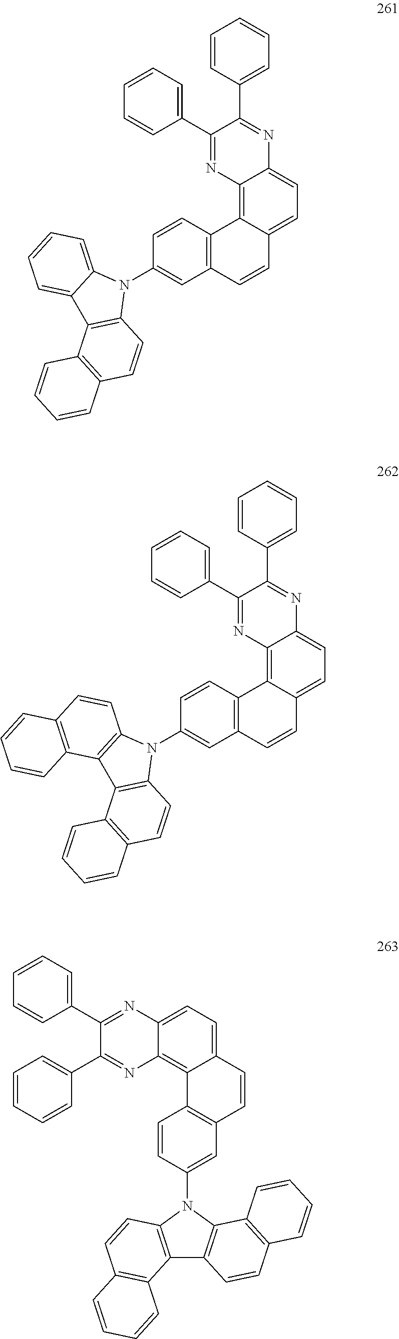 [SMILES: c1ccc(-c2nc3ccc4ccc5cc(-n6c7ccccc7c7c8ccccc8ccc76)ccc5c4c3nc2-c2ccccc2)cc1 ; c1ccc(-c2nc3ccc4ccc5cc(-n6c7ccc8ccccc8c7c7c8ccccc8ccc76)ccc5c4c3nc2-c2ccccc2)cc1 ; c1ccc(-c2nc3ccc4ccc5cc(-n6c7ccc8ccccc8c7c7ccc8ccccc8c76)ccc5c4c3nc2-c2ccccc2)cc1]

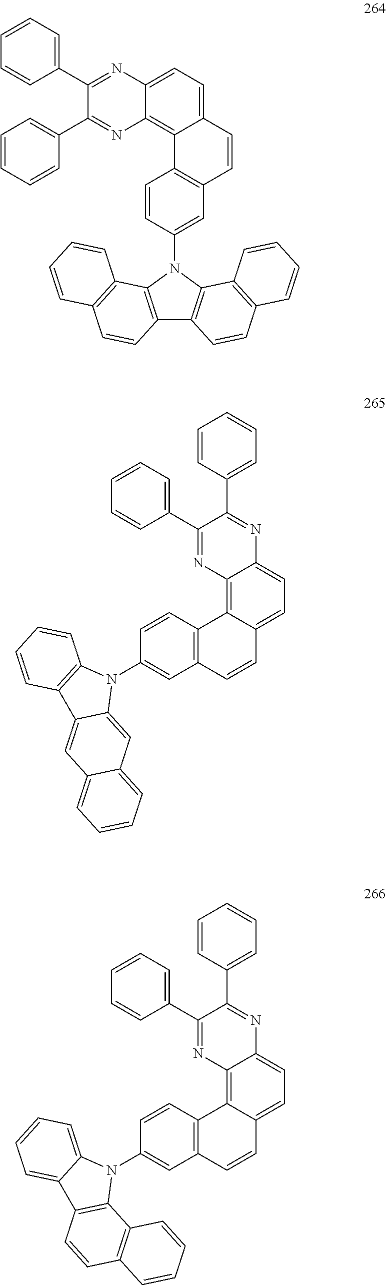 [SMILES: c1ccc(-c2nc3ccc4ccc5cc(-n6c7c8ccccc8ccc7c7ccc8ccccc8c76)ccc5c4c3nc2-c2ccccc2)cc1 ; c1ccc(-c2nc3ccc4ccc5cc(-n6c7ccccc7c7cc8ccccc8cc76)ccc5c4c3nc2-c2ccccc2)cc1 ; c1ccc(-c2nc3ccc4ccc5cc(-n6c7ccccc7c7ccc8ccccc8c76)ccc5c4c3nc2-c2ccccc2)cc1]

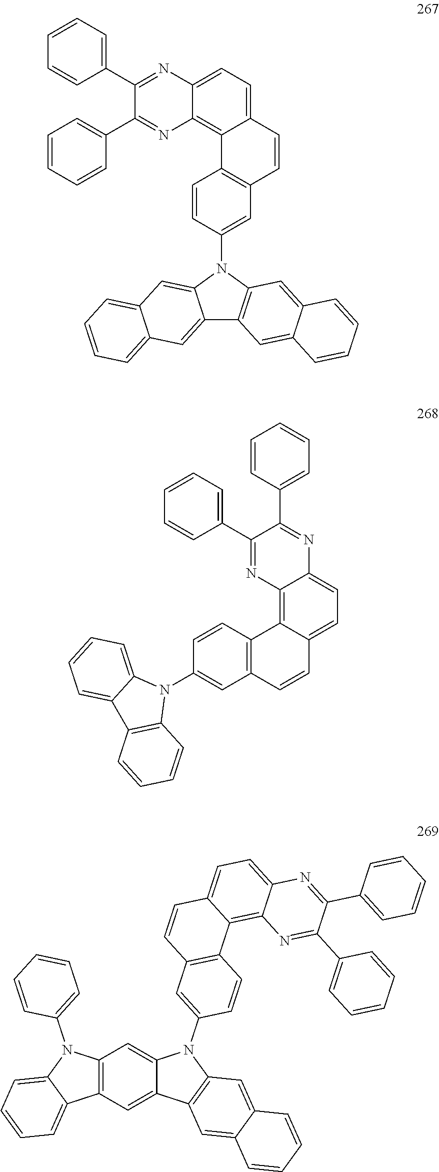 [SMILES: c1ccc(-c2nc3ccc4ccc5cc(-n6c7cc8ccccc8cc7c7cc8ccccc8cc76)ccc5c4c3nc2-c2ccccc2)cc1 ; c1ccc(-c2nc3ccc4ccc5cc(-n6c7ccccc7c7ccccc76)ccc5c4c3nc2-c2ccccc2)cc1 ; c1ccc(-c2nc3ccc4ccc5cc(-n6c7cc8ccccc8cc7c7cc8c9ccccc9n(-c9ccccc9)c8cc76)ccc5c4c3nc2-c2ccccc2)cc1]

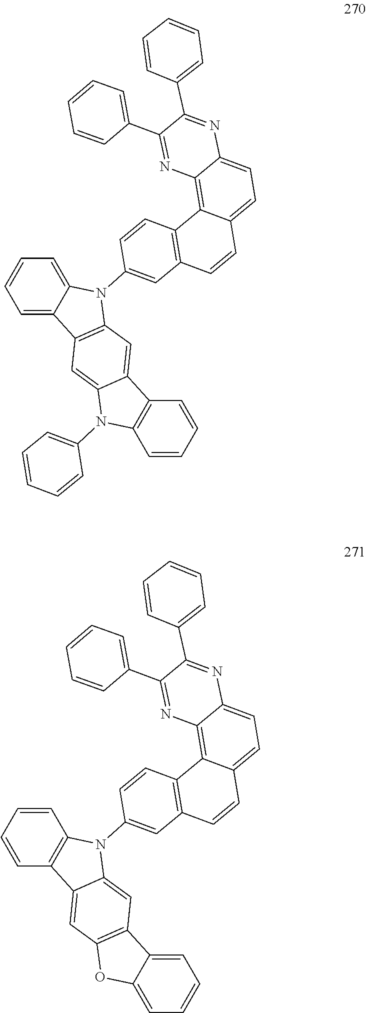 [SMILES: c1ccc(-c2nc3ccc4ccc5cc(-n6c7ccccc7c7cc8c(cc76)c6ccccc6n8-c6ccccc6)ccc5c4c3nc2-c2ccccc2)cc1 ; c1ccc(-c2nc3ccc4ccc5cc(-n6c7ccccc7c7cc8oc9ccccc9c8cc76)ccc5c4c3nc2-c2ccccc2)cc1]

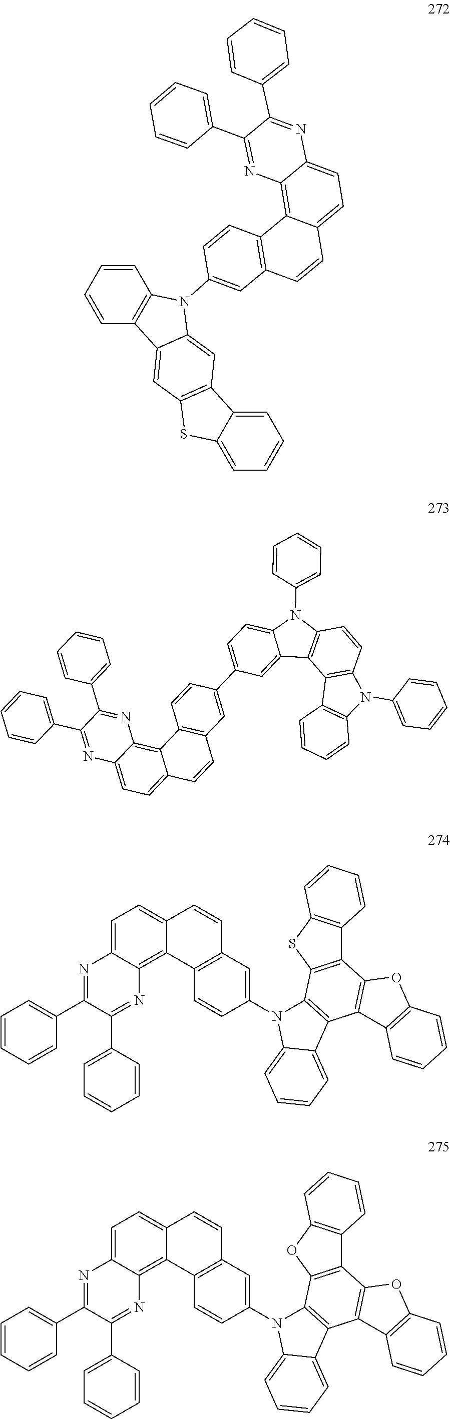 [SMILES: c1ccc(-c2nc3ccc4ccc5cc(-n6c7ccccc7c7cc8sc9ccccc9c8cc76)ccc5c4c3nc2-c2ccccc2)cc1 ; c1ccc(-c2nc3ccc4ccc5cc(-c6ccc7c(c6)c6c8c9ccccc9n(-c9ccccc9)c8ccc6n7-c6ccccc6)ccc5c4c3nc2-c2ccccc2)cc1 ; c1ccc(-c2nc3ccc4ccc5cc(-n6c7ccccc7c7c8c9ccccc9oc8c8c9ccccc9sc8c76)ccc5c4c3nc2-c2ccccc2)cc1 ; c1ccc(-c2nc3ccc4ccc5cc(-n6c7ccccc7c7c8c9ccccc9oc8c8c9ccccc9oc8c76)ccc5c4c3nc2-c2ccccc2)cc1]

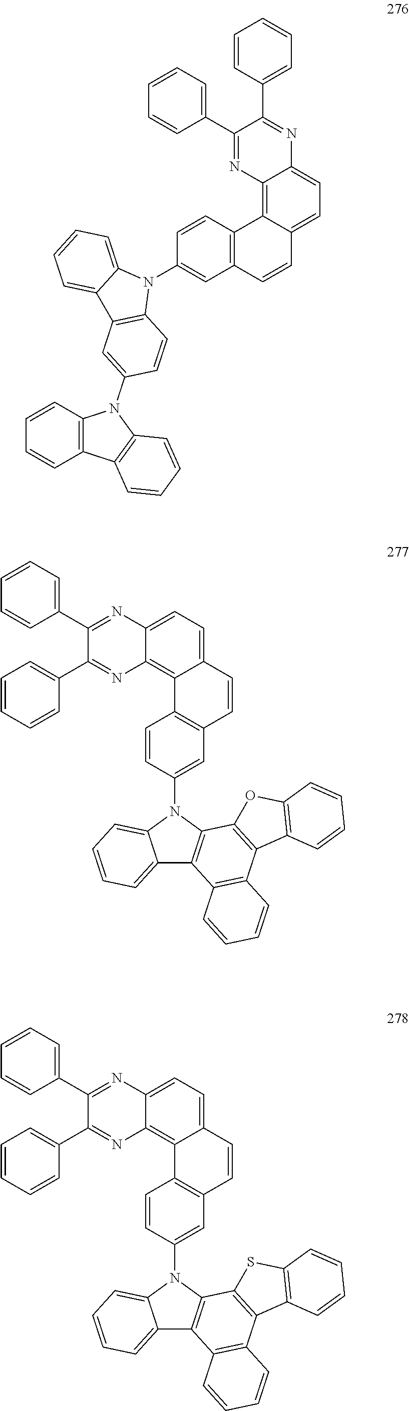 [SMILES: c1ccc(-c2nc3ccc4ccc5cc(-n6c7ccccc7c7cc(-n8c9ccccc9c9ccccc98)ccc76)ccc5c4c3nc2-c2ccccc2)cc1 ; c1ccc(-c2nc3ccc4ccc5cc(-n6c7ccccc7c7c8ccccc8c8c9ccccc9oc8c76)ccc5c4c3nc2-c2ccccc2)cc1 ; c1ccc(-c2nc3ccc4ccc5cc(-n6c7ccccc7c7c8ccccc8c8c9ccccc9sc8c76)ccc5c4c3nc2-c2ccccc2)cc1]

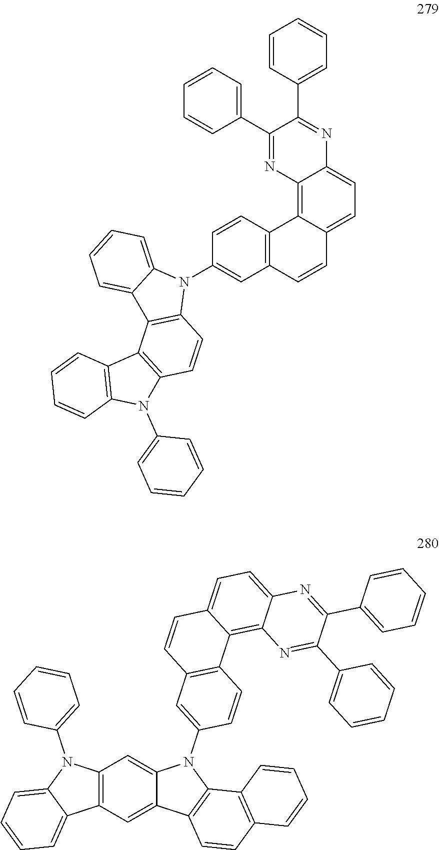 [SMILES: c1ccc(-c2nc3ccc4ccc5cc(-n6c7ccccc7c7c8c9ccccc9n(-c9ccccc9)c8ccc76)ccc5c4c3nc2-c2ccccc2)cc1 ; c1ccc(-c2nc3ccc4ccc5cc(-n6c7cc8c(cc7c7ccc9ccccc9c76)c6ccccc6n8-c6ccccc6)ccc5c4c3nc2-c2ccccc2)cc1]

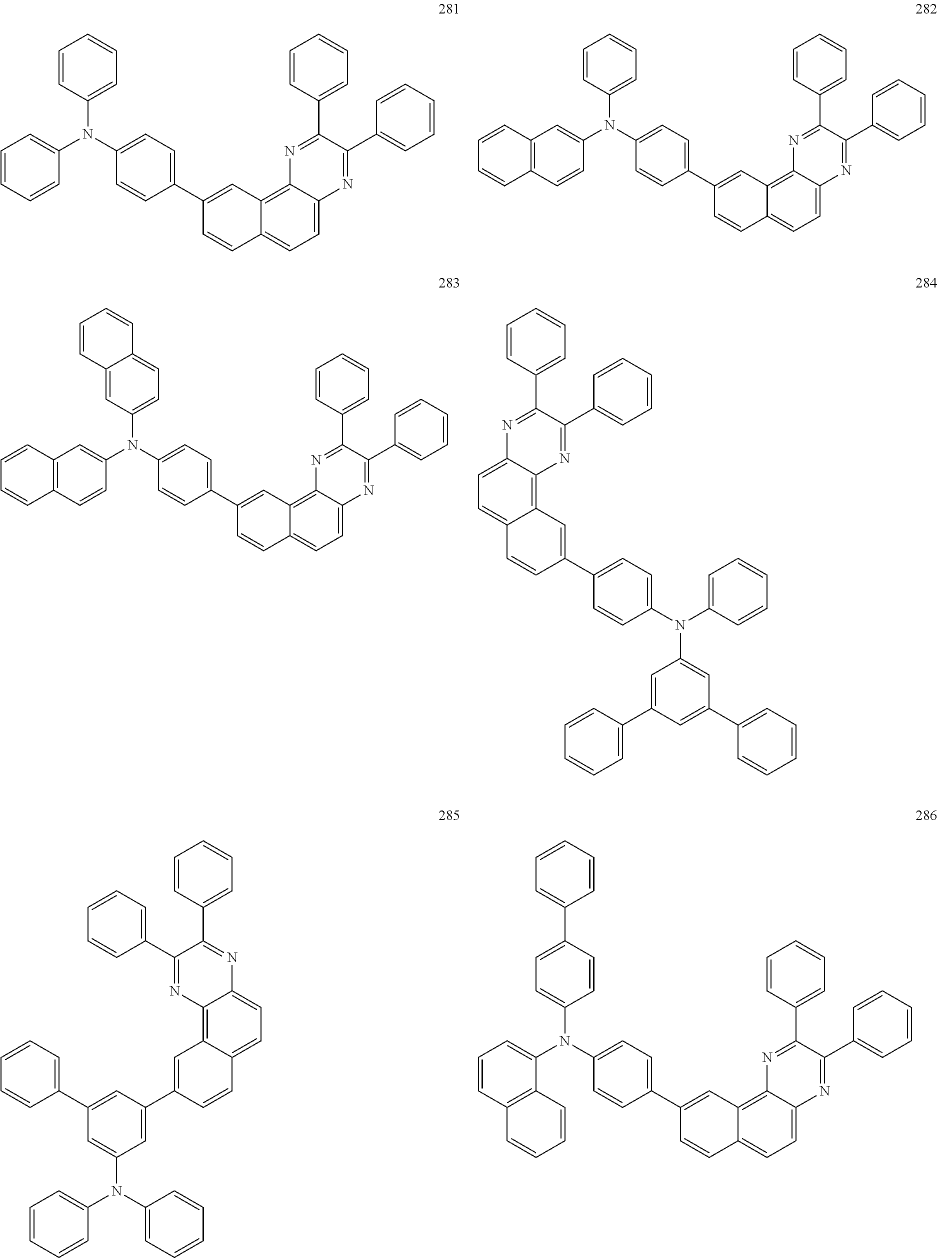

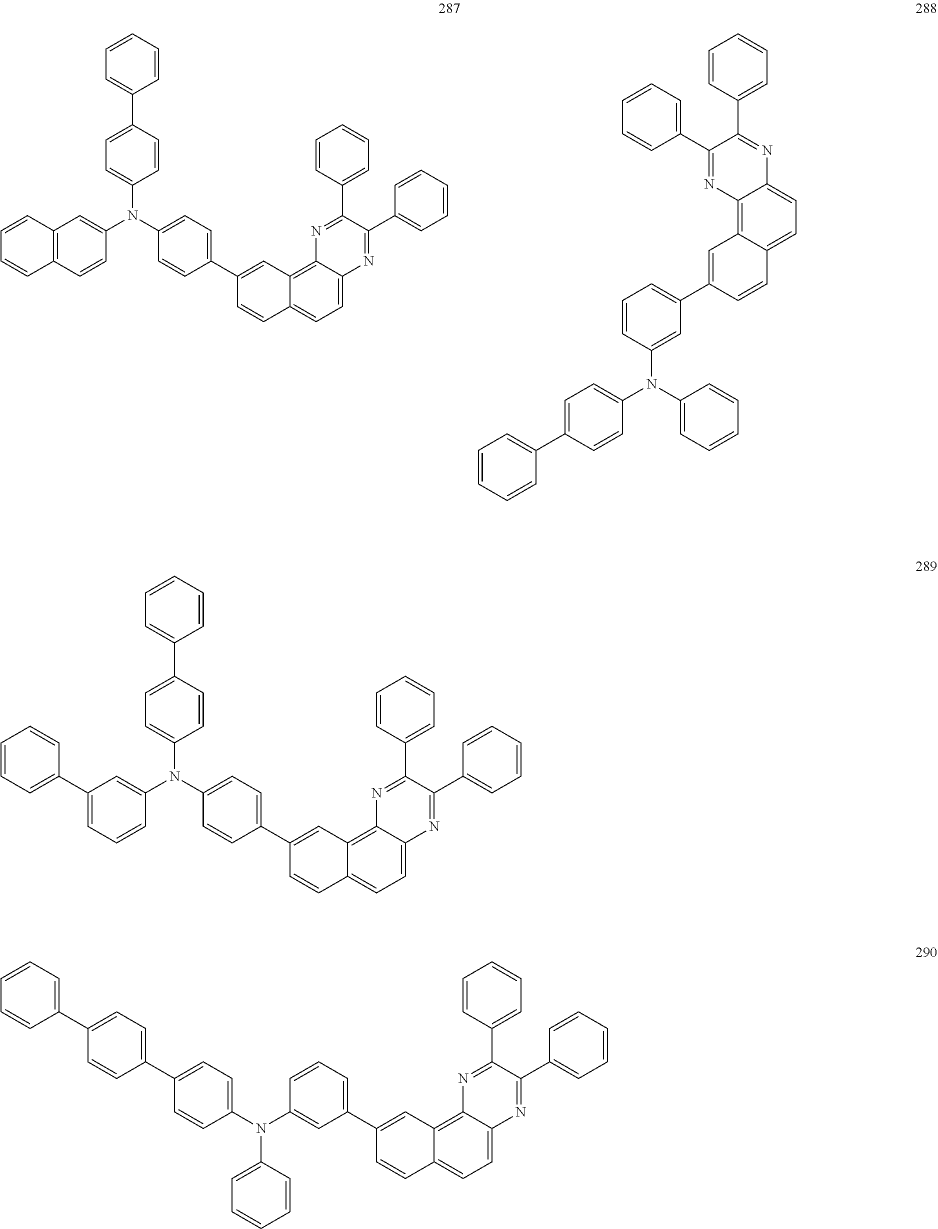 [SMILES: c1ccc(-c2ccc(N(c3ccc(-c4ccc5ccc6nc(-c7ccccc7)c(-c7ccccc7)nc6c5c4)cc3)c3ccc4ccccc4c3)cc2)cc1 ; c1ccc(-c2ccc(N(c3ccccc3)c3cccc(-c4ccc5ccc6nc(-c7ccccc7)c(-c7ccccc7)nc6c5c4)c3)cc2)cc1 ; c1ccc(-c2ccc(N(c3ccc(-c4ccc5ccc6nc(-c7ccccc7)c(-c7ccccc7)nc6c5c4)cc3)c3cccc(-c4ccccc4)c3)cc2)cc1 ; c1ccc(-c2ccc(-c3ccc(N(c4ccccc4)c4cccc(-c5ccc6ccc7nc(-c8ccccc8)c(-c8ccccc8)nc7c6c5)c4)cc3)cc2)cc1]

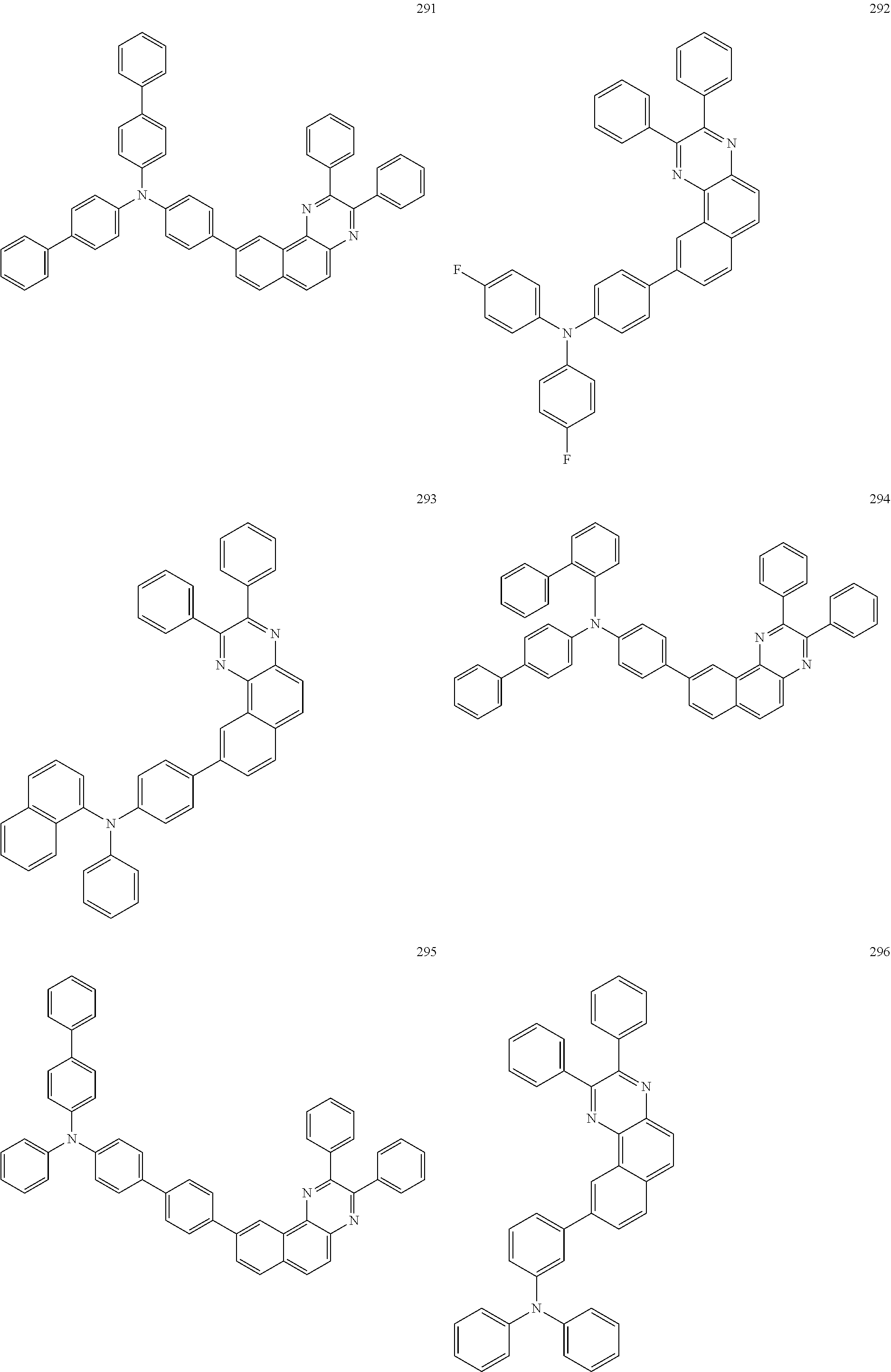

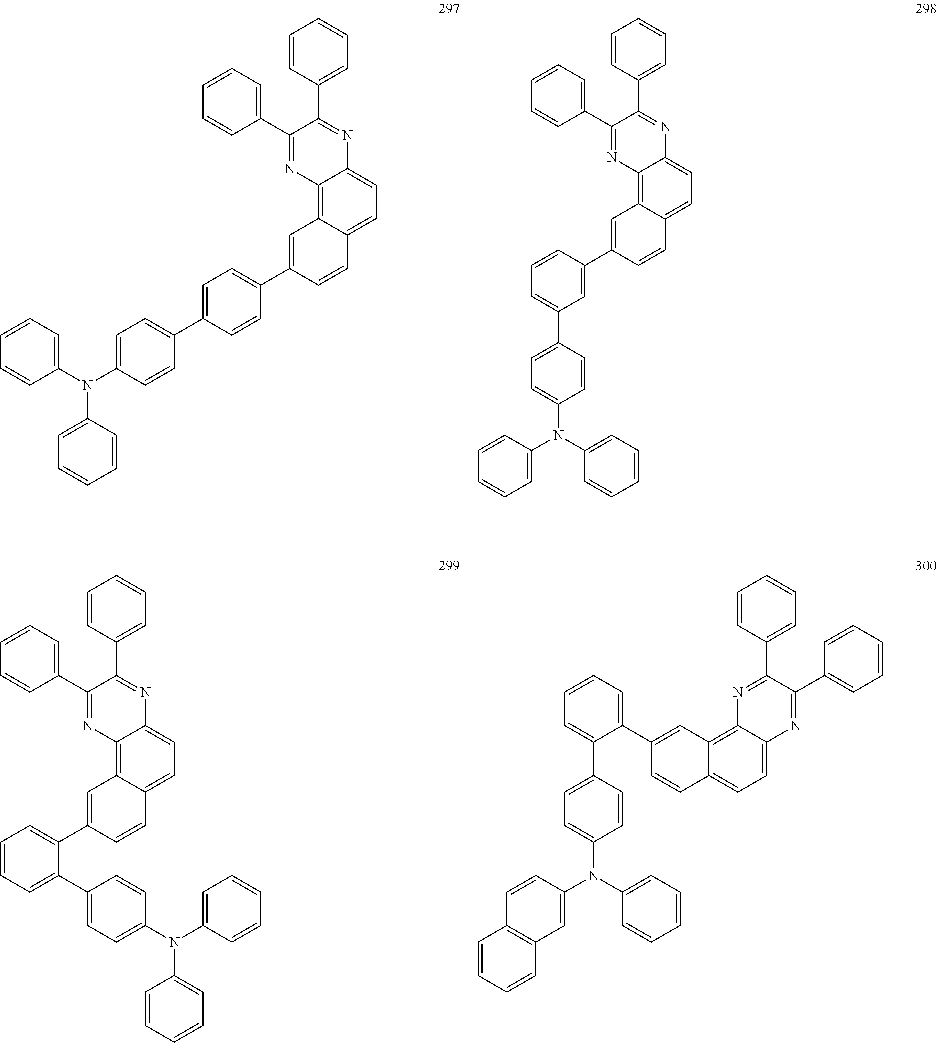 [SMILES: c1ccc(-c2nc3ccc4ccc(-c5ccc(-c6ccc(N(c7ccccc7)c7ccccc7)cc6)cc5)cc4c3nc2-c2ccccc2)cc1 ; c1ccc(-c2nc3ccc4ccc(-c5cccc(-c6ccc(N(c7ccccc7)c7ccccc7)cc6)c5)cc4c3nc2-c2ccccc2)cc1 ; c1ccc(-c2nc3ccc4ccc(-c5ccccc5-c5ccc(N(c6ccccc6)c6ccccc6)cc5)cc4c3nc2-c2ccccc2)cc1 ; c1ccc(-c2nc3ccc4ccc(-c5ccccc5-c5ccc(N(c6ccccc6)c6ccc7ccccc7c6)cc5)cc4c3nc2-c2ccccc2)cc1]

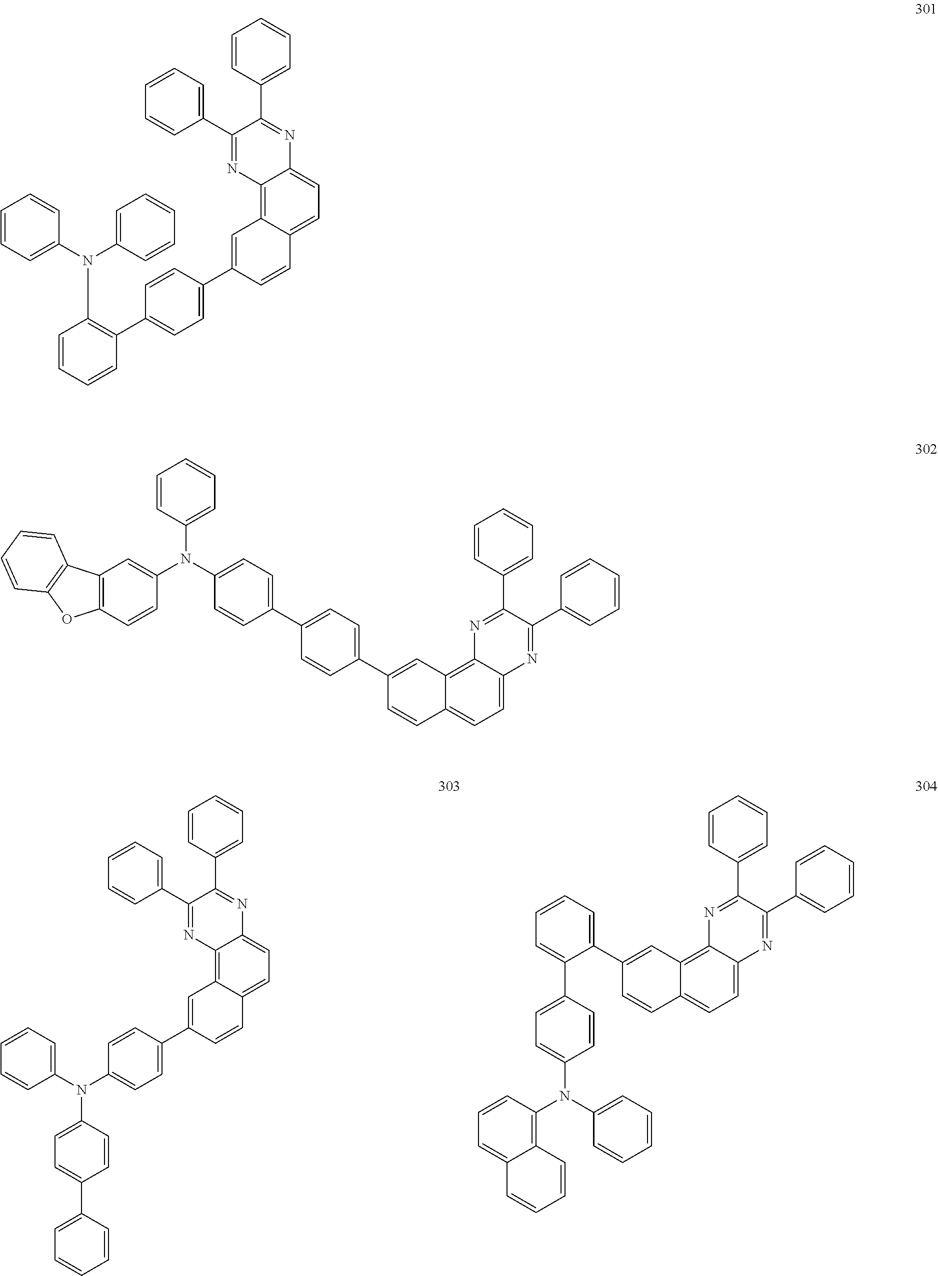 [SMILES: c1ccc(-c2nc3ccc4ccc(-c5ccc(-c6ccccc6N(c6ccccc6)c6ccccc6)cc5)cc4c3nc2-c2ccccc2)cc1 ; c1ccc(-c2nc3ccc4ccc(-c5ccc(-c6ccc(N(c7ccccc7)c7ccc8oc9ccccc9c8c7)cc6)cc5)cc4c3nc2-c2ccccc2)cc1 ; c1ccc(-c2ccc(N(c3ccccc3)c3ccc(-c4ccc5ccc6nc(-c7ccccc7)c(-c7ccccc7)nc6c5c4)cc3)cc2)cc1 ; c1ccc(-c2nc3ccc4ccc(-c5ccccc5-c5ccc(N(c6ccccc6)c6cccc7ccccc67)cc5)cc4c3nc2-c2ccccc2)cc1]

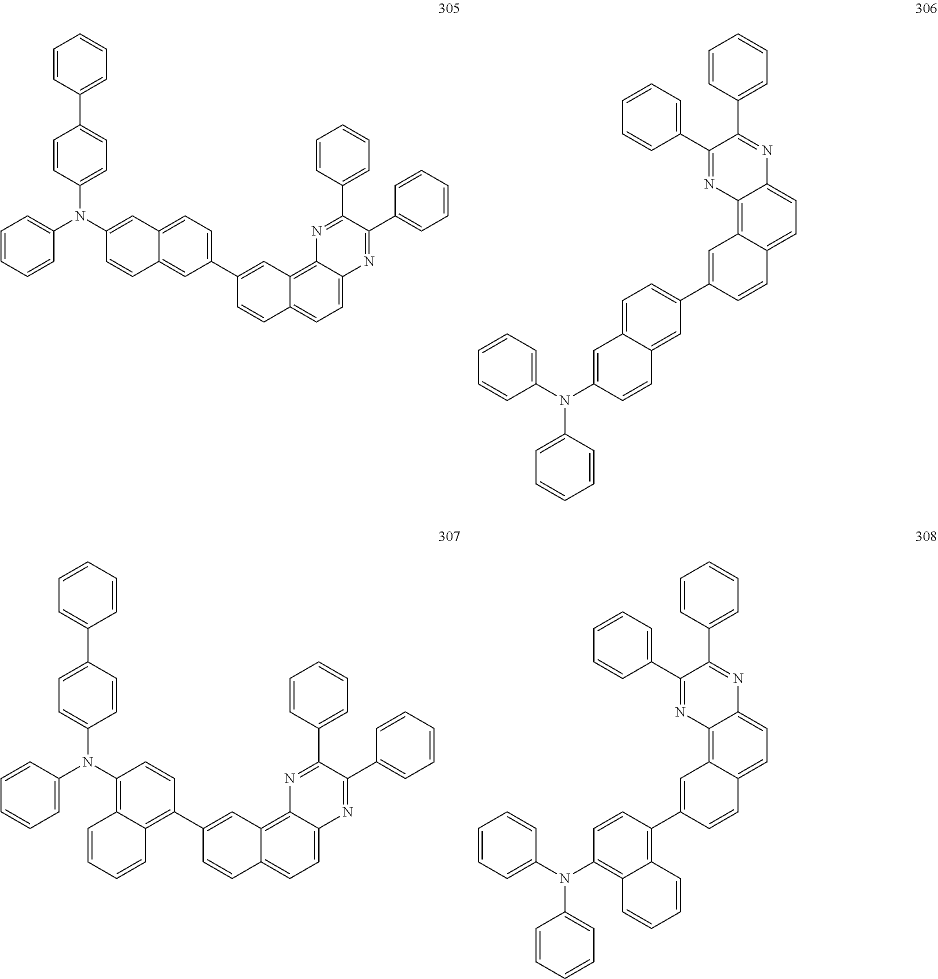 [SMILES: c1ccc(-c2ccc(N(c3ccccc3)c3ccc4cc(-c5ccc6ccc7nc(-c8ccccc8)c(-c8ccccc8)nc7c6c5)ccc4c3)cc2)cc1 ; c1ccc(-c2nc3ccc4ccc(-c5ccc6cc(N(c7ccccc7)c7ccccc7)ccc6c5)cc4c3nc2-c2ccccc2)cc1 ; c1ccc(-c2ccc(N(c3ccccc3)c3ccc(-c4ccc5ccc6nc(-c7ccccc7)c(-c7ccccc7)nc6c5c4)c4ccccc34)cc2)cc1 ; c1ccc(-c2nc3ccc4ccc(-c5ccc(N(c6ccccc6)c6ccccc6)c6ccccc56)cc4c3nc2-c2ccccc2)cc1]

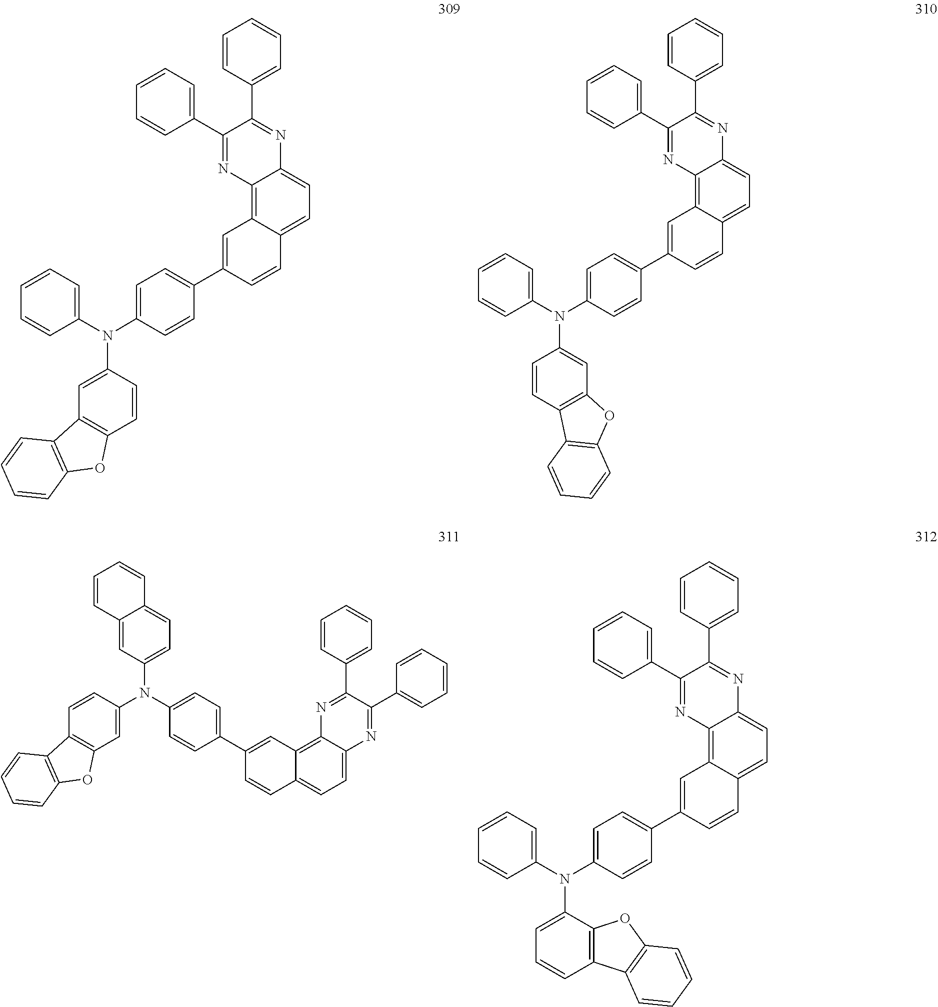 [SMILES: c1ccc(-c2nc3ccc4ccc(-c5ccc(N(c6ccccc6)c6ccc7oc8ccccc8c7c6)cc5)cc4c3nc2-c2ccccc2)cc1 ; c1ccc(-c2nc3ccc4ccc(-c5ccc(N(c6ccccc6)c6ccc7c(c6)oc6ccccc67)cc5)cc4c3nc2-c2ccccc2)cc1 ; c1ccc(-c2nc3ccc4ccc(-c5ccc(N(c6ccc7ccccc7c6)c6ccc7c(c6)oc6ccccc67)cc5)cc4c3nc2-c2ccccc2)cc1 ; c1ccc(-c2nc3ccc4ccc(-c5ccc(N(c6ccccc6)c6cccc7c6oc6ccccc67)cc5)cc4c3nc2-c2ccccc2)cc1]

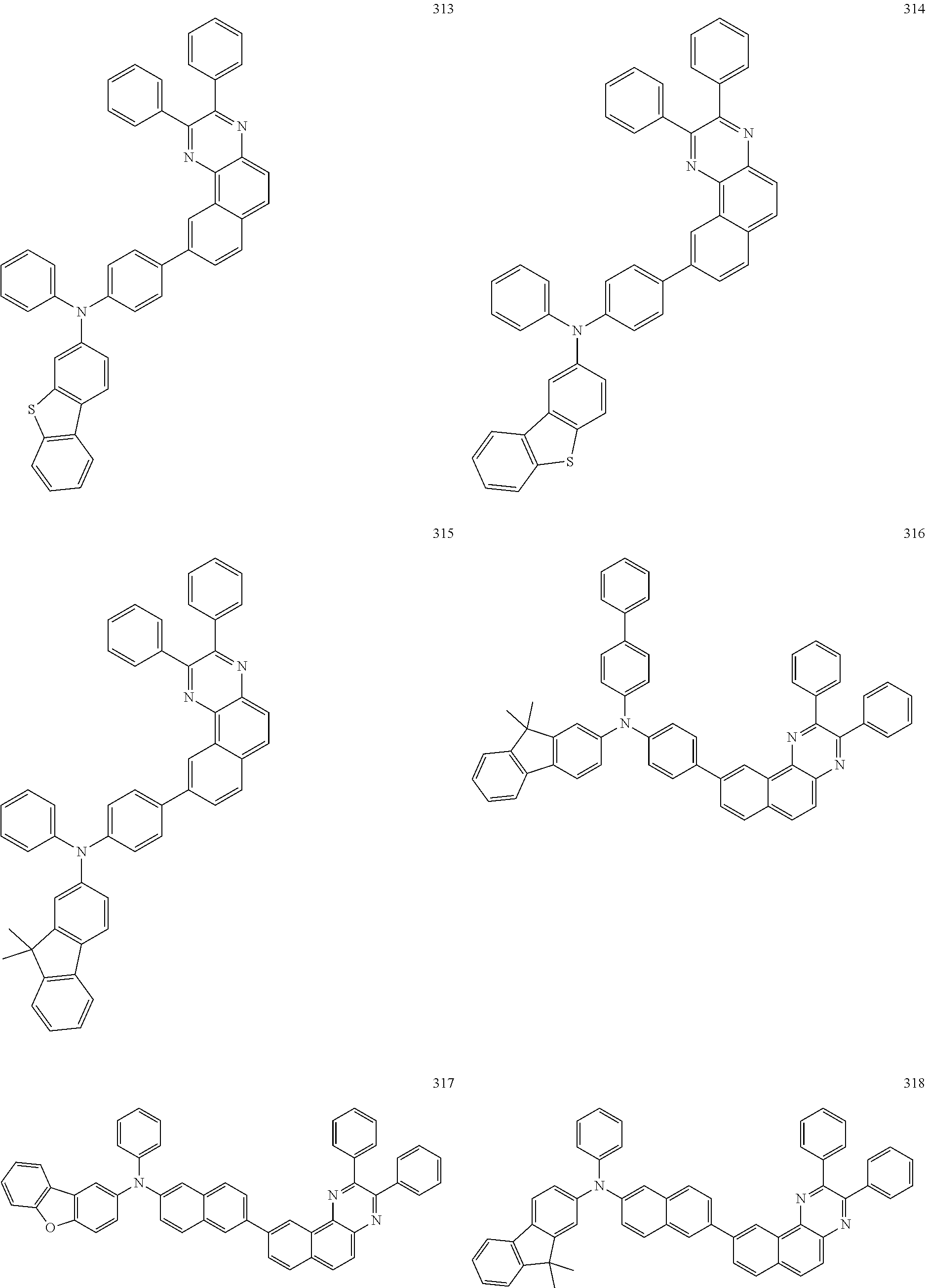

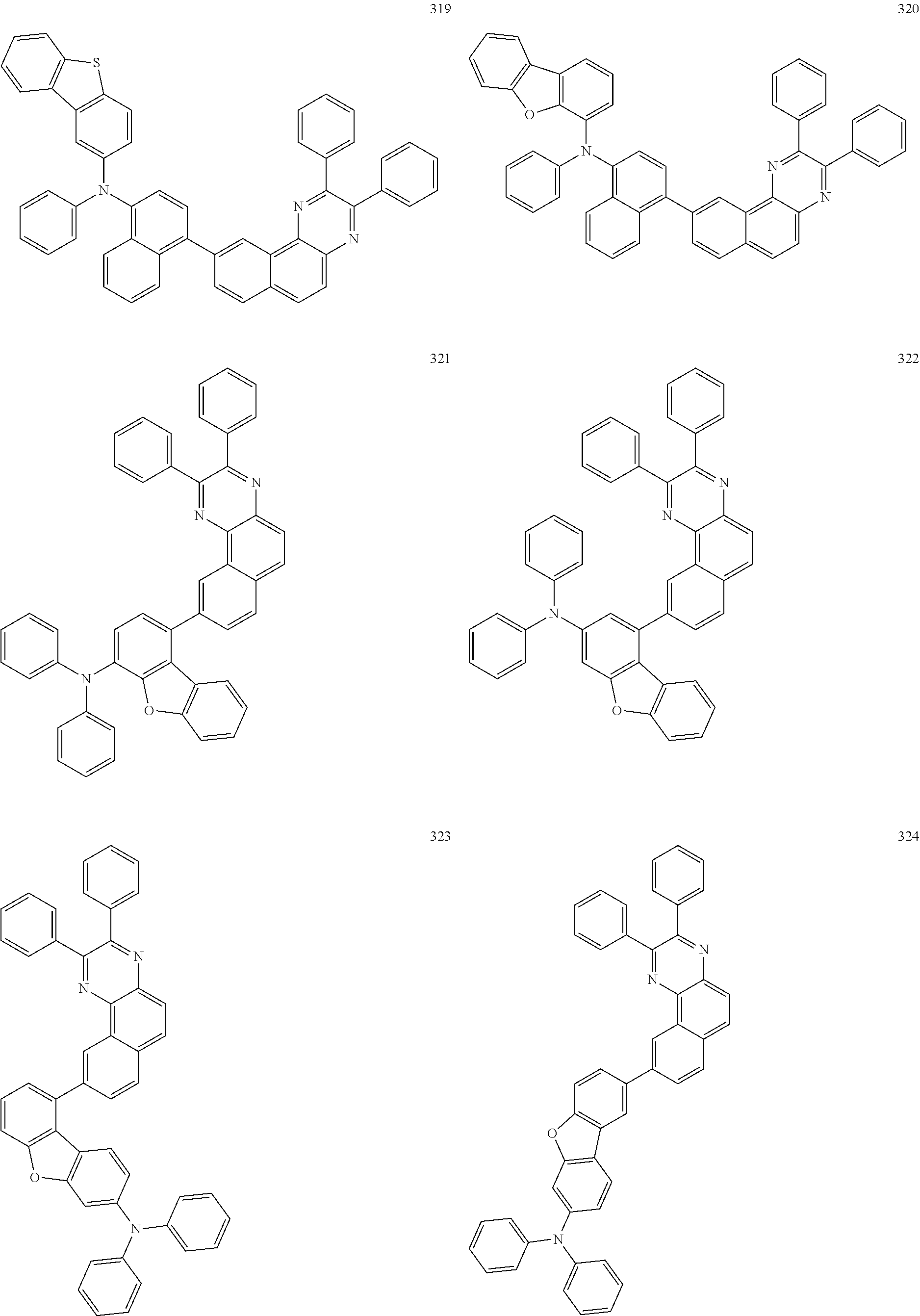

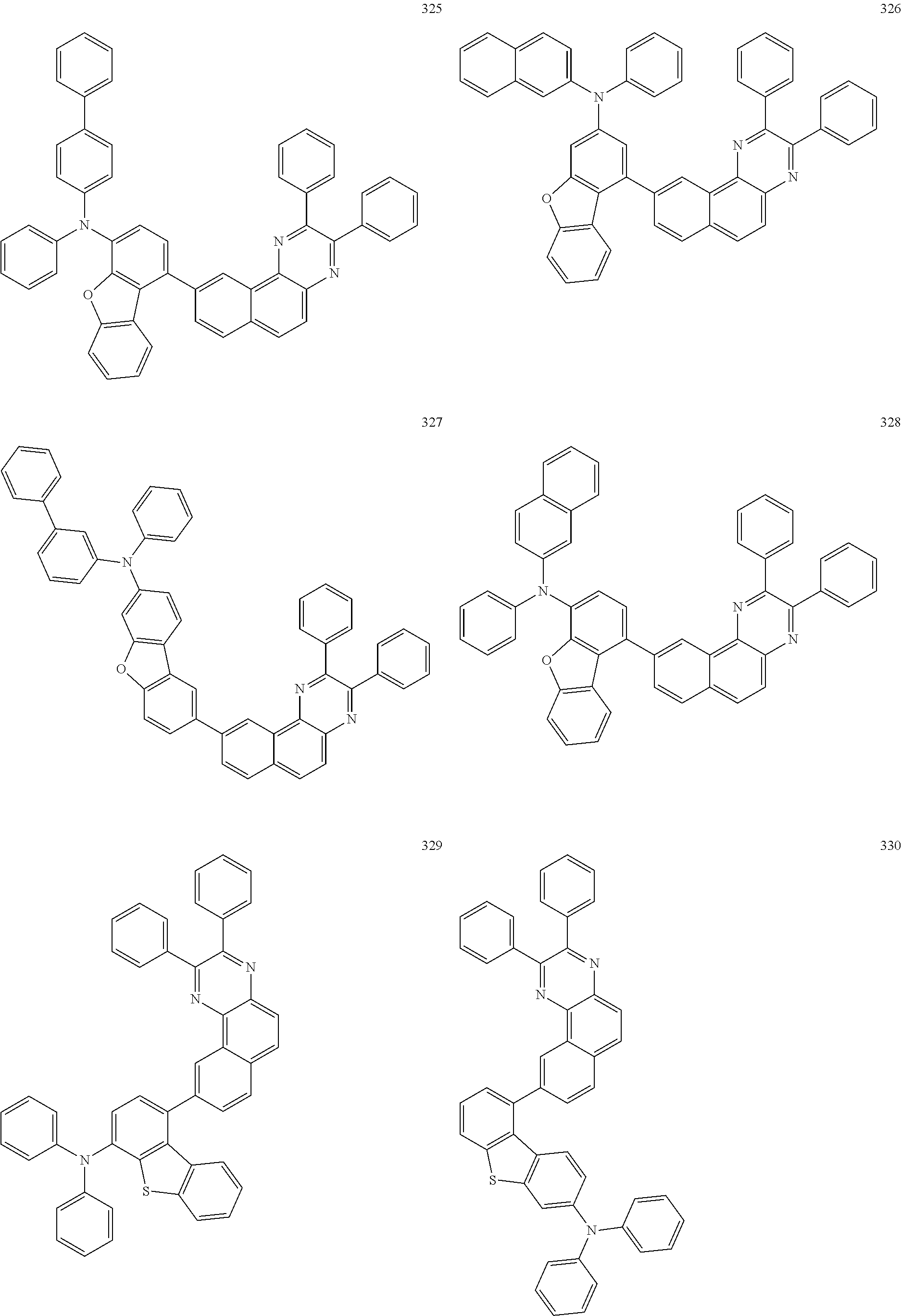

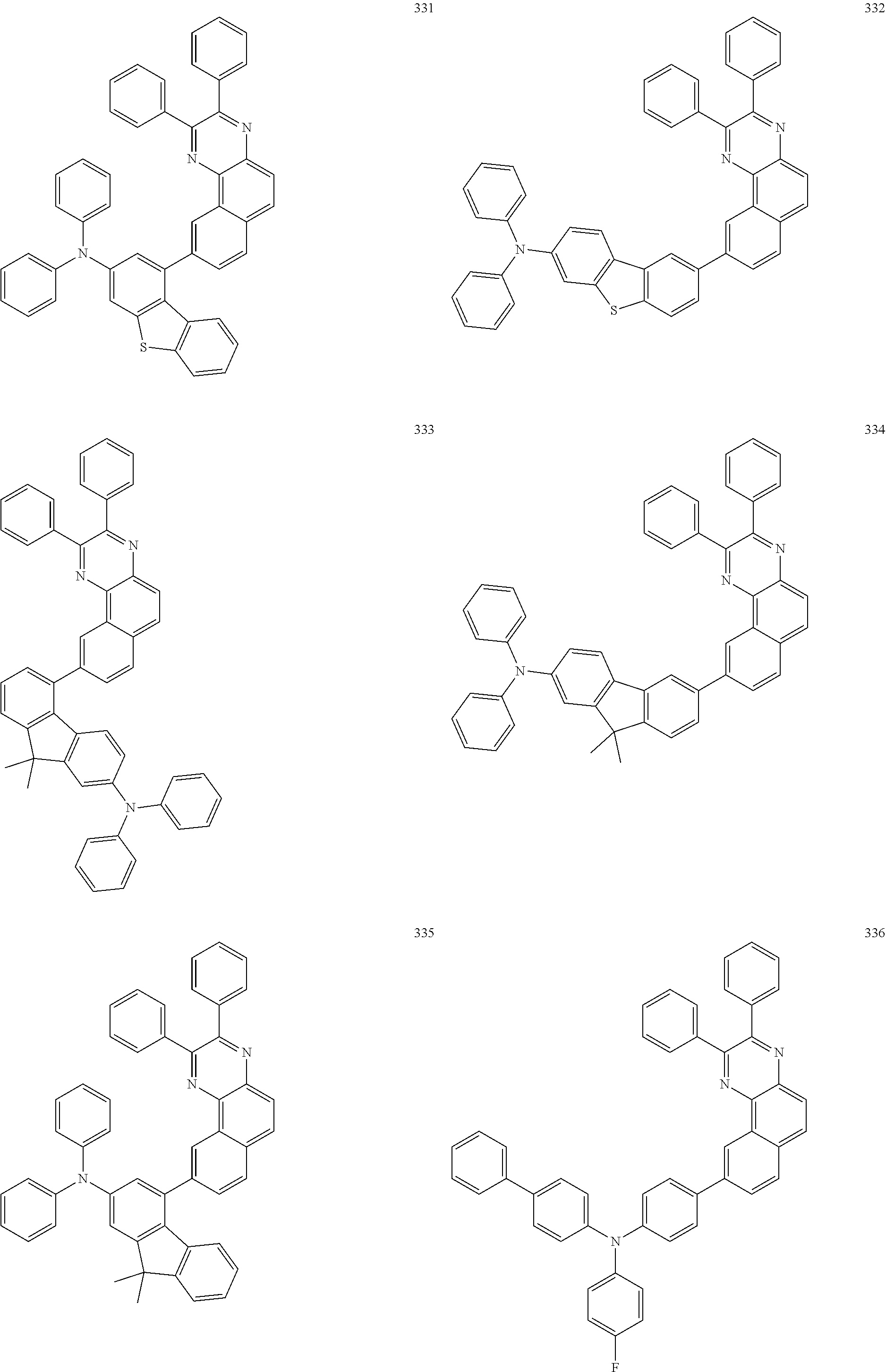

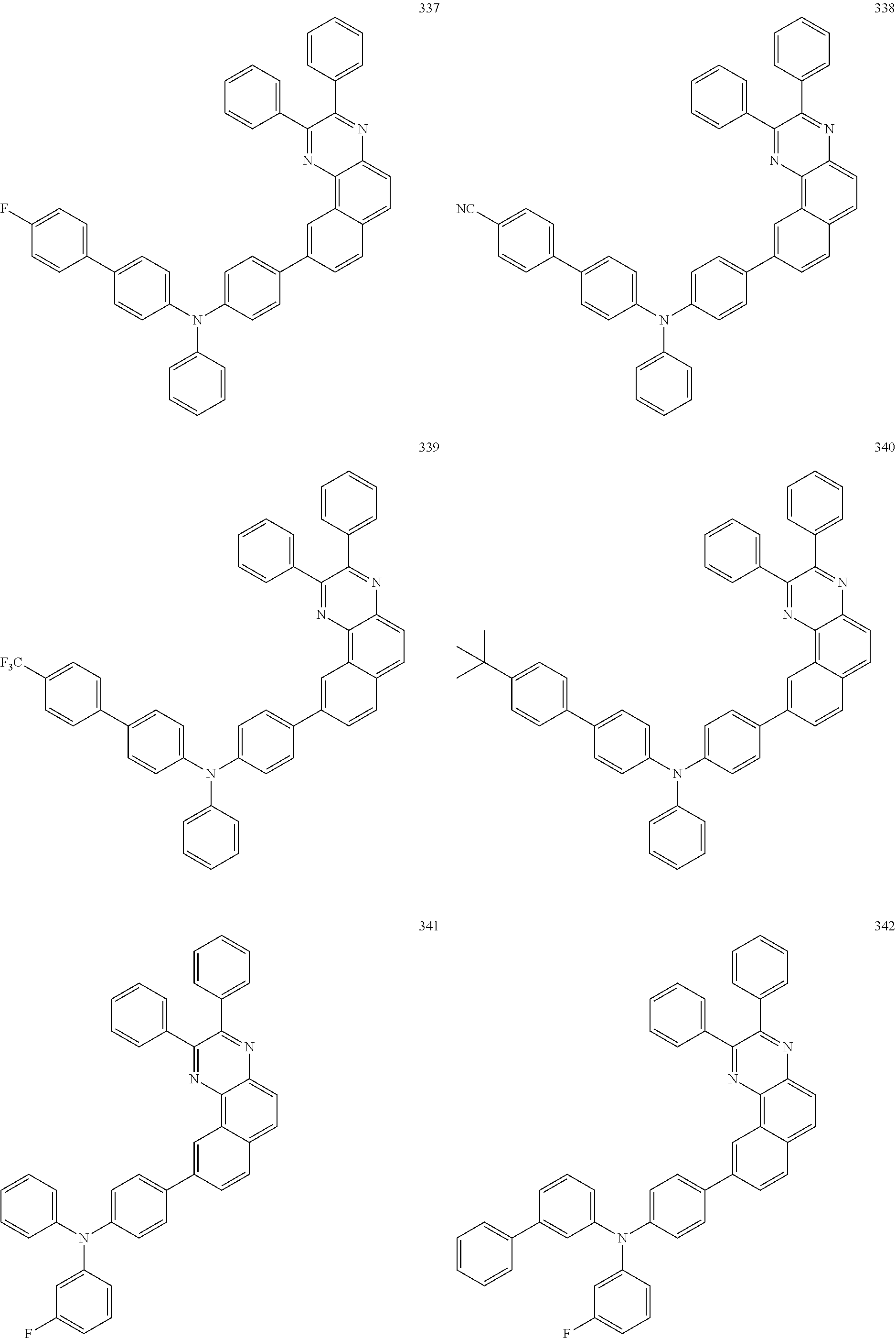

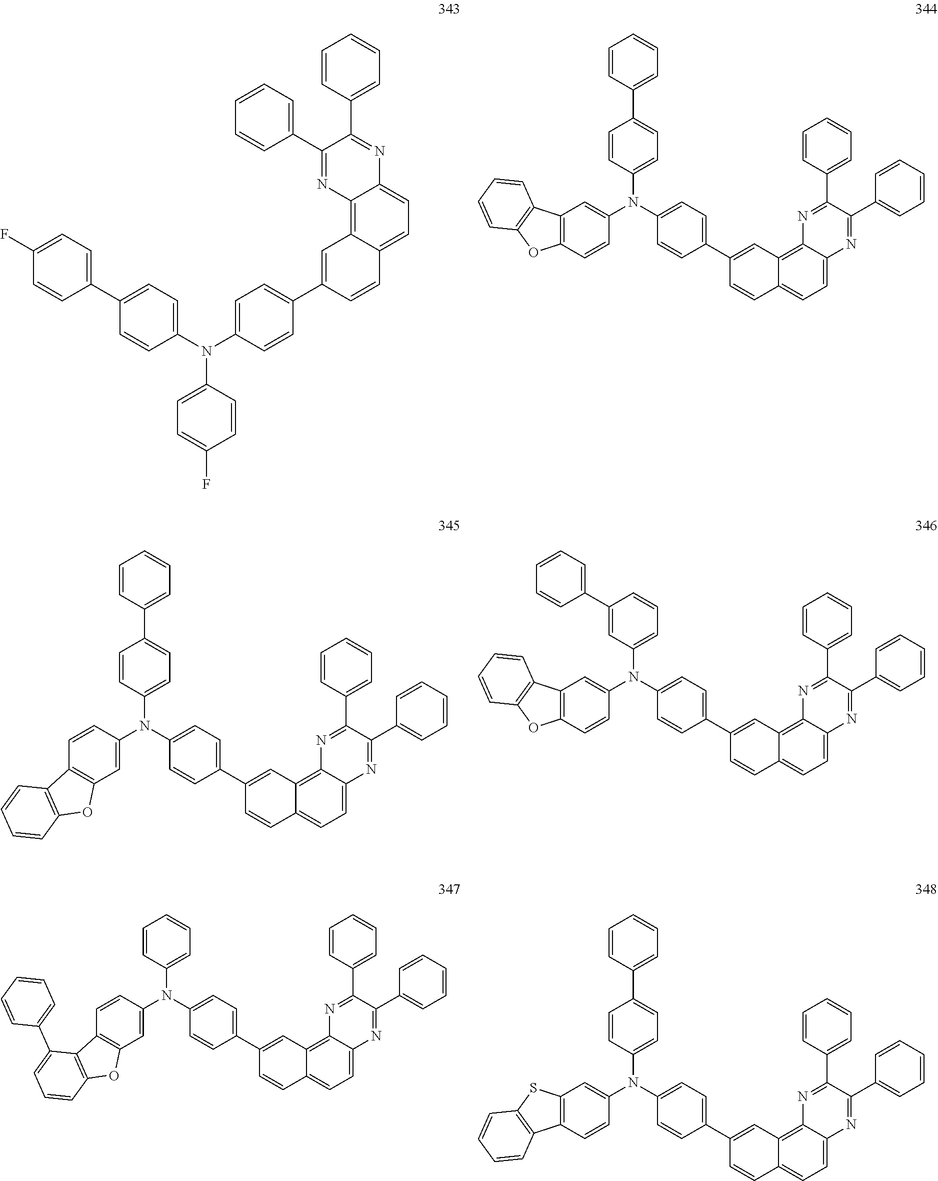

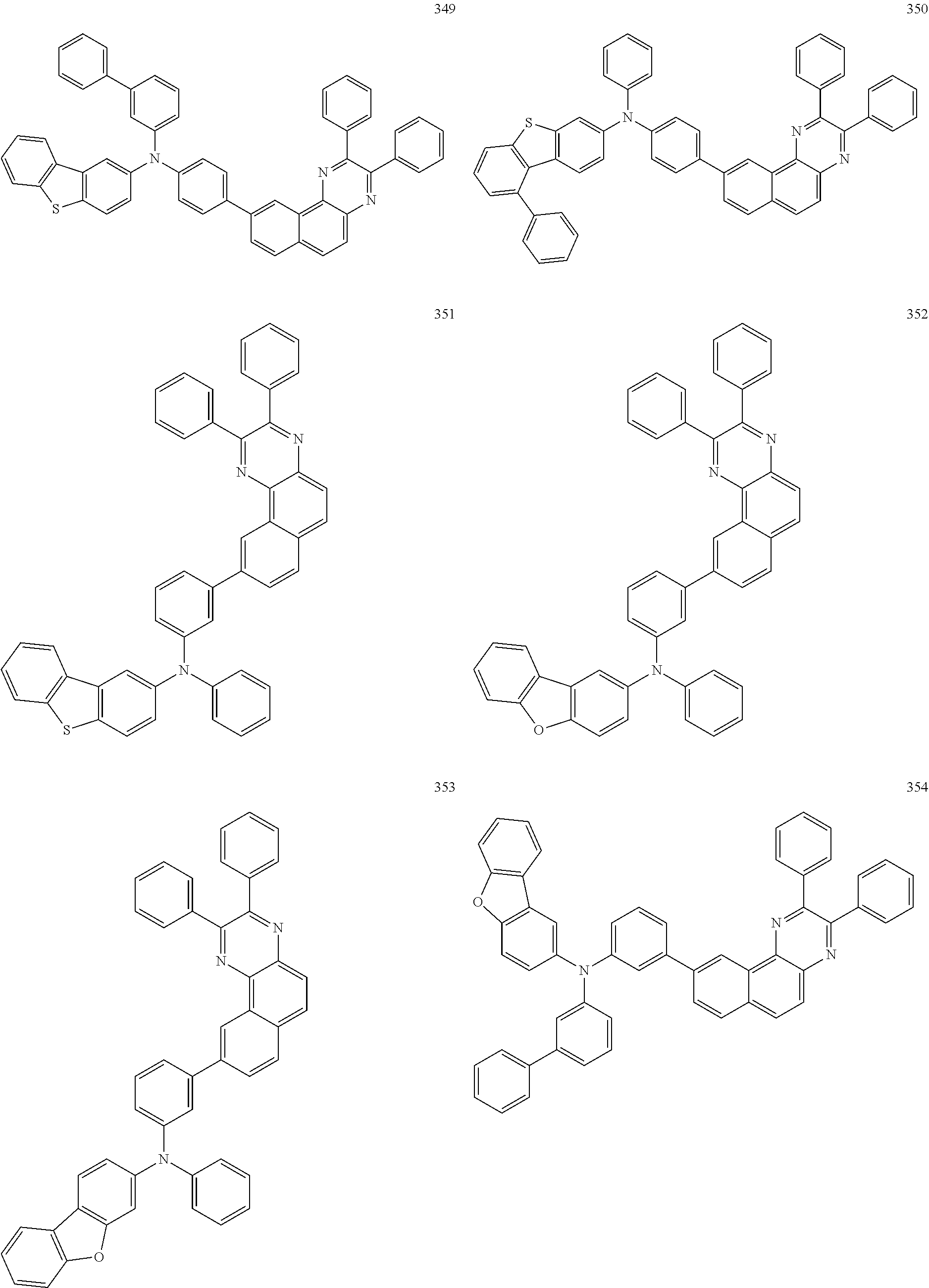

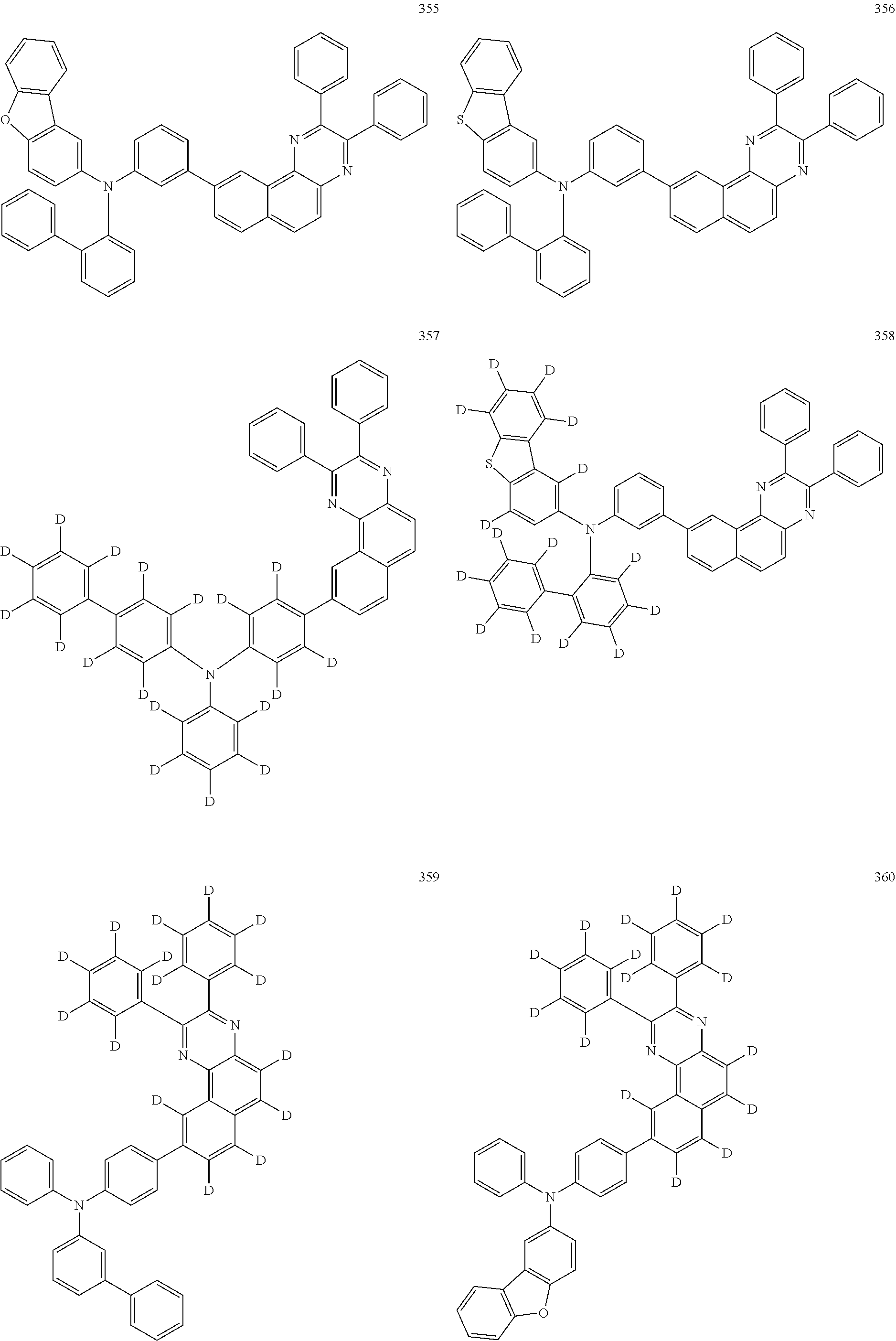

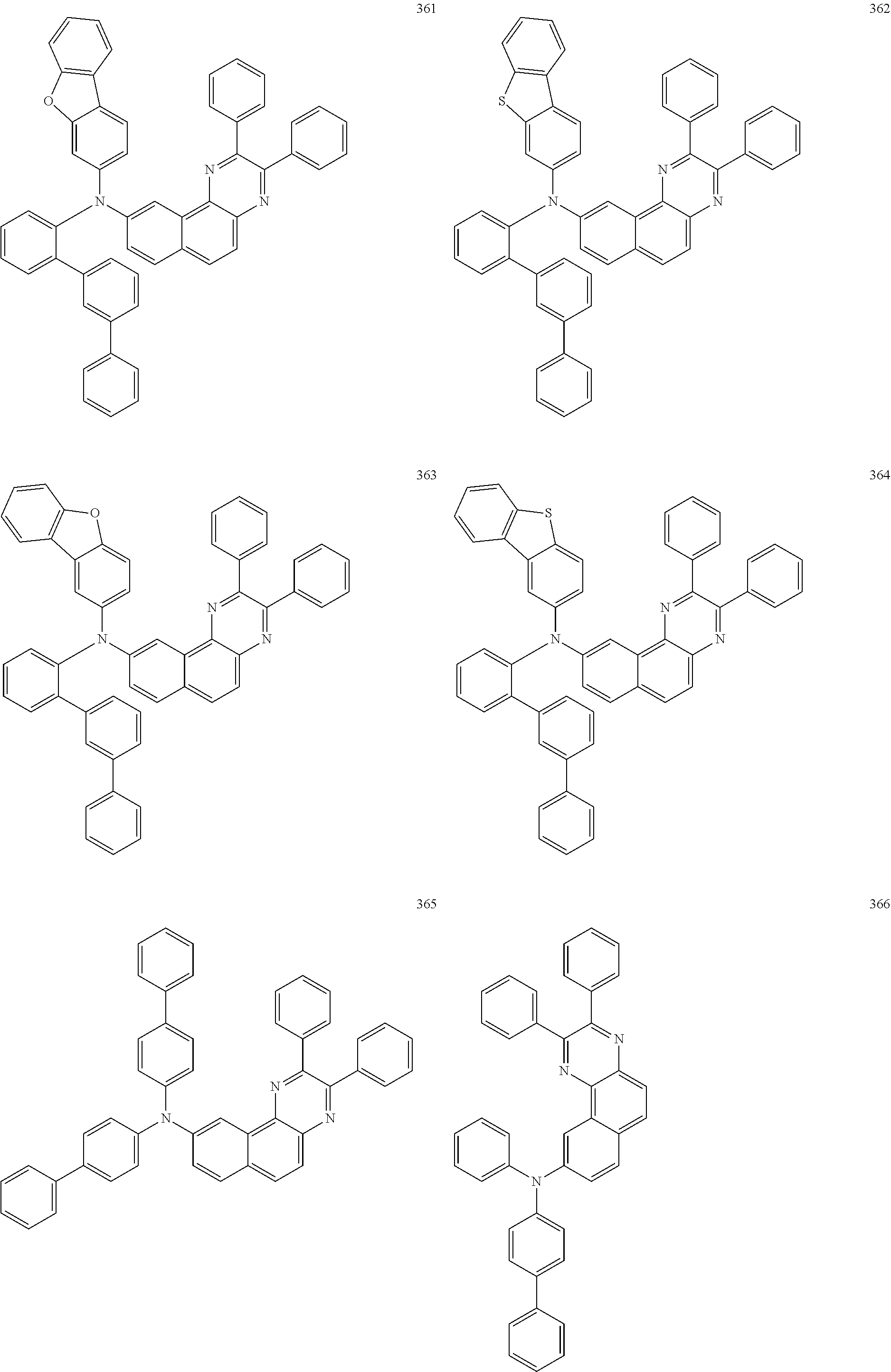

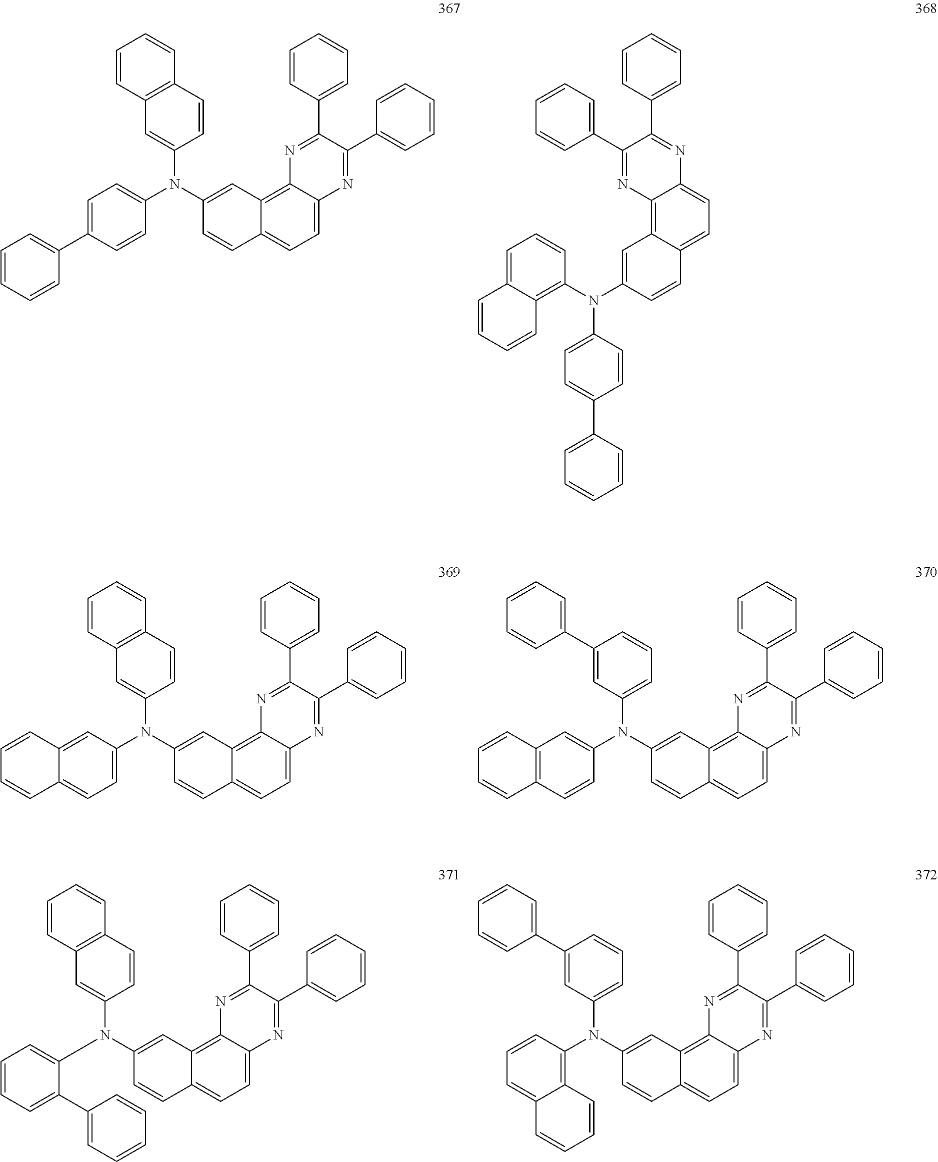 [SMILES: c1ccc(-c2ccc(N(c3ccc4ccccc4c3)c3ccc4ccc5nc(-c6ccccc6)c(-c6ccccc6)nc5c4c3)cc2)cc1 ; c1ccc(-c2ccc(N(c3ccc4ccc5nc(-c6ccccc6)c(-c6ccccc6)nc5c4c3)c3cccc4ccccc34)cc2)cc1 ; c1ccc(-c2nc3ccc4ccc(N(c5ccc6ccccc6c5)c5ccc6ccccc6c5)cc4c3nc2-c2ccccc2)cc1 ; c1ccc(-c2cccc(N(c3ccc4ccccc4c3)c3ccc4ccc5nc(-c6ccccc6)c(-c6ccccc6)nc5c4c3)c2)cc1 ; c1ccc(-c2ccccc2N(c2ccc3ccccc3c2)c2ccc3ccc4nc(-c5ccccc5)c(-c5ccccc5)nc4c3c2)cc1 ; c1ccc(-c2cccc(N(c3ccc4ccc5nc(-c6ccccc6)c(-c6ccccc6)nc5c4c3)c3cccc4ccccc34)c2)cc1]

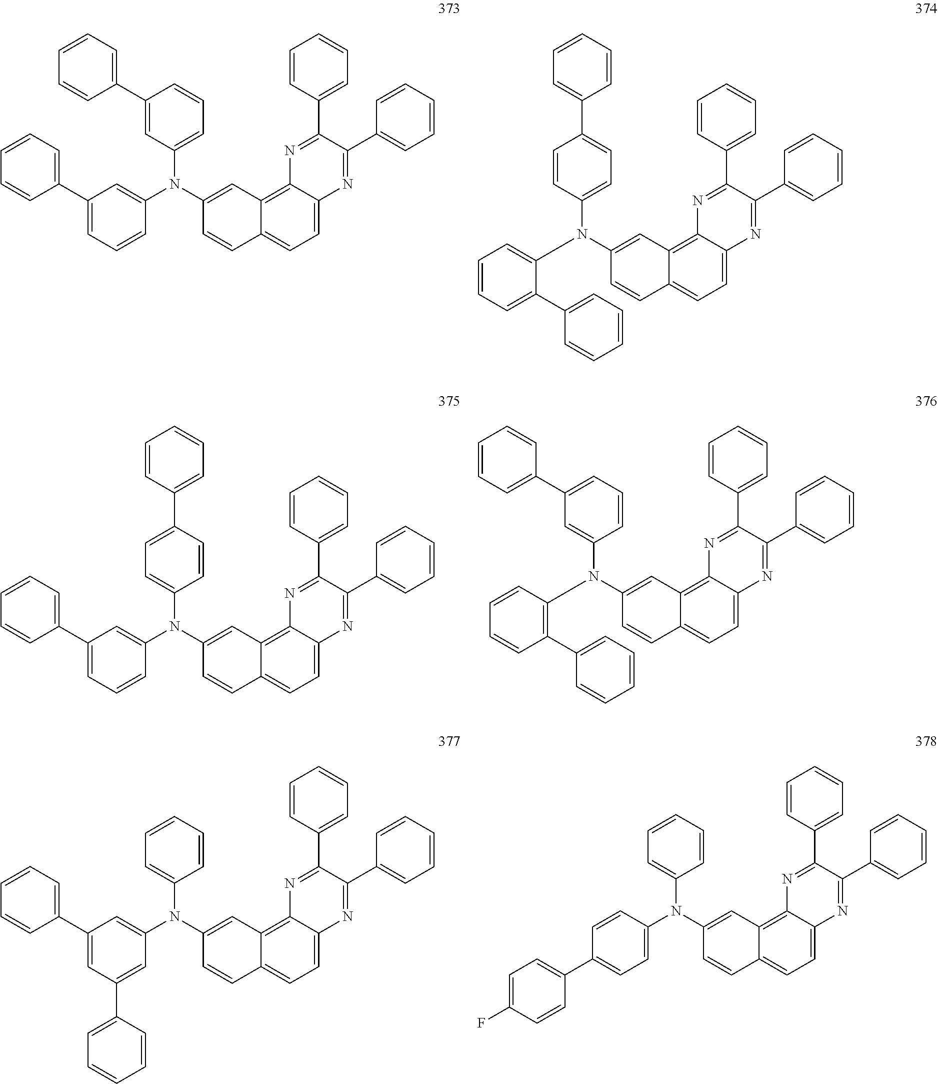

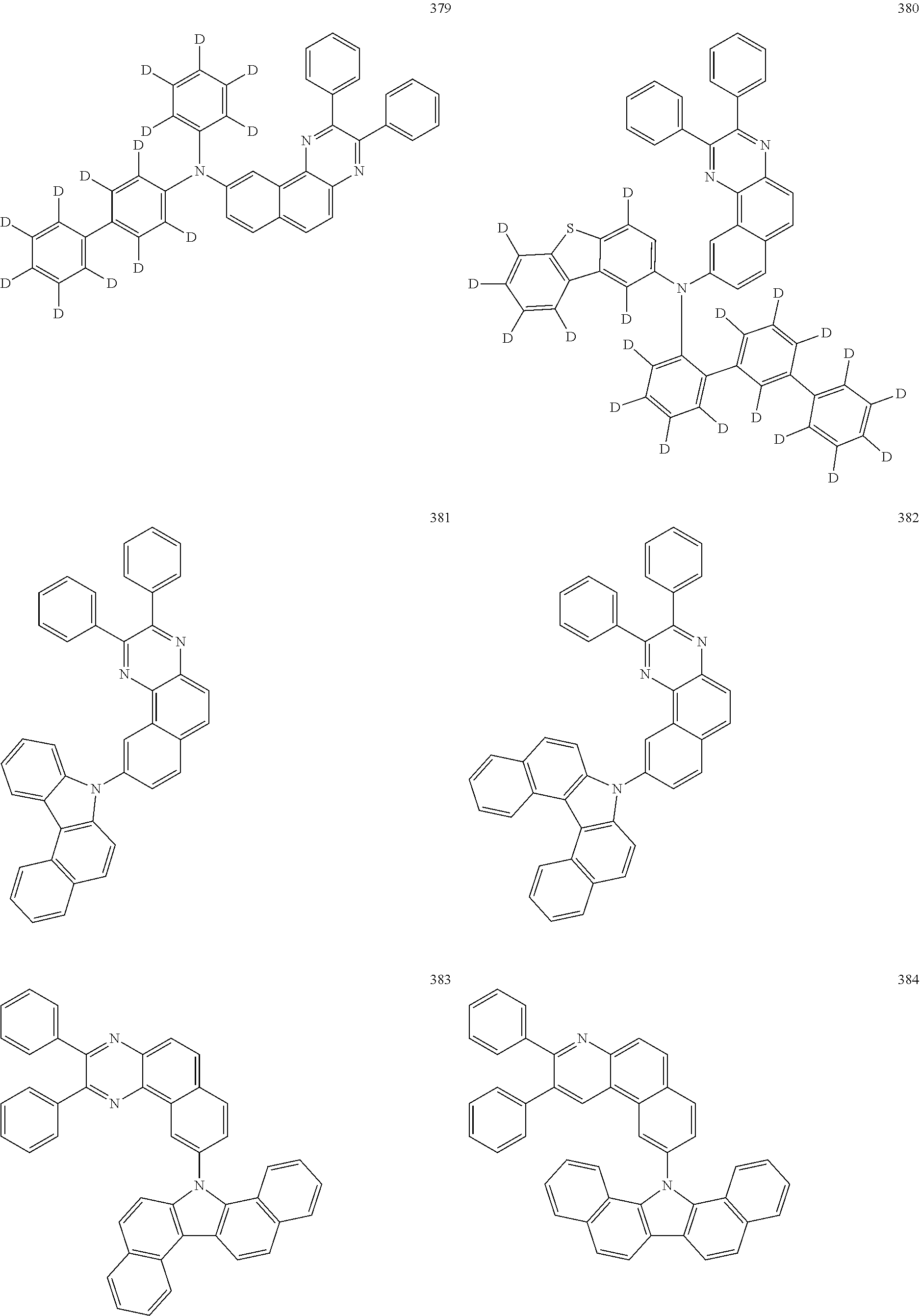

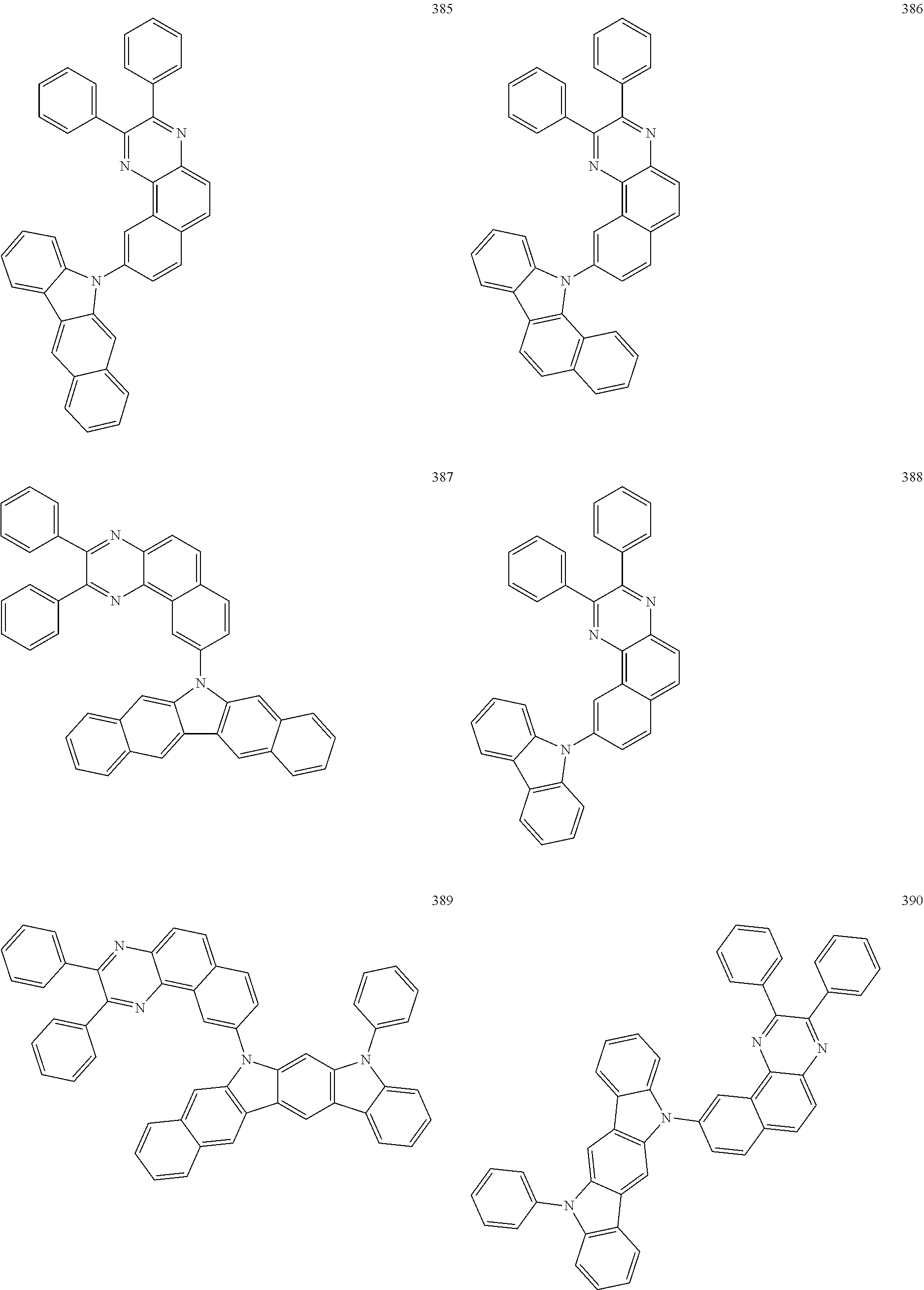 [SMILES: c1ccc(-c2nc3ccc4ccc(-n5c6ccccc6c6cc7ccccc7cc65)cc4c3nc2-c2ccccc2)cc1 ; c1ccc(-c2nc3ccc4ccc(-n5c6ccccc6c6ccc7ccccc7c65)cc4c3nc2-c2ccccc2)cc1 ; c1ccc(-c2nc3ccc4ccc(-n5c6cc7ccccc7cc6c6cc7ccccc7cc65)cc4c3nc2-c2ccccc2)cc1 ; c1ccc(-c2nc3ccc4ccc(-n5c6ccccc6c6ccccc65)cc4c3nc2-c2ccccc2)cc1 ; c1ccc(-c2nc3ccc4ccc(-n5c6cc7ccccc7cc6c6cc7c8ccccc8n(-c8ccccc8)c7cc65)cc4c3nc2-c2ccccc2)cc1 ; c1ccc(-c2nc3ccc4ccc(-n5c6ccccc6c6cc7c(cc65)c5ccccc5n7-c5ccccc5)cc4c3nc2-c2ccccc2)cc1]

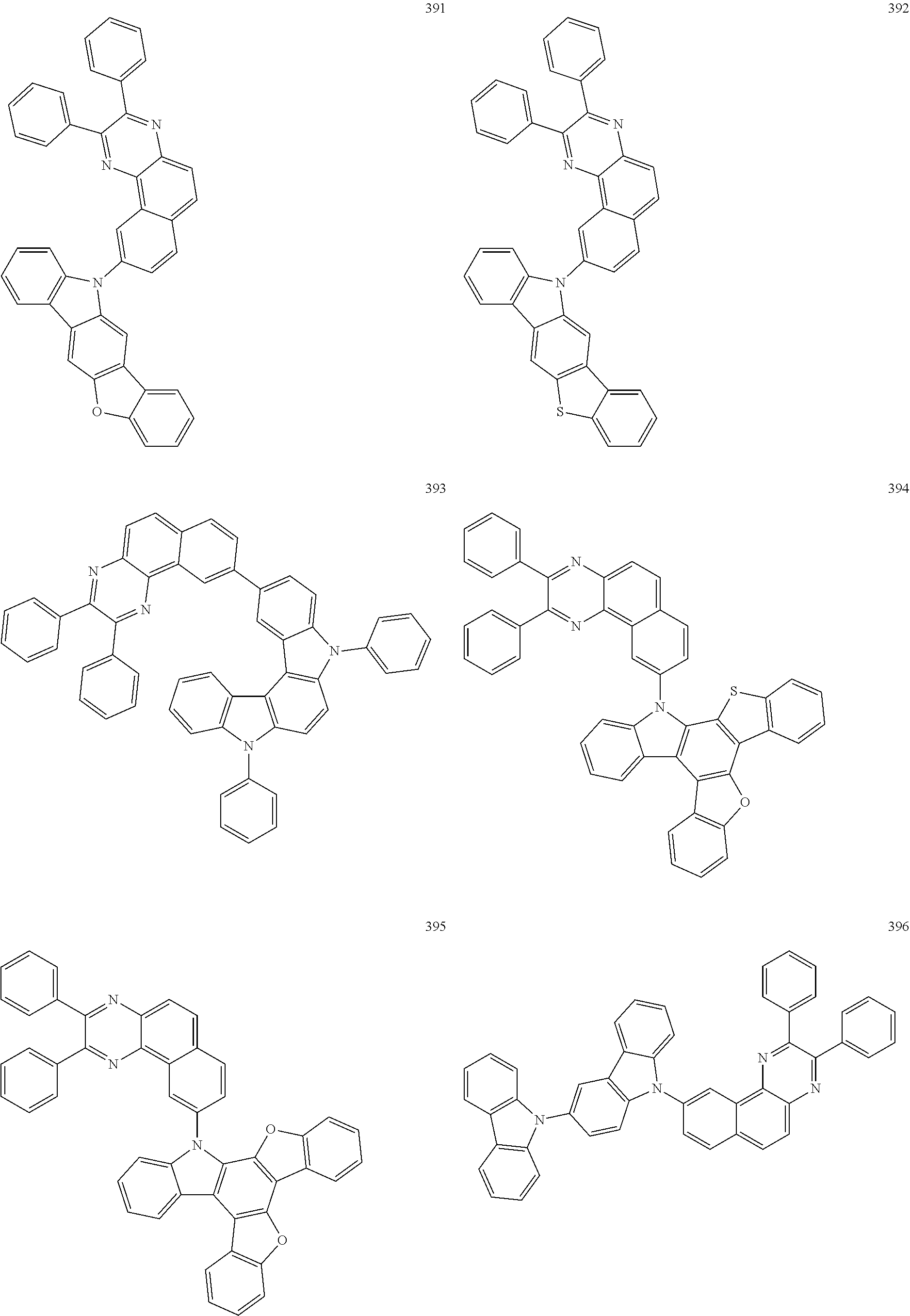

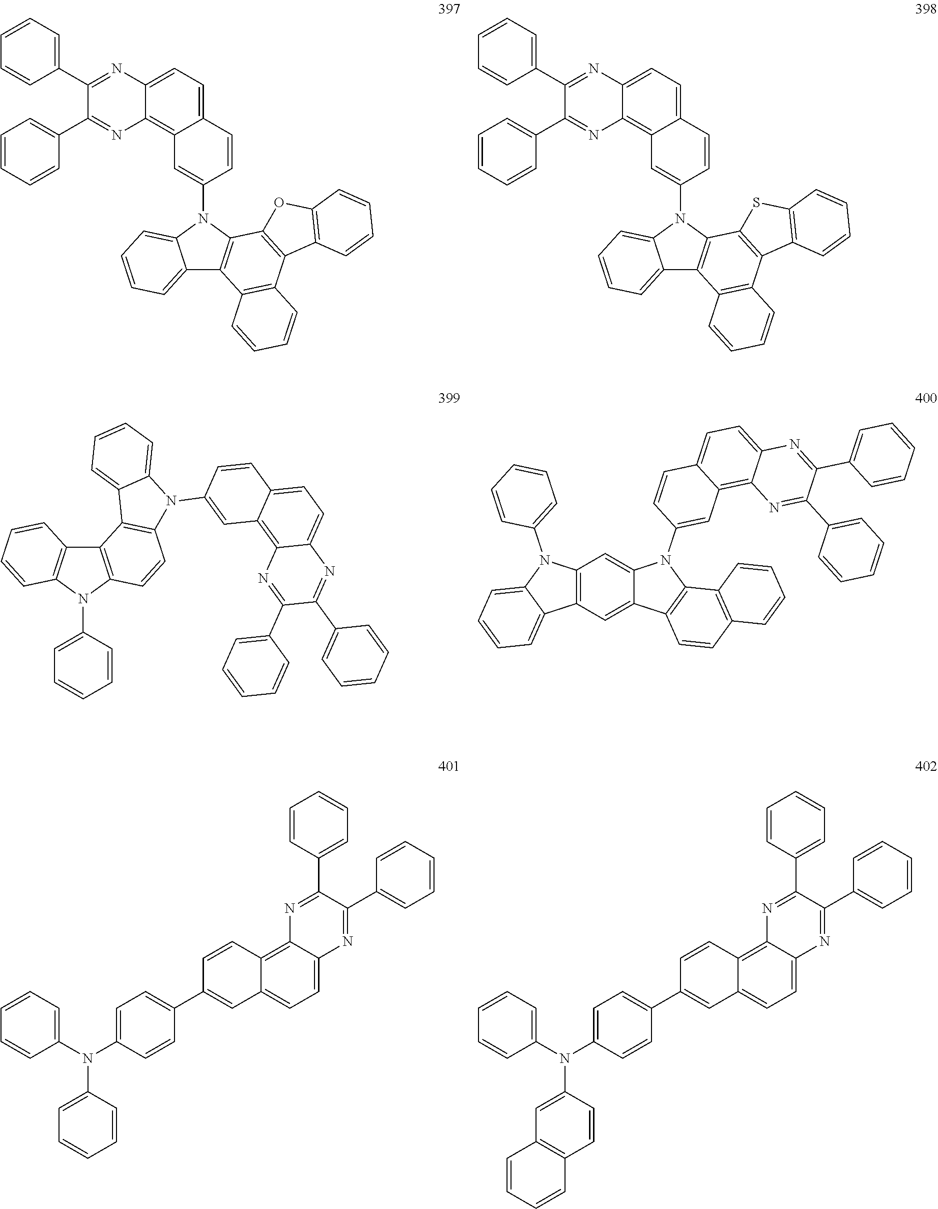 [SMILES: c1ccc(-c2nc3ccc4ccc(-n5c6ccccc6c6c7ccccc7c7c8ccccc8oc7c65)cc4c3nc2-c2ccccc2)cc1 ; c1ccc(-c2nc3ccc4ccc(-n5c6ccccc6c6c7ccccc7c7c8ccccc8sc7c65)cc4c3nc2-c2ccccc2)cc1 ; c1ccc(-c2nc3ccc4ccc(-n5c6ccccc6c6c7c8ccccc8n(-c8ccccc8)c7ccc65)cc4c3nc2-c2ccccc2)cc1 ; c1ccc(-c2nc3ccc4ccc(-n5c6cc7c(cc6c6ccc8ccccc8c65)c5ccccc5n7-c5ccccc5)cc4c3nc2-c2ccccc2)cc1 ; c1ccc(-c2nc3ccc4cc(-c5ccc(N(c6ccccc6)c6ccccc6)cc5)ccc4c3nc2-c2ccccc2)cc1 ; c1ccc(-c2nc3ccc4cc(-c5ccc(N(c6ccccc6)c6ccc7ccccc7c6)cc5)ccc4c3nc2-c2ccccc2)cc1]

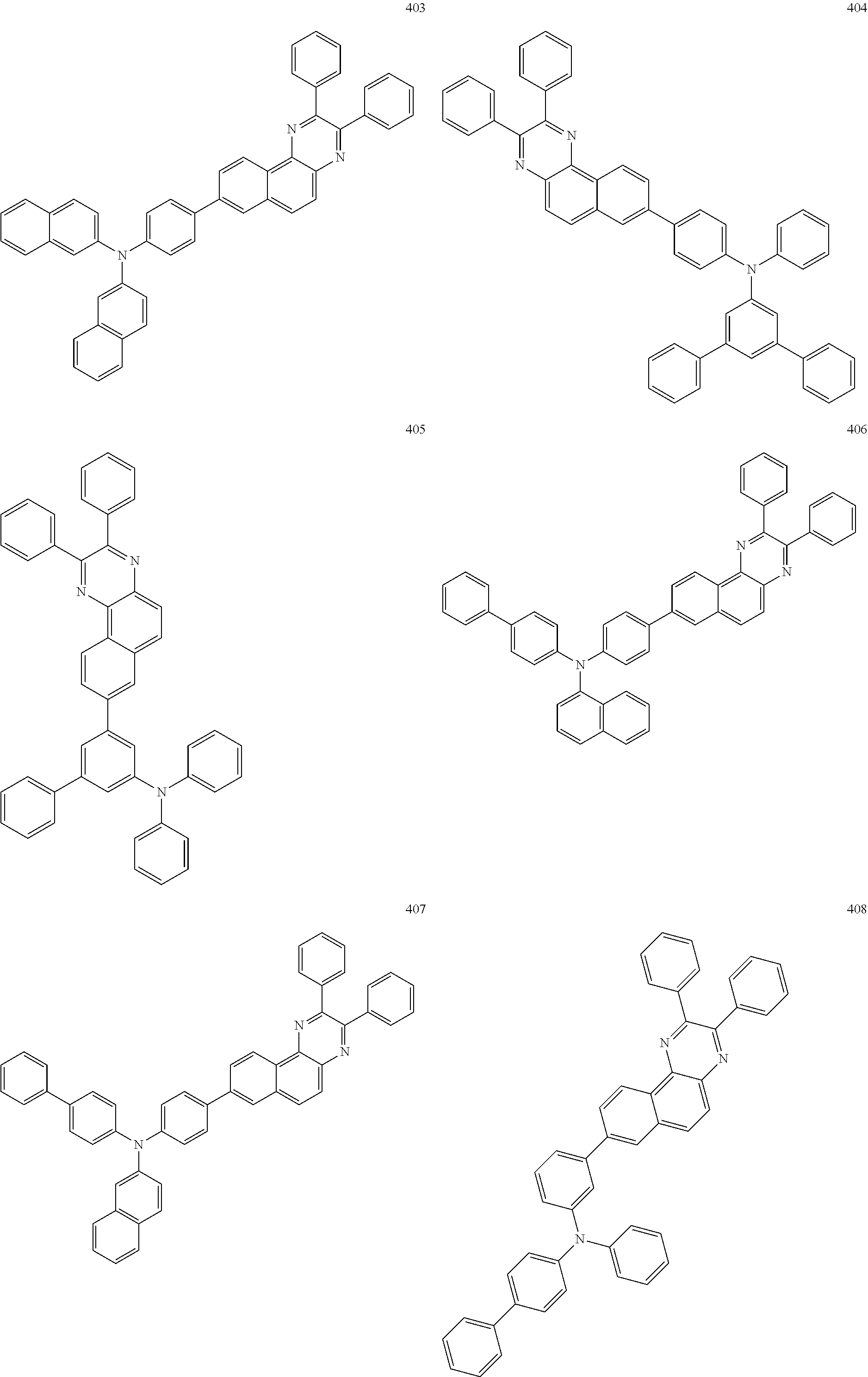

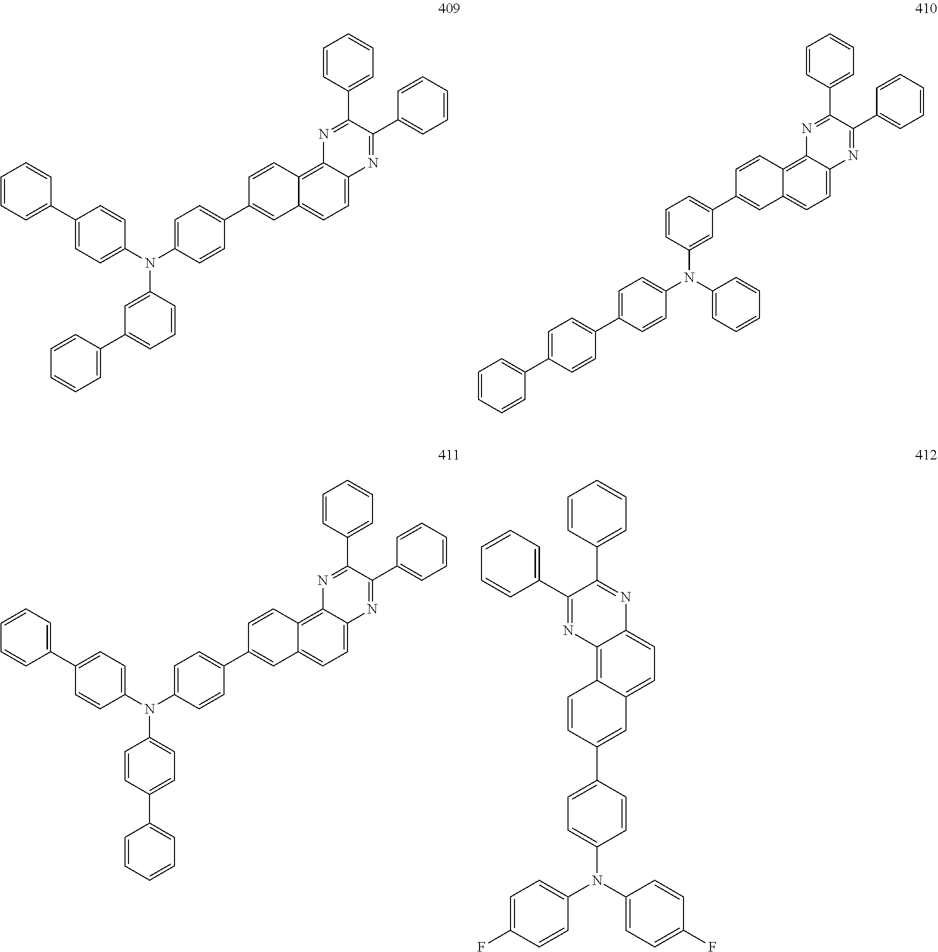

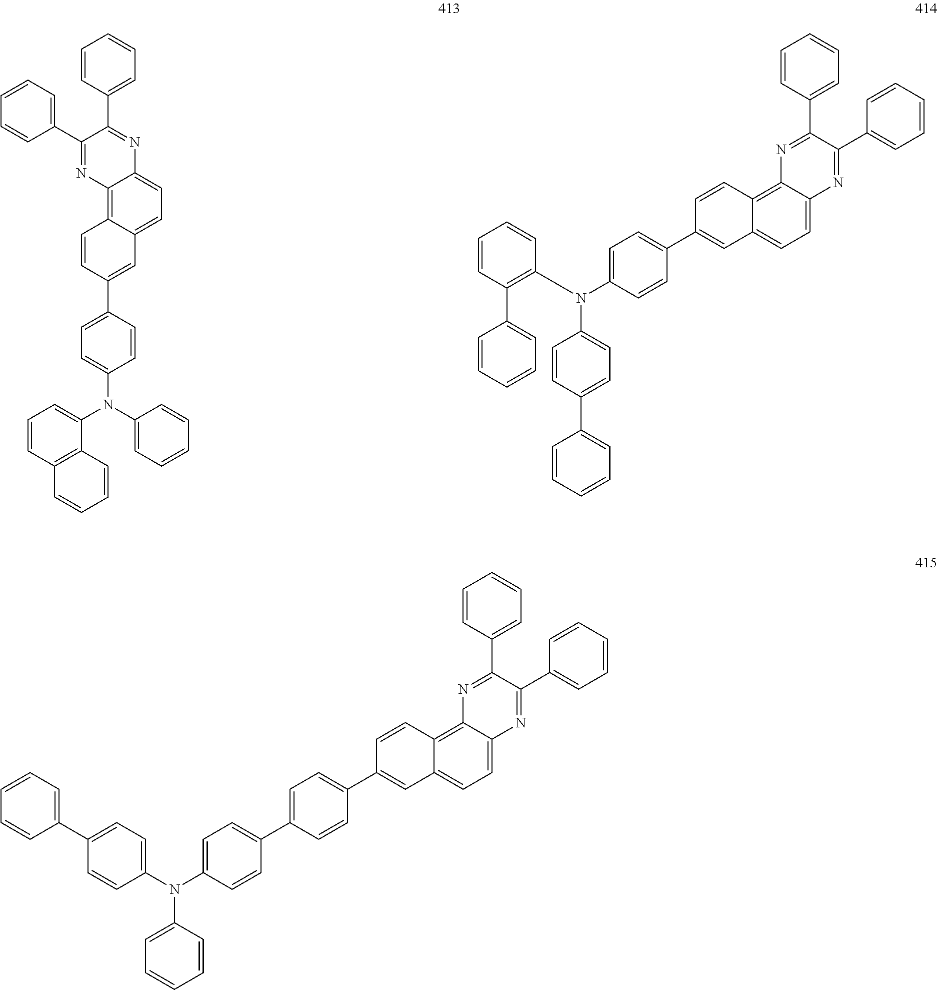 [SMILES: c1ccc(-c2nc3ccc4cc(-c5ccc(N(c6ccccc6)c6cccc7ccccc67)cc5)ccc4c3nc2-c2ccccc2)cc1 ; c1ccc(-c2ccc(N(c3ccc(-c4ccc5c(ccc6nc(-c7ccccc7)c(-c7ccccc7)nc65)c4)cc3)c3ccccc3-c3ccccc3)cc2)cc1 ; c1ccc(-c2ccc(N(c3ccccc3)c3ccc(-c4ccc(-c5ccc6c(ccc7nc(-c8ccccc8)c(-c8ccccc8)nc76)c5)cc4)cc3)cc2)cc1]

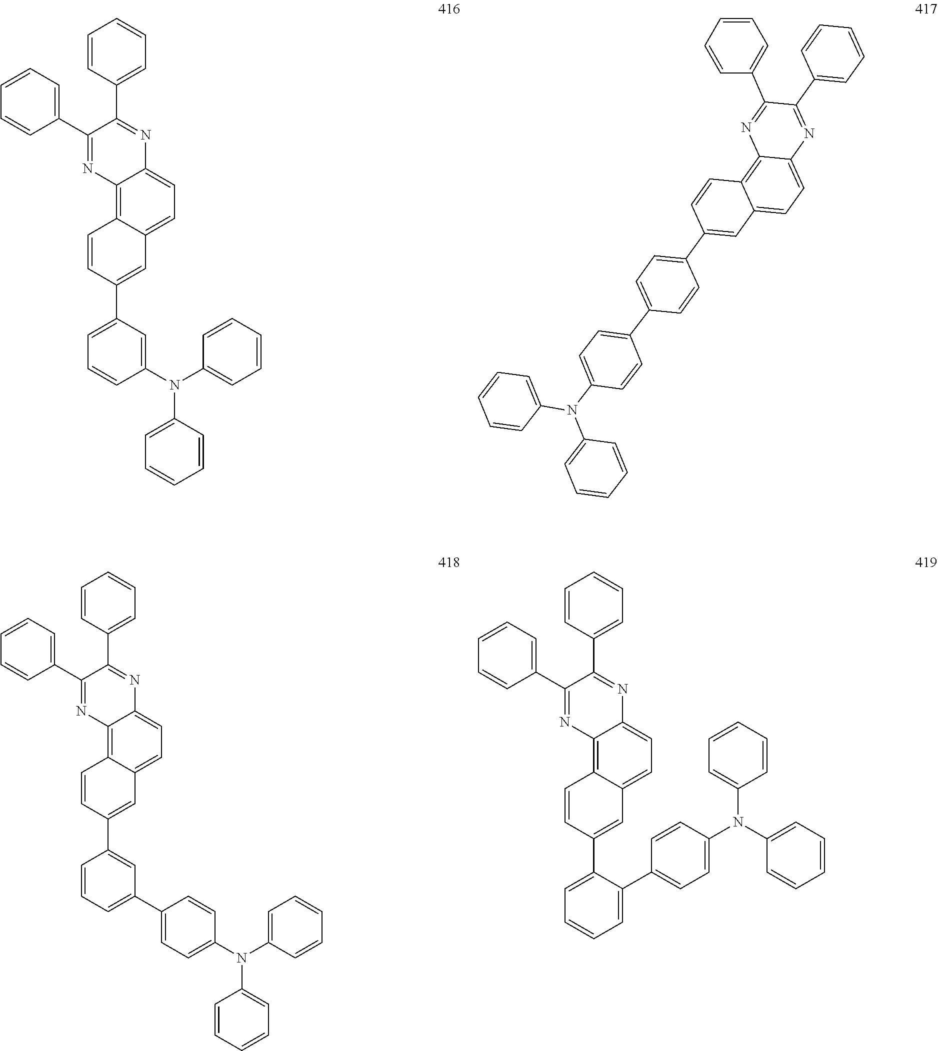 [SMILES: c1ccc(-c2nc3ccc4cc(-c5cccc(N(c6ccccc6)c6ccccc6)c5)ccc4c3nc2-c2ccccc2)cc1 ; c1ccc(-c2nc3ccc4cc(-c5ccc(-c6ccc(N(c7ccccc7)c7ccccc7)cc6)cc5)ccc4c3nc2-c2ccccc2)cc1 ; c1ccc(-c2nc3ccc4cc(-c5cccc(-c6ccc(N(c7ccccc7)c7ccccc7)cc6)c5)ccc4c3nc2-c2ccccc2)cc1 ; c1ccc(-c2nc3ccc4cc(-c5ccccc5-c5ccc(N(c6ccccc6)c6ccccc6)cc5)ccc4c3nc2-c2ccccc2)cc1]

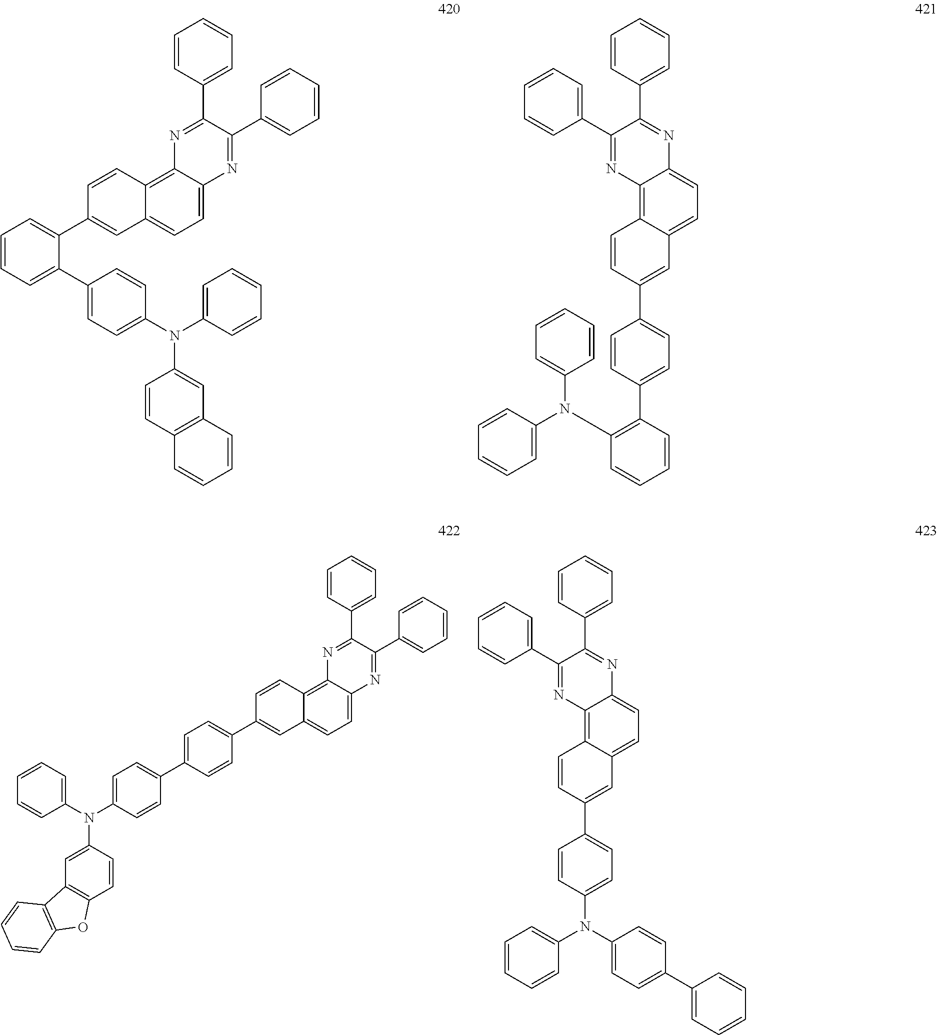

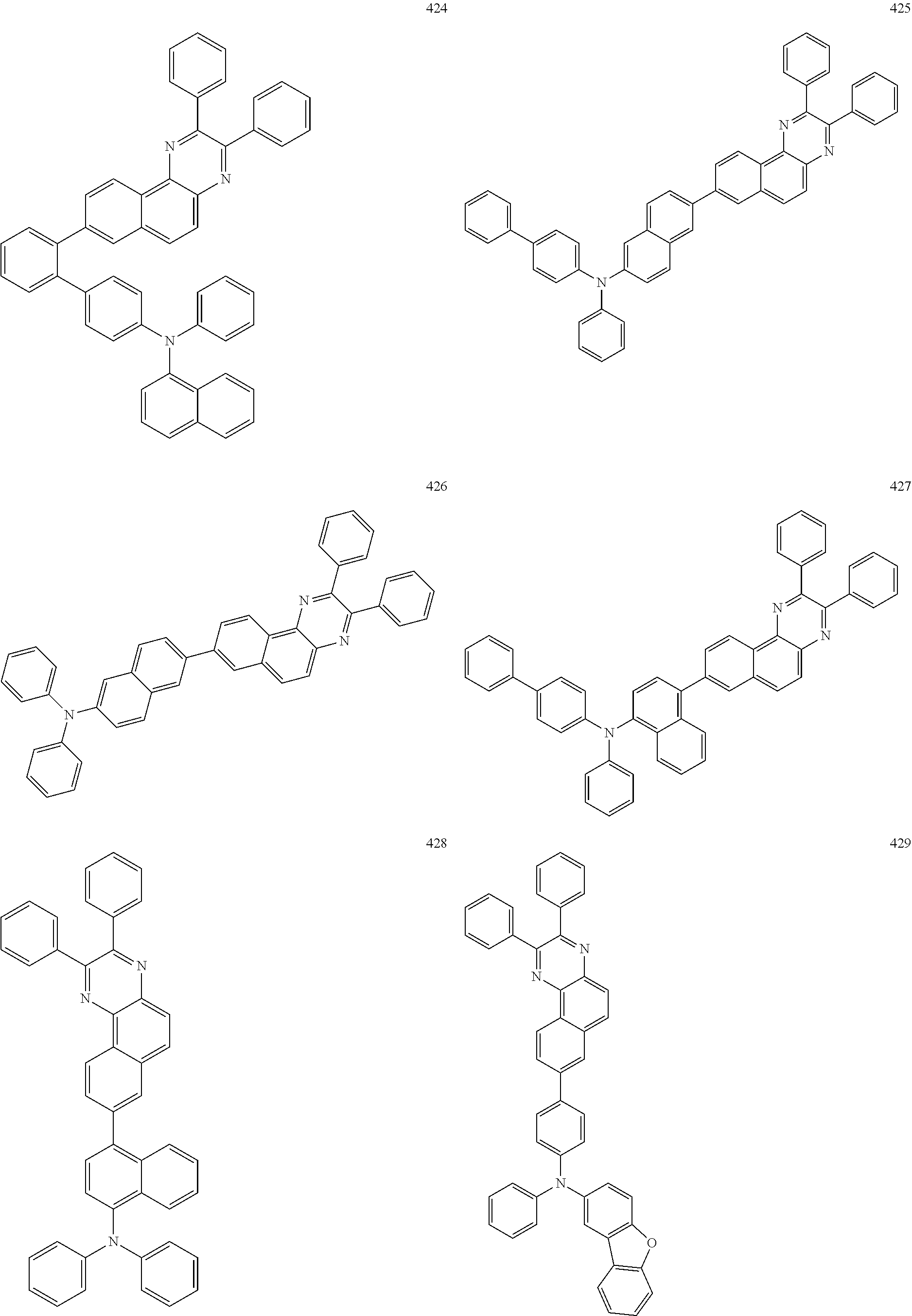

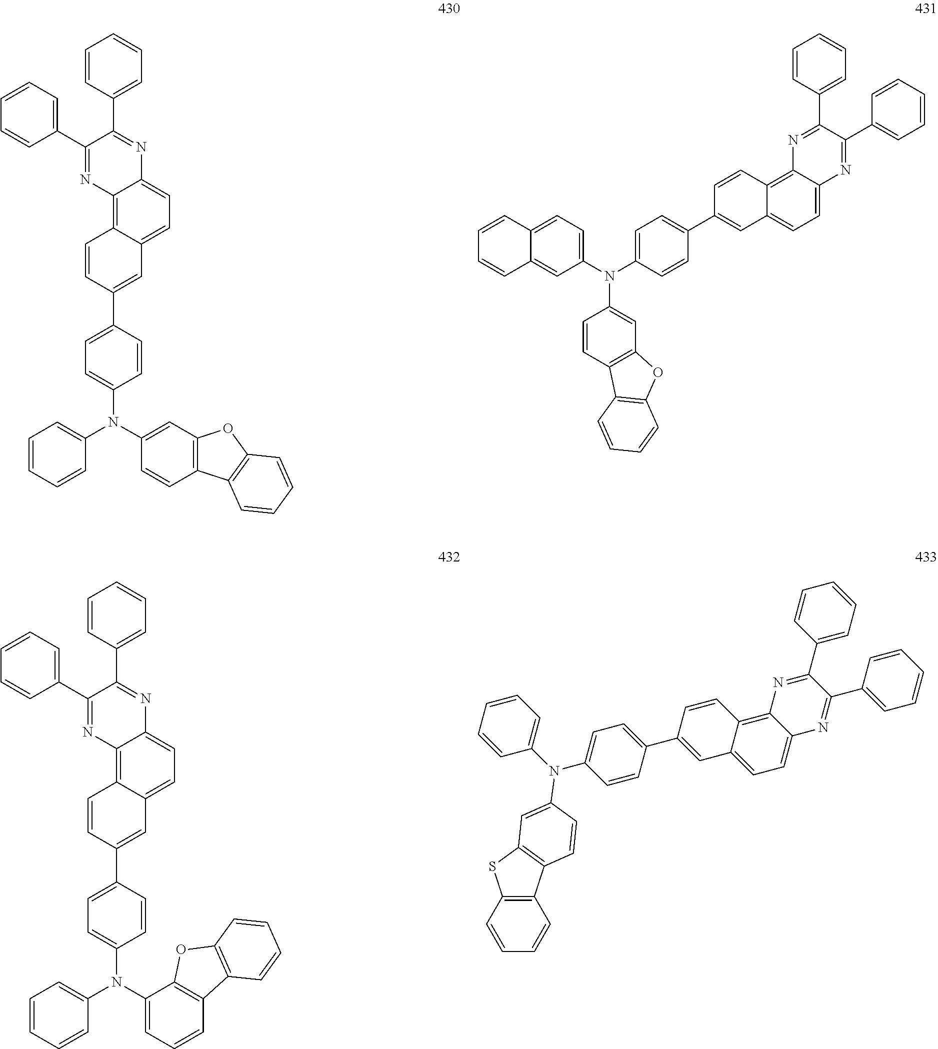 [SMILES: c1ccc(-c2nc3ccc4cc(-c5ccc(N(c6ccccc6)c6ccc7c(c6)oc6ccccc67)cc5)ccc4c3nc2-c2ccccc2)cc1 ; c1ccc(-c2nc3ccc4cc(-c5ccc(N(c6ccc7ccccc7c6)c6ccc7c(c6)oc6ccccc67)cc5)ccc4c3nc2-c2ccccc2)cc1 ; c1ccc(-c2nc3ccc4cc(-c5ccc(N(c6ccccc6)c6cccc7c6oc6ccccc67)cc5)ccc4c3nc2-c2ccccc2)cc1 ; c1ccc(-c2nc3ccc4cc(-c5ccc(N(c6ccccc6)c6ccc7c(c6)sc6ccccc67)cc5)ccc4c3nc2-c2ccccc2)cc1]

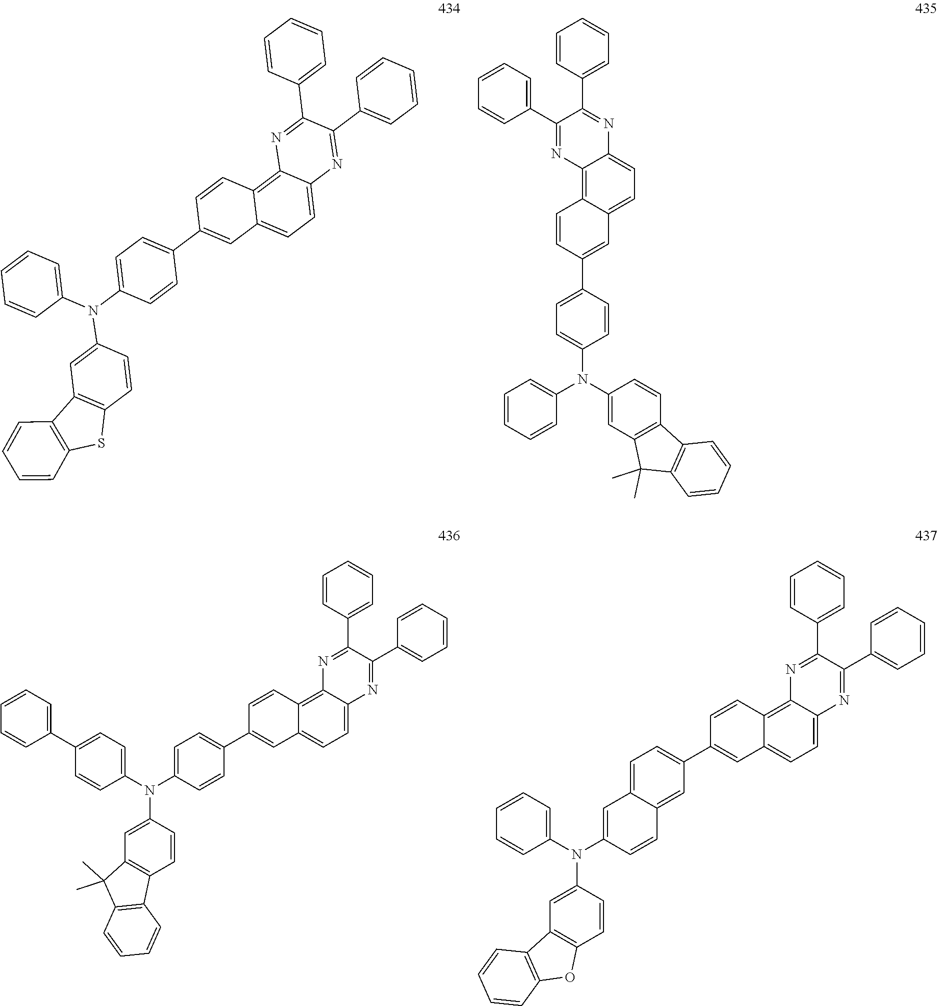 [SMILES: c1ccc(-c2nc3ccc4cc(-c5ccc(N(c6ccccc6)c6ccc7sc8ccccc8c7c6)cc5)ccc4c3nc2-c2ccccc2)cc1 ; CC1(C)c2ccccc2-c2ccc(N(c3ccccc3)c3ccc(-c4ccc5c(ccc6nc(-c7ccccc7)c(-c7ccccc7)nc65)c4)cc3)cc21 ; CC1(C)c2ccccc2-c2ccc(N(c3ccc(-c4ccccc4)cc3)c3ccc(-c4ccc5c(ccc6nc(-c7ccccc7)c(-c7ccccc7)nc65)c4)cc3)cc21 ; c1ccc(-c2nc3ccc4cc(-c5ccc6cc(N(c7ccccc7)c7ccc8oc9ccccc9c8c7)ccc6c5)ccc4c3nc2-c2ccccc2)cc1]

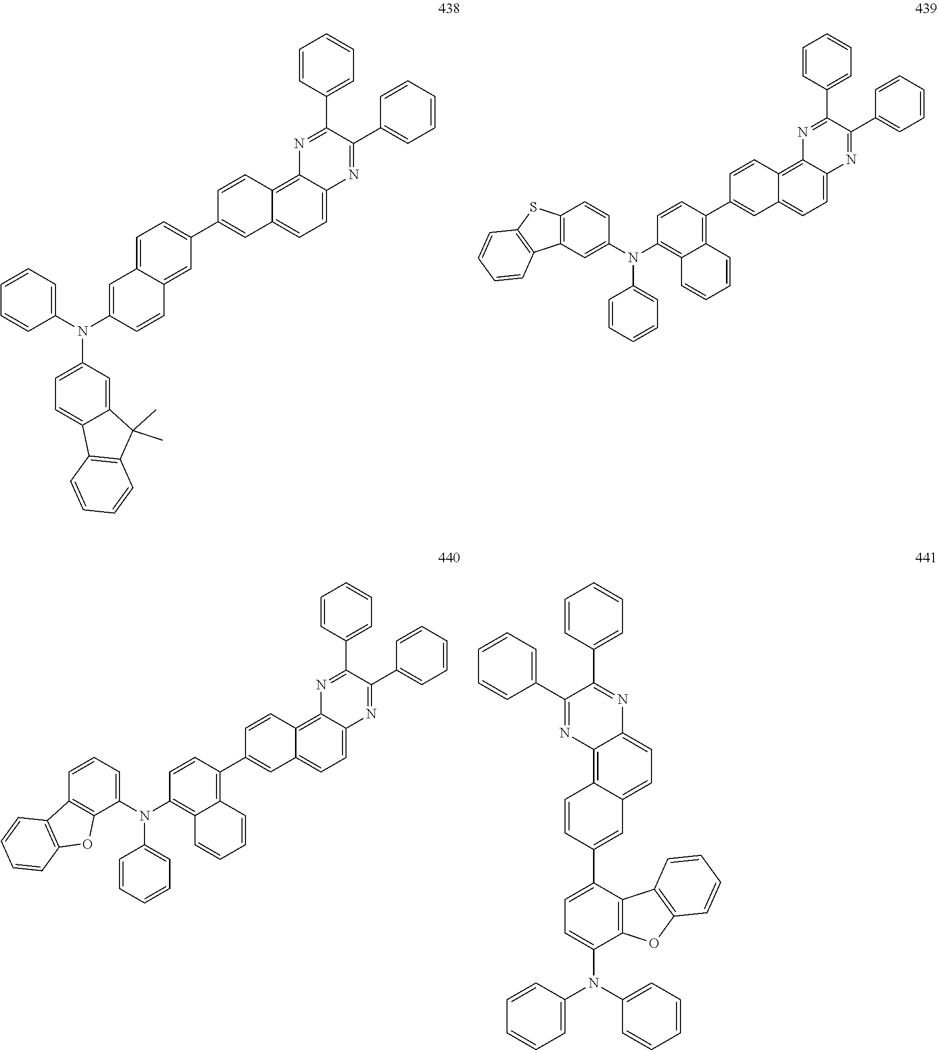

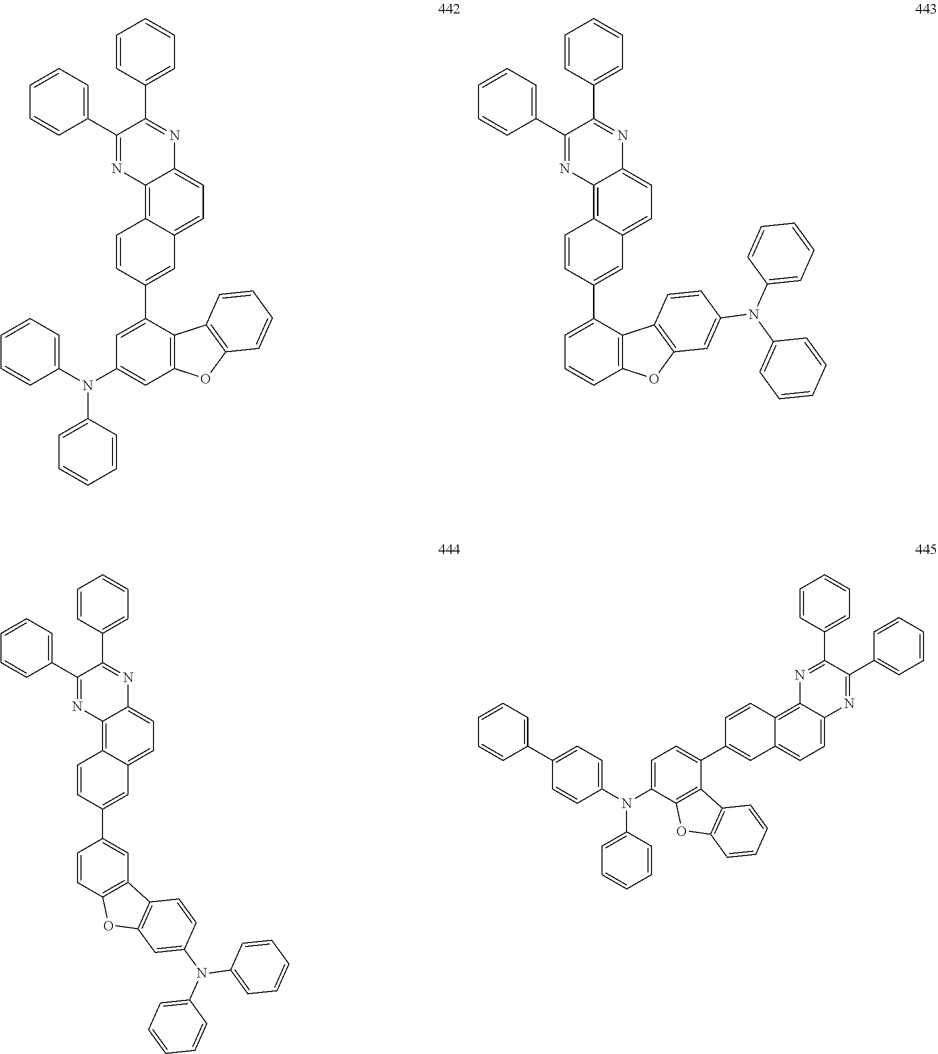 [SMILES: c1ccc(-c2nc3ccc4cc(-c5cc(N(c6ccccc6)c6ccccc6)cc6oc7ccccc7c56)ccc4c3nc2-c2ccccc2)cc1 ; c1ccc(-c2nc3ccc4cc(-c5cccc6oc7cc(N(c8ccccc8)c8ccccc8)ccc7c56)ccc4c3nc2-c2ccccc2)cc1 ; c1ccc(-c2nc3ccc4cc(-c5ccc6oc7cc(N(c8ccccc8)c8ccccc8)ccc7c6c5)ccc4c3nc2-c2ccccc2)cc1 ; c1ccc(-c2ccc(N(c3ccccc3)c3ccc(-c4ccc5c(ccc6nc(-c7ccccc7)c(-c7ccccc7)nc65)c4)c4c3oc3ccccc34)cc2)cc1]

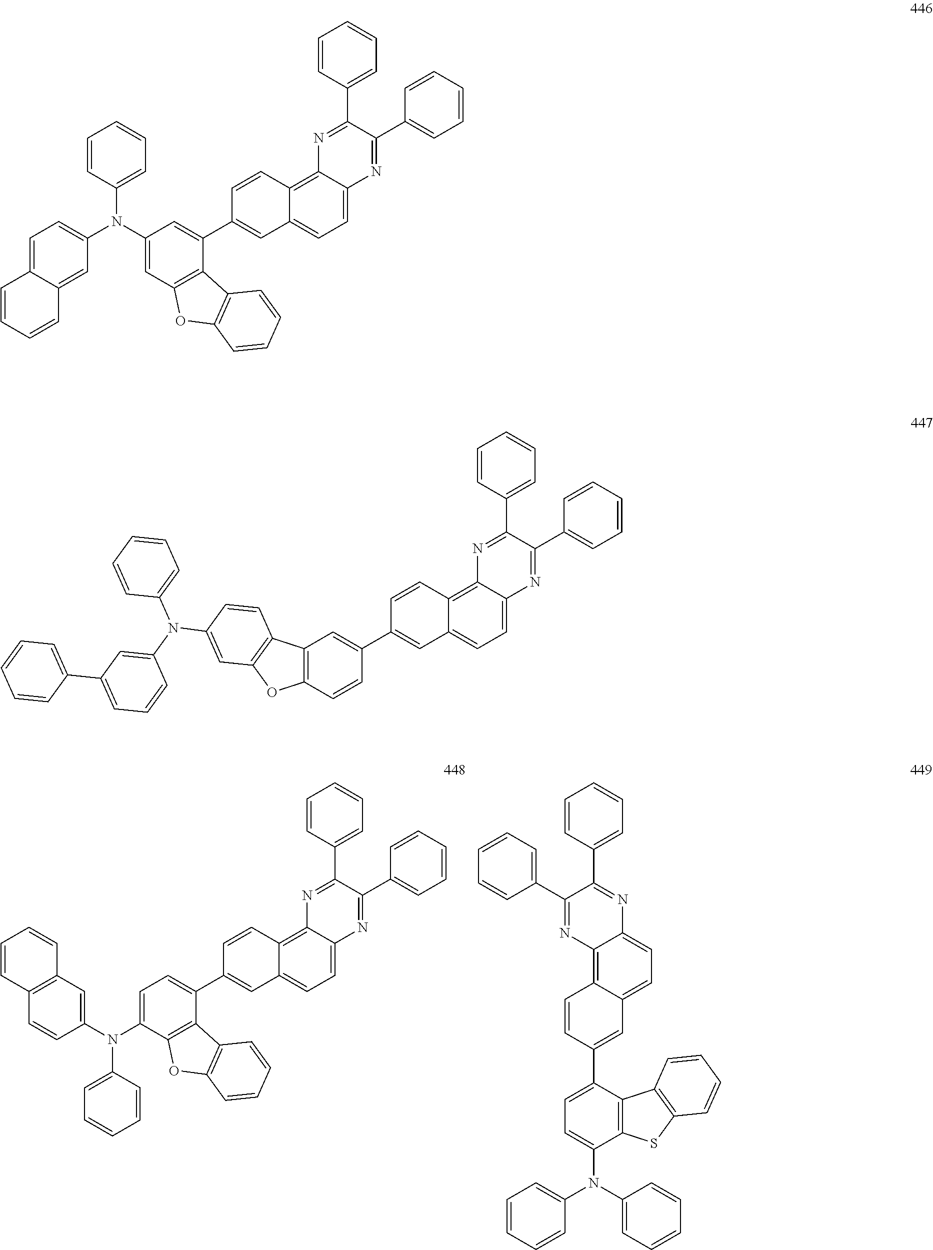

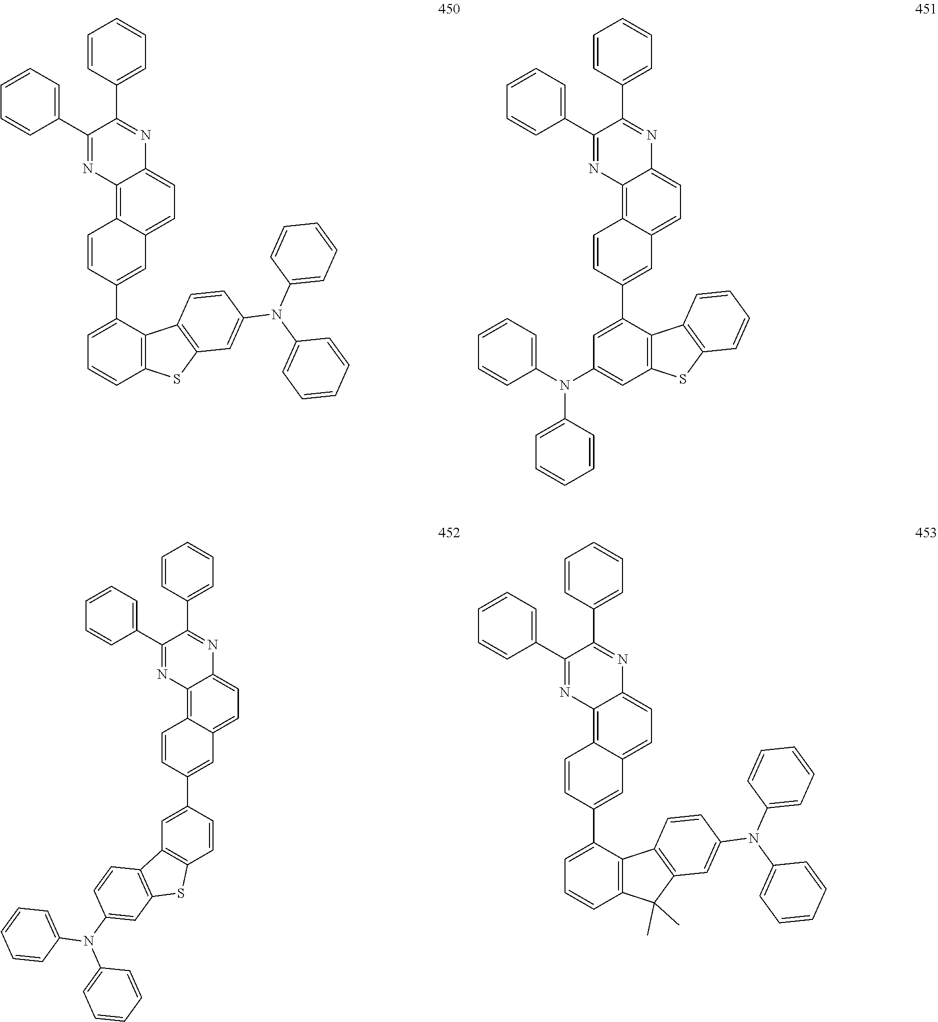 [SMILES: c1ccc(-c2nc3ccc4cc(-c5cccc6sc7cc(N(c8ccccc8)c8ccccc8)ccc7c56)ccc4c3nc2-c2ccccc2)cc1 ; c1ccc(-c2nc3ccc4cc(-c5cc(N(c6ccccc6)c6ccccc6)cc6sc7ccccc7c56)ccc4c3nc2-c2ccccc2)cc1 ; c1ccc(-c2nc3ccc4cc(-c5ccc6sc7cc(N(c8ccccc8)c8ccccc8)ccc7c6c5)ccc4c3nc2-c2ccccc2)cc1 ; CC1(C)c2cc(N(c3ccccc3)c3ccccc3)ccc2-c2c(-c3ccc4c(ccc5nc(-c6ccccc6)c(-c6ccccc6)nc54)c3)cccc21]

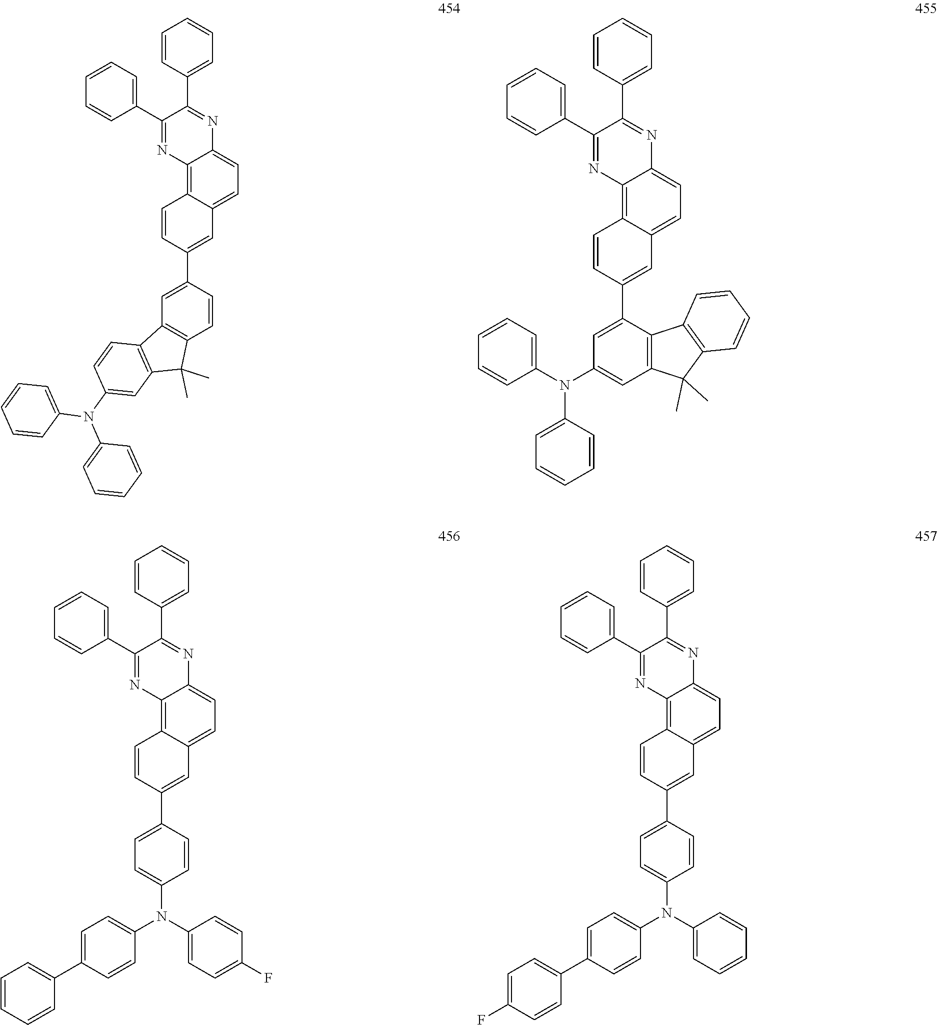 [SMILES: CC1(C)c2ccc(-c3ccc4c(ccc5nc(-c6ccccc6)c(-c6ccccc6)nc54)c3)cc2-c2ccc(N(c3ccccc3)c3ccccc3)cc21 ; CC1(C)c2ccccc2-c2c(-c3ccc4c(ccc5nc(-c6ccccc6)c(-c6ccccc6)nc54)c3)cc(N(c3ccccc3)c3ccccc3)cc21 ; Fc1ccc(N(c2ccc(-c3ccccc3)cc2)c2ccc(-c3ccc4c(ccc5nc(-c6ccccc6)c(-c6ccccc6)nc54)c3)cc2)cc1 ; Fc1ccc(-c2ccc(N(c3ccccc3)c3ccc(-c4ccc5c(ccc6nc(-c7ccccc7)c(-c7ccccc7)nc65)c4)cc3)cc2)cc1]

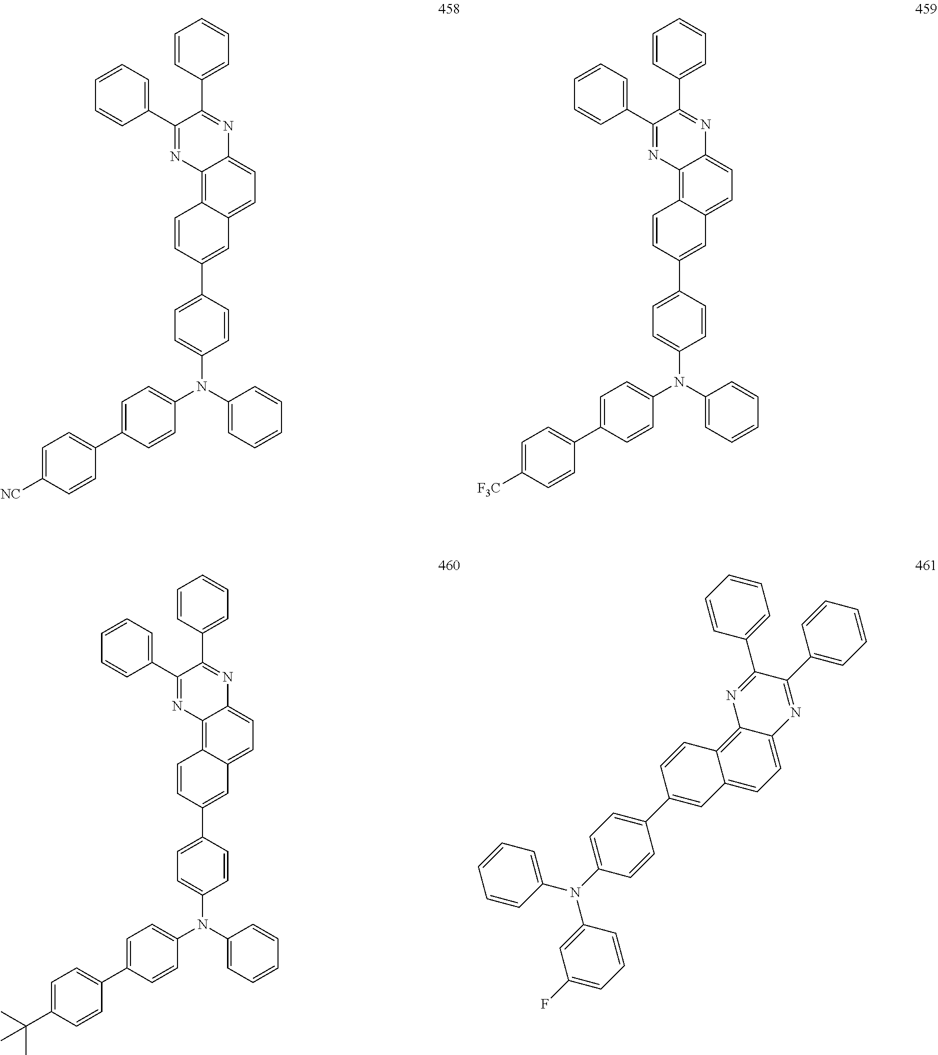 [SMILES: N#Cc1ccc(-c2ccc(N(c3ccccc3)c3ccc(-c4ccc5c(ccc6nc(-c7ccccc7)c(-c7ccccc7)nc65)c4)cc3)cc2)cc1 ; FC(F)(F)c1ccc(-c2ccc(N(c3ccccc3)c3ccc(-c4ccc5c(ccc6nc(-c7ccccc7)c(-c7ccccc7)nc65)c4)cc3)cc2)cc1 ; CC(C)(C)c1ccc(-c2ccc(N(c3ccccc3)c3ccc(-c4ccc5c(ccc6nc(-c7ccccc7)c(-c7ccccc7)nc65)c4)cc3)cc2)cc1 ; Fc1cccc(N(c2ccccc2)c2ccc(-c3ccc4c(ccc5nc(-c6ccccc6)c(-c6ccccc6)nc54)c3)cc2)c1]

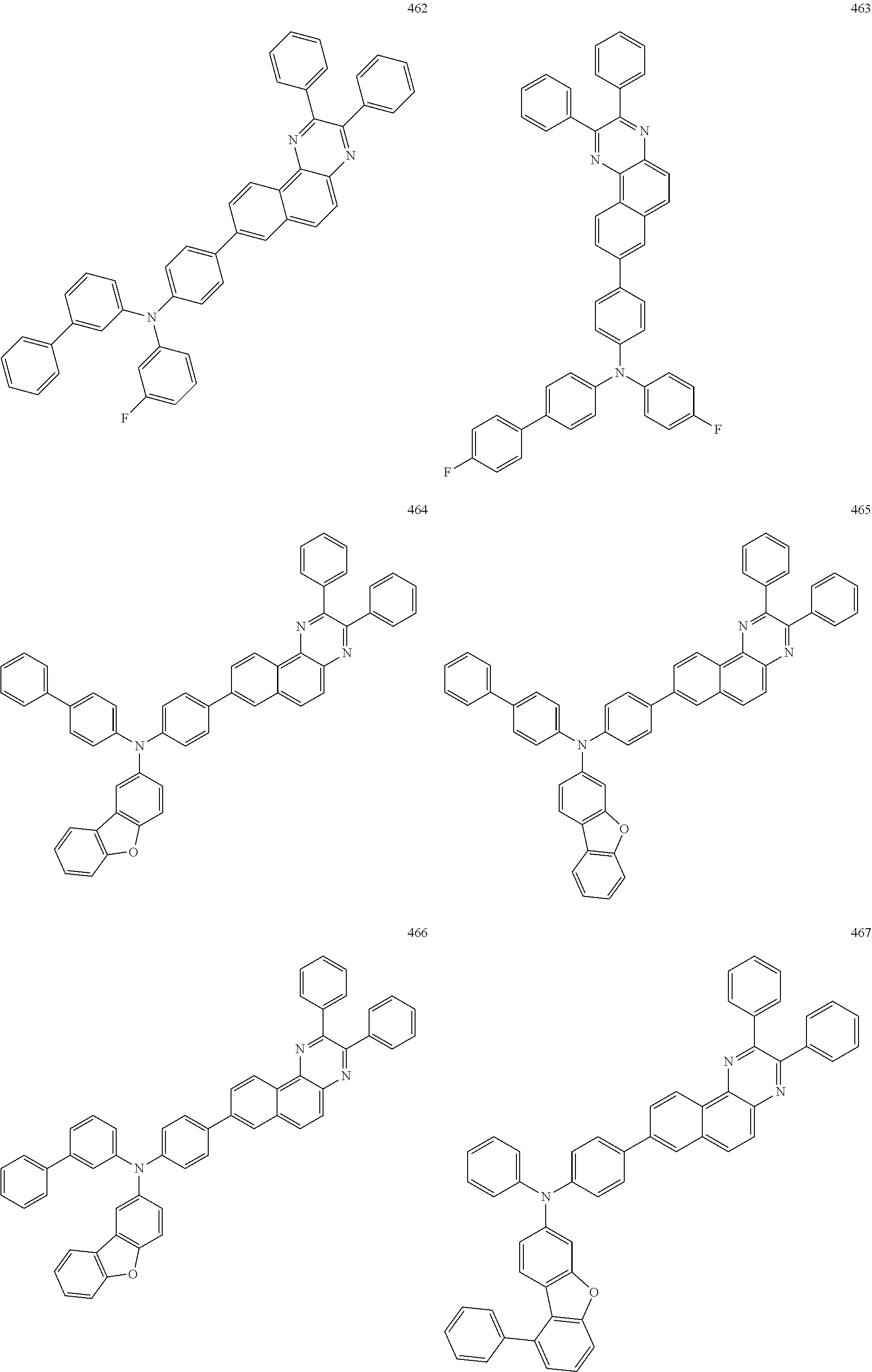

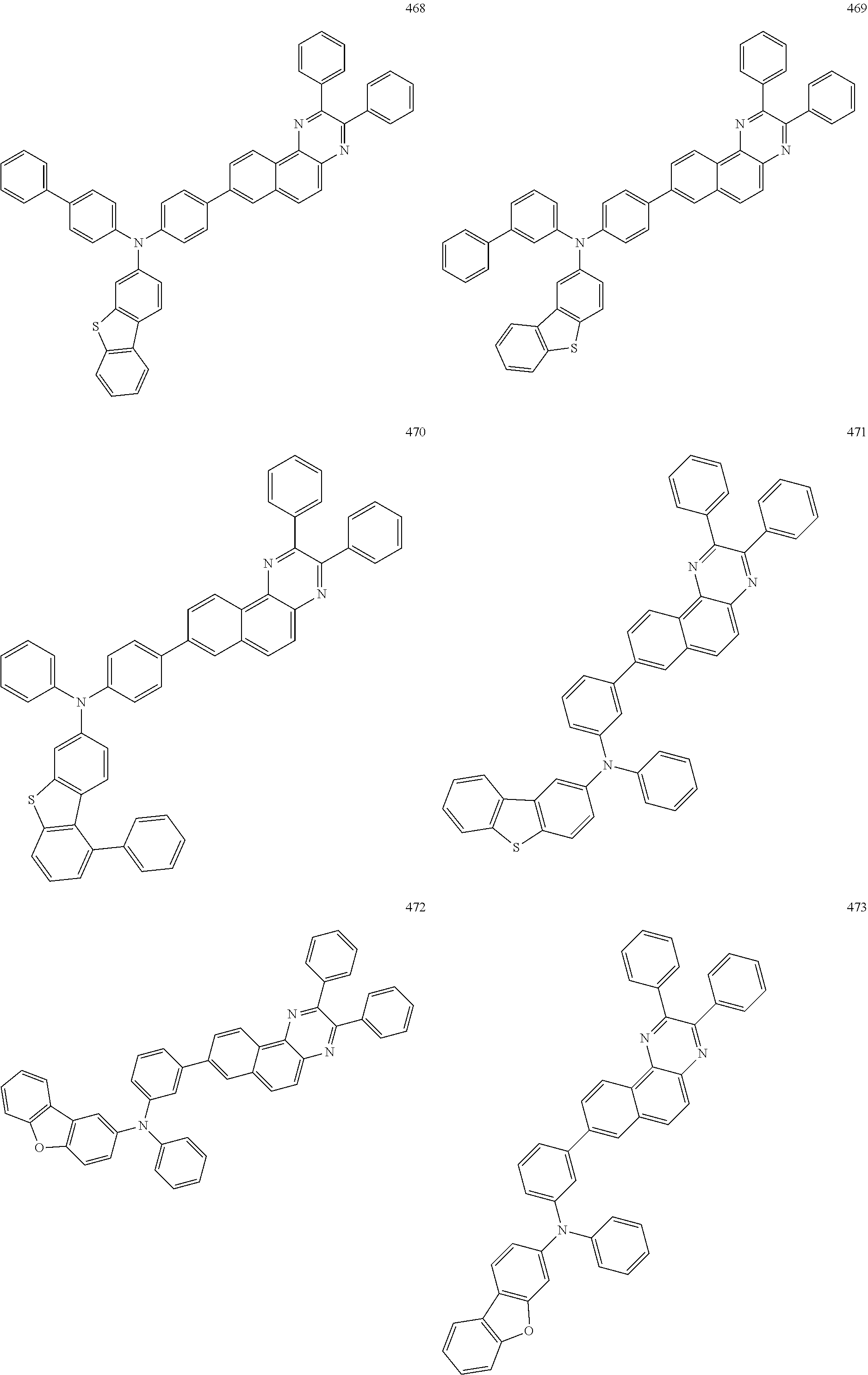

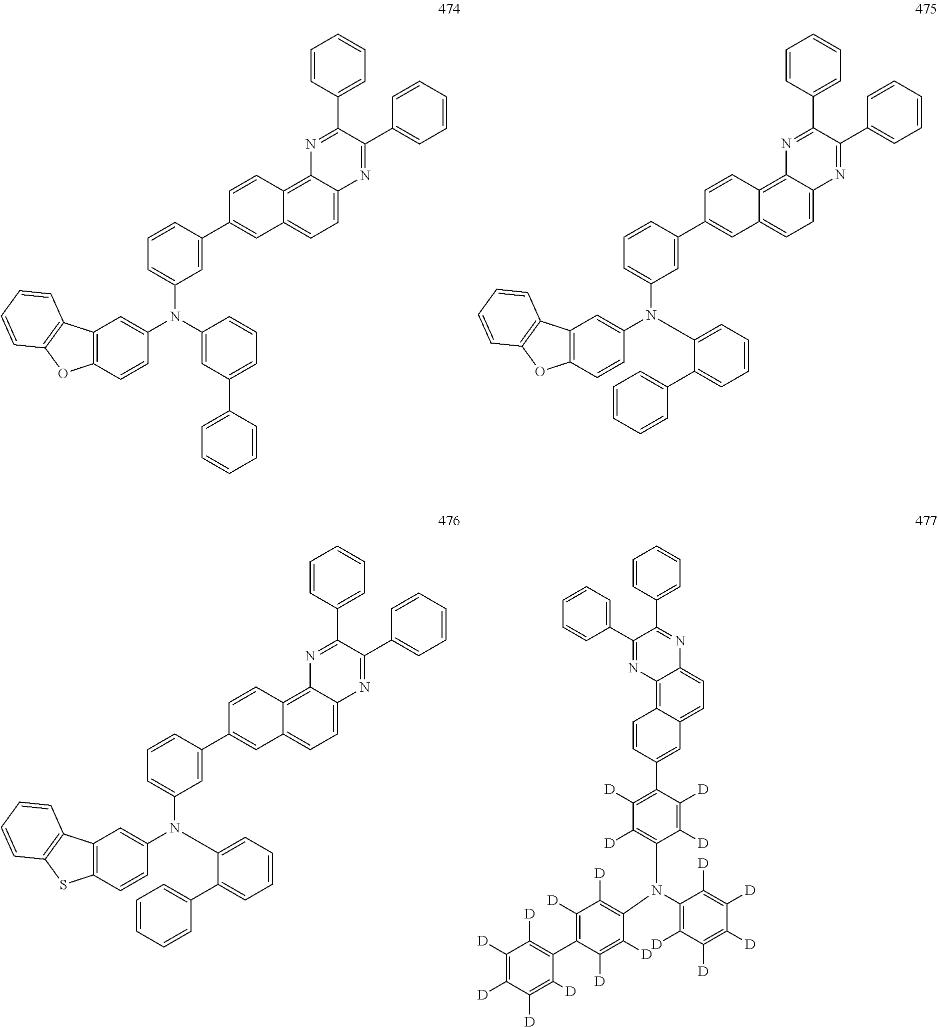 [SMILES: c1ccc(-c2cccc(N(c3cccc(-c4ccc5c(ccc6nc(-c7ccccc7)c(-c7ccccc7)nc65)c4)c3)c3ccc4oc5ccccc5c4c3)c2)cc1 ; c1ccc(-c2ccccc2N(c2cccc(-c3ccc4c(ccc5nc(-c6ccccc6)c(-c6ccccc6)nc54)c3)c2)c2ccc3oc4ccccc4c3c2)cc1 ; c1ccc(-c2ccccc2N(c2cccc(-c3ccc4c(ccc5nc(-c6ccccc6)c(-c6ccccc6)nc54)c3)c2)c2ccc3sc4ccccc4c3c2)cc1 ; [2H]c1c([2H])c([2H])c(-c2c([2H])c([2H])c(N(c3c([2H])c([2H])c([2H])c([2H])c3[2H])c3c([2H])c([2H])c(-c4ccc5c(ccc6nc(-c7ccccc7)c(-c7ccccc7)nc65)c4)c([2H])c3[2H])c([2H])c2[2H])c([2H])c1[2H]]

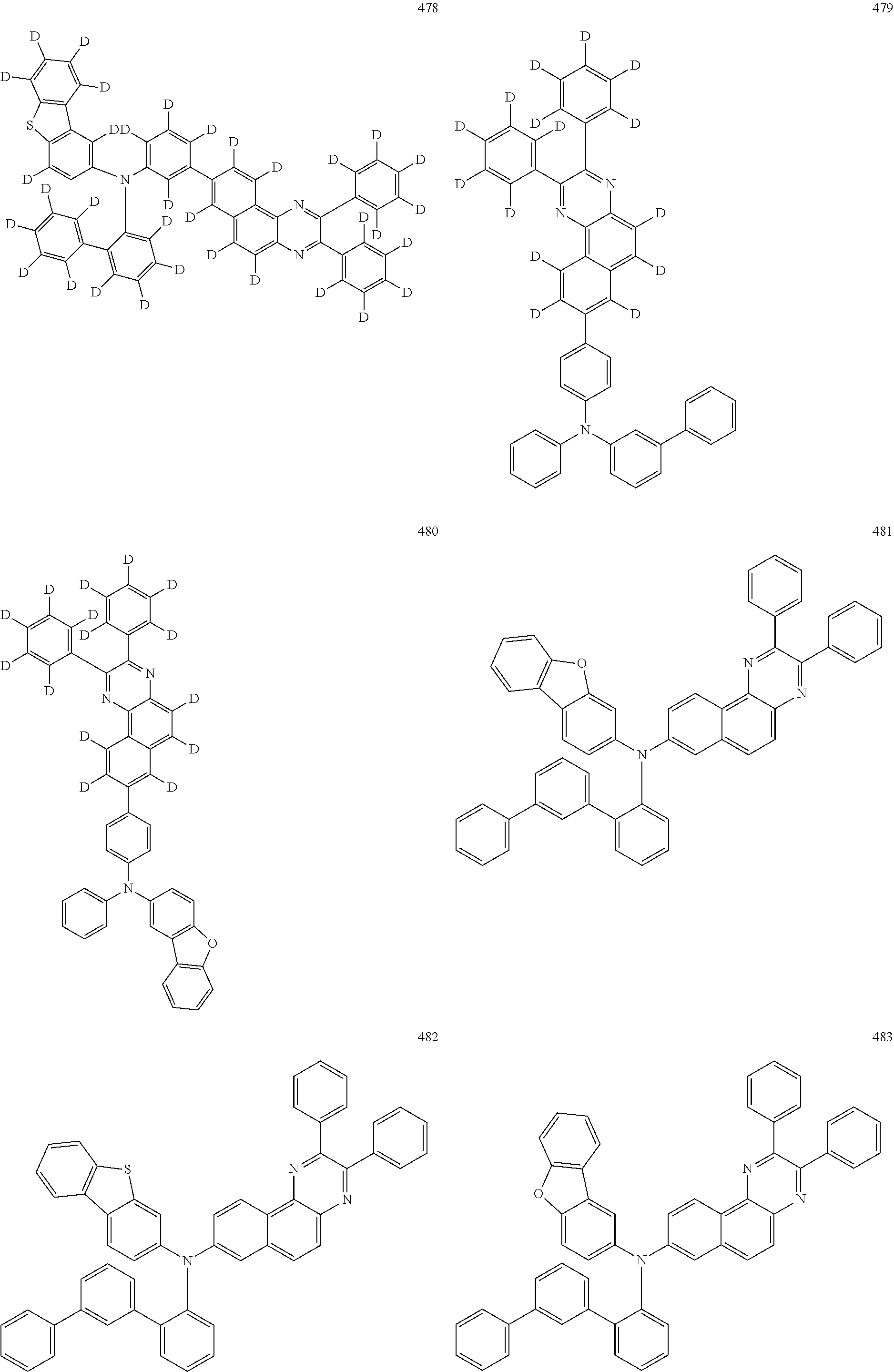

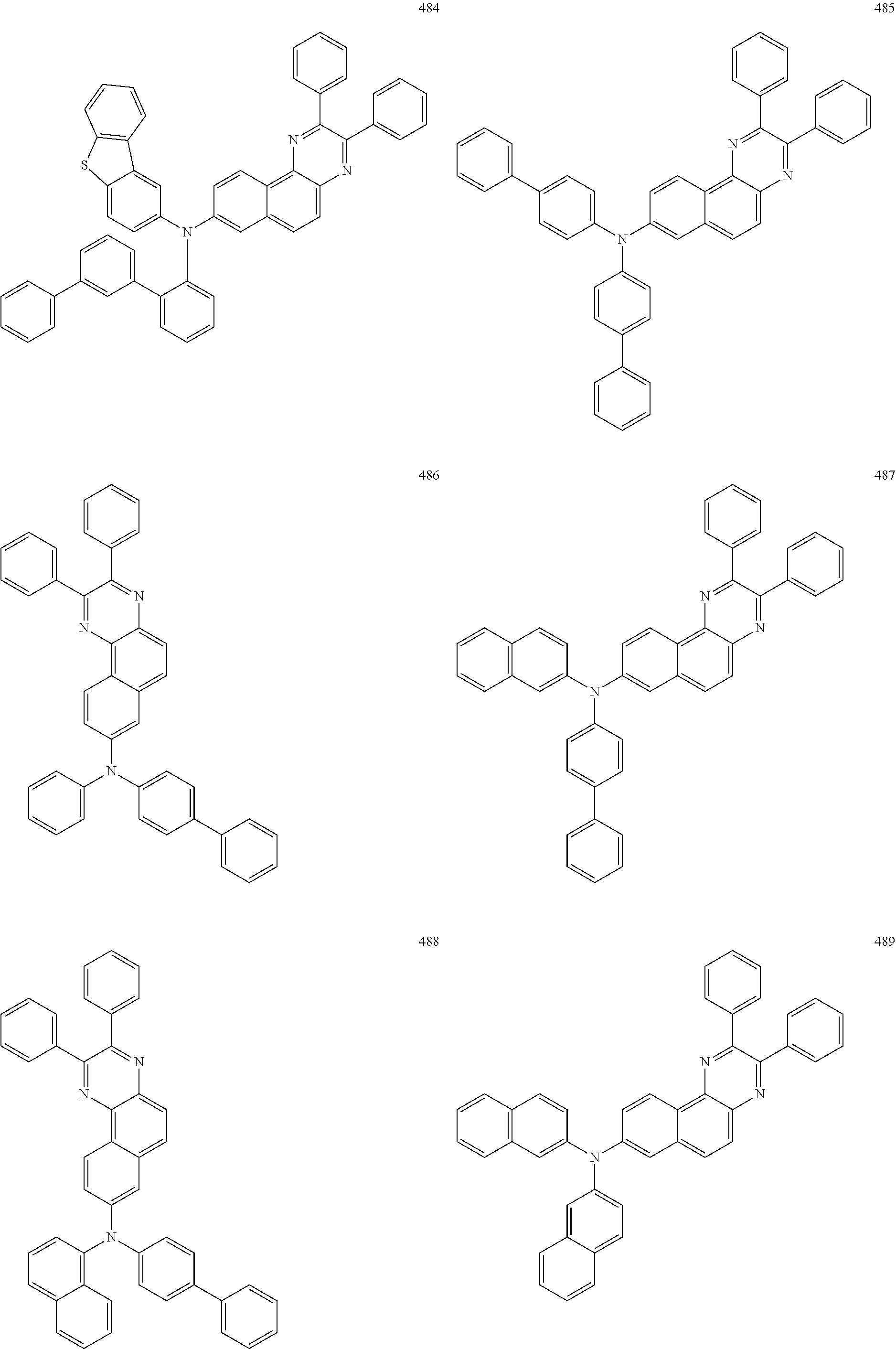

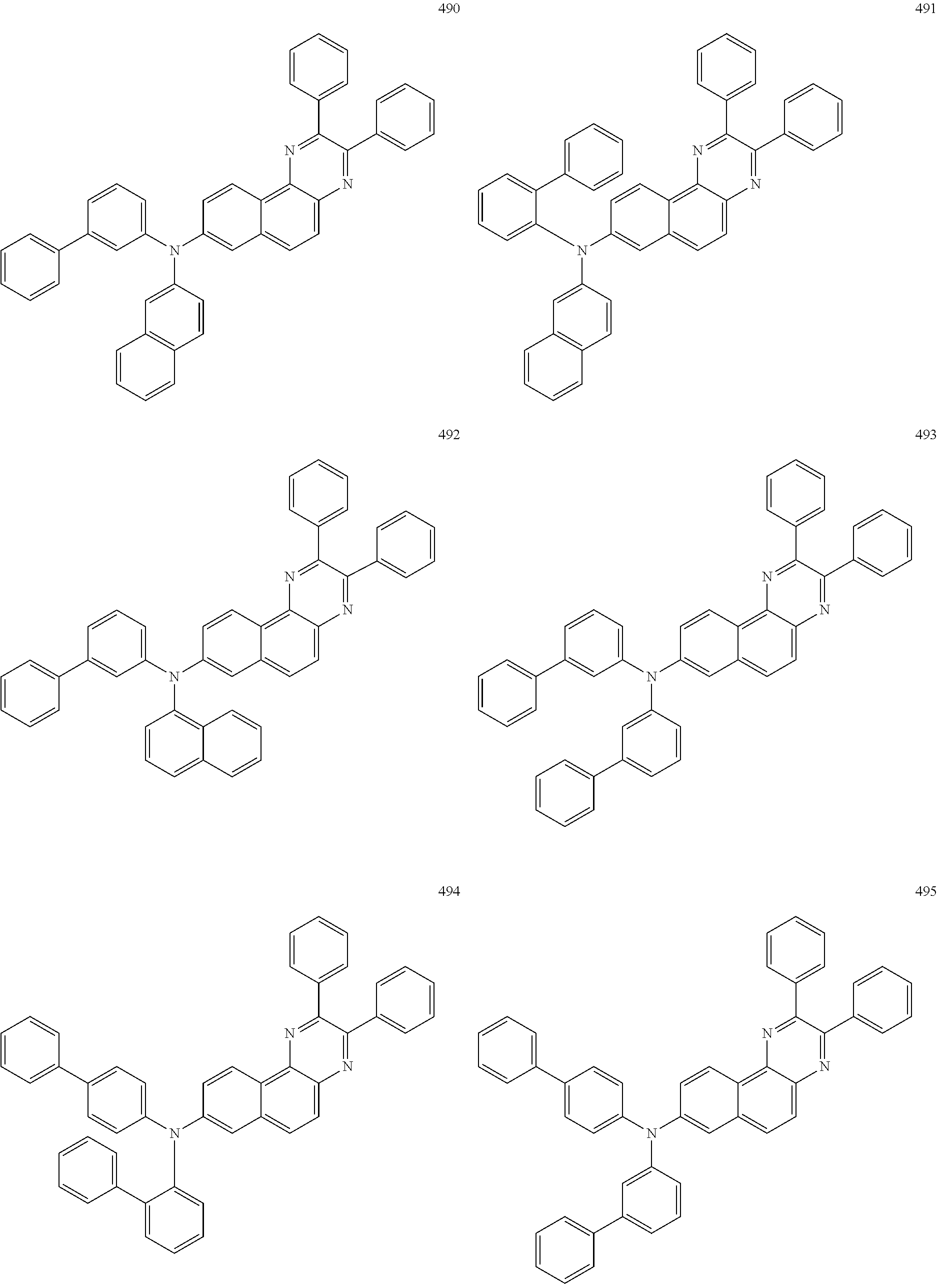

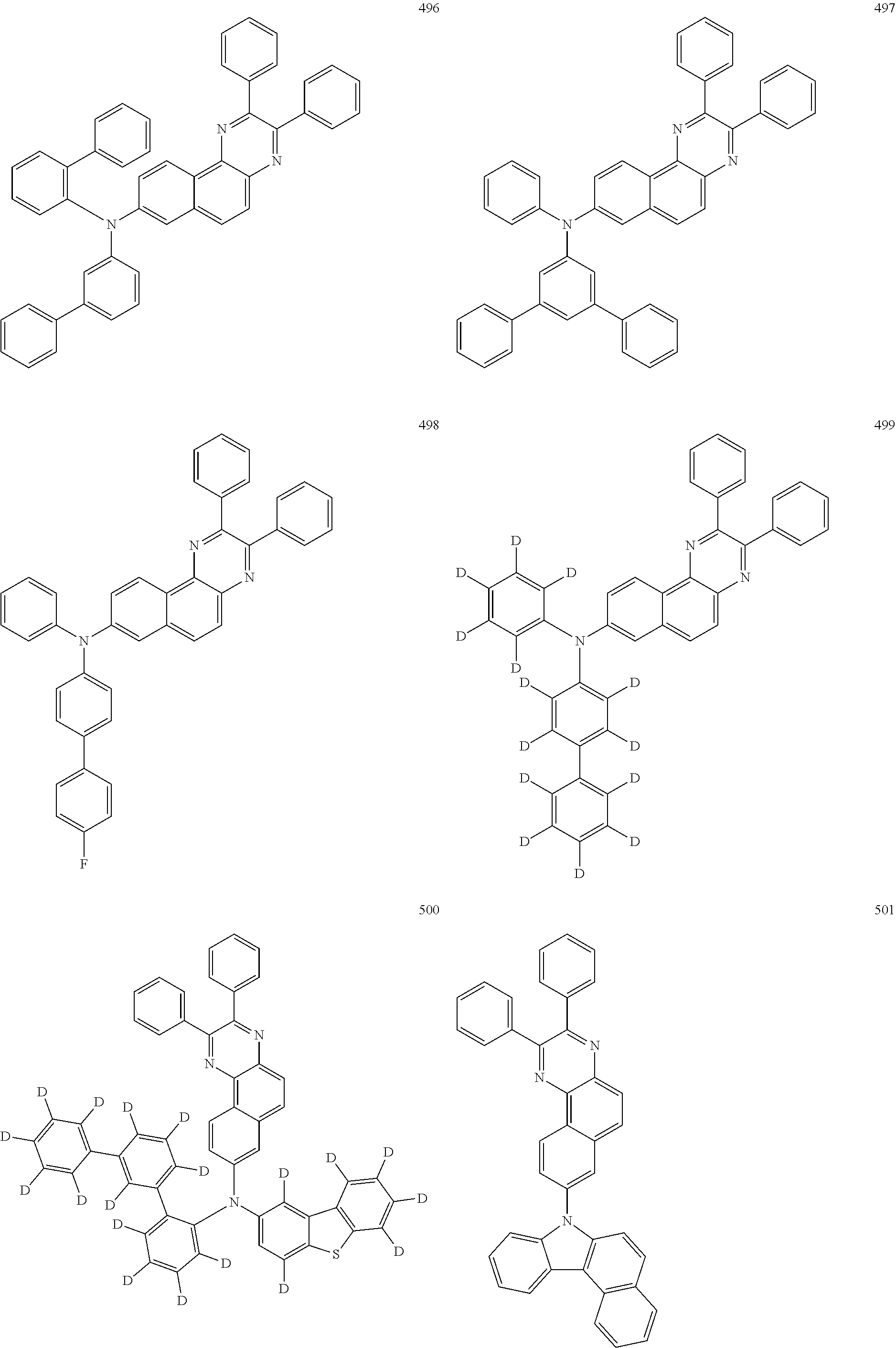

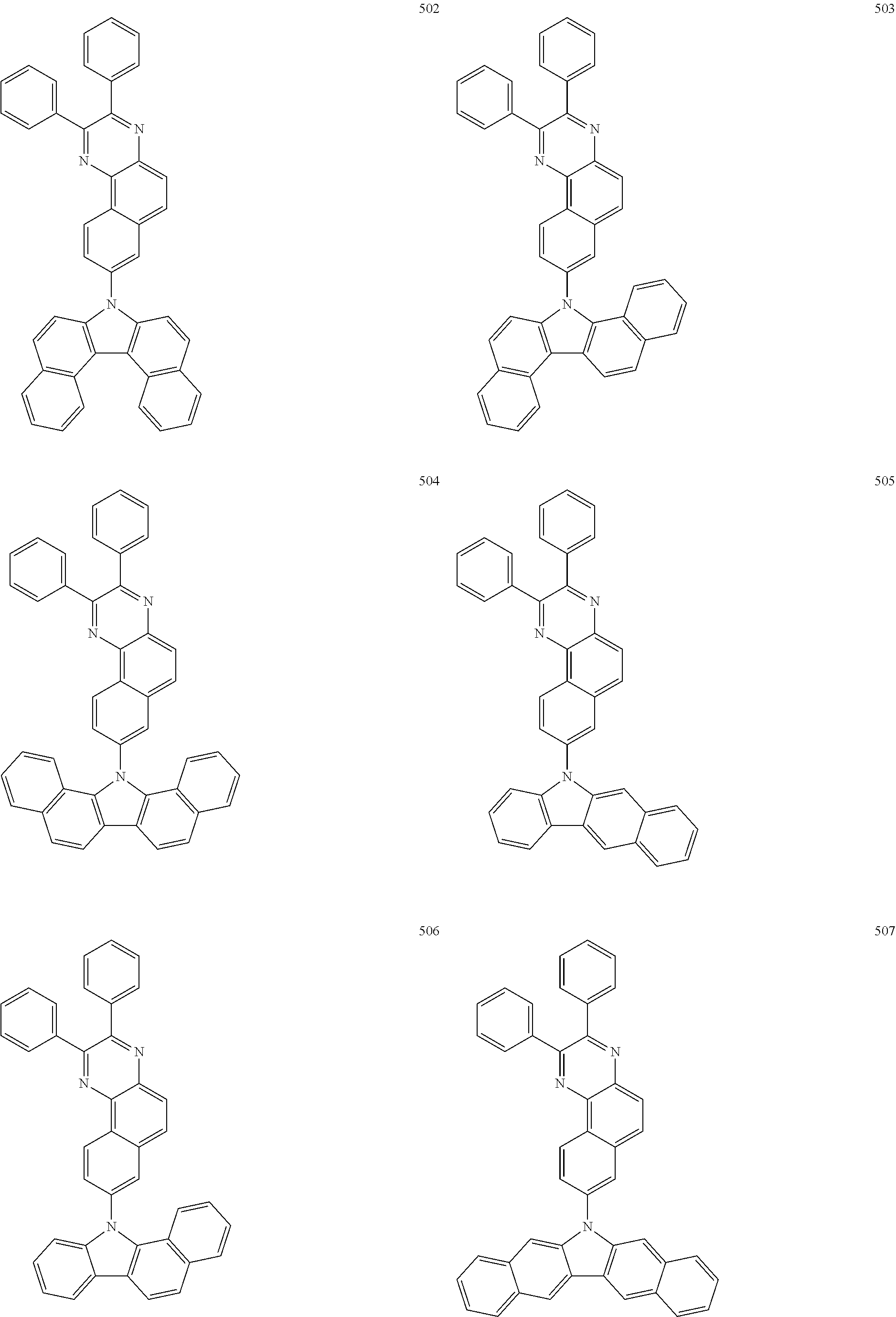 [SMILES: c1ccc(-c2nc3ccc4cc(-n5c6ccc7ccccc7c6c6c7ccccc7ccc65)ccc4c3nc2-c2ccccc2)cc1 ; c1ccc(-c2nc3ccc4cc(-n5c6ccc7ccccc7c6c6ccc7ccccc7c65)ccc4c3nc2-c2ccccc2)cc1 ; c1ccc(-c2nc3ccc4cc(-n5c6c7ccccc7ccc6c6ccc7ccccc7c65)ccc4c3nc2-c2ccccc2)cc1 ; c1ccc(-c2nc3ccc4cc(-n5c6ccccc6c6cc7ccccc7cc65)ccc4c3nc2-c2ccccc2)cc1 ; c1ccc(-c2nc3ccc4cc(-n5c6ccccc6c6ccc7ccccc7c65)ccc4c3nc2-c2ccccc2)cc1 ; c1ccc(-c2nc3ccc4cc(-n5c6cc7ccccc7cc6c6cc7ccccc7cc65)ccc4c3nc2-c2ccccc2)cc1]

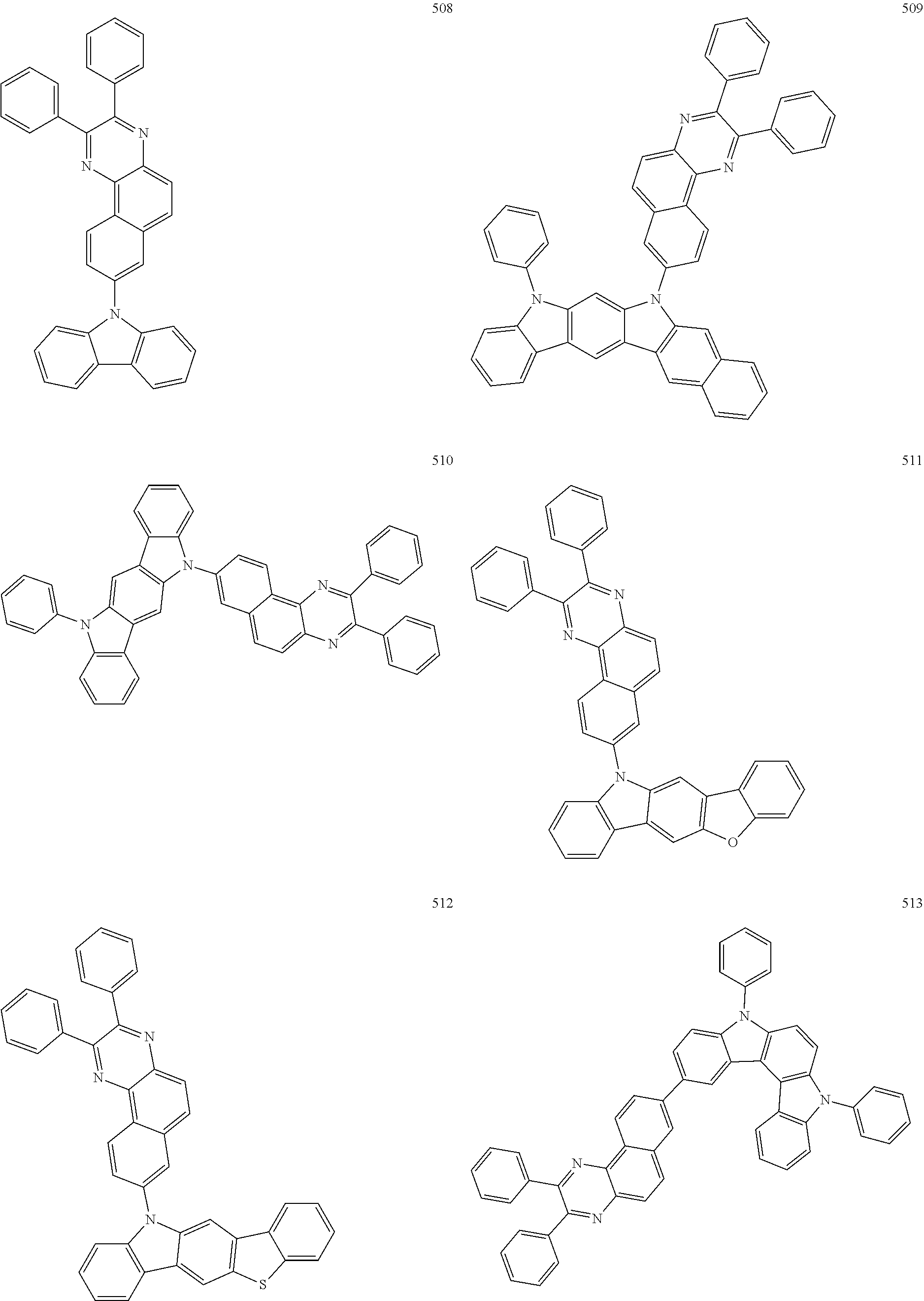 [SMILES: c1ccc(-c2nc3ccc4cc(-n5c6ccccc6c6ccccc65)ccc4c3nc2-c2ccccc2)cc1 ; c1ccc(-c2nc3ccc4cc(-n5c6cc7ccccc7cc6c6cc7c8ccccc8n(-c8ccccc8)c7cc65)ccc4c3nc2-c2ccccc2)cc1 ; c1ccc(-c2nc3ccc4cc(-n5c6ccccc6c6cc7c(cc65)c5ccccc5n7-c5ccccc5)ccc4c3nc2-c2ccccc2)cc1 ; c1ccc(-c2nc3ccc4cc(-n5c6ccccc6c6cc7oc8ccccc8c7cc65)ccc4c3nc2-c2ccccc2)cc1 ; c1ccc(-c2nc3ccc4cc(-n5c6ccccc6c6cc7sc8ccccc8c7cc65)ccc4c3nc2-c2ccccc2)cc1 ; c1ccc(-c2nc3ccc4cc(-c5ccc6c(c5)c5c7c8ccccc8n(-c8ccccc8)c7ccc5n6-c5ccccc5)ccc4c3nc2-c2ccccc2)cc1]

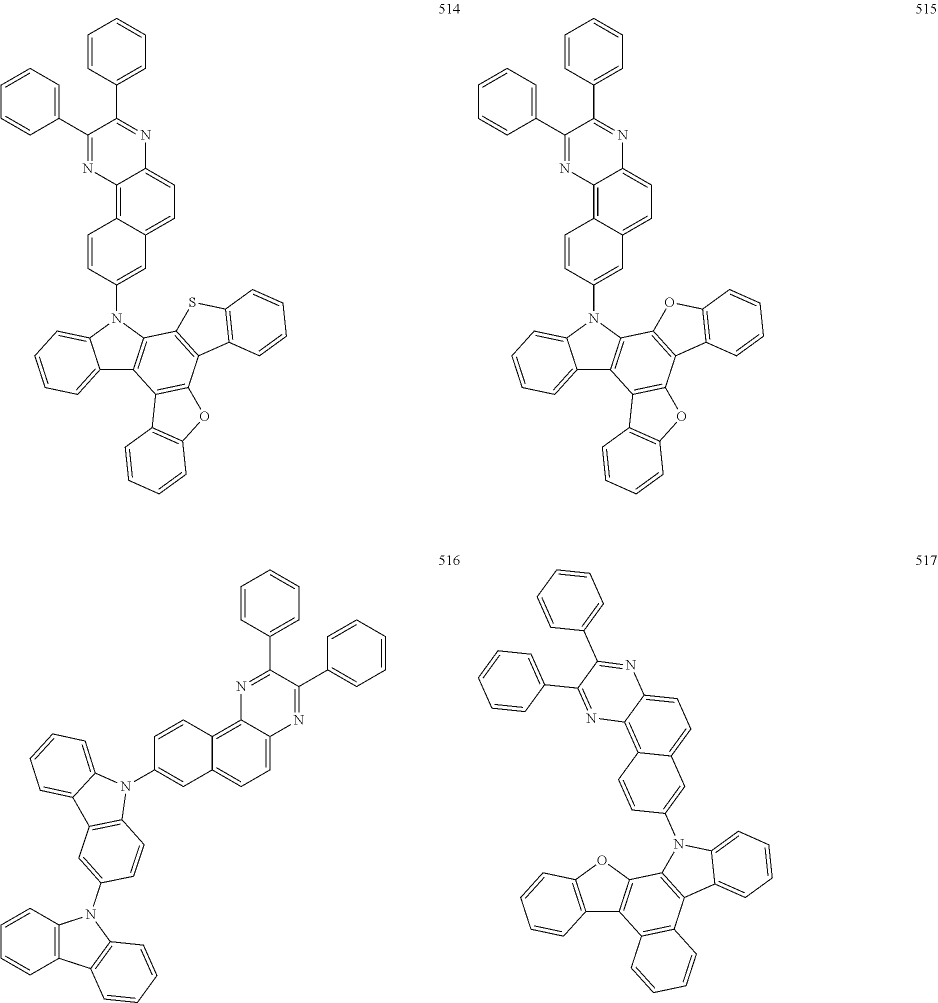 [SMILES: c1ccc(-c2nc3ccc4cc(-n5c6ccccc6c6c7c8ccccc8oc7c7c8ccccc8sc7c65)ccc4c3nc2-c2ccccc2)cc1 ; c1ccc(-c2nc3ccc4cc(-n5c6ccccc6c6c7c8ccccc8oc7c7c8ccccc8oc7c65)ccc4c3nc2-c2ccccc2)cc1 ; c1ccc(-c2nc3ccc4cc(-n5c6ccccc6c6cc(-n7c8ccccc8c8ccccc87)ccc65)ccc4c3nc2-c2ccccc2)cc1 ; c1ccc(-c2nc3ccc4cc(-n5c6ccccc6c6c7ccccc7c7c8ccccc8oc7c65)ccc4c3nc2-c2ccccc2)cc1]

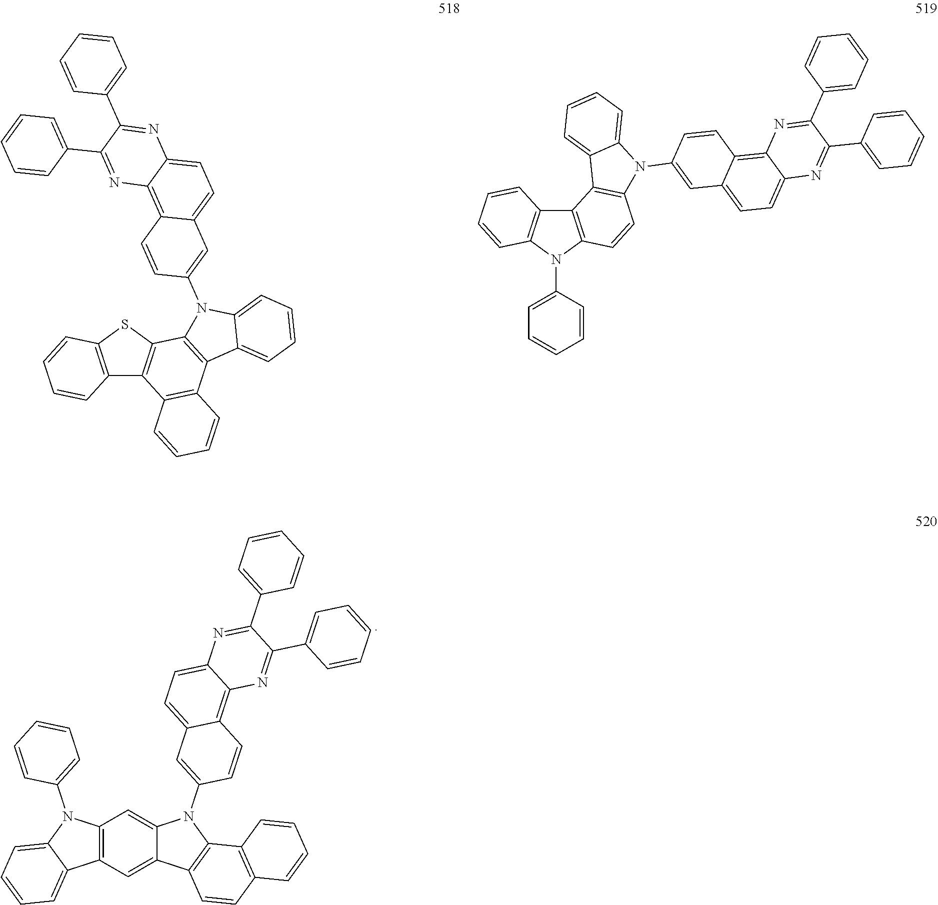 [SMILES: c1ccc(-c2nc3ccc4cc(-n5c6ccccc6c6c7ccccc7c7c8ccccc8sc7c65)ccc4c3nc2-c2ccccc2)cc1 ; c1ccc(-c2nc3ccc4cc(-n5c6ccccc6c6c7c8ccccc8n(-c8ccccc8)c7ccc65)ccc4c3nc2-c2ccccc2)cc1 ; c1ccc(-c2nc3ccc4cc(-n5c6cc7c(cc6c6ccc8ccccc8c65)c5ccccc5n7-c5ccccc5)ccc4c3nc2-c2ccccc2)cc1]

10. An organic light emitting device comprising:
a first electrode;
a second electrode provided opposite to the first electrode; and
one or more organic material layers provided between the first electrode and the second electrode,
wherein one or more layers of the organic material layers include the heterocyclic compound of claim 1.

11. The organic light emitting device of claim 10, wherein the organic material layer includes a light emitting layer, and the light emitting layer includes the heterocyclic compound.

12. The organic light emitting device of claim 10, wherein the organic material layer includes a light emitting layer, the light emitting layer includes a host material, and the host material includes the heterocyclic compound.

13. The organic light emitting device of claim 10, further comprising one, two or more layers selected from the group consisting of a light emitting layer, a hole injection layer, a hole transport layer, an electron injection layer, an electron transport layer, an electron blocking layer and a hole blocking layer.

14. The organic light emitting device of claim 10, wherein the organic material layer further includes a heterocyclic compound represented by the following Chemical Formula A: 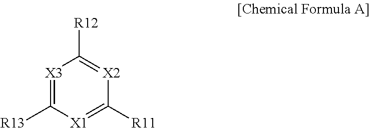
in Chemical Formula A,
X1 to X3 are the same as or different from each other, and each independently N or CR″;
at least two or more of X1 to X3 are N;
R11 to R13 and R″ are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heteroring; and
at least one of R11 to R13 is a group represented by the following Chemical Formula B-1 or Chemical Formula B-2, 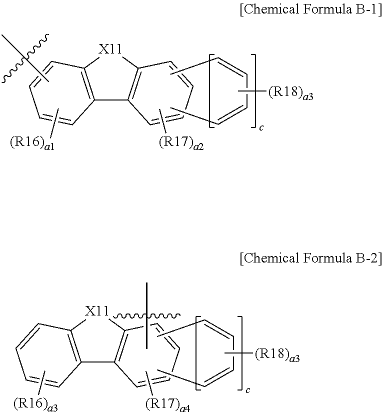
in Chemical Formulae B-1 and B-2,
X11 is O or S;
c is an integer of 0 or 1;
R16 to R18 are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group;
a1 is one of integers of 1 to 3;
a2 is one of integers of 1 to 4, and when c is 1, a2 is 1 or 2;
a3 is one of integers of 1 to 4; and
a4 is one of integers of 1 to 3, and when c is 1, a4 is 1.

15. The organic light emitting device of claim 14, wherein Chemical Formula A is represented by any one of the following Chemical Formula A-1, Chemical Formula A-2 and Chemical Formula A-3: 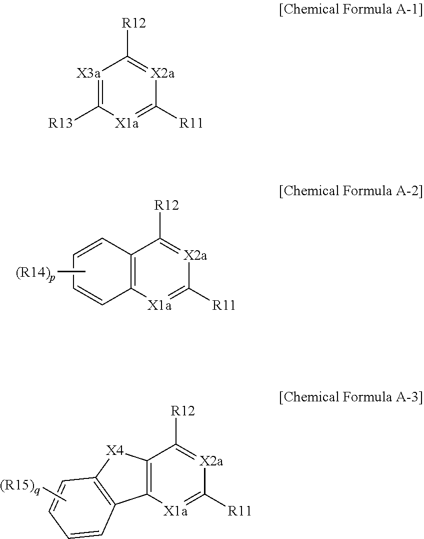
in Chemical Formula A-1 to Chemical Formula A-3,
X1a to X3a have the same definitions as X1 to X3 of Chemical Formula A;
X4 is O or S;
R11 to R15 are the same as or different from each other, and each independently selected from the group consisting of hydrogen; deuterium; a halogen group; a cyano group; a substituted or unsubstituted C1 to C60 alkyl group; a substituted or group; a substituted or unsubstituted C2 to C60 alkenyl unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group;
p is one of integers of 1 to 4; and
q is one of integers of 1 to 4.

16. The organic light emitting device of claim 14, wherein the heterocyclic compound represented by Chemical Formula A is any one selected from the group consisting of the following compounds:

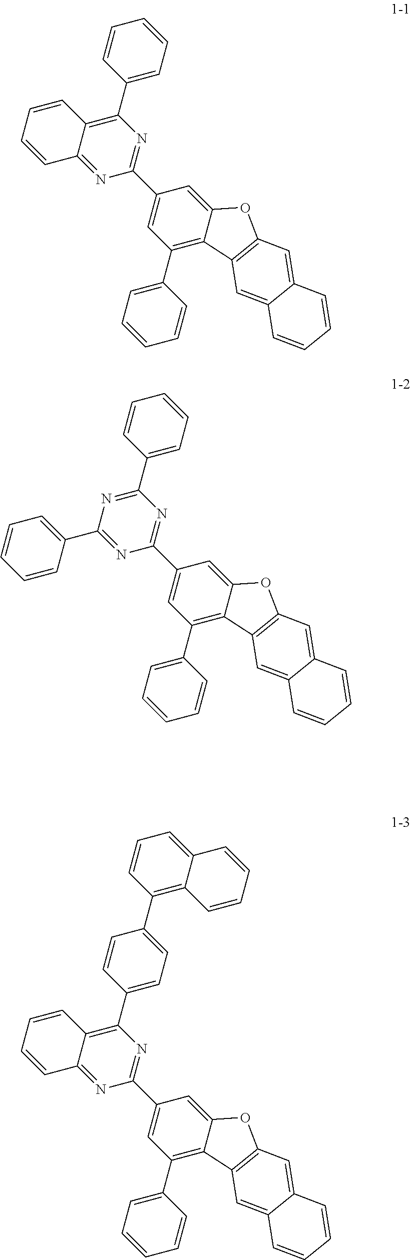 [SMILES: c1ccc(-c2nc(-c3cc(-c4ccccc4)c4c(c3)oc3cc5ccccc5cc34)nc3ccccc23)cc1 ; c1ccc(-c2nc(-c3ccccc3)nc(-c3cc(-c4ccccc4)c4c(c3)oc3cc5ccccc5cc34)n2)cc1 ; c1ccc(-c2cc(-c3nc(-c4ccc(-c5cccc6ccccc56)cc4)c4ccccc4n3)cc3oc4cc5ccccc5cc4c23)cc1]

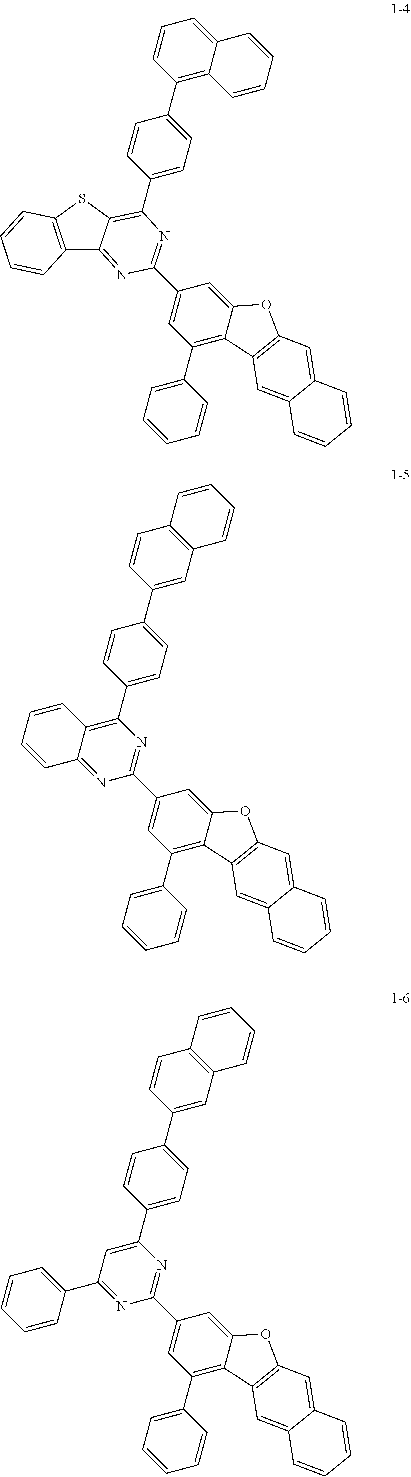 [SMILES: c1ccc(-c2cc(-c3nc(-c4ccc(-c5cccc6ccccc56)cc4)c4sc5ccccc5c4n3)cc3oc4cc5ccccc5cc4c23)cc1 ; c1ccc(-c2cc(-c3nc(-c4ccc(-c5ccc6ccccc6c5)cc4)c4ccccc4n3)cc3oc4cc5ccccc5cc4c23)cc1 ; c1ccc(-c2cc(-c3ccc(-c4ccc5ccccc5c4)cc3)nc(-c3cc(-c4ccccc4)c4c(c3)oc3cc5ccccc5cc34)n2)cc1]

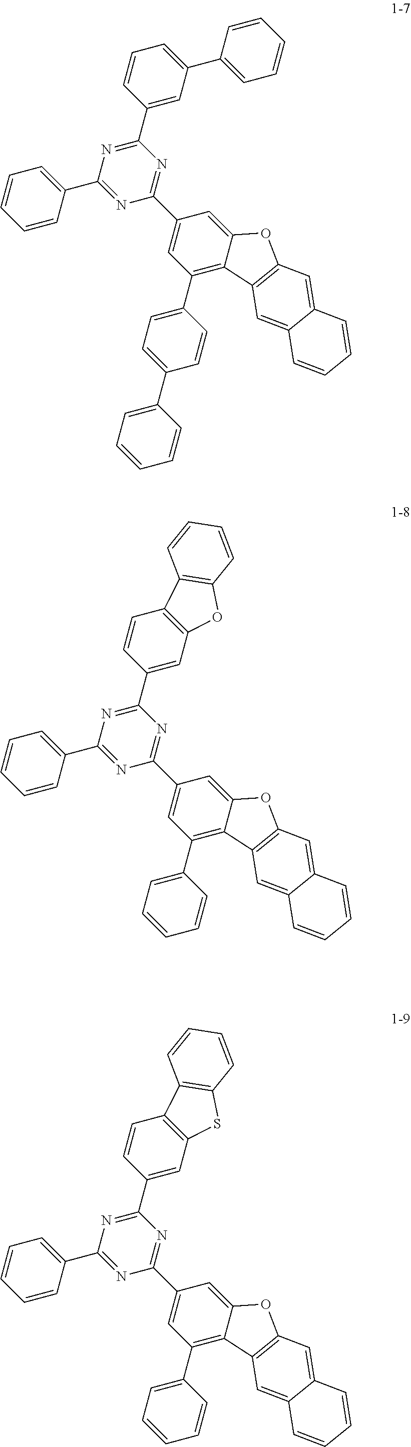 [SMILES: c1ccc(-c2ccc(-c3cc(-c4nc(-c5ccccc5)nc(-c5cccc(-c6ccccc6)c5)n4)cc4oc5cc6ccccc6cc5c34)cc2)cc1 ; c1ccc(-c2nc(-c3ccc4c(c3)oc3ccccc34)nc(-c3cc(-c4ccccc4)c4c(c3)oc3cc5ccccc5cc34)n2)cc1 ; c1ccc(-c2nc(-c3cc(-c4ccccc4)c4c(c3)oc3cc5ccccc5cc34)nc(-c3ccc4c(c3)sc3ccccc34)n2)cc1]

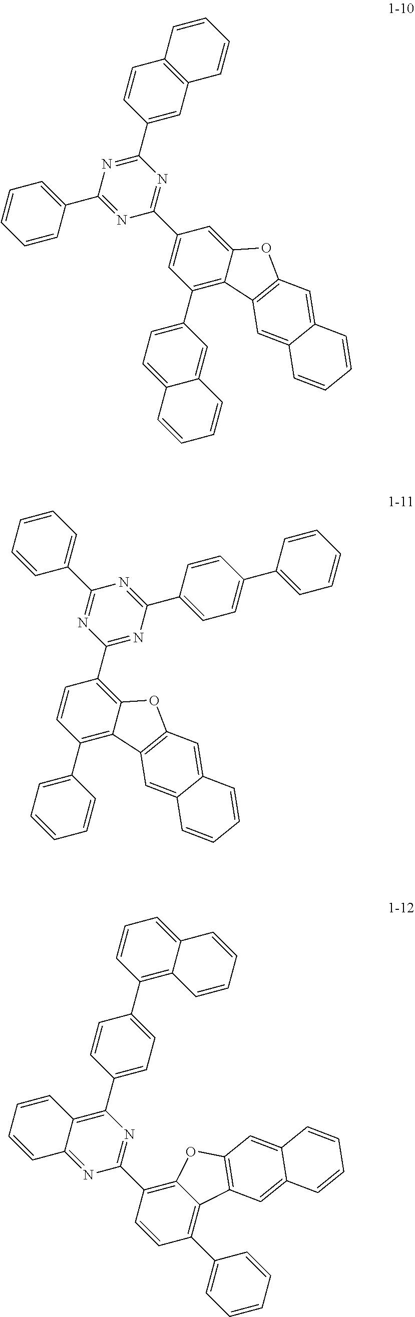 [SMILES: c1ccc(-c2nc(-c3ccc4ccccc4c3)nc(-c3cc(-c4ccc5ccccc5c4)c4c(c3)oc3cc5ccccc5cc34)n2)cc1 ; c1ccc(-c2ccc(-c3nc(-c4ccccc4)nc(-c4ccc(-c5ccccc5)c5c4oc4cc6ccccc6cc45)n3)cc2)cc1 ; c1ccc(-c2ccc(-c3nc(-c4ccc(-c5cccc6ccccc56)cc4)c4ccccc4n3)c3oc4cc5ccccc5cc4c23)cc1]

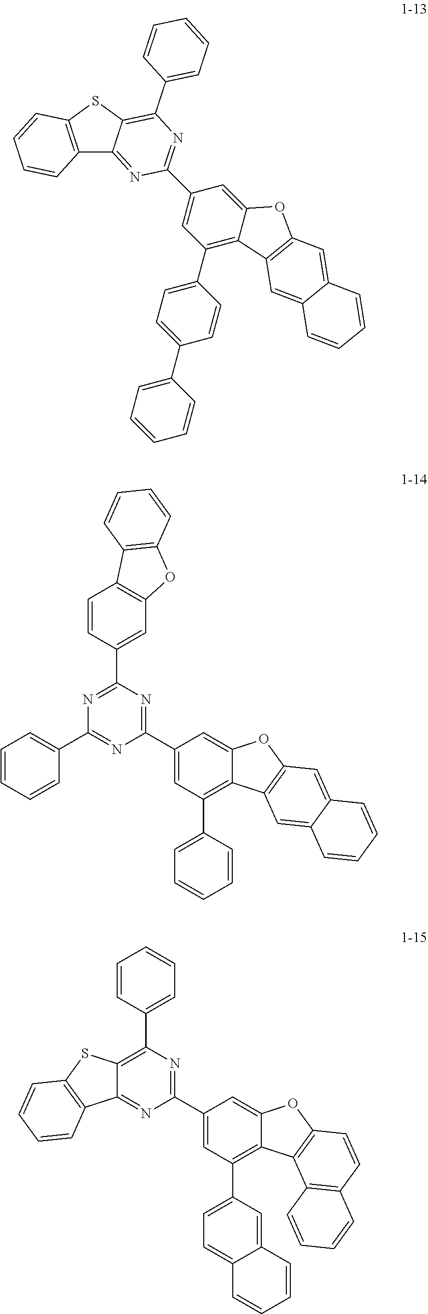 [SMILES: c1ccc(-c2ccc(-c3cc(-c4nc(-c5ccccc5)c5sc6ccccc6c5n4)cc4oc5cc6ccccc6cc5c34)cc2)cc1 ; c1ccc(-c2nc(-c3ccc4c(c3)oc3ccccc34)nc(-c3cc(-c4ccccc4)c4c(c3)oc3cc5ccccc5cc34)n2)cc1 ; c1ccc(-c2nc(-c3cc(-c4ccc5ccccc5c4)c4c(c3)oc3ccc5ccccc5c34)nc3c2sc2ccccc23)cc1]

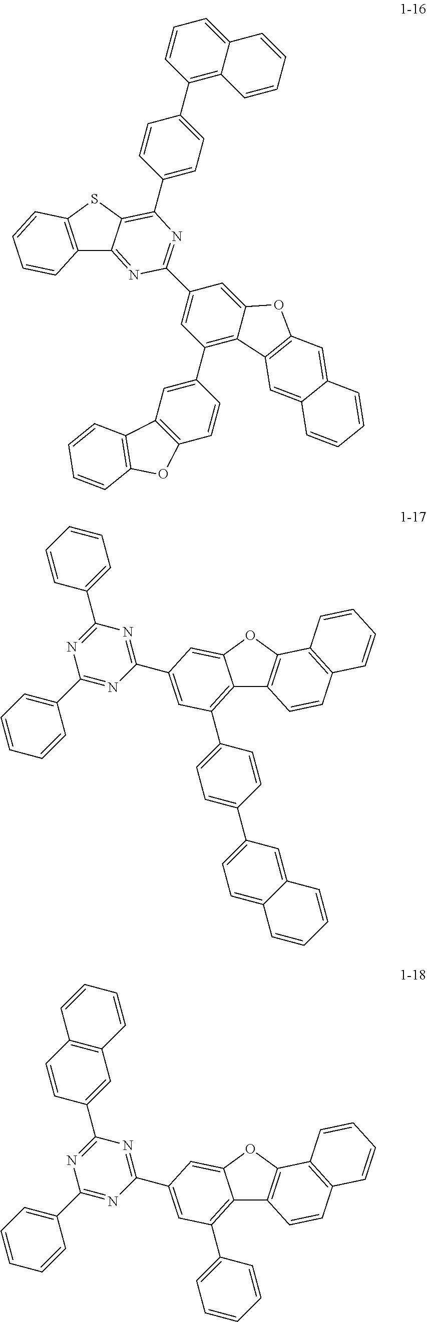

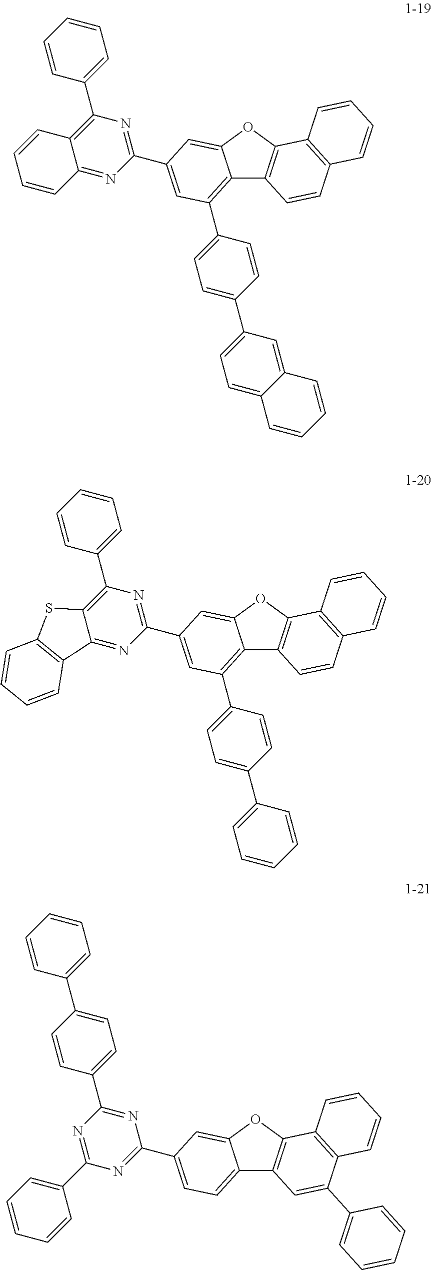 [SMILES: c1ccc(-c2nc(-c3cc(-c4ccc(-c5ccc6ccccc6c5)cc4)c4c(c3)oc3c5ccccc5ccc34)nc3ccccc23)cc1 ; c1ccc(-c2ccc(-c3cc(-c4nc(-c5ccccc5)c5sc6ccccc6c5n4)cc4oc5c6ccccc6ccc5c34)cc2)cc1 ; c1ccc(-c2ccc(-c3nc(-c4ccccc4)nc(-c4ccc5c(c4)oc4c6ccccc6c(-c6ccccc6)cc54)n3)cc2)cc1]

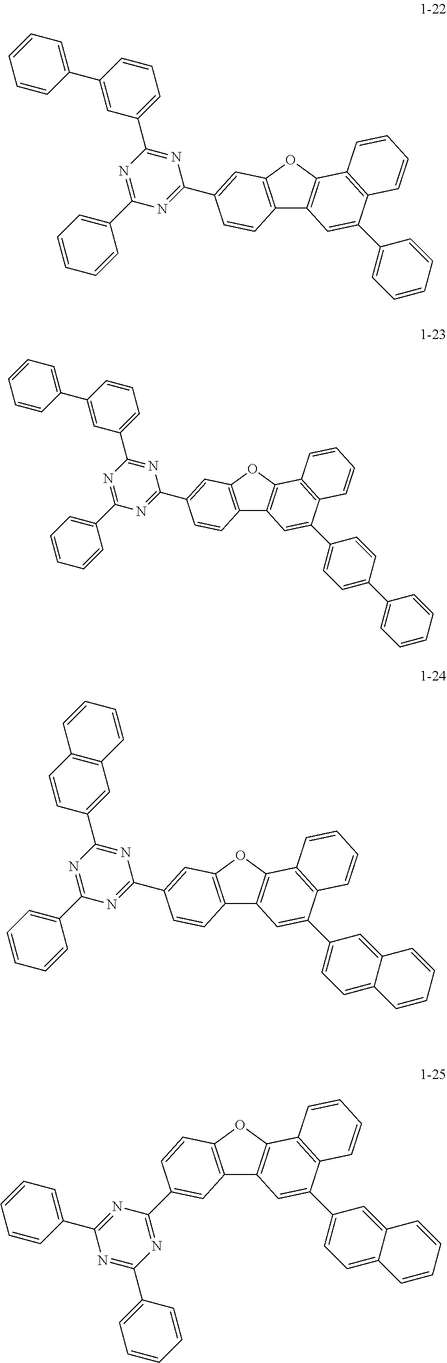 [SMILES: c1ccc(-c2cccc(-c3nc(-c4ccccc4)nc(-c4ccc5c(c4)oc4c6ccccc6c(-c6ccccc6)cc54)n3)c2)cc1 ; c1ccc(-c2ccc(-c3cc4c5ccc(-c6nc(-c7ccccc7)nc(-c7cccc(-c8ccccc8)c7)n6)cc5oc4c4ccccc34)cc2)cc1 ; c1ccc(-c2nc(-c3ccc4ccccc4c3)nc(-c3ccc4c(c3)oc3c5ccccc5c(-c5ccc6ccccc6c5)cc43)n2)cc1 ; c1ccc(-c2nc(-c3ccccc3)nc(-c3ccc4oc5c6ccccc6c(-c6ccc7ccccc7c6)cc5c4c3)n2)cc1]

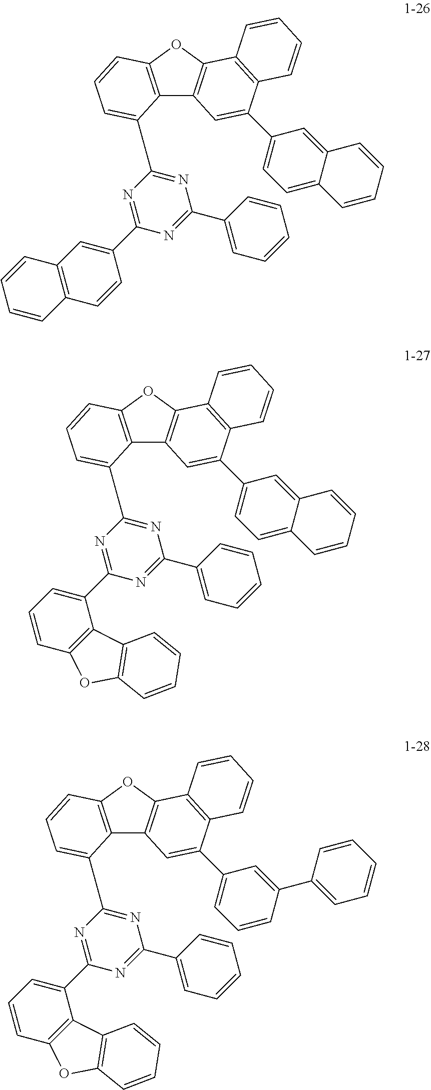 [SMILES: c1ccc(-c2nc(-c3ccc4ccccc4c3)nc(-c3cccc4oc5c6ccccc6c(-c6ccc7ccccc7c6)cc5c34)n2)cc1 ; c1ccc(-c2nc(-c3cccc4oc5ccccc5c34)nc(-c3cccc4oc5c6ccccc6c(-c6ccc7ccccc7c6)cc5c34)n2)cc1 ; c1ccc(-c2cccc(-c3cc4c(oc5cccc(-c6nc(-c7ccccc7)nc(-c7cccc8oc9ccccc9c78)n6)c54)c4ccccc34)c2)cc1]

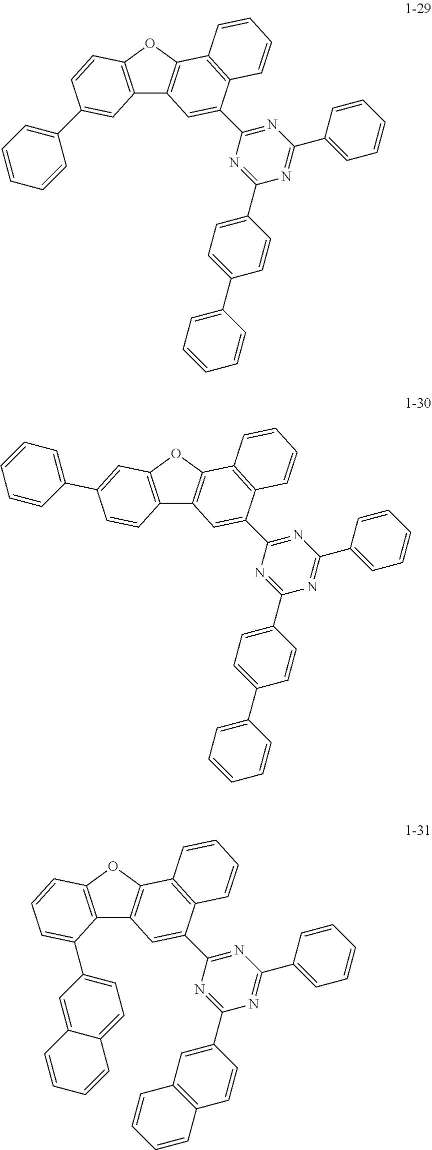 [SMILES: c1ccc(-c2ccc(-c3nc(-c4ccccc4)nc(-c4cc5c6cc(-c7ccccc7)ccc6oc5c5ccccc45)n3)cc2)cc1 ; c1ccc(-c2ccc(-c3nc(-c4ccccc4)nc(-c4cc5c6ccc(-c7ccccc7)cc6oc5c5ccccc45)n3)cc2)cc1 ; c1ccc(-c2nc(-c3ccc4ccccc4c3)nc(-c3cc4c(oc5cccc(-c6ccc7ccccc7c6)c54)c4ccccc34)n2)cc1]

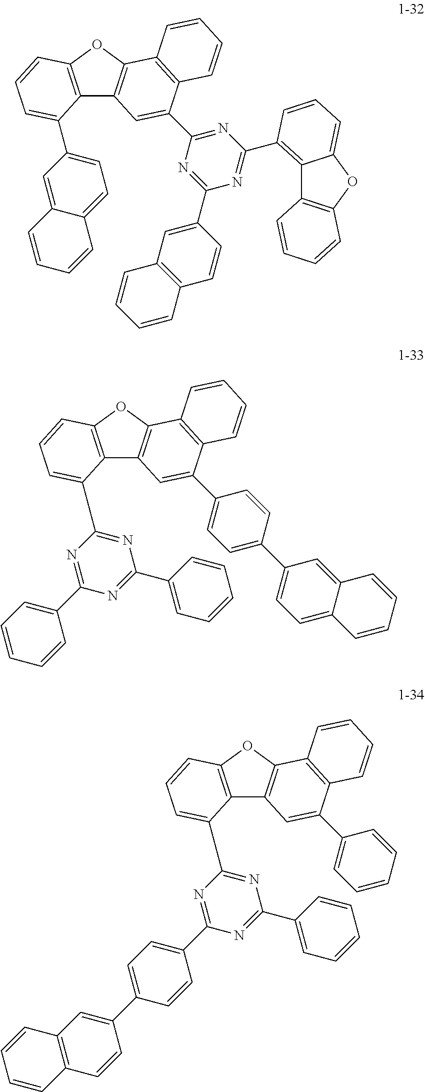 [SMILES: c1ccc2cc(-c3nc(-c4cc5c(oc6cccc(-c7ccc8ccccc8c7)c65)c5ccccc45)nc(-c4cccc5oc6ccccc6c45)n3)ccc2c1 ; c1ccc(-c2nc(-c3ccccc3)nc(-c3cccc4oc5c6ccccc6c(-c6ccc(-c7ccc8ccccc8c7)cc6)cc5c34)n2)cc1 ; c1ccc(-c2nc(-c3ccc(-c4ccc5ccccc5c4)cc3)nc(-c3cccc4oc5c6ccccc6c(-c6ccccc6)cc5c34)n2)cc1]

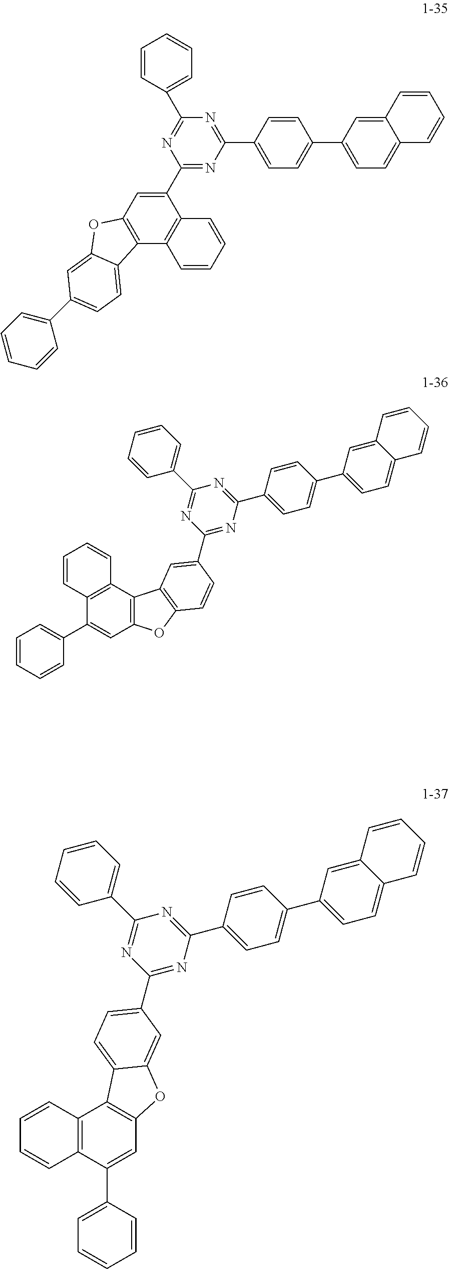 [SMILES: c1ccc(-c2ccc3c(c2)oc2cc(-c4nc(-c5ccccc5)nc(-c5ccc(-c6ccc7ccccc7c6)cc5)n4)c4ccccc4c23)cc1 ; c1ccc(-c2nc(-c3ccc(-c4ccc5ccccc5c4)cc3)nc(-c3ccc4oc5cc(-c6ccccc6)c6ccccc6c5c4c3)n2)cc1 ; c1ccc(-c2nc(-c3ccc(-c4ccc5ccccc5c4)cc3)nc(-c3ccc4c(c3)oc3cc(-c5ccccc5)c5ccccc5c34)n2)cc1]

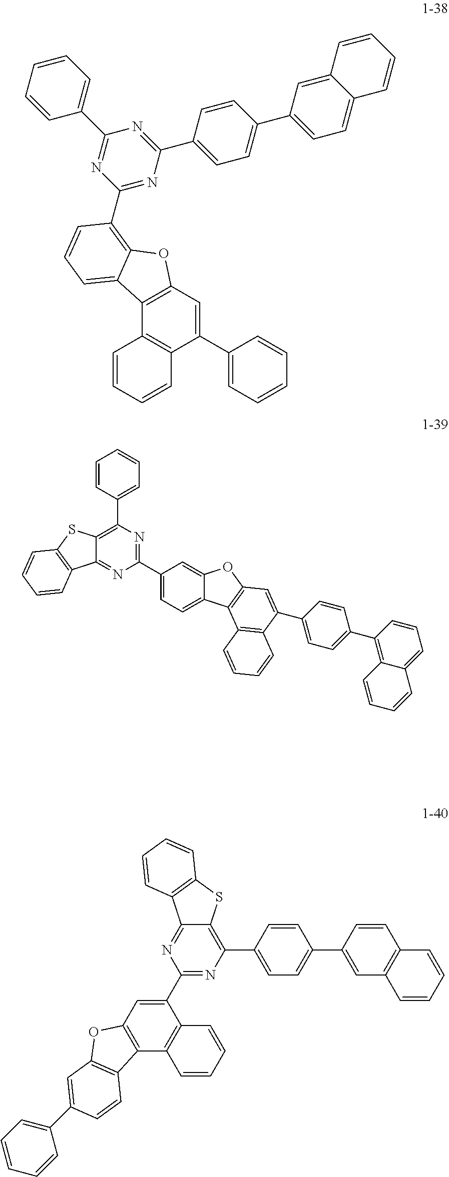 [SMILES: c1ccc(-c2nc(-c3ccc(-c4ccc5ccccc5c4)cc3)nc(-c3cccc4c3oc3cc(-c5ccccc5)c5ccccc5c34)n2)cc1 ; c1ccc(-c2nc(-c3ccc4c(c3)oc3cc(-c5ccc(-c6cccc7ccccc67)cc5)c5ccccc5c34)nc3c2sc2ccccc23)cc1 ; c1ccc(-c2ccc3c(c2)oc2cc(-c4nc(-c5ccc(-c6ccc7ccccc7c6)cc5)c5sc6ccccc6c5n4)c4ccccc4c23)cc1]

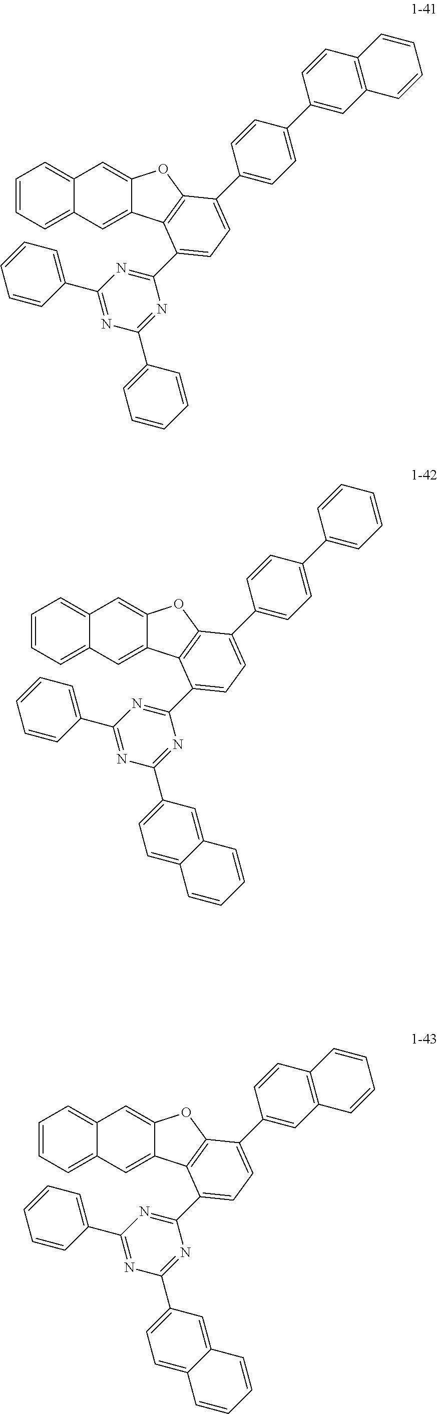 [SMILES: c1ccc(-c2nc(-c3ccccc3)nc(-c3ccc(-c4ccc(-c5ccc6ccccc6c5)cc4)c4oc5cc6ccccc6cc5c34)n2)cc1 ; c1ccc(-c2ccc(-c3ccc(-c4nc(-c5ccccc5)nc(-c5ccc6ccccc6c5)n4)c4c3oc3cc5ccccc5cc34)cc2)cc1 ; c1ccc(-c2nc(-c3ccc4ccccc4c3)nc(-c3ccc(-c4ccc5ccccc5c4)c4oc5cc6ccccc6cc5c34)n2)cc1]

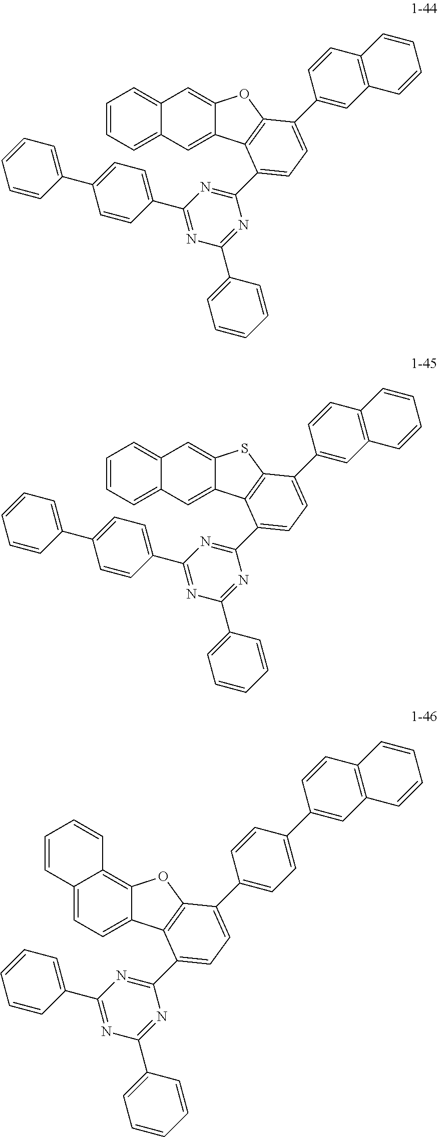 [SMILES: c1ccc(-c2ccc(-c3nc(-c4ccccc4)nc(-c4ccc(-c5ccc6ccccc6c5)c5oc6cc7ccccc7cc6c45)n3)cc2)cc1 ; c1ccc(-c2ccc(-c3nc(-c4ccccc4)nc(-c4ccc(-c5ccc6ccccc6c5)c5sc6cc7ccccc7cc6c45)n3)cc2)cc1 ; c1ccc(-c2nc(-c3ccccc3)nc(-c3ccc(-c4ccc(-c5ccc6ccccc6c5)cc4)c4oc5c6ccccc6ccc5c34)n2)cc1]

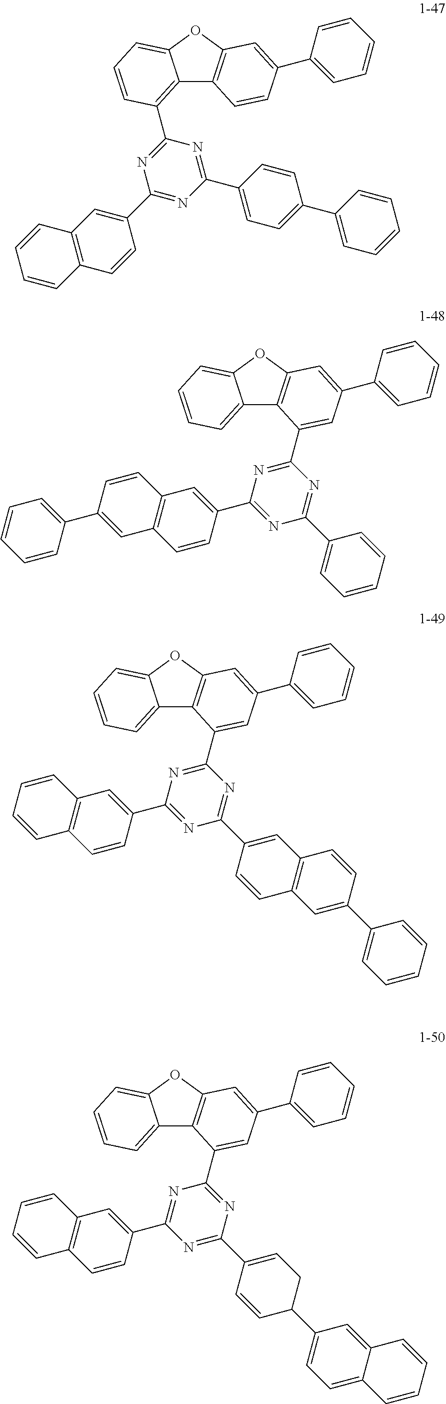 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc3cc(-c4nc(-c5ccccc5)nc(-c5cc(-c6ccccc6)cc6oc7ccccc7c56)n4)ccc3c2)cc1 ; c1ccc(-c2ccc3cc(-c4nc(-c5ccc6ccccc6c5)nc(-c5cc(-c6ccccc6)cc6oc7ccccc7c56)n4)ccc3c2)cc1 ; C1=CC(c2ccc3ccccc3c2)CC=C1c1nc(-c2ccc3ccccc3c2)nc(-c2cc(-c3ccccc3)cc3oc4ccccc4c23)n1]

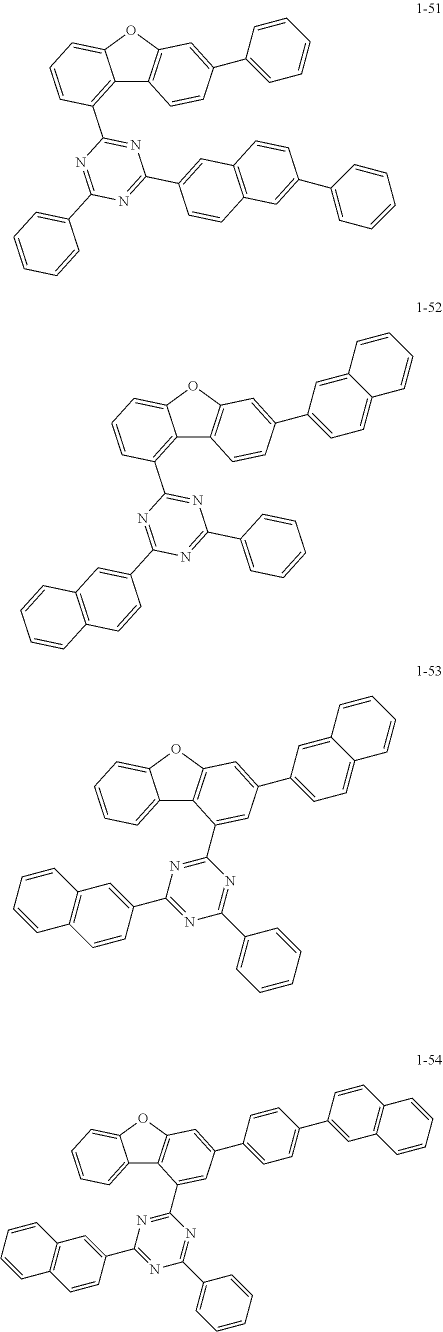 [SMILES: c1ccc(-c2ccc3cc(-c4nc(-c5ccccc5)nc(-c5cccc6oc7cc(-c8ccccc8)ccc7c56)n4)ccc3c2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3ccc4ccccc4c3)nc(-c3cc(-c4ccc(-c5ccc6ccccc6c5)cc4)cc4oc5ccccc5c34)n2)cc1]

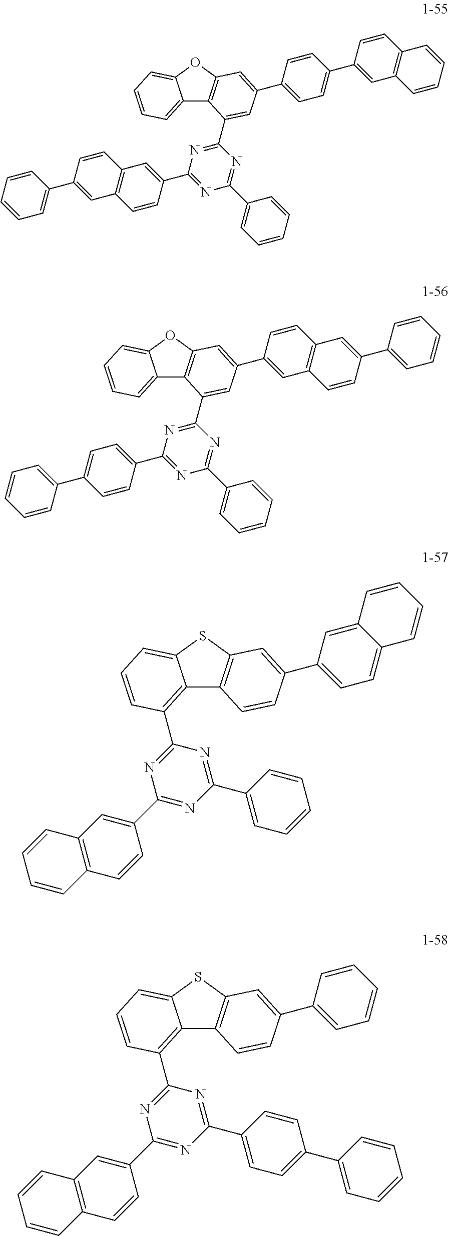 [SMILES: c1ccc(-c2ccc3cc(-c4nc(-c5ccccc5)nc(-c5cc(-c6ccc(-c7ccc8ccccc8c7)cc6)cc6oc7ccccc7c56)n4)ccc3c2)cc1 ; c1ccc(-c2ccc(-c3nc(-c4ccccc4)nc(-c4cc(-c5ccc6cc(-c7ccccc7)ccc6c5)cc5oc6ccccc6c45)n3)cc2)cc1 ; c1ccc(-c2nc(-c3ccc4ccccc4c3)nc(-c3cccc4sc5cc(-c6ccc7ccccc7c6)ccc5c34)n2)cc1 ; c1ccc(-c2ccc(-c3nc(-c4ccc5ccccc5c4)nc(-c4cccc5sc6cc(-c7ccccc7)ccc6c45)n3)cc2)cc1]

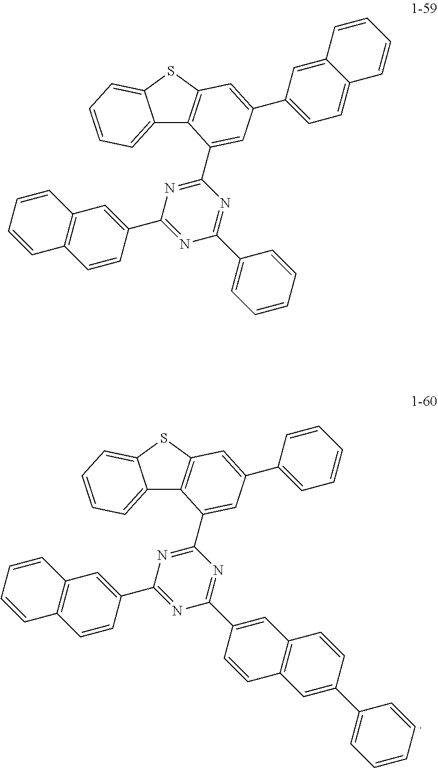 [SMILES: c1ccc(-c2nc(-c3ccc4ccccc4c3)nc(-c3cc(-c4ccc5ccccc5c4)cc4sc5ccccc5c34)n2)cc1 ; c1ccc(-c2ccc3cc(-c4nc(-c5ccc6ccccc6c5)nc(-c5cc(-c6ccccc6)cc6sc7ccccc7c56)n4)ccc3c2)cc1]

17. A composition for an organic material layer, the composition comprising:
(a) the heterocyclic compound represented by Chemical Formula 1 of claim 1; and
(b) a heterocyclic compound represented by the following Chemical Formula A: 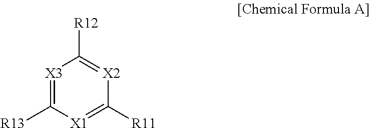
wherein, in Chemical Formula A,
X1 to X3 are the same as or different from each other, and each independently N or CR″;
at least two or more of X1 to X3 are N;
R11 to R13 and R″ are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring; or a substituted or unsubstituted C2 to C60 heteroring; and
at least one of R11 to R13 is a group represented by the following Chemical Formula B-1 or Chemical Formula B-2, 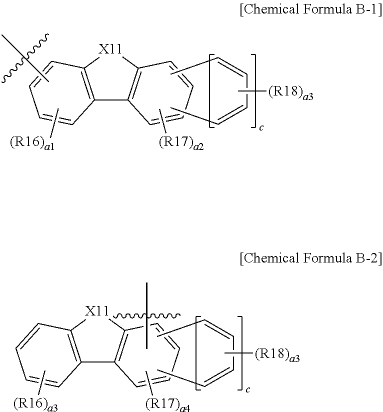
in Chemical Formulae B-1 and B-2,
X11 is O or S;
c is an integer of 0 or 1;
R16 to R18 are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group;
a1 is one of integers of 1 to 3;
a2 is one of integers of 1 to 4, and when c is 1, a2 is 1 or 2;
a3 is one of integers of 1 to 4; and
a4 is one of integers of 1 to 3, and when c is 1, a4 is 1.

18. The composition of claim 17, wherein (a) the heterocyclic compound and (b) the heterocyclic compound have a weight ratio of 1:10 to 10:1.
